(19) United States
(12) Patent Application Publication
(10) Pub. No.: US 2026/0356804 A1
(43) Pub. Date: Nov. 12, 2026

(54) SODIUM CHANNEL BLOCKING COMPOUNDS, DERIVATIVES THEREOF, AND METHODS OF THEIR USE

(71) Applicant: LATIGO BIOTHERAPEUTICS, INC., Thousand Oaks, CA (US)

(72) Inventors: Ashok Bajji, Thousand Oaks, CA (US); Michael D. Kaufman, Thousand Oakes, CA (US); James Michael Balkovec, Martinsville, NJ (US)

(21) Appl. No.: 19/101,138

(22) PCT Filed: Sep. 8, 2023
(86) PCT No.: PCT/US2023/032280

§ 371 Date: Feb. 4, 2025

Related U.S. Application Data
(60) Provisional application No. 63/405,094, filed on Sep. 9, 2022.
(60) Provisional application No. 63/405,096, filed on Sep. 9, 2022.
(60) Provisional application No. 63/405,120, filed on Sep. 9, 2022.
(60) Provisional application No. 63/405,151, filed on Sep. 9, 2022.

(51) Int. Cl. C07D 213/82; A61K 31/444; C07D 213/71; C07D 237/24; C07D 401/04; C07D 401/12; C07D 405/14; C07D 417/14

(52) U.S. Cl. CPC C07D 213/82; A61K 31/444; C07D 213/71; C07D 237/24; C07D 401/04; C07D 401/12; C07D 405/14; C07D 417/14

(57) ABSTRACT

The invention provides compounds that are useful for treatment of conditions associated with aberrant activity of voltage gated sodium channel Nav1.8, and methods of treating a subject with those compounds for conditions such as pain, itch, and cough.

### I. FIELD OF THE INVENTION

The application relates generally to sodium channel blocking compounds, derivatives thereof, and the use of such compounds as pharmacological agents.

### II. BACKGROUND

Millions of people suffer from conditions associated with pain, itch, and/or cough. Pain can be a symptom or cause of conditions such as neuropathy, hyperalgesia, and opioid use disorders. In many cases, drugs used to treat such condition fail to provide relief or produce intolerable side effects. Therefore, existing treatments are inadequate for many patients who suffer from these conditions.

### III. SUMMARY

The invention provides compounds that are useful for treatment of conditions associated with aberrant activity of voltage gated sodium channel Nav1.8, such as pain, itch, and cough.

### (A) First Set of Compounds:

In one aspect, the invention provides a compound of Formula (I):

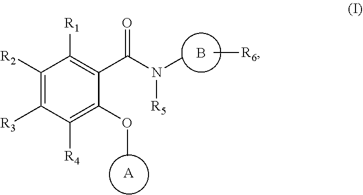

and pharmaceutically acceptable salts, hydrates, and solvates thereof, wherein, A is aryl or heteroaryl wherein the aryl or heteroaryl is unsubstituted or substituted with one or more groups selected from the group consisting of halo-C₁-C₄ alkyl wherein the haloalkyl chain may be fully or partially halogenated, substituted or unsubstituted C₁-C₈ alkyl, deuterated C₁-C₄ alkyl wherein the alkyl chain may be fully or partially deuterated, C₃-C₁₀ cycloalkyl, halogen, cyano, nitro, C₁-C₈ alkoxyl, haloalkoxyl, wherein the haloalkoxy chain may be fully or partially halogenated, or arylalkoxyl;
B is aryl or heteroaryl wherein the aryl may have 1 to 4 substituents and heteroaryl may have 1 to 3 substituents, these substituents are independently selected from halogen, C₁-C₈ alkyl, haloalkyl, or alkoxy;
R₁, R₂, R₃ and R₄ are independently selected from H, halogen, —OH, C₁-C₆-alkyl, C₁-C₆ fluoroalkyl wherein the fluoroalkyl chain may be fully or partially fluorinated, C₃-C₈ branched alkyl, C₃-C₈ branched fluoroalkyl wherein the branched fluoroalkyl chain may be fully or partially fluorinated, alkenyl, alkynyl, haloalkyl, alkoxy, cycloalkoxy, haloalkoxy, thioalkoxy, nitro, cyano, —C(R′)(R″)-cycloalkyl, C(R′)(R′)-aryl, —NR′R″, substituted or unsubstituted 3-8 membered cycloalkyl, 3-8 membered cycloalkenyl, or 3-6 membered heterocycloalkyl, wherein the 3-6 membered heterocycloalkyl comprises at least one heteroatom independently selected from O, S, and N;
R₅ is H or C₁-C₃ alkyl; and
R₆ is —C(═O)NH₂, —C(═O)NHR′, —C(═O)NR′R″, or Formula (II):

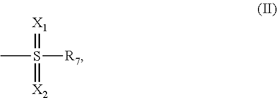

wherein:
X₁ and X₂ are both O, NH, or NR′; or
X₁ is O and X₂ is either NH or NR′;
R₇ is NH₂, NHR′, NR′R″, C₁-C₃ alkyl, C₃-C₈ cycloalkyl, haloalkyl, halocycloalkyl, aryl, heteroaryl, or heterocyclyl.

In various embodiments, the compound of the invention is a compound of Formula (III):

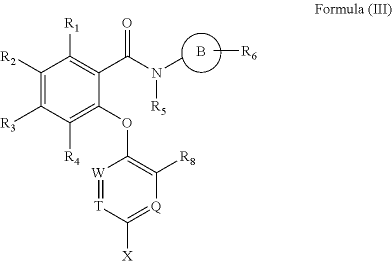

wherein, R₁, R₂, R₃, R₄, R₅, R₆, and B are described in Formula (I);
Q, T and W is independently N or CR₉;
R₉ is H, halogen, —CD₃, alkyl, haloalkyl, alkoxy, haloalkoxy, cyano, —CF₃, —OCF₃, or substituted or unsubstituted cycloalkoxy;
X is H, halogen, —CD₃, alkyl, haloalkyl, alkoxy, cycloalkoxy, haloalkoxy, cyano, —CF₃, — or OCF₃;
R₈ is H, hydroxyl, halogen, —CD₃, C₁-C₆-alkyl, branched alkyl, haloalkyl where the alkyl chain is fully or partially halogenated, alkoxy, arylalkoxy, cycloalkoxy, haloalkoxy, cyano, —CH₂-cycloalkyl, —CH(CH₃)-cycloalkyl, trifluoromethyl, cyclopropylmethyl, or substituted or unsubstituted 3-6 membered cycloalkyl.

In another embodiment, B is

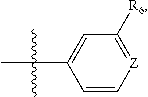

wherein R₆ is described above; Z is CR₁₀, N, or N⁻O⁻; wherein R₁₀ is H, halo, —CD₃, C₁-C₈ alkyl, haloalkyl, or alkoxy.

In another embodiment, R₆ is

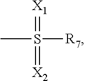

wherein:
X₁ and X₂ are both O, NH, or NR′; or
X₁ is O and X₂ is either NH or NR′;
R₇ is NH₂, NHR′, NR′R″, C₁-C₃ alkyl, C₃-C₈ cycloalkyl, haloalkyl, halocycloalkyl, aryl, heteroaryl, or heterocyclyl.

In another embodiment, R₆ is

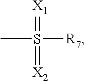

wherein:
X₁ is O, X₂ is NH, and R₇ is C₁-C₃ alkyl.

In another embodiment, R₆ does not include —C(═O)NH₂, —C(═O)NHR′, or —C(═O)NR′R″.

In another embodiment, B is a phenyl ring.

In another embodiment, B is a pyridine ring.

In another embodiment, R₁ is H, —CH₃, or F.

In another embodiment, R₂ is chloro, —CF₃, H, 2-pyrazoline, or 1-methyl-1H-pyrazol-4-yl.

In another embodiment, R₃ is H, —CF₃, or F.

In another embodiment, R₄ is H.

In another embodiment, R₈ is H, —CH₃, or —O—CH₃.

In another embodiment, R₇ is —CH₃.

In another embodiment, X is F or —CN.

In another embodiment, Q is N or CH.

In another embodiment, W is CH and T is CH.

In another embodiment, Z is CR₁₀ and R₁₀ is H or F.

In another embodiment, the compound is selected from a group consisting of:

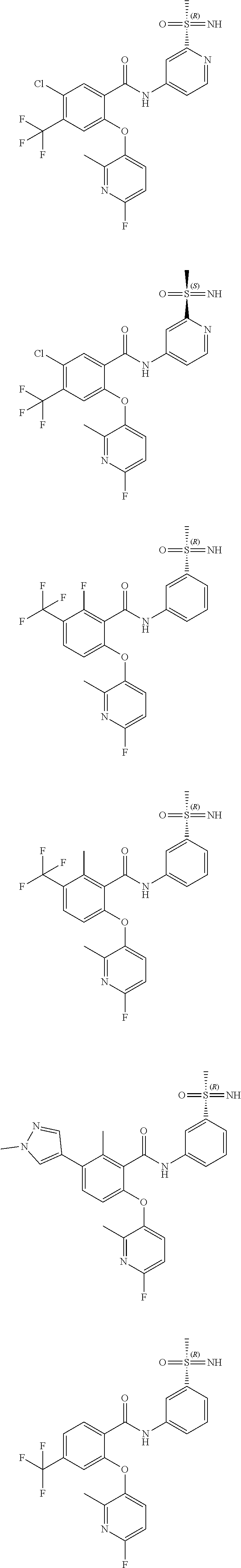

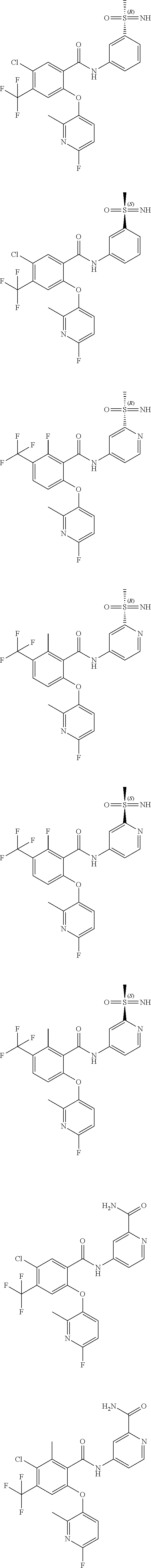

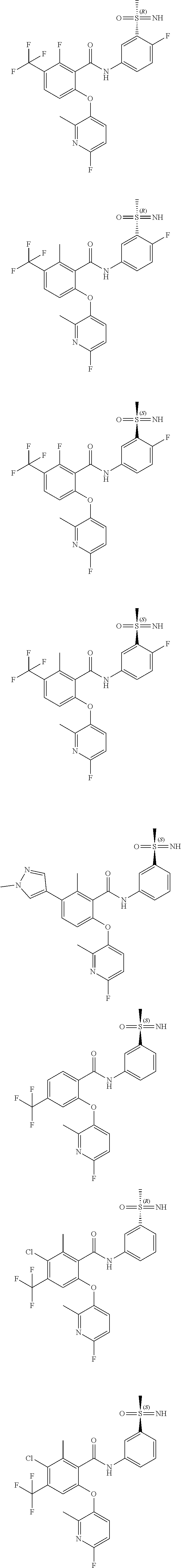

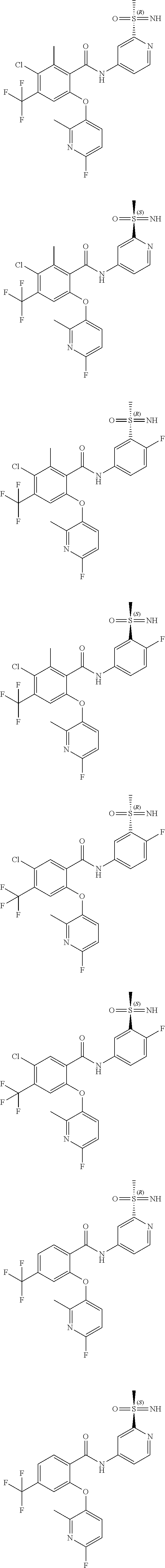

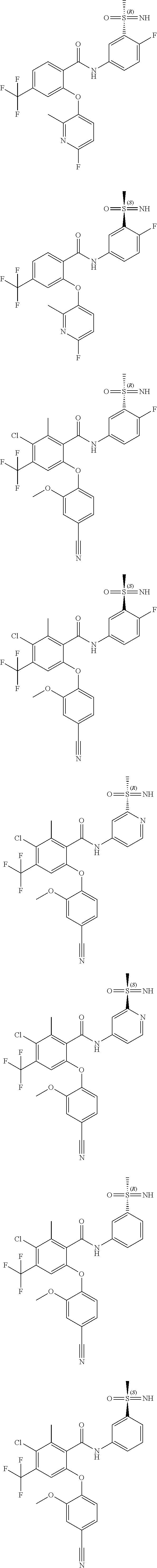

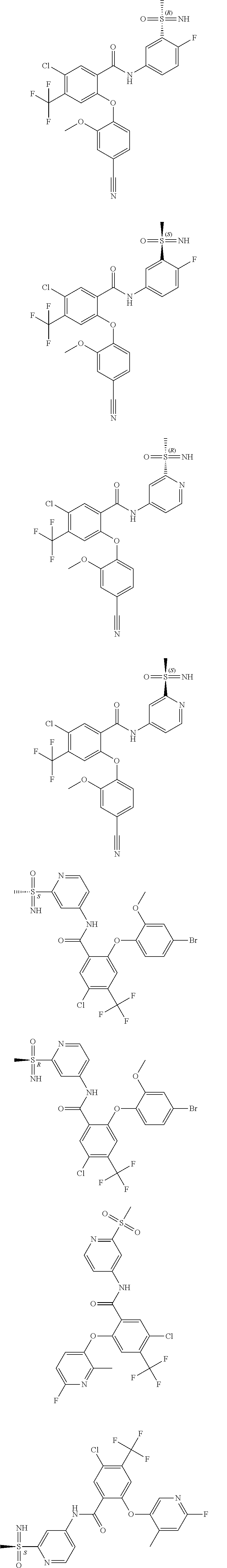

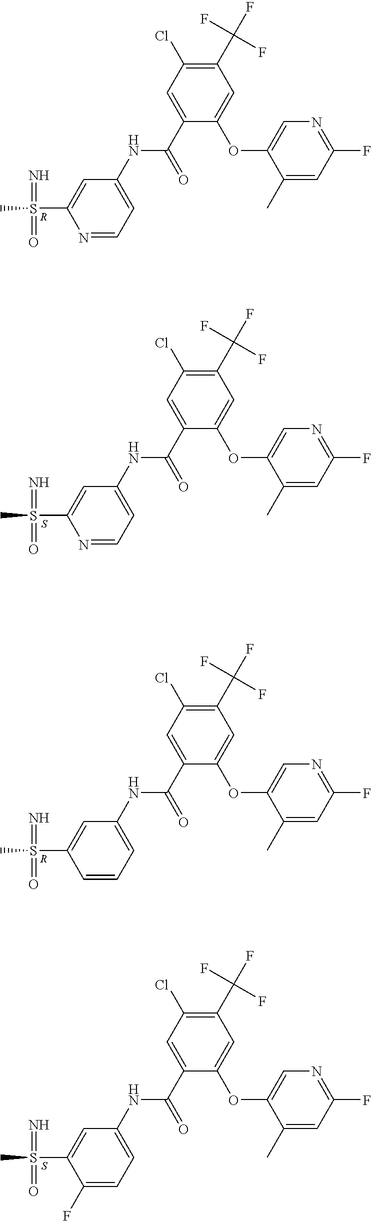

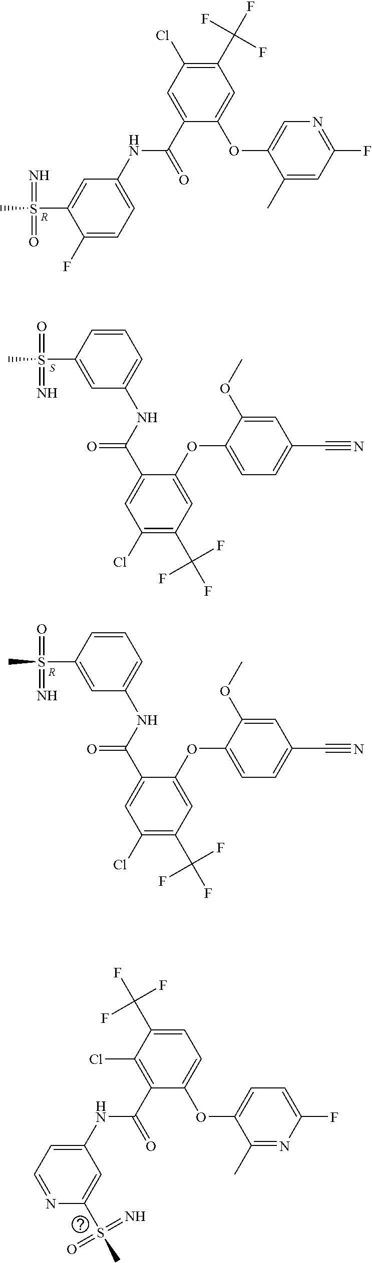

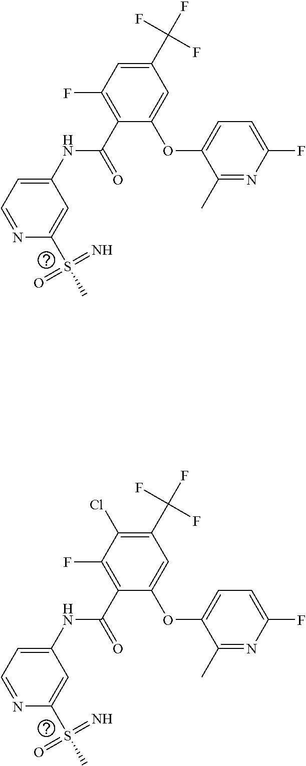

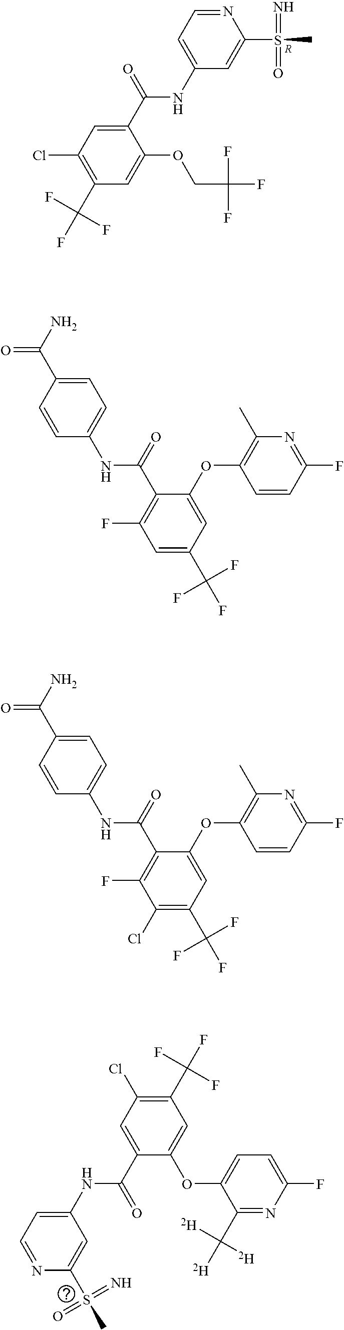

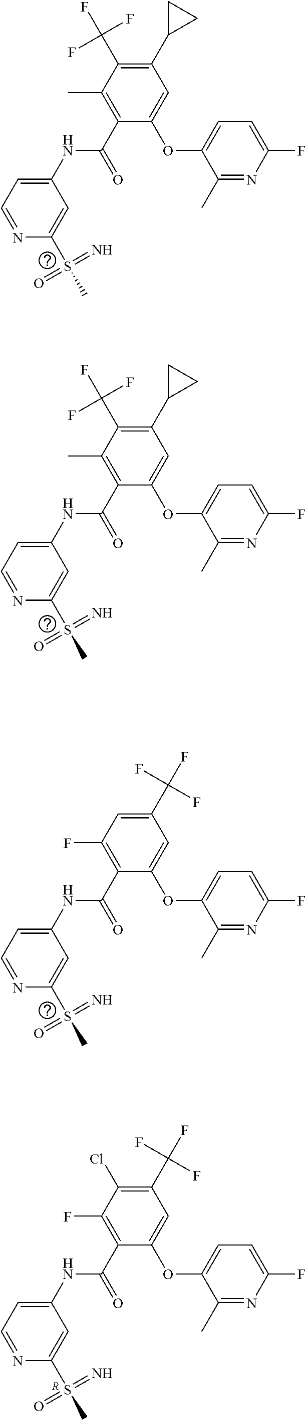

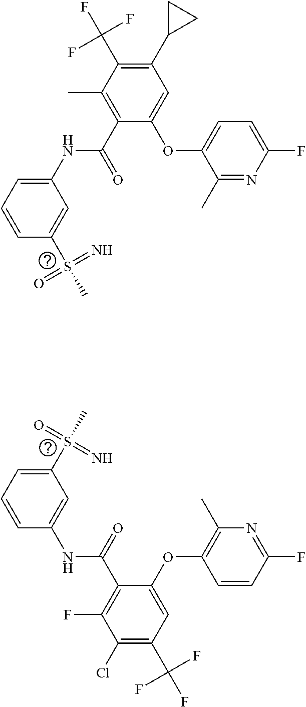

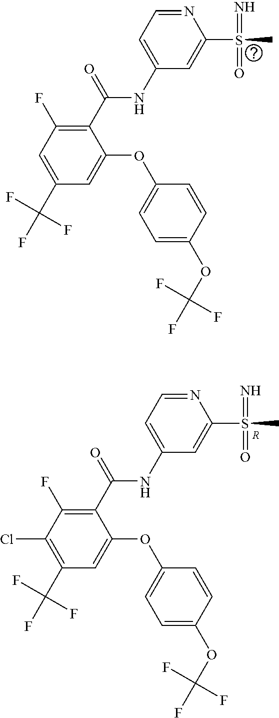

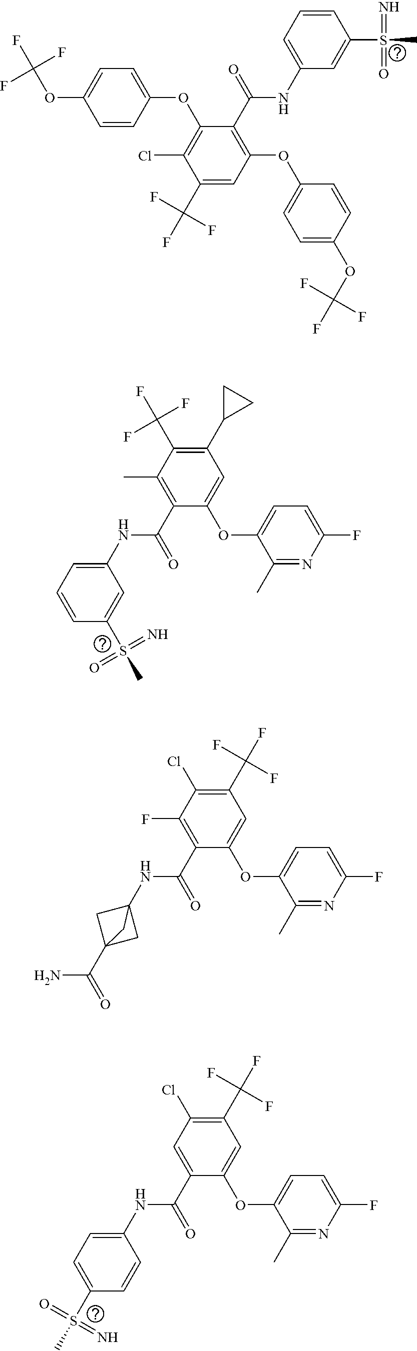

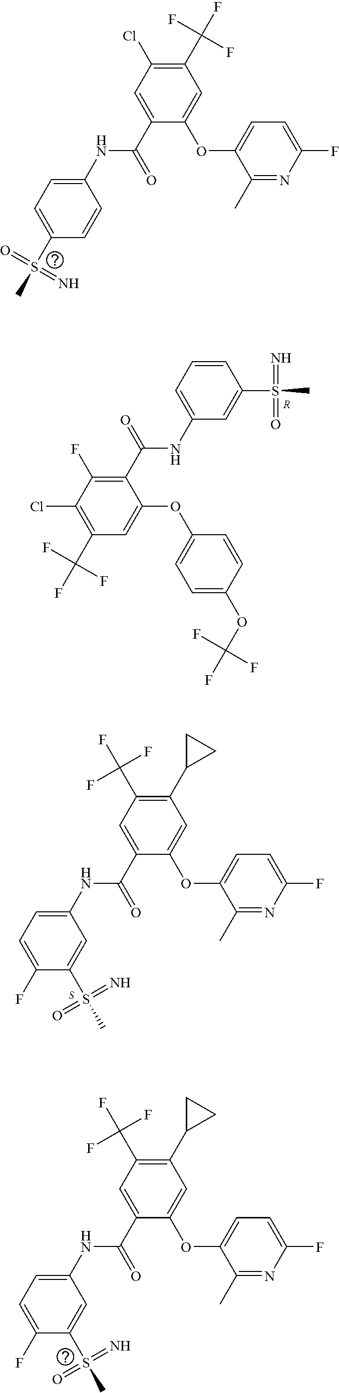

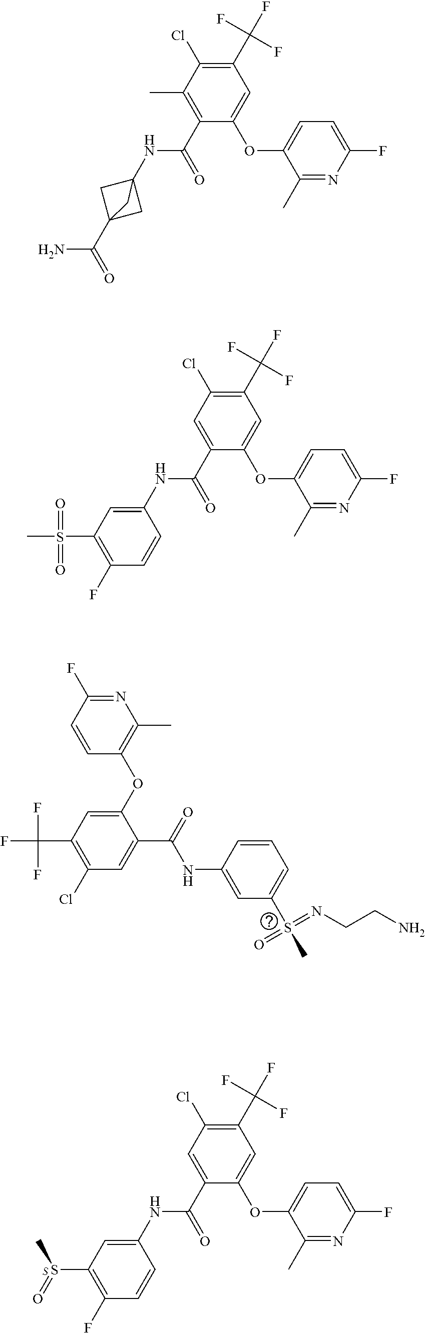

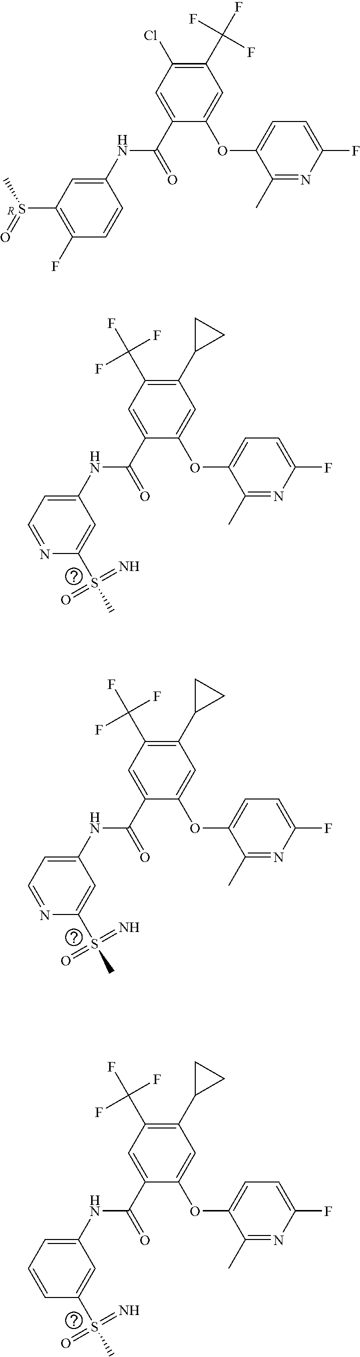

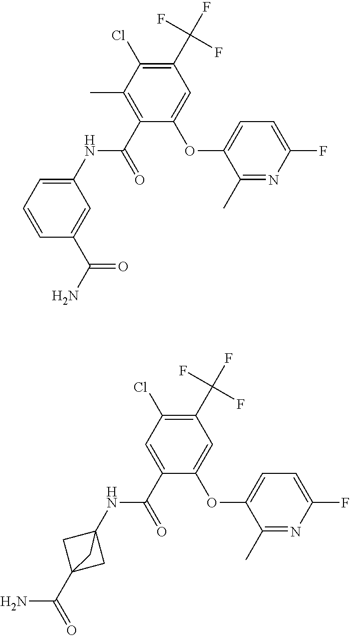

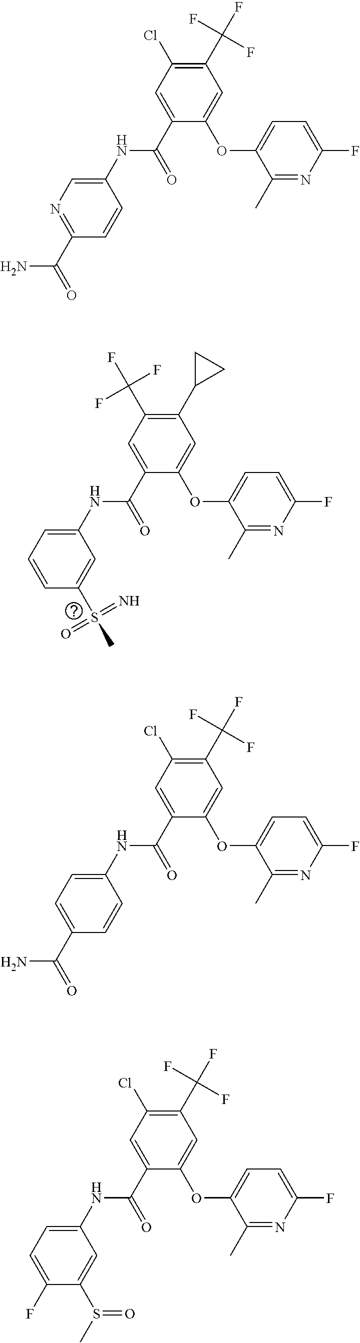

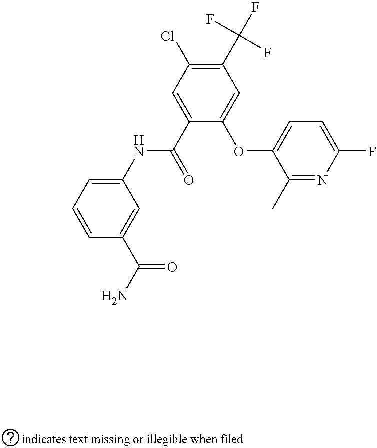

### (B) Second Set of Compounds

In one aspect, the invention provides a compound of Formula (I):

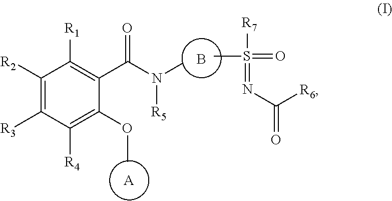

and pharmaceutically acceptable salts, hydrates, and solvates thereof, wherein:
A and B are independently aryl or heteroaryl, wherein the aryl or heteroaryl is unsubstituted or substituted with one or more groups selected from the group consisting of halo-C₁-C₄ alkyl wherein the haloalkyl chain may be fully or partially halogenated, substituted or unsubstituted C₁-C₈ alkyl, deuterated C₁-C₄ alkyl wherein the alkyl chain may be fully or partially deuterated, C₃-C₁₀ cycloalkyl, halogen, cyano, nitro, C₁-C₈ alkoxyl, aloalkoxyl wherein the haloalkyoxy chain may be fully or partially halogenated, and arylalkoxyl;
R₁, R₂, R₃ and R₄ are independently selected from hydrogen, —OH, halogen, C₁-C₆-alkyl, C₁-C₆ fluoroalkyl wherein the fluoroalkyl chain may be fully or partially fluorinated, C₃-C₈ branched alkyl, C₃-C₈ branched fluoroalkyl wherein the branched fluoroalkyl chain may be fully or partially fluorinated, alkenyl, alkynyl, haloalkyl, alkoxy, cycloalkoxy, haloalkoxy, thioalkoxy, nitro, cyano, —C(R′)(R″)-cycloalkyl, C(R′)(R′)-aryl, NR′R″, 3-8, membered cycloalkyl, 3-8-membered cycloalkenyl 3-6 membered heterocycloalkyl, any of which may have one or more substituents, wherein the 3-6 membered heterocycloalkyl comprises at least one heteroatom independently selected from O, S, and N, a saturated or unsaturated 5 or more membered ring or an aryl ring optionally containing 1 or more heteroatoms independently selected from 0, S, and N, wherein each 5 or more membered ring is unsubstituted or substituted with one to five substituents selected from hydrogen, cyano, halo, or methyl; a fused ring formed by at least two of R₁, R₂, R₃ and R₄ wherein the fused ring is selected from a group consisting of: optionally saturated carbocyclyl or heterocyclyl containing 5-6 ring members, wherein the heterocyclyl include one or more heteroatoms;
R₅ is H, or substituted or unsubstituted C₁-C₃ alkyl;
R₆ is —(CH₂)ₙRₐ, —(CR_{b}R_{c})ₙRₐ, —(CR_{b}R_{c})ₙ—(OCH₂CH₂)ₙRₐ, —(CR_{b}R_{c})ₙ—(NR′CH₂CH₂)ₙRₐ, —(CR_{b}R_{c})ₙ—(NR′CH₂CH₂O)ₙRₐ, or —(CR_{b}R_{c})ₙ—(NHCH₂CH₂NH)ₙRₐ;
   wherein Rₐ is H, C₁-C₆-alkyl, C₂-C₈ branched alkyl, C₃-C₈ cycloalkyl, aryl, heteroaryl, 4-7 member heterocyclyl, alkenyl, alkynyl, haloalkyl, OH, alkoxy, cycloalkoxy, haloalkoxy, —(OCH₂CH₂)ₙR_{b}, —NR′R″, —COONR′R″, —COOR′, alkylsulfonyl, arylsulfonyl, or —SO₂NR′R″;
   R_{b} is independently selected from H, F, C₁-C₆-alkyl, haloalkyl, branched alkyl, aryl, heteroaryl, 3-7 member carbocyclyl, 4-7 member heterocyclyl with one or more heteroatoms;
   R_{c} is H, C₁-C₆ alkyl or F;
   R_{c} and R_{b} optionally form a 3-6 member carbocyclic ring or 4-6 member heterocyclic ring with one or more hetero atoms;
   R₇ is NH₂, —NHR′, C₁-C₃ alkyl, substituted or unsubstituted C₃-C₄ cycloalkyl, haloalkyl, halocycloalkyl, aryl, heteroaryl, or heterocyclyl.

In various embodiments, the compound of the invention is a compound of Formula (II):

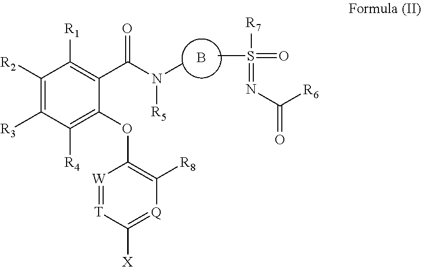

wherein ring B, and groups R₁, R₂, R₃, R₄, R₅, R₆, and R₇, are described in claim 1;
R₈ is H, hydroxyl, halogen, —CD₃, C₁-C₆-alkyl, branched alkyl, haloalkyl where the alkyl chain is fully or partially halogenated, alkoxy, arylalkoxy, cycloalkoxy, haloalkoxy, cyano, —CH₂-cycloalkyl, —CH(CH₃)-cycloalkyl, trifluoromethyl, cyclopropylmethyl, 3-6 membered cycloalkyl, or 3-6 membered heterocycloalkyl, any of which may have one or more substituents, wherein the 3-6 membered heterocycloalkyl comprises at least one heteroatom independently selected from O, S, and N;
Q, T, and W are independently selected from N or CR₉;
R₉ is H, halogen, —CD₃, alkyl, haloalkyl, alkoxy, haloalkoxy, cyano, —CF₃, —OCF₃, or cycloalkoxy, each of which is optionally substituted;
X is H, halogen, —CD₃, alkyl, haloalkyl, alkoxy, cycloalkoxy, haloalkoxy, cyano, —CF₃, or —OCF₃.

In another embodiment, the compound of the invention is a compound of Formula (III):

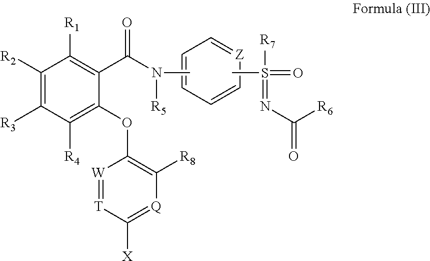

wherein, R₁, R₂, R₃, R₄, R₅, R₆, and R₇ are described above in Formula (I);
R₈, W, T, Q, and X are described above in Formula (II);
Z is CH, N, CF, or N⁺—O⁻

In another embodiment, X is F.

In another embodiment, R₈ is —CH₃.

In another embodiment, Q is N.

In another embodiment, Q is N, W is CH, and T is CH.

In another embodiment, R₁ is H or F.

In another embodiment, R₂ is H, chloro, or CF₃.

In another embodiment, R₃ is H or CF₃.

In another embodiment, R₄ is H.

In another embodiment, R₅ is H.

In another embodiment, R₇ is methyl.

In another embodiment, R₆ is azetidine, pyrrolidine, —CH₂—OH, —CH—(CH₃)—OH, —CH—CH₂—NH—CH₃, —CH₂—NH₂, CH—(CH₃)—NH₂, —CH₂—NH₂, —C—(CH₃)₂—NH₂, or -cyclobutyl-NH₂.

In another embodiment, R₉ is H.

In another embodiment, the compound of Formula (I) is selected from the group consisting of:

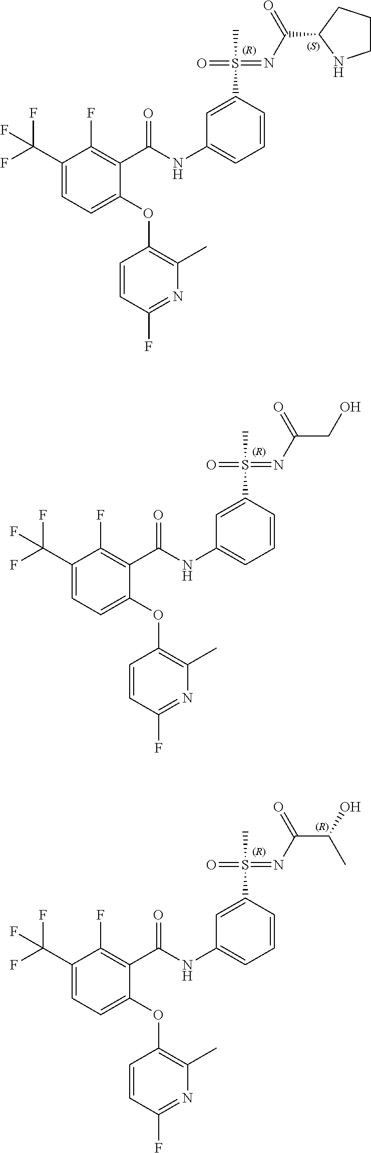

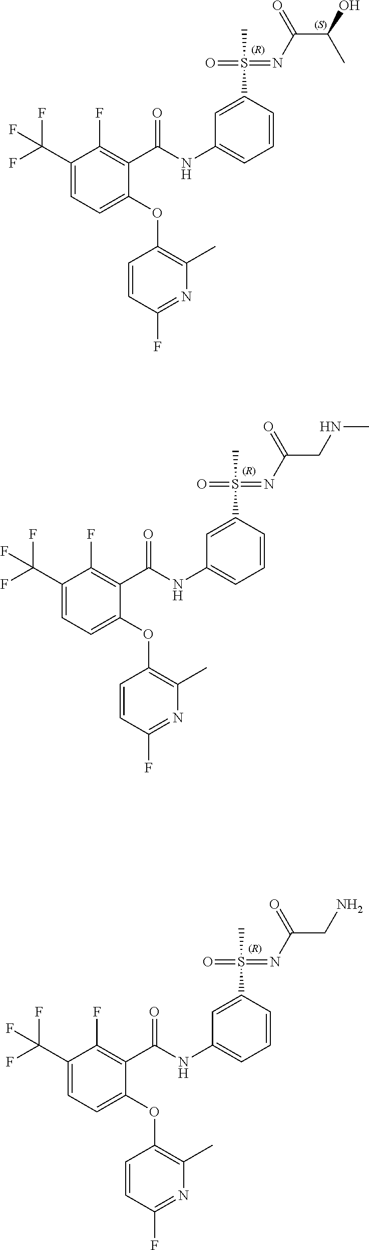

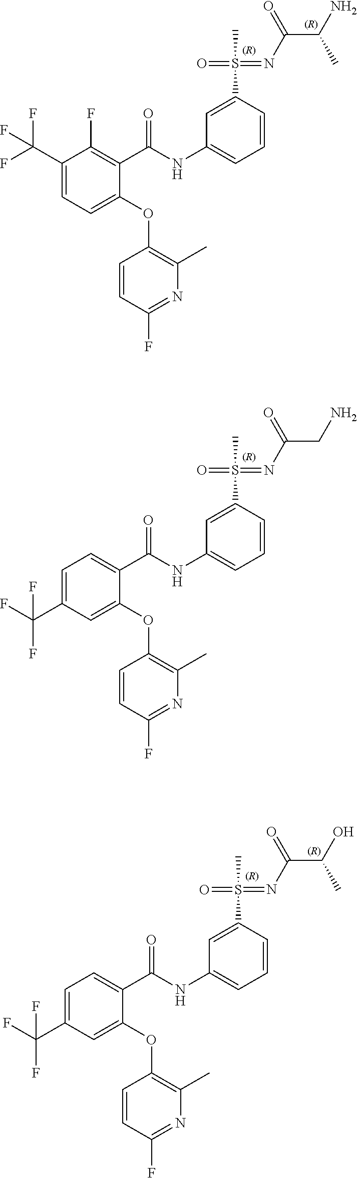

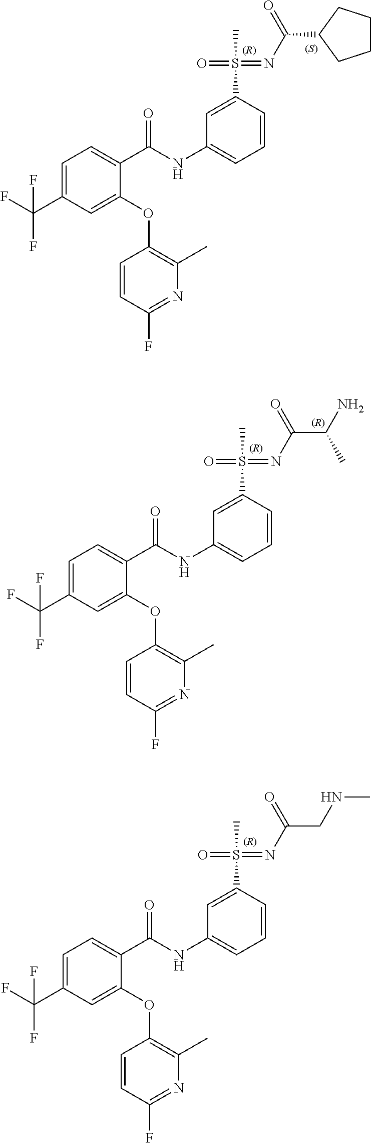

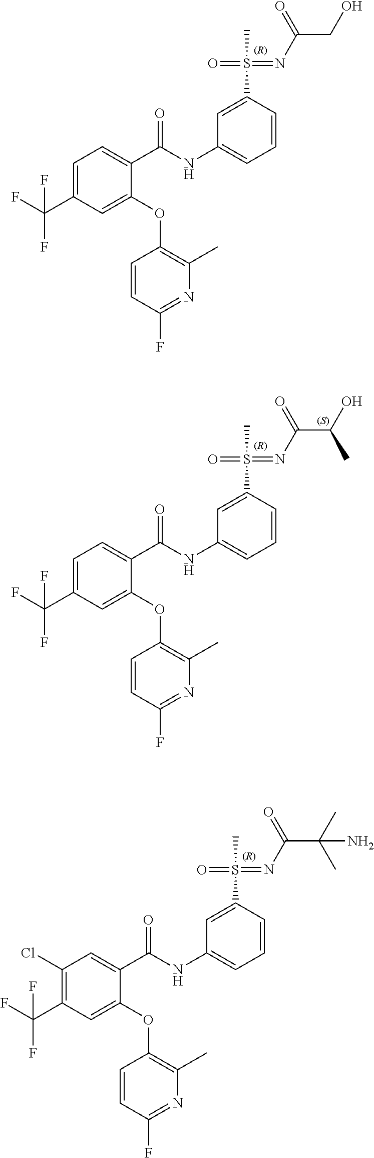

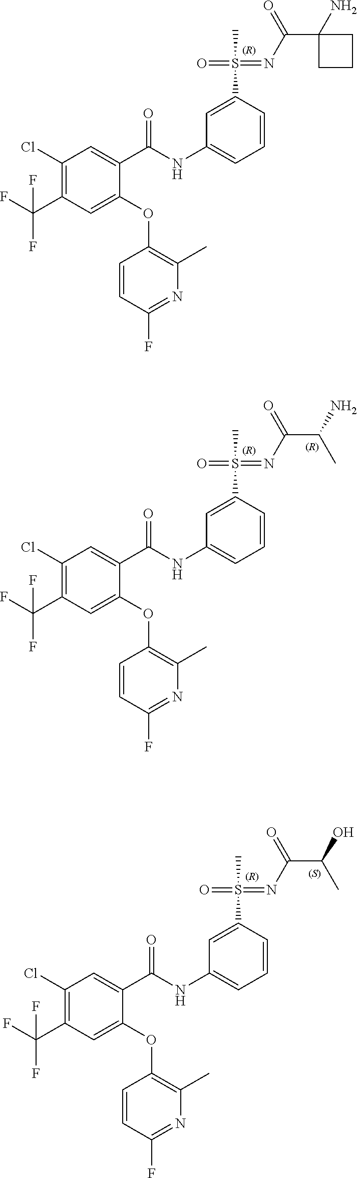

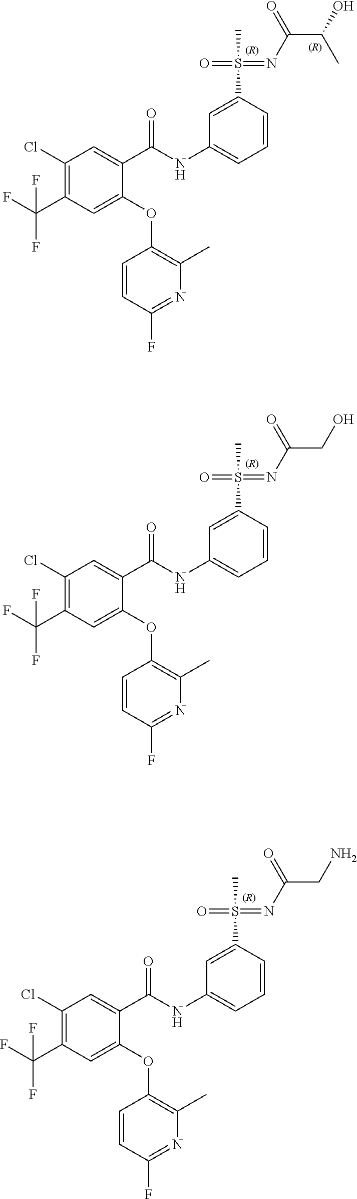

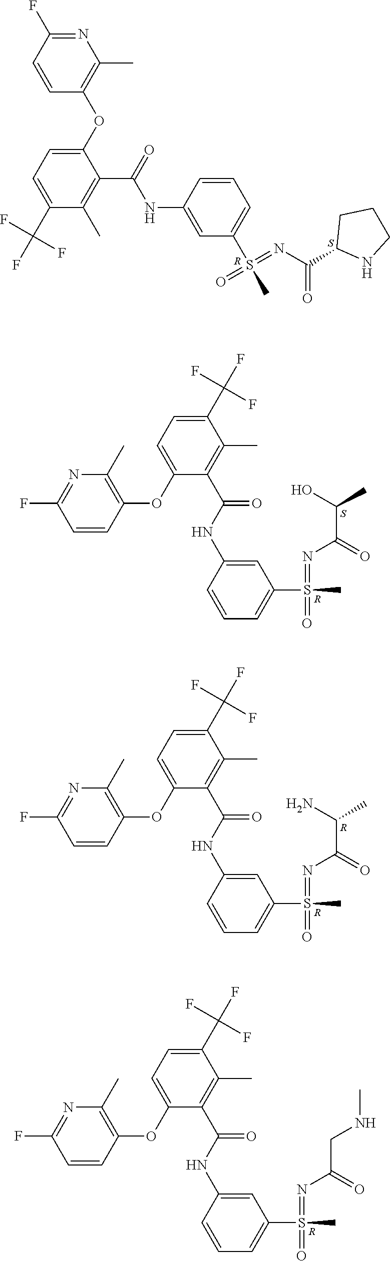

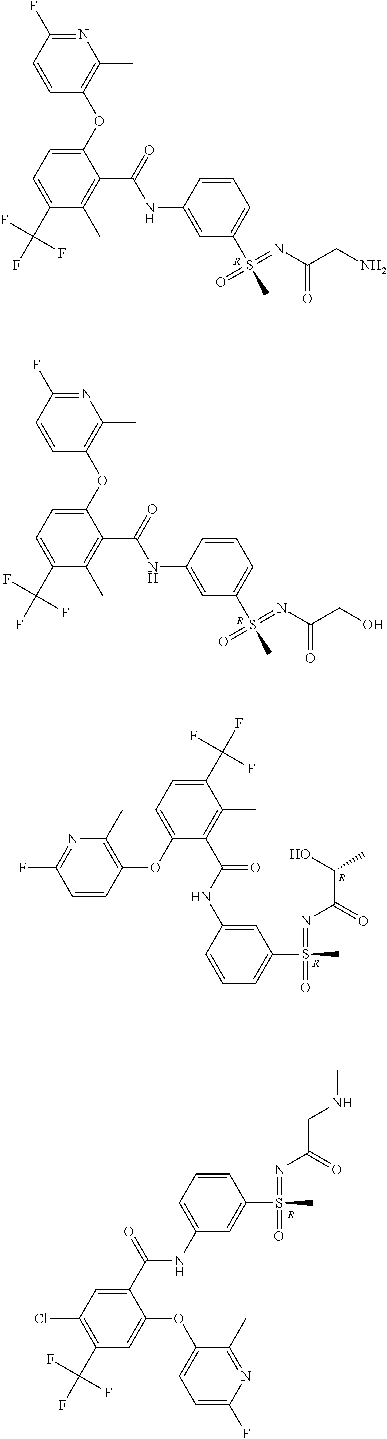

### (C) Third Set of Compounds

In one aspect, the invention provides a compound of compound of Formula (I):

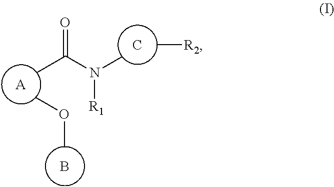

and pharmaceutically acceptable salts, hydrates and solvates thereof, wherein:
A is a substituted or unsubstituted heteroaryl ring comprising at least one heteroatom selected from a group consisting of O, S, or N;
wherein the one or more substitutions on A are selected from H, —OH, halo, C₁-C₈-alkyl, C₁-C₈ fully or partially fluorinated fluoroalkyl, C₂-C₈ branched alkyl, alkenyl, alkynyl, haloalkyl, alkoxy, cycloalkoxy, haloalkoxy, nitro, cyano, —C(R′)(R″)-cycloalkyl, C(R′)(R″)-aryl, —NR′R″, substituted or unsubstituted 3-8 membered cycloalkyl, 3-8 membered cycloalkenyl, or 3-6 membered heterocycloalkyl, wherein the 3-6 membered heterocycloalkyl comprises at least one heteroatom independently selected from O, S, and N;
B is substituted or unsubstituted aryl or heteroaryl, wherein the substitutions are selected from the group consisting of substituted or unsubstituted C₁-C₈ alkyl, deuterated C₁-C₄ alkyl wherein the alkyl chain may be fully or partially deuterated, halo-C₁-C₄ alkyl where the alkyl chain is fully or partially halogenated, C₃-C₁₀ cycloalkyl, halogen, cyano, nitro, C₁-C₈ alkoxyl, haloalkoxyl wherein the haloalkyoxyl chain may be fully or partially halogenated, or arylalkoxyl;
C is substituted or unsubstituted aryl or heteroaryl, wherein the one or more substitutions are independently selected from a group consisting of halo, C₁-C₈ alkyl, haloalkyl, alkoxy;
R₁ is selected from a group consisting of H and C₁-C₄ alkyl;
R₂ is selected from a group consisting of Formula (II):

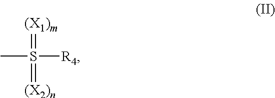

C(═O)NH₂, and —C(═O)NR′R″; wherein,
m and n are independently 0 or 1; and
X₁ is O and X₂ is either NH or NR′; or
X₁ and X₂ are both O, NH, or NR′; or
X₁ is O and X₂ is NR₃; wherein R₃ is selected from a group consisting of: CD₃, C₁-C₄ alkyl or cycloalkyl, haloalkyl, halocycloalkyl, aryl, heteroaryl, and heterocyclyl, (C₁-C₆)alkyl-OH, (C₁-C₆)alkyl-NHR′, (C₁-C₆)alkyl-NR′R″, (C₁-C₆)alkyl-O—(C₁-C₆)alkyl, (C₁-C₆)alkyl-N—(C₁-C₆)alkyl;
R₄ is selected from a group consisting of: —NH₂, —NR′R″, C₁-C₄ alkyl, C₃-C₈-cycloalkyl, haloalkyl, halocycloalkyl, aryl, heteroaryl, and heterocyclyl.

In another embodiment, B is a 6 membered substituted or unsubstituted heteroaryl ring, comprising one or more N atoms. The N atoms in the heteroaryl ring may be in the form of a N-oxide. In certain embodiments, the N-oxide containing B-ring is selected from pyridyl N-oxide, pyrazinyl N-oxide, and pyrimidinyl N-oxide.

In another embodiment, the compound is a compound of Formula (III):

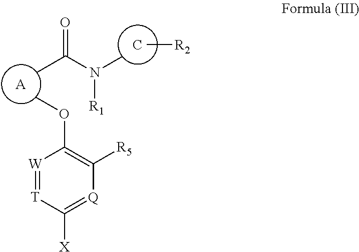

wherein, Q, T and W are independently N or CR₆;
R₆ is H, halogen, —CD₃, alkyl, cycloalkyl, haloalkyl, alkoxy, haloalkoxy, cyano, —CF₃, —OCF₃, or substituted or unsubstituted cycloalkoxy;
R₅ is H, —OH, halo, —CD₃, C₁-C₆-alkyl, branched alkyl, haloalkyl where the alkyl chain is fully or partially halogenated, alkoxy, arylalkoxy, cycloalkoxy, haloalkoxy, cyano, —CH₂-cycloalkyl, —CH(CH₃)-cycloalkyl, trifluoromethyl, cyclopropylmethyl, substituted or unsubstituted 3-6 membered cycloalkyl, any of which may have one or more substituents;
X is H, halo, —CD₃, alkyl, haloalkyl, alkoxy, cycloalkoxy, haloalkoxy, cyano, —CF₃, —OCF₃.

In another embodiment, the compound is a compound of Formula (IV):

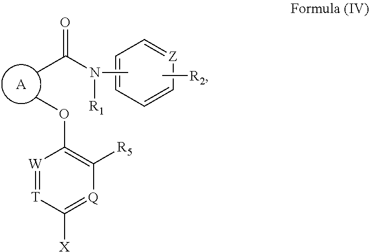

wherein:
Z is CR₁₀, N, or N⁺O⁻; wherein R₁₀ is H, halo, —CD₃, C₁-C₈ alkyl, haloalkyl, or alkoxy.

In certain embodiments, ring A is substituted or unsubstituted 6 or more membered heteroaryl ring having at least one heteroatom independently selected from N, O, or S.

In certain embodiments, ring A is substituted or unsubstituted pyridyl, pyrimidinyl, pyrazinyl, or pyridazinyl.

In certain embodiments, ring A is substituted or unsubstituted 6 membered heteroaryl having at least one heteroatom, wherein the heteroatom is N. In certain embodiments, ring A is substituted or unsubstituted 6 membered heteroaryl having at least 2 N atoms.

In other embodiments, in the compounds of Formula (I), Formula (III), and/or Formula (IV), A is:

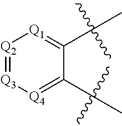

wherein Q₁, Q₂, Q₃ and Q₄ are independently selected from a group consisting of: N, N⁺O⁻, or CR₇; wherein at least two of Q₁, Q₂, Q₃ and Q₄ are CR₇;
R₇ is H, —OH, halo, —CD₃, alkyl, haloalkyl, alkoxy, haloalkoxy, cyano, —CF₃, —OCF₃, substituted or unsubstituted 5 or 6 membered ring heterocyclyl or heteroaryl, saturated heterocyclyl, or partially unsaturated heterocyclyl, O-aryl, O-heteroaryl, O-cycloalkyl, or O-cycloheteroalkyl.

In other embodiments, in the compounds of Formula (I), Formula (III), and/or Formula (IV), A is:

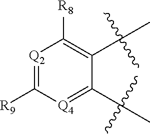

wherein,
Q₂ and Q₄ are independently N or N⁺O⁻;
Q₂ is N or N⁺O⁻; Q₄ is CR₇; or
Q₂ is CR₇, Q₄ is N or N⁺O⁻;
R₈ and R₉ are independently selected from a group consisting of H, —OH, halo, —CD₃, substituted or unsubstituted C₁-C₆ alkyl, branched alkyl, alkenyl, alkylnyl, haloalkyl, alkoxy, cycloalkyl, heterocyclyl, NH₂, NHR′, NR′R″, aryl, heteroaryl, —CF₂CH₃, and —CF₂CF₃; In other embodiments, in the compounds of Formula (I), Formula (III), and/or Formula (IV), A is:

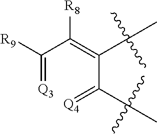

wherein,
Q₃ and Q₄ are independently N or N⁺O⁻;
Q₃ is N or N⁺O⁻ and Q₄ is CR₇; or
Q₃ is CR₇, Q₄ is N or N⁺O⁻; wherein R₇, R₈ and R₉ are defined above.

In other embodiments, in the compounds of Formula (I), Formula (III), and/or Formula (IV), A is:

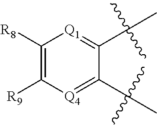

wherein,
Q₁ and Q₄ are independently N or N⁺O⁻;
Q₁ is N or N⁺O⁻, and Q₄ is CR₇;
Q₁ is CR₇ and Q₄ is N or N⁺O⁻; or
Q₁ or Q₄ is CR₇; wherein R₇, R₈, and R₉ are defined above.

In other embodiments, in the compounds of Formula (I), Formula (III), and/or Formula (IV), A is:

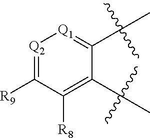

wherein Q₁ and Q₂ are N; wherein R₈ and R₉ are defined above.

In other embodiments, in the compounds of Formula (I), Formula (III), and/or Formula (IV), A is:

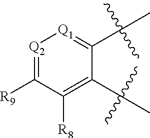

wherein Q₁ is CR₇, Q₂ is N; R₇, R₈, and R₉ are defined above.

In other embodiments, in the compounds of Formula (I), Formula (III), and/or Formula (IV), A is:

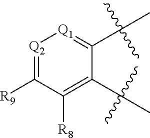

wherein, Q₁ is N; Q₂ is CR₇; R₇, R₈, and R₉ are defined above.

In other embodiments, in the compounds of Formula (I), Formula (III), and/or Formula (IV), R₂ is:

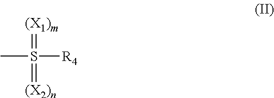

wherein m and n are independently 0 or 1;
X₁ is O and X₂ is NH, and R₄ is alkyl, for example, methyl.

In other embodiments, in the compounds of Formula (I), Formula (III), and/or Formula (IV), R₂ is:

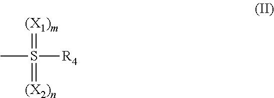

wherein m and n are independently 0 or 1;
X₁ is O and X₂ is O, and R₄ is NH₂ or alkyl, for example, methyl.

In other embodiments, R₂ is —S(═O)CH₃. In another embodiments, in the compound of Formula (III) and/or Formula (IV), Q is N or CH; R₅ is methyl or —OMe, X is F or CN. In other embodiments, in the compound of Formula (III) and/or Formula (IV), Q is CF. In other embodiments, R₇, R₈, and R₉ are independently selected from a group consisting of: H, methyl, fluoro, chloro, bromo, CF₃, cyclopropyl, difluorophenyl, and dimethylpyrazole. In other embodiments, in the compound of Formula (III) and/or Formula (IV), R₁₀ is H or F. In other embodiments, in the compound of Formula (III) and/or Formula (IV), Z is N or CH.

In certain embodiments of the invention, R₂ does not comprise —C(═O)NH₂ or —C(═O)NR′R″.

In certain embodiments, R₂ is Formula (II):

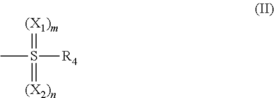

wherein,
m and n are independently 0 or 1; and
X₁ and X₂ are not both O, NH, or NR′.

In other embodiments, the compound of the invention is selected from a group consisting of:

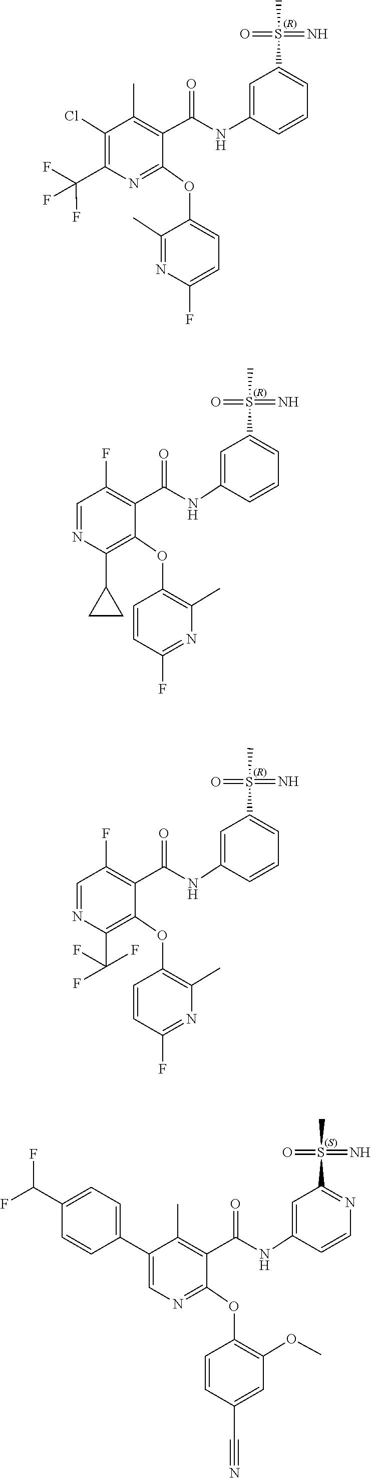

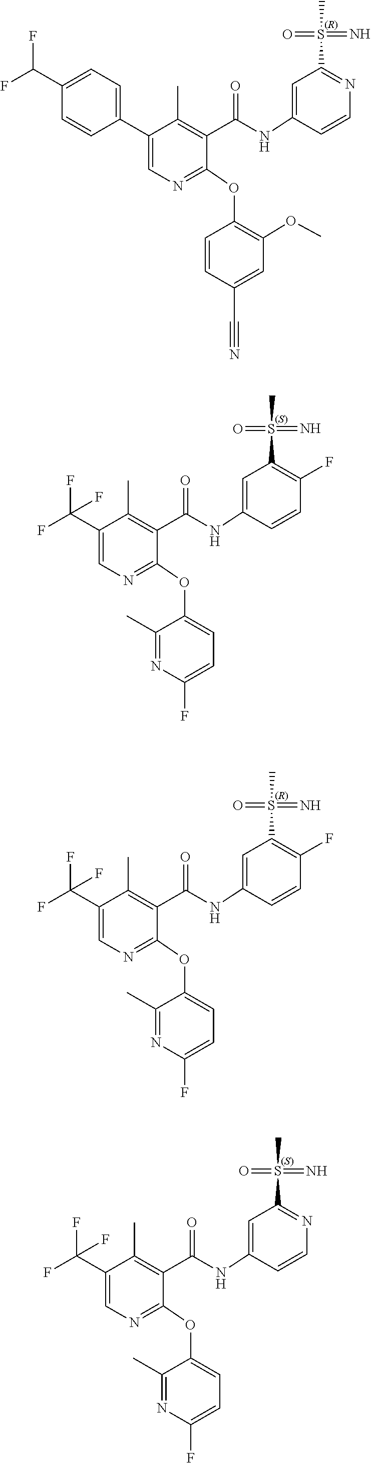

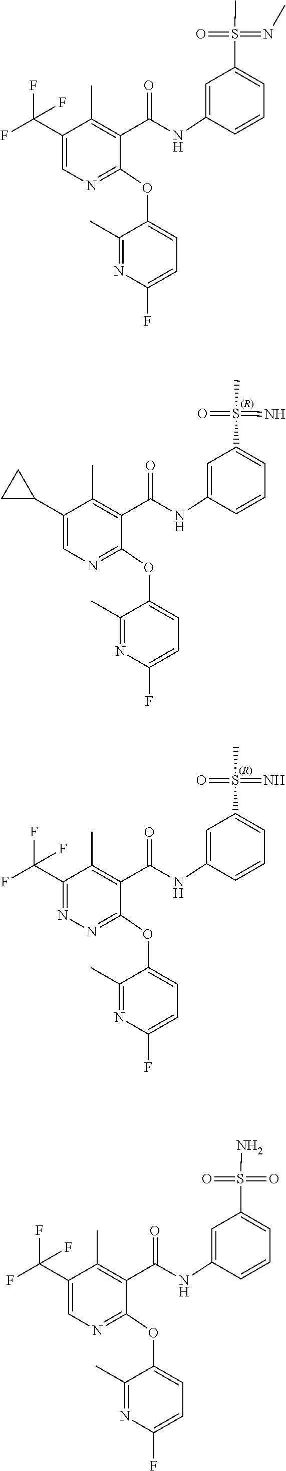

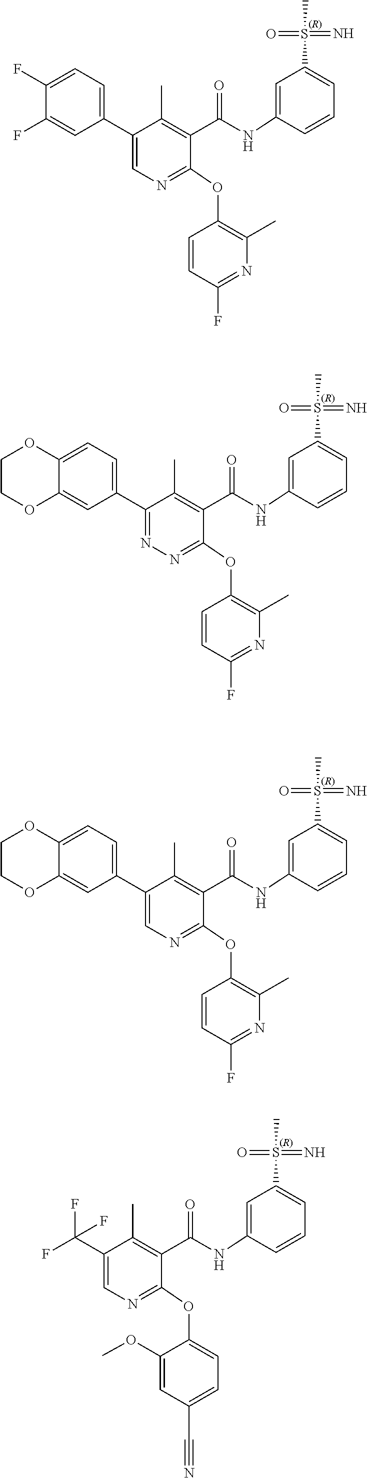

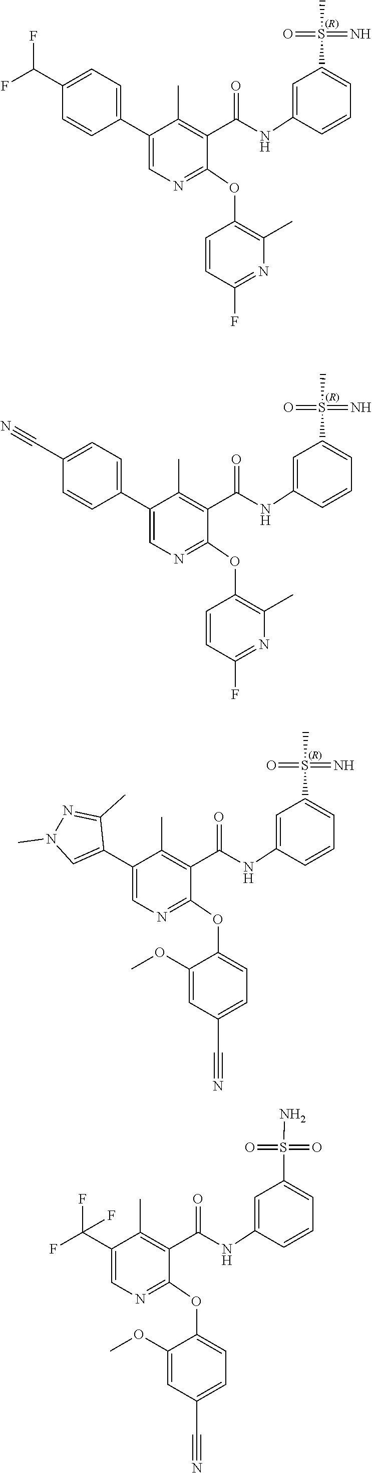

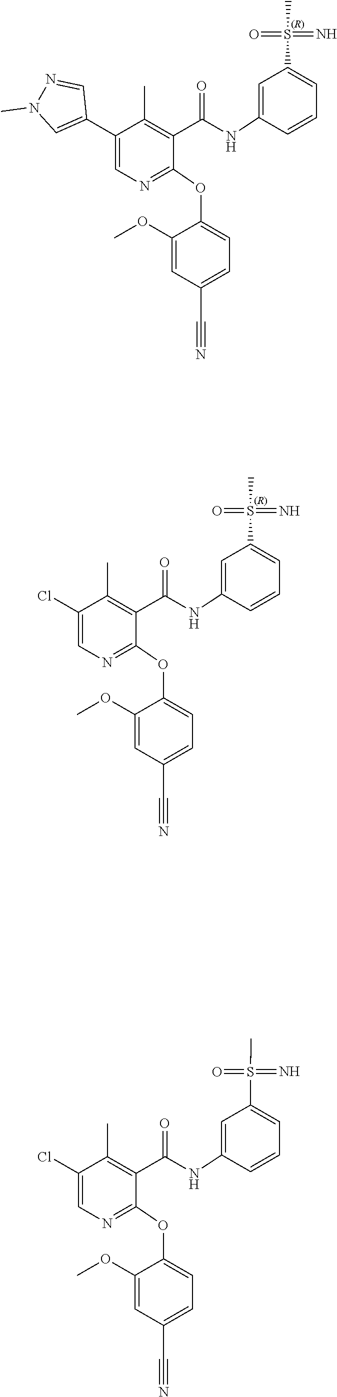

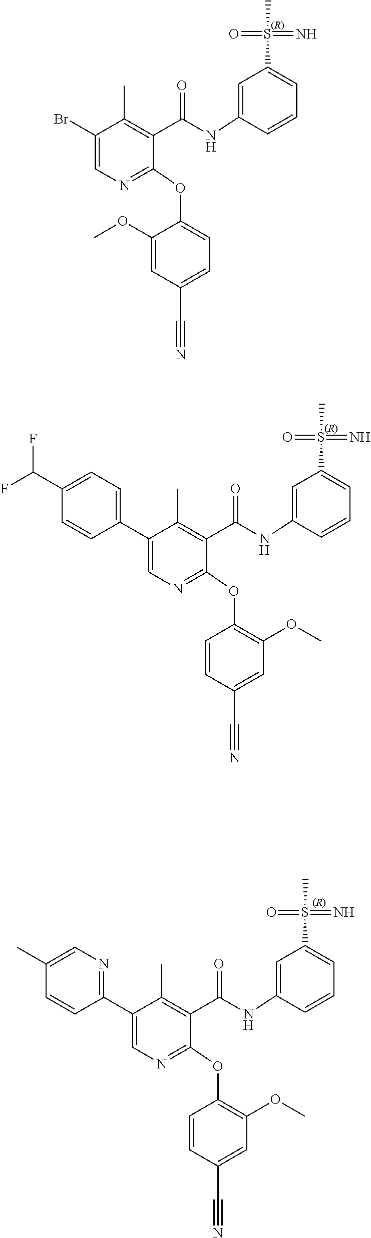

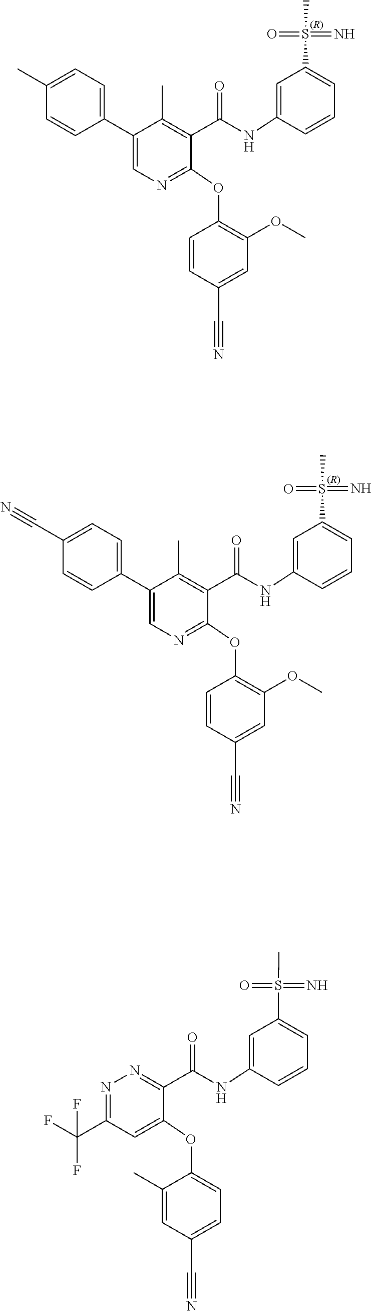

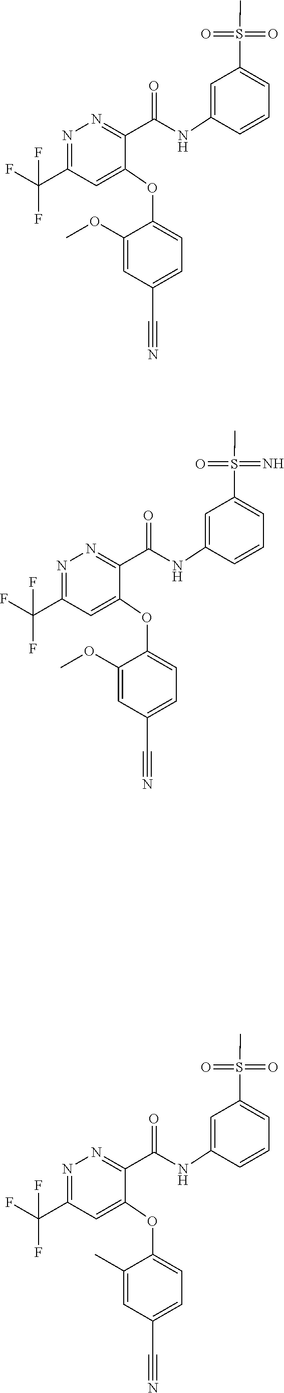 [SMILES: COc1cc(C#N)ccc1Oc1cc(C(F)(F)F)nnc1C(=O)Nc1cccc(S(C)(=O)=O)c1 ; COc1cc(C#N)ccc1Oc1cc(C(F)(F)F)nnc1C(=O)Nc1cccc(S(C)(=N)=O)c1 ; Cc1cc(C#N)ccc1Oc1cc(C(F)(F)F)nnc1C(=O)Nc1cccc(S(C)(=O)=O)c1]

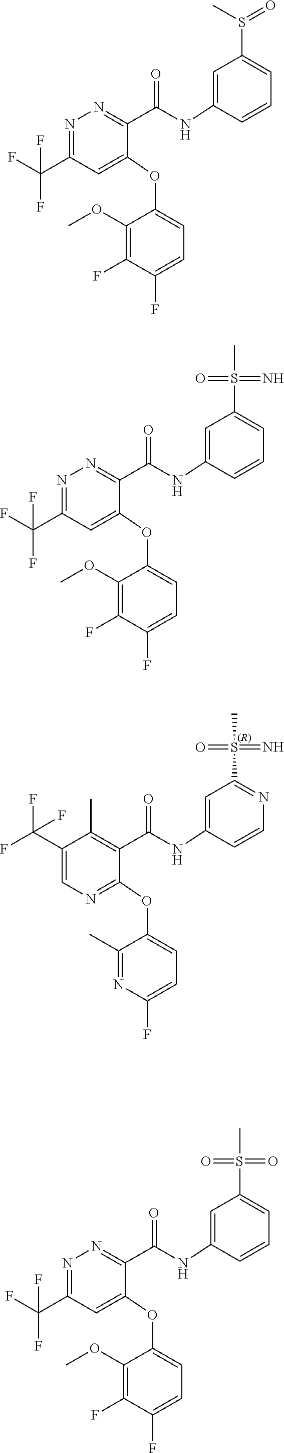

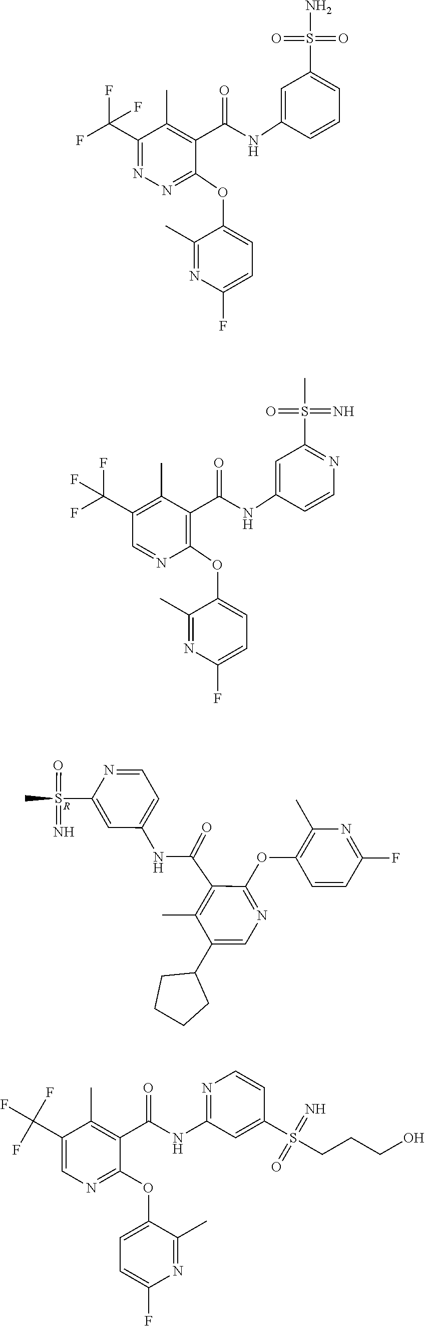

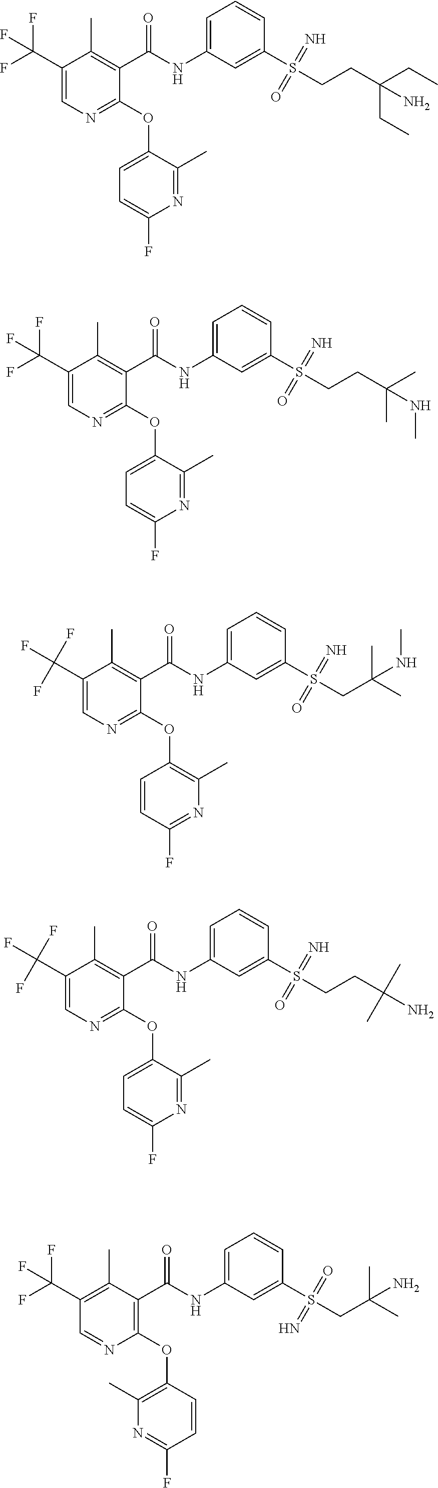 [SMILES: CCC(N)(CC)CCS(=N)(=O)c1cccc(NC(=O)c2c(Oc3ccc(F)nc3C)ncc(C(F)(F)F)c2C)c1 ; CNC(C)(C)CCS(=N)(=O)c1cccc(NC(=O)c2c(Oc3ccc(F)nc3C)ncc(C(F)(F)F)c2C)c1 ; CNC(C)(C)CS(=N)(=O)c1cccc(NC(=O)c2c(Oc3ccc(F)nc3C)ncc(C(F)(F)F)c2C)c1 ; Cc1nc(F)ccc1Oc1ncc(C(F)(F)F)c(C)c1C(=O)Nc1cccc(S(=N)(=O)CCC(C)(C)N)c1 ; Cc1nc(F)ccc1Oc1ncc(C(F)(F)F)c(C)c1C(=O)Nc1cccc(S(=N)(=O)CC(C)(C)N)c1]

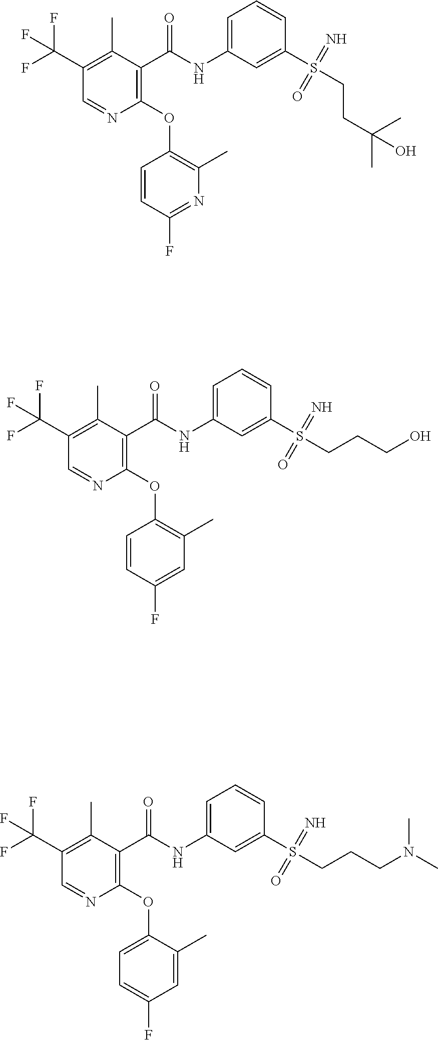 [SMILES: Cc1nc(F)ccc1Oc1ncc(C(F)(F)F)c(C)c1C(=O)Nc1cccc(S(=N)(=O)CCC(C)(C)O)c1 ; Cc1cc(F)ccc1Oc1ncc(C(F)(F)F)c(C)c1C(=O)Nc1cccc(S(=N)(=O)CCCO)c1 ; Cc1cc(F)ccc1Oc1ncc(C(F)(F)F)c(C)c1C(=O)Nc1cccc(S(=N)(=O)CCCN(C)C)c1]

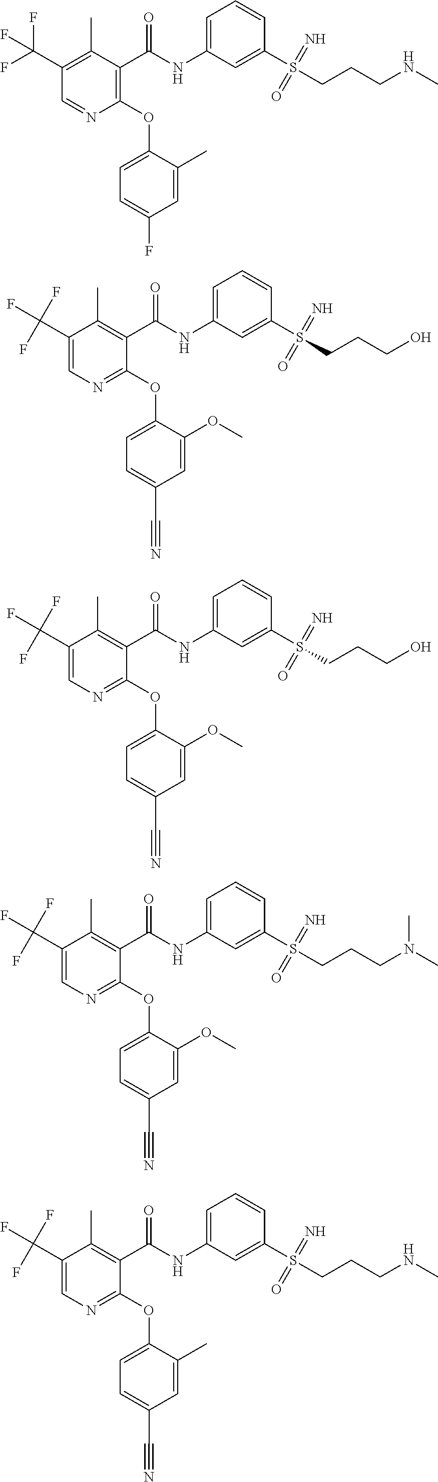

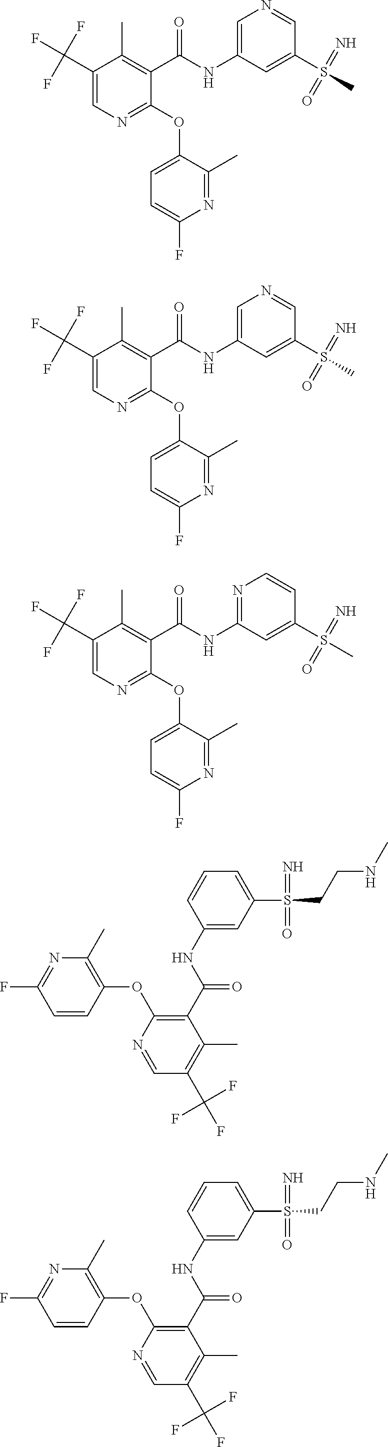 [SMILES: Cc1nc(F)ccc1Oc1ncc(C(F)(F)F)c(C)c1C(=O)Nc1cncc([S@](C)(=N)=O)c1 ; Cc1nc(F)ccc1Oc1ncc(C(F)(F)F)c(C)c1C(=O)Nc1cncc([S@@](C)(=N)=O)c1 ; Cc1nc(F)ccc1Oc1ncc(C(F)(F)F)c(C)c1C(=O)Nc1cc(S(C)(=N)=O)ccn1 ; CNCC[S@@](=N)(=O)c1cccc(NC(=O)c2c(Oc3ccc(F)nc3C)ncc(C(F)(F)F)c2C)c1 ; CNCC[S@](=N)(=O)c1cccc(NC(=O)c2c(Oc3ccc(F)nc3C)ncc(C(F)(F)F)c2C)c1]

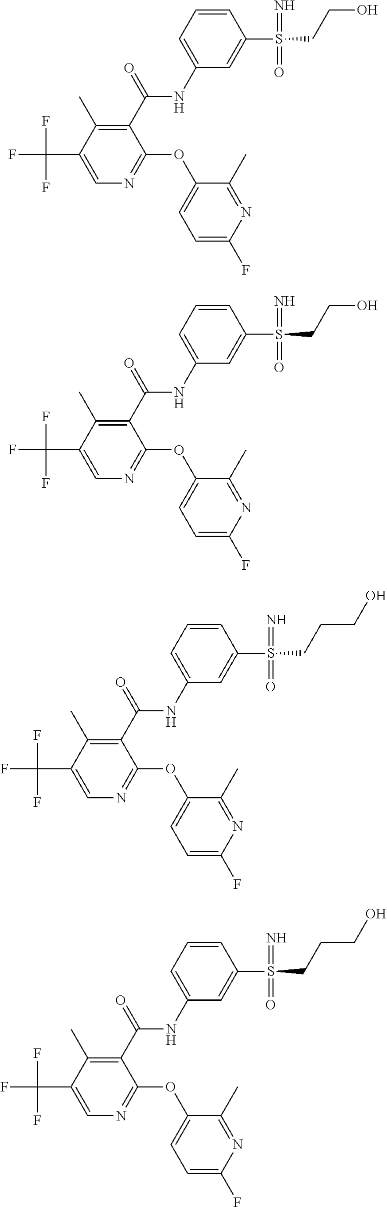

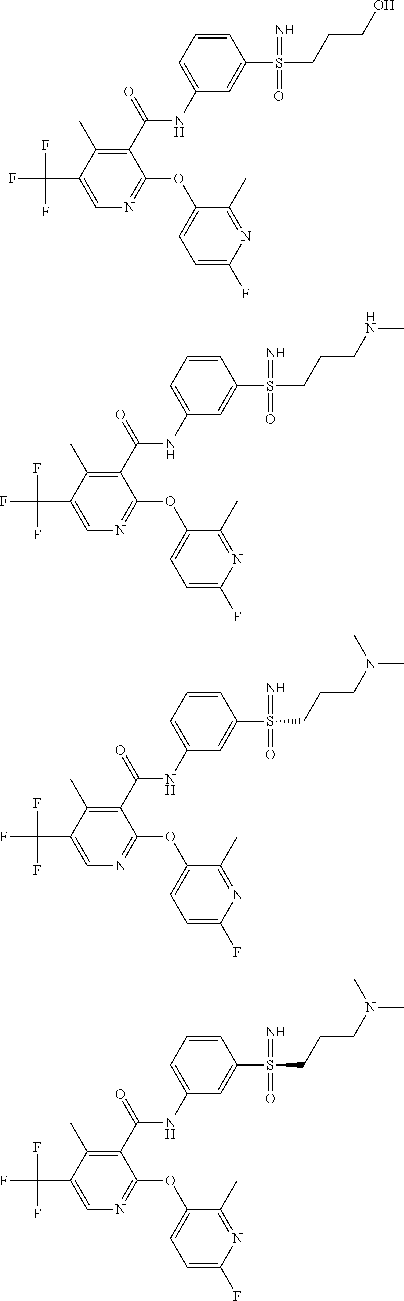 [SMILES: Cc1nc(F)ccc1Oc1ncc(C(F)(F)F)c(C)c1C(=O)Nc1cccc(S(=N)(=O)CCCO)c1 ; CNCCCS(=N)(=O)c1cccc(NC(=O)c2c(Oc3ccc(F)nc3C)ncc(C(F)(F)F)c2C)c1 ; Cc1nc(F)ccc1Oc1ncc(C(F)(F)F)c(C)c1C(=O)Nc1cccc([S@@](=N)(=O)CCCN(C)C)c1 ; Cc1nc(F)ccc1Oc1ncc(C(F)(F)F)c(C)c1C(=O)Nc1cccc([S@](=N)(=O)CCCN(C)C)c1]

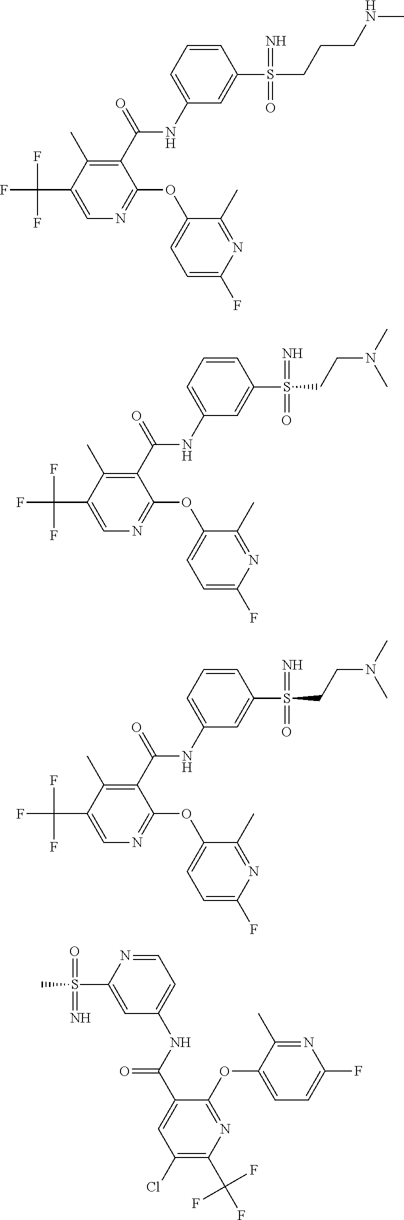

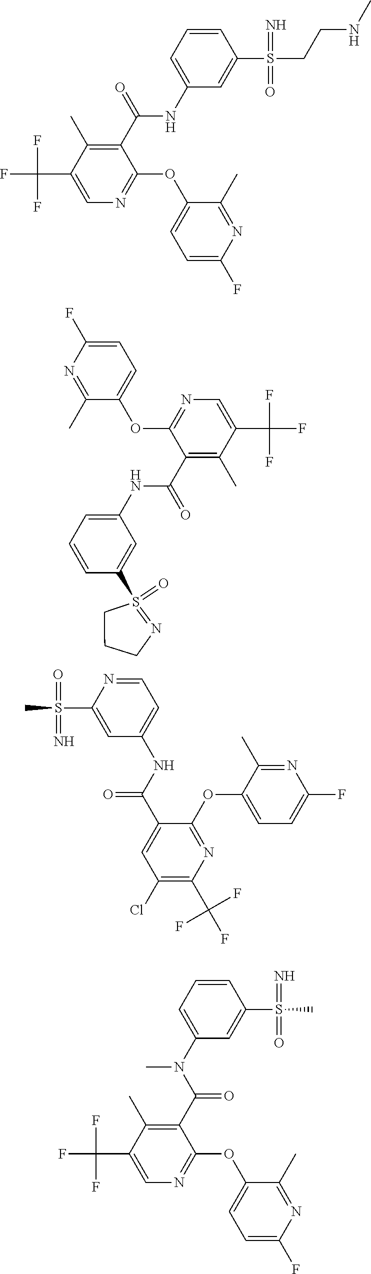

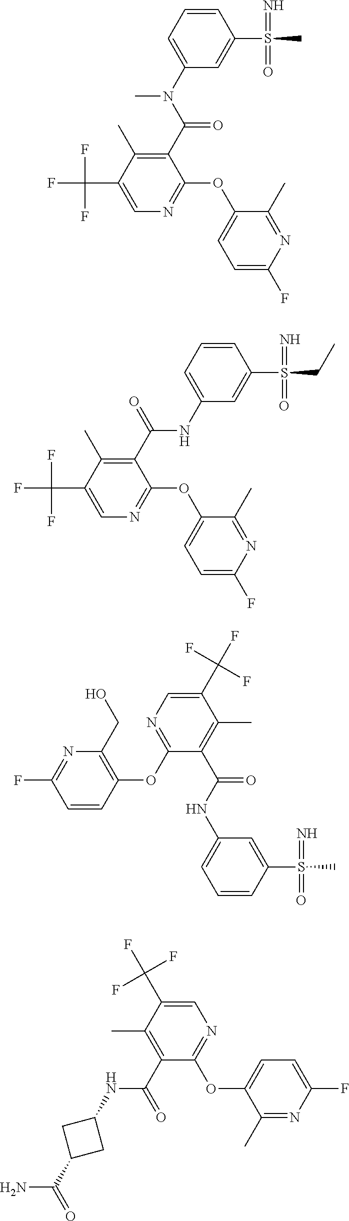

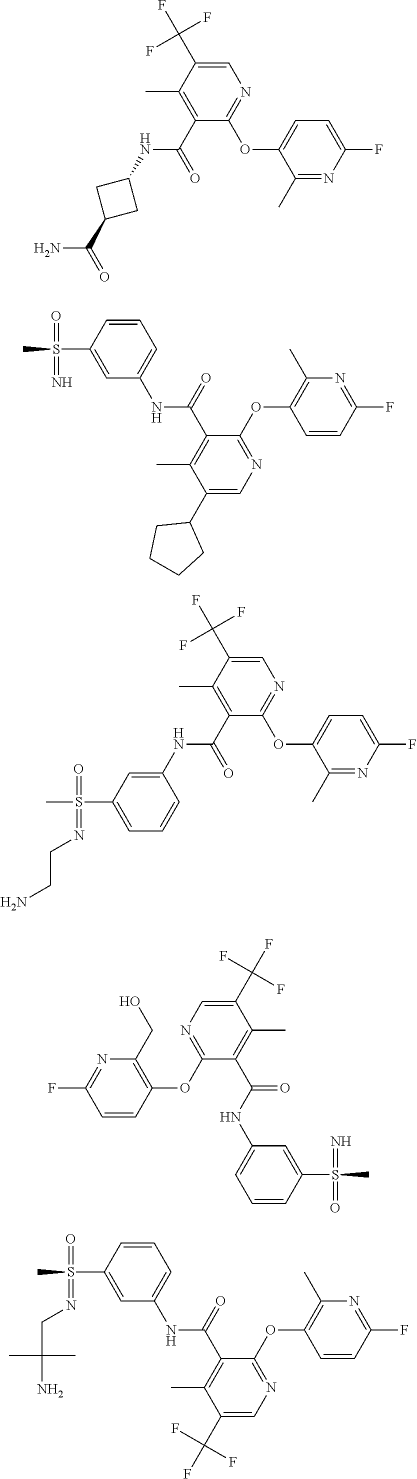

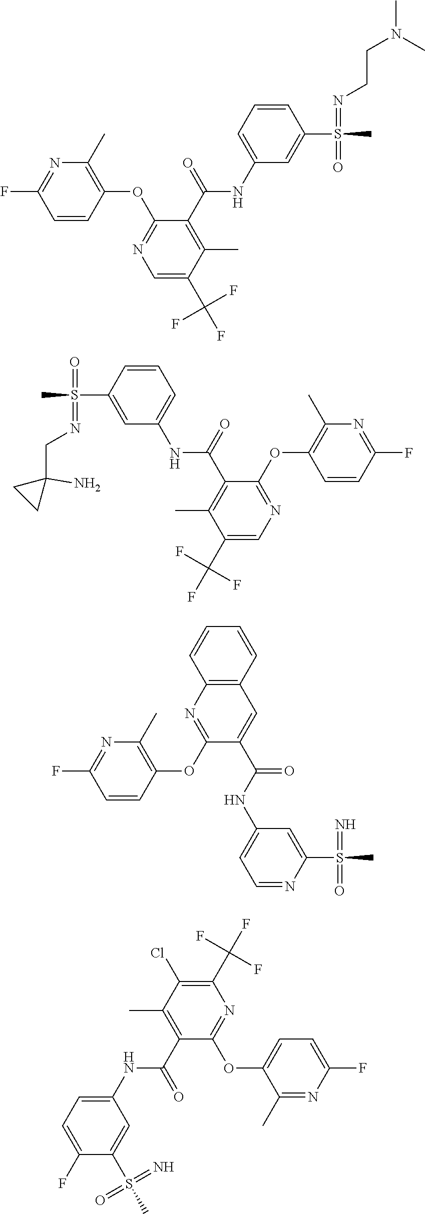

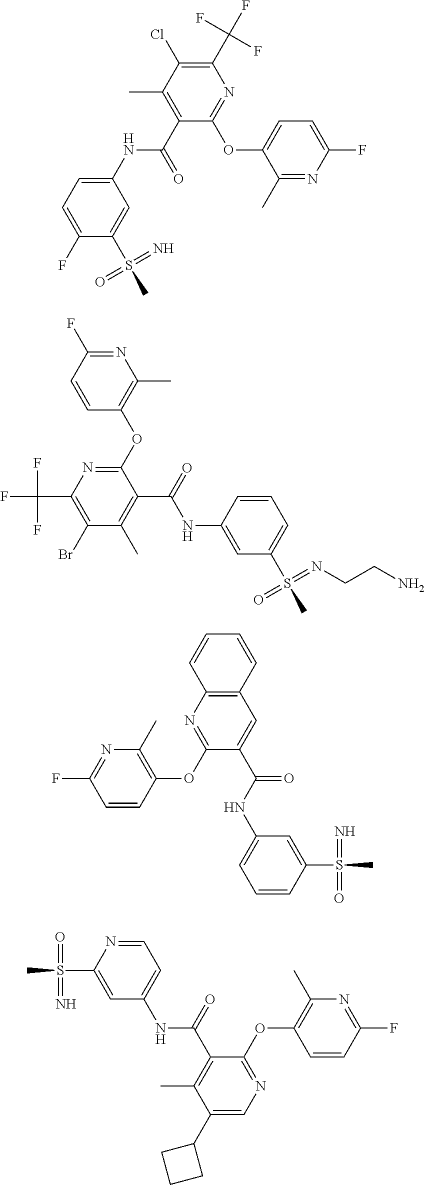

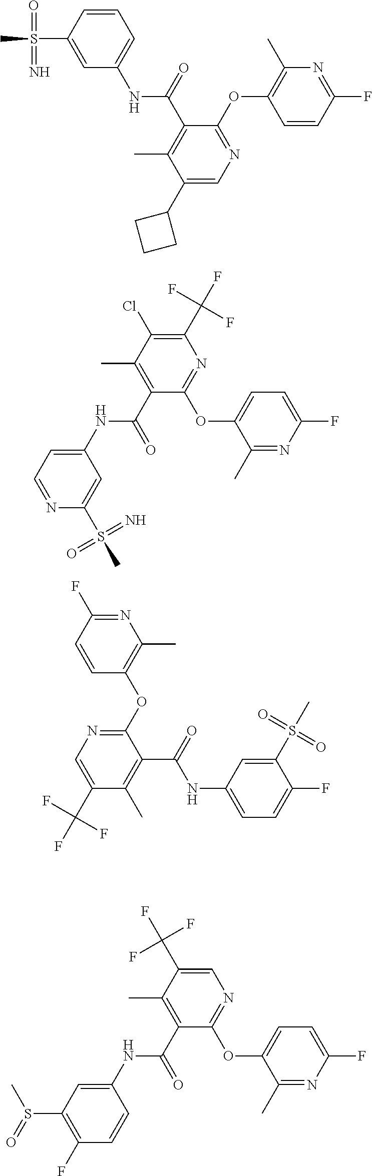

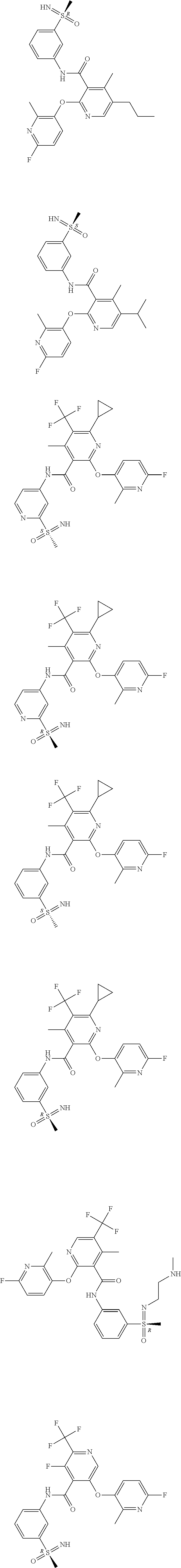

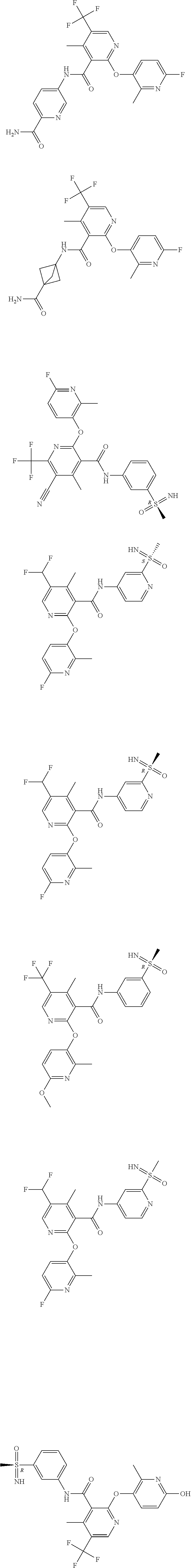

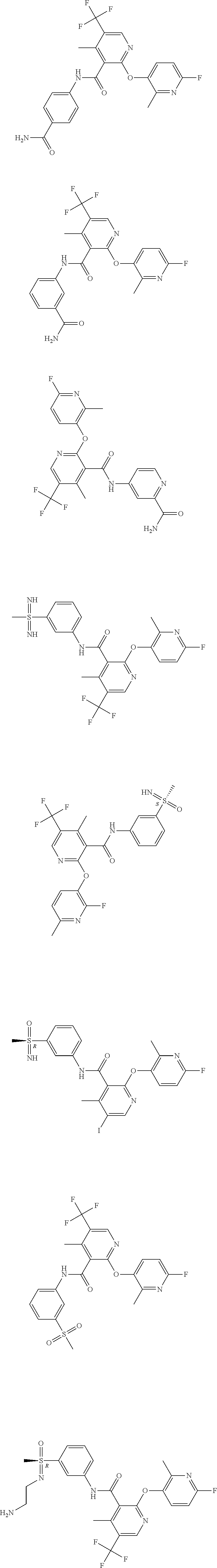

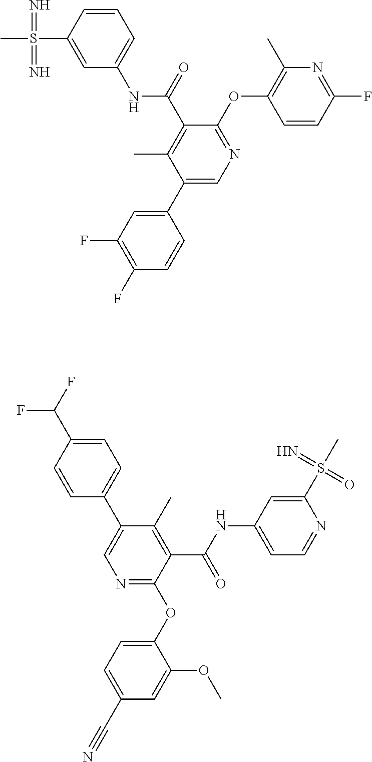 [SMILES: Cc1nc(F)ccc1Oc1ncc(-c2ccc(F)c(F)c2)c(C)c1C(=O)Nc1cccc(S(C)(=N)=N)c1 ; COc1cc(C#N)ccc1Oc1ncc(-c2ccc(C(F)F)cc2)c(C)c1C(=O)Nc1ccnc(S(C)(=N)=O)c1]

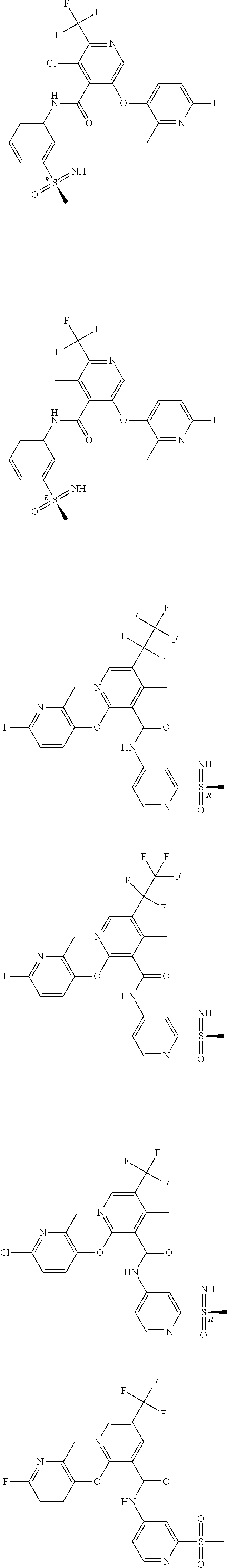

### (D) Fourth Set of Compounds

In one aspect, the invention provides a compound of Formula (I):

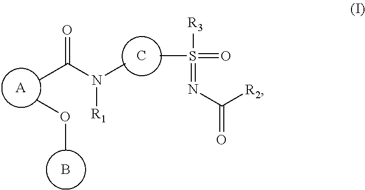

and pharmaceutically acceptable salts, hydrates and solvates thereof, wherein:
A is a substituted or unsubstituted heteroaryl ring comprising at least one heteroatom selected from a group consisting of O, S, or N;
wherein the one or more substitutions on A are selected from H, —OH, halo, C₁-C₈-alkyl, C₁-C₈ fully or partially fluorinated fluoroalkyl, C₂-C₈ branched alkyl, alkenyl, alkynyl, haloalkyl, alkoxy, cycloalkoxy, haloalkoxy, nitro, cyano, —C(R′)(R″)-cycloalkyl, C(R′)(R″)-aryl, —NR′R″, substituted or unsubstituted 3-8 membered cycloalkyl, 3-8 membered cycloalkenyl, or 3-6 membered heterocycloalkyl, wherein the 3-6 membered heterocycloalkyl comprises at least one heteroatom independently selected from O, S, and N;
B is substituted or unsubstituted aryl or heteroaryl, wherein the substitutions are selected from the group consisting of substituted or unsubstituted C₁-C₈ alkyl, deuterated C₁-C₄ alkyl wherein the alkyl chain may be fully or partially deuterated, halo C₁-C₄ alkyl where the alkyl chain is fully or partially halogenated, C₃-C₁₀ cycloalkyl, halogen, cyano, nitro, C₁-C₈ alkoxyl, haloalkoxyl wherein the haloalkyoxyl chain may be fully or partially halogenated, and arylalkoxyl;
C is substituted or unsubstituted aryl or heteroaryl, wherein the one or more substitutions are independently selected from a group consisting of halo, C₁-C₈ alkyl, haloalkyl, alkoxy;
R₁ is selected from a group consisting of: H and C₁-C₄ alkyl;
R₂ is selected from a group consisting of —(CH₂)ₙRₐ, —(CR_{b}R_{c})ₙRₐ, —(CR_{b}R_{c})ₙ—(OCH₂CH₂)ₙRₐ, —(CR_{b}R_{c})ₙ—(NR′CH₂CH₂)ₙRₐ, —(CR_{b}R_{c})ₙ—(NR′CH₂CH₂)ₙRₐ, and —(CR_{b}R_{c})ₙ—(NHCH₂CH₂NH)ₙRₐ;
wherein Rₐ is H, C₁-C₈-alkyl or branched alkyl, C₃-C₈ cycloalkyl, aryl, heteroaryl, 4-7 membered heterocyclyl, alkenyl, alkynyl, haloalkyl, OH, alkoxy, cycloalkoxy, haloalkoxy, —(OCH₂CH₂)ₙR_{b}, —NR′R″, —COONR′R″, —COOR′R″, alkylsulfonyl, arylsulfonyl, or —SO₂NR′R″;
R_{b} is H, F, C₁-C₆-alkyl, haloalkyl, branched alkyl, aryl, heteroaryl, 3-7 membered carbocyclyl or heterocyclyl with one or more heteroatoms;
R_{c} is H, C₁-C₆ alkyl, or F;
R_{c} and R_{b} together optionally form a 3-6 member carbocyclic or heterocyclic ring;
R₃ is selected from a group consisting of C₁-C₃ alkyl, C₃-C₄ cycloalkyl, haloalkyl, halocycloalkyl, aryl, heteroaryl, and heterocyclyl.

In another embodiment, the compound is a compound of Formula (II):

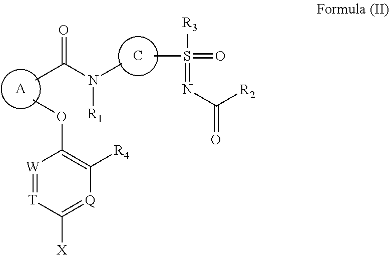

wherein: A, C, R₁, R₂, and R₃ are described above; Q, T and W are independently N or CR₅;
R₅ is H, halogen, —CD₃, alkyl, cycloalkyl, haloalkyl, alkoxy, haloalkoxy, cyano, —CF₃, —OCF₃, or cycloalkoxy;
R₄ is H, —OH, halo, —CD₃, C₁-C₆-alkyl, branched alkyl, haloalkyl where the alkyl chain is fully or partially halogenated, alkoxy, arylalkoxy, cycloalkoxy, haloalkoxy, cyano, —CH₂-cycloalkyl, —CH(CH₃)-cycloalkyl, trifluoromethyl, cyclopropylmethyl, 3-6 membered cycloalkyl or 3-6 membered heterocycloalkyl, any of which may have one or more substituents, wherein the 3-6 membered heterocycloalkyl comprises at least one heteroatom independently selected from O, S, and N;
X is H, halo, —CD₃, alkyl, haloalkyl, alkoxy, cycloalkoxy, haloalkoxy, cyano, —CF₃, or —OCF₃.

In another embodiment, the compound is a compound of Formula (III):

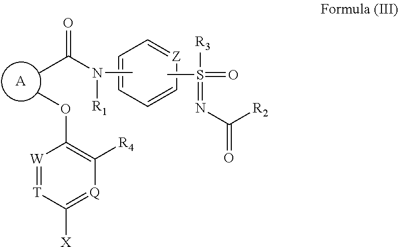

wherein, Z is CH, N, CF, or N⁺O⁻; and A, W, T, Q, X, R₁, R₂, R₃, and R₄ are described above.

In other embodiments, in the compounds of Formula (I), Formula (II), and/or Formula (III), ring A is substituted or unsubstituted 5-6 membered heteroaryl with one or more heteroatom. The 5-6 membered ring heteroaryl includes N as the one or more heteroatom. The heteroatom N may be in the form of an N-oxide, wherein the N-oxide is selected from a group consisting of: pyridyl N-oxide, pyrazinyl N-oxide, pyridazinyl N-oxide, and pyrimidinyl N-oxide.

In other embodiments, A is a 6-membered heteroaryl comprising at least one heteroatom, wherein the heteroatom is N. In certain embodiments, A is a 6-membered heteroaryl comprising 2 N atoms.

In other embodiments, in the compounds of Formula (I), Formula (II), and/or Formula (III), ring A may be a substituted or unsubstituted pyridyl, pyrimidinyl, pyrazinyl, or pyridazinyl.

In other embodiments, in the compounds of Formula (I), Formula (II), and/or Formula (III), ring A is.

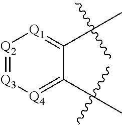

wherein Q₁, Q₂, Q₃ and Q₄ are independently selected from a group consisting of: N, N⁺O⁻, or CR₆; wherein at least two of Q₁, Q₂, Q₃ and Q₄ are CR₆; R₆ is H, —OH, halo, —CD₃, alkyl, haloalkyl, alkoxy, haloalkoxy, cyano, —CF₃, —OCF₃, substituted or unsubstituted 5 or 6 membered ring heterocyclyl or heteroaryl, saturated heterocyclyl, or partially unsaturated heterocyclyl, O-aryl, O-heteroaryl, O-cycloalkyl, or O-cycloheteroalkyl.

In other embodiments, in the compounds of Formula (I), Formula (II), and/or Formula (III), ring A is:

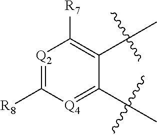

wherein,
Q₂ and Q₄ are independently N or N⁺O⁻;
Q₂ is N or N⁺O⁻ and Q₄ is CR₆; or
Q₂ is CR₆ and Q₄ is N or N⁺O⁻;
R₇ and R₈ are independently selected from the group consisting of H, —OH, halo, —CD₃, substituted or unsubstituted C₁-C₆ alkyl, branched alkyl, alkenyl, alkylnyl, haloalkyl, alkoxy, cycloalkyl, heterocyclyl, NH₂, NHR′, NR′R″, aryl, Heteroaryl, —CF₂CH₃, and —CF₂CF₃;
R₆ is described above.

In other embodiments, in the compounds of Formula (I), Formula (II), and/or Formula (III), ring A is:

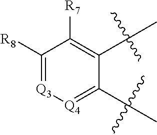

wherein,
Q₃ and Q₄ are N;
Q₃ is N, N⁺O⁻; Q₄ is CR₆; or
Q₃ is CR₆, Q₄ is N or N⁺O⁻; wherein R₇ and R₈ are defined above.

In other embodiments, in the compounds of Formula (I), Formula (II), and/or Formula (III), ring A is:

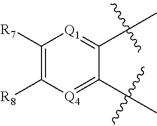

wherein
Q₁ and Q₄ are independently N or N⁺O⁻;
Q₁ is N or N⁺O⁻ and Q₄ is CR₆; or
Q₁ is CR₆ and Q₄ is N or N⁺O⁻; wherein R₆, R₇, and R₈ are described above.

In other embodiments, in the compounds of Formula (I), Formula (II), and/or Formula (III), ring A is:

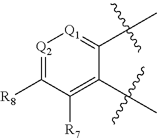

wherein Q₁ and Q₂ is N; wherein R₇ and R₈ are described above.

In other embodiments, in the compounds of Formula (I), Formula (II), and/or Formula (III), ring A is:

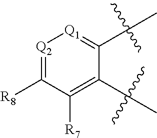

wherein Q₁ is CR₆, and Q₂ is N; R₆, R₇, and R₈ are described above.

In other embodiments, in the compounds of Formula (I), Formula (II), and/or Formula (III), ring A is:

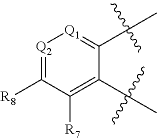

wherein, Q₁ is N; Q₂ is CR₆; R₆, R₇, and R₈ are described above.

In other embodiments, in the compounds of Formula (I), Formula (II), and/or Formula (III), R₁ is H.

In other embodiments, in the compounds of Formula (I), Formula (II), and/or Formula (III), R₃ is methyl.

In other embodiments, in the compounds of Formula (I), Formula (II), and/or Formula (III), R₂ is H, methyl, —CH₂—NH₂, —CH₂—NH—CH₃, —CH₂—OH, —CH(NH₂)(CH₃), —CH₂—OH, —CH(OH)(CH₃), —CH(CF₃)(NH₂), —CH₂—O—CH₃, amino-cycopropyl, pyrrolidine, azetidine, oxetane, tetrahydrofuran, or hydroxypyrrolidone.

In other embodiments, in the compounds of Formula (II) and/or Formula (III), T is N. In other embodiments, T is N, W is CH, and Q is CH.

In other embodiments, in the compounds of Formula (II) and/or Formula (III), R₄ is methyl. In other embodiments, in the compounds of Formula (II) and/or Formula (III), X is F.

In other embodiments, in the compounds of Formula (II) and/or Formula (III), Z is N or CH.

In other embodiments, the compound of the invention is selected from a group consisting of:

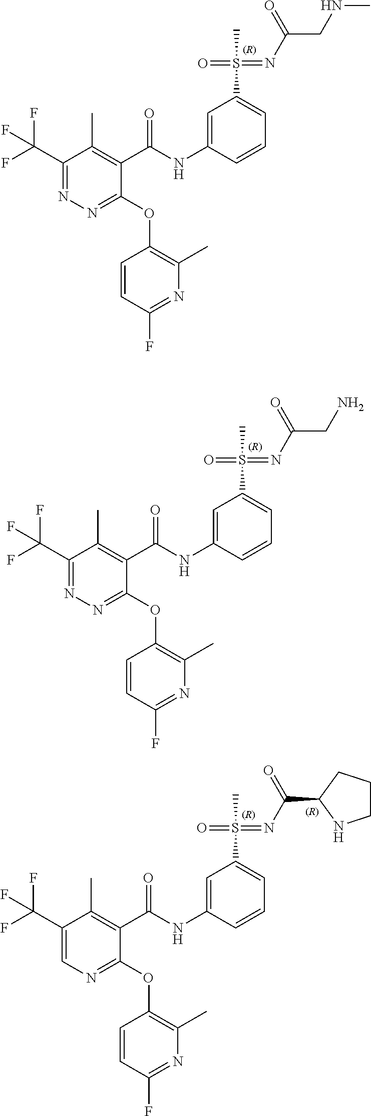

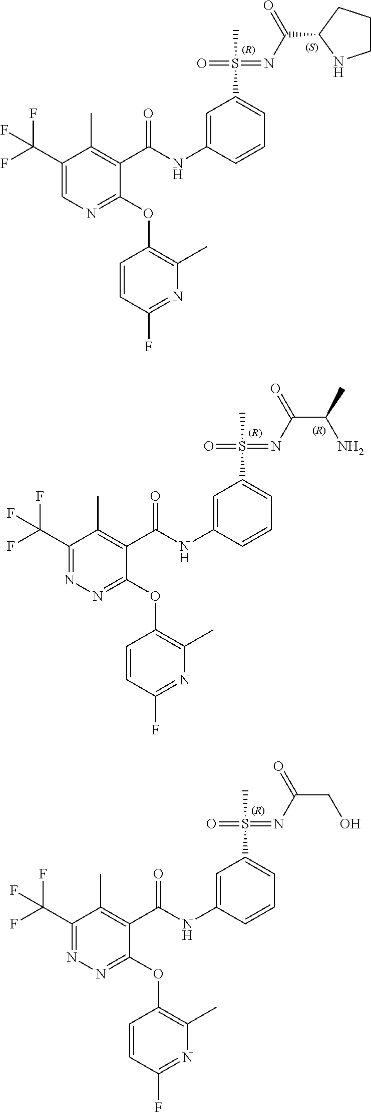

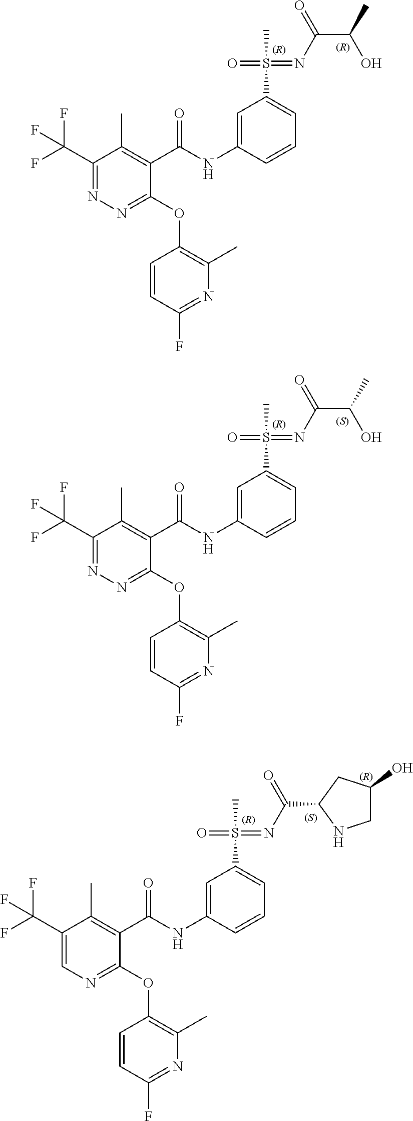

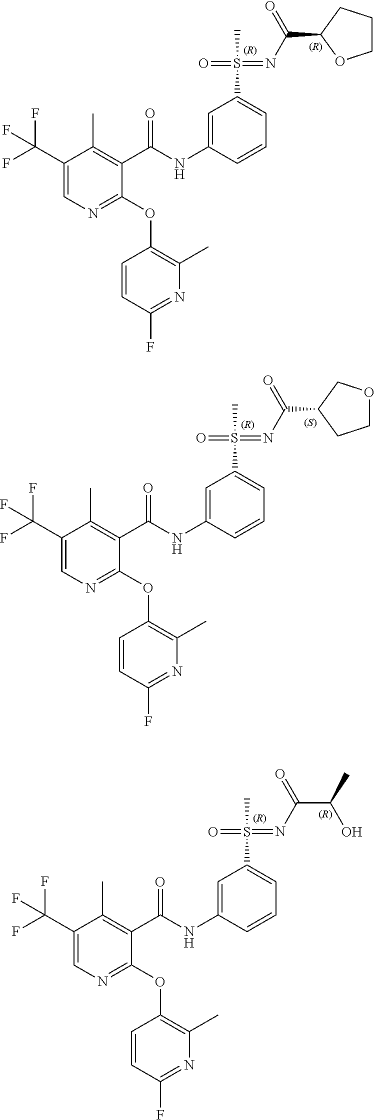

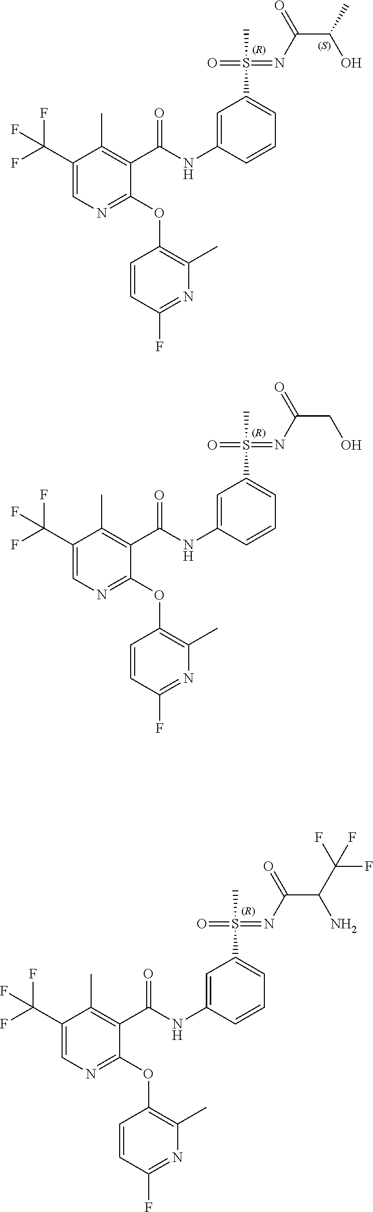

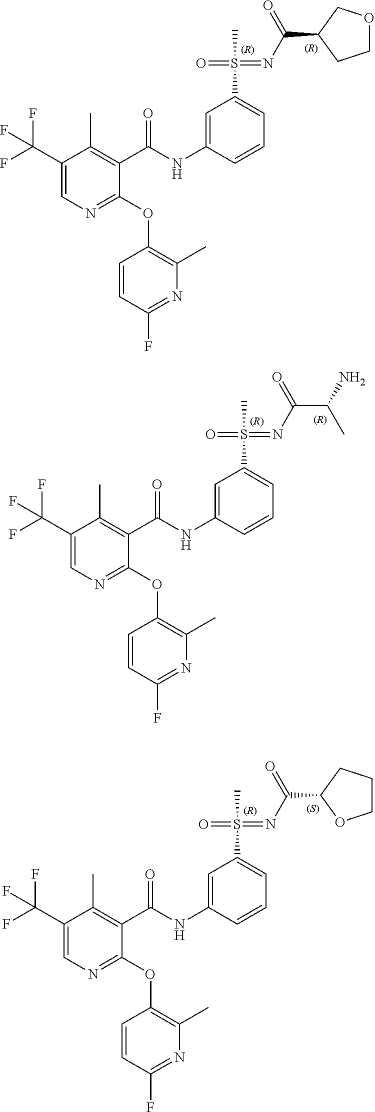

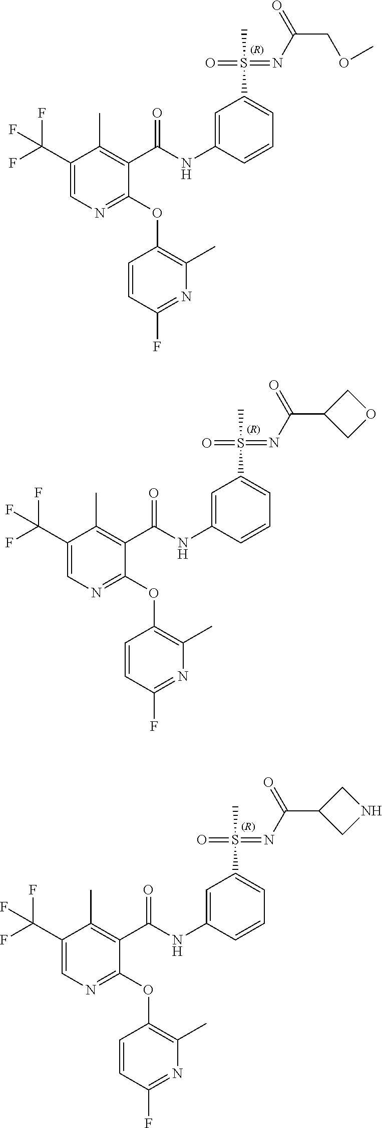

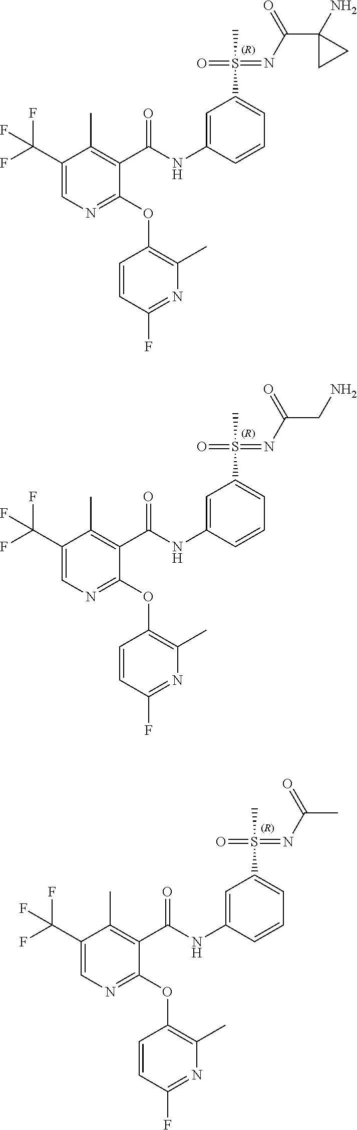

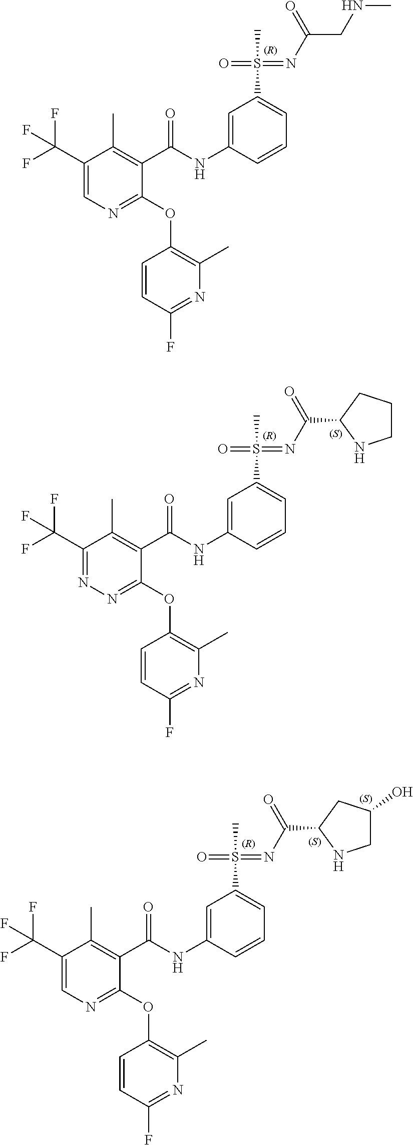

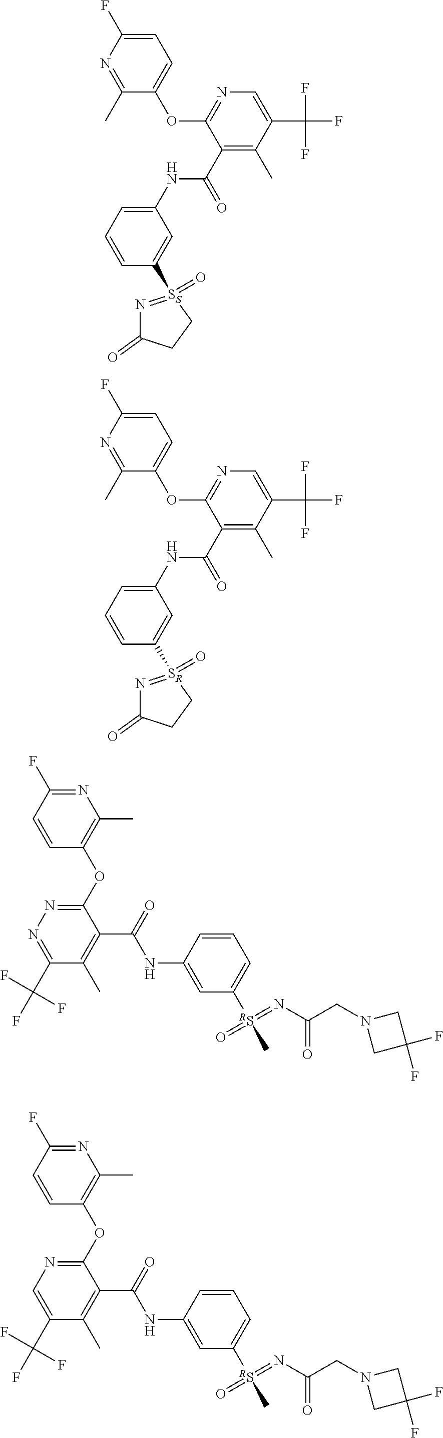

In another aspect, the invention provides inhibitors of a voltage gated sodium channel Nav1.8. The inhibitors may have a defined chemical structure, such as the structure of any of the compounds described above.

In another aspect, the invention provides methods of treating a condition in a subject by providing to a subject having a condition a compound of the invention, such as any of those described above.

The condition may be associated with aberrant activity of voltage gated sodium channels. The condition may be abdominal cancer pain, acute cough, acute idiopathic transverse myelitis, acute itch, acute pain, acute pain in major trauma/injury, airways hyperreactivity, allergic dermatitis, allergies, ankylosing spondylitis, asthma, atopy, Behcet [symbol] disease, bladder pain syndrome, bone cancer pain, brachial plexus injury, burn injury, burning mouth syndrome, calcium pyrophosphate deposition disease, cervicogenic headache, Charcot neuropathic osteoarthropathy, chemotherapy-induced oral mucositis, chemotherapy-induced peripheral neuropathy, cholestasis, chronic cough, chronic itch, chronic low back pain, chronic pain, chronic pancreatitis, chronic post-traumatic headache, chronic widespread pain, cluster headache, complex regional pain syndrome, complex regional pain syndromes, constant unilateral facial pain with additional attacks, contact dermatitis, cough, dental pain, diabetic neuropathy, diabetic peripheral neuropathy, diffuse idiopathic skeletal hyperostosis, disc degeneration pain, distal sensory polyneuropathy (DSP) associated with highly active antiretroviral therapy (HAART), Ehlers-Danlos syndrome, endometriosis, epidermolysis bullosa, epilepsy, erythromelalgia, Fabry [symbol] disease, facet joint syndrome, failed back surgery syndrome, familial hemiplegic migraine, fibromyalgia, glossopharyngeal neuralgia, glossopharyngeal neuropathic pain, gout, head and neck cancer pain, inflammatory bowel disease, inflammatory pain, inherited erythromelalgia, irritable bowel syndrome, irritable bowel syndrome, itch, juvenile idiopathic arthritis, mastocytosis, melorheostosis, migraine, multiple sclerosis, musculoskeletal damage, myofascial orofacial pain, neurodegeneration following ischemia, neurofibromatosis type II, neuropathic ocular pain, neuropathic pain, neuropathic pain, nociceptive pain, non-cardiac chest pain, optic neuritis, oral mucosal pain, orofacial pain, osteoarthritis, osteoarthritis, overactive bladder, pachyonychia congenita, pain, pain resulting from cancer, pain resulting from chemotherapy, pain resulting from diabetes, pain syndrome, painful joint arthroplasties, pancreatitis, Parkinson [symbol] disease, paroxysmal extreme pain disorder, pemphigus, perioperative pain, peripheral neuropathy, persistent idiopathic dentoalveolar pain, persistent idiopathic facial pain, phantom limb pain, phantom limb pain, polymyalgia rheumatica, postherpetic neuralgia, post-mastectomy pain syndrome, postoperative pain, post-stroke pain, post-surgical pain, post-thoracotomy pain syndrome, post-traumatic stress disorder, preoperative pain, pruritus, psoriasis, psoriatic arthritis, pudendal neuralgia, pyoderma gangrenosum, radiotherapy-induced peripheral neuropathy, Raynaud [symbol] disease, renal colic, renal colic, renal failure, rheumatoid arthritis, salivary gland pain, sarcoidosis, sciatica, scleroderma, sickle cell disease, small fiber neuropathy, spinal cord injury pain, spondylolisthesis, spontaneous pain, stump pain, subacute cough, temporomandibular joint disorders, tension-type headache, trigeminal neuralgia, vascular leg ulcers, vulvodynia, or whiplash associated disorder. In another aspect, the invention provides methods of making a medicament using a compound of the invention, such as any of those described above.

In another aspect the invention provides products comprising a compound of the invention, such as any of those described above, for treatment of a condition, such as any of those described above, in a subject.

### IV. DETAILED DESCRIPTION

### Definitions

Unless otherwise defined, all technical and scientific terms used herein have the same meaning as commonly understood by one of ordinary skill in the art to which this presently described subject matter belongs. The definitions provided below are intended to supplement and illustrate, not preclude, the definitions that would be apparent to one of ordinary skill in the art upon review of the present disclosure.

Unless otherwise stated, the moieties described below are optionally substituted, i.e., they may be substituted at one or more positions. The terms substituted, whether preceded by the term “optionally” or not, and substituent, as used herein, refer to the ability to change one or more functional groups for another functional group or groups on a molecule, provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted at one or more positions).

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless otherwise explicitly defined, a “substituent group,” as used herein, includes a functional group selected from one or more of the following moieties, which are defined herein.

The term hydrocarbon, as used herein, refers to any chemical group comprising hydrogen and carbon. The hydrocarbon may be substituted or unsubstituted. As would be known to one skilled in the art, all valences must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, isopropyl, cyclopropyl, ally 1, vinyl, n-butyl, tert-butyl, ethynyl, cyclohexyl, and the like.

The term “alkyl” by itself or as part of another substituent, means, unless otherwise stated, a straight (i.e., unbranched) or branched chain, acyclic or cyclic saturated hydrocarbon group, or combination thereof, and can include di- and multivalent groups, having the number of carbon atoms designated (e.g., C₁₋₁₀ means one to ten carbons, including 1, 2, 3, 4, 5, 6, 7, 8, 9, and 10 carbons). In particular embodiments, the term “alkyl” refers to C₁₋₂₀ inclusive, including 1, 2, 3, 4, 5, 6, 7, 8, 9, 10, 11, 12, 13, 14, 15, 16, 17, 18, 19, and 20 carbons, linear (i.e., “straight-chain”), branched, or cyclic saturated hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologues and isomers thereof.

“Branched” refers to an alkyl group in which a lower alkyl group, such as methyl, ethyl, or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, cyano, and mercapto.

The term “heteroalkyl,” by itself or in combination with another term, means, unless otherwise stated, a stable straight or branched chain having from 1 to 20 carbon atoms or heteroatoms or a cyclic hydrocarbon group having from 3 to 15 carbon atoms or heteroatoms, or combinations thereof, consisting of at least one carbon atom and at least one heteroatom, such as O, N, P, Si or S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)NR′, —NR′R″, —OR′, —SR, —S(O)R, and/or —S(O₂)R′.

“Cycloalkyl” refers to a saturated monocyclic or multicyclic ring system of from about 3 to about 15 carbon atoms, e.g., 3, 4, 5, 6, 7, 8, 9, or 10 carbon atoms. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, unsubstituted alkyl, substituted alkyl, aryl, or substituted aryl, thus providing a heterocyclic group. Examples of cycloalkyl include, but are not limited to, cyclopentyl, cyclohexyl, 1-cyclohexenyl, 3-cyclohexenyl, cycloheptyl, and the like.

The term “cycloalkylalkyl,” as used herein, refers to a cycloalkyl group as defined above, which is attached to the parent molecular moiety through an alkylene moiety, also as defined above, e.g., a C₁₋₂₀ alkylene moiety. Examples of cycloalkylalkyl groups include cyclopropylmethyl and cyclopentylethyl.

The term “carbocyclyl” refers to a monocyclic or multicyclic ring system of from about 3 to about 15 ring members in which all ring members are carbon atoms. Unless otherwise specified, a carbocyclyl may be saturated, partially saturated (i.e., have one or more double or triple bonds), or aromatic.

The term “heterocyclyl” refers to a monocyclic or multicyclic ring system of from about 3 to about 15 ring members in which at least one ring member is a heteroatom, such as N, O, or S. Unless otherwise specified, a heterocyclyl may be saturated, partially saturated (i.e., have one or more double or triple bonds), or aromatic. Examples of saturated and partially unsaturated non-aromatic heterocyclic groups include, but are not limited to, 3-oxetanyl, 2-oxetanyl, azetidinyl, thietanyl, tetrahydrofuranyl, tetrahydrothiophenyl, pyrrolidinyl, dihydropyranyl, tetrahydropyranyl, thio-dihydropyranyl, thio-tetrahydropyranyl, piperidinyl, piperazinyl, morpholinyl, 1,3-oxazinanyl, 1,3-thiazinanyl, 4,5,6-tetrahydropyrimidinyl, 2,3-dihydrofuranyl, dihydrothienyl, dihydropyridinyl, tetrahydropyridinyl, isoxazolidinyl, pyrazolidinyl, tetrazolyl, imidazolyl, isothiozolyl, triazolyl, azabicyclo-octanyl, diazabicyclo-octanyl, and all alkyl, alkoxy, haloalkyl and haloalkoxy substituted derivatives of any of the aforementioned groups.

The terms “cycloheteroalkyl” and “heterocycloalkyl” refer to a saturated ring system, such as a 3- to 10-member cycloalkyl ring system, that include one or more heteroatoms. The heteroatoms may be the same or different and may be nitrogen (N), oxygen (O), or sulfur (S). Examples of heterocycloalkyl include, but are not limited to, 1-(l, 2,5,6-tetrahydropyridyi), 1-piperidmyl, 2-piperidinyl, 3-piperidinyl, 4-morpholinyl, 3-morpholinyl, tetrahydrofuran-3-yl, tetrahydrofuran-3-yl, tetrahydrothien-2-yl, tetrahydrothien-3-yl, 1-piperazinyl, 2-piperazinyl, and the like.

The cycloheteroalkyl ring can be optionally fused to or otherwise attached to other cycloheteroalkyl rings and/or non-aromatic hydrocarbon rings. Heterocyclic rings include those having from one to three heteroatoms, such as oxygen, sulfur, and nitrogen, in which the nitrogen and sulfur heteroatoms may optionally be oxidized, and the nitrogen heteroatom may optionally be quaternized. Examples include, but are not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidinyl, piperazinyl, indolinyl, quinuclidinyl, morpholinyl, thiomorpholinyl, thiadiazinanyl, tetrahydrofuranyl, and the like.

An unsaturated hydrocarbon, carbocyclyl, or heterocyclyl has one or more double bonds or triple bonds. Examples of unsaturated hydrocarbons include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “alkenyl” as used herein refers to a monovalent group derived from a C₂₋₂₀ inclusive straight or branched hydrocarbon moiety having at least one carbon-carbon double bond by the removal of a single hydrogen molecule. Alkenyl groups include, for example, ethenyl (i.e., vinyl), propenyl, butenyl, 1-methyl-2-buten-1-yl, pentenyl, hexenyl, octenyl, allenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₂₋₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, and heptynyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched, or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (CH₂)₃, cyclohexylene (—C₆H₁₀—, —CH═CH—CH═CH—, —CH═CH—CH₂—, —CH₂CH₂CH₂CH₂CH₂—, —CH₂CH₂CH(CH₂CH₂CH₃)CH₂—, —(CH₂)q-N(R)—(CH₂)r-, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—).

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms also can occupy either or both of the chain termini (e.g., alkyleneoxo, alkylenedioxo, alkyleneamino, alkylenediamino, and the like).

Still further, for alkylene and heteroalkylene linking groups, no orientation of the linking group is implied by the direction in which the formula of the linking group is written. For example, the formula —C(O)OR′— represents both —C(O)OR′— and —R′OC(O)—.

The term “spirocyclyl” refers to a polycyclic compound in which two rings have a single atom, e.g., carbon, as the only common member of two rings. Thus, a “spirocycloalkyl” refers to a cycloalkyl group with two rings having a single carbon in common, and a “spiroheterocycloalkyl” or “spiroheterocycloalkyl” refers to a cycloheteroalkyl group with two rings having a single carbon or other atom, e.g., nitrogen, in common.

The term “aryl” means, unless otherwise stated, an aromatic hydrocarbon substituent that can be a single ring or multiple rings (such as from 1 to 3 rings), which are fused together or linked covalently.

The term “heteroaryl” refers to and groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyndyl, 4-pyridyl, 2-pyrimidyl, 4-pyrimidyl, 5-benzoihiazolyl, purinyl, 2-benzimidazolyl, 5-indolyl, 1-isoquinolyl, 5-isoquinolyl, 2-quinoxalinyl, 5-quinoxalinyl, 3-qumolyl, and 6-quinolyl.

Substituents for each of above noted aryl and heteroaryl ring systems are selected from the group of acceptable substituents described below. The terms “arylene” and “heteroarylene” refer to the divalent forms of aryl and heteroaryl, respectively.

Where a heteroalkyl, heterocycloalkyl, or heteroaryl includes a specific number of members (e.g., “3 to 7 membered”), the term “member” refers to a carbon atom or heteroatom.

Each of the above terms is meant to include both substituted and unsubstituted forms of the indicated group. Optional substituents are provided below.

Substituents can be one or more of a variety of groups selected from, but not limited to: —OR′, ═O, ═NR′, ═N—OR′, —NR′R″—SR′, -halogen, —SiR′R″R″, —OC(O)R, —C(O)R, —CO₂R—C(O)NR′R″, —OC(O)NR′R″, —NR″C(O)R, —NR′—C(O)NR″R′″, —NR″C(O)OR′, —NR—C(NR′R″)═NR′″, —S(O)R, —S(O)₂R′, —S(O)₂NR′R′, —NRSO₂R′, —CN, CF₃, fluorinated C₁₋₄ alkyl, and —NO₂ in a number ranging from zero to (2m′+1), where m′ is the total number of carbon atoms in such groups. R′, R″, R′″ and R″″ each may independently refer to hydrogen, halogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups.

Other non-limiting examples of substituents include (C₁-C₆)alkyl, (C₂-C₅)alkenyl, (C₃-C₅)alkynyl, halogen, halo(C₁-C₆)alkyl, hydroxy, —O(C₁-C₆)alkyl, halo(C₁-C₆)alkoxy, (C₃-C₅)cycloalkyl, (C₆-C₁₀)aryl, heterocyclyl, heteroaryl, amino, cyano, nitro, (C₁-C₆)alkyl-OH, (C₁-C₆)alkyl-NH₂, (C₁-C₆)alkyl-O—(C₁-C₆)alkyl, (C₁-C₆)alkyl-N—(C₁-C₆)alkyl, (C₁-C₆)alkyl(C₆-C₁₀)aryl, —C(O)(C₁-C₆)alkyl, —C(O)NR′R″, —S(O)(C₁-C₆)alkyl, —S(O)NR′R″, —S(O)₂(C₁-C₆)alkyl, —S(O)₂NR′R″, —O(C₁-C₆)alkyl-S(O)(C₁-C₆)alkyl, —O(C₁-C₆)alkyl-S(O)NR′R″, —O(C₁-C₆)alkyl-S(O)₂(C₁-C₆)alkyl, and —O(C₁-C₆)alkyl-S(O)₂NR′R″. As used herein, an “alkoxy” group is an alkyl attached to the remainder of the molecule through a divalent oxygen.

When a compound of the disclosure includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of ordinary skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Two of the substituents on adjacent atoms of aryl or heteroaryl ring may optionally form a ring of the formula -T-C(O)—(CRR′)q-U-, wherein T and U are independently —NR—, —O—, —CRR′— or a single bond, and q is an integer from 0 to 3. Alternatively, two of the substituents on adjacent atoms of aryl or heteroaryl ring may optionally be replaced with a substituent of the formula -A-(CH₂)r-B—, wherein A and B are independently —CRR′—, —O—, —NR—, —S—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)s-X′—(C″R′″)d-, where s and d are independently integers of from 0 to 3, and X′ is —O—, —NR′—, —S—, —S(O)—, —S(O)₂—, or —S(O)₂NR′—.

The substituents R, R′, R″ and R″ may be independently selected from halogen, hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocyclic, heterocyclic, or aromatic heterocyclic group as defined herein). As such, the term “acyl” specifically includes aryl acyl groups, such as a 2-(furan-2-yl)acetyl)- and a 2-phenylacetyl group. Specific examples of acyl groups include acetyl and benzoyl. Acyl groups also are intended to include amides, —RC(═O)NR, esters, —RC(═O)OR′, ketones, —RC(═O)R′, and aldehydes, —RC(═O)H.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxy alkyl” as used herein refers to an alkyl-O-alkyl ether, for example, a methoxy ethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described and includes substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplar) aralkyloxyl group is benzyloxyl, i.e., C₆H₅CH₂—O—. An aralkyloxyl group can optionally be substituted.

“Alkoxycarbonyl” refers to an alkyl-O—C(═O)— group. Exemplary alkoxy carbonyl groups include methoxycarbonyl, ethoxy carbonyl, butyloxycarbonyl, and tert-butyloxycarbonyl.

“Aryloxycarbonyl” refers to an aryl-O—C(═O)— group. Exemplary aryloxy carbonyl groups include phenoxy- and naphthoxy-carbonyl.

“Aralkoxycarbonyl” refers to an aralkyl —O—C(═O)— group. An exemplary aralkoxycarbonyl group is benzyloxycarbonyl.

“Carbamoyl” refers to an amide group of the formula —C(═O)NH₂.

“Alkylcarbamoyl” refers to a R′RN—C(═O) group wherein one of R and R′ is hydrogen and the other of R and R′ is alkyl and/or substituted alkyl as previously described.

“Dialkylcarbamoyl” refers to a R′RN—C(═O)— group wherein each of R and R′ is independently alkyl and/or substituted alkyl as previously described.

The term “carbonyldioxyl,” as used herein, refers to a carbonate group of the formula —OC(═O)—OR.

“Acyloxyl” refers to an acyl-O— group wherein acyl is as previously described.

The term “amino” refers to the —NH₂ group and refers to a nitrogen containing group as is known in the art derived from ammonia by the replacement of one or more hydrogen radicals by organic groups. For example, the terms “acyl amino” and “alkylamino” refer to specific N-substituted organic groups with acyl and alkyl substituent groups respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R′″ are each independently selected from the group consisting of alkyl groups. Additionally, R′, R″, and/or R′″ taken together may optionally be —(CH₂)ₖ where k is an integer from 2 to 6. Examples include, but are not limited to, methylamino, dimethylamino, ethylamino, diethylamino, diethylaminocarbonyl, methylethylamino, isopropyl amino, piperidino, trimethylamino, and propylamine.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to the —C(═O)— group, and can include an aldehyde group represented by the general formula R—C(═O)H.

The term “carboxyl” refers to the COOH group. Such groups also are referred to herein as a “carboxylic acid” moiety.

The term “cyano” refers to the —CN group.

The terms “halo,” “halide,” and “halogen” refer to fluoro, chloro, bromo, and iodo groups.

The term “haloalkyl” refers to an alkyl group substituted with one or more halogens. Additionally, the term “haloalkyl,” includes monohaloalkyl and polyhaloalkyl. For example, the term “halo(C₁₋₄)alkyl” includes, but is not limited to, trifluoromethyl, 2,2,2-trifluoroethyl, 4-chlorobutyl, 3-bromopropyl, and the like.

The terms “halocycloalky” and “cyclohaloalkyl” refer to a cycloalkly group with one or more halogens.

The term “hydroxyl” refers to the —OH group.

The term “hydroxy alkyl” refers to an alkyl group substituted with an —OH group.

The term “mercapto” refers to the —SH group.

The term “oxo” refers to an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

More particularly, the term “sulfide” refers to compound having a group of the formula —SR.

The term “sulfone” refers to compound having a sulfonyl group —S(O₂)R′.

The term “sulfoxide” refers to a compound having a sulfinyl group —S(O)R

The term ureido refers to a urea group of the formula —NH—CO—NH₂.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisomeric forms that may be defined, m terms of absolute stereochemistry, as (R)- or (S)- or, as D- or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)-, or D- and L-isomers may be prepared using chiral synthons or chiral reagents or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure, i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium, and which are readily converted from one isomeric form to another.

Unless otherwise stated, structures depicted herein are also meant to include compounds which differ only in the presence of one or more isotopically enriched atoms. For example, compounds having the present structures with the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example, tritium (³H), iodine-125 (¹²⁵I) or carbon-14 (¹⁴C). All isotopic variations of the compounds of the present disclosure, whether radioactive or not, are encompassed within the scope of the present disclosure.

The compounds of the present disclosure may exist as salts, and particularly as pharmaceutically acceptable salts. The present disclosure includes such salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures, succinates, benzoates, and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or m a suitable inert solvent or by ion exchange. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure contain both basic and acidic functionalities that allow □the compounds to be converted into either base or acid addition salts.

The neutral forms of the compounds may be regenerated by contacting the salt with a base or acid and isolating the parent compound in the conventional manner.

The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

In addition to salt forms, the present disclosure provides compounds that are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

The term “protecting group” refers to chemical moieties that block some or all reactive moieties of a compound and prevent such moieties from participating in chemical reactions until the protective group is removed, for example, those moieties listed and described in T. W. Greene, P. G. M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups.

For example, protective groups can be removed by acid, base, and hydrogenolysis. Groups such as trityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as tert-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be blocked with oxidatively-removable protective groups such as 2,4-dimethoxybenzyl, while co existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(O)-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

### Abbreviations and Acronyms

When the following abbreviations are used herein, they have the following meaning:
Ac2O acetic anhydride
anhy Anhydrous
n-BuOH n-butanol
t-BuOH t-butanol
CD₃OD methanol-d₄
Celite® diatomaceous earth filter agent, ® Celite Corp.
CH₂Cl₂ methylene chloride
DCM dichloromethane
CI-MS chemical ionization mass spectroscopy
conc concentrated
dec decomposition
bs broad singlet
br broad
DME dimethoxyethane
DMF N,N-dimethylformamide
DMSO dimethylsulfoxide
DMSO-d₆ dimethylsulfoxide-d₆
ELSD evaporative light scattering device
EtOAc ethyl acetate
EtOH ethanol (100%)
Et₂O diethyl ether
Et₃N triethylamine
HPLC ESI-MS high performance liquid chromatography-electrospray mass spectroscopy
MPLC medium pressure liquid chromatography
NMR nuclear magnetic resonance spectroscopy
TOF-MS time-of-flight-mass spectroscopy
NMM 4-methylmorpholine
Ph₃P triphenylphosphine
Pd(dppf)Cl₂ [1,1′-bis(diphenylphosphino) ferrocene]dichloropalladium (II)
Pd(PPh₃)₄ tetrakis(triphenylphosphine)palladium(0)
Pd(OAc)₂ palladium(II) acetate
P(O)Cl₃ phosphorous oxychloride
Rf TLC retention factor
Rt retention time (HPLC)
rt room temperature
MS Mass spectra
THF tetrahydrofuran
TFA trifluoroacetic acid
TLC thin layer chromatography
LC-MS (ESI) liquid chromatography-mass spectroscopy (electrospray ionization)
DIEA diisopropylethylamine
Et₃N triethylamine
MsCl Methanesulfonylchloride
HATU N-[(dimethylamino)-1H-1,2,3-triazolo-[4,5-b]pyridin-1-ylmethylene]-N-methylmethanaminium hexafluorophosphate N-oxide
EDC N-(3-dimethylaminopropyl)-N′-ethylcarbodiimide hydrochloride
AcOH acetic acid
HCl hydrochloric acid
H₂SO₄ sulfuric acid
HNO₃ nitric acid
HBr hydrobromic acid
CDCl₃ chloroform-d
CHCl₃ chloroform
H₂O water
NaOAc sodium acetate
KOH potassium hydroxide
NaOH sodium hydroxide
NaCl sodium chloride
NaHCO₃ sodium bicarbonate
Na₂CO₃ sodium carbonate
K₂CO₃ potassium carbonate
Na₂SO₄ sodium sulfate
MgSO₄ magnesium sulfate
MeOH methanol
SiO₂ silica gel
K₃PO₄ potassium phosphate
NH₄Cl ammonium chloride
AIBN 2,2′-axo bisisobutyronitrile
DMAP N,N-dimethylpyridin-4-amine
PIDA (Diacetoxyiodo)benzene
TsCl p-toluenesulfonyl chloride
PG protecting group
NBS N-Bromosuccinimide
NIS N-Iodosuccinimide
NCS N-Chlorosuccinimide
PE Petroleum ether
FA Formic acid
ee Enantiomeric excess
atm Atmosphere

### Compounds:

The invention provides compounds that modulate, e.g., inhibit, the activity of voltage gated sodium channels. The compounds and their methods of manufacturing are provided in detail below.

### (A) First Set of Compounds

In one aspect, the invention provides a compound of Formula (I):

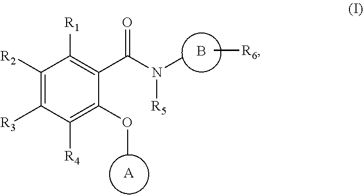

and pharmaceutically acceptable salts, hydrates, and solvates thereof, wherein, A is aryl or heteroaryl wherein the aryl or heteroaryl is unsubstituted or substituted with one or more groups selected from the group consisting of halo-C₁-C₄ alkyl wherein the haloalkyl chain may be fully or partially halogenated, substituted or unsubstituted C₁-C₈ alkyl, deuterated C₁-C₄ alkyl wherein the alkyl chain may be fully or partially deuterated, C₃-C₁₀ cycloalkyl, halogen, cyano, nitro, C₁-C₈ alkoxyl, haloalkoxyl, wherein the haloalkoxy chain may be fully or partially halogenated, or arylalkoxyl;
B is aryl or heteroaryl wherein the aryl may have 1 to 4 substituents and heteroaryl may have 1 to 3 substituents, these substituents are independently selected from halogen, C₁-C₈ alkyl, haloalkyl, or alkoxy;
R₁, R₂, R₃ and R₄ are independently selected from H, halogen, —OH, C₁-C₆-alkyl, C₁-C₆ fluoroalkyl wherein the fluoroalkyl chain may be fully or partially fluorinated, C₃-C₈ branched alkyl, C₃-C₈ branched fluoroalkyl wherein the branched fluoroalkyl chain may be fully or partially fluorinated, alkenyl, alkynyl, haloalkyl, alkoxy, cycloalkoxy, haloalkoxy, thioalkoxy, nitro, cyano, —C(R′)(R″)-cycloalkyl, C(R′)(R″)-aryl, —NR′R″, substituted or unsubstituted 3-8 membered cycloalkyl, 3-8 membered cycloalkenyl, or 3-6 membered heterocycloalkyl, wherein the 3-6 membered heterocycloalkyl comprises at least one heteroatom independently selected from O, S, and N;
R₅ is H or C₁-C₃ alkyl; and
R₆ is —C(═O)NH₂, —C(═O)NHR′, —C(═O)NR′R″, or Formula (II):

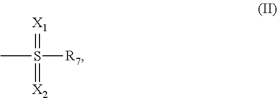

wherein:
X₁ and X₂ are both O, NH, or NR′; or
X₁ is O and X₂ is either NH or NR′;
R₇ is NH₂, NHR′, NR′R″, C₁-C₃ alkyl, C₃-C₈ cycloalkyl, haloalkyl, halocycloalkyl, aryl, heteroaryl, or heterocyclyl.

In various embodiments, the compound of the invention is a compound of Formula (III):

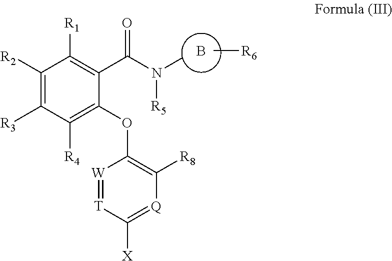

wherein, R₁, R₂, R₃, R₄, R₅, R₆, and B are described in Formula (I);
Q, T and W is independently N or CR₉;
R₉ is H, halogen, —CD₃, alkyl, haloalkyl, alkoxy, haloalkoxy, cyano, —CF₃, —OCF₃, or substituted or unsubstituted cycloalkoxy;
X is H, halogen, —CD₃, alkyl, haloalkyl, alkoxy, cycloalkoxy, haloalkoxy, cyano, —CF₃, — or OCF₃;
R₈ is H, hydroxyl, halogen, —CD₃, C₁-C₆-alkyl, branched alkyl, haloalkyl where the alkyl chain is fully or partially halogenated, alkoxy, arylalkoxy, cycloalkoxy, haloalkoxy, cyano, —CH₂-cycloalkyl, —CH(CH₃)-cycloalkyl, trifluoromethyl, cyclopropylmethyl, or substituted or unsubstituted 3-6 membered cycloalkyl.

In another embodiment, B is

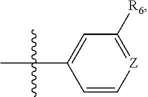

wherein R₆ is described above; Z is CR₁₀, N, or N⁻O⁺; wherein R₁₀ is H, halo, —CD₃, C₁-C₈ alkyl, haloalkyl, or alkoxy.

In another embodiment, R₆ is

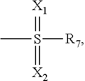

wherein:
X₁ and X₂ are both O, NH, or NR′; or
X₁ is O and X₂ is either NH or NR′;
R₇ is NH₂, NHR′, NR′R″, C₁-C₃ alkyl, C₃-C₈ cycloalkyl, haloalkyl, halocycloalkyl, aryl, heteroaryl, or heterocyclyl.

In another embodiment, R₆ is

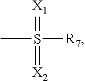

wherein:
X₁ is O, X₂ is NH, and R₇ is C₁-C₃ alkyl.

In another embodiment, R₆ does not include —C(═O)NH₂, —C(═O)NHR′, or —C(═O)NR′R″.

In another embodiment, B is a phenyl ring.

In another embodiment, B is a pyridine ring.

In another embodiment, R₁ is H, —CH₃, or F.

In another embodiment, R₂ is chloro, —CF₃, H, 2-pyrazoline, or 1-methyl-1H-pyrazol-4-yl.

In another embodiment, R₃ is H, —CF₃, or F.

In another embodiment, R₄ is H.

In another embodiment, R₈ is H, —CH₃, or —O—CH₃.

In another embodiment, R₇ is —CH₃.

In another embodiment, X is F or —CN.

In another embodiment, Q is N or CH.

In another embodiment, W is CH and T is CH.

In another embodiment, Z is CR₁₀ and R₁₀ is H or F.

In another embodiment, the compound is selected from a group consisting of:

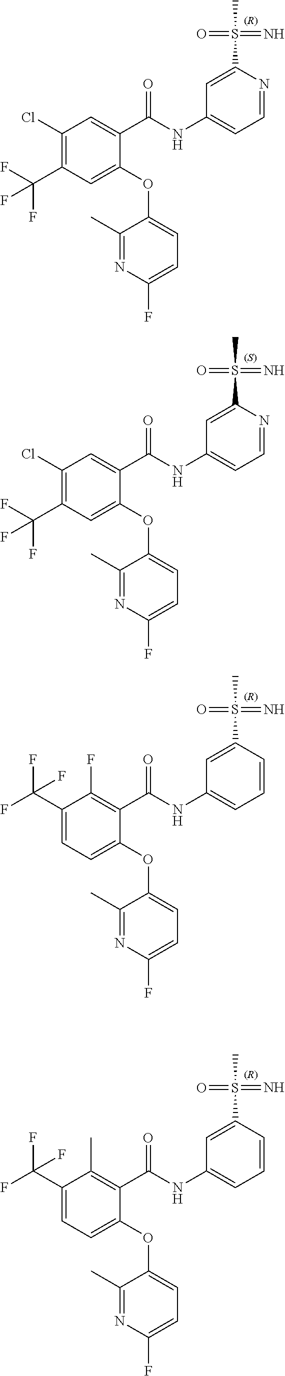

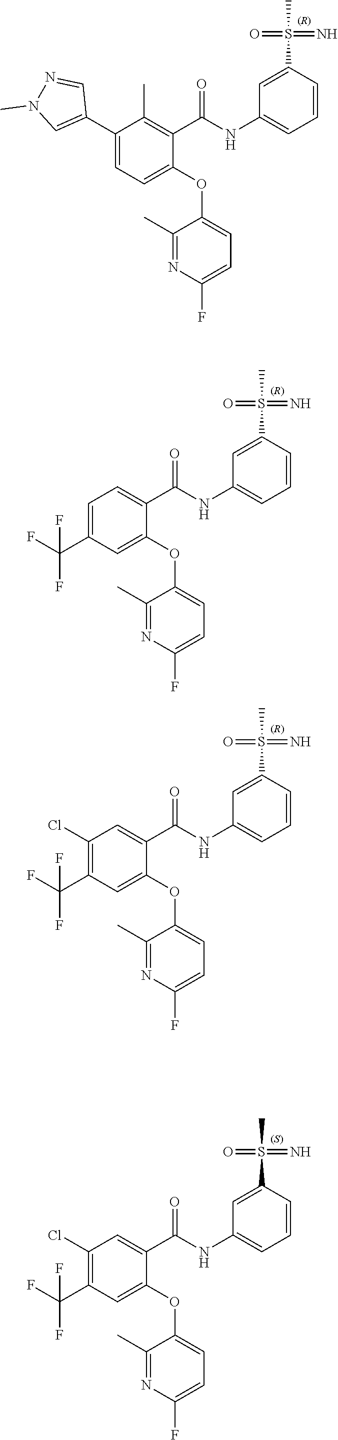

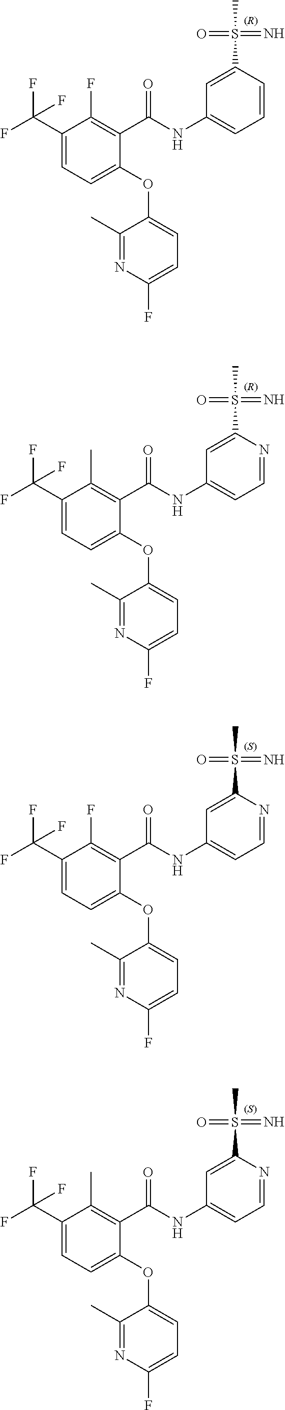

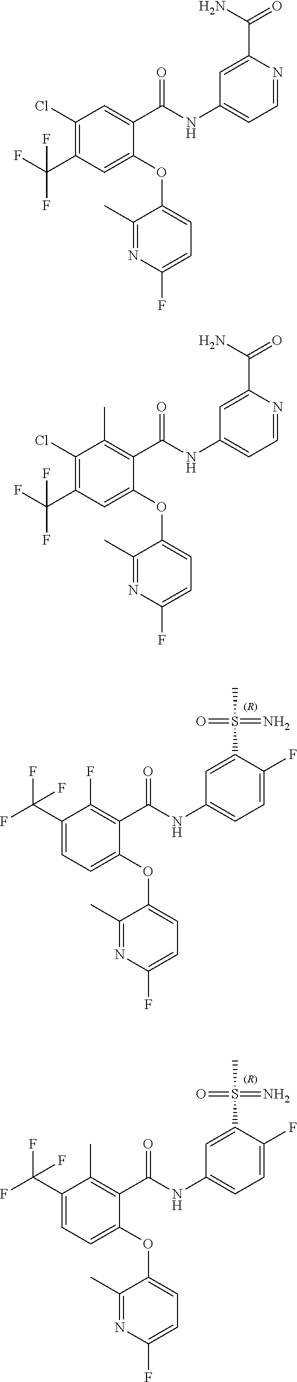

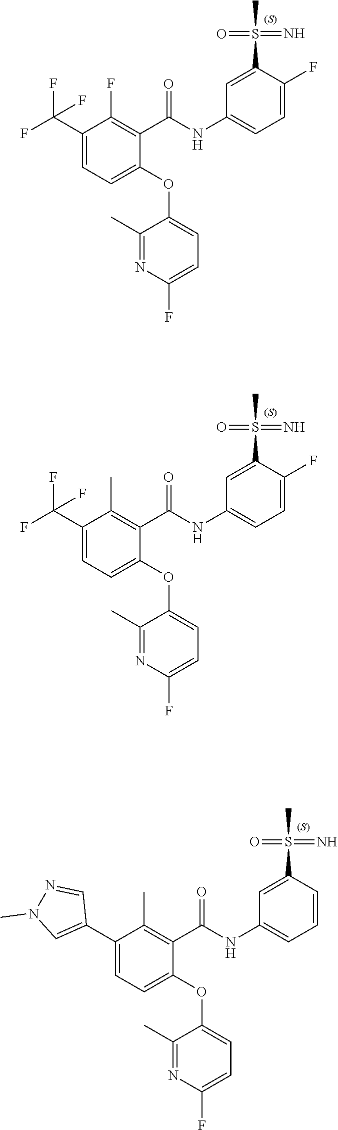

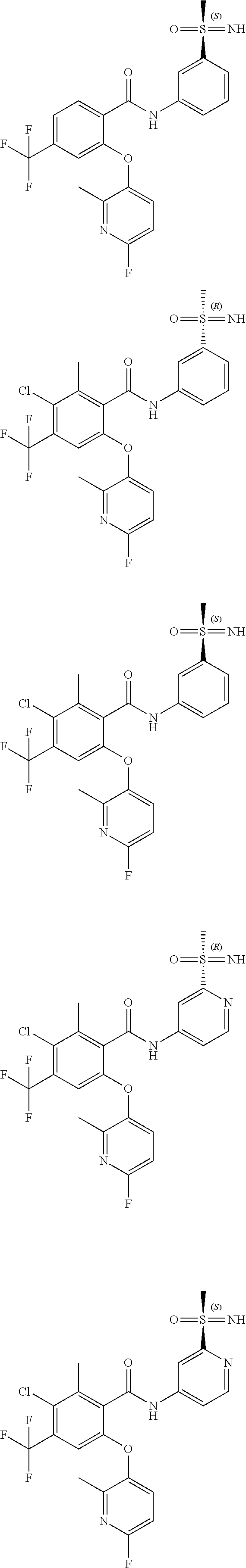

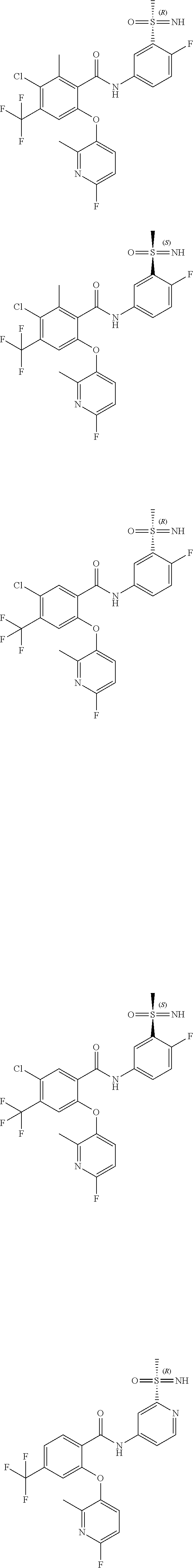

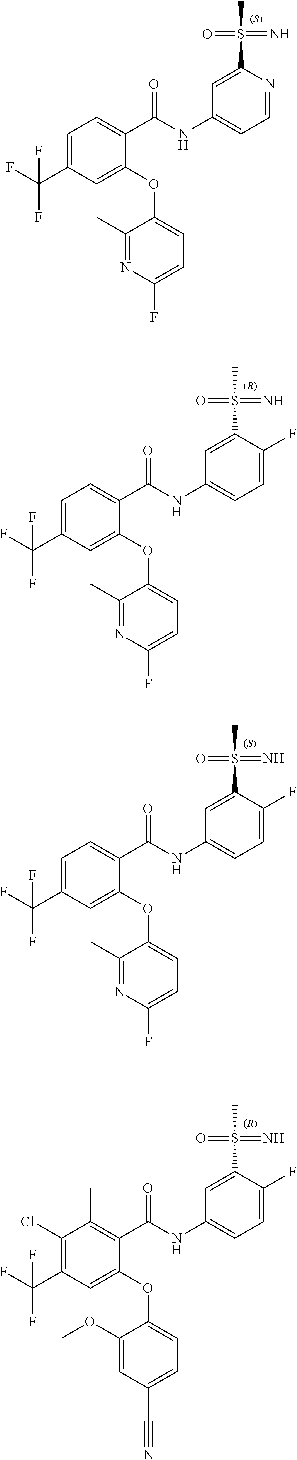

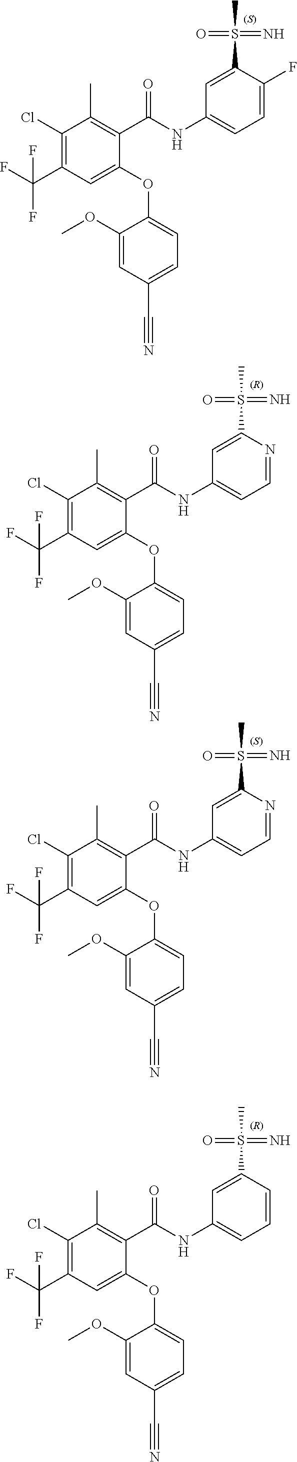

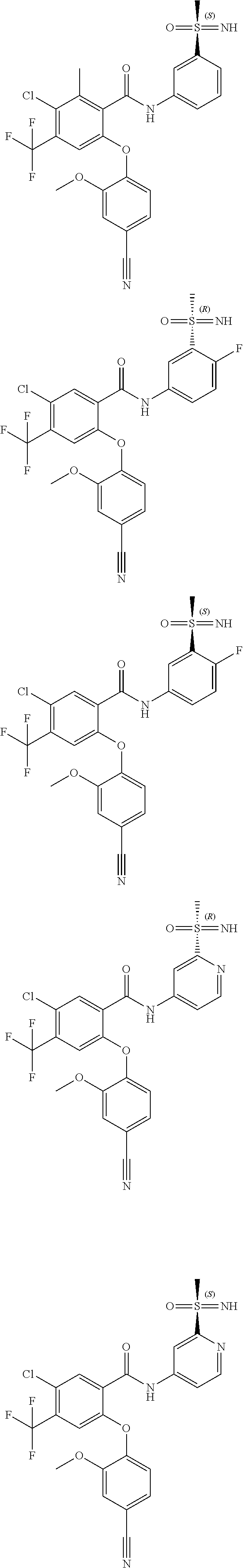

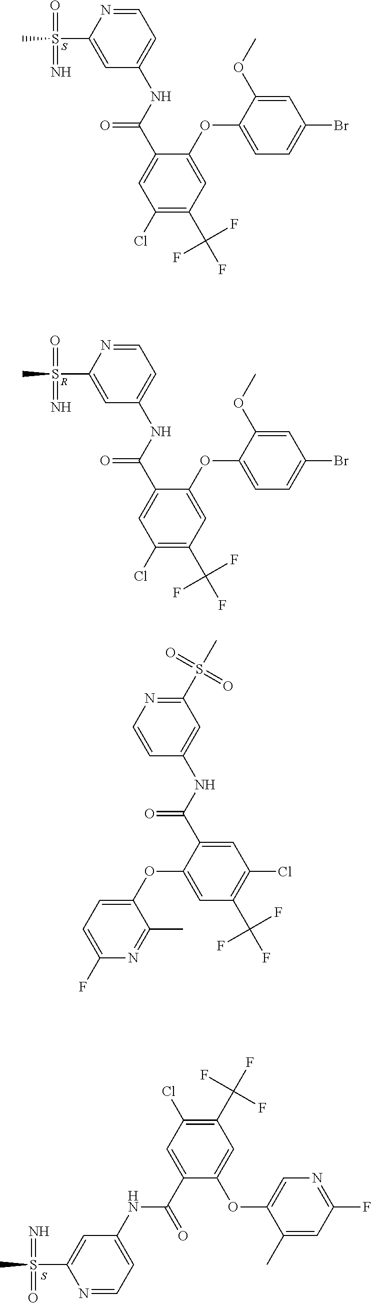

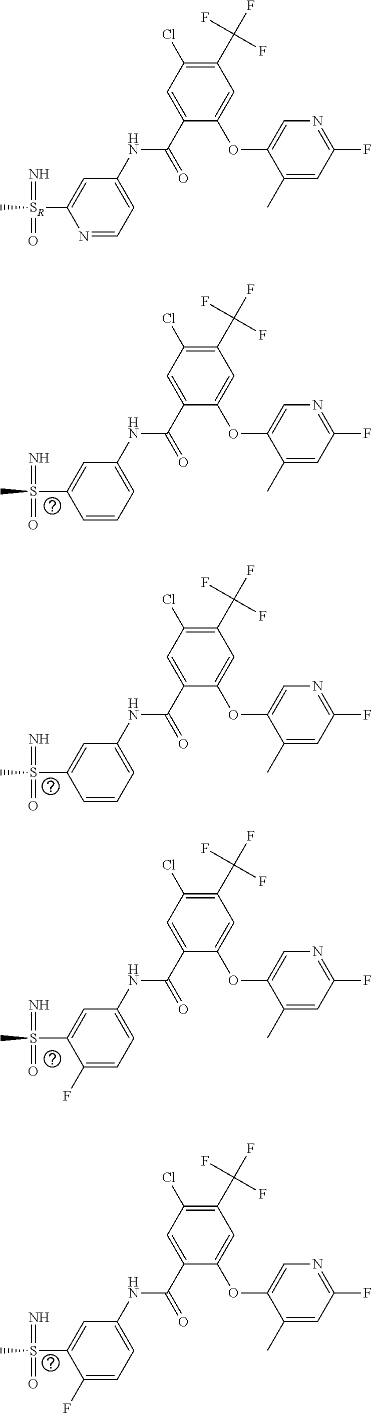

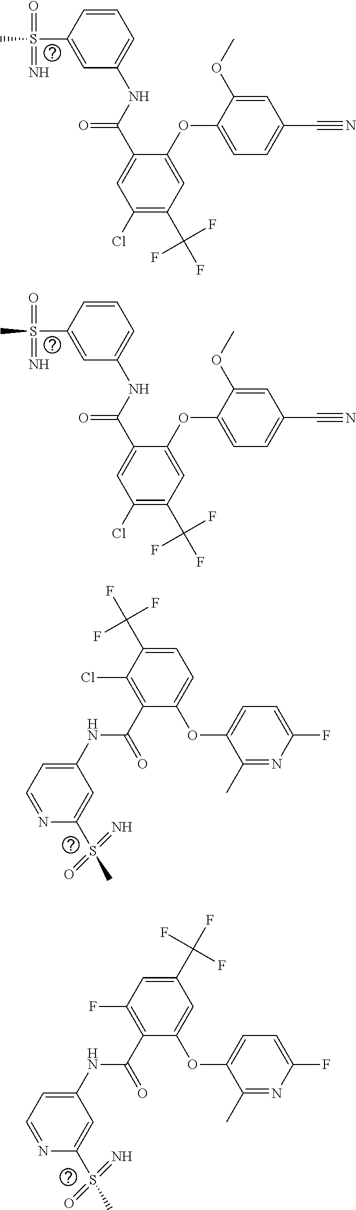 [SMILES: COc1cc(C#N)ccc1Oc1cc(C(F)(F)F)c(Cl)cc1C(=O)Nc1cccc([S@@](C)(=N)=O)c1 ; COc1cc(C#N)ccc1Oc1cc(C(F)(F)F)c(Cl)cc1C(=O)Nc1cccc([S@](C)(=N)=O)c1 ; Cc1nc(F)ccc1Oc1ccc(C(F)(F)F)c(Cl)c1C(=O)Nc1ccnc([S@](C)(=N)=O)c1 ; Cc1nc(F)ccc1Oc1cc(C(F)(F)F)cc(F)c1C(=O)Nc1ccnc([S@@](C)(=N)=O)c1]

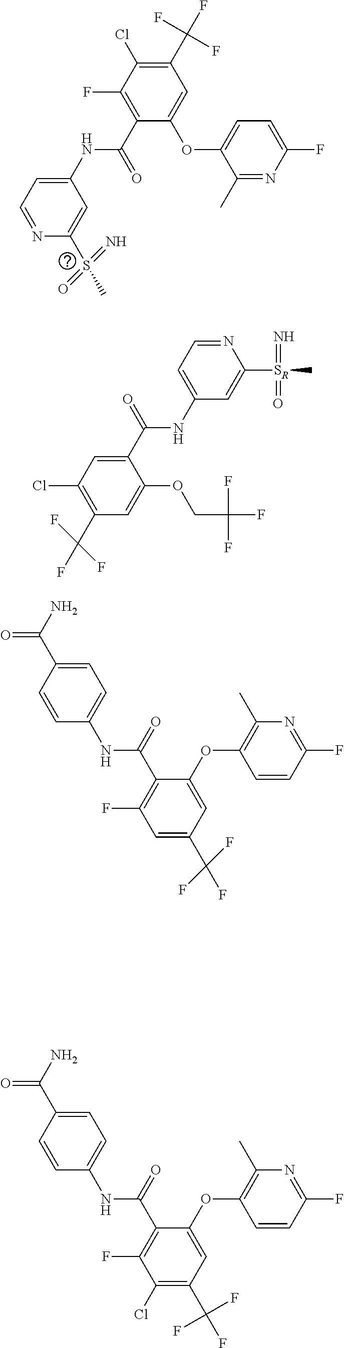

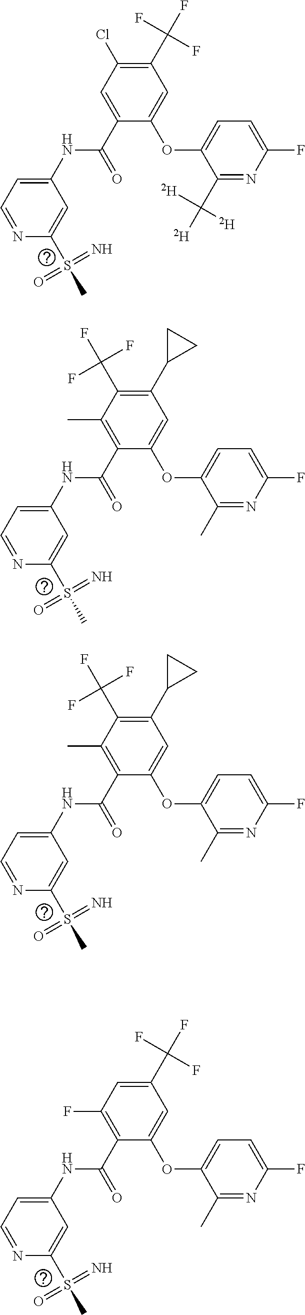

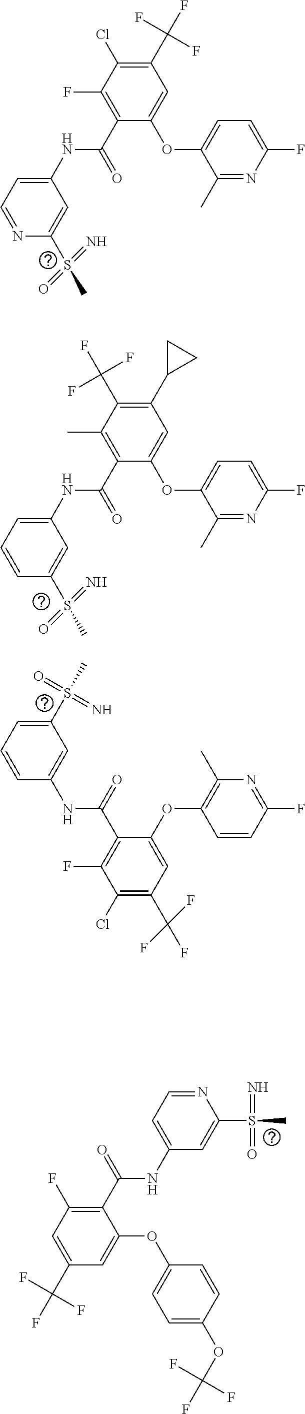

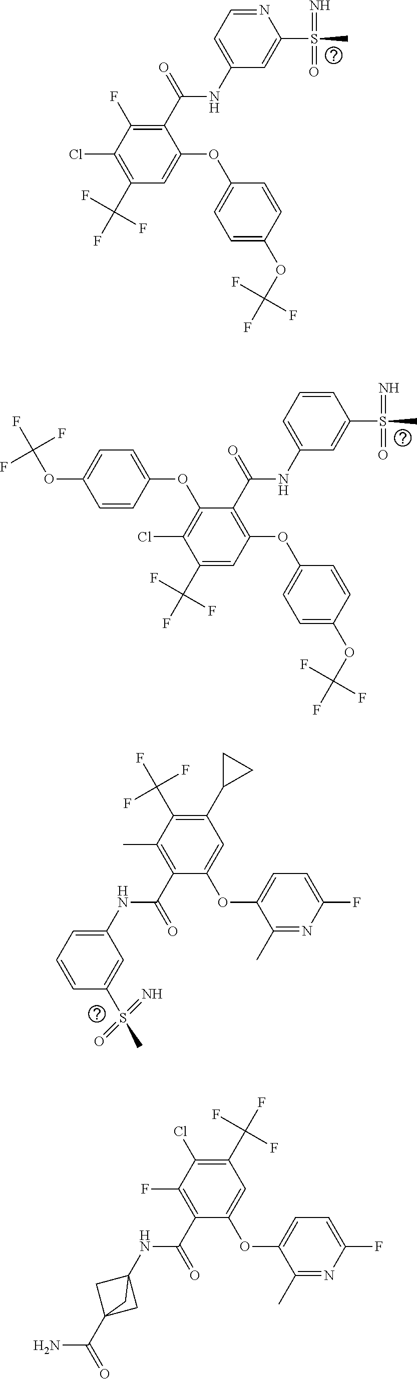 [SMILES: C[S@@](=N)(=O)c1cc(NC(=O)c2c(Oc3ccc(OC(F)(F)F)cc3)cc(C(F)(F)F)c(Cl)c2F)ccn1 ; C[S@@](=N)(=O)c1cccc(NC(=O)c2c(Oc3ccc(OC(F)(F)F)cc3)cc(C(F)(F)F)c(Cl)c2Oc2ccc(OC(F)(F)F)cc2)c1 ; Cc1nc(F)ccc1Oc1cc(C2CC2)c(C(F)(F)F)c(C)c1C(=O)Nc1cccc([S@](C)(=N)=O)c1 ; Cc1nc(F)ccc1Oc1cc(C(F)(F)F)c(Cl)c(F)c1C(=O)NC12CC(C(N)=O)(C1)C2]

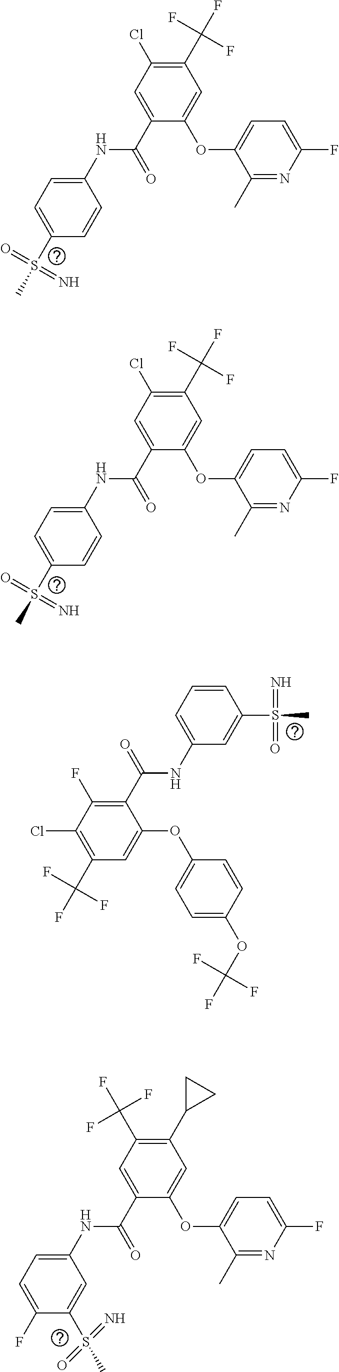 [SMILES: Cc1nc(F)ccc1Oc1cc(C(F)(F)F)c(Cl)cc1C(=O)Nc1ccc([S@@](C)(=N)=O)cc1 ; Cc1nc(F)ccc1Oc1cc(C(F)(F)F)c(Cl)cc1C(=O)Nc1ccc([S@](C)(=N)=O)cc1 ; C[S@@](=N)(=O)c1cccc(NC(=O)c2c(Oc3ccc(OC(F)(F)F)cc3)cc(C(F)(F)F)c(Cl)c2F)c1 ; Cc1nc(F)ccc1Oc1cc(C2CC2)c(C(F)(F)F)cc1C(=O)Nc1ccc(F)c([S@@](C)(=N)=O)c1]

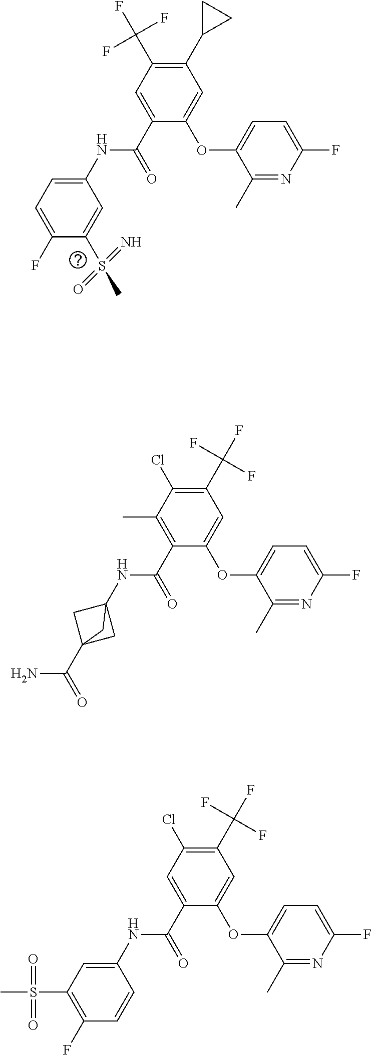 [SMILES: Cc1nc(F)ccc1Oc1cc(C2CC2)c(C(F)(F)F)cc1C(=O)Nc1ccc(F)c([S@](C)(=N)=O)c1 ; Cc1nc(F)ccc1Oc1cc(C(F)(F)F)c(Cl)c(C)c1C(=O)NC12CC(C(N)=O)(C1)C2 ; Cc1nc(F)ccc1Oc1cc(C(F)(F)F)c(Cl)cc1C(=O)Nc1ccc(F)c(S(C)(=O)=O)c1]

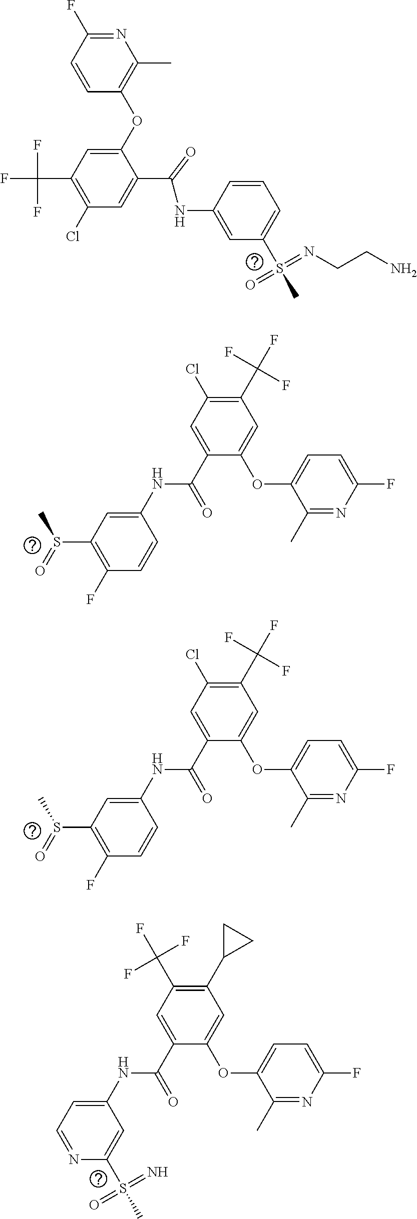 [SMILES: Cc1nc(F)ccc1Oc1cc(C(F)(F)F)c(Cl)cc1C(=O)Nc1cccc([S@@](C)(=O)=NCCN)c1 ; Cc1nc(F)ccc1Oc1cc(C(F)(F)F)c(Cl)cc1C(=O)Nc1ccc(F)c([S@](C)=O)c1 ; Cc1nc(F)ccc1Oc1cc(C(F)(F)F)c(Cl)cc1C(=O)Nc1ccc(F)c([S@@](C)=O)c1 ; Cc1nc(F)ccc1Oc1cc(C2CC2)c(C(F)(F)F)cc1C(=O)Nc1ccnc([S@@](C)(=N)=O)c1]

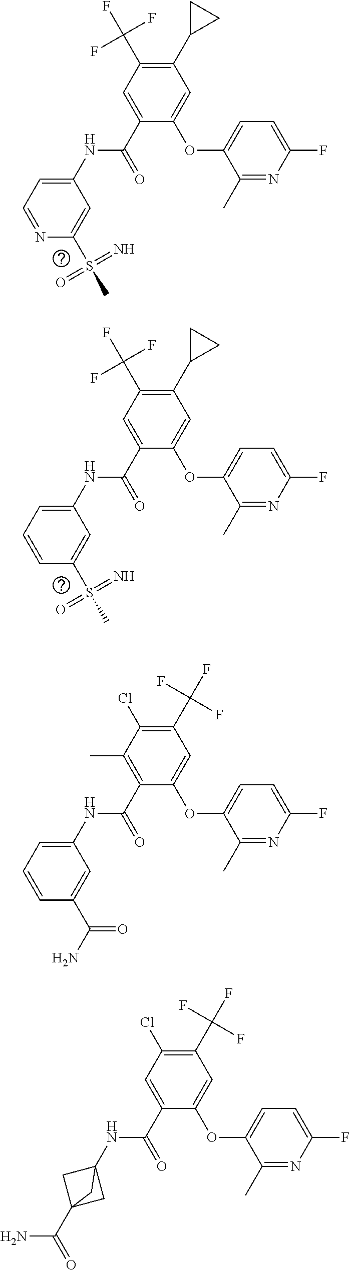

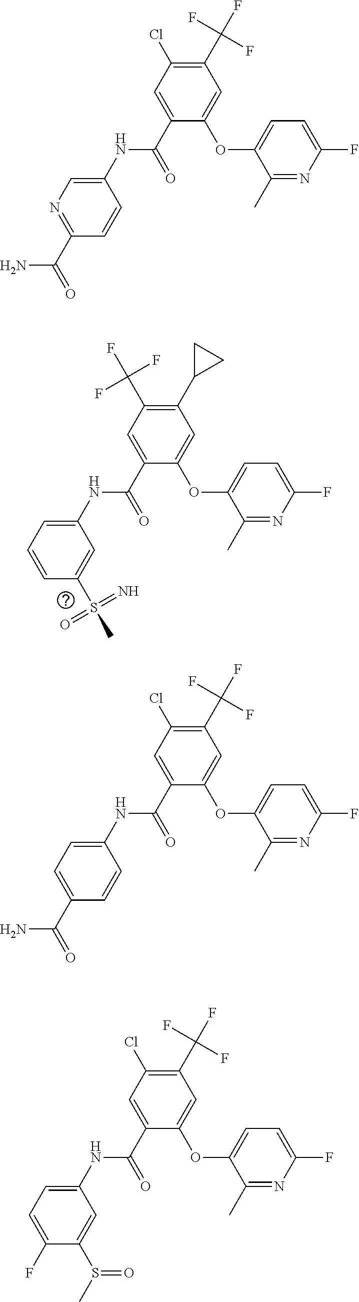 [SMILES: Cc1nc(F)ccc1Oc1cc(C(F)(F)F)c(Cl)cc1C(=O)Nc1ccc(C(N)=O)nc1 ; Cc1nc(F)ccc1Oc1cc(C2CC2)c(C(F)(F)F)cc1C(=O)Nc1cccc([S@](C)(=N)=O)c1 ; Cc1nc(F)ccc1Oc1cc(C(F)(F)F)c(Cl)cc1C(=O)Nc1ccc(C(N)=O)cc1 ; Cc1nc(F)ccc1Oc1cc(C(F)(F)F)c(Cl)cc1C(=O)Nc1ccc(F)c(S(C)=O)c1]

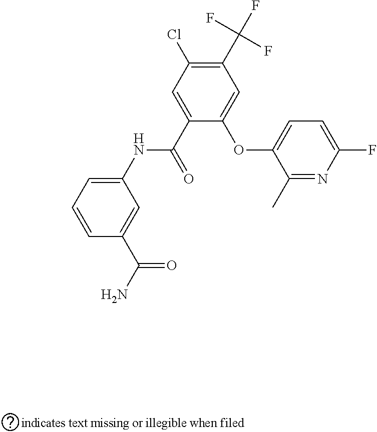 [SMILES: Cc1nc(F)ccc1Oc1cc(C(F)(F)F)c(Cl)cc1C(=O)Nc1cccc(C(N)=O)c1]

The first set of compounds, provided herein, are prepared by methods and procedures described below. The intermediates described in this section may be relied upon for preparation of first set of compounds.

Chemicals were purchased from standard commercial vendors and used as received unless otherwise noted. Otherwise, their preparation is facile and known to one of ordinary skill in the art, or it is referenced or described herein. Abbreviations are consistent with those in the ACS Style Guide. “dry” glassware means oven/desiccator dried. Solvents were ACS grade unless otherwise noted.

All reactions were performed in flame-dried or oven-dried glassware under a positive pressure of dry nitrogen or dry argon and were stirred magnetically unless otherwise indicated. Chemicals were purchased from standard commercial vendors and used as received unless otherwise noted. Yields are not optimized. The chemical names were generated using the ChemDraw Professional 19.1, available from PerkinElmer or chemAxon.

Reactions were monitored by thin layer chromatography (TLC) using 0.25 mm silica gel 60 F254 plates purchased from EMD MILLIPORE™. Purification was performed with Biotage Isolera One Flash Chromatography Instrument or purified using one of the preparative HPLC methods mentioned below.

### Prep Method 1

Equipment: Shimadzu LCMS 2020 mass-directed preparative HPLC System; column: Gemini 5 um C₁₈ column, 150*21.2 mm; General gradient: 30% to 90% MeCN/H₂O containing 0.1% HCOOH, gradient may be slight adjusted for specific compound; Flow rate: 20 mL/min; Column temperature: ambient temperature; UV Wavelength: 214 and 254 nm;

### Prep Method 2

Equipment: Shimadzu LC-20AP Preparative HPLC System; column: Gemini 5 um C₁₈ column, 150*21.2 mm; General gradient: 30% to 90% MeCN/H₂O containing 0.1% TFA, gradient may be slight adjusted for specific compound; Flow rate: 20 mL/min; Column temperature: ambient temperature; UV Wavelength: 214 and 254 nm.

### Prep Method 3

Equipment: Shimadzu LC-20AP Preparative HPLC System; column: Gemini 5 um C₁₈ column, 150×21.2 mm; General gradient: 30% to 90% MeCN/H₂O containing 0.05% ammonia, gradient may be slight adjusted for specific compound; Flow rate: 20 mL/min; Column temperature: ambient temperature; UV Wavelength: 214 and 254 nm.

Analytical LCMC were collected using one of following methods—

### Method 1

Equipment: Shimadzu LCMS 2020 Mass Spectrometer; Column: HALO C₁₈ 2.7 μm, 3.0 mm×30 mm; Mobile Phase: MeCN (0.05% HCOOH)—Water (0.05% HCOOH); Gradient: MeCN from 5% to 95% over 1.4 min, hold 0.6 min, total run time is 2.5 min; Flow rate: 1.8 mL/min; Column temperature: 50° C.; Wavelength: 214 and 254 nm PDA.

### Method 2

Equipment: Shimadzu LCMS 2020 Mass Spectrometer; XBridge BEH C₁₈ 2.5 μm, 3.0 mm×30 mm Mobile Phase: MeCN—Water (0.1% NH₄OH); Gradient: MeCN from 5% to 95% over 1.8 min, hold 0.7 min, total run time is 3.0 min; Flow rate: 1.0 mL/min; Column temperature: 50° C.; Wavelength: 214 and 254 nm PDA.

### Method 3

Equipment: Shimadzu LCMS 2020 Mass Spectrometer; Column: HALO C₁₈ 2.7 μm, 3.0 mm×30 mm Mobile Phase: MeCN (0.05% TFA)—Water (0.05% TFA); Gradient: MeCN from 5% to 95% over 1.4 min, hold 0.6 min, total run time is 2.5 min; Flow rate: 1.8 mL/min; Column temperature: 50° C.; Wavelength: 214 and 254 nm PDA.

SFC chiral resolution was performed on Shimadzu Nexera UC Preparative SFC System (SFE-30A, LC-30AD_{SF}, SFC-30A) using following methods:

### Method 1

Column: Daicel chiralpak-AS-H 5 μm 250×20 mm; Mobile Phase: CO₂/MeOH [0.1% NH₃ (7M in MeOH)], CO₂/MeOH ratio varies for different compounds; Oven temperature: 40° C.; Flow rate: 38 mL/min.

### Method 2

Column: Daicel chiralpak-OJ-H 5 μm 250*20 mm; Mobile Phase: CO₂/MeOH (0.1% HCOOH), CO₂/MeOH ratio varies for different compounds; Oven temperature: 40° C.; Flow rate: 38 mL/min.

### Method 3

Column: Daicel chiralpak-OD-H 5 μm 250×20 mm; Mobile Phase: CO₂/MeOH, ratio varies for different compounds; Oven temperature: 40° C.; Flow rate: 38 mL/min.

### Method 4

Column: Daicel chiralpak-AD-H 5 μm 250×20 mm; Mobile Phase: CO₂/i-PrOH, ratio varies for different compounds; Oven temperature: 40° C.; Flow rate: 38 mL/min.

### Method 5

Column: Daicel chiralpak-IC 5 μm 250×20 mm; Mobile Phase: CO₂/EtOH, ratio varies for different compounds; Oven temperature: 40° C.; Flow rate: 38 mL/min.

Unless otherwise stated, ¹H nuclear magnetic resonance spectroscopy (NMR) spectra were recorded on a Bruker AVANCE NEO 400 MHz Digital NMR Spectrometer. Chemical shifts, 6, are quoted in parts per million (ppm) relative to TMS and calibrated using residual un-deuterated solvent as an internal reference. The following abbreviations are used to denote the multiplicities and general assignments: s (singlet), d (doublet), t (triplet), q (quartet), dd (doublet of doublets), ddd (doublet of doublet of doublets), dt (doublet of triplets), dq (doublet of quartets), hep (heptet), m (multiplet), pent (pentet), td (triplet of doublets), qd (quartet of doublets), app. (apparent) and br. (broad). Coupling constants, J, are quoted to the nearest 0.1 Hz.

### General Synthetic Schemes

Several methods for preparing the compounds of this invention are illustrated in the following Schemes and Examples. The present invention further provides processes for the preparation of compounds of structural Formula I as defined above. In some cases, the order of carrying out the foregoing reaction schemes may be varied to facilitate the reaction or to avoid unwanted reaction products. The following examples are provided for the purpose of illustration only and are not to be construed as limitations on the disclosed invention.

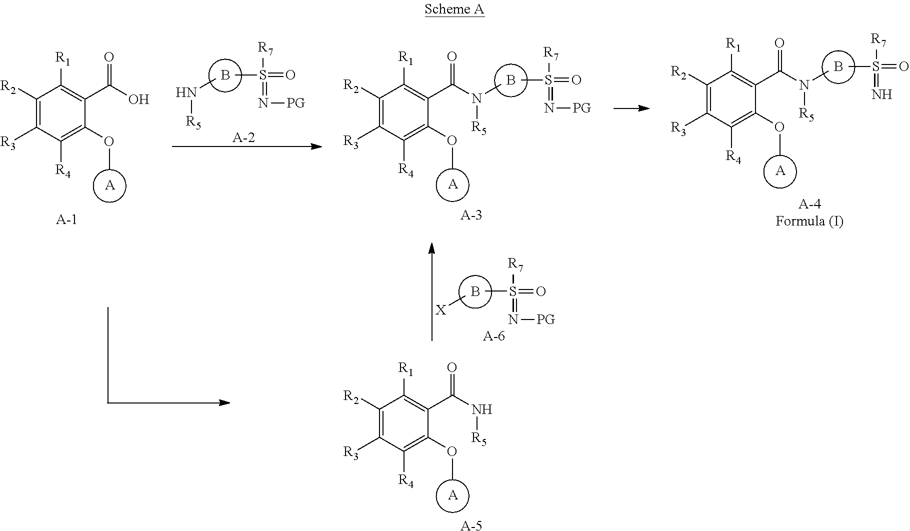

As illustrated in Scheme A, in general, compounds of Formula (I) may be synthesized starting from carboxylic acids A-1 by reacting with a substituted aniline or heteroaryl aniline A-2 using standard amide coupling reagents, not limited to HATU, TBTU, EDC or T3P in organic solvents and base, such as DIEA, to give intermediates of type A-3. Intermediates A-3 may contain a protecting group (PG) such as Boc which may be removed by treatment with an acid such as TFA to provide compounds of Formula (I) having structure A-4. Any suitable PG such as Cbz or Fmoc may be employed and can be removed accordingly. Alternatively, carboxylic acid A-1 may be treated with ammonia or a primary amine (R₅NH₂) in the presence of an amide coupling reagent such as HATU and a base such as DIEA to give carboxamide intermediate A-5 which can undergo metal-catalyzed coupling with a halogen-substituted aryl or heteroaryl compounds of type A-6 to give compounds of formula A-3. Compounds of Formula (I) can then be obtained from A-3 as described.

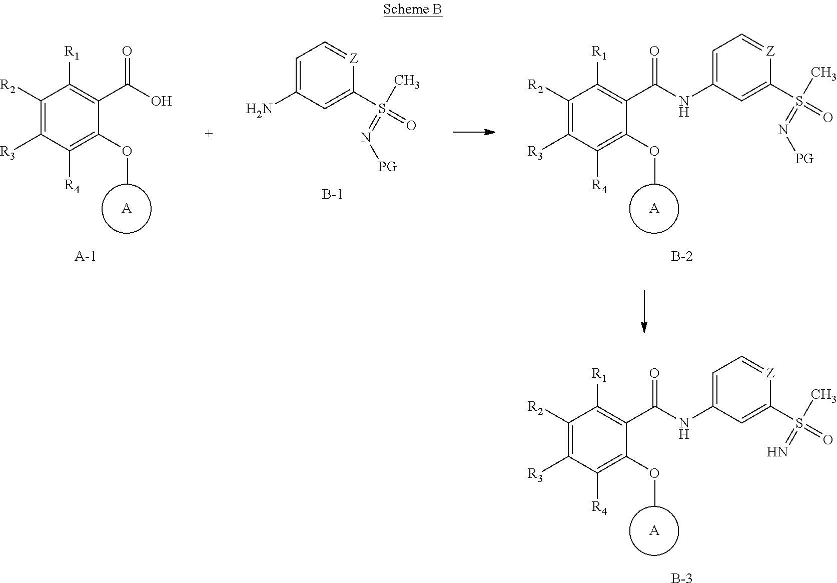

As further illustrated in Scheme B, in general, compounds of the invention can be prepared by reacting intermediates A-1 with an amine B-1, utilizing amide coupling conditions or by activation of an appropriately functionalized carboxylic acid A-1 with (COCl)₂ or POCl₃ and with amine B-1 and base such as DIEA or pyridine in DCM, DMF or THF to give compounds of type B-2. The compounds of formula B-3 can be formed by removing a protecting group, such as Boc under acidic conditions. In some instances, B-3 can be separated into the corresponding R and S isomers using chiral HPLC. R- and S-isomers also can be prepared by coupling of the acid to enantiomerically pure amine B-1 followed by deprotection.

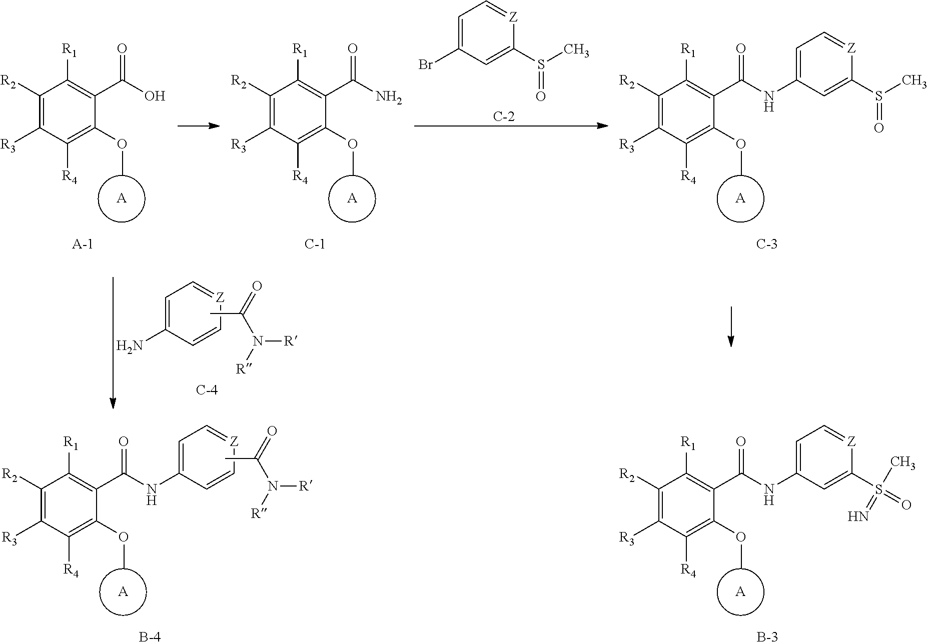

As illustrated in Scheme C, in general, compounds of the invention can be prepared by activation of appropriately functionalized carboxylic acid A-1 in organic solvent with either (COCl)₂ or SOCl₂ followed by addition of NH₄OH to afford C-1. Intermediate C-1 can then be brought together with materials of variously substituted Br compounds C-2, utilizing Xantphos-Pd-G2 mediated coupling conditions to deliver intermediate C-3. The compounds of formula C-3 may be treated with ammonium carbonate or ammonium carbamate and (diacetoxyiodo)benzene (PIDA) in methanol to provide compounds of formula B-3. In some instances, B-3 was separated to the corresponding R and S isomers using chiral HPLC conditions. Compounds of formula B-4 were obtained using standard amide coupling reagents such as, not limited to, HATU, a base such as DIEA, and C-4.

The above schemes are intended to be illustrative and not limiting in any way. Those skilled in the art may prepare the Compounds of Formula (I) using these general schemes as a guideline but other methods are available to accomplish the synthesis of compounds of the invention. Specific methods are provided for each of the Examples to further illustrate the synthesis of Compounds of Formula (I).

### Intermediates

### Intermediate 1

### 5-chloro-2-((6-fluoro-2-methylpyridin-3-yl)oxy)-4-(trifluoromethyl)benzoic acid

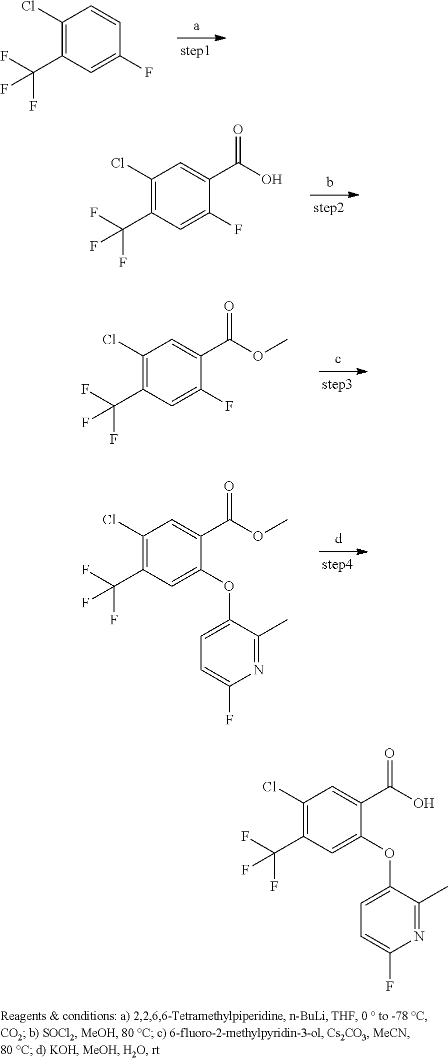 [SMILES: Fc1ccc(Cl)c(C(F)(F)F)c1 ; O=C(O)c1cc(Cl)c(C(F)(F)F)cc1F ; COC(=O)c1cc(Cl)c(C(F)(F)F)cc1F ; COC(=O)c1cc(Cl)c(C(F)(F)F)cc1Oc1ccc(F)nc1C ; Cc1nc(F)ccc1Oc1cc(C(F)(F)F)c(Cl)cc1C(=O)O]

Step 1: 5-chloro-2-fluoro-4-(trifluoromethyl)benzoic acid: To a solution of Me₄-piperidine (3.2 g, 23 mmol) in THF (30 mL) was added n-BuLi (2.4 M in hexane, 9.58 mL, 23 mmol) dropwise at 0° C. under an atmosphere of N₂. The mixture was stirred at 0° C. for 1 hour. Then a solution of 1-chloro-4-fluoro-2-(trifluoromethyl)benzene (3 g, 15 mmol) in THF (30 mL) was added dropwise to the mixture at −78° C. The mixture was stirred at −78° C. for 2 hours. The resulting solution was added dry ice (about 5 g). Then the reaction mixture was slowly warmed to 0° C. and stirred for 1 hour at the same temperature. The final mixture was quenched with saturated aqueous NH₄Cl (100 mL) and extracted with EtOAc (100 mL×3). The combined organic layers were washed with brine, dried over Na₂SO₄, concentrated under vacuum. The residue was purified by flash column chromatography on silica gel (DCM/MeOH=20/1) to give 5-chloro-2-fluoro-4-(trifluoromethyl)benzoic acid (2 g, 54.6%) as a yellow solid. LCMS (ESI) calcd. for C₈H₂ClF₄O₂ [M−H]⁻ m/z 240.97, found 240.85.

Step 2: methyl 5-chloro-2-fluoro-4-(trifluoromethyl)benzoate: A solution of 5-chloro-2-fluoro-4-(trifluoromethyl)benzoic acid (2 g, 8.30 mmol) in SOCl₂ (20 mL) was heated at 80° C. for 1 h. Then the mixture was concentrated in vacuum. The residue was added to MeOH (30 mL) and stirred at room temperature for 1 hour. The final mixture was concentrated under vacuum to give the crude methyl 5-chloro-2-fluoro-4-(trifluoromethyl)benzoate (1.8 g) which was used directly in next step without further purification.

Step 3: methyl 5-chloro-2-((6-fluoro-2-methylpyridin-3-yl)oxy)-4-(trifluoromethyl)benzoate: A solution of methyl 5-chloro-2-fluoro-4-(trifluoromethyl)benzoate (1.8 g, 7.03 mmol), 6-fluoro-2-methylpyridin-3-ol (982 mg, 7.73 mmol), and Cs₂CO₃ (4.6 g, 14.06 mmol) in MeCN (40 mL) was heated at 80° C. for 16 hours. Upon completion of the reaction, the mixture was concentrated under vacuum. The residue was directly purified by flash column chromatography on silica gel (PE/EtAOc=3/1) to give methyl 5-chloro-2-((6-fluoro-2-methylpyridin-3-yl)oxy)-4-(trifluoromethyl)benzoate (2.4 g, 93.93%) as a yellow solid. LCMS (ESI) calcd. for C₁₅H₁₁ClF₄NO₃ [M+H]⁻ m/z 364.04, found 363.70.

Step 4: 5-chloro-2-((6-fluoro-2-methylpyridin-3-yl)oxy)-4-(trifluoromethyl)benzoic acid: To a solution of methyl 5-chloro-2-((6-fluoro-2-methylpyridin-3-yl)oxy)-4-(trifluoromethyl)benzoate (1 g, 2.76 mmol) in MeOH/H₂O (1/1, 30 mL) was added KOH (907 mg 13.77 mmol). The mixture was stirred at room temperature for 2 hours. Then the organic solvent was removed under reduced pressure. The aqueous solution was adjusted to pH=2-3 with 2N HCl. The precipitate was collected by filtration, washed with water and dried under vacuum to provide 5-chloro-2-((6-fluoro-2-methylpyridin-3-yl)oxy)-4-(trifluoromethyl)benzoic acid (900 mg, 93.65%) as a yellow solid. LCMS (ESI) calcd. for C₁₄H₉ClF₄NO₃ [M+H]⁺ m/z 350.02, found 349.65.

### Intermediate 2

### 2-((6-fluoro-2-methylpyridin-3-yl)oxy)-4-(trifluoromethyl)benzoic acid

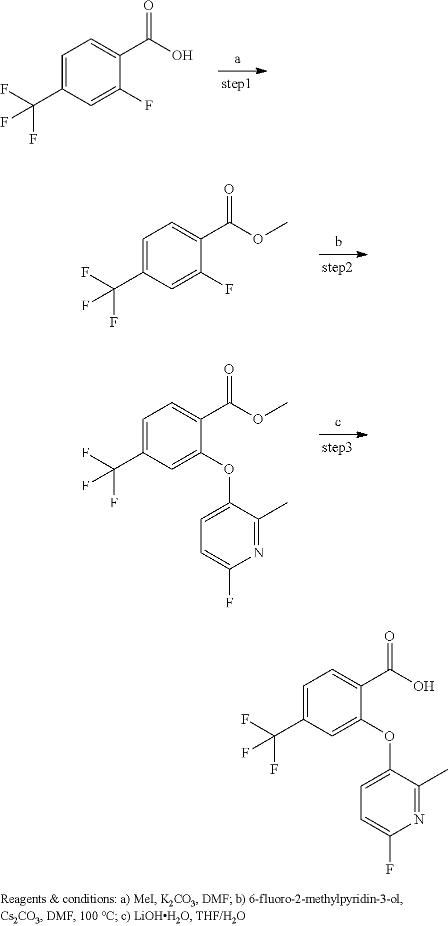 [SMILES: O=C(O)c1ccc(C(F)(F)F)cc1F ; COC(=O)c1ccc(C(F)(F)F)cc1F ; COC(=O)c1ccc(C(F)(F)F)cc1Oc1ccc(F)nc1C ; Cc1nc(F)ccc1Oc1cc(C(F)(F)F)ccc1C(=O)O]

Step 1: methyl 2-fluoro-4-(trifluoromethyl)benzoate: To a solution of 2-fluoro-4-(trifluoromethyl)benzoic acid (500 mg, 2.4 mmol) and K₂CO₃ (993 mg, 7.2 mmol) in DMF (5 mL) CH₃I (677 mg, 4.8 mmol) was added. The reaction mixture was stirred at room temperature for 18 hours. After the reaction was completed, the resulting solution was diluted with water (50 mL) and extracted with EtOAc (50 mL×3). The combined organic layers were washed with brine, dried over sodium sulfate, concentrated under vacuum. The residue was purified by flash column chromatography on silica gel (PE/EtOAc=10/1 to 5/1) to give methyl 2-fluoro-4-(trifluoromethyl)benzoate (490 mg, 81.7%) as a white oil. LCMS (ESI) calcd. for C₉H₇F₄O₂ [M+H]⁺ m/z 223.04, found 223.04.

Step 2: methyl 2-((6-fluoro-2-methylpyridin-3-yl)oxy)-4-(trifluoromethyl)benzoate: A solution of methyl 2-fluoro-4-(trifluoromethyl)benzoate (400 mg, 1.8 mmol) in DMF (5 mL) Cs₂CO₃ (1.75 g, 5.4 mmol), 6-fluoro-2-methylpyridin-3-ol (342 mg, 2.7 mmol) was added. The mixture was heated to 100° C. for 1 hour. After the reaction was completed, the mixture was filtered through celite. The solution was concentrated under vacuum and purified by flash column chromatography on silica gel (PE/EtOAc=4/1 to 3/1) to provide methyl 2-((6-fluoro-2-methylpyridin-3-yl)oxy)-4-(trifluoromethyl)benzoate (350 mg, 86%) as a light-yellow solid. LCMS (ESI) calcd. for C₁₅H₁₂F₄NO₃ [M+H]⁺ m/z 330.08, found 330.00.

Step 3: 2-((6-fluoro-2-methylpyridin-3-yl)oxy)-4-(trifluoromethyl)benzoic acid: To a solution of methyl 2-((6-fluoro-2-methylpyridin-3-yl)oxy)-4-(trifluoromethyl)benzoate (350 mg, 1.06 mmol) in THF (4 mL) and H₂O (4 mL) was added LiOH·H₂O (267 mg, 6.36 mmol) at room temperature. The reaction mixture was stirred at room temperature for 18 hours. After the reaction was completed, the residue was adjusted to pH=3-4 with HCl (1 M). Then the aqueous solution was extracted with EtOAc (20 mL×3). The combined organic layers were washed with brine, dried over sodium sulfate, concentrated under vacuum to provide 2-((6-fluoro-2-methylpyridin-3-yl)oxy)-4-(trifluoromethyl)benzoic acid (300 mg, 92%) as a white solid. LCMS (ESI) calcd. for C₁₄H₁₀F₄NO₃ [M+H]⁺ m/z 316.06, found 315.90.

### Intermediate 3

### 6-((6-fluoro-2-methylpyridin-3-yl)oxy)-2-methyl-3-(1-methyl-1H-pyrazol-4-yl)benzoic acid

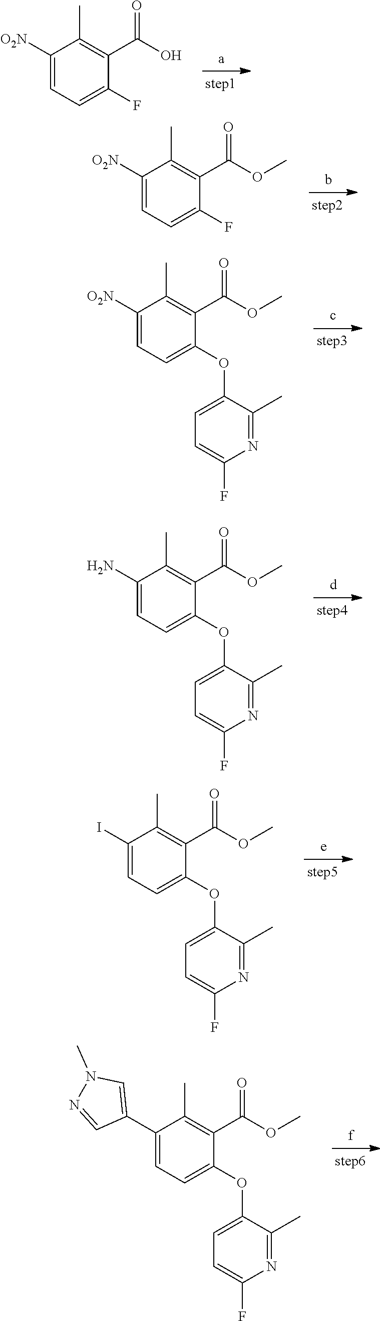

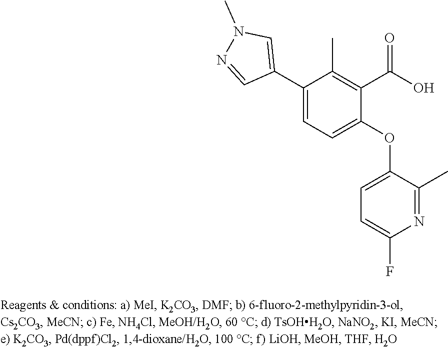 [SMILES: Cc1nc(F)ccc1Oc1ccc(-c2cnn(C)c2)c(C)c1C(=O)O]

Step 1: methyl 6-fluoro-2-methyl-3-nitrobenzoate: To a solution of 6-fluoro-2-methyl-3-nitrobenzoic acid (2.00 g, 10.04 mmol) in DMF (20 mL) was added K₂CO₃ (2.78 g, 20.09 mmol) and MeI (2.14 g, 15.07 mmol). The mixture was stirred at room temperature for 12 hours. After the reaction was completed, solution was diluted with DCM (100 mL) and washed with brine (100 mL×3), dried over sodium sulfate, then concentrated under vacuum. The residue was purified by flash column chromatography on silica gel (PE/EtOAc=1/1 to 1/3) to provide methyl 6-fluoro-2-methyl-3-nitrobenzoate (2.00 g, 93.4%) as a white solid.

Step 2 methyl 6-((6-fluoro-2-methylpyridin-3-yl)oxy)-2-methyl-3-nitrobenzoate: A mixture of methyl 6-fluoro-2-methyl-3-nitrobenzoate (1.70 g, 7.98 mmol) and 6-fluoro-2-methylpyridin-3-ol (1.22 g, 9.57 mmol) in MeCN (30 mL) was added Cs₂CO₃ (3.31 g, 23.93 mmol) at room temperature. The reaction mixture was heated at 80° C. for 16 hours. After the reaction was completed, the solution was diluting with DCM (150 mL) and washed with brine (150 mL), dried over sodium sulfate, then concentrated and purified by flash column chromatography on silica gel (Pe/EtOAc=5/1 to 3/1) to provide methyl 6-((6-fluoro-2-methylpyridin-3-yl)oxy)-2-methyl-3-nitrobenzoate (1.80 g, 70.5%) as a white solid. LCMS (ESI) calcd. for C₁₅H₁₄FN₂O₅ [M+H]⁺ m/z 321.09, found 321.05.

Step 3: methyl 3-amino-6-((6-fluoro-2-methylpyridin-3-yl)oxy)-2-methylbenzoate: A solution of methyl 6-((6-fluoro-2-methylpyridin-3-yl)oxy)-2-methyl-3-nitrobenzoate (1.70 g, 5.31 mmol) in MeOH (30 mL) and water (10 mL) was added NH₄Cl (1.99 g, 37.16 mmol), Fe (1.48 g, 26.54 mmol). The mixture was heated to 60° C. for 1 hour. After the reaction was completed, the mixture was filtered through celite. The filtrate was diluted with water (100 mL) and extracted with EtOAc (50 mL×3). The combined organic layers were washed with brine, dried over sodium sulfate, concentrated under vacuum to provide methyl 3-amino-6-((6-fluoro-2-methylpyridin-3-yl)oxy)-2-methylbenzoate (1.50 g, 97.3%) as a red oil. LCMS (ESI) calcd. for C₁₅H₁₆FN₂O₃ [M+H]⁺ m/z 291.12, found 291.05.

Step 4: methyl 6-((6-fluoro-2-methylpyridin-3-yl)oxy)-3-iodo-2-methylbenzoate: To a solution of TsOH-H₂O (2.95 g, 15.50 mmol) and methyl 3-amino-6-((6-fluoro-2-methylpyridin-3-yl)oxy)-2-methylbenzoate (1.50 g, 5.17 mmol) in MeCN (30 mL) was added a solution of NaNO₂ (713 mg, 10.33 mmol) and KI (2.14 g, 12.92 mmol) in H₂O (5 mL) at 0° C. The mixture was stirred at room temperature for 2.5 hours. After the reaction was completed, the mixture was quenched with water (100 mL), adjusted to pH=8-9 with saturated aqueous NaHCO₃. The solution was extracted with EtOAc (200 mL×3). The combined organic layers were washed with brine, dried over sodium sulfate, concentrated under vacuum. The residue was purified by flash column chromatography on silica gel (PE/EtOAc=10/1 to 1/1) to provide methyl 6-((6-fluoro-2-methylpyridin-3-yl)oxy)-3-iodo-2-methylbenzoate (1.50 g, 72.4%) as yellow solid. LCMS (ESI) calcd. for C₁₅H₁₄FINO₃ [M+H]⁺ m/z 402.00, found 401.90.

Step 5: methyl 6-((6-fluoro-2-methylpyridin-3-yl)oxy)-2-methyl-3-(1-methyl-1H-pyrazol-4-yl)benzoate: A mixture of methyl 6-((6-fluoro-2-methylpyridin-3-yl)oxy)-3-iodo-2-methylbenzoate (400 mg, 1.00 mmol) and (1-methyl-1H-pyrazol-4-yl)boronic acid (188 mg, 1.50 mmol) in 1,4-dioxane/H2O (4/1, 10 mL) was added potassium carbonate (413 mg, 2.99 mmol) and Pd(dppf)Cl₂ (146 mg, 0.20 mmol). The mixture was heated at 100° C. for 6 hours under an atmosphere of N₂. The reaction was monitored by LCMS. After the reaction was completed, the mixture was cooled to room temperature. The resulting solution was diluted with water (20 mL) and extracted with DCM (20 mL×3). The combine organic layers were washed with brine, dried over Na₂SO₄, concentrated under vacuum. The residue was purified by flash column chromatography on silica gel (PE/EtAOc=1/1) to give methyl 6-((6-fluoro-2-methylpyridin-3-yl)oxy)-2-methyl-3-(1-methyl-1H-pyrazol-4-yl)benzoate (250 mg, 70.6%) as a yellow oil. LCMS (ESI) calcd. for C₁₉H₁₉FN₃O₃ [M+H]⁺ m/z 356.14, found 356.10.

Step 6: 6-((6-fluoro-2-methylpyridin-3-yl)oxy)-2-methyl-3-(1-methyl-1H-pyrazol-4-yl)benzoic acid: To a solution of methyl 6-((6-fluoro-2-methylpyridin-3-yl)oxy)-2-methyl-3-(1-methyl-1H-pyrazol-4-yl)benzoate (250 mg, 0.70 mmol) in MeOH/THF/H₂O (1/1/1, 9 mL) was added LiOH (168 mg, 7.04 mmol) at room temperature. The reaction mixture was heated at 50° C. for 12 hours. After the reaction was completed, the mixture was concentrated. The residue was adjusted to pH=3-4 with aqueous HCl (1M). Then the solution was extracted with EtOAc (30 mL×3). The combined organic layers were washed with brine, dried over sodium sulfate, concentrated under vacuum to provide 6-((6-fluoro-2-methylpyridin-3-yl)oxy)-2-methyl-3-(1-methyl-1H-pyrazol-4-yl)benzoic acid (90 mg, 37.5%) as a white solid. LCMS (ESI) calcd. for C₁₈H₁₇FN₃O₃ [M+H]⁺ m/z 342.13, found 342.05.

### Intermediate 4

### 6-((6-fluoro-2-methylpyridin-3-yl)oxy)-2-methyl-3-(trifluoromethyl)benzoic acid

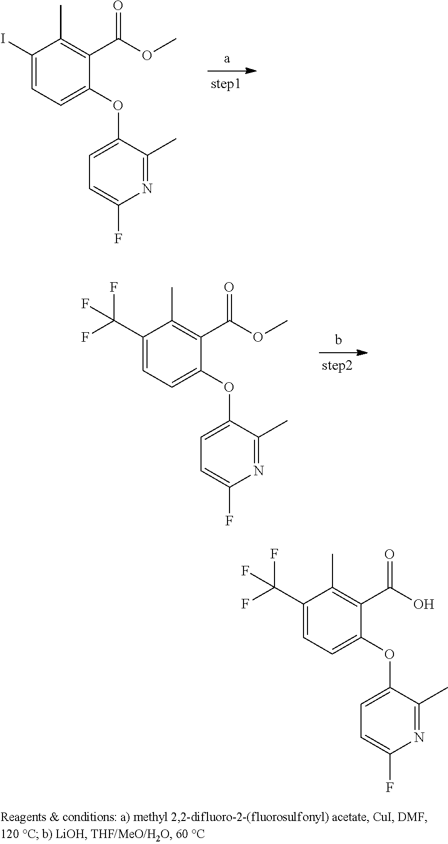 [SMILES: COC(=O)c1c(Oc2ccc(F)nc2C)ccc(I)c1C ; COC(=O)c1c(Oc2ccc(F)nc2C)ccc(C(F)(F)F)c1C ; Cc1nc(F)ccc1Oc1ccc(C(F)(F)F)c(C)c1C(=O)O]

Step 1: methyl 6-((6-fluoro-2-methylpyridin-3-yl)oxy)-2-methyl-3-(trifluoromethyl)benzoate: To a solution of methyl 6-((6-fluoro-2-methylpyridin-3-yl)oxy)-3-iodo-2-methylbenzoate (500 mg, 1.25 mmol) and CuI (475 mg, 2.49 mmol) in DMF (10 mL) was added methyl 2,2-difluoro-2-(fluorosulfonyl) acetate (1.20 g, 6.23 mmol) dropwise at room temperature under an atmosphere of N₂. The mixture was heated at 120° C. for 6 hours. After the reaction was completed, the resulting solution was diluted with water (50 mL) and extracted with EtOAc (50 mL×3). The combined organic phases were washed with brine, dried over sodium sulfate, concentrated under vacuum. The residue was purified by flash column chromatography on silica gel (PE/EtOAc=10/1 to 1/1) to give methyl 6-((6-fluoro-2-methylpyridin-3-yl)oxy)-2-methyl-3-(trifluoromethyl)benzoate (350 mg, 81.6%). LCMS (ESI) calcd. for C₁₆H₁₄F₄NO₃ [M+H]⁺ m/z 344.09, found 344.05.

Step 2: 6-((6-fluoro-2-methylpyridin-3-yl)oxy)-2-methyl-3-(trifluoromethyl)benzoic acid: To a solution of methyl 6-((6-fluoro-2-methylpyridin-3-yl)oxy)-2-methyl-3-(trifluoromethyl)benzoate (350 mg, 1.02 mmol) in MeOH/THF/H₂O (1/1/1, 9 mL) was added LiOH (244 mg, 10.20 mmol) at room temperature. The reaction mixture was heated at 60° C. for 12 hours. After the reaction was completed, the mixture was concentrated. The residue was adjusted to pH=3-4 with aqueous HCl (1M). Then the solution was extracted with EtOAc (30 mL×3). The combined organic layers were washed with brine, dried over sodium sulfate, concentrated under vacuum to provide 6-((6-fluoro-2-methylpyridin-3-yl)oxy)-2-methyl-3-(trifluoromethyl)benzoic acid (75 mg, 22.3%) as a white solid. LCMS (ESI) calcd. for C₁₅H₁₂F₄NO₃ [M+H]⁺ m/z 330.08, found 330.05.

### Intermediate 5

### 2-fluoro-6-((6-fluoro-2-methylpyridin-3-yl)oxy)-3-(trifluoromethyl)benzoic acid

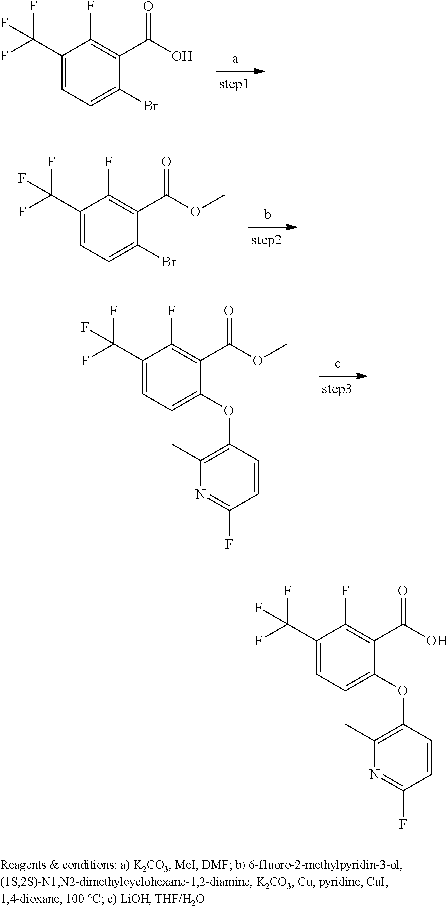 [SMILES: O=C(O)c1c(Br)ccc(C(F)(F)F)c1F ; COC(=O)c1c(Br)ccc(C(F)(F)F)c1F ; COC(=O)c1c(Oc2ccc(F)nc2C)ccc(C(F)(F)F)c1F ; Cc1nc(F)ccc1Oc1ccc(C(F)(F)F)c(F)c1C(=O)O]

Step 1: methyl 6-bromo-2-fluoro-3-(trifluoromethyl)benzoate: A mixture of 6-bromo-2-fluoro-3-(trifluoromethyl)benzoic acid (1 g, 3.48 mmol), K₂CO₃ (1.44 g, 10.44 mmol), MeI (988 mg, 6.96 mmol) in DMF (10 mL) was stirred at 25° C. for 16 hours. The resulting solution was diluted with water (80 mL) and extracted with EtOAc (50 mL×3). The combined organic layers were washed with brine, dried over Na₂SO₄, concentrated under vacuum. The residue was purified by flash column chromatography on silica gel (PE/EtOAc=3/1) to give methyl 6-bromo-2-fluoro-3-(trifluoromethyl)benzoate (950 mg, 90.9%) as a yellow solid. ¹H NMR (400 MHz, CD₃OD) δ 8.05-7.72 (m, 2H), 3.97 (s, 3H).

Step 2: methyl 2-fluoro-6-((6-fluoro-2-methylpyridin-3-yl)oxy)-3-(trifluoromethyl)benzoate: A solution of methyl 6-bromo-2-fluoro-3-(trifluoromethyl)benzoate (300 mg, 0.99 mmol), 6-fluoro-2-methylpyridin-3-ol (253 mg, 1.99 mmol), (1S,2S)—N1,N2-dimethylcyclohexane-1,2-diamine (28 mg, 0.20 mmol), K₂CO₃ (410 mg, 2.97 mmol), pyridine (235 mg, 2.97 mmol), Cu (13 mg, 0.20 mmol) and CuI (38 mg, 0.20 mmol) in 1,4-dioxane (10 mL) was heated at 100° C. for 16 hours under N2. LCMS showed the rection was completed. The mixture was diluted with water and extracted with EtOAc (3×50 mL). The combined organic layers were washed with brine, dried with Na₂SO₄, and concentrated under vacuum. The residue was purified by flash column chromatography on silica gel (PE/EtOAc=3/1) to give methyl 2-fluoro-6-((6-fluoro-2-methylpyridin-3-yl)oxy)-3-(trifluoromethyl)benzoate (250 mg, 72.5%) as a yellow oil. LCMS (ESI) calcd. for C₁₅H₁₁F₅NO₃ [M+H]⁺ m/z 348.07, found 348.00.

Step 3: 2-fluoro-6-((6-fluoro-2-methylpyridin-3-yl)oxy)-3-(trifluoromethyl)benzoic acid: To a solution of methyl 2-fluoro-6-((6-fluoro-2-methylpyridin-3-yl)oxy)-3-(trifluoromethyl)benzoate (250 mg, 0.72 mmol) in THF/H₂O (1/1, 10 mL) was added LiOH (173 mg, 7.2 mmol) at room temperature. The mixture was stirred at room temperature for 16 hours. After the reaction was completed, the mixture was concentrated to remove most THF. The aqueous phase was adjusted to pH=3-4 with 1N HCl then extracted with EtOAc (30 mL×3). The combined organic layers were washed with brine, dried with Na₂SO₄, concentrated under reduced pressure to give 2-fluoro-6-((6-fluoro-2-methylpyridin-3-yl)oxy)-3-(trifluoromethyl)benzoic acid (180 mg, 74.8%) as a yellow solid. LCMS (ESI) calcd. for C₁₄H₉F₅NO₃ [M+H]⁺ m/z 334.05, found 334.00.

### Intermediate 6

### 5-chloro-2-(4-cyano-2-methoxyphenoxy)-4-(trifluoromethyl)benzoic acid

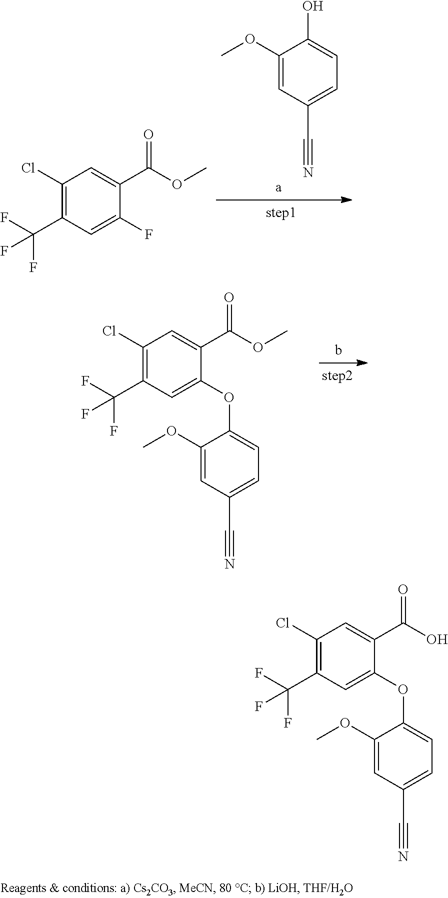 [SMILES: COC(=O)c1cc(Cl)c(C(F)(F)F)cc1F ; COc1cc(C#N)ccc1O ; COC(=O)c1cc(Cl)c(C(F)(F)F)cc1Oc1ccc(C#N)cc1OC ; COc1cc(C#N)ccc1Oc1cc(C(F)(F)F)c(Cl)cc1C(=O)O]

Step 1. methyl 5-chloro-2-(4-cyano-2-methoxyphenoxy)-4-(trifluoromethyl)benzoate: A solution of methyl 5-chloro-2-fluoro-4-(trifluoromethyl)benzoate (220 mg, 0.86 mmol), 4-hydroxy-3-methoxybenzonitrile (192 mg, 1.29 mmol) and Cs₂CO₃ (838 mg, 2.57 mmol) in ACN (5 mL) was heated to 80° C. and stirred for 3 hours. After the reaction was completed, the mixture was cooled to room temperature. The resulting solution was diluted with EtOAc (20 mL) and washed with water (20 mL×3). The combined organic layers were washed with brine, dried over sodium sulfate, concentrated under vacuum. The residue was purified by flash column chromatography on silica gel (PE/EtOAc=1/1) to provide methyl 5-chloro-2-(4-cyano-2-methoxyphenoxy)-4-(trifluoromethyl)benzoate (120 mg, 36.2% yield) as yellow solid. LCMS (ESI) calcd. for C₁₇H₁₂ClF₃NO₄ [M+H]⁻ m/z 386.14, found 386.0.

Step 2. 5-chloro-2-(4-cyano-2-methoxyphenoxy)-4-(trifluoromethyl)benzoic acid: A mixture of methyl 5-chloro-2-(4-cyano-2-methoxyphenoxy)-4-(trifluoromethyl)benzoate (120 mg, 0.31 mmol) in THF (2 mL) and H₂O (2 mL) was added LiOH (45 mg, 1.87 mmol). The mixture was stirred at room temperature for 4 hours. The reaction was monitored by LCMS. After the reaction was completed, the mixture was concentrated. The residue was adjusted to pH=3-4 with aqueous HCl (1 M). Then the solution was extracted with EtOAc (30 mL×3). The combined organic layers were washed with brine, dried over sodium sulfate, concentrated under vacuum to provide 5-chloro-2-(4-cyano-2-methoxyphenoxy)-4-(trifluoromethyl)benzoic acid (70 mg, 60.5% yield) as colorless oil which was used without further purification.

### Intermediate 7

### 4-cyclopropyl-6-((6-fluoro-2-methylpyridin-3-yl)oxy)-2-methyl-3-(trifluoromethyl)benzoic acid

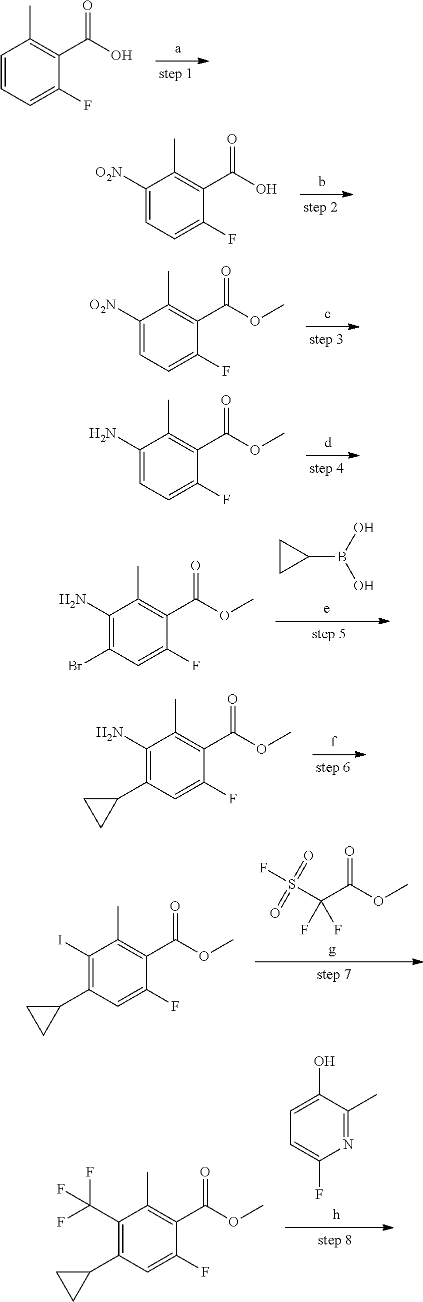

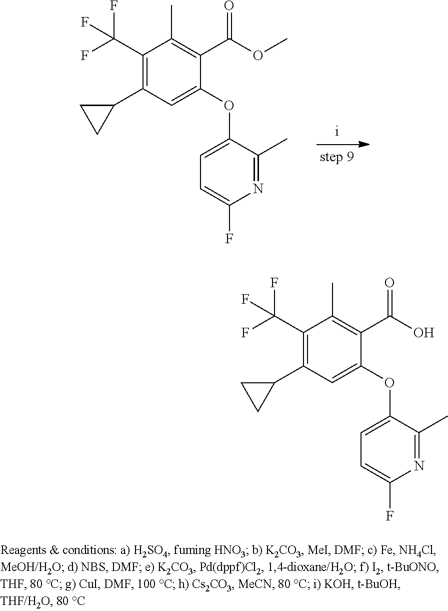 [SMILES: COC(=O)c1c(Oc2ccc(F)nc2C)cc(C2CC2)c(C(F)(F)F)c1C ; Cc1nc(F)ccc1Oc1cc(C2CC2)c(C(F)(F)F)c(C)c1C(=O)O]

Step 1. 6-fluoro-2-methyl-3-nitrobenzoic acid: A solution of 2-fluoro-6-methylbenzoic acid (3 g, 19.5 mmol) in H₂SO₄ (2 mL) was added H₂SO₄/fuming HNO₃ (6/1, 7 mL) at 0° C. and stirred for 5 hours at room temperature. Then the mixture was added to ice-cold water. The precipitate was collected by filtration and dried under vacuum to obtain 6-fluoro-2-methyl-3-nitrobenzoic acid (2.5 g, 64.4%) as a yellow solid. ¹H NMR (400 MHz, DMSO-d₆, ppm) δ 8.12 (dd, J=9.1, 5.2 Hz, 1H), 7.46 (t, J=8.8 Hz, 1H), 2.45 (s, 3H).

Step 2. methyl 6-fluoro-2-methyl-3-nitrobenzoate: A solution of 6-fluoro-2-methyl-3-nitrobenzoic acid (2.5 g, 12.6 mmol) and CH₃I (3.57 g, 25.1 mmol) in DMF (20 mL) was added K₂CO₃ (5.22 g, 37.8 mmol) and stirred at room temperature for 18 hours. The reaction was monitored by LCMS. After the reaction was completed, the resulting solution was diluted with water (50 mL) and extracted with EtOAc (50 mL×3). The combined organic layers were washed with brine, dried over Na₂SO₄, concentrated under vacuum. The residue was purified by flash column chromatography on silica gel (PE/EtOAc=1/1) to methyl 6-fluoro-2-methyl-3-nitrobenzoate (1.5 g, 55.9%) as a yellow solid.

Step 3. methyl 3-amino-6-fluoro-2-methylbenzoate: A solution of methyl 6-fluoro-2-methyl-3-nitrobenzoate (2 g, 9.39 mmol) in MeOH/H₂O (3:1, 8 mL) was added Fe powder (3.68 g, 65.7 mmol) and NH₄Cl (3.52 g, 65.7 mmol) at room temperature. The mixture was stirred 65° C. for 5 hours. The reaction was monitored by LCMS. After the reaction was completed, the resulting solution was diluted with water (50 mL) and extracted with EtOAc (50 mL×3). The combined organic layers were washed with brine, dried over Na₂SO₄, concentrated under vacuum. The residue was purified by flash column chromatography on silica gel (PE/EtOAc=1/1 to 1/3) to give methyl 3-amino-6-fluoro-2-methylbenzoate (1.5 g, 87.2%) as a yellow solid. LCMS (ESI) calcd. for C₉H₁₁FNO₂ [M+H]⁺ m/z 184.08, found 183.90.

Step 4. methyl 3-amino-4-bromo-6-fluoro-2-methylbenzoate: A solution of NBS (1.75 g, 9.8 mmol) in DMF (10 mL) was added methyl 3-amino-6-fluoro-2-methylbenzoate (1.5 g, 8.2 mmol) at 0° C. The mixture was stirred at room temperature for 3 hours. The reaction was monitored by LCMS. After the reaction was completed, the resulting solution was concentrated. The residue was purified by flash column chromatography on silica gel (PE/EtOAc=15/1 to 10/1) to give methyl 3-amino-4-bromo-6-fluoro-2-methylbenzoate (900 mg, 37.8%) as yellow oil. LCMS (ESI) calcd. for C₉H₁₀BrFNO₂ [M+H]⁺ m/z 261.99, found 216.95.

Step 5. methyl 3-amino-4-cyclopropyl-6-fluoro-2-methylbenzoate: A solution of cyclopropylboronic acid (590.0 mg, 6.87 mmol), Pd(dppf)Cl₂ (251.3 mg, 0.34 mmol) and K₂CO₃ (1.42 g, 10.3 mmol) in 1,4-dioxane/H₂O (4:1, 10 mL) was added methyl 3-amino-4-bromo-6-fluoro-2-methylbenzoate (900 mg, 3.43 mmol) at room temperature. The reaction mixture was heated at 100° C. for 16 hours under N2. After the reaction was completed, the resulting solution was diluted with water (50 mL) and extracted with EtOAc (50 mL×3). The combined organic layers were washed with brine, dried over Na₂SO₄, concentrated under vacuum. The residue was purified by flash column chromatography on silica gel (PE/EtOAc=10/1 to 5/1) to give methyl 3-amino-4-cyclopropyl-6-fluoro-2-methylbenzoate (700 mg, 73.1%) as yellow oil. LCMS (ESI) calcd. for C₁₂H₁₅FNO₂ [M+H]⁺ m/z 224.11, found 223.90.

Step 6. methyl 4-cyclopropyl-6-fluoro-3-iodo-2-methylbenzoate: A solution of methyl 3-amino-4-cyclopropyl-6-fluoro-2-methylbenzoate 6 (700 mg, 3.14 mmol) and 12 (1.19 g, 4.70 mmol) in THE (10 ml) was added t-BuONO (485.0 mg, 4.70 mmol) at room temperature. The reaction mixture was heated to 80° C. and refluxed for 2 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtOAc=15/1 to 10/1) to give methyl 4-cyclopropyl-6-fluoro-3-iodo-2-methylbenzoate (650 mg, 49.6%) as a black solid. LCMS (ESI) calcd. for C₁₂H₁₃FIO₂ [M+H]⁺ m/z 334.99, found 334.60.

Step 7. methyl 4-cyclopropyl-6-fluoro-2-methyl-3-(trifluoromethyl)benzoate: A solution of methyl 4-cyclopropyl-6-fluoro-3-iodo-2-methylbenzoate (650 mg, 1.95 mmol) and CuI (741.0 mg, 3.89 mmol) in DMF (10 ml) was added methyl 2,2-difluoro-2-(fluorosulfonyl)acetate (1.87 g, 9.73 mmol) at room temperature. The reaction mixture was heated at 100° C. for 5 hours under N2. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtOAc=10/1 to 5/1) to give methyl 4-cyclopropyl-6-fluoro-2-methyl-3-(trifluoromethyl)benzoate (500 mg, 83.7%) as yellow oil. LCMS (ESI) calcd. for C₁₃H₁₃F₄O₂ [M+H]⁺ m/z 277.08, found 277.10.

Step 8. methyl 4-cyclopropyl-6-((6-fluoro-2-methylpyridin-3-yl)oxy)-2-methyl-3-(trifluoromethyl)benzoate: A solution of methyl 4-cyclopropyl-6-fluoro-2-methyl-3-(trifluoromethyl)benzoate (500 mg, 1.81 mmol) and 6-fluoro-2-methylpyridin-3-ol (345.1 mg, 2.72 mmol) in MeCN (5 mL) was added Cs₂CO₃ (1.75 g, 5.43 mmol) at room temperature. The reaction mixture was heated at 80° C. for 5 hours. After the reaction was completed, the resulting solution was diluted with water (50 mL) and extracted with EtOAc (20 mL×3). The combined organic layers were washed with brine, dried over Na₂SO₄, concentrated under vacuum. The residue was purified by flash column chromatography on silica gel (PE/EtOAc=10/1 to 5/1) to give methyl 4-cyclopropyl-6-((6-fluoro-2-methylpyridin-3-yl)oxy)-2-methyl-3-(trifluoromethyl)benzoate (200 mg, 25.9%) as a white solid. LCMS (ESI) calcd. for C₁₉H₁₈F₄NO₃ [M+H]⁺ m/z 384.12, found 384.10.

Step 9. 4-cyclopropyl-6-((6-fluoro-2-methylpyridin-3-yl)oxy)-2-methyl-3-(trifluoromethyl)benzoic acid: A solution of methyl 4-cyclopropyl-6-((6-fluoro-2-methylpyridin-3-yl)oxy)-2-methyl-3-(trifluoromethyl)benzoate (200 mg, 0.81 mmol) in t-BuOH/THF/H₂O=1/1/1 (5 mL) was added KOH (1.46 g, 26.09 mmol) at room temperature. The reaction mixture was heated to 80° C. and stirred for 8 hours After the reaction was completed, the mixture was concentrated. The residue was adjusted to pH=3-4 with aqueous HCl (1M). Then the solution was extracted with EtOAc (30 mL×3). The combined organic layers were washed with brine, dried over Na₂SO₄, concentrated under vacuum to obtain 4-cyclopropyl-6-((6-fluoro-2-methylpyridin-3-yl)oxy)-2-methyl-3-(trifluoromethyl)benzoic acid (100 mg, 46.7%) as a yellow solid. LCMS (ESI) calcd. for C₁₈H₁₆F₄NO₃ [M+H]⁺ m/z 370.11, found 370.10.

### Intermediate 8

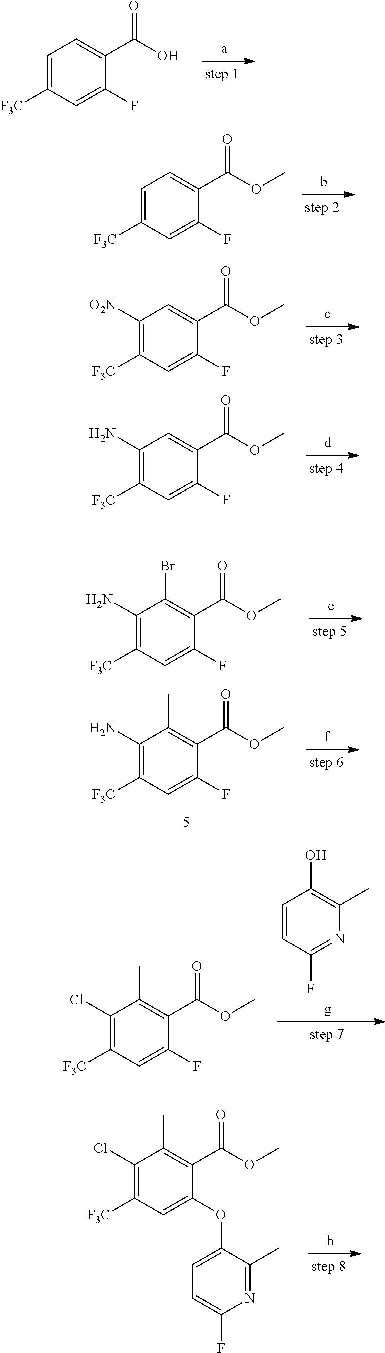

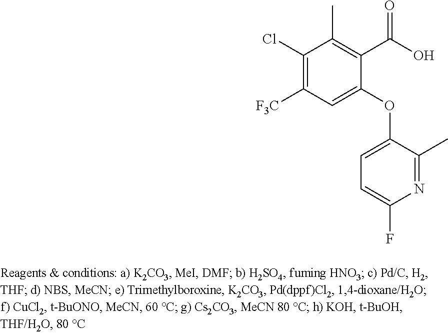 [SMILES: Cc1nc(F)ccc1Oc1cc(C(F)(F)F)c(Cl)c(C)c1C(=O)O]

Step 1. methyl 2-fluoro-4-(trifluoromethyl)benzoate: A solution of 2-fluoro-4-(trifluoromethyl)benzoic acid (8.5 g, 40.84 mmol) in DMF (100 mL) was added K₂CO₃ (16.91 g, 122.53 mmol) and CH₃I (8.6 g, 60.59 mmol). The mixture was stirred at room temperature for 16 hours. The filtrate was diluted with ice water (200 mL) and extracted with EtOAc (200 mL×3). The combined organic layers were washed with brine, dried over sodium sulfate, concentrated under vacuum, The residue was purified by flash column chromatography on silica gel (PE/EtOAc=10/1) to provide methyl 2-fluoro-4-(trifluoromethyl)benzoate (8 g, 85% yield) as white oil. LCMS (ESI) calcd. for C₉H₇F₄O₂ [M+H]⁺ m/z 223.04, found 223.04.

Step 2. methyl 2-fluoro-5-nitro-4-(trifluoromethyl)benzoate: A stirred solution of H₂SO₄ (80 mL) and fuming HNO₃ (6 mL) was added methyl 2-fluoro-4-(trifluoromethyl)benzoate 1 (8.0 g, 36.01 mmol) at 0° C. The mixture was heated to 50° C. and stirred for 5 hours. The mixture was poured into ice water (500 mL) and filtered. The filter cake was dried to provide methyl 2-fluoro-5-nitro-4-(trifluoromethyl)benzoate (4 g, 40% yield) as a yellow solid. ¹H NMR (400 MHz, CDCl₃, ppm) δ 8.58 (d, J=6.1 Hz, 1H), 7.65 (d, J=9.7 Hz, 1H), 4.02 (s, 3H).

Step 3. methyl 5-amino-2-fluoro-4-(trifluoromethyl)benzoate: A solution of methyl 2-fluoro-5-nitro-4-(trifluoromethyl)benzoate (4 g, 14.98 mmol) in THE (50 mL) was added Pd/C (600 mg) and stirred at room temperature for 16 hours under an atmosphere of H₂. LCMS showed the reaction was completed. The mixture was filtered through celite. The filtrate was diluted with water (100 mL) and extracted with EtOAc (100 mL×3). The combined organic layers were washed with brine, dried over sodium sulfate, concentrated under vacuum. The residue was purified by flash column chromatography on silica gel (PE/EtOAc=10/1) to provide methyl 5-amino-2-fluoro-4-(trifluoromethyl)benzoate (2.9 g, 81.69% yield) as a yellow solid. LCMS (ESI) calcd. for C₉H₈F₄NO₂ [M+H]⁺ m/z 238.05, found 237.60.

Step 4. methyl 3-amino-2-bromo-6-fluoro-4-(trifluoromethyl)benzoate: A solution of methyl 5-amino-2-fluoro-4-(trifluoromethyl)benzoate (2.9 g, 12.23 mmol) in MeCN (40 mL) was added NBS (3.05 g, 17.13 mmol) and stirred at room temperature for 1 hour. LCMS showed the reaction was completed. The mixture was diluted with water (100 mL) and extracted with EtOAc (100 mL×3). The combined organic layers were washed with brine, dried over sodium sulfate, concentrated under vacuum. The residue was purified by flash column chromatography on silica gel (PE/EtOAc=20/1) to provide methyl 3-amino-2-bromo-6-fluoro-4-(trifluoromethyl)benzoate (2.5 g, 64.94% yield) as light yellow oil. LCMS (ESI) calcd. for C₉H₇BrF₄NO₂ [M+H]⁺ m/z 315.96, found 315.90.

Step 5. methyl 3-amino-6-fluoro-2-methyl-4-(trifluoromethyl)benzoate: A solution of methyl 3-amino-2-bromo-6-fluoro-4-(trifluoromethyl)benzoate (2.5 g, 7.94 mmol), Trimethylboroxine (4.98 g, 39.69 mmol), K₂PO₃ (5.48 g, 39.69 mmol and Pd(dppf)Cl₂ (584 mg, 0.79 mmol) in dioxane (40 mL) and H₂O (10 mL) was heated to 100° C. and refluxed for 16 hours. LCMS showed the reaction was completed. The mixture was diluted with water (100 mL) and extracted with EtOAc (100 mL×3). The combined organic layers were washed with brine, dried over sodium sulfate, concentrated under vacuum. The residue was purified by flash column chromatography on silica gel (PE/EtOAc=20/1) to provide methyl 3-amino-6-fluoro-2-methyl-4-(trifluoromethyl)benzoate (1.5 g, 75.38% yield) as light yellow oil. LCMS (ESI) calcd. for C₁₀H₁₀F₄NO₂ [M+H]⁺ m/z 252.07, found 252.00.

Step 6. methyl 3-chloro-6-fluoro-2-methyl-4-(trifluoromethyl)benzoate: To a flame-dried round-bottomed flask equipped with a magnetic stir bar and addition funnel under N2 was added t-BuONO (1.22 g, 11.83 mmol), CuCl₂ (1.59 g, 11.83 mmol) and MeCN (20 mL). The solution was stirred at room temperature for 0.5 hour. A solution of methyl 3-amino-6-fluoro-2-methyl-4-(trifluoromethyl)benzoate (1.5 g, 5.74 mmol) in MeCN (10 mL) was added dropwise at 0° C. The reaction mixture was heated to 60° C. and stirred for 1 hour. LCMS showed the reaction was completed. The mixture was diluted with water (60 mL) and extracted with EtOAc (60 mL×3). The combined organic layers were washed with brine, dried over sodium sulfate, concentrated under vacuum. The residue was purified by flash column chromatography on silica gel (PE/EtOAc=20/1) to provide methyl 3-chloro-6-fluoro-2-methyl-4-(trifluoromethyl)benzoate (1.4 g, 87.50% yield) as colorless oil. ¹H NMR (400 MHz, DMSO-d₆, ppm) δ 7.88 (d, J=9.3 Hz, 1H), 3.96 (s, 3H), 2.40 (s, 3H).

Step 7. methyl 3-chloro-6-((6-fluoro-2-methylpyridin-3-yl)oxy)-2-methyl-4-(trifluoromethyl)benzoate: A solution of methyl 3-chloro-6-fluoro-2-methyl-4-(trifluoromethyl)benzoate (1.4 g, 5.17 mmol) and 6-fluoro-2-methylpyridin-3-ol (0.99 g, 7.79 mmol) in MeCN (30 mL) was added Cs₂CO₃ (6.78 g, 20.86 mmol) at room temperature. The mixture was heated to 80° C. and refluxed for 16 hours. After the reaction was completed, the mixture concentrated under vacuum and the residue was directly purified by flash column chromatography on silica gel (PE/EtOAc=10/1) to provide methyl 3-chloro-6-((6-fluoro-2-methylpyridin-3-yl)oxy)-2-methyl-4-(trifluoromethyl)benzoate (1 g, 51.28% yield) as colorless oil. LCMS (ESI) calcd. for C₁₆H₁₃ClF₄NO₃ [M+H]⁺ m/z 378.05, found 377.95.

Step 8. 3-chloro-6-((6-fluoro-2-methylpyridin-3-yl)oxy)-2-methyl-4-(trifluoromethyl)benzoic acid: A solution of methyl 3-chloro-6-((6-fluoro-2-methylpyridin-3-yl)oxy)-2-methyl-4-(trifluoromethyl)benzoate (800 mg, 2.12 mmol) in THF/t-BuOH/H₂O (1/1/1, 30 mL) was added KOH (1.54 g, 27.46 mmol) at 0° C. The reaction mixture was heated to 70° C. and refluxed for 16 hours. After the reaction was completed, the mixture was concentrated. The residue was adjusted to pH=3-4 with aqueous HCl (1M). Then the solution was extracted with EtOAc (60 mL×3). The combined organic layers were washed with brine, dried over sodium sulfate, concentrated under vacuum to provide 3-chloro-6-((6-fluoro-2-methylpyridin-3-yl)oxy)-2-methyl-4-(trifluoromethyl)benzoic acid (700 mg, 90.91% yield) as a white solid. LCMS (ESI) calcd. for C₁₅H₁₁ClF₄NO₃ [M+H]⁺ m/z 364.04, found 363.60.

### Intermediate 9

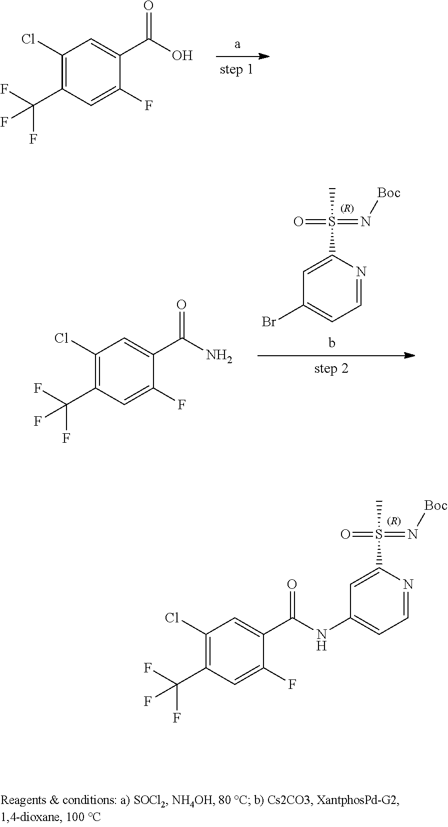

Step 1. 5-chloro-2-fluoro-4-(trifluoromethyl)benzamide: A solution of 5-chloro-2-fluoro-4-(trifluoromethyl)benzoic acid (200 mg, 0.82 mmol) in SOCl₂ (5 mL) was heated to 80° C. and stirred for 0.5 hour. The solution was concentrated under vacuum to provide the chloride intermediate. Then the chloride intermediate dissolved in THF (5 mL) was added to a stirred solution of THF (5 mL) in 28% NH₃·H₂O (5 mL) and at 0° C. The resulting mixture was stirred at 25° C. for 1 hour. Then the mixture was quenched with water (20 mL) and extracted with DCM (20 mL×3). The combined organic layers were washed with brine, dried over sodium sulfate, concentrated under vacuum. The residue was purified by flash column chromatography on silica gel (PE/EtOAc=1/1) to provide 5-chloro-2-fluoro-4-(trifluoromethyl)benzamide (180 mg, 90.36% yield) as yellow solid. LCMS (ESI) calcd. for C₈H₃ClF₄NO [M−H]⁻ m/z 239.98, found 240.1.

Step 2. tert-butyl (R)-((4-(5-chloro-2-fluoro-4-(trifluoromethyl)benzamido)pyridin-2-yl)(methyl)(oxo)-l6-sulfaneylidene)carbamate: A mixture of 5-chloro-2-fluoro-4-(trifluoromethyl)benzamide (500 mg, 2.07 mmol), tert-butyl (R)-((4-bromopyridin-2-yl)(methyl)(oxo)-l6-sulfaneylidene)carbamate (696 mg, 2.07 mmol), Cs₂CO₃ (1.75 g, 5.38 mmol) and Xantphos-Pd-G₂ (368 mg, 0.41 mmol) in 1,4-dioxane (10 mL) was heated at 100° C. for 2 hours under nitrogen. After the reaction was completed, the mixture was filtered through celite. The filtrate was concentrated and residue was purified by flash column chromatography on silica gel (PE/EtOAc=1/1) to provide tert-butyl (R)-((4-(5-chloro-2-fluoro-4-(trifluoromethyl)benzamido)pyridin-2-yl)(methyl)(oxo)-l6-sulfaneylidene)carbamate (400 mg, 38.89% yield) as a yellow solid. LCMS (ESI) calcd. for C₁₉H₁₇ClF₄N₃O₄S [M−H]⁻ m/z 494.05, found 494.0.

### Intermediate 10

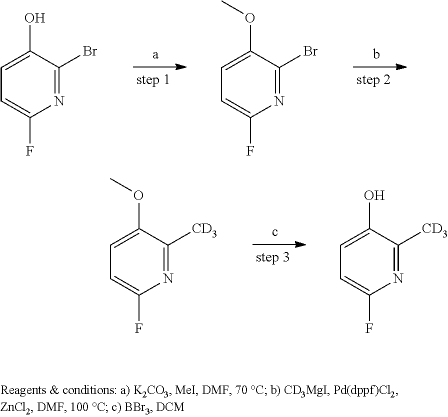

Step 1. 2-bromo-6-fluoro-3-methoxypyridine: A mixture of 2-bromo-6-fluoropyridin-3-ol 1 (1.5 g, 7.8 mmol), K₂CO₃ (3.23 g, 23.3 mmol), MeI (2.22 g, 15.6 mmol) in DMF (40 mL) was heated at 70° C. and stirred for 3 hours. The resulting solution was diluted with water (100 mL) and extracted with EtOAc (100 mL×3). The combined organic layers were washed with brine, dried over Na₂SO₄, concentrated under vacuum. The residue was purified by flash column chromatography on silica gel (PE/EtOAc=10/1) to give 2-bromo-6-fluoro-3-methoxypyridine (1.5 g, 93.3% yield) as a white solid. LCMS (ESI) calcd. for C₆H₆BrFNO [M+H]⁺ m/z 205.96, found 206.0.

Step 2. 6-fluoro-3-methoxy-2-(methyl-d3)pyridine: To a 250 mL 3-necked round-bottom flask purged and maintained with an inert atmosphere of nitrogen was added a solution of 2-bromo-6-fluoro-3-methoxypyridine (500 mg, 2.43 mmol), ZnCl₂ (4.96 g, 36.4 mmol) and Pd(dppf)Cl₂ (178 mg, 0.24 mmol) in DMF (10 mL). This was followed by the addition of CD₃MgI (1.0 M in diethyl ether, 36.4 mL) dropwise with stirring at room temperature. The solution was heated to 100° C. and stirred for 2 hours. The resulting solution was diluted with water (40 mL) and extracted with DCM (40 mL×3). The combined organic layers were dried over anhydrous sodium sulfate and concentrated under vacuum. The residue was purified by flash column chromatography on silica gel (PE/EtOAc=10/1) to give 6-fluoro-3-methoxy-2-(methyl-d3)pyridine (250 mg, 70.9% yield) as a white solid. LCMS (ESI) calcd. for C₇H₆D₃FNO [M+H]⁺ m/z 145.09, found 145.0.

Step 3. 6-fluoro-2-(methyl-d3)pyridin-3-ol: To a solution of 6-fluoro-3-methoxy-2-(methyl-d3)pyridine (250 mg, 1.73 mmol) in DCM (5 mL) was added BBr₃ (1.0 M in DCM, 1 mL) at room temperature. The reaction mixture was heated to 40° C. and stirred for 6 hours. LCMS showed the rection was completed. The mixture was concentrated under vacuum and quenched with CH₃OH (5 mL). The residue was purified by flash column chromatography on silica gel (PE/EtOAc=1/1) to give 6-fluoro-2-(methyl-d3)pyridin-3-ol 7 (60 mg, 26.7% yield) as a yellow solid. LCMS (ESI) calcd. for C₆H₄D₃FNO [M+H]⁺ m/z 131.07, found 131.0.

### Example 1A

### (R)-5-chloro-2-((6-fluoro-2-methylpyridin-3-yl)oxy)-N-(3-(S-methylsulfonimidoyl)phenyl)-4-(trifluoromethyl)benzamide

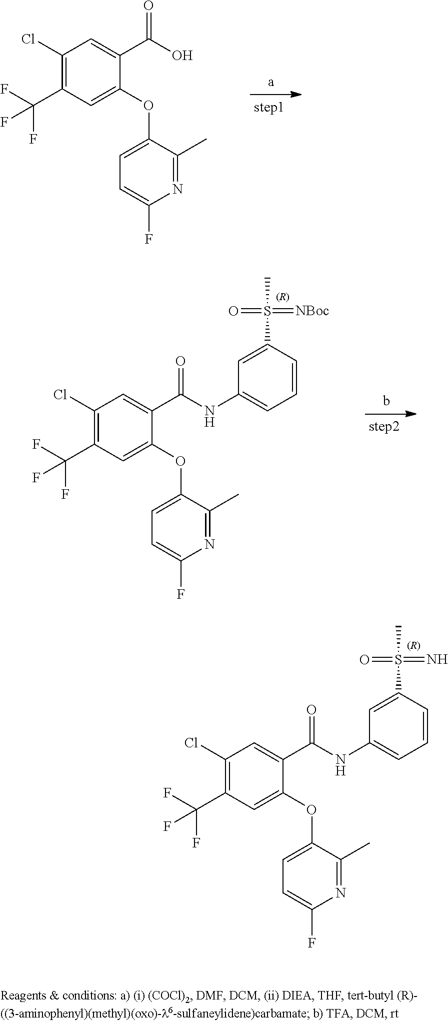

Step 1: tert-butyl (R)-((3-(5-chloro-2-((6-fluoro-2-methylpyridin-3-yl)oxy)-4-(trifluoromethyl)benzamido)phenyl)(methyl)(oxo)-λ⁶-sulfaneylidene)carbamate: To a solution of 5-chloro-2-((6-fluoro-2-methylpyridin-3-yl)oxy)-4-(trifluoromethyl)benzoic acid (120 mg, 0.34 mmol) in DCM (3 mL) oxalyl chloride (78 mg, 0.62 mmol) and DMF (20 μL) was added. The mixture was stirred at room temperature for 0.5 hours. Then the mixture was concentrated under vacuum. The residue was dissolved in THE (5 mL) and added to a solution of tert-butyl (R)-((3-aminophenyl)(methyl)(oxo)λ⁶-sulfaneylidene)carbamate (102 mg, 0.38 mmol) and DIEA (178 mg, 1.38 mmol) in THE (5 mL). The mixture was stirred at room temperature for 1 hour.

After the reaction was completed, the mixture was concentrated under vacuum. The residue was purified by flash column chromatography on silica gel (PE/EtOAc=2/1) to give tert-butyl (R)-((3-(5-chloro-2-((6-fluoro-2-methylpyridin-3-yl)oxy)-4-(trifluoromethyl)benzamido)phenyl)(methyl)(oxo)-λ⁶-sulfaneylidene)carbamate (110 mg, 53.4%) as a yellow oil. LCMS (ESI) calcd. for C₂₆H₂₅ClF₄N₃O₅S [M+H]⁺ m/z 602.12, found 602.00.

Step 2: (R)-5-chloro-2-((6-fluoro-2-methylpyridin-3-yl)oxy)-N-(3-(S-methylsulfonimidoyl)phenyl)-4-(trifluoromethyl)benzamide: A solution of tert-butyl (R)-((3-(5-chloro-2-((6-fluoro-2-methylpyridin-3-yl)oxy)-4-(trifluoromethyl) benzamido)phenyl)(methyl)(oxo)-λ⁶-sulfaneylidene)carbamate (110 mg, 0.18 mmol) in DCM (5 mL) was added TFA (0.5 mL) at room temperature. The reaction mixture was stirred at room temperature for 2 hours. After the reaction was completed, the solution was diluted with water (40 mL) and extracted with DCM (30 mL×3). The combined organic layers were washed with brine, dried over sodium sulfate, and concentrated under vacuum. The residue was purified by prep-HPLC (Gemini 5 um C₁₈ column, 150*21.2 mm, eluting with 45% to 95% MeCN/H₂O containing 0.1% NH₃) to obtain (R)-5-chloro-2-((6-fluoro-2-methylpyridin-3-yl)oxy)-N-(3-(S-methylsulfonimidoyl)phenyl)-4-(trifluoromethyl)benzamide (58.4 mg, 63.7%) as a white solid. ¹H NMR (400 MHz, DMSO-d₆) δ 10.96 (s, 1H), 8.29 (t, J=1.8 Hz, 1H), 8.10 (s, 1H), 7.82 (d, J=8.9 Hz, 1H), 7.71-7.63 (m, 2H), 7.58 (t, J=7.9 Hz, 1H), 7.37 (s, 1H), 7.04 (dd, J=8.7, 3.4 Hz, 1H), 4.22 (s, 1H), 3.04 (s, 3H), 2.34 (s, 3H). LCMS (ESI) calcd. for C₂₁H₁₇ClF₄N₃O₃S [M+H]⁺ m/z 502.06, found 502.00.

### Example 2A

### (R)-2-((6-fluoro-2-methylpyridin-3-yl)oxy)-N-(3-(S-methylsulfonimidoyl)phenyl)-4-(trifluoromethyl)benzamide

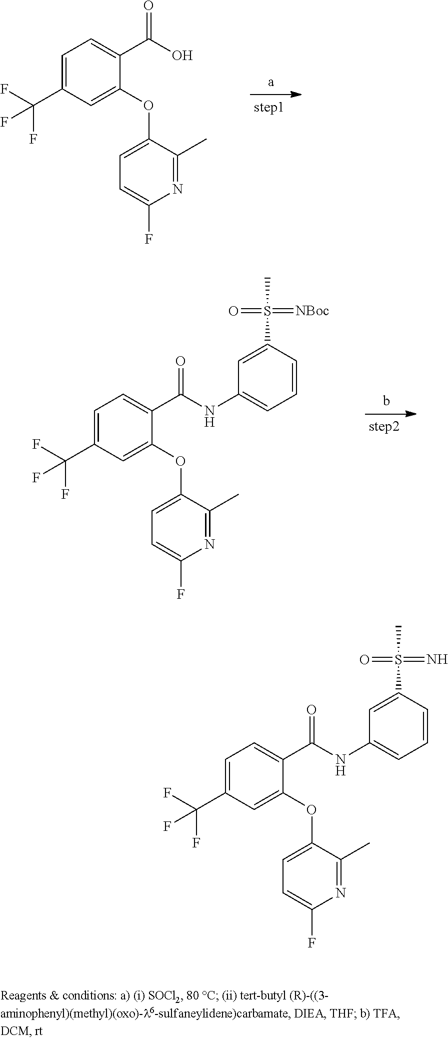

Step 1: tert-butyl (R)-((3-(2-((6-fluoro-2-methylpyridin-3-yl)oxy)-4-(trifluoromethyl)benzamido)phenyl)(methyl)(oxo)-λ⁶-sulfaneylidene)carbamate: A solution of 2-((6-fluoro-2-methylpyridin-3-yl)oxy)-4-(trifluoromethyl)benzoic acid (300 mg, 0.95 mmol) in SOCl₂ (5 mL) was stirred at 80° C. for 0.5 hour. Then the solution was concentrated under vacuum to provide the chloride intermediate. The chloride intermediate was added to a solution of tert-butyl (R)-((3-aminophenyl)(methyl)(oxo)-λ⁶-sulfaneylidene)carbamate (388 mg, 1.44 mmol) and DIEA (464.4 mg, 3.6 mmol) in THE (5 mL) at 0° C. The resulting mixture was stirred at 25° C. for 0.5 hour. Then the mixture was quenched with water (40 mL) and extracted with DCM (30 mL×2). The combined organic phases were washed with brine, dried over sodium sulfate, concentrated under vacuum. The residue was purified by flash column chromatography on silica gel (PE/EtOAc=3/1) to provide tert-butyl (R)-((3-(2-((6-fluoro-2-methylpyridin-3-yl)oxy)-4-(trifluoromethyl)benzamido)phenyl)(methyl)(oxo)-λ⁶-sulfaneylidene)carbamate (400 mg, 74.3%) as yellow solid. LCMS (ESI) calcd. for C₂₆H₂₆F₄N₃O₅S [M+H]⁺ m/z 568.16, found 568.10.

Step 2: (R)-2-((6-fluoro-2-methylpyridin-3-yl)oxy)-N-(3-(S-methylsulfonimidoyl)phenyl)-4-(trifluoromethyl)benzamide: A solution of tert-butyl (R)-((3-(2-((6-fluoro-2-methylpyridin-3-yl)oxy)-4-(trifluoromethyl)benzamido)phenyl)(methyl)(oxo)-λ⁶-sulfaneylidene)carbamate (400 mg, 0.70 mmol) in DCM (5 mL) TFA (0.5 mL) was added at room temperature. The reaction mixture was stirred at room temperature for 2 hours. After the reaction was completed, the solution was diluted with water (30 mL) and extracted with DCM (20 mL×3). The combine organic phases were washed with brine, dried over sodium sulfate, concentrated under vacuum. The residue was purified by prep-HPLC (Gemini 5 um C₁₈ column, 150*21.2 mm, eluting with 35% to 90% MeCN/H₂O containing 0.1% NH₃) to obtain (R)-2-((6-fluoro-2-methylpyridin-3-yl)oxy)-N-(3-(S-methylsulfonimidoyl)phenyl)-4-(trifluoromethyl)benzamide (125.4 mg, 42.5%) as a white solid. ¹H NMR (400 MHz, DMSO-d₆) δ 10.91 (s, 1H), 8.34 (s, 1H), 7.92-7.80 (m, 2H), 7.68-7.56 (m, 4H), 7.24 (s, 1H), 7.06 (dd, J=8.7, 3.4 Hz, 1H), 4.21 (s, 1H), 3.04 (s, 3H), 2.33 (s, 3H). LCMS (ESI) calcd. for C₂₁H₁₈F₄N₃O₃S [M+H]⁺ m/z 468.10, found 468.00.

### Example 3A

### (R)-6-((6-fluoro-2-methylpyridin-3-yl)oxy)-2-methyl-N-(3-(S-methylsulfonimidoyl)phenyl)-3-(trifluoromethyl)benzamide

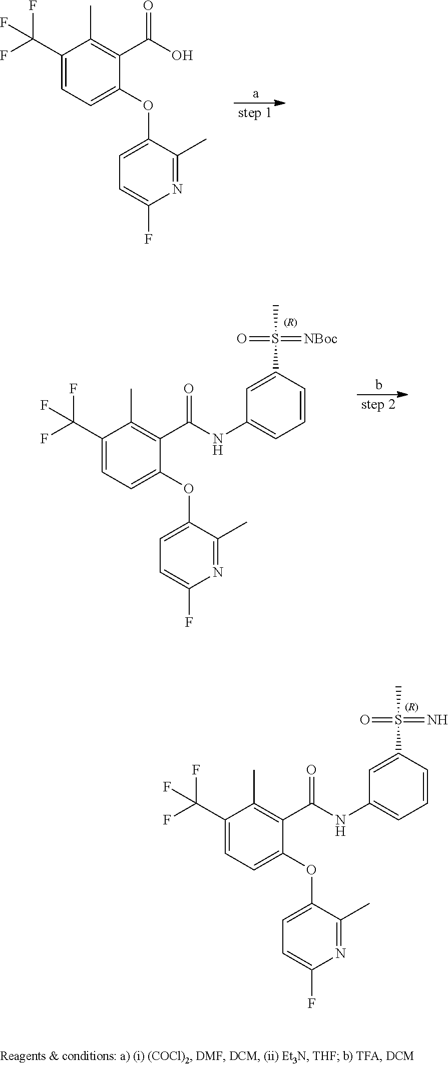

Step 1: tert-butyl (R)-((3-(6-((6-fluoro-2-methylpyridin-3-yl)oxy)-2-methyl-3-(trifluoromethyl)benzamido)phenyl)(methyl)(oxo)-λ⁶-sulfaneylidene)carbamate: To a solution of 6-((6-fluoro-2-methylpyridin-3-yl)oxy)-2-methyl-3-(trifluoromethyl)benzoic acid (75 mg, 0.23 mmol) in DCM (3 mL), oxalyl chloride (289 mg, 2.28 mmol) and DMF (20 L) was added. The mixture was stirred at room temperature for 0.5 hour. Then the mixture was concentrated under vacuum. The residue was dissolved in THF (5 mL) and added to a solution of tert-butyl (R)-((3-aminophenyl)(methyl)(oxo)-λ⁶-sulfaneylidene)carbamate (74 mg, 0.27 mmol) and Et₃N (115 mg, 1.14 mmol) in THF (5 mL). The mixture was stirred at room temperature for 1 hour. After the reaction was completed, the mixture was concentrated under vacuum. The residue was purified by flash column chromatography on silica gel (PE/EtOAc=2/1) to give tert-butyl (R)-((3-(6-((6-fluoro-2-methylpyridin-3-yl)oxy)-2-methyl-3-(trifluoromethyl)benzamido)phenyl)(methyl)(oxo)-λ⁶-sulfaneylidene)carbamate (55 mg, 41.5%) as a yellow oil. LCMS (ESI) calcd. for C₂₇H₂₈F₄N₃O₅S [M+H]⁺ m/z 582.17, found 582.10.

Step 2: (R)-6-((6-fluoro-2-methylpyridin-3-yl)oxy)-2-methyl-N-(3-(S-methylsulfonimidoyl)phenyl)-3-(trifluoromethyl)benzamide: A solution of tert-butyl (R)-((3-(6-((6-fluoro-2-methylpyridin-3-yl)oxy)-2-methyl-3-(trifluoromethyl)benzamido)phenyl)(methyl) (oxo)-λ⁶-sulfaneylidene)carbamate (55 mg, 0.09 mmol) in DCM (3 mL) was added TFA (0.3 mL) at room temperature. The reaction mixture was stirred at room temperature for 2 hours. After the reaction was completed, the mixture was concentrated. The residue was adjusted to pH of 8-9 with saturated aqueous NaHCO₃. The resulting solution was extracted with DCM (10 mL×3). The combined organic layers were washed with brine, dried over sodium sulfate, concentrated under vacuum. The residue was purified by prep-HPLC (Gemini 5 um C₁₈ column, 150*21.2 mm, eluting with 40% to 90% MeCN/H₂O containing 0.1% NH₄OH) to obtain (R)-6-((6-fluoro-2-methylpyridin-3-yl)oxy)-2-methyl-N-(3-(S-methylsulfonimidoyl)phenyl)-3-(trifluoromethyl)benzamide (15.6 mg, 34.3%) as a white solid. 1H NMR (400 MHz, DMSO-d₆) δ 11.08 (s, 1H), 8.41 (s, 1H), 7.86 (d, J=7.7 Hz, 1H), 7.79-7.65 (m, 3H), 7.58 (t, J=7.9 Hz, 1H), 7.12 (dd, J=8.7, 3.4 Hz, 1H), 6.74 (d, J=8.8 Hz, 1H), 4.22 (s, 1H), 3.06 (s, 3H), 2.45 (s, 3H), 2.28 (s, 3H). LCMS (ESI) calcd. for C₂₂H₂₀F₄N₃O₃S [M+H]⁺ m/z 482.12, found 482.05.

### Example 4A

### (R)-2-fluoro-6-((6-fluoro-2-methylpyridin-3-yl)oxy)-N-(3-(S-methylsulfonimidoyl)phenyl)-3-(trifluoromethyl)benzamide

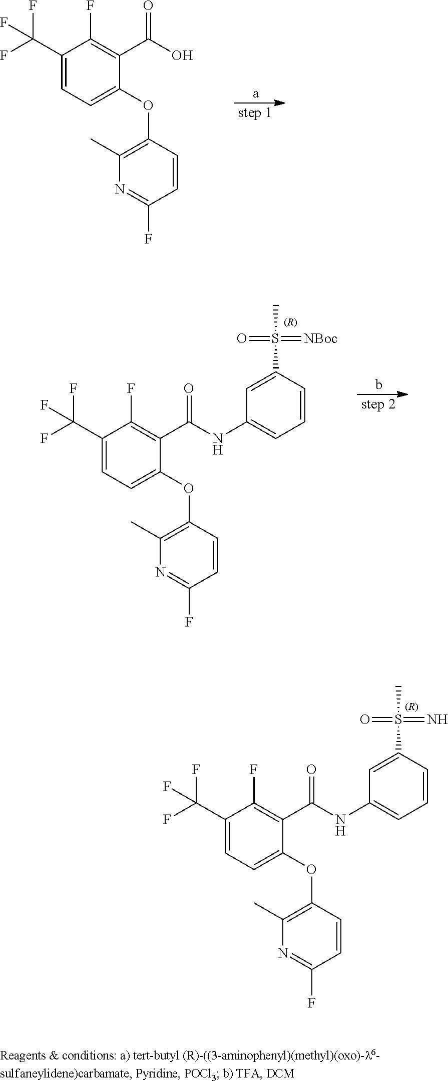

Step 1: tert-butyl (R)-((3-(2-fluoro-6-((6-fluoro-2-methylpyridin-3-yl)oxy)-3-(trifluoromethyl)benzamido)phenyl)(methyl)(oxo)-λ⁶-sulfaneylidene)carbamate: A mixture of 2-fluoro-6-((6-fluoro-2-methylpyridin-3-yl)oxy)-3-(trifluoromethyl)benzoic acid (150 mg, 0.45 mmol), tert-butyl (R)-((3-aminophenyl)(methyl)(oxo)-λ⁶-sulfaneylidene)carbamate (146 mg, 0.54 mmol) in pyridine (5 mL) was added POCl₃ (150 μL) dropwise at room temperature. The reaction solution was stirred at room temperature for 1 hour. Then the mixture was quenched with water (20 mL) and extracted with EtOAc (20 mL×2). The combined organic layers were washed with brine, dried over sodium sulfate, concentrated under vacuum. The residue was purified by flash column chromatography on silica gel (PE/EtOAc=1/1) to afford tert-butyl (R)-((3-(2-fluoro-6-((6-fluoro-2-methylpyridin-3-yl)oxy)-3-(trifluoromethyl)benzamido)phenyl)(methyl)(oxo)-λ⁶-sulfaneylidene)carbamate (120 mg, 45.5%) as a yellow solid. LCMS (ESI) calcd. for C₂₆H₂₅F₅N₃O₅S [M+H]⁺ m/z 586.15, found 586.10.

Step 2: (R)-2-fluoro-6-((6-fluoro-2-methylpyridin-3-yl)oxy)-N-(3-(S-methylsulfonimidoyl)phenyl)-3-(trifluoromethyl)benzamide: A solution of tert-butyl (R)-((3-(2-fluoro-6-((6-fluoro-2-methylpyridin-3-yl)oxy)-3-(trifluoromethyl)benzamido)phenyl)(methyl)(oxo)-λ⁶-sulfaneylidene)carbamate (120 mg, 0.20 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (30 mL×3). The combined organic phases were washed with brine, dried over sodium sulfate, concentrated under vacuum. The residue was purified by prep-HPLC (Gemini 5 um C₁₈ column, 150*21.2 mm, eluting with 40% to 95% McCN/H₂O containing 0.05% NH₃) to provide (R)-2-fluoro-6-((6-fluoro-2-methylpyridin-3-yl)oxy)-N-(3-(S-methylsulfonimidoyl)phenyl)-3-(trifluoromethyl)benzamide (70.50 mg, 72.5%) as a white solid. ¹H NMR (400 MHz, DMSO-d₆) δ 11.27 (s, 1H), 8.34 (d, J=1.7 Hz, 1H), 7.91-7.77 (m, 3H), 7.73-7.56 (m, 2H), 7.15 (dd, J=8.7, 3.4 Hz, 1H), 6.79 (d, J=8.9 Hz, 1H), 4.25 (s, 1H), 3.06 (s, 3H), 2.30 (s, 3H). LCMS (ESI) calcd. for C₂₁H₁₇F₅N₃O₃S [M+H]⁻ m/z 486.09, found 486.05.

### Example 5A and 6A

### (S)-5-chloro-2-((6-fluoro-2-methylpyridin-3-yl)oxy)-N-(2-(S-methylsulfonimidoyl)pyridin-4-yl)-4-(trifluoromethyl)benzamide and (R)-5-chloro-2-((6-fluoro-2-methylpyridin-3-yl)oxy)-N-(2-(S-methylsulfonimidoyl)pyridin-4-yl)-4-(trifluoromethyl)benzamide

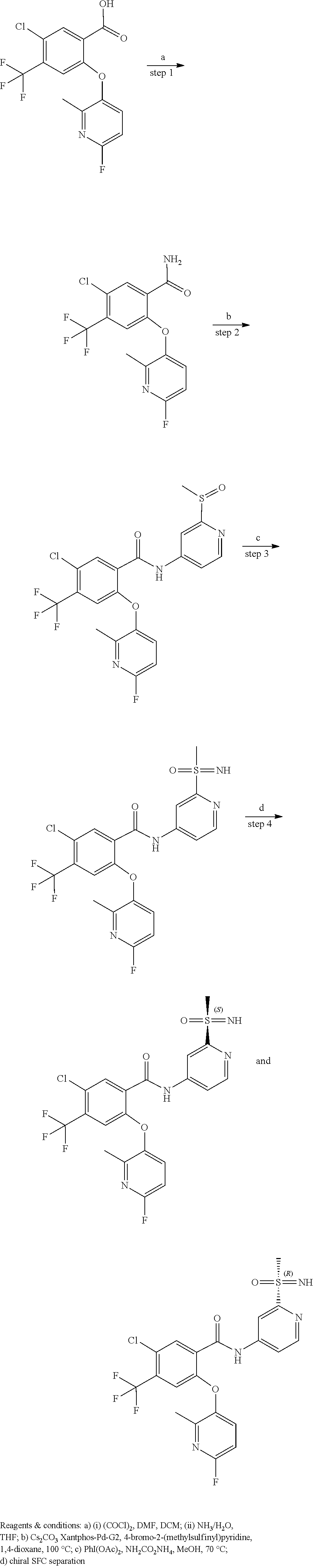

Step 1: 5-chloro-2-((6-fluoro-2-methylpyridin-3-yl)oxy)-4-(trifluoromethyl)benzamide: A solution of 5-chloro-2-((6-fluoro-2-methylpyridin-3-yl)oxy)-4-(trifluoromethyl)benzoic acid (150 mg, 0.43 mmol) in DCM (5 mL) was added oxalyl chloride (0.3 mL) and DMF (2 drops) at room temperature. After addition, the reaction mixture was stirred at room temperature for 1 hour. After the reaction was completed, the reaction mixture was concentrated. The residue was diluted with THE and added to a stirred solution of NH₃—H₂O (5 mL) dropwise. Then the mixture was stirred at room temperature for 1 hour. After the reaction was completed, the mixture was diluted with water (10 mL) and extracted with DCM (10 mL×3). The combine organic phases were washed with brine, dried over sodium sulfate, concentrated under vacuum. The residue was purified by flash column chromatography on silica gel (PE/EtOAc=1/1) to give 5-chloro-2-((6-fluoro-2-methylpyridin-3-yl)oxy)-4-(trifluoromethyl)benzamide (120 mg, 80.0%) as a white solid. LCMS (ESI) calcd. for C₁₄H₁₀ClF₄N₂O₂ [M+H]⁺ m/z 349.04, found 349.00.

Step 2: 5-chloro-2-((6-fluoro-2-methylpyridin-3-yl)oxy)-N-(2-(methylsulfinyl)pyridin-4-yl)-4-(trifluoromethyl)benzamide: A solution of 5-chloro-2-((6-fluoro-2-methylpyridin-3-yl)oxy)-4-(trifluoromethyl)benzamide (120 mg, 0.34 mmol) in 1,4-dioxane (8 mL) was added 4-bromo-2-(methylsulfinyl)pyridine (76 mg, 0.34 mmol), Cs₂CO₃ (292 mg, 0.89 mmol) and Xantphos-Pd-G2 (61 mg, 0.07 mmol) at room temperature. The reaction mixture was stirred at 100° C. for 16 hours under nitrogen. After the reaction was completed, the filtrate was diluted with water (20 mL) and extracted with DCM (20 mL×3). The combine organic phases were washed with brine, dried over sodium sulfate, concentrated under vacuum. The residue was purified by flash column chromatography on silica gel (PE/EtOAc=1/1) to give 5-chloro-2-((6-fluoro-2-methylpyridin-3-yl)oxy)-N-(2-(methylsulfinyl)pyridin-4-yl)-4-(trifluoromethyl)benzamide (105 mg, 62.1%) as a white solid. LCMS (ESI) calcd. for C₂₀H₁₅ClF₄N₃O₃S [M+H]⁺ m/z 488.05, found 488.05.

Step 3: 5-chloro-2-((6-fluoro-2-methylpyridin-3-yl)oxy)-N-(2-(S-methylsulfonimidoyl)pyridin-4-yl)-4-(trifluoromethyl)benzamide: To a solution of 5-chloro-2-((6-fluoro-2-methylpyridin-3-yl)oxy)-N-(2-(methylsulfinyl)pyridin-4-yl)-4-(trifluoromethyl)benzamide (105 mg, 0.22 mmol) in MeOH (5 mL) was added PhI(OAc)₂ (173 mg, 0.54 mmol) and ammonium carbamate (50 mg, 0.65 mmol) at room temperature. The reaction mixture was heated at 70° C. for 1 hour. After the reaction was completed, the mixture was cooled to room temperature, diluted with water (30 mL), and extracted with DCM (15 mL×3). The combine organic phases were washed with brine, dried over sodium sulfate, concentrated under vacuum. The residue was purified by flash column chromatography on silica gel (PE/EtOAc=1/1) to give the crude, then the residue was repurified by prep-HPLC (Gemini 5 um C₁₈ column, 150*21.2 mm, eluting with 30% to 90% MeCN/H₂O containing 0.1% formic acid) to give 5-chloro-2-((6-fluoro-2-methylpyridin-3-yl)oxy)-N-(2-(S-methylsulfonimidoyl)pyridin-4-yl)-4-(trifluoromethyl)benzamide (40.8 mg, 37.8%) as a white solid. LCMS (ESI) calcd. for C₂₀H₁₆ClF₄N₄O₃S [M+H]⁺ m/z 503.06, found 503.00.

Step 4: (S)-5-chloro-2-((6-fluoro-2-methylpyridin-3-yl)oxy)-N-(2-(S-methylsulfonimidoyl)pyridin-4-yl)-4-(trifluoromethyl)benzamide and (R)-5-chloro-2-((6-fluoro-2-methylpyridin-3-yl)oxy)-N-(2-(S-methylsulfonimidoyl)pyridin-4-yl)-4-(trifluoromethyl)benzamide: 5-chloro-2-((6-fluoro-2-methylpyridin-3-yl)oxy)-N-(2-(S-methylsulfonimidoyl)pyridin-4-yl)-4-(trifluoromethyl)benzamide was purified by Chiral-Prep-HPLC (Chiralpak-IC, 4.6*250 mm, eluting with 35% CO₂-MeOH containing 0.1% NH₃). The first eluting isomer was lyophilized to afford (S)-5-chloro-2-((6-fluoro-2-methylpyridin-3-yl)oxy)-N-(2-(S-methylsulfonimidoyl)pyridin-4-yl)-4-(trifluoromethyl)benzamide (12.7 mg) as a white solid. ¹H NMR (400 MHz, DMSO-d₆) δ 11.38 (s, 1H), 8.61 (d, J=5.6 Hz, 1H), 8.36 (s, 1H), 8.15 (s, 1H), 7.77-7.76 (m, 1H), 7.71-7.67 (m, 1H), 7.36 (s, 1H), 7.07-7.04 (m, 1H), 4.38 (s, 1H), 3.14 (s, 3H), 2.33 (s, 3H). LCMS (ESI) calcd. for C₂₀H₁₆ClF₄N₄O₃S [M+H]⁺ m/z 503.06, found 503.05 and the second eluting (R)-5-chloro-2-((6-fluoro-2-methylpyridin-3-yl)oxy)-N-(2-(S-methylsulfonimidoyl)pyridin-4-yl)-4-(trifluoromethyl)benzamide (14.5 mg) as a white solid. ¹H NMR (400 MHz, DMSO-d₆) δ 11.38 (s, 1H), 8.61 (d, J=5.6 Hz, 1H), 8.36 (s, 1H), 8.15 (s, 1H), 7.77-7.76 (m, 1H), 7.71-7.67 (m, 1H), 7.36 (s, 1H), 7.07-7.04 (m, 1H), 4.38 (s, 1H), 3.14 (s, 3H), 2.33 (s, 3H). LCMS (ESI) calcd. for C₂₀H₁₆ClF₄N₄O₃S [M+H]⁺ m/z 503.06, found 503.05.

### Example 7A

### (R)-6-((6-fluoro-2-methylpyridin-3-yl)oxy)-2-methyl-3-(1-methyl-1H-pyrazol-4-yl)-N-(3-(S-methylsulfonimidoyl)phenyl)benzamide

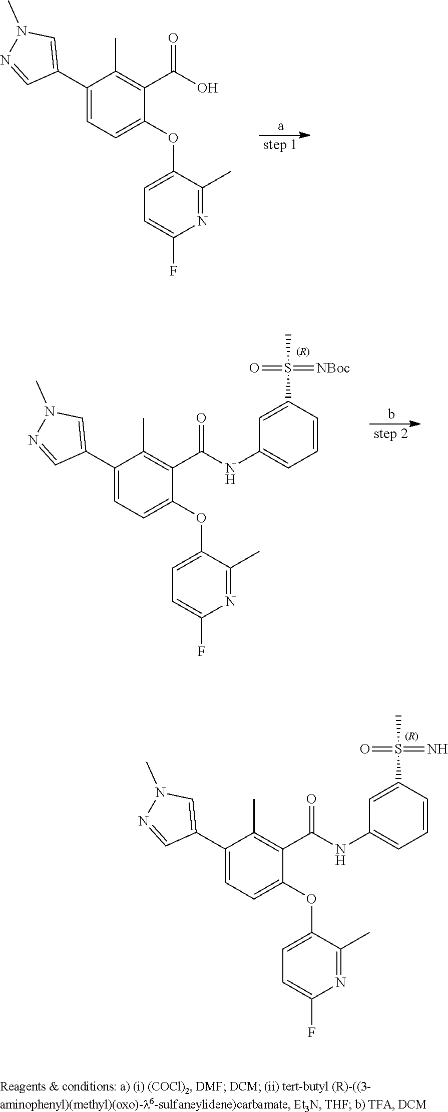

Step 1: tert-butyl (R)-((3-(6-((6-fluoro-2-methylpyridin-3-yl)oxy)-2-methyl-3-(1-methyl-1H-pyrazol-4-yl)benzamido)phenyl)(methyl)(oxo)-λ⁶-sulfaneylidene)carbamate: To a solution of 6-((6-fluoro-2-methylpyridin-3-yl)oxy)-2-methyl-3-(1-methyl-1H-pyrazol-4-yl)benzoic acid (90 mg, 0.26 mmol) in DCM (3 mL) was added oxalyl chloride (167 mg, 1.32 mmol) and DMF (20 μL). The mixture was stirred at room temperature for 0.5 hour. Then the mixture was concentrated under vacuum. The residue was dissolved in THE (5 mL) and added to a solution of tert-butyl (R)-((3-aminophenyl)(methyl)(oxo)-λ⁶-sulfaneylidene)carbamate (86 mg, 0.32 mmol) and TEA (133 mg, 1.32 mmol) in THE (5 mL). The mixture was stirred at room temperature for 1 hour. After the reaction was completed, the mixture was concentrated under vacuum. The residue was purified by flash column chromatography on silica gel (PE/EtOAc=2/1) to give tert-butyl (R)-((3-(6-((6-fluoro-2-methylpyridin-3-yl)oxy)-2-methyl-3-(1-methyl-1H-pyrazol-4-yl)benzamido)phenyl)(methyl)(oxo)-λ⁶-sulfaneylidene)carbamate (75 mg, 47.9%) as a yellow oil. LCMS (ESI) calcd. for C₃₀H₃₃FN₅O₅S [M+H]⁺ m/z 594.22, found 594.15.

Step 2: (R)-6-((6-fluoro-2-methylpyridin-3-yl)oxy)-2-methyl-3-(1-methyl-1H-pyrazol-4-yl)-N-(3-(S-methylsulfonimidoyl)phenyl)benzamide: A solution of tert-butyl (R)-((3-(6-((6-fluoro-2-methylpyridin-3-yl)oxy)-2-methyl-3-(1-methyl-1H-pyrazol-4-yl)benzamido)phenyl)(methyl)(oxo)-λ⁶-sulfaneylidene)carbamate (75 mg, 0.13 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (10 mL×3). The combine organic phases were washed with brine, dried over sodium sulfate, and concentrated under vacuum. The residue was purified by prep-HPLC (Gemini 5 um C₁₈ column, 150*21.2 mm, eluting with 40% to 90% McCN/H₂O containing 0.1% NH₄OH) to obtain (R)-6-((6-fluoro-2-methylpyridin-3-yl)oxy)-2-methyl-3-(1-methyl-1H-pyrazol-4-yl)-N-(3-(S-methylsulfonimidoyl)phenyl)benzamide (40.6 mg, 65.2% yield) as a white solid. ¹H NMR (400 MHz, DMSO-d₆) δ 10.96 (s, 1H), 8.42 (t, J=1.8 Hz, 1H), 7.93 (s, 1H), 7.85-7.80 (m, 1H), 7.66-7.52 (m, 4H), 7.36 (d, J=8.6 Hz, 1H), 7.05 (dd, J=8.7, 3.5 Hz, 1H), 6.68 (d, J=8.6 Hz, 1H), 4.20 (s, 1H), 3.89 (s, 3H), 3.05 (s, 3H), 2.35 (s, 3H), 2.31 (s, 3H). LCMS (ESI) calcd. for C₂₅H₂₅FN₅O₃S [M+H]⁺ m/z 494.17, found 494.05.

### Example 8A

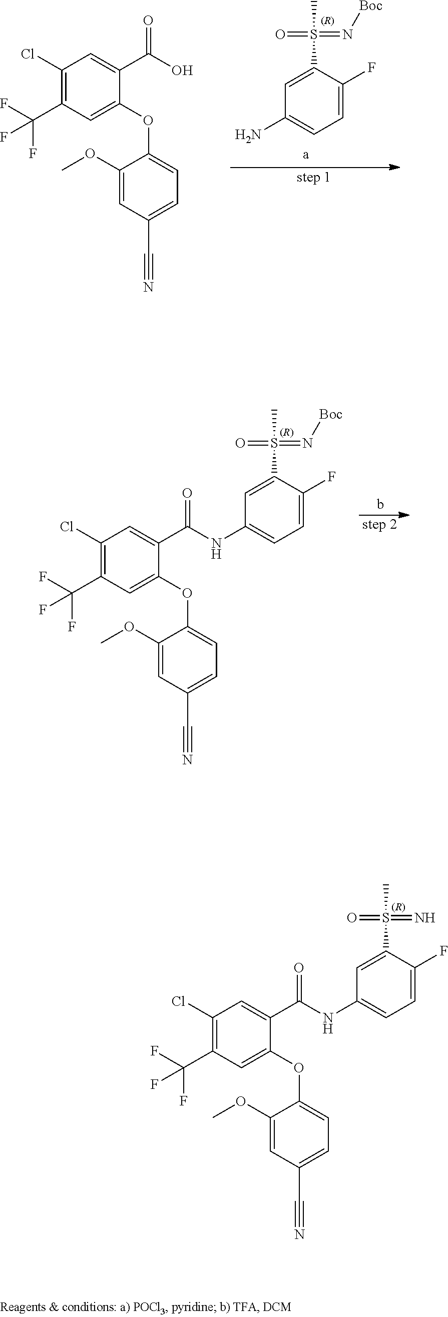

Step 1. tert-butyl (R)-((5-(5-chloro-2-(4-cyano-2-methoxyphenoxy)-4-(trifluoromethyl)benzamido)-2-fluorophenyl)(methyl)(oxo)-l6-sulfaneylidene)carbamate: A solution of 5-chloro-2-(4-cyano-2-methoxyphenoxy)-4-(trifluoromethyl)benzoic acid (60 mg, 0.16 mmol) and tert-butyl (R)-((5-amino-2-fluorophenyl)(methyl)(oxo)-16-sulfaneylidene)carbamate (70 mg, 0.24 mmol) in Pyridine (3 mL) was added POCl₃ (0.09 mL) at 0° C. The reaction mixture was stirred at 0° C. for 10 minutes. After the reaction was completed, the resulting solution was diluted with water (30 mL) and extracted with EtOAc (30 mL×3). The combined organic layers were washed with brine, dried over sodium sulfate, concentrated under vacuum. The residue was purified by flash column chromatography on silica gel (PE/EtOAc=1:1 to 1/2) to provide to tert-butyl (R)-((5-(5-chloro-2-(4-cyano-2-methoxyphenoxy)-4-(trifluoromethyl)benzamido)-2-fluorophenyl)(methyl)(oxo)-16-sulfaneylidene)carbamate (60 mg, 57.8% yield) as yellow solid. LCMS (ESI) calcd. for C₂₈H₂₅ClF₄N₃O₆S [M+H]⁺ m/z 642.11, found 642.1.

Step 2. (R)-5-chloro-2-(4-cyano-2-methoxyphenoxy)-N-(4-fluoro-3-(S-methylsulfonimidoyl)phenyl)-4-(trifluoromethyl)benzamide: A solution of tert-butyl (R)-((5-(5-chloro-2-(4-cyano-2-methoxyphenoxy)-4-(trifluoromethyl)benzamido)-2-fluorophenyl)(methyl)(oxo)-l6-sulfaneylidene)carbamate (60 mg, 0.09 mmol) in DCM (5 mL) was added TFA (1 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-HPLC (Gemini 5 um C₁₈ column, 150*21.2 mm, eluting with 20% to 65% MeCN/H₂O containing 0.05% NH₃·H₂O) to provide (R)-5-chloro-2-(4-cyano-2-methoxyphenoxy)-N-(4-fluoro-3-(S-methylsulfonimidoyl)phenyl)-4-(trifluoromethyl)benzamide (15 mg, 29.7% yield) as a white solid. ¹H NMR (400 MHz, DMSO-d₆, ppm) δ 10.91 (s, 1H), 8.19 (dd, J=6.5, 2.7 Hz, 1H), 8.08 (s, 1H), 7.90-7.82 (m, 1H), 7.63 (d, J=1.8 Hz, 1H), 7.47-7.38 (m, 3H), 7.17 (d, J=8.3 Hz, 1H), 4.68 (s, 1H), 3.79 (s, 3H), 3.18 (s, 3H). LCMS (ESI) calcd. for C₂₃H₁₇ClF₄N₃O₄S [M+H]⁺ m/z 542.06, found 542.1.

### Example 9A

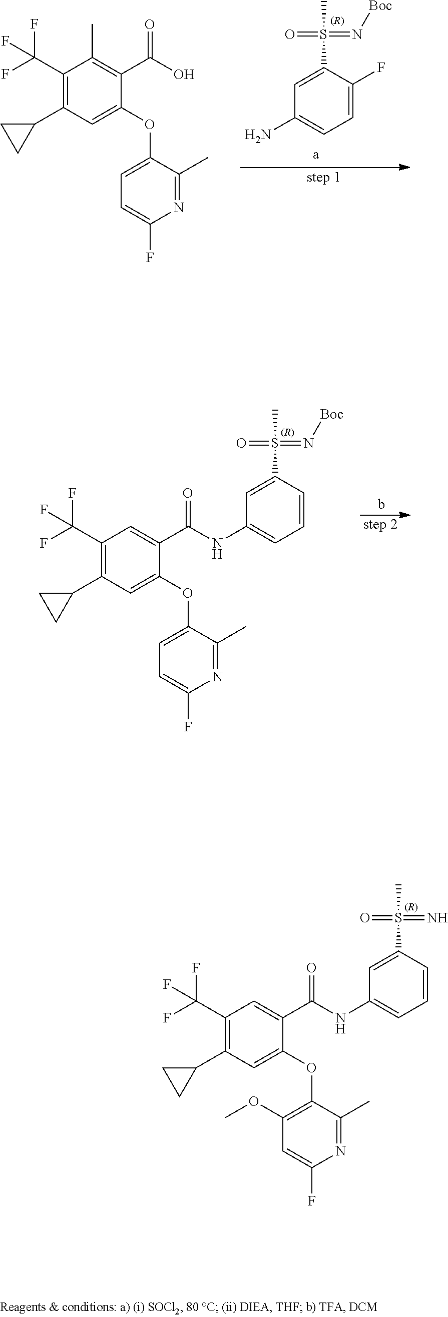

Step 1. tert-butyl (R)-((3-(4-cyclopropyl-6-((6-fluoro-2-methylpyridin-3-yl)oxy)-2-methyl-3-(trifluoromethyl)benzamido)phenyl)(methyl)(oxo)-l6-sulfaneylidene)carbamate: A solution of 4-cyclopropyl-6-((6-fluoro-2-methylpyridin-3-yl)oxy)-2-methyl-3-(trifluoromethyl)benzoic acid (100 mg, 0.27 mmol in SOCl₂ (2 ml) was heated for 1 hour at 80° C. The solution was concentrated under vacuum to provide the chloride intermediate. Then the chloride intermediate in THE (5 mL) was added to tert-butyl (R)-((3-aminophenyl)(methyl)(oxo)-l6-sulfaneylidene)carbamate (110.2 mg, 0.41 mmol) and DIEA (175.0 mg, 1.35 mmol) in THE (1 ml) at 0° C. and stirred for 2 hours at room temperature. After the reaction was completed, the mixture was concentrated under vacuum. The residue was purified by flash column chromatography on silica gel (PE/EtOAc=1/1 to 1/2) to give tert-butyl (R)-((3-(4-cyclopropyl-6-((6-fluoro-2-methylpyridin-3-yl)oxy)-2-methyl-3-(trifluoromethyl)benzamido)phenyl)(methyl)(oxo)-l6-sulfaneylidene)carbamate (130 mg, 69.4%) as a yellow solid. LCMS (ESI) calcd. for C₃₀H₃₂F₄N₃O₅S [M+H]⁺ m/z 622.20, found 622.10.

Step 2. (R)-4-cyclopropyl-6-((6-fluoro-2-methylpyridin-3-yl)oxy)-2-methyl-N-(3-(S-methylsulfonimidoyl)phenyl)-3-(trifluoromethyl)benzamide: A solution of tert-butyl (R)-((3-(4-cyclopropyl-6-((6-fluoro-2-methylpyridin-3-yl)oxy)-2-methyl-3-(trifluoromethyl)benzamido)phenyl)(methyl)(oxo)-l6-sulfaneylidene)carbamate (130 mg, 0.21 mmol) in DCM (2 mL) was added TFA (0.2 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (10 mL×3). The combined organic layers were washed with brine, dried over sodium sulfate, concentrated under vacuum. The residue was purified by prep-HPLC (Gemini 5 um C₁₈ column, 150*21.2 mm, eluting with 40% to 95% MeCN/H₂O containing 0.05% NH₃·H₂O) to obtain (R)-4-cyclopropyl-6-((6-fluoro-2-methylpyridin-3-yl)oxy)-2-methyl-N-(3-(S-methylsulfonimidoyl)phenyl)-3-(trifluoromethyl)benzamide (20 mg, 18.3% yield) as a white solid. ¹H NMR (400 MHz, DMSO-d₆, ppm) δ 11.00 (s, 1H), 8.39 (t, J=1.8 Hz, 1H), 7.86-7.78 (m, 1H), 7.72-7.63 (m, 2H), 7.57 (t, J=7.9 Hz, 1H), 7.08 (dd, J=8.7, 3.4 Hz, 1H), 6.44 (s, 1H), 4.21 (s, 1H), 3.05 (d, J=0.6 Hz, 3H), 2.48-2.41 (m, 3H), 2.28 (s, 3H), 2.23-2.13 (m, 1H), 0.98-0.87 (m, 2H), 0.55 (m, 2H). LCMS (ESI) calcd. for C₂₅H₂₄F₄N₃O₃S [M+H]⁺ m/z 522.15, found 522.20.

### Example 10A

### (R)-3-chloro-6-((6-fluoro-2-methylpyridin-3-yl)oxy)-2-methyl-N-(3-(S-methylsulfonimidoyl)phenyl)-4-(trifluoromethyl)benzamide

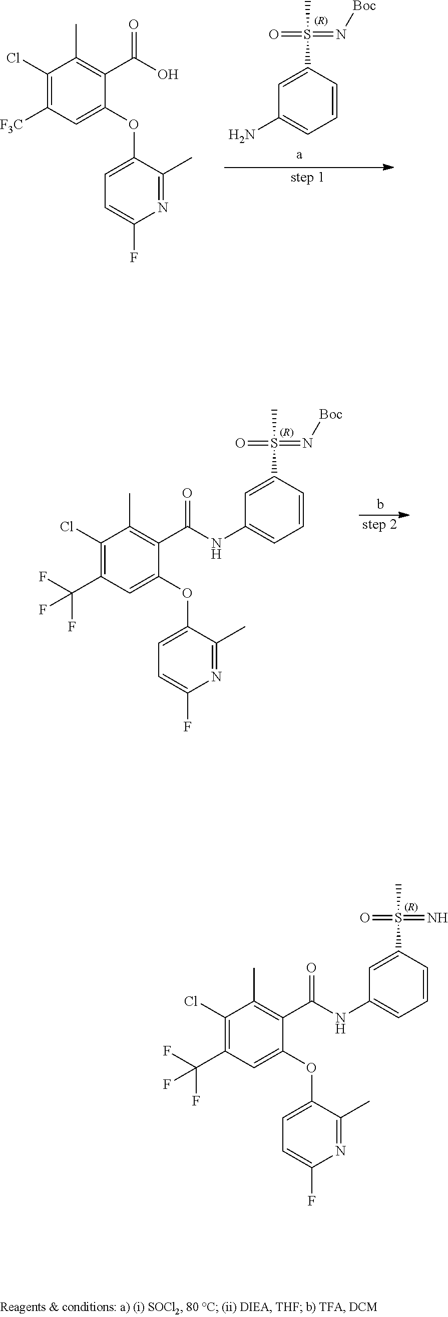

Step 1. tert-butyl (R)-((3-(3-chloro-6-((6-fluoro-2-methylpyridin-3-yl)oxy)-2-methyl-4-(trifluoromethyl)benzamido)phenyl)(methyl)(oxo)-l6-sulfaneylidene)carbamate: A solution of 3-chloro-6-((6-fluoro-2-methylpyridin-3-yl)oxy)-2-methyl-4-(trifluoromethyl)benzoic acid (150 mg, 0.41 mmol) in SOCl₂ (3 mL) was heated to 80° C. and stirred for 0.5 hour. The solution was concentrated under vacuum to provide the chloride intermediate. Then the chloride intermediate was added to a stirred solution of tert-butyl (R)-((3-aminophenyl)(methyl)(oxo)-16-sulfaneylidene)carbamate (112 mg, 0.41 mmol), DIEA (159 mg, 1.23 mmol) in THF (5 mL) at 0° C. The resulting mixture was stirred at 25° C. for 1 hour. Then the mixture was quenched with water (15 mL) and extracted with DCM (15 mL×3). The combined organic layers were washed with brine, dried over sodium sulfate, concentrated under vacuum. The residue was purified by flash column chromatography on silica gel (PE/EtOAc=3/2) to provide tert-butyl (R)-((3-(3-chloro-6-((6-fluoro-2-methylpyridin-3-yl)oxy)-2-methyl-4-(trifluoromethyl)benzamido)phenyl)(methyl)(oxo)-l6-sulfaneylidene)carbamate (180 mg, 70.82% yield) as light yellow oil. LCMS (ESI) calcd. for C₂₇H₂₆ClF₄N₃O₅SNa [M+Na]⁺ m/z 638.11, found 637.95.

Step 2. (R)-3-chloro-6-((6-fluoro-2-methylpyridin-3-yl)oxy)-2-methyl-N-(3-(S-methylsulfonimidoyl)phenyl)-4-(trifluoromethyl)benzamide: A solution of tert-butyl (R)-((3-(3-chloro-6-((6-fluoro-2-methylpyridin-3-yl)oxy)-2-methyl-4-(trifluoromethyl)benzamido)phenyl)(methyl)(oxo)-l6-sulfaneylidene)carbamate (180 mg, 0.29 mmol) in DCM (5 mL) was added TFA (0.5 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-HPLC (Gemini 5 um C₁₈ column, 150*21.2 mm, eluting with 40% to 95% MeCN/H₂O containing 0.05% NH₃·H₂O) to provide (R)-3-chloro-6-((6-fluoro-2-methylpyridin-3-yl)oxy)-2-methyl-N-(3-(S-methylsulfonimidoyl)phenyl)-4-(trifluoromethyl)benzamide (74.70 mg, 99.8% purity, 49.54% yield) as a white solid. ¹H NMR (400 MHz, DMSO-d₆, ppm) δ 11.08 (s, 1H), 8.31 (s, 1H), 7.80 (d, J=7.6 Hz, 1H), 7.69 (dd, J=14.8, 7.9 Hz, 2H), 7.57 (t, J=7.9 Hz, 1H), 7.16 (s, 1H), 7.06 (dd, J=8.7, 3.4 Hz, 1H), 4.20 (s, 1H), 3.05 (s, 3H), 2.45 (s, 3H), 2.30 (s, 3H). LCMS (ESI) calcd. for C₂₂H₁₉ClF₄N₃O₃S [M+H]⁺ m/z 516.08, found 516.00.

### Example 11A

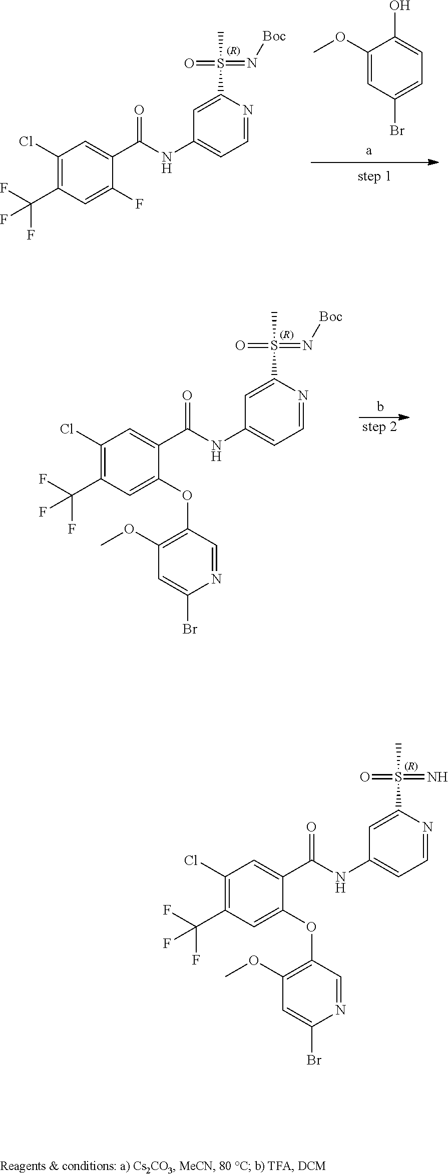

Step 1. tert-butyl (R)-((4-(2-(4-bromo-2-methoxyphenoxy)-5-chloro-4-(trifluoromethyl)benzamido)pyridin-2-yl)(methyl)(oxo)-l6-sulfaneylidene)carbamate: A mixture of tert-butyl (R)-((4-(5-chloro-2-fluoro-4-(trifluoromethyl)benzamido)pyridin-2-yl)(methyl)(oxo)-l6-sulfaneylidene)carbamate (200 mg, 0.40 mmol), 4-bromo-2-methoxyphenol (123 mg, 0.60 mmol), and Cs₂CO₃ (393 mg, 1.21 mmol) in MeCN (5 mL) was heated to 80° C. and stirred for 2 hours. After the reaction was completed, the resulting solution was diluted with water (20 mL) and extracted with EtOAc (20 mL×3). The combined organic layers were washed with brine, dried over Na₂SO₄, concentrated under vacuum. The residue was purified by flash column chromatography on silica gel (PE/EtOAc=1/1) to provide tert-butyl (R)-((4-(2-(4-bromo-2-methoxyphenoxy)-5-chloro-4-(trifluoromethyl)benzamido)pyridin-2-yl)(methyl)(oxo)-16-sulfaneylidene)carbamate (220 mg, 80.4% yield) as a yellow solid. LCMS (ESI) calcd. for C₂₆H₂₅BrClF₃N₃O₆S [M+H]⁺ m/z 678.03, found 680.1.

Step 2. (R)-2-(4-bromo-2-methoxyphenoxy)-5-chloro-N-(2-(S-methylsulfonimidoyl)pyridin-4-yl)-4-(trifluoromethyl)benzamide. A solution of tert-butyl (R)-((4-(2-(4-bromo-2-methoxyphenoxy)-5-chloro-4-(trifluoromethyl)benzamido)pyridin-2-yl)(methyl)(oxo)-l6-sulfaneylidene)carbamate (70 mg, 0.10 mmol) in DCM (5 mL) was added TFA (0.5 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.1% formic acid) to provide (R)-2-(4-bromo-2-methoxyphenoxy)-5-chloro-N-(2-(S-methylsulfonimidoyl)pyridin-4-yl)-4-(trifluoromethyl)benzamide (18 mg, 30.2% yield) as a white solid. ¹H NMR (400 MHz, DMSO-d₆, ppm) δ 11.31 (s, 1H), 8.62 (d, J=5.5 Hz, 1H), 8.39 (d, J=1.6 Hz, 1H), 8.08 (s, 1H), 7.80 (dd, J=5.4, 1.9 Hz, 1H), 7.37 (d, J=1.9 Hz, 1H), 7.25-7.04 (m, 3H), 4.37 (s, 1H), 3.75 (s, 3H), 3.14 (s, 3H). LCMS (ESI) calcd. for C₂₁H₁₇BrClF₃N₄O₄S [M+H]⁺ m/z 577.98, found 579.9.

### Example 12A

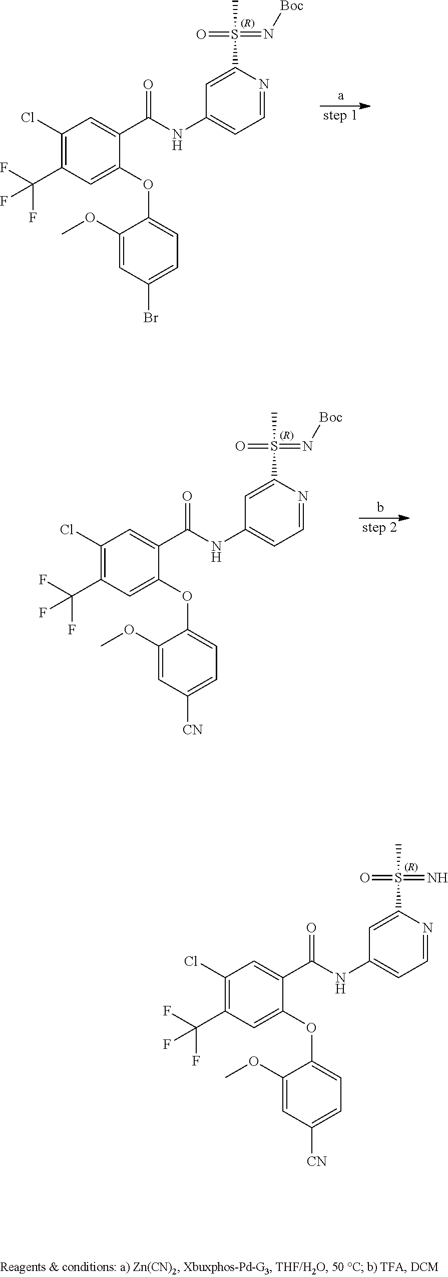

Step 1. tert-butyl (R)-((4-(5-chloro-2-(4-cyano-2-methoxyphenoxy)-4-(trifluoromethyl)benzamido)pyridin-2-yl)(methyl)(oxo)-l6-sulfaneylidene)carbamate: A mixture of tert-butyl (R)-((4-(2-(4-bromo-2-methoxyphenoxy)-5-chloro-4-(trifluoromethyl)benzamido)pyridin-2-yl)(methyl)(oxo)-l6-sulfaneylidene)carbamate (130 mg, 0.19 mmol), Zn(CN)₂ (34 mg, 0.29 mmol) and tBuXphos-Pd-G₃ (15 mg, 0.02 mmol) in THF/H₂O=4/1 (5 mL) was heated to 50° C. and stirred for 5 hours under a nitrogen atmosphere. After the reaction was completed, the resulting solution was diluted with water (20 mL) and extracted with EtOAc (20 mL×3). The combined organic layers were washed with brine, dried over Na₂SO₄, concentrated under vacuum. The residue was purified by flash column chromatography on silica gel (PE/EtOAc=1/1) to provide tert-butyl (R)-((4-(5-chloro-2-(4-cyano-2-methoxyphenoxy)-4-(trifluoromethyl)benzamido)pyridin-2-yl)(methyl)(oxo)-16-sulfaneylidene)carbamate (100 mg, 83.5% yield) as a yellow solid. LCMS (ESI) calcd. for C₂₇H₂₅ClF₃N₄O₆S [M+H]⁺ m/z 625.12, found 625.1.

Step 2. (R)-5-chloro-2-(4-cyano-2-methoxyphenoxy)-N-(2-(S-methylsulfonimidoyl)pyridin-4-yl)-4-(trifluoromethyl)benzamide: A solution of tert-butyl (R)-((4-(5-chloro-2-(4-cyano-2-methoxyphenoxy)-4-(trifluoromethyl)benzamido)pyridin-2-yl)(methyl)(oxo)-l6-sulfaneylidene)carbamate (100 mg, 0.16 mmol) in DCM (5 mL) was added TFA (0.5 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MCCN/H₂O containing 0.1% formic acid) to provide (R)-5-chloro-2-(4-cyano-2-methoxyphenoxy)-N-(2-(S-methylsulfonimidoyl)pyridin-4-yl)-4-(trifluoromethyl)benzamide (18 mg, 21.4% yield) as a white solid. ¹H NMR (400 MHz, DMSO-d₆, ppm) δ 11.34 (s, 1H), 8.61 (d, J=5.4 Hz, 1H), 8.33 (d, J=1.8 Hz, 1H), 8.14 (s, 1H), 7.75 (dd, J=5.4, 2.0 Hz, 1H), 7.63 (d, J=1.8 Hz, 1H), 7.52-7.38 (m, 2H), 7.20 (d, J=8.3 Hz, 1H), 4.37 (s, 1H), 3.78 (s, 3H), 3.14 (s, 3H). LCMS (ESI) calcd. for C₂₂H₁₇ClF₃N₄O₄S [M+H]⁺ m/z 525.06, found 525.0.

### Example 13A

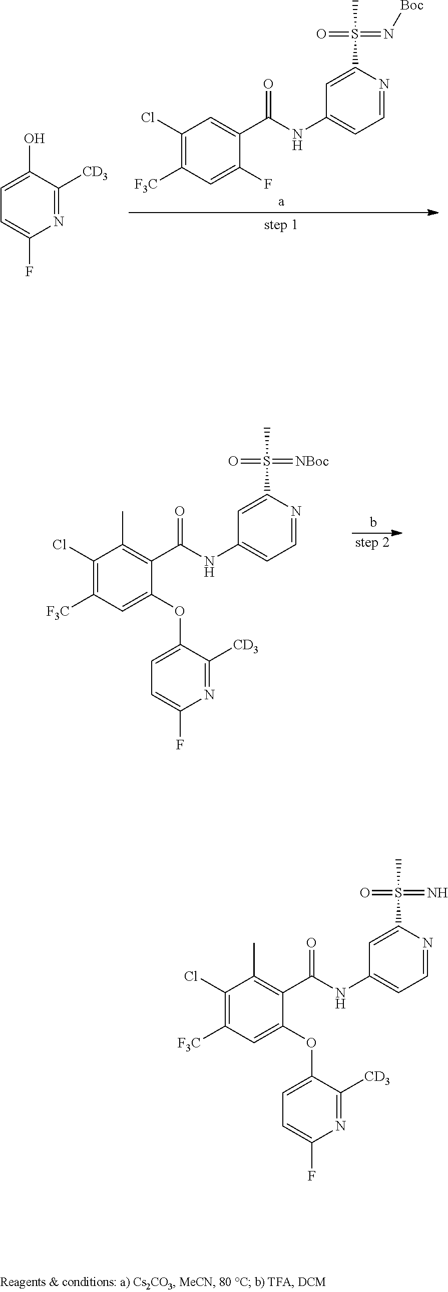

Step 1. tert-butyl (R)-((4-(5-chloro-2-((6-fluoro-2-(methyl-d3)pyridin-3-yl)oxy)-4-(trifluoromethyl)benzamido)pyridin-2-yl)(methyl)(oxo)-l6-sulfaneylidene)carbamate: A solution of 6-fluoro-2-(methyl-d3)pyridin-3-ol (26.2 mg, 0.20 mmol), tert-butyl (R)-((4-(5-chloro-2-fluoro-4-(trifluoromethyl)benzamido)pyridin-2-yl)(methyl)(oxo)-l6-sulfaneylidene)carbamate (100 mg, 0.20 mmol) and Cs₂CO₃ (196.8 mg, 0.60 mmol) in MeCN (6 mL) was heated to 80° C. and stirred for 6 hours. The mixture was cooled to room temperature, filtered through celite. The filtrate was concentrated and purified by Flash chromatography (PE/EA=5:1) to afford tert-butyl (R)-((4-(5-chloro-2-((6-fluoro-2-(methyl-d3)pyridin-3-yl)oxy)-4-(trifluoromethyl)benzamido)pyridin-2-yl)(methyl)(oxo)-l6-sulfaneylidene)carbamate (80 mg, 66.1% yield) as a white solid. LCMS (ESI) calcd. for C₂₅H₂₀D₃ClF₄N₄O₅SNa [M+Na]⁺ m/z 628.12, found 628.0.

Step 2. (R)-5-chloro-2-((6-fluoro-2-(methyl-d3)pyridin-3-yl)oxy)-N-(2-(S-methylsulfonimidoyl)pyridin-4-yl)-4-(trifluoromethyl)benzamide: A solution of tert-butyl (R)-((4-(5-chloro-2-((6-fluoro-2-(methyl-d3)pyridin-3-yl)oxy)-4-(trifluoromethyl)benzamido)pyridin-2-yl)(methyl)(oxo)-l6-sulfaneylidene)carbamate (80 mg, 0.13 mmol) in DCM (5 mL) was added TFA (1 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 55% to 60% MeCN/H₂O containing 0.05% NH₃·H₂O) to provide (R)-5-chloro-2-((6-fluoro-2-(methyl-d3)pyridin-3-yl)oxy)-N-(2-(S-methylsulfonimidoyl)pyridin-4-yl)-4-(trifluoromethyl)benzamide (27.00 mg, 41.1% yield) as a white solid. ¹H NMR (400 MHz, DMSO-d₆, ppm) δ 11.38 (s, 1H), 8.62 (d, J=5.4 Hz, 1H), 8.37 (d, J=1.5 Hz, 1H), 8.15 (s, 1H), 7.81-7.65 (m, 2H), 7.36 (s, 1H), 7.06 (dd, J=8.7, 3.4 Hz, 1H), 4.39 (s, 1H), 3.14 (s, 3H). LCMS (ESI) calcd. for C₂₀H₁₃D₃ClF₄N₄O₃S [M+H]⁺ m/z 506.08, found 506.0.

### Example 14 A

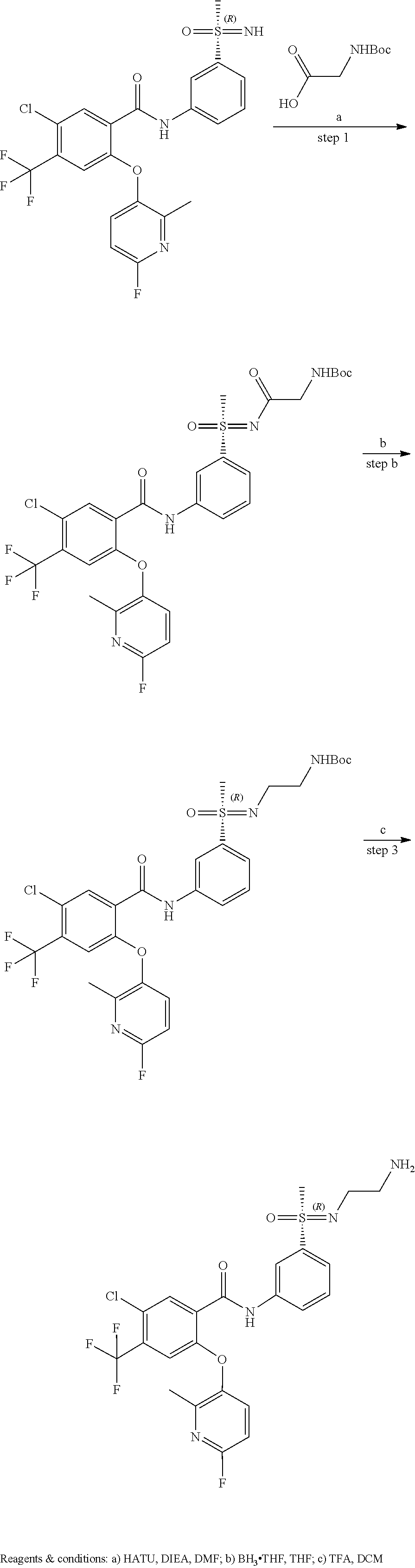

Step 1. tert-butyl (R)-(2-(((3-(5-chloro-2-((6-fluoro-2-methylpyridin-3-yl)oxy)-4-(trifluoromethyl)benzamido)phenyl)(methyl)(oxo)-l6-sulfaneylidene)amino)-2-oxoethyl)carbamate: A mixture of (R)-5-chloro-2-((6-fluoro-2-methylpyridin-3-yl)oxy)-N-(3-(S-methylsulfonimidoyl)phenyl)-4-(trifluoromethyl)benzamide (example 1) (120 mg, 0.24 mmol), (tert-butoxycarbonyl)glycine (83.8 mg, 0.48 mmol) and HATU (136.4 mg, 0.36 mmol) in DMF (5 mL) was added DIEA (92.7 mg, 0.72 mmol) at room temperature. The reaction mixture was stirred at room temperature for 16 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to provide tert-butyl (R)-(2-(((3-(5-chloro-2-((6-fluoro-2-methylpyridin-3-yl)oxy)-4-(trifluoromethyl)benzamido)phenyl)(methyl)(oxo)-l6-sulfaneylidene)amino)-2-oxoethyl)carbamate (100 mg, 63.5% yield) as a yellow solid. LCMS (ESI) calcd. for C₂₈H₂₈ClF₄N₄O₆S [M+H]⁺ m/z 659.14, found 659.10.

Step 2. tert-butyl (R)-(2-(((3-(5-chloro-2-((6-fluoro-2-methylpyridin-3-yl)oxy)-4-(trifluoromethyl)benzamido)phenyl)(methyl)(oxo)-l6-sulfaneylidene)amino)ethyl)carbamate: A solution of tert-butyl (R)-(2-(((3-(5-chloro-2-((6-fluoro-2-methylpyridin-3-yl)oxy)-4-(trifluoromethyl)benzamido)phenyl)(methyl)(oxo)-l6-sulfaneylidene)amino)-2-oxoethyl)carbamate (700 mg, 1.06 mmol) in THE (10 mL) was added BH₃-THF (0.7 mL) at 0° C. The reaction mixture was stirred at 0° C. for 1 hour under nitrogen. The reaction was monitored by LCMS. After the reaction was completed, the filtrate was diluted with water (20 mL) and extracted with DCM (20 mL×3). The combined organic layers were washed with brine, dried over sodium sulfate, concentrated under vacuum. The residue was purified by flash column chromatography on silica gel (PE/EtOAc=1/1) to give tert-butyl (R)-(2-(((3-(5-chloro-2-((6-fluoro-2-methylpyridin-3-yl)oxy)-4-(trifluoromethyl)benzamido)phenyl)(methyl)(oxo)-16-sulfaneylidene)amino)ethyl)carbamate (350 mg, 51.24% yield) as a white solid. LCMS (ESI) calcd. for C₂₈H₃₀ClF₄N₄O₅S [M+H]⁺ m/z 645.16, found 645.10.

Step 3. (R)—N-(3-(N-(2-aminoethyl)-S-methylsulfonimidoyl)phenyl)-5-chloro-2-((6-fluoro-2-methylpyridin-3-yl)oxy)-4-(trifluoromethyl)benzamide: A solution of tert-butyl (R)-(2-(((3-(5-chloro-2-((6-fluoro-2-methylpyridin-3-yl)oxy)-4-(trifluoromethyl)benzamido)phenyl)(methyl)(oxo)-l6-sulfaneylidene)amino)ethyl)carbamate (350 mg, 0.54 mmol) in DCM (10 mL) was added TFA (1 mL) at room temperature. The reaction mixture was stirred at room temperature for 2 hours. After the reaction was completed, the solution was quenched with saturated aqueous NaHCO₃ (20 mL) and extracted with DCM (20 mL×3). The combined organic layers were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% formic acid) to obtain (R)—N-(3-(N-(2-aminoethyl)-S-methylsulfonimidoyl)phenyl)-5-chloro-2-((6-fluoro-2-methylpyridin-3-yl)oxy)-4-(trifluoromethyl)benzamide formate (134.90 mg, 45.42% yield) as a white solid. ¹H NMR (400 MHz, DMSO-d₆, ppm) δ 11.06 (s, 1H), 8.33 (s, 1H), 8.25 (s, 1H), 8.10 (s, 1H), 7.83 (d, J=3.8 Hz, 1H), 7.67 (dd, J=8.6, 6.7 Hz, 1H), 7.64-7.60 (m, 2H), 7.38 (s, 1H), 7.04 (dd, J=8.8, 3.3 Hz, 1H), 3.17 (s, 3H), 2.77 (d, J=5.8 Hz, 4H), 2.34 (s, 3H). LCMS (ESI) calcd. for C₂₃H₂₂ClF₄N₄O₃S [M+H]⁺ m/z 545.11, found 545.00.

The following compounds are prepared using the techniques of the intermediates and examples described above.

| Molecule | | MS (m/z, | |
|---|---|---|---|
| Name | Structure | M + H)⁺ | NMR data |
| 15A | 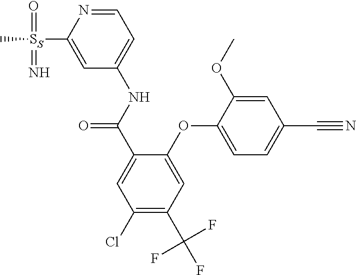 | 525 | ¹H NMR (400 MHz, DMSO-d₆, ppm) δ 11.33 (s, 1 H), 8.61 (d, J = 5.4 Hz, 1 H), 8.33 (s, 1 H), 8.14 (s, 1 H), 7.75 (dd, J = 5.4, 1.8 Hz, 1 H), 7.63 (d, J = 1.6 Hz, 1 H), 7.50-7.38 (m, 2 H), 7.20 (d, J = 8.3 Hz, 1 H), 4.38 (s, 1 H), 3.77 (s, 3 H), 3.14 (s, 3 H). |
| 16A | 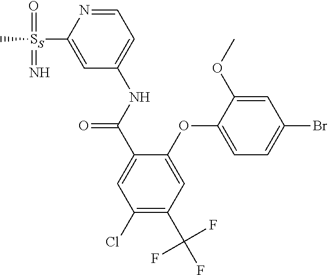 | 579.9 | ¹H NMR (400 MHz, DMSO-d₆, ppm) δ 11.31 (s, 1 H), 8.62 (d, J = 5.5 Hz, 1 H), 8.39 (d, J = 1.8 Hz, 1 H), 8.07 (s, 1 H), 7.80 (dd, J = 5.4, 2.0 Hz, 1 H), 7.37 (d, J = 2.0 Hz, 1 H), 7.24- 7.08 (m, 3 H), 4.37 (s, 1 H), 3.75 (s, 3 H), 3.14 (s, 3 H). |
| 17A | 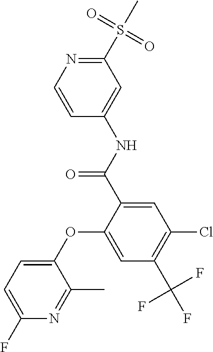 [SMILES: Cc1nc(F)ccc1Oc1cc(C(F)(F)F)c(Cl)cc1C(=O)Nc1ccnc(S(C)(=O)=O)c1] | 504.0 | ¹H NMR (400 MHz, DMSO-d₆, ppm) δ 11.45 (s, 1 H), 8.69 (d, J = 5.5 Hz, 1 H), 8.36 (d, J = 1.8 Hz, 1 H), 8.17 (s, 1 H), 7.84 (dd, J = 5.5, 1.9 Hz, 1 H), 7.69 (dd, J = 8.6, 6.8 Hz, 1 H), 7.37 (s, 1 H), 7.05 (dd, J = 8.8, 3.4 Hz, 1 H), 3.28 (s, 3 H), 2.33 (s, 3 H). |
| 18A | 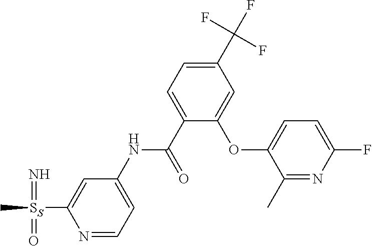 | 469 | ¹H NMR (400 MHz, DMSO-d₆, ppm) δ 11.33 (s, 1 H), 8.57 (d, J = 5.4 Hz, 1 H), 8.36 (s, 1 H), 7.91 (d, J = 7.9 Hz, 1 H), 7.75-7.62 (m, 3 H), 7.24 (s, 1 H), 7.05 (dd, J = 8.7, 3.4 Hz, 1 H), 4.31 (s, 1 H), 3.13 (s, 3 H), 2.33 (s, 3 H). |
| 19A | 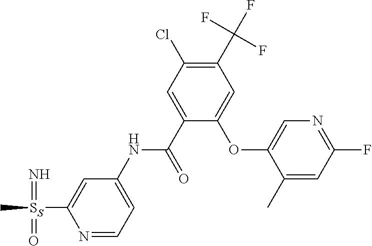 | 503 | ¹H NMR (400 MHz, DMSO-d₆, ppm) δ 11.37 (s, 1 H), 8.62 (d, J = 5.4 Hz, 1 H), 8.37 (d, J = 1.8 Hz, 1 H), 8.15 (s, 1 H), 7.96 (s, 1 H), 7.78 (dd, J = 5.4, 1.9 Hz, 1 H), 7.35 (s, 1 H), 7.21 (d, J = 1.8 Hz, 1 H), 4.38 (s, 1 H), 3.14 (s, 3 H), 2.25 (s, 3 H). |
| 20A | 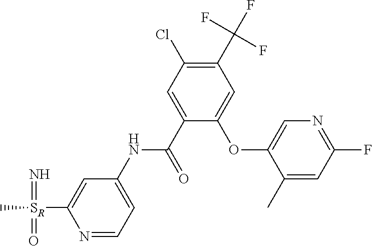 | 503 | ¹H NMR (400 MHz, DMSO-d₆, ppm) δ 11.38 (s, 1 H), 8.62 (d, J = 5.4 Hz, 1 H), 8.37 (s, 1 H), 8.15 (s, 1 H), 7.96 (s, 1 H), 7.77 (dd, J = 5.4, 1.3 Hz, 1 H), 7.35 (s, 1 H), 7.21 (s, 1 H), 4.38 (s, 1 H), 3.14 (s, 3 H), 2.25 (s, 3 H). |
| 21A | 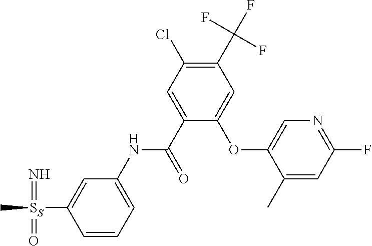 | 502.0 | ¹H NMR (400 MHz, DMSO-d₆, ppm) δ 10.96 (s, 1 H), 8.30 (s, 1 H), 8.10 (s, 1 H), 7.95 (s, 1 H), 7.83 (d, J = 8.0 Hz, 1 H), 7.68 (d, J = 7.9 Hz, 1 H), 7.58 (t, J = 7.9 Hz, 1 H), 7.35 (s, 1 H), 7.20 (d, J = 1.7 Hz, 1 H), 4.22 (s, 1 H), 3.33 (s, 3 H), 2.26 (s, 3 H). |
| 22A | 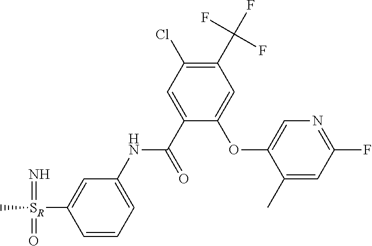 | 502.0 | ¹H NMR (400 MHz, DMSO-d₆, ppm) δ 10.96 (s, 1 H), 8.28 (s, 1 H), 8.09 (s, 1 H), 7.94 (s, 1 H), 7.81 (d, J = 8.5 Hz, 1 H), 7.66 (d, J = 8.1 Hz, 1 H), 7.57 (t, J = 7.9 Hz, 1 H), 7.35 (s, 1 H), 7.20 (d, J = 1.9 Hz, 1 H), 4.20 (s, 1 H), 3.04 (s, 3 H), 2.25 (s, 3 H). |
| 23A | 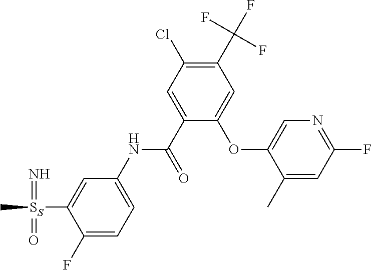 | 519.9 | ¹H NMR (400 MHz, DMSO-d₆, ppm) δ 10.96 (s, 1 H), 8.23 (dd, J = 6.4, 2.7 Hz, 1 H), 8.09 (s, 1 H), 7.94 (s, 1 H), 7.90- 7.84 (m, 1 H), 7.43 (t, J = 9.3 Hz, 1 H), 7.34 (s, 1 H), 7.21 (d, J = 1.8 Hz, 1 H), 4.69 (s, 1 H), 3.20 (s, 3 H), 2.25 (s, 3 H). |
| 24A | 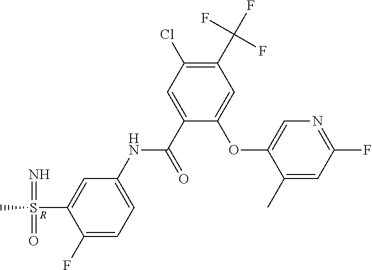 | 520.0 | ¹H NMR (400 MHz, DMSO-d₆, ppm) δ 10.96 (s, 1 H), 8.21 (dd, J = 6.4, 2.6 Hz, 1 H), 8.08 (s, 1 H), 7.92 (s, 1 H), 7.88- 7.82 (m, 1 H), 7.41 (t, J = 9.3 Hz, 1 H), 7.34 (s, 1 H), 7.20 (d, J = 1.9 Hz, 1 H), 4.68 (s, 1 H), 3.18 (s, 3 H), 2.25 (s, 3 H). |
| 25A | 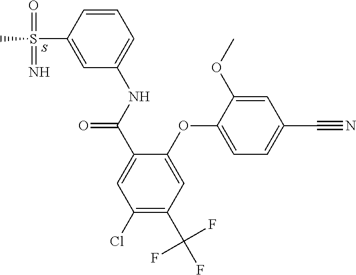 | 524.0 | ¹H NMR (400 MHz, DMSO-d₆, ppm) δ 11.00 (s, 1 H), 7.89- 7.86 (m, 2 H), 7.62- 7.50 (m, 6 H), 7.05 (s, 1 H), 4.29 (s, 1 H), 3.81 (s, 3 H), 3.07 (s, 3 H). |
| 26A | 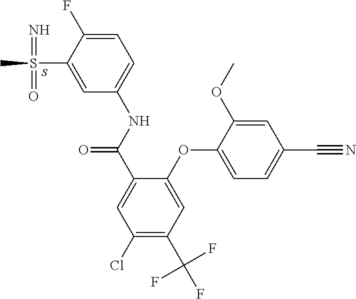 | 542.0 | ¹H NMR (400 MHz, DMSO-d₆, ppm) δ 11.01 (s, 1 H), 7.82 (s, 1 H), 7.67-7.35 (m, 6 H), 7.05 (s, 1 H), 4.84 (s, 1 H), 3.81 (s, 3 H), 3.20 (s, 3 H). |
| 27A | 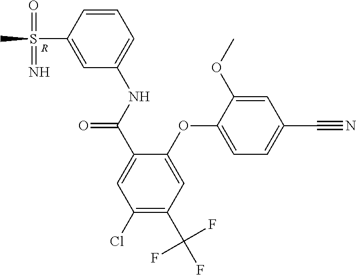 | 524.0 | ¹H NMR (400 MHz, DMSO-d₆, ppm) δ 11.04 (s, 1H), 7.98- 7.75 (m, 2 H), 7.69- 7.33 (m, 6 H), 7.03 (s, 1 H), 4.26 (s, 1 H), 3.81 (s, 3 H), 3.03 (s, 3 H). |
| 28A | 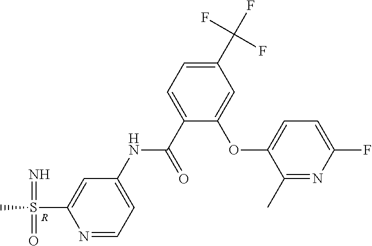 | 469.0 | ¹H NMR (400 MHz, DMSO-d₆, ppm) δ 11.33 (s, 1 H), 8.59 (d, J = 5.4 Hz, 1 H), 8.38 (d, J = 1.6 Hz, 1 H), 7.92 (d, J = 7.9 Hz, 1 H), 7.79-7.64 (m, 3 H), 7.24 (s, 1 H), 7.06 (dd, J = 8.7, 3.4 Hz, 1 H), 4.33 (s, 1 H), 3.13 (s, 3 H), 2.33 (s, 3 H). |
| 29A | 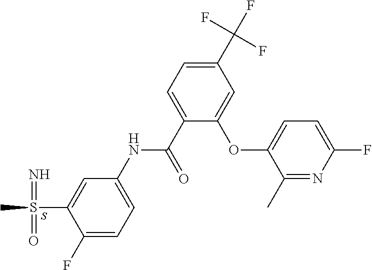 | 486.1 | ¹H NMR (400 MHz, DMSO-d₆, ppm) δ 10.90 (s, 1 H), 8.27 (dd, J = 6.5, 2.7 Hz, 1 H), 8.01-7.78 (m, 2 H), 7.78-7.54 (m, 2 H), 7.42 (t, J = 9.3 Hz, 1 H), 7.23 (s, 1 H), 7.06 (dd, J = 8.7, 3.4 Hz, 1 H), 4.67 (s, 1 H), 3.18 (s, 3 H), 2.33 (s, 3 H). |
| 30A | 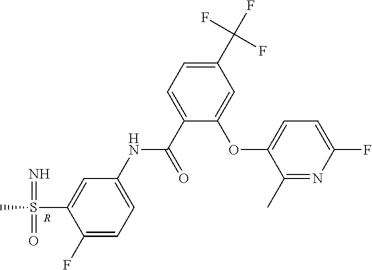 | 486.0 | ¹H NMR (400 MHz, DMSO-d₆, ppm) δ 10.90 (s, 1 H), 8.27 (dd, J = 6.5, 2.7 Hz, 1 H), 7.92-7.84 (m, 2 H), 7.70-7.60 (m, 2 H), 7.42 (t, J = 9.3 Hz, 1 H), 7.23 (s, 1 H), 7.06 (dd, J = 8.7, 3.4 Hz, 1 H), 4.68 (s, 1 H), 3.18 (s, 3 H), 2.33 (s, 3 H). |
| 31A | 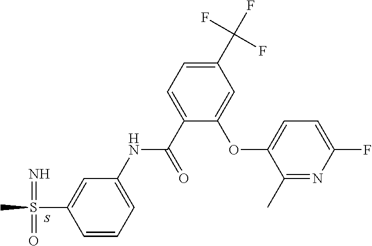 | 468.0 | ¹H NMR (400 MHz, DMSO-d₆, ppm) δ 10.90 (s, 1 H), 8.34 (s, 1 H), 7.89 (d, J = 7.8 Hz, 1 H), 7.83 (d, J = 8.1 Hz, 1 H), 7.70-7.52 (m, 4 H), 7.24 (s, 1 H), 7.06 (dd, J = 8.7, 3.4 Hz, 1 H), 4.20 (s, 1 H), 3.04 (s, 3 H), 2.33 (s, 3 H). |
| 32A | 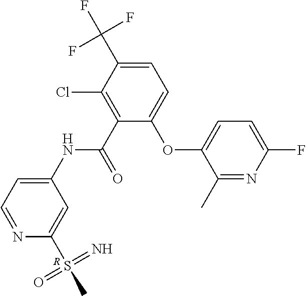 | 503.0 | ¹H NMR (400 MHz, DMSO-d₆, ppm) δ 11.69 (s, 1 H), 8.64 (d, J = 5.4 Hz, 1 H), 8.44 (d, J = 1.8 Hz, 1 H), 7.90 (d, J = 9.0 Hz, 1 H), 7.85-7.77 (m, 2 H), 7.15 (dd, J = 8.7, 3.4 Hz, 1 H), 6.93 (d, J = 8.9 Hz, 1 H), 4.40 (s, 1 H), 3.15 (s, 3 H), 2.28 (s, 3 H). |
| 33A | 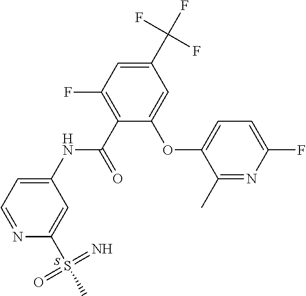 | 487.1 | ¹H NMR (400 MHz, DMSO-d₆, ppm) δ 11.75 (s, 1 H), 8.69 (d, J = 5.4 Hz, 1 H), 8.45 (d, J = 1.3 Hz, 1 H), 7.84-7.73 (m, 3 H), 7.14-7.07 (m, 2 H), 3.32 (s, 3 H), 2.30 (s, 3 H). |
| 34A | 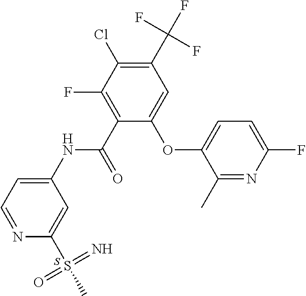 | 521.0 | ¹H NMR (400 MHz, DMSO-d₆, ppm) δ 11.72 (s, 1 H), 8.65 (d, J = 5.3 Hz, 1 H), 8.36 (d, J = 1.3 Hz, 1 H), 7.83-7.70 (m, 2 H), 7.23 (s, 1 H), 7.10 (dd, J = 8.7, 3.4 Hz, 1 H), 4.41 (s, 1 H), 3.15 (s, 3 H), 2.31 (s, 3 H). |
| 35A | 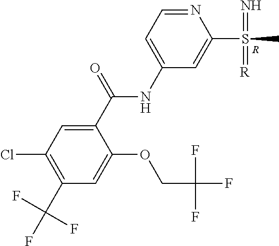 | 475.9 | ¹H NMR (400 MHz, DMSO-d₆, ppm) δ 11.18 (s, 1 H), 8.63 (d, J = 5.4 Hz, 1 H), 8.41 (d, J = 1.7 Hz, 1 H), 7.97 (s, 1 H), 7.79-7.72 (m, 2 H), 5.03 (q, J = 8.7 Hz, 2 H), 4.39 (s, 1 H), 3.15 (s, 3 H). |
| 36A | 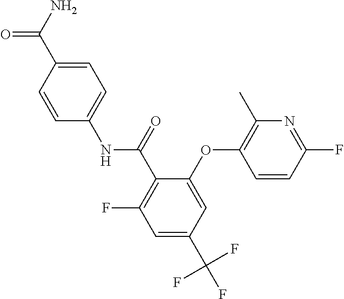 [SMILES: Cc1nc(F)ccc1Oc1cc(C(F)(F)F)cc(F)c1C(=O)Nc1ccc(C(N)=O)cc1] | 452.0 | ¹H NMR (400 MHz, DMSO-d₆, ppm) δ 11.06 (s, 1 H), 7.88 (d, J = 8.7 Hz, 3 H), 7.79-7.65 (m, 4 H), 7.29 (s, 1 H), 7.14- 7.04 (m, 2 H), 2.31 (s, 3 H). |
| 37A | 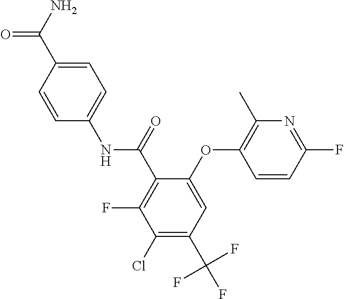 [SMILES: Cc1nc(F)ccc1Oc1cc(C(F)(F)F)c(Cl)c(F)c1C(=O)Nc1ccc(C(N)=O)cc1] | 486.0 | ¹H NMR (400 MHz, DMSO-d₆, ppm) δ 11.11 (s, 1 H), 7.96- 7.83 (m, 3 H), 7.76 (dd, J = 8.6, 6.8 Hz, 1 H), 7.66 (d, J = 8.6 Hz, 2 H), 7.35-7.19 (m, 2 H), 7.08 (dd, J = 8.7, 3.4 Hz, 1 H), 2.31 (s, 3 H). |
| 38A | 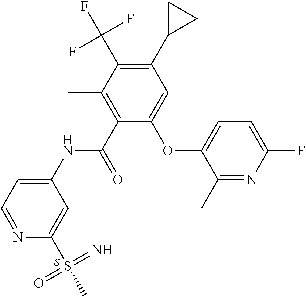 | 523.2 | ¹H NMR (400 MHz, DMSO-d₆, ppm) δ 11.44 (s, 1 H), 8.60 (d, J = 5.4 Hz, 1 H), 8.44 (d, J = 1.7 Hz, 1 H), 7.75 (dd, J = 5.4, 2.0 Hz, 1 H), 7.69 (dd, J = 8.6, 6.9 Hz, 1 H), 7.09 (dd, J = 8.7, 3.4 Hz, 1 H), 6.43 (s, 1 H), 4.36 (s, 1 H), 3.14 (s, 3 H), 2.45- 2.41 (m, 3 H), 2.21- 2.15 (m, 1 H), 0.97- 0.90 (m, 2 H), 0.58- 0.52 (m, 2 H). |
| 39A | 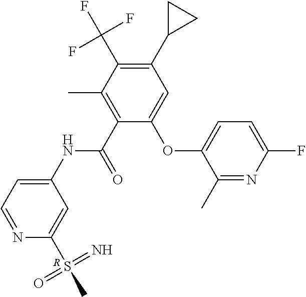 | 523.1 | ¹H NMR (400 MHz, DMSO-d₆, ppm) δ 11.45 (s, 1 H), 8.61 (d, J = 5.4 Hz, 1 H), 8.44 (d, J = 1.8 Hz, 1 H), 7.75 (dd, J = 5.4, 1.8 Hz, 1 H), 7.70 (dd, J = 8.6, 6.9 Hz, 1 H), 6.43 (s, 1 H), 4.37 (s, 1 H), 3.14 (s, 3 H), 2.47-2.37 (m, 3 H), 2.26 (s, 3 H), 2.21-2.15 (m, 1 H), 0.99-0.84 (m, 2 H), 0.58-0.52 (m, 2 H). |
| 40A | 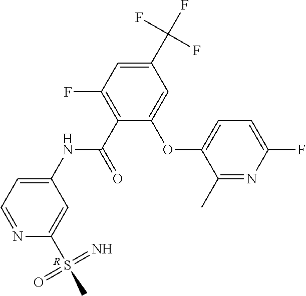 | 487.0 | ¹H NMR (400 MHz, DMSO-d₆, ppm) δ 11.83 (s, 1 H), 8.72 (d, J = 5.3 Hz, 1 H), 8.49 (s, 1 H), 7.83 (d, J = 3.9 Hz, 3 H), 7.81-7.73 (m, 2 H), 7.15-7.05(m, 2 H), 4.63 (s, 1 H), 3.44 (s, 3 H), 2.31 (s, 3 H). |
| 41A | 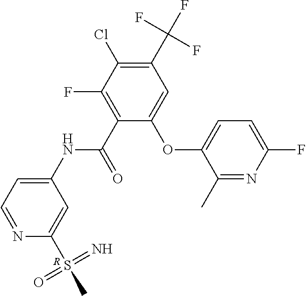 | 521.0 | ¹H NMR (400 MHz, DMSO-d₆, ppm) δ 11.83 (s, 1 H), 8.71 (d, J = 5.4 Hz, 1 H), 8.43 (d, J = 1.5 Hz, 1 H), 7.83-7.75 (m, 2 H), 7.23 (s, 1 H), 7.10 (dd, J = 8.7, 3.4 Hz, 1 H), 3.35 (s, 3 H), 2.31 (s, 3 H). |
| 42A | 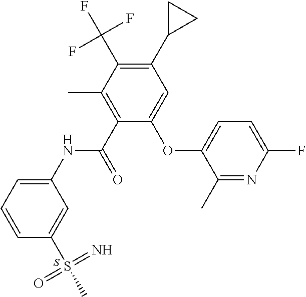 | 522.1 | ¹H NMR (400 MHz, DMSO-d₆, ppm) δ 11.00 (s, 1 H), 8.38 (s, 1 H), 7.82 (d, J = 8.1 Hz, 1 H), 7.74- 7.62 (m, 2 H), 7.57 (t, J = 7.9 Hz, 1 H), 7.08 (dd, J = 8.7, 3.4 Hz, 1 H), 6.44 (s, 1 H), 4.20 (s, 1 H), 3.05 (s, 3 H), 2.47-2.42 (m, 3 H), 2.27 (s, 3 H), 2.23-2.13 (m, 1 H), 0.99-0.84 (m, 2 H), 0.58-0.52 (m, 2 H). |
| 43A | 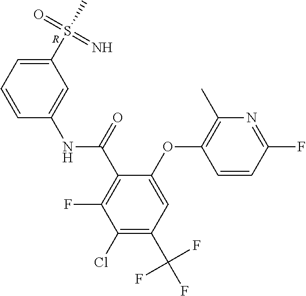 | 519.9 | ¹H NMR (400 MHz, DMSO-d₆, ppm) δ 11.28 (s, 1 H), 8.28 (t, J = 1.7 Hz, 1 H), 7.84-7.68 (m, 3 H), 7.61 (t, J = 7.9 Hz, 1 H), 7.23 (s, 1 H), 7.09 (dd, J = 8.7, 3.4 Hz, 1 H), 4.26 (s, 1 H), 3.06 (s, 3 H), 2.32 (s, 3 H). |
| 44A | 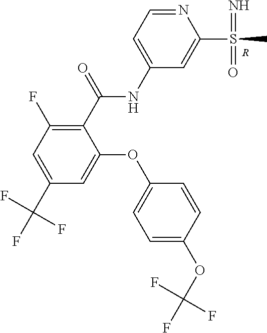 | 538.1 | ¹H NMR (400 MHz, DMSO-d₆, ppm) δ 11.63 (s, 1 H), 8.62 (d, J = 5.4 Hz, 1 H), 8.34 (d, J = 1.7 Hz, 1 H), 7.80 (d, J = 8.7 Hz, 1 H), 7.75 (dd, J = 5.4, 1.9 Hz, 1 H), 7.45 (d, J = 8.7 Hz, 2 H), 7.32-7.25 (m, 2 H), 7.22 (s, 1 H), 4.38 (s, 1 H), 3.13 (s, 3 H). |
| 45A | 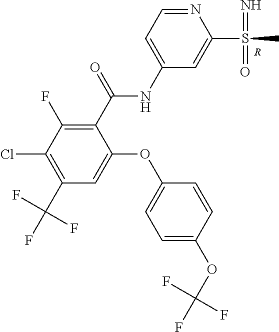 | 571.8 | ¹H NMR (400 MHz, DMSO-d₆, ppm) δ 11.67 (s, 1 H), 8.63 (d, J = 5.4 Hz, 1 H), 8.32 (d, J = 1.8 Hz, 1 H), 7.72 (dd, J = 5.4, 2.0 Hz, 1 H), 7.46- 7.39 (m, 3 H), 7.3- 7.27 (m, 2 H), 4.39 (s, 1 H), 3.14 (s, 3 H). |
| 46A | 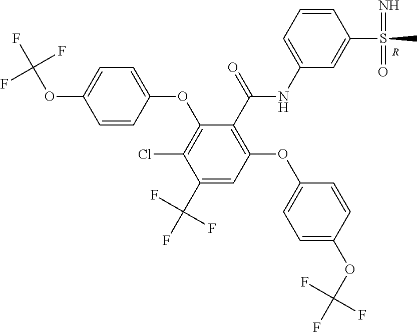 | 729.0 | ¹H NMR (400 MHz, DMSO-d₆, ppm) δ 11.08 (s, 1 H), 8.09 (s, 1 H), 7.63 (d, J = 8.3 Hz, 2 H), 7.54- 7.42 (m, 4 H), 7.34 (dd, J = 8.8, 4.4 Hz, 4 H), 7.11-7.05 (m, 2 H), 4.18 (s, 1 H), 3.01 (s, 3 H). |
| 47A | 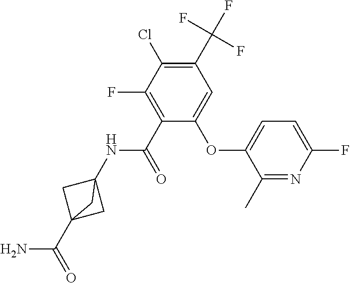 [SMILES: Cc1nc(F)ccc1Oc1cc(C(F)(F)F)c(Cl)c(F)c1C(=O)NC12CC(C(N)=O)(C1)C2] | 476 | ¹H NMR (400 MHz, DMSO-d₆, ppm) δ 9.35 (s, 1 H), 7.64 (dd, J = 8.7, 6.7 Hz, 1 H), 7.27 (s, 2 H), 7.05 (dd, J = 8.7, 3.4 Hz, 1 H), 6.99 (s, 1 H), 2.35 (s, 3 H), 2.12 (s, 6 H). |
| 48A | 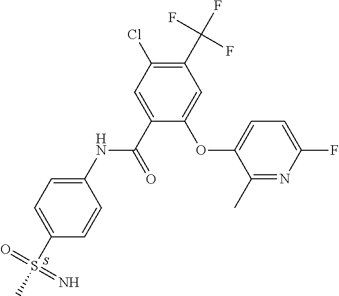 | 501.9 | ¹H NMR (400 MHz, DMSO-d₆, ppm) δ 10.99 (s, 1 H), 8.11 (s, 1 H), 7.90 (d, J = 8.8 Hz, 2 H), 7.81 (d, J = 8.8 Hz, 2 H), 7.67 (dd, J = 8.6, 6.8 Hz, 1 H), 7.39 (s, 1 H), 7.04 (dd, J = 8.7, 3.4 Hz, 1 H), 4.14 (s, 1 H), 3.03 (d, J = 0.7 Hz, 3 H), 2.33 (s, 3 H). |
| 49A | 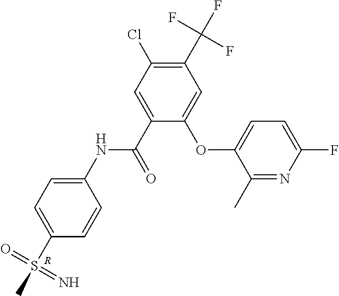 | 502 | ¹H NMR (400 MHz, DMSO-d₆, ppm) δ 11.00 (s, 1 H), 8.11 (s, 1 H), 7.91 (d, J = 8.8 Hz, 2 H), 7.85- 7.78 (d, J = 8.8 Hz, 2 H), 7.67 (dd, J = 8.6, 6.8 Hz, 1 H), 7.39 (s, 1 H), 7.04 (dd, J = 8.7, 3.4 Hz, 1 H), 4.15 (s, 1 H), 3.04 (s, 3 H), 2.34 (s, 3 H). |
| 50A | 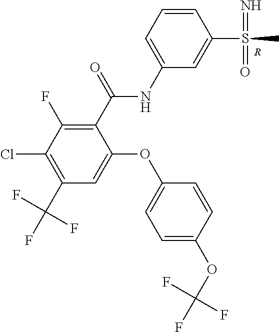 | 571 | ¹H NMR (400 MHz, DMSO-d₆, ppm) δ 11.24 (s, 1 H), 8.25 (s, 1 H), 7.79 (d, J = 7.9 Hz, 1 H), 7.68 (s, 1 H), 7.59 (d, J = 8.0 Hz, 1 H), 7.43 (d, J = 8.8 Hz, 2 H), 7.37 (s, 1 H), 7.28 (d, J = 9.1 Hz, 2 H), 4.22 (s, 1 H), 3.05 (s, 3 H). |
| 51A | 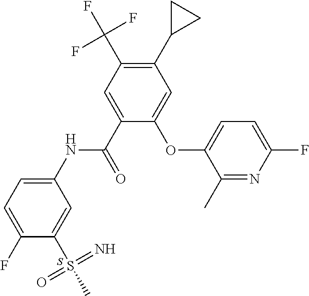 | 526.1 | ¹H NMR (400 MHz, DMSO-d₆, ppm) δ 10.74 (s, 1 H), 8.22 (dd, J = 6.5, 2.5 Hz, 1 H), 7.95 (s, 1 H), 7.87 (d, J = 9.1 Hz, 1 H), 7.64-7.53 (m, 1 H), 7.41 (t, J = 9.4 Hz, 1 H), 7.03 (dd, J = 8.7, 3.6 Hz, 1 H), 6.55 (s, 1 H), 4.66 (s, 1 H), 3.17 (s, 3 H), 2.32 (s, 3 H), 2.14 (s, 1 H), 1.09-0.99 (m, 2 H), 0.71 (d, J = 5.6 Hz, 2 H). |
| 52A | 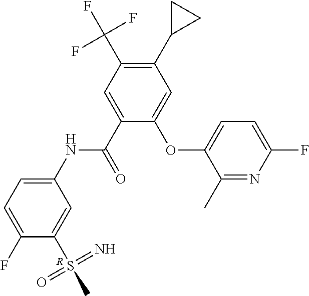 | 526.1 | ¹H NMR (400 MHz, DMSO-d₆, ppm) δ 10.75 (s, 1 H), 8.22 (dd, J = 6.4, 2.6 Hz, 1 H), 7.95 (s, 1 H), 7.89-7.84 (m, 1 H), 7.59 (dd, J = 8.6, 6.9 Hz, 1 H), 7.41 (t, J = 9.4 Hz, 1 H), 7.03 (dd, J = 8.7, 3.4 Hz, 1 H), 6.55 (s, 1 H), 4.67 (s, 1 H), 3.17 (s, 3 H), 2.32 (s, 3 H), 2.14 (s, 1 H), 1.05 (dd, J = 8.3, 2.0 Hz, 2 H), 0.71 (d, J = 6.2 Hz, 2 H). |
| 53A | 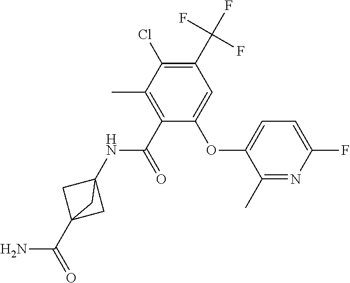 [SMILES: Cc1nc(F)ccc1Oc1cc(C(F)(F)F)c(Cl)c(C)c1C(=O)NC12CC(C(N)=O)(C1)C2] | 472.0 | ¹H NMR (400 MHz, DMSO-d₆, ppm) δ 9.15 (s, 1 H), 7.55 (dd, J = 8.6, 6.8 Hz, 1 H), 7.28 (s, 1 H), 7.21 (s, 1 H), 7.07- 6.94 (m, 2 H), 2.35 (d, J = 1.7 Hz, 6 H), 2.13 (s, 6 H). |
| 54A | 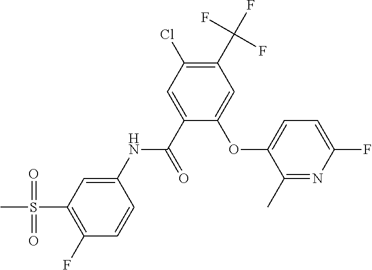 [SMILES: Cc1nc(F)ccc1Oc1cc(C(F)(F)F)c(Cl)cc1C(=O)Nc1ccc(F)c(S(C)(=O)=O)c1] | 521.0 | ¹H NMR (400 MHz, DMSO-d₆, ppm) δ 11.02 (s, 1 H), 8.30- 8.24 (m, 1 H), 8.12 (d, J = 3.2 Hz, 1 H), 7.93 (dd, J = 8.9, 3.0 Hz, 1 H), 7.69-7.62 (m, 1 H), 7.57-7.52 (m, 1 H), 7.38 (d, J = 3.5 Hz, 1 H), 7.04 (dd, J = 8.4, 3.5 Hz, 1 H), 2.50-2.49 (m, 3 H), 2.34 (d, J = 3.3 Hz, 3 H). |
| 55A | 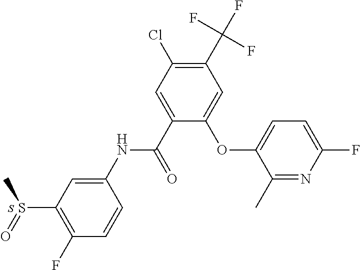 | 505.0 | ¹H NMR (400 MHz, DMSO-d₆, ppm) δ 10.96 (s, 1 H), 8.09 (dd, J = 7.1, 3.7 Hz, 2 H), 7.82-7.73 (m, 1 H), 7.66 (dd, J = 8.6, 6.8 Hz, 1 H), 7.43- 7.33 (m, 2 H), 7.04 (dd, J = 8.7, 3.5 Hz, 1 H), 2.82 (s, 3 H), 2.34 (s, 3 H). |
| 56A | 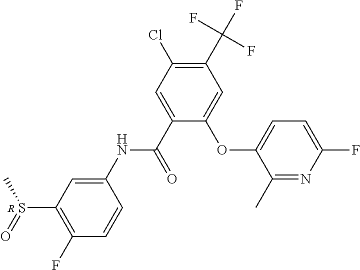 | 505.0 | ¹H NMR (400 MHz, DMSO-d₆, ppm) δ 10.96 (s, 1 H), 8.09 (dd, J = 7.1, 3.7 Hz, 2 H), 7.77 (dd, J = 5.9, 3.0 Hz, 1 H), 7.66 (dd, J = 8.7, 6.8 Hz, 1 H), 7.42-7.35 (m, 2 H), 7.04 (dd, J = 8.7, 3.4 Hz, 1 H), 2.82 (s, 3 H), 2.34 (s, 3 H). |
| 57A | 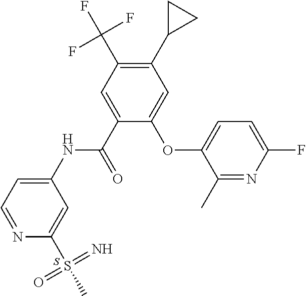 | 509.1 | ¹H NMR (400 MHz, DMSO-d₆, ppm) δ 11.17 (s, 1 H), 8.58 (d, J = 5.4 Hz, 1 H), 8.35 (s, 1 H), 8.00 (s, 1 H), 7.77 (d, J = 3.9 Hz, 1 H), 7.63-7.58 (m, 1 H), 7.03 (dd, J = 8.7, 3.4 Hz, 1 H), 6.55 (s, 1 H), 4.34 (s, 1 H), 3.13 (s, 3 H), 2.31 (s, 3 H), 2.14 (s, 1 H), 1.06 (dd, J = 8.2, 1.9 Hz, 2 H), 0.73 (t, J = 5.5 Hz, 2 H). |
| 58A | 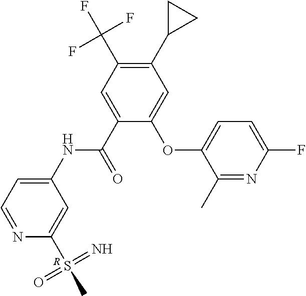 | 509.1 | ¹H NMR (400 MHz, DMSO-d₆, ppm) δ 11.17 (s, 1 H), 8.58 (d, J = 5.3 Hz, 1 H), 8.36 (s, 1 H), 8.00 (s, 1 H), 7.77 (d, J = 3.1 Hz, 1 H), 7.61 (t, J = 7.7 Hz, 1 H), 7.07- 6.99 (m, 1 H), 6.55 (s, 1 H), 4.34 (s, 1 H), 3.13 (s, 3 H), 2.31 (s, 3 H), 2.14 (s, 1 H), 1.08-1.01 (m, 2 H), 0.72 (d, J = 5.0 Hz, 2 H). |
| 59A | 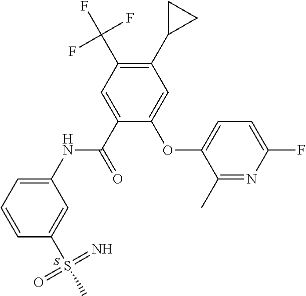 | 508.1 | ¹H NMR (400 MHz, DMSO-d₆, ppm) δ 10.76 (s, 1 H), 8.29 (s, 1 H), 7.96 (s, 1 H), 7.83 (d, J = 8.2 Hz, 1 H), 7.71-7.49 (m, 3 H), 7.03 (dd, J = 8.7, 3.4 Hz, 1 H), 6.56 (s, 1 H), 4.20 (s, 1 H), 3.04 (s, 3 H), 2.32 (s, 3 H), 2.20- 2.09 (m, 1 H), 1.13- 0.97 (m, 2 H), 0.81- 0.61 (m, 2 H). |
| 60A | 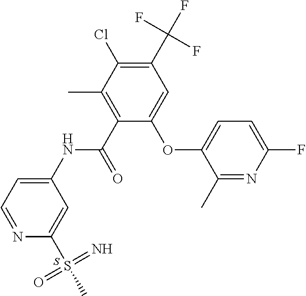 | 517.0 | ¹H NMR (400 MHz, DMSO-d₆, ppm) δ 11.54 (s, 1 H), 8.62 (d, J = 5.2 Hz, 1 H), 8.40 (s, 1 H), 7.76- 7.69 (m, 2 H), 7.16 (s, 1 H), 7.08 (dd, J = 8.7, 3.3 Hz, 1 H), 4.39 (s, 1 H), 3.14 (s, 3 H), 2.44 (s, 3 H), 2.29 (s, 3 H). |
| 61A | 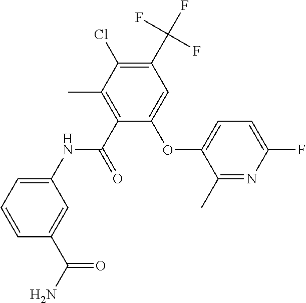 [SMILES: Cc1nc(F)ccc1Oc1cc(C(F)(F)F)c(Cl)c(C)c1C(=O)Nc1cccc(C(N)=O)c1] | 482.0 | ¹H NMR (400 MHz, DMSO-d₆, ppm) δ 10.84 (s, 1 H), 8.14 (s, 1 H), 7.97 (s, 1 H), 7.77-7.65 (m, 2 H), 7.61 (d, J = 7.8 Hz, 1 H), 7.42 (t, J = 7.9 Hz, 1 H), 7.37 (s, 1 H), 7.16 (s, 1 H), 7.06 (dd, J = 8.7, 3.4 Hz, 1 H), 2.45 (s, 3 H), 2.31 (s, 3 H). |
| 62A | 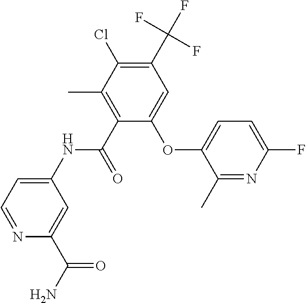 [SMILES: Cc1nc(F)ccc1Oc1cc(C(F)(F)F)c(Cl)c(C)c1C(=O)Nc1ccnc(C(N)=O)c1] | 483.0 | ¹H NMR (400 MHz, DMSO-d₆, ppm) δ 11.34 (s, 1 H), 8.55 (d, J = 5.4 Hz, 1 H), 8.30 (d, J = 1.9 Hz, 1 H), 8.11 (d, J = 1.9 Hz, 1 H), 7.78 (dd, J = 5.5, 2.1 Hz, 1 H), 7.74-7.66 (m, 2 H), 7.18 (s, 1 H), 7.07 (dd, J = 8.7, 3.4 Hz, 1 H), 2.44 (s, 3 H), 2.28 (s, 3 H). |
| 63A | 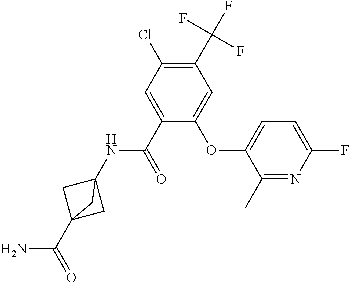 [SMILES: Cc1nc(F)ccc1Oc1cc(C(F)(F)F)c(Cl)cc1C(=O)NC12CC(C(N)=O)(C1)C2] | 458.0 | ¹H NMR (400 MHz, DMSO-d₆, ppm) δ 9.10 (s, 1 H), 7.86 (s, 1 H), 7.50 (dd, J = 8.7, 6.7 Hz, 1 H), 7.42 (s, 1 H), 7.28 (s, 1 H), 7.03-6.95 (m, 2 H), 2.38 (s, 3 H), 2.09 (s, 6 H). |
| 64A | 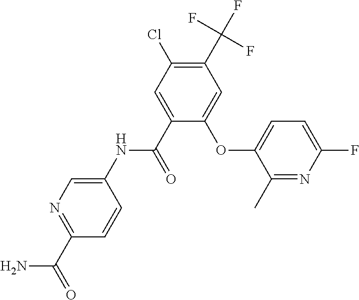 [SMILES: Cc1nc(F)ccc1Oc1cc(C(F)(F)F)c(Cl)cc1C(=O)Nc1ccc(C(N)=O)nc1] | 469.0 | ¹H NMR (400 MHz, DMSO-d₆, ppm) δ 11.11 (s, 1 H), 8.81 (d, J = 2.2 Hz, 1 H), 8.22 (dd, J = 8.6, 2.4 Hz, 1 H), 8.15 (s, 1 H), 8.04 (d, J = 8.6 Hz, 2 H), 7.67 (dd, J = 8.7, 6.8 Hz, 1 H), 7.57 (s, 1 H), 7.41 (s, 1 H), 7.04 (dd, J = 8.7, 3.4 Hz, 1 H), 2.36 (s, 3 H). |
| 65A | 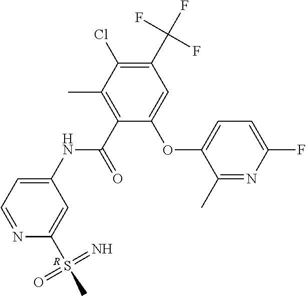 | 517.0 | ¹H NMR (400 MHz, DMSO-d₆, ppm) δ 11.52 (s, 1 H), 8.63 (d, J = 5.4 Hz, 1 H), 8.40 (d, J = 1.9 Hz, 1 H), 7.76-7.69 (m, 2 H), 7.16 (s, 1 H), 7.08 (dd, J = 8.7, 3.4 Hz, 1 H), 4.38 (s, 1 H), 3.14 (s, 3 H), 2.44 (s, 3 H), 2.29 (s, 3 H). |
| 66A | 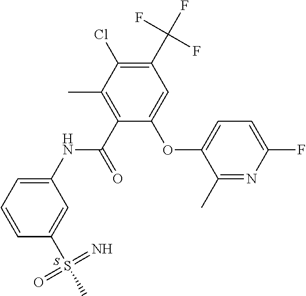 | 516.0 | ¹H NMR (400 MHz, DMSO-d₆, ppm) δ 11.07 (s, 1 H), 8.32 (d, J = 1.8 Hz, 1 H), 7.81 (d, J = 8.9 Hz, 1 H), 7.73-7.57 (m, 3 H), 7.16 (s, 1 H), 7.07 (dd, J = 8.8, 3.4 Hz, 1 H), 4.22 (s, 1 H), 3.05 (s, 3 H), 2.45 (s, 3 H), 2.30 (s, 3 H). |
| 67A | 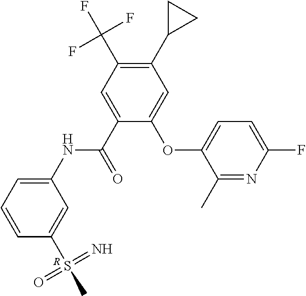 | 508.0 | ¹H NMR (400 MHz, DMSO-d₆, ppm) δ 10.74 (s, 1 H), 8.29 (s, 1 H), 7.96 (s, 1 H), 7.83 (d, J = 8.2 Hz, 1 H), 7.68-7.52 (m, 3 H), 7.03 (dd, J = 8.7, 3.4 Hz, 1 H), 6.56 (s, 1 H), 4.18 (s, 1 H), 3.04 (s, 3 H), 2.32 (s, 3 H), 2.19- 2.07 (m, 1 H), 1.09- 1.00 (m, 2 H), 0.75- 0.68 (m, 2 H). |
| 68A | 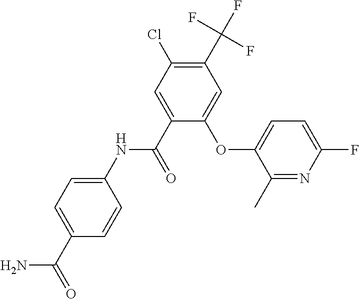 [SMILES: Cc1nc(F)ccc1Oc1cc(C(F)(F)F)c(Cl)cc1C(=O)Nc1ccc(C(N)=O)cc1] | 467.9 | ¹H NMR (400 MHz, DMSO-d₆, ppm) δ 10.83 (s, 1 H), 8.09 (s, 1 H), 7.95-7.82 (m, 3 H), 7.66 (t, J = 9.1 Hz, 3 H), 7.39 (s, 1 H), 7.29 (s, 1 H), 7.03 (dd, J = 8.6, 3.1 Hz, 1 H), 2.33 (s, 3 H). |
| 69A | 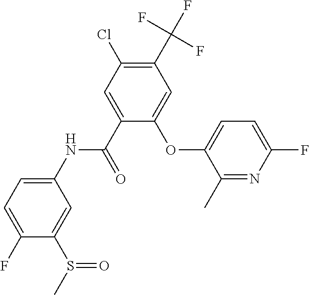 [SMILES: Cc1nc(F)ccc1Oc1cc(C(F)(F)F)c(Cl)cc1C(=O)Nc1ccc(F)c(S(C)=O)c1] | 504.9 | ¹H NMR (400 MHz, DMSO-d₆, ppm) δ 10.96 (s, 1 H), 8.09 (dd, J = 7.1, 3.7 Hz, 2 H), 7.8-7.74 (m, 1 H), 7.66 (dd, J = 8.7, 6.8 Hz, 1 H), 7.43- 7.34 (m, 2 H), 7.04 (dd, J = 8.7, 3.4 Hz, 1 H), 2.82 (s, 3 H), 2.34 (s, 3 H). |
| 70A | 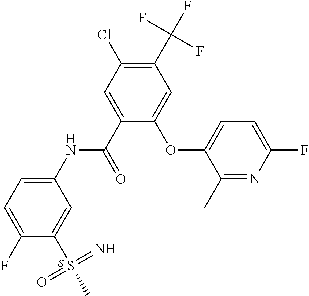 | 519.9 | ¹H NMR (400 MHz, DMSO-d₆, ppm) δ 10.97 (s, 1 H), 8.23 (dd, J = 6.3, 2.4 Hz, 1 H), 8.10 (s, 1 H), 7.87 (dd, J = 8.2, 3.8 Hz, 1 H), 7.71-7.63 (m, 1 H), 7.43 (t, J = 9.3 Hz, 1 H), 7.36 (s, 1 H), 7.05 (dd, J = 8.7, 3.3 Hz, 1 H), 4.70 (s, 1 H), 3.19 (s, 3 H), 2.34 (s, 3 H). |
| 71A | 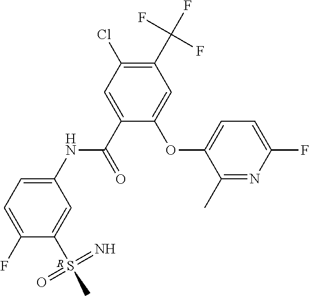 | 520.0 | ¹H NMR (400 MHz, DMSO-d₆, ppm) δ 10.97 (s, 1 H), 8.29- 8.19 (m, 1 H), 8.10 (d, J = 3.8 Hz, 1 H), 7.87 (dd, J = 8.1, 3.6 Hz, 1 H), 7.67 (dd, J = 9.7, 5.5 Hz, 1 H), 7.48-7.40 (m, 1 H), 7.37 (d, J = 3.9 Hz, 1 H), 7.05 (dd, J = 8.4, 3.7 Hz, 1 H), 4.70 (s, 1 H), 3.19 (s, 3 H), 2.34 (s, 3 H). |
| 72A | 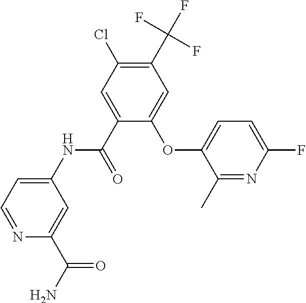 [SMILES: Cc1nc(F)ccc1Oc1cc(C(F)(F)F)c(Cl)cc1C(=O)Nc1ccnc(C(N)=O)c1] | 469.0 | ¹H NMR (400 MHz, DMSO-d₆, ppm) δ 11.19 (s, 1 H), 8.54 (d, J = 5.5 Hz, 1 H), 8.28 (d, J = 1.8 Hz, 1 H), 8.15 (s, 1 H), 8.10 (s, 1 H), 7.79 (dd, J = 5.4, 2.0 Hz, 1 H), 7.69-7.66 (m, 2 H), 7.38 (s, 1 H), 7.04 (dd, J = 8.7, 3.4 Hz, 1 H), 2.33 (s, 3 H). |
| 73A | 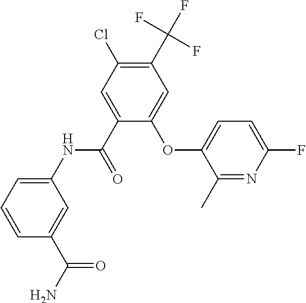 [SMILES: Cc1nc(F)ccc1Oc1cc(C(F)(F)F)c(Cl)cc1C(=O)Nc1cccc(C(N)=O)c1] | 468.0 | ¹H NMR (400 MHz, DMSO-d₆, ppm) δ 10.74 (s, 1 H), 8.12 (s, 1 H), 8.08 (s, 1 H), 7.96 (s, 1 H), 7.79-7.72 (m, 1 H), 7.67 (dd, J = 8.6, 6.8 Hz, 1 H), 7.61 (d, J = 7.7 Hz, 1 H), 7.44- 7.35 (m, 3 H), 7.04 (dd, J = 8.7, 3.5 Hz, 1 H), 2.34 (s, 3 H). |

### Example 74A

### Compound Profiling Human NaV1.8/β3 Cell Line—SyncroPatch384PE Assay

Compounds were tested on recombinant human Nav1.8/b3 stably transfected CHO cells using the SyncroPatch384PE system (Nanion Technologies), an automated patch clamp device. Cells were cultured at 37° C./5% CO₂ in Ham's F-12 supplemented with 10% fetal bovine serum, 100 U/mL penicillin G sodium, 100 mg/mL streptomycin sulfate and selection antibiotics (0.01 mg/ml Blasticidin, 0.4 mg/ml Zeocin and 0.25 mg/ml Hygromycin). On the day of the recordings, cells in culture dishes were washed twice with Hank's Balanced Salt Solution (HBSS) and treated with Accutase for approximately 20 minutes. Immediately before use in the SynchroPatch384PE using an 8-hole NPC-384 chip, the cells were washed in HBSS to remove the Accutase and resuspended in extracellular solution. All experiments were performed at ambient temperature. Intracellular solution contained (mM): CsCl, 50; CsF, 90; MgCl₂, 5; EGTA, 1; HEPES, 10; pH adjusted to 7.2 with CsOH. Extracellular solution contained (mM): NaCl, 137; KCl, 4.0; CaCl₂), 3.8; MgCl₂, 1; HEPES, 10; Glucose, 10; pH adjusted to 7.4 with NaOH. 100 nM tetrodotoxin (TTX) was added to the extracellular solution to block endogenous TTX-sensitive sodium currents.

Compounds were tested in quadruplicate in 0.3% DMSO and 0.03% pluronic Acid. Compounds were diluted 1:3.33 in extracellular solution to create an 8-point concentration response curve. Each plate contained a historical positive control and up to ten compounds. 300 μM tetracaine and 0.3% DMSO+0.03% pluronic acid were used as high and low controls respectively.

Whole cell patch clamp recordings were conducted according to Nanion's standard procedure for SyncroPatch384PE®. Cells were held at a holding potential of −120 mV. A depolarization step to 10 mV for 30 ms was applied (P1 measurement), followed by a hyperpolarization step to −100 mV for 100 ms. An inactivation step at −35 mV for 10 sec was applied before stepping to −100 mV for 20 ms, followed by a step to 10 mV for 30 ms (P2 measurement) and then back to −100 mV for 30 ms. Sweep interval was 15 sec. Following establishment of the whole-cell configuration in extracellular solution, cells were washed in extracellular solution containing 0.3% DMSO and 0.03% pluronic acid to stabilize the baseline current. Compounds were then applied by the SynchroPatch384 PE system into each well and the current was recorded for five minutes in extracellular solution, followed by application of tetracaine to achieve full block at the end of experiment. The potency of the compounds was assessed on two read-outs, resting state block (P1 measurement) or inactivated state block (P2 measurement) to obtain IC50 values. Values were normalized to high (tetracaine) and low (DMSO+pluronic acid) controls.

The table below shows the potency of compounds against human Nav1.8, where “A” represents an IC₅₀ less than or equal to 5 nM, “B” represents an IC₅₀ greater than 5 nM to less than or equal to 50 nM, “C” represents an IC₅₀ greater than 50 nM to less than or equal to 100 nM, “D” represents an IC₅₀ greater than 100 nM to less than or equal to 200 nM, “E” represents an IC50 greater than 200 nM.

| Example | P1 | P2 |
|---|---|---|
| 1A | A | A |
| 2A | B | B |
| 3A | A | A |
| 4A | A | A |
| 5A | B | B |
| 6A | A | A |
| 7A | E | E |
| 9A | A | A |
| 10A | A | A |
| 13A | A | A |
| 14A | A | A |
| 33A | B | B |
| 34A | A | A |
| 35A | C | C |
| 36A | B | B |
| 37A | A | A |
| 38A | A | A |
| 39A | A | A |
| 40A | B | B |
| 41A | A | A |
| 42A | A | A |
| 43A | A | A |
| 44A | A | A |
| 45A | A | A |
| 46A | D | D |
| 47A | E | E |
| 48A | C | C |
| 49A | C | C |
| 50A | A | A |
| 51A | A | A |
| 52A | A | A |
| 53A | E | E |
| 54A | A | A |
| 55A | A | A |
| 56A | A | A |
| 57A | A | B |
| 58A | A | A |
| 59A | A | A |
| 60A | A | A |
| 61A | A | A |
| 62A | A | A |
| 63A | E | E |
| 64A | B | B |
| 65A | A | A |
| 66A | A | A |
| 67A | A | A |
| 68A | B | A |
| 69A | A | A |
| 70A | A | A |
| 71A | A | A |
| 72A | A | A |
| 73A | A | A |

### Example 75A

### Compound Profiling Human NaV1.8/β1 Cell Line—Sophion QPatch II Assay

Compounds were tested on recombinant human Nav1.8/β1 stably transfected HEK293 (Eurofins, CYL3025, St. Charles, MO) cells using the QPatch II system (Sophion Bioscience A/S, Ballerup—Denmark), an automated patch clamp device. Cells were cultured at 37° C./5% CO₂ in DMEM/F-12 supplemented with 10% fetal bovine serum, 1× Non-Essential Amino Acids, and selection antibiotics (0.625 μg/mL Puromycin, 400 μg/mL Geneticin, and 100 μg/mL Hygromycin). One or two days prior to recording, cells were moved to a lower temperature 30° C./5% CO₂ incubator to increase surface expression of the channels. On the day of the recordings, cells in culture dishes were washed twice with Ca²⁺—Mg²⁺ free DPBS and treated with Detachin (Genlantis, San Diego) for 3 minutes and then resuspended in Serum Free Medium (EX-CELL® ACF CHO Medium for Cell Culture (Sigma-Aldrich) supplemented with 0.04 mg/mL soybean trypsin Inhibitor and 25 mM HEPES (all components supplied by Sigma-Aldrich, St. Louis, MO) and allowed to recover for a minimum of 20 minutes in the onboard QStirrer. All experiments were performed at ambient temperature. Intracellular solution contained (mM): CsCl, 50; CsF, 90; MgCl₂, 2; EGTA, 5; HEPES, 10; pH adjusted to 7.2 with CsOH. Extracellular solution contained (mM): NaCl, 137; KCl, 4; CaCl₂), 3.8; MgCl₂, 1; HEPES, 10; Glucose, 10; pH adjusted to 7.4 with NaOH. 500 nM tetrodotoxin (TTX) was added to the extracellular solution to block endogenous TTX-sensitive sodium currents.

Compounds were tested in an interleaved manner across two cohorts of wells to develop an aggregate 8-point concentration response curve. Final concentrations include 0.1% DMSO and 0.03% pluronic Acid. In general, the concentration range spans a range from 0.01-300 nM. Compounds were diluted 1:10 with extracellular solution within their respective cohort. DMSO (0.1% by volume) served as a vehicle control to ascertain assay performance.

Whole cell patch clamp recordings were conducted according to Sophion standard procedure for QPatch II®. Cells were held at a holding potential of −120 mV. A depolarizing step to 10 mV for 30 ms was applied to elicit inward Na⁺ currents. Inter-sweep interval was 15 sec. Following establishment of the whole-cell configuration in extracellular solution, cells were washed twice in extracellular solution containing 0.1% DMSO and 0.03% pluronic acid to stabilize the baseline current. Compounds were then applied by the QPatch II system into each well in an ascending manner within cohort and the current was recorded for five minutes for each concentration. Percent of current remaining was determined by taking the average of the last 5 sweeps within each concentration and normalizing to the latest pre-compound saline period. The potency (IC₅₀) values of the compounds were only assessed against resting state block. Wells whose initial sodium current magnitude were below 2 nA or whole cell resistance fell below 100 MΩ were excluded from analysis.

The table below shows the potency of compounds against human Nav1.8, where “A” represents an IC₅₀ less than or equal to 5 nM, “B” represents an IC₅₀ greater than 5 nM to less than or equal to 50 nM, “C” represents an IC₅₀ greater than 50 nM to less than or equal to 100 nM, “D” represents an IC₅₀ greater than 100 nM to less than or equal to 200 nM, “E” represents an IC₅₀ greater than 200 nM.

| Example | P1 |
|---|---|
| 8A | B |
| 11A | A |
| 12A | B |
| 15A | B |
| 16A | A |
| 17A | B |
| 18A | E |
| 19A | E |
| 20A | D |
| 21A | D |
| 22A | C |
| 23A | E |
| 24A | D |
| 25A | E |
| 26A | E |
| 27A | E |
| 28A | E |
| 29A | E |
| 30A | D |
| 31A | D |
| 32A | B |

### (B) Second Set of Compounds

In one aspect, the invention provides a compound of Formula (I):

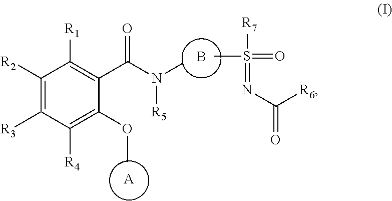

and pharmaceutically acceptable salts, hydrates, and solvates thereof, wherein:
A and B are independently aryl or heteroaryl, wherein the aryl or heteroaryl is unsubstituted or substituted with one or more groups selected from the group consisting of halo-C₁-C₄ alkyl wherein the haloalkyl chain may be fully or partially halogenated, substituted or unsubstituted C₁-C₈ alkyl, deuterated C₁-C₄ alkyl wherein the alkyl chain may be fully or partially deuterated, C₃-C₁₀ cycloalkyl, halogen, cyano, nitro, C₁-C₈ alkoxyl, aloalkoxyl wherein the haloalkyoxy chain may be fully or partially halogenated, and arylalkoxyl;
R₁, R₂, R₃ and R₄ are independently selected from hydrogen, —OH, halogen, C₁-C₆-alkyl, C₁-C₆ fluoroalkyl wherein the fluoroalkyl chain may be fully or partially fluorinated, C₃-C₈ branched alkyl, C₃-C₈ branched fluoroalkyl wherein the branched fluoroalkyl chain may be fully or partially fluorinated, alkenyl, alkynyl, haloalkyl, alkoxy, cycloalkoxy, haloalkoxy, thioalkoxy, nitro, cyano, —C(R′)(R″)-cycloalkyl, C(R′)(R′)-aryl, NR′R″, 3-8, membered cycloalkyl, 3-8-membered cycloalkenyl 3-6 membered heterocycloalkyl, any of which may have one or more substituents, wherein the 3-6 membered heterocycloalkyl comprises at least one heteroatom independently selected from O, S, and N, a saturated or unsaturated 5 or more membered ring or an aryl ring optionally containing 1 or more heteroatoms independently selected from 0, S, and N, wherein each 5 or more membered ring is unsubstituted or substituted with one to five substituents selected from hydrogen, cyano, halo, or methyl; a fused ring formed by at least two of R₁, R₂, R₃ and R₄ wherein the fused ring is selected from a group consisting of: optionally saturated carbocyclyl or heterocyclyl containing 5-6 ring members, wherein the heterocyclyl include one or more heteroatoms;
R₅ is H, or substituted or unsubstituted C₁-C₃ alkyl;
R₆ is —(CH₂)ₙRₐ, —(CR_{b}R_{c})ₙRₐ, —(CR_{b}R_{c})ₙ—(OCH₂CH₂)ₙRₐ, —(CR_{b}R_{c})ₙ—(NR′CH₂CH₂)ₙRₐ, —(CR_{b}R_{c})ₙ—(NR′CH₂CH₂)ₙRₐ, or —(CR_{b}R_{c})ₙ—(NHCH₂CH₂NH)ₙRₐ;
   wherein Rₐ is H, C₁-C₆-alkyl, C₂-C₈ branched alkyl, C₃-C₈ cycloalkyl, aryl, heteroaryl, 4-7 member heterocyclyl, alkenyl, alkynyl, haloalkyl, OH, alkoxy, cycloalkoxy, haloalkoxy, —(OCH₂CH₂)ₙR_{b}, —NR′R″, —COONR′R″, —COOR′, alkylsulfonyl, arylsulfonyl, or —SO₂NR′R″;
   R_{b} is independently selected from H, F, C₁-C₆-alkyl, haloalkyl, branched alkyl, aryl, heteroaryl, 3-7 member carbocyclyl, 4-7 member heterocyclyl with one or more heteroatoms;
   R_{c} is H, C₁-C₆ alkyl or F;
   R_{c} and R_{b} optionally form a 3-6 member carbocyclic ring or 4-6 member heterocyclic ring with one or more hetero atoms;
   R₇ is NH₂, —NHR′, C₁-C₃ alkyl, substituted or unsubstituted C₃-C₄ cycloalkyl, haloalkyl, halocycloalkyl, aryl, heteroaryl, or heterocyclyl.

In various embodiments, the compound of the invention is a compound of Formula (II):

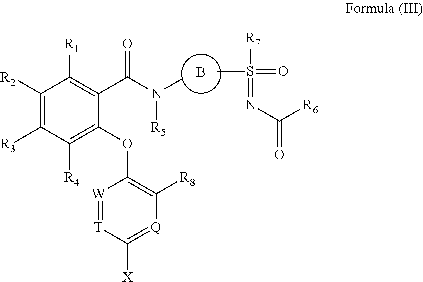

wherein ring B, and groups R₁, R₂, R₃, R₄, R₅, R₆, and R₇, are described in claim 1;
R₈ is H, hydroxyl, halogen, —CD₃, C₁-C₆-alkyl, branched alkyl, haloalkyl where the alkyl chain is fully or partially halogenated, alkoxy, arylalkoxy, cycloalkoxy, haloalkoxy, cyano, —CH₂-cycloalkyl, —CH(CH₃)-cycloalkyl, trifluoromethyl, cyclopropylmethyl, 3-6 membered cycloalkyl, or 3-6 membered heterocycloalkyl, any of which may have one or more substituents, wherein the 3-6 membered heterocycloalkyl comprises at least one heteroatom independently selected from O, S, and N;
Q, T, and W are independently selected from N or CR₉;
R₉ is H, halogen, —CD₃, alkyl, haloalkyl, alkoxy, haloalkoxy, cyano, —CF₃, —OCF₃, or cycloalkoxy, each of which is optionally substituted;
X is H, halogen, —CD₃, alkyl, haloalkyl, alkoxy, cycloalkoxy, haloalkoxy, cyano, —CF₃, or —OCF₃.

In another embodiment, the compound of the invention is a compound of Formula (III):

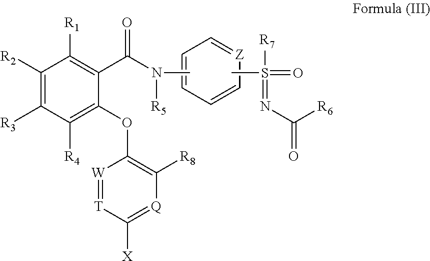

wherein, R₁, R₂, R₃, R₄, R₅, R₆, and R₇ are described above in Formula (I);
R₈, W, T, Q, and X are described above in Formula (II);
Z is CH, N, CF, or N⁺—O⁻

In another embodiment, X is F.

In another embodiment, R₈ is —CH₃.

In another embodiment, Q is N.

In another embodiment, Q is N, W is CH, and T is CH.

In another embodiment, R₁ is H or F.

In another embodiment, R₂ is H, chloro, or CF₃.

In another embodiment, R₃ is H or CF₃.

In another embodiment, R₄ is H.

In another embodiment, R₅ is H.

In another embodiment, R₇ is methyl.

In another embodiment, R₆ is azetidine, pyrrolidine, —CH₂—OH, —CH—(CH₃)—OH, —CH—CH₂—NH—CH₃, —CH₂—NH₂, CH—(CH₃)—NH₂, —CH₂—NH₂, —C—(CH₃)₂—NH₂, or -cyclobutyl-NH₂.

In another embodiment, R₉ is H.

In another embodiment, the compound of Formula (I) is selected from the group consisting of:

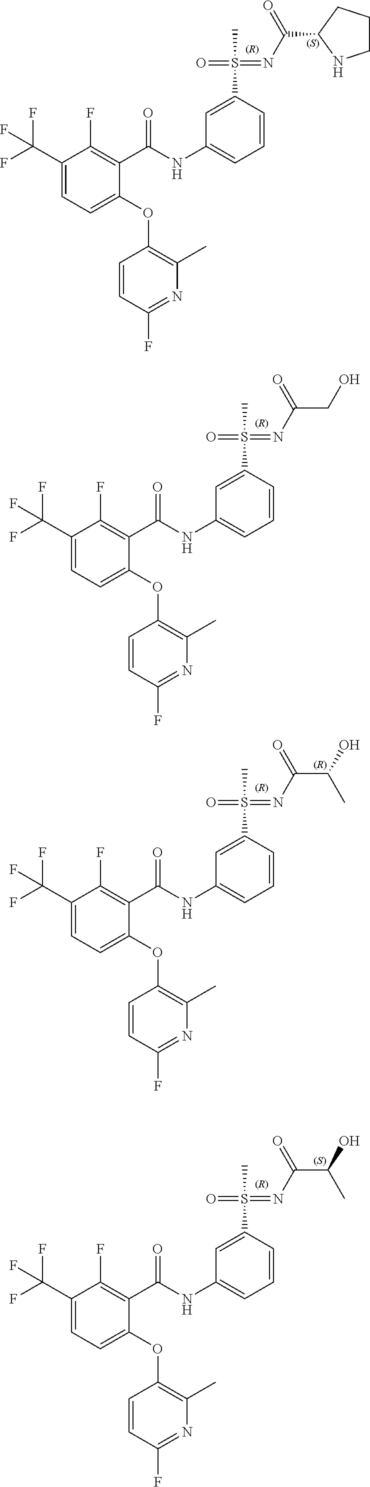

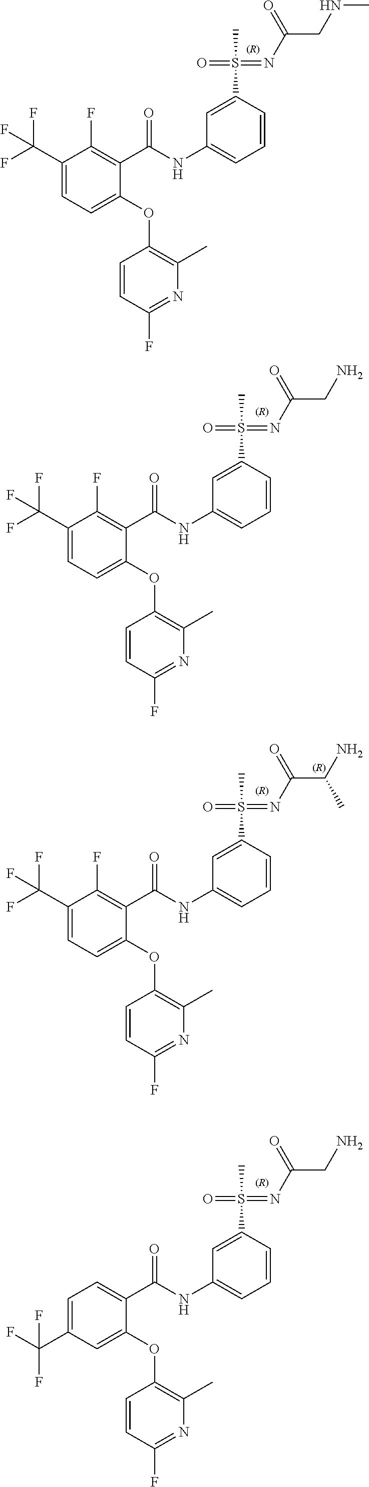

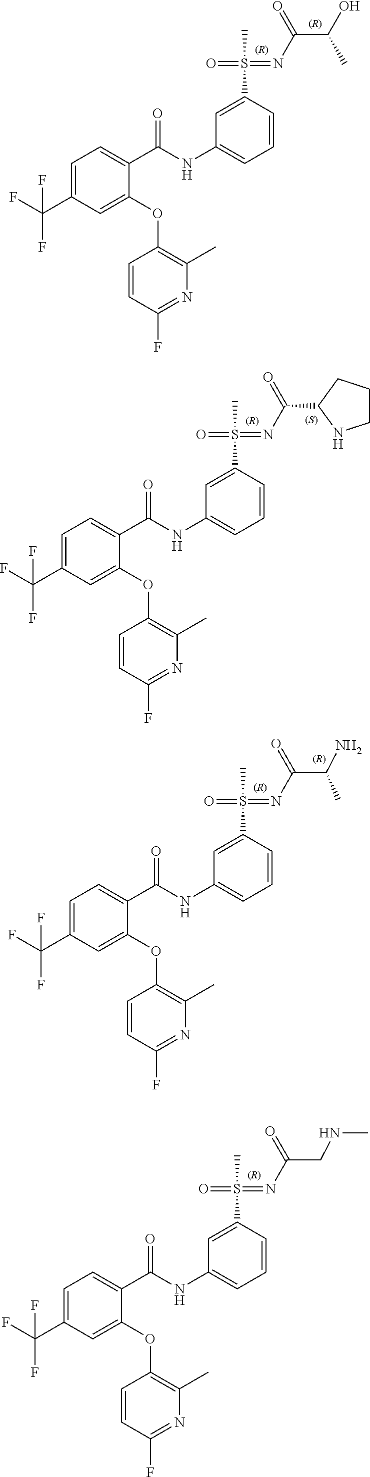

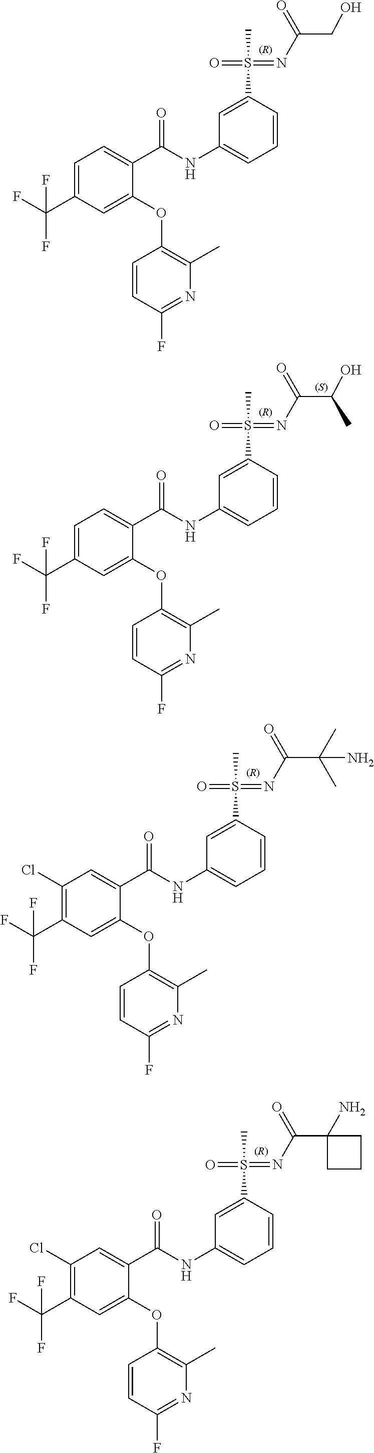

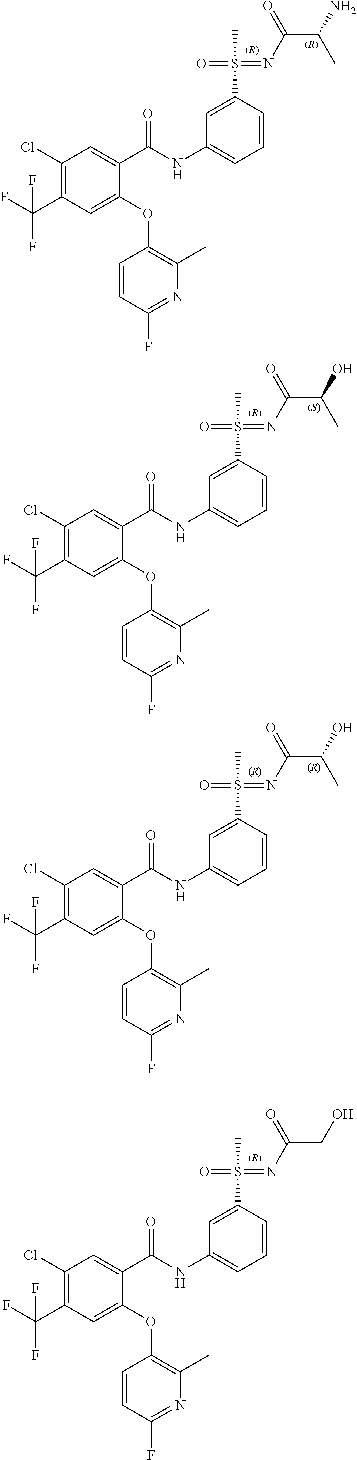

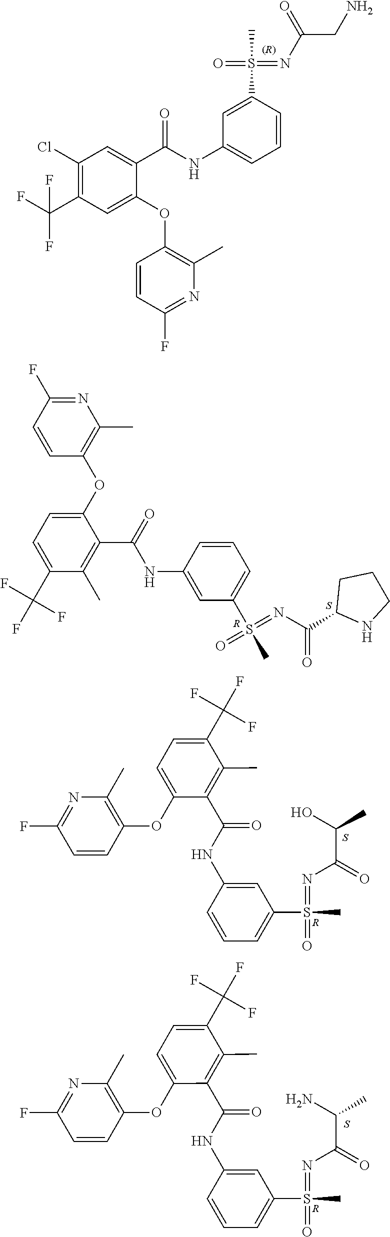

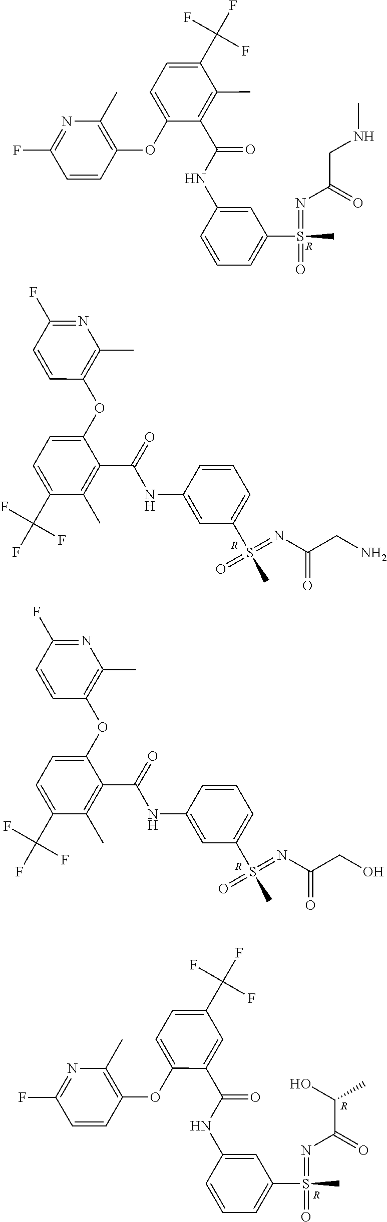

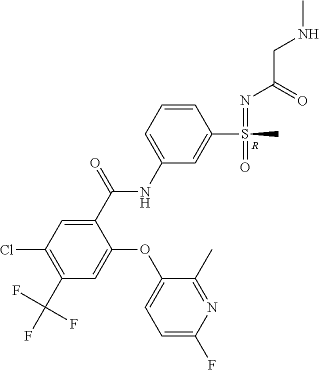

The second set of compounds, provided herein, are prepared by methods and procedures described below. The intermediates described in this section may be relied upon for preparation of second set of compounds.

Chemicals were purchased from standard commercial vendors and used as received unless otherwise noted. Otherwise, their preparation is facile and known to one of ordinary skill in the art, or it is referenced or described herein. Abbreviations are consistent with those in the ACS Style Guide. “dry” glassware means oven/desiccator dried. Solvents were ACS grade unless otherwise noted.

All reactions were performed in flame-dried or oven-dried glassware under a positive pressure of dry nitrogen or dry argon and were stirred magnetically unless otherwise indicated. Chemicals were purchased from standard commercial vendors and used as received unless otherwise noted. Yields are not optimized. The chemical names were generated using the ChemDraw Professional 19.1, available from PerkinElmer or chemAxon.

Reactions were monitored by thin layer chromatography (TLC) using 0.25 mm silica gel 60 F254 plates purchased from EMD MILLIPORE™. Purification was performed with Biotage Isolera One Flash Chromatography Instrument or purified using one of the preparative HPLC methods mentioned below.

### Prep Method 1

Equipment: Shimadzu LCMS 2020 mass-directed preparative HPLC System; column: Gemini 5 um C₁₈ column, 150*21.2 mm; General gradient: 30% to 90% MeCN/H₂O containing 0.1% HCOOH, gradient may be slight adjusted for specific compound; Flow rate: 20 mL/min; Column temperature: ambient temperature; UV Wavelength: 214 and 254 nm;

### Prep Method 2

Equipment: Shimadzu LC-20AP Preparative HPLC System; column: Gemini 5 um C₁₈ column, 150*21.2 mm; General gradient: 30% to 90% MeCN/H₂O containing 0.1% TFA, gradient may be slight adjusted for specific compound; Flow rate: 20 mL/min; Column temperature: ambient temperature; UV Wavelength: 214 and 254 nm.

### Prep Method 3

Equipment: Shimadzu LC-20AP Preparative HPLC System; column: Gemini 5 um C₁₈ column, 150×21.2 mm; General gradient: 30% to 90% MeCN/H₂O containing 0.05% ammonia, gradient may be slight adjusted for specific compound; Flow rate: 20 mL/min; Column temperature: ambient temperature; UV Wavelength: 214 and 254 nm.

Analytical LCMC were collected using one of following methods—

### Method 1

Equipment: Shimadzu LCMS 2020 Mass Spectrometer; Column: HALO C₁₈ 2.7 μm, 3.0 mm×30 mm; Mobile Phase: MeCN (0.05% HCOOH)—Water (0.05% HCOOH); Gradient: MeCN from 5% to 95% over 1.4 min, hold 0.6 min, total run time is 2.5 min; Flow rate: 1.8 mL/min; Column temperature: 50° C.; Wavelength: 214 and 254 nm PDA.

### Method 2

Equipment: Shimadzu LCMS 2020 Mass Spectrometer; XBridge BEH C₁₈ 2.5 μm, 3.0 mm×30 mm Mobile Phase: MeCN—Water (0.1% NH₄OH); Gradient: MeCN from 5% to 95% over 1.8 min, hold 0.7 min, total run time is 3.0 min; Flow rate: 1.0 mL/min; Column temperature: 50° C.; Wavelength: 214 and 254 nm PDA.

### Method 3

Equipment: Shimadzu LCMS 2020 Mass Spectrometer; Column: HALO C₁₈ 2.7 μm, 3.0 mm×30 mm Mobile Phase: MeCN (0.05% TFA)—Water (0.05% TFA); Gradient: MeCN from 5% to 95% over 1.4 min, hold 0.6 min, total run time is 2.5 min; Flow rate: 1.8 mL/min; Column temperature: 50° C.; Wavelength: 214 and 254 nm PDA.

SFC chiral resolution was performed on Shimadzu Nexera UC Preparative SFC System (SFE-30A, LC-30AD_{SF}, SFC-30A) using following methods:

### Method 1

Column: Daicel chiralpak-AS-H 5 μm 250×20 mm; Mobile Phase: CO₂/MeOH [0.1% NH₃ (7M in MeOH)], CO₂/MeOH ratio varies for different compounds; Oven temperature: 40° C.; Flow rate: 38 mL/min.

### Method 2

Column: Daicel chiralpak-OJ-H 5 μm 250*20 mm; Mobile Phase: CO₂/MeOH (0.1% HCOOH), CO₂/MeOH ratio varies for different compounds; Oven temperature: 40° C.; Flow rate: 38 mL/min.

### Method 3

Column: Daicel chiralpak-OD-H 5 μm 250×20 mm; Mobile Phase: CO₂/MeOH, ratio varies for different compounds; Oven temperature: 40° C.; Flow rate: 38 mL/min.

### Method 4

Column: Daicel chiralpak-AD-H 5 μm 250×20 mm; Mobile Phase: CO₂/i-PrOH, ratio varies for different compounds; Oven temperature: 40° C.; Flow rate: 38 mL/min.

### Method 5

Column: Daicel chiralpak-IC 5 μm 250×20 mm; Mobile Phase: CO₂/EtOH, ratio varies for different compounds; Oven temperature: 40° C.; Flow rate: 38 mL/min.

Unless otherwise stated, ¹H nuclear magnetic resonance spectroscopy (NMR) spectra were recorded on a Bruker AVANCE NEO 400 MHz Digital NMR Spectrometer. Chemical shifts, 6, are quoted in parts per million (ppm) relative to TMS and calibrated using residual un-deuterated solvent as an internal reference. The following abbreviations are used to denote the multiplicities and general assignments: s (singlet), d (doublet), t (triplet), q (quartet), dd (doublet of doublets), ddd (doublet of doublet of doublets), dt (doublet of triplets), dq (doublet of quartets), hep (heptet), m (multiplet), pent (pentet), td (triplet of doublets), qd (quartet of doublets), app. (apparent) and br. (broad). Coupling constants, J, are quoted to the nearest 0.1 Hz.

### General Synthetic Schemes

Several methods for preparing the compounds of this invention are illustrated in the following Schemes and Examples. The present invention further provides processes for the preparation of compounds of structural Formula I as defined above. In some cases, the order of carrying out the foregoing reaction schemes may be varied to facilitate the reaction or to avoid unwanted reaction products. The following examples are provided for the purpose of illustration only and are not to be construed as limitations on the disclosed invention.

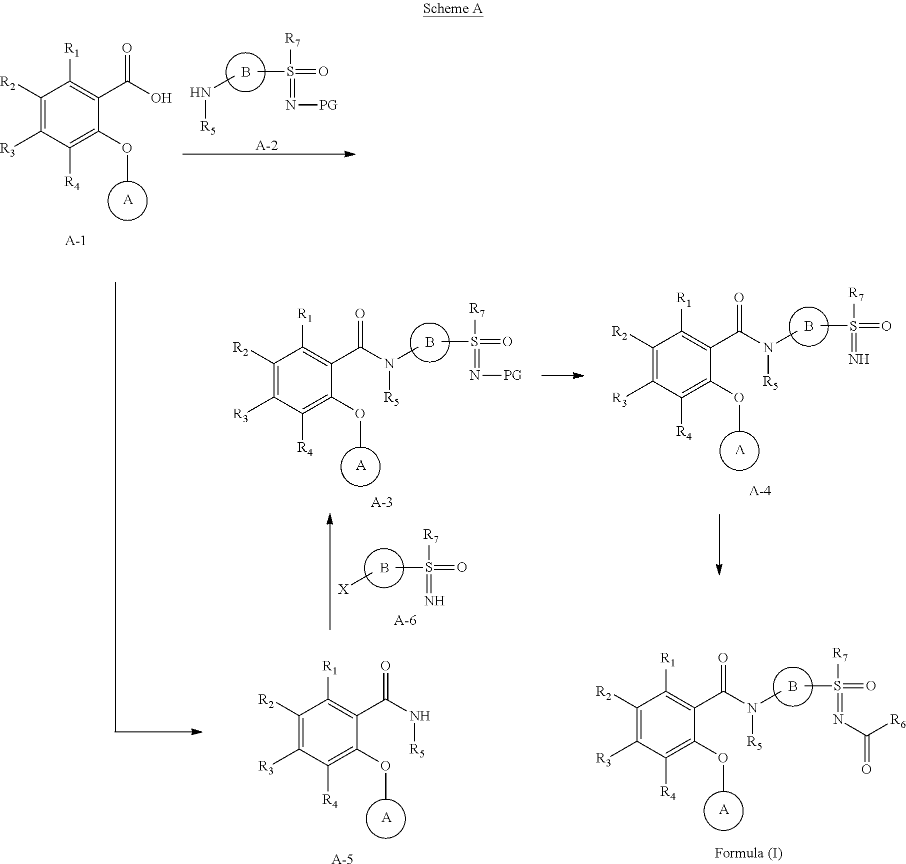

As illustrated in Scheme A, in general, compounds of Formula (I) may be synthesized starting from carboxylic acids A-1 by reacting with a substituted aniline or heteroaryl aniline A-2 using standard amide coupling reagents, not limited to HATU, TBTU, EDC or T3P in organic solvents and base, such as DIEA, to give intermediates of type A-3. Intermediates A-3 may contain a protecting group (PG) such as Boc which may be removed by treatment with an acid such as TFA to provide intermediates having structure A-4. Any suitable PG such as Cbz or Fmoc may be employed and can be removed accordingly. The NH group of A-4 may be acylated using standard amide coupling reagents such as HATU in conjunction with a carboxylic acid and a base such as DIEA or the NH group may be acylated with a reactive carboxylic acid ester such as an N-hydroxysuccinimide ester, to provide compounds of Formula (I) after removal of any protecting groups. There are numerous methods to acylate the NH group which can be affected by those skilled in the art. If desired, Compounds of Formula (I) may be further modified to provide additional Compounds of Formula (I). Alternatively, carboxylic acid A-1 may be treated with ammonia or a primary amine (R₅NH₂) in the presence of an amide coupling reagent such as HATU and a base such as DIEA to give carboxamide intermediate A-5 which can undergo metal-catalyzed coupling with a halogen-substituted aryl or heteroaryl compounds of type A-6 to give compounds of formula A-3. Compounds of Formula (I) can then be obtained from A-3 as described.

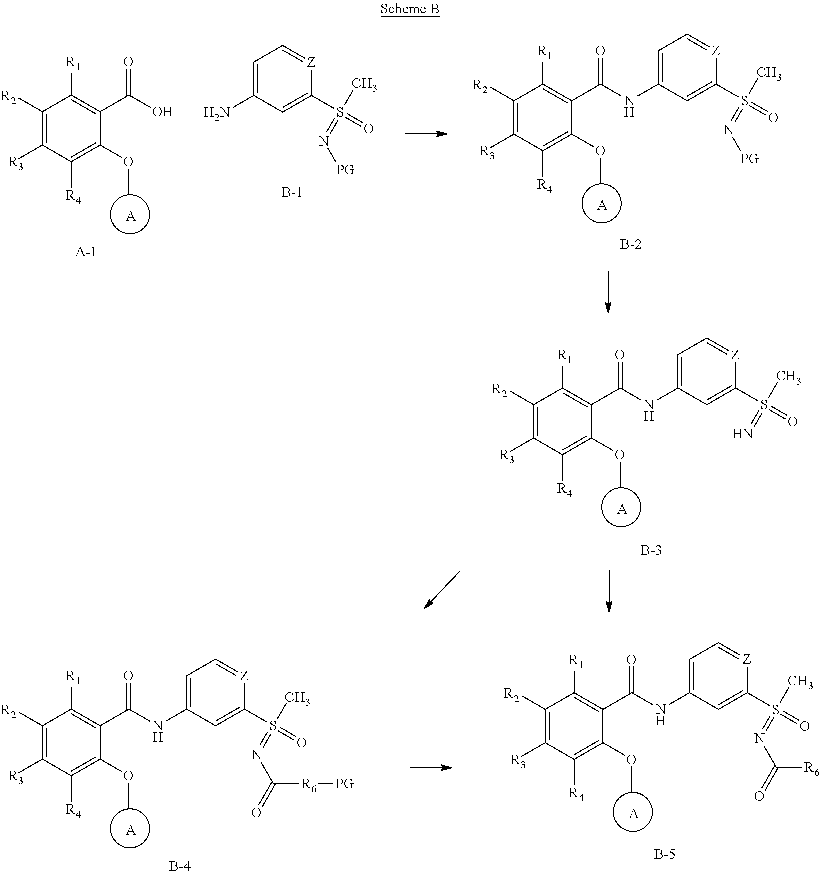

As further illustrated in Scheme B, in general, compounds of the invention can be prepared by reacting intermediates A-1 with an amine B-1, utilizing amide coupling conditions or by activation of an appropriately functionalized carboxylic acid A-1 with (COCl)₂ or POCl₃ and with amine B-1 and base such as DIEA or pyridine in DCM, DMF or THF to give compounds of type B-2. The compounds of formula B-3 can be formed by removing a protecting group, such as Boc under acidic conditions. In some instances, B-3 can be separated into the corresponding R and S isomers using chiral HPLC. R- and S-isomers also can be prepared by coupling of the acid to enantiomerically pure amine B-1 followed by deprotection. The sulfoximine nitrogen of intermediate B-3 was acylated using standard amide coupling reagents such as, but not limited to, HATU, a base such as DIEA, and HO₂C—R₆-PG to give compounds of formula B-4. When B-4 contains a PG, as shown, it was removed to give a compound of formula B-5. For example, if a Boc group as used to protect the sulfoximine, it was removed by treatment with TFA to give compounds of formula B-5. Trialkylsilyl groups may be employed to protect hydroxyl groups and can be removed by treatment with aqueous acids such as acetic acid. B-3 may yield compound B-directly if there is no need for a protecting group.

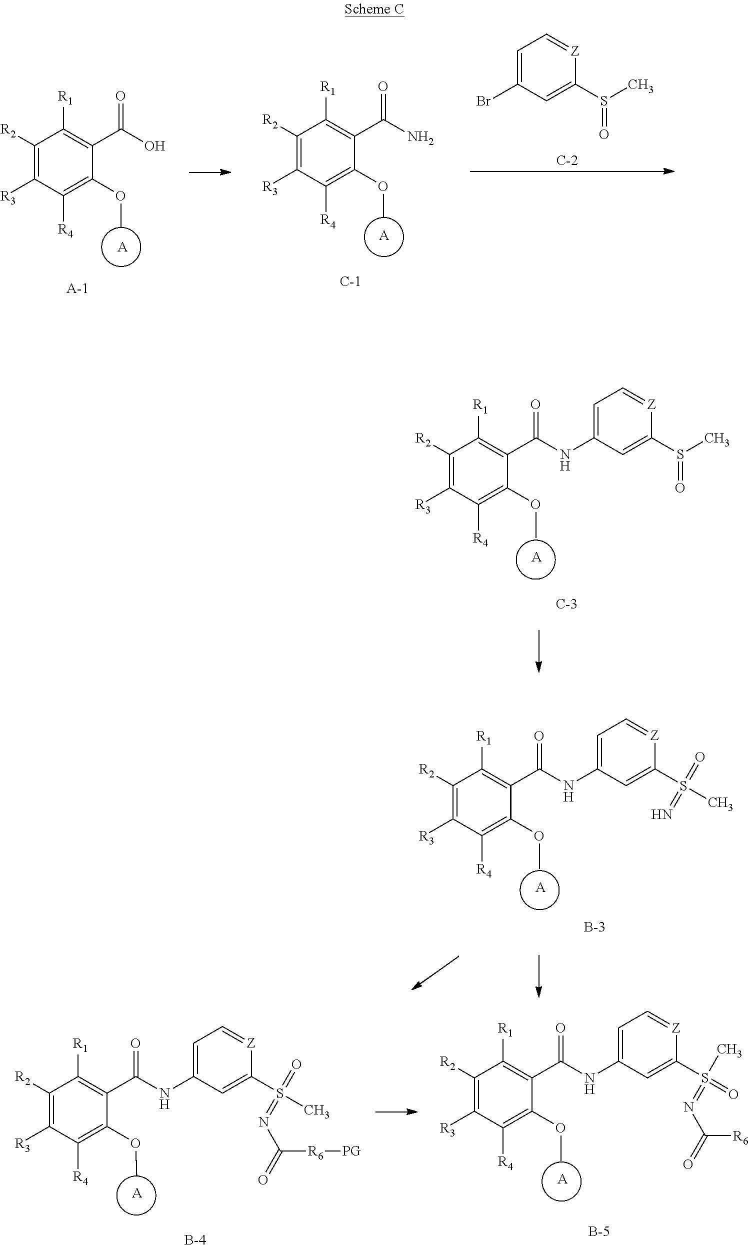

As illustrated in Scheme C, in general, compounds of the invention can be prepared by activation of appropriately functionalized carboxylic acid A-1 in organic solvent with either (COCl)₂ or SOCl₂ followed by addition of NH₄OH to afford C-1. Intermediate C-1 can then be brought together with materials of variously substituted Br compounds C-2, utilizing Xantphos-Pd-G2 mediated coupling conditions to deliver intermediate C-3. The compounds of formula C-3 may be treated with ammonium carbonate or ammonium carbamate and (diacetoxyiodo)benzene (PIDA) in methanol to provide compounds of formula B-3. In some instances, B-3 was separated to the corresponding R and S isomers using chiral HPLC conditions. Compounds of formula B-4 were obtained using standard amide coupling reagents such as, not limited to, HATU, a base such as DIEA, and HO₂C—R₆-PG. When the protecting group PG was Boc, it was removed by treatment with TFA to give compounds of formula B-5. Trialkylsilyl groups may be employed to protect hydroxyl groups and can be removed by treatment with aqueous acids such as acetic acid. B-3 may yield compound B-5 directly if there is no need for a protecting group.

The above schemes are intended to be illustrative and not limiting in any way. Those skilled in the art are able to prepare the Compounds of Formula (I) using these general schemes as a guideline but other methods are available to accomplish the synthesis of compounds of the invention. Specific methods are provided for each of the Examples to further illustrate the synthesis of Compounds of Formula (I).

### Intermediate 1

### Methyl 2-chloro-4-methyl-5-nitronicotinate

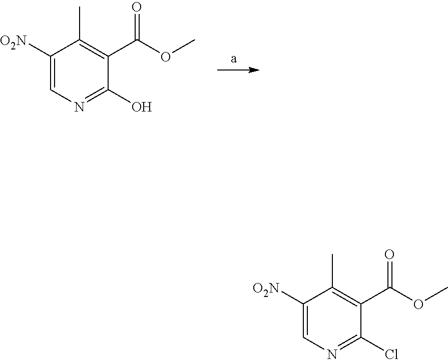

Reagents & conditions: a) phenyl dichlorophosphate, 160° C.

A solution of methyl 2-hydroxy-4-methyl-5-nitronicotinate (4.0 g, 18.9 mmol) in phenyl dichlorophosphate (20 mL) was heated to 160° C. for 3 h. The resulting solution was cooled to room temperature, quenched with water (100 mL) and extracted with EtOAc (50 mL×3). The combined organic phases were washed with brine, dried over sodium sulfate, concentrated under vacuum.

The residue was purified by flash column chromatography on silica gel (PE/EtOAc=10/1) to provide methyl 2-chloro-4-methyl-5-nitronicotinate (3 g, 69%) as a light-yellow solid. LCMS (ESI) calcd. for C₈H₈ClN₂O₄ [M+H]⁺ m/z 231.02, found 230.85.

### Intermediate 2

### (S)-2-((tert-butyldimethylsilyl)oxy)propanoic acid

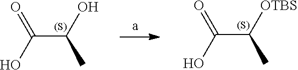

Reagents & conditions: TBSCl, imidazole, DCM

To a solution of (S)-2-hydroxypropanoic acid (1 g, 11.11 mmol) and imidazole (2.27 g, 33.32 mmol) in DCM (15 mL) was added TBSCl (2.00 g, 13.33 mmol) at 0° C. Then the mixture was heated at 25° C. for 16 hours. After the rection was completed, the mixture was diluted with water and extracted with DCM (3×30 mL). The combined organic layers were washed with brine, dried with Na₂SO₄, and concentrated under vacuum to give crude (S)-2-((tert-butyldimethylsilyl)oxy)propanoic acid (1.4 g) which was used directly in next step without further purification.

### Intermediate 3

### (R)-2-((tert-butyldimethylsilyl)oxy)propanoic acid

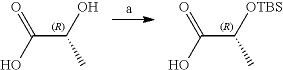

Reagents & conditions: TBSCl, imidazole, DCM

To a solution of (R)-2-hydroxypropanoic acid (1 g, 11.11 mmol) and imidazole (2.27 g, 33.32 mmol) in DCM (15 mL) was added TBSCl (2.00 g, 13.33 mmol) at 0° C. Then the mixture was warmed to RT and stirred for 16 hours. After the rection was completed, the mixture was diluted with water and extracted with DCM (3×30 mL). The combined organic layers were washed with brine, dried with Na₂SO₄, and concentrated under vacuum to give crude (R)-2-((tert-butyldimethylsilyl)oxy)propanoic acid (1.6 g) which was used directly in next step without further purification.

### Intermediate 4

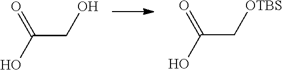 [SMILES: O=C(O)CO ; CC(C)(C)[Si](C)(C)OCC(=O)O]

2-((tert-butyldimethylsilyl)oxy)acetic acid: To a solution of 2-hydroxyacetic acid (1 g, 13.16 mmol) and imidazole (2.68 g, 39.47 mmol) in DCM (15 mL) was added TBSCl (2.38 g, 15.79 mmol) at 0° C. Then the mixture was stirred at 25° C. for 16 hours. After the rection was completed. The mixture was diluted with water and extracted with DCM (3×30 mL). The combined organic layers were washed with brine, dried with Na₂SO₄, and concentrated under vacuum to give crude 2-((tert-butyldimethylsilyl)oxy)acetic acid (1.6 g) which was used directly in next step without further purification.

### Example 1B

### 5-chloro-2-((6-fluoro-2-methylpyridin-3-yl)oxy)-N-(3-((R)—N—((R)-2-hydroxypropanoyl)-S-methylsulfonimidoyl)phenyl)-4-(trifluoromethyl)benzamide

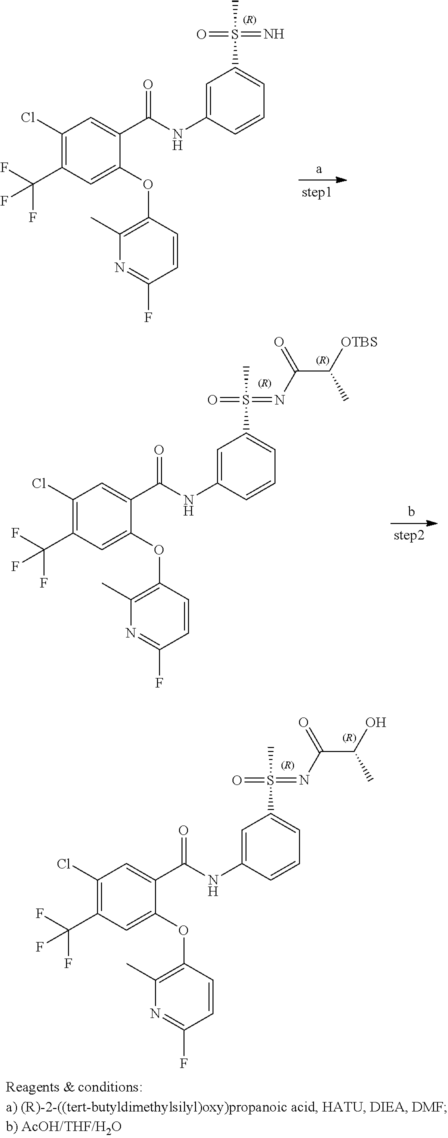

Step 1: N-(3-((R)—N—((R)-2-((tert-butyldimethylsilyl)oxy)propanoyl)-S-methylsulfonimidoyl)phenyl)-5-chloro-2-((6-fluoro-2-methylpyridin-3-yl)oxy)-4-(trifluoromethyl)benzamide: A mixture of (R)-5-chloro-2-((6-fluoro-2-methylpyridin-3-yl)oxy)-N-(3-(S-methylsulfonimidoyl)phenyl)-4-(trifluoromethyl)benzamide (160 mg, 0.32 mmol), (R)-2-((tert-butyldimethylsilyl)oxy)propanoic acid (325.7 mg, 1.6 mmol) and HATU (181.8 mg, 0.48 mmol) in DMF (5 mL) was added DIEA (123.6 mg, 0.96 mmol) at room temperature. The reaction mixture was stirred at room temperature for 16 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to provide N-(3-((R)—N—((R)-2-((tert-butyldimethylsilyl)oxy)propanoyl)-S-methylsulfonimidoyl)phenyl)-5-chloro-2-((6-fluoro-2-methylpyridin-3-yl)oxy)-4-(trifluoromethyl)benzamide (140 mg, 63.8%) as a yellow solid. LCMS (ESI) calcd. for C₃₀H₃₅ClF₄N₃O₅SSi [M+H]⁺ m/z 688.17, found 688.15.

Step 2: 5-chloro-2-((6-fluoro-2-methylpyridin-3-yl)oxy)-N-(3-((R)—N—((R)-2-hydroxypropanoyl)-S-methylsulfonimidoyl)phenyl)-4-(trifluoromethyl)benzamide: A solution of N-(3-((R)—N—((R)-2-((tert-butyldimethylsilyl)oxy)propanoyl)-S-methylsulfonimidoyl)phenyl)-5-chloro-2-((6-fluoro-2-methylpyridin-3-yl)oxy)-4-(trifluoromethyl)benzamide (140 mg, 0.2 mmol) in THF/H₂O/AcOH (1/3/3, 7 mL) was stirred at room temperature for 4 hours. After the reaction was completed, the resulting solution was diluted with water (30 mL) and extracted with EtOAc (30 mL×3). The combined organic layers were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% formic acid) to provide 5-chloro-2-((6-fluoro-2-methylpyridin-3-yl)oxy)-N-(3-((R)—N—((R)-2-hydroxypropanoyl)-S-methylsulfonimidoyl)phenyl)-4-(trifluoromethyl)benzamide (45.6 mg, 39.1%) as a white solid. ¹H NMR (400 MHz, DMSO-d₆) δ 11.07 (s, 1H), 8.34 (s, 1H), 8.12 (s, 1H), 7.89 (d, J=7.5 Hz, 1H), 7.73-7.61 (m, 3H), 7.36 (s, 1H), 7.05 (dd, J=8.7, 3.3 Hz, 1H), 4.71 (d, J=5.3 Hz, 1H), 4.02 (dd, J=6.8, 5.4 Hz, 1H), 3.46 (s, 3H), 2.33 (s, 3H), 1.26 (d, J=6.8 Hz, 3H). LCMS (ESI) calcd. for C₂₄H₂₁ClF₄N₃O₅S [M+H]⁺ m/z 574.08, found 574.00.

### Example 2B

### (R)-5-chloro-2-((6-fluoro-2-methylpyridin-3-yl)oxy)-N-(3-(N-(2-hydroxyacetyl)-S-methylsulfonimidoyl)phenyl)-4-(trifluoromethyl)benzamide

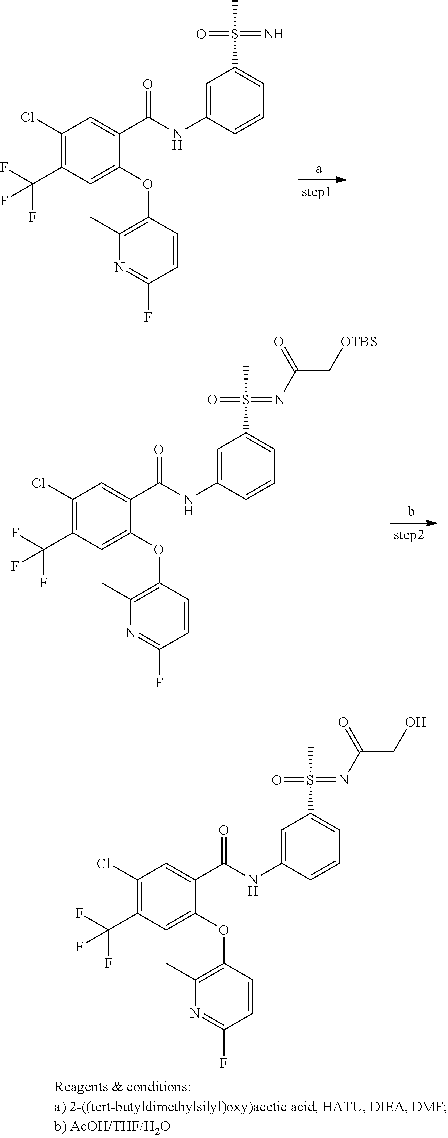 [SMILES: Cc1nc(F)ccc1Oc1cc(C(F)(F)F)c(Cl)cc1C(=O)Nc1cccc([S@](C)(=N)=O)c1 ; Cc1nc(F)ccc1Oc1cc(C(F)(F)F)c(Cl)cc1C(=O)Nc1cccc([S@@](C)(=O)=NC(=O)CO[Si](C)(C)C(C)(C)C)c1 ; Cc1nc(F)ccc1Oc1cc(C(F)(F)F)c(Cl)cc1C(=O)Nc1cccc([S@@](C)(=O)=NC(=O)CO)c1]

Step 1: (R)—N-(3-(N-(2-((tert-butyldimethylsilyl)oxy)acetyl)-S-methylsulfonimidoyl)phenyl)-5-chloro-2-((6-fluoro-2-methylpyridin-3-yl)oxy)-4-(trifluoromethyl)benzamide: A mixture of (R)-5-chloro-2-((6-fluoro-2-methylpyridin-3-yl)oxy)-N-(3-(S-methylsulfonimidoyl)phenyl)-4-(trifluoromethyl)benzamide (150 mg, 0.3 mmol), 2-((tert-butyldimethylsilyl)oxy)acetic acid (170.6 mg, 0.9 mmol) and HATU (170.5 mg, 0.45 mmol) in DMF (5 mL) was added DIEA (115.9 mg, 0.9 mmol) at room temperature. The reaction mixture was stirred at room temperature for 16 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to provide (R)—N-(3-(N-(2-((tert-butyldimethylsilyl)oxy)acetyl)-S-methylsulfonimidoyl)phenyl)-5-chloro-2-((6-fluoro-2-methylpyridin-3-yl)oxy)-4-(trifluoromethyl)benzamide (120 mg, 53.6%) as a yellow solid. LCMS (ESI) calcd. for C₂₉H₃₃ClF₄N₃O₅SSi [M+H]⁺ m/z 674.15, found 674.05.

Step 2: (R)-5-chloro-2-((6-fluoro-2-methylpyridin-3-yl)oxy)-N-(3-(N-(2-hydroxyacetyl)-S-methylsulfonimidoyl)phenyl)-4-(trifluoromethyl)benzamide: A solution of (R)—N-(3-(N-(2-((tert-butyldimethylsilyl)oxy)acetyl)-S-methylsulfonimidoyl)phenyl)-5-chloro-2-((6-fluoro-2-methylpyridin-3-yl)oxy)-4-(trifluoromethyl)benzamide (120 mg, 0.18 mmol) in THF/H₂O/AcOH (1/3/3, 7 mL) was stirred at room temperature for 4 hours. After the reaction was completed, the resulting solution was diluted with water (30 mL) and extracted with EtOAc (30 mL×3). Then the organic solution was washed with water (20 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 90% MeCN/H₂O containing 0.1% FA) to provide (R)-5-chloro-2-((6-fluoro-2-methylpyridin-3-yl)oxy)-N-(3-(N-(2-hydroxyacetyl)-S-methylsulfonimidoyl)phenyl)-4-(trifluoromethyl)benzamide (45.4 mg, 45.6%) as a white solid. ¹H NMR (400 MHz, DMSO-d₆) δ 11.08 (s, 1H), 8.32 (s, 1H), 8.13 (s, 1H), 7.91 (d, J=6.8 Hz, 1H), 7.75-7.60 (m, 3H), 7.36 (s, 1H), 7.10-7.01 (m, 1H), 4.79 (d, J=5.5 Hz, 1H), 3.95 (d, J=4.9 Hz, 2H), 3.45 (s, 3H), 2.34 (s, 3H). LCMS (ESI) calcd. for C₂₃H₁₉ClF₄N₃O₅S [M+H]⁺ m/z 560.06, found 560.10.

### Example 3B

### 5-chloro-2-((6-fluoro-2-methylpyridin-3-yl)oxy)-N-(3-((R)—N—((S)-2-hydroxypropanoyl)-S-methylsulfonimidoyl)phenyl)-4-(trifluoromethyl)benzamide

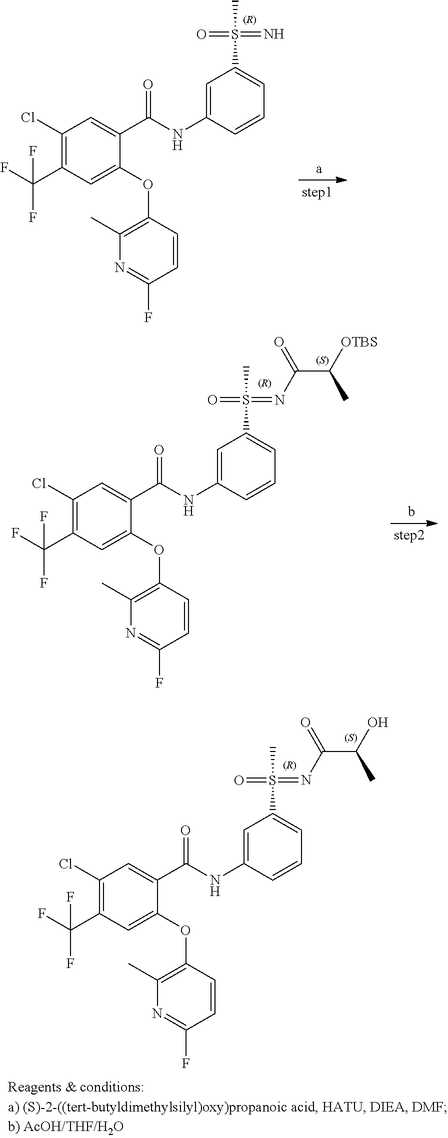

Step 1: N-(3-((R)—N—((S)-2-((tert-butyldimethylsilyl)oxy)propanoyl)-S-methylsulfonimidoyl)phenyl)-5-chloro-2-((6-fluoro-2-methylpyridin-3-yl)oxy)-4-(trifluoromethyl)benzamide. A solution of (R)-5-chloro-2-((6-fluoro-2-methylpyridin-3-yl)oxy)-N-(3-(S-methylsulfonimidoyl)phenyl)-4-(trifluoromethyl)benzamide (150 mg, 0.30 mmol), (S)-2-((tert-butyldimethylsilyl)oxy)propanoic acid (183 mg, 0.90 mmol), HATU (227 mg, 0.60 mmol) and DIEA (116 mg, 0.90 mmol) in DMF (10 mL) was stirred at room temperature for 16 hours. LCMS showed the reaction was completed. The mixture was diluted with water (30 mL) and extracted with EtOAc (30 mL×3). The combined organic layers were washed with brine, dried over sodium sulfate, concentrated under vacuum. The residue was purified by flash column chromatography on silica gel (PE/EtOAc=3/1) to provide N-(3-((R)—N—((S)-2-((tert-butyldimethylsilyl)oxy)propanoyl)-S-methylsulfonimidoyl)phenyl)-5-chloro-2-((6-fluoro-2-methylpyridin-3-yl)oxy)-4-(trifluoromethyl)benzamide (180 mg, 87.550) as colorless oil, LCMS (ESI) calcd. for C₃₀H₃₅ClF₄N₃O₅Si [M+H]⁺ m/z 688.17, found 688.15.

Step 2: 5-chloro-2-((6-fluoro-2-methylpyridin-3-yl)oxy)-N-(3-((R)—N—((S)-2-hydroxypropanoyl)-S-methylsulfonimidoyl)phenyl)-4-(trifluoromethyl)benzamide: A solution of N-(3-((R)—N—((S)-2-((tert-butyldimethylsilyl)oxy)propanoyl)-S-methylsulfonimidoyl)phenyl)-5-chloro-2-((6-fluoro-2-methylpyridin-3-yl)oxy)-4-(trifluoromethyl)benzamide (180 mg, 0.26 mmol) in THE (2.2 mL) and H₂O (6.5 mL) was added AcOH (8.5 mL) at room temperature. The reaction mixture was stirred at room temperature for 3 hours. After the reaction was completed, the mixture was diluted with water (10 mL) and extracted with EtOAc (20 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% NH₃·H₂O) to provide 5-chloro-2-((6-fluoro-2-methylpyridin-3-yl)oxy)-N-(3-((R)—N—((S)-2-hydroxypropanoyl)-S-methylsulfonimidoyl)phenyl)-4-(trifluoromethyl)benzamide (71.97 mg, 45.9%) as a white solid. ¹H NMR (400 MHz, DMSO-d₆) δ 11.07 (s, 1H), 8.35 (s, 1H), 8.12 (s, 1H), 7.87 (d, J=6.7 Hz, 1H), 7.72-7.62 (m, 3H), 7.36 (s, 1H), 7.05 (dd, J=8.7, 3.4 Hz, 1H), 4.69 (d, J=5.5 Hz, 1H), 4.08-3.96 (m, 1H), 3.44 (s, 3H), 2.34 (s, 3H), 1.24 (d, J=6.8 Hz, 3H). LCMS (ESI) calcd. for C₂₄H₂₁ClF₄N₃O₅S [M+H]⁺ m/z 574.08, found 573.95.

### Example 4B

### (R)-5-chloro-2-((6-fluoro-2-methylpyridin-3-yl)oxy)-N-(3-(N-glycyl-S-methylsulfonimidoyl)phenyl)-4-(trifluoromethyl)benzamide

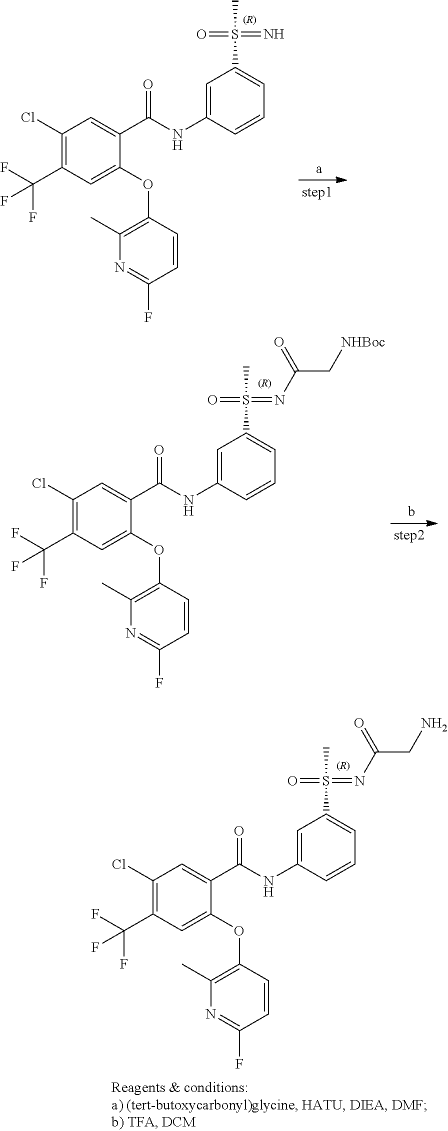

Step 1: tert-butyl (R)-(2-(((3-(5-chloro-2-((6-fluoro-2-methylpyridin-3-yl)oxy)-4-(trifluoromethyl)benzamido)phenyl)(methyl)(oxo)-λ⁶-sulfaneylidene)amino)-2-oxoethyl)carbamate: A mixture of (R)-5-chloro-2-((6-fluoro-2-methylpyridin-3-yl)oxy)-N-(3-(S-methylsulfonimidoyl)phenyl)-4-(trifluoromethyl)benzamide (120 mg, 0.24 mmol), (tert-butoxycarbonyl)glycine (83.8 mg, 0.48 mmol) and HATU (136.4 mg, 0.36 mmol) in DMF (5 mL) was added DIEA (92.7 mg, 0.72 mmol) at room temperature. The reaction mixture was stirred at room temperature for 16 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to provide tert-butyl (R)-(2-(((3-(5-chloro-2-((6-fluoro-2-methylpyridin-3-yl)oxy)-4-(trifluoromethyl)benzamido)phenyl)(methyl)(oxo)-λ⁶-sulfaneylidene)amino)-2-oxoethyl)carbamate (100 mg, 63.5%) as a yellow solid. LCMS (ESI) calcd. for C₂₈H₂₈ClF₄N₄O₆S [M+H]⁺ m/z 659.14, found 659.10.

Step 2 (R)-5-chloro-2-((6-fluoro-2-methylpyridin-3-yl)oxy)-N-(3-(N-glycyl-S-methylsulfonimidoyl)phenyl)-4-(trifluoromethyl)benzamide: A solution of tert-butyl (R)-(2-(((3-(5-chloro-2-((6-fluoro-2-methylpyridin-3-yl)oxy)-4-(trifluoromethyl)benzamido)phenyl)(methyl) (oxo)-λ⁶-6-sulfaneylidene)amino)-2-oxoethyl)carbamate (100 mg, 0.15 mmol) in DCM (3 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini um C₁₈ column, 150*21.2 mm, eluting with 50% to 95% MeCN/H₂O containing 0.05% NH₃) to provide (R)-5-chloro-2-((6-fluoro-2-methylpyridin-3-yl)oxy)-N-(3-(N-glycyl-S-methylsulfonimidoyl)phenyl)-4-(trifluoromethyl)benzamide (53.8 mg, 62.5%) as a white solid. ¹H NMR (400 MHz, DMSO-d₆) δ 11.06 (s, 1H), 8.31 (s, 1H), 8.13 (s, 1H), 7.89 (d, J=7.6 Hz, 1H), 7.72-7.61 (m, 3H), 7.36 (s, 1H), 7.05 (dd, J=8.7, 3.2 Hz, 1H), 3.43 (s, 3H), 3.23 (s, 2H), 2.34 (s, 3H), 1.69 (bs, 2H). LCMS (ESI) calcd. for C₂₃H₂₀ClF₄N₄O₄S [M+H]⁺ m/z 559.09, found 559.05.

### Example 5B

### N-(3-((R)—N-(D-alanyl)-S-methylsulfonimidoyl)phenyl)-5-chloro-2-((6-fluoro-2-methylpyridin-3-yl)oxy)-4-(trifluoromethyl)benzamide

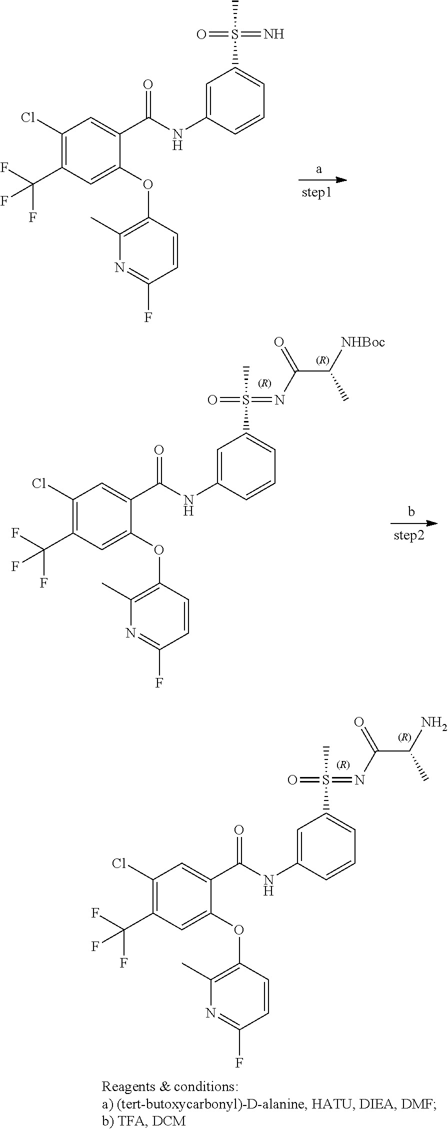

Step 1: tert-butyl ((R)-1-(((R)-(3-(5-chloro-2-((6-fluoro-2-methylpyridin-3-yl)oxy)-4-(trifluoromethyl)benzamido)phenyl)(methyl)(oxo)-λ⁶-sulfaneylidene)amino)-1-oxopropan-2-yl)carbamate: A mixture of (R)-5-chloro-2-((6-fluoro-2-methylpyridin-3-yl)oxy)-N-(3-(S-methylsulfonimidoyl)phenyl)-4-(trifluoromethyl)benzamide (120 mg, 0.24 mmol), (tert-butoxycarbonyl)-D-alanine (90.5 mg, 0.48 mmol) and HATU (136.4 mg, 0.36 mmol) in DMF (5 mL) was added DIEA (92.7 mg, 0.72 mmol) at room temperature. The reaction mixture was stirred at room temperature for 4 hours. After the reaction was completed, the resulting solution was diluted with water (60 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to provide tert-butyl ((R)-1-(((R)-(3-(5-chloro-2-((6-fluoro-2-methylpyridin-3-yl)oxy)-4-(trifluoromethyl)benzamido)phenyl)(methyl)(oxo)-λ⁶-sulfaneylidene)amino)-1-oxopropan-2-yl)carbamate (120 mg, 74.6%) as a yellow solid. LCMS (ESI) calcd. for C₂₉H₃₀ClF₄N₄O₆S [M+H]⁺ m/z 673.15, found 673.10.

Step 2: N-(3-((R)—N-(D-alanyl)-S-methylsulfonimidoyl)phenyl)-5-chloro-2-((6-fluoro-2-methylpyridin-3-yl)oxy)-4-(trifluoromethyl)benzamide: A solution of tert-butyl ((R)-1-(((R)-(3-(5-chloro-2-((6-fluoro-2-methylpyridin-3-yl)oxy)-4-(trifluoromethyl)benzamido)phenyl)(methyl)(oxo)-λ⁶-sulfaneylidene)amino)-1-oxopropan-2-yl)carbamate (120 mg, 0.18 mmol) in DCM (3 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 45% to 95% MeCN/H₂O containing 0.05% NH₃) to provide N-(3-((R)—N-(D-alanyl)-S-methylsulfonimidoyl)phenyl)-5-chloro-2-((6-fluoro-2-methylpyridin-3-yl)oxy)-4-(trifluoromethyl)benzamide (73.9 mg, 72.4%) as a white solid. ¹H NMR (400 MHz, DMSO-d₆) δ 11.07 (s, 1H), 8.33 (s, 1H), 8.12 (s, 1H), 7.88 (d, J=7.7 Hz, 1H), 7.73-7.61 (m, 3H), 7.35 (s, 1H), 7.05 (dd, J=8.7, 3.4 Hz, 1H), 3.43 (s, 3H), 3.28 (d, J=6.9 Hz, 1H), 2.34 (s, 3H), 2.04-1.31 (m, 2H), 1.16 (d, J=7.5 Hz, 3H). LCMS (ESI) calcd. for C₂₄H₂₂ClF₄N₄O₄S [M+H]⁺ m/z 573.10, found 573.10.

### Example 6B

### (R)—N-(3-(N-(1-aminocyclobutane-1-carbonyl)-S-methylsulfonimidoyl)phenyl)-5-chloro-2-((6-fluoro-2-methylpyridin-3-yl)oxy)-4-(trifluoromethyl)benzamide

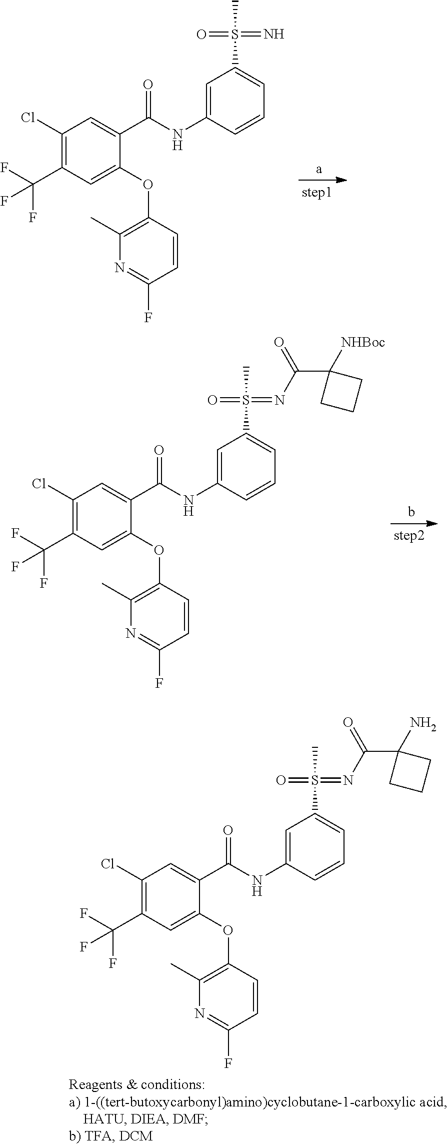 [SMILES: Cc1nc(F)ccc1Oc1cc(C(F)(F)F)c(Cl)cc1C(=O)Nc1cccc([S@](C)(=N)=O)c1 ; Cc1nc(F)ccc1Oc1cc(C(F)(F)F)c(Cl)cc1C(=O)Nc1cccc([S@@](C)(=O)=NC(=O)C2(NC(=O)OC(C)(C)C)CCC2)c1 ; Cc1nc(F)ccc1Oc1cc(C(F)(F)F)c(Cl)cc1C(=O)Nc1cccc([S@@](C)(=O)=NC(=O)C2(N)CCC2)c1]

Step 1: tert-butyl (R)-(1-(((3-(5-chloro-2-((6-fluoro-2-methylpyridin-3-yl)oxy)-4-(trifluoromethyl)benzamido)phenyl)(methyl)(oxo)-λ⁶-sulfaneylidene)carbamoyl)cyclobutyl) carbamate: A mixture of (R)-5-chloro-2-((6-fluoro-2-methylpyridin-3-yl)oxy)-N-(3-(S-methylsulfonimidoyl)phenyl)-4-(trifluoromethyl)benzamide (120 mg, 0.24 mmol), 1-((tert-butoxycarbonyl)amino)cyclobutane-1-carboxylic acid (102.9 mg, 0.48 mmol) and HATU (136.4 mg, 0.36 mmol) in DMF (5 mL) was added DIEA (92.7 mg, 0.72 mmol) at room temperature. The reaction mixture was stirred at 50° C. for 16 hours. After the reaction was completed, the resulting solution was diluted with water (50 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to provide tert-butyl(R)-(1-(((3-(5-chloro-2-((6-fluoro-2-methylpyridin-3-yl)oxy)-4-(trifluoromethyl)benzamido)phenyl)(methyl)(oxo)-λ⁶-sulfaneylidene)carbamoyl)cyclobutyl)carbamate (120 mg, 71.8%) as a yellow solid. LCMS (ESI) calcd. for C₃₁H₃₂ClF₄N₄O₆S [M+H]⁺ m/z 699.17, found 699.15.

Step 2: (R)—N-(3-(N-(1-aminocyclobutane-1-carbonyl)-S-methylsulfonimidoyl)phenyl)-5-chloro-2-((6-fluoro-2-methylpyridin-3-yl)oxy)-4-(trifluoromethyl)benzamide. A solution of tert-butyl (R)-(1-(((3-(5-chloro-2-((6-fluoro-2-methylpyridin-3-yl)oxy)-4-(trifluoromethyl)benzamido)phenyl)(methyl)(oxo)-λ⁶-sulfaneylidene)carbamoyl)cyclobutyl)carbamate (120 mg, 0.17 mmol) in DCM (3 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% NH₃) to provide (R)—N-(3-(N-(1-aminocyclobutane-1-carbonyl)-S-methylsulfonimidoyl)phenyl)-5-chloro-2-((6-fluoro-2-methylpyridin-3-yl)oxy)-4-(trifluoromethyl)benzamide (15.70 mg, 15.4%) as a white solid. ¹H NMR (400 MHz, DMSO-d₆) δ 11.07 (s, 1H), 8.38 (s, 1H), 8.12 (s, 1H), 7.88 (d, J=7.7 Hz, 1H), 7.75-7.63 (m, 3H), 7.37 (s, 1H), 7.05 (dd, J=8.7, 3.3 Hz, 1H), 3.46 (s, 3H), 2.46-2.37 (m, 2H), 2.34 (s, 3H), 2.14-1.91 (m, 2H), 1.88-1.65 (m, 4H). LCMS (ESI) calcd. for C₂₆H₂₄ClF₄N₄O₄S [M+H]⁺ m/z 599.12, found 599.05.

### Example 7B

### (R)—N-(3-(N-(2-amino-2-methylpropanoyl)-S-methylsulfonimidoyl)phenyl)-5-chloro-2-((6-fluoro-2-methylpyridin-3-yl)oxy)-4-(trifluoromethyl)benzamide

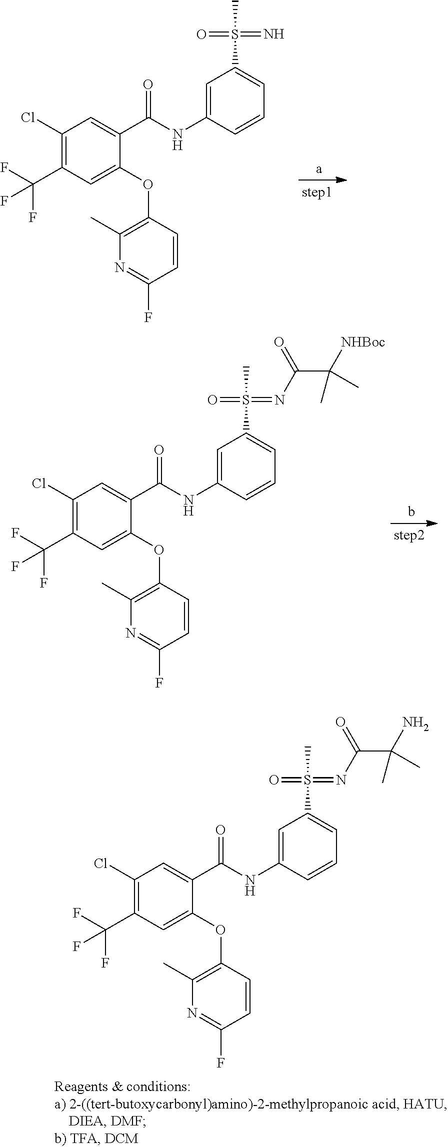 [SMILES: Cc1nc(F)ccc1Oc1cc(C(F)(F)F)c(Cl)cc1C(=O)Nc1cccc([S@](C)(=N)=O)c1 ; Cc1nc(F)ccc1Oc1cc(C(F)(F)F)c(Cl)cc1C(=O)Nc1cccc([S@@](C)(=O)=NC(=O)C(C)(C)NC(=O)OC(C)(C)C)c1 ; Cc1nc(F)ccc1Oc1cc(C(F)(F)F)c(Cl)cc1C(=O)Nc1cccc([S@@](C)(=O)=NC(=O)C(C)(C)N)c1]

Step 1: tert-butyl (R)-(1-(((3-(5-chloro-2-((6-fluoro-2-methylpyridin-3-yl)oxy)-4-(trifluoromethyl)benzamido)phenyl)(methyl)(oxo)-λ⁶-sulfaneylidene)amino)-2-methyl-1-oxopropan-2-yl)carbamate: A mixture of (R)-5-chloro-2-((6-fluoro-2-methylpyridin-3-yl)oxy)-N-(3-(S-methylsulfonimidoyl)phenyl)-4-(trifluoromethyl)benzamide (120 mg, 0.24 mmol), 2-((tert-butoxycarbonyl)amino)-2-methylpropanoic acid (243 mg, 1.2 mmol) and HATU (181.8 mg, 0.48 mmol) in DMF (5 mL) was added DIEA (154.5 mg, 1.2 mmol) at room temperature. The reaction mixture was heated at 50° C. for 16 hours. After the reaction was completed, the resulting solution was diluted with water (50 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to provide tert-butyl (R)-(1-(((3-(5-chloro-2-((6-fluoro-2-methylpyridin-3-yl)oxy)-4-(trifluoromethyl)benzamido)phenyl)(methyl)(oxo)-λ⁶-sulfaneylidene)amino)-2-methyl-1-oxopropan-2-yl)carbamate (100 mg, 60.9%) as a yellow solid. LCMS (ESI) calcd. for C₃₀H₃₂ClF₄N₄O₆S [M+H]⁺ m/z 687.17, found 687.10.

Step 2: (R)—N-(3-(N-(2-amino-2-methylpropanoyl)-S-methylsulfonimidoyl)phenyl)-5-chloro-2-((6-fluoro-2-methylpyridin-3-yl)oxy)-4-(trifluoromethyl)benzamide: A solution of tert-butyl (R)-(1-(((3-(5-chloro-2-((6-fluoro-2-methylpyridin-3-yl)oxy)-4-(trifluoromethyl)benzamido)phenyl)(methyl)(oxo)-λ⁶-sulfaneylidene)amino)-2-methyl-1-oxopropan-2-yl)carbamate (100 mg, 0.15 mmol) in DCM (3 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% NH₃) to provide (R)—N-(3-(N-(2-amino-2-methylpropanoyl)-S-methylsulfonimidoyl)phenyl)-5-chloro-2-((6-fluoro-2-methylpyridin-3-yl)oxy)-4-(trifluoromethyl)benzamide (47.2 mg, 55.3%) as a white solid. ¹H NMR (400 MHz, DMSO-d₆) δ 11.05 (s, 1H), 8.34 (s, 1H), 8.11 (s, 1H), 7.86 (d, J=7.4 Hz, 1H), 7.71-7.62 (m, 3H), 7.37 (s, 1H), 7.04 (dd, J=8.7, 3.4 Hz, 1H), 3.42 (s, 3H), 2.34 (s, 3H), 1.96-1.50 (m, 2H), 1.19 (d, J=3.3 Hz, 6H). LCMS (ESI) calcd. for C₂₅H₂₄ClF₄N₄O₄S [M+H]⁺ m/z 587.12, found 587.10.

### Example 8B

### (S)—N—((R)-(3-(2-((6-fluoro-2-methylpyridin-3-yl)oxy)-4-(trifluoromethyl)benzamido)phenyl)(methyl)(oxo)-λ⁶-sulfaneylidene)pyrrolidine-2-carboxamide formate

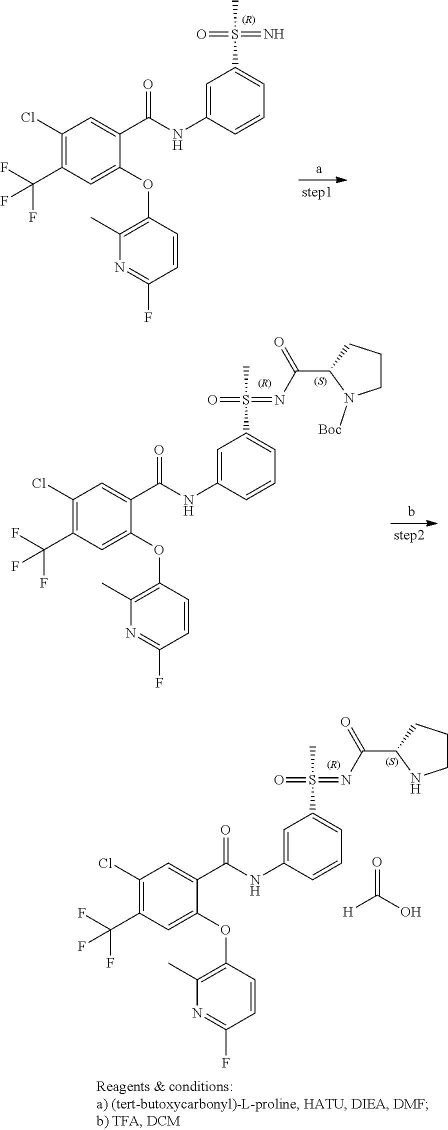

Step 1: A mixture of (R)-2-((6-fluoro-2-methylpyridin-3-yl)oxy)-N-(3-(S-methylsulfonimidoyl)phenyl)-4-(trifluoromethyl)benzamide (150 mg, 0.32 mmol) and (tert-butoxycarbonyl)-L-proline (139 mg, 0.64 mmol) in DMF (5 mL) was added HATU (183 mg, 0.48 mmol) and DIEA (124 mg, 0.96 mmol). The mixture was stirred at room temperature for 16 hours. The reaction was monitored by LCMS. After the reaction was completed, the resulting solution was diluted with water (40 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give tert-butyl (S)-2-(((R)-(3-(2-((6-fluoro-2-methylpyridin-3-yl)oxy)-4-(trifluoromethyl)benzamido)phenyl)(methyl)(oxo)-λ⁶-sulfaneylidene)carbamoyl)pyrrolidine-1-carboxylate (120 mg, 50.6%) as a yellow solid. LCMS (ESI) calcd. for C₃₁H₃₃F₄N₄O₆S [M+H]⁺ m/z 665.21, found 665.10.

Step 2: A solution of tert-butyl (S)-2-(((R)-(3-(2-((6-fluoro-2-methylpyridin-3-yl)oxy)-4-(trifluoromethyl)benzamido)phenyl)(methyl)(oxo)-λ⁶-sulfaneylidene)carbamoyl)pyrrolidine-1-carboxylate (120 mg, 0.18 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 95% MeCN/H₂O containing 0.05% NH₃) to obtain (S)—N—((R)-(3-(2-((6-fluoro-2-methylpyridin-3-yl)oxy)-4-(trifluoromethyl)benzamido)phenyl) (methyl)(oxo)-λ⁶-sulfaneylidene)pyrrolidine-2-carboxamide formate (75.6 mg, 72.8%) as a white solid. ¹H NMR (400 MHz, DMSO-d₆) δ11.07 (s, 1H), 8.43 (d, J=1.8 Hz, 1H), 8.23 (s, 1H), 7.93-7.82 (m, 2H), 7.72-7.64 (m, 4H), 7.24 (s, 1H), 7.07 (dd, J=8.7, 3.3 Hz, 1H), 3.86 (dd, J=8.5, 6.5 Hz, 1H), 3.49 (s, 3H), 3.03-2.79 (m, 2H), 2.33 (s, 3H), 2.18-2.05 (m, 1H), 1.95-1.82 (m, 1H), 1.77-1.56 (m, 2H). LCMS (ESI) calcd. for C₂₆H₂₅F₄N₄O₄S [M+H]⁺ m/z 565.16, found 565.05.

### Example 9B

### N-(3-((R)—N-(D-alanyl)-S-methylsulfonimidoyl)phenyl)-2-((6-fluoro-2-methylpyridin-3-yl)oxy)-4-(trifluoromethyl)benzamide

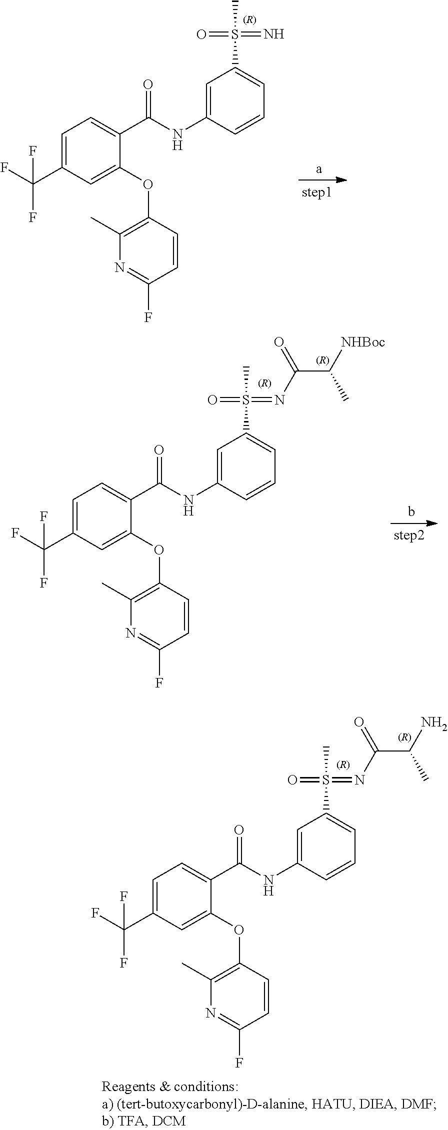

Step 1: A mixture of (R)-2-((6-fluoro-2-methylpyridin-3-yl)oxy)-N-(3-(S-methylsulfonimidoyl)phenyl)-4-(trifluoromethyl)benzamide (150 mg, 0.32 mmol) and (tert-butoxycarbonyl)-D-alanine (61 mg, 0.55 mmol) in DMF (5 mL) was added HATU (158 mg, 0.42 mmol) and DIEA (133 mg, 1.02 mmol). The mixture was stirred at room temperature for 4 hours. After the reaction was completed, the resulting solution was diluted with water (40 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give tert-butyl ((R)-1-(((R)-(3-(2-((6-fluoro-2-methylpyridin-3-yl)oxy)-4-(trifluoromethyl)benzamido)phenyl)(methyl)(oxo)-16-sulfaneylidene)amino)-1-oxopropan-2-yl)carbamate (90 mg, 43.90% yield) as a white oil. LCMS (ESI) calcd. for C₂₉H₃₁F₄N₄O₆S [M+H]⁺ m/z 639.19, found 639.10.

Step 2: A solution of tert-butyl ((R)-1-(((R)-(3-(2-((6-fluoro-2-methylpyridin-3-yl)oxy)-4-(trifluoromethyl)benzamido)phenyl)(methyl)(oxo)-λ⁶-sulfaneylidene)amino)-1-oxopropan-2-yl)carbamate (90 mg, 0.15 mmol) in DCM (10 mL) was added TFA (1 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% formic acid) to obtain N-(3-((R)—N-(D-alanyl)-S-methylsulfonimidoyl)phenyl)-2-((6-fluoro-2-methylpyridin-3-yl)oxy)-4-(trifluoromethyl)benzamide (40 mg, 53.3%) as a white solid. ¹H NMR (400 MHz, DMSO-d₆) δ 11.00 (s, 1H), 8.37 (s, 1H), 7.91-7.89 (d, J=7.6 Hz, 2H), 7.69-7.62 (m, 4H), 7.24 (s, 1H), 7.08-7.05 (m, 1H), 3.43 (s, 3H), 2.33 (s, 3H), 1.17 (d, J=7.2 Hz, 3H). LCMS (ESI) calcd. for C₂₄H₂₃F₄N₄O₄S [M+H]⁺ m/z 539.14, found 539.05.

### Example 10B

### (R)-2-((6-fluoro-2-methylpyridin-3-yl)oxy)-N-(3-(S-methyl-N-(methylglycyl)sulfonimidoyl)phenyl)-4-(trifluoromethyl)benzamide

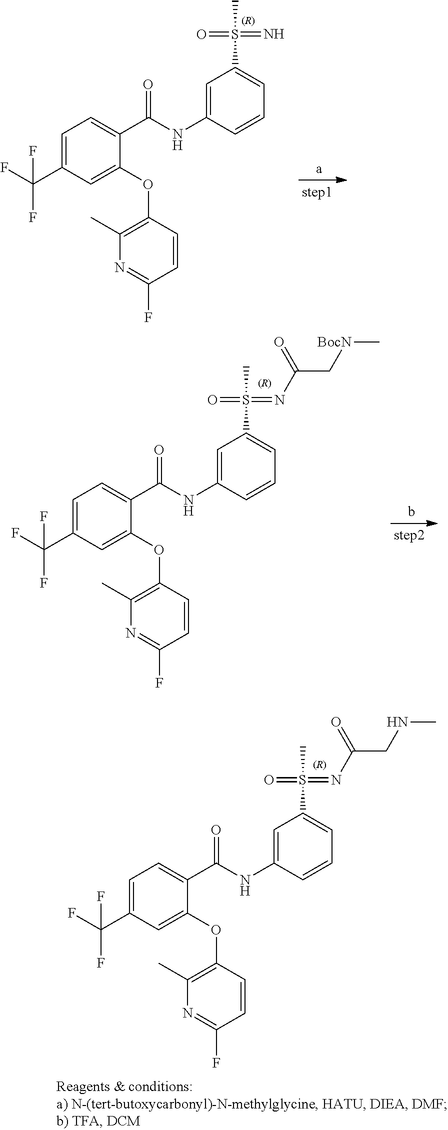

Step 1: A mixture of (R)-2-((6-fluoro-2-methylpyridin-3-yl)oxy)-N-(3-(S-methylsulfonimidoyl)phenyl)-4-(trifluoromethyl)benzamide (150 mg, 0.32 mmol) and N-(tert-butoxycarbonyl)-N-methylglycine (104 mg, 0.55 mmol) in DMF (5 mL) was added HATU (158 mg, 0.42 mmol) and DIEA (133 mg, 1.0 mmol). The mixture was stirred at room temperature for 4 hours. The reaction was monitored by LCMS. After the reaction was completed, the resulting solution was diluted with water (40 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give tert-butyl (R)-(2-(((3-(2-((6-fluoro-2-methylpyridin-3-yl)oxy)-4-(trifluoromethyl)benzamido)phenyl)(methyl)(oxo)-λ⁶-sulfaneylidene)amino)-2-oxoethyl)(methyl)carbamate (110 mg, 53.65%) as a white oil. LCMS (ESI) calcd. for C₂₉H₃₁F₄N₄O₆S [M+H]⁺ m/z 639.19, found 639.05.

Step 2: A solution of tert-butyl (R)-(2-(((3-(2-((6-fluoro-2-methylpyridin-3-yl)oxy)-4-(trifluoromethyl)benzamido)phenyl)(methyl)(oxo)-λ⁶-sulfaneylidene)amino)-2-oxoethyl)(methyl)carbamate (110 mg, 0.17 mmol) in DCM (10 mL) was added TFA (1 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% formic acid) to obtain (R)-2-((6-fluoro-2-methylpyridin-3-yl)oxy)-N-(3-(S-methyl-N-(methylglycyl)sulfonimidoyl)phenyl)-4-(trifluoromethyl)benzamide (68.9 mg, 67.2%) as a white solid. ¹H NMR (400 MHz, DMSO-d₆) δ 11.01 (s, 1H), 8.38 (s, 1H), 7.92-7.89 (m, 2H), 7.69-7.65 (m, 4H), 7.24 (s, 1H), 7.08-7.05 (m, 1H), 3.44 (s, 3H), 3.20 (s, 2H), 2.33 (s, 3H), 2.23 (s, 3H). LCMS (ESI) calcd. for C₂₄H₂₃F₄N₄O₄S [M+H]⁺ m/z 539.14, found 539.05.

### Example 11B

### (R)-2-((6-fluoro-2-methylpyridin-3-yl)oxy)-N-(3-(N-(2-hydroxyacetyl)-S-methylsulfonimidoyl)phenyl)-4-(trifluoromethyl)benzamide

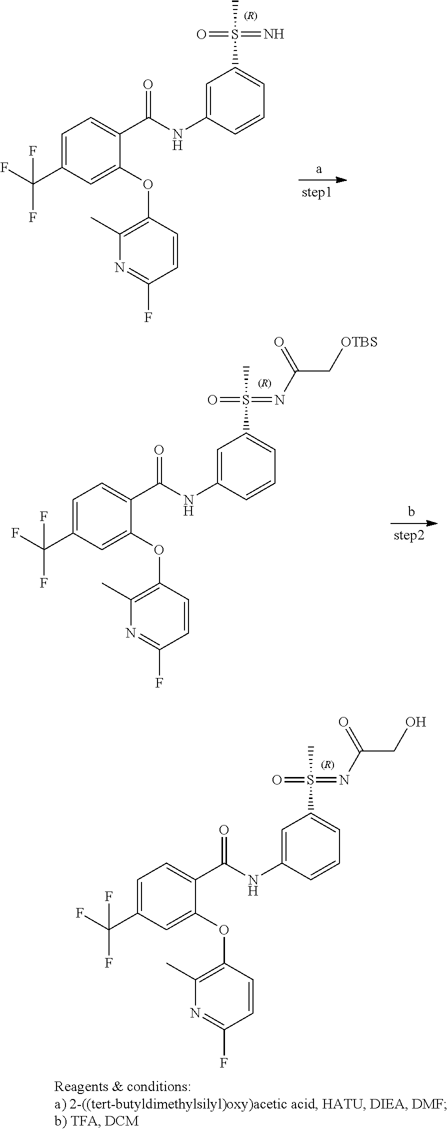

Step 1: A mixture of (R)-2-((6-fluoro-2-methylpyridin-3-yl)oxy)-N-(3-(S-methylsulfonimidoyl)phenyl)-4-(trifluoromethyl)benzamide (150 mg, 0.32 mmol) and 2-((tert-butyldimethylsilyl)oxy)acetic acid (105 mg, 0.55 mmol) in DMF (5 mL) was added HATU (158 mg, 0.42 mmol) and DIEA (133 mg, 1.02 mmol). The mixture was stirred at room temperature for 4 hours. The reaction was monitored by LCMS. After the reaction was completed, the resulting solution was diluted with water (20 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give (R)—N-(3-(N-(2-((tert-butyldimethylsilyl)oxy)acetyl)-S-methylsulfonimidoyl)phenyl)-2-((6-fluoro-2-methylpyridin-3-yl)oxy)-4-(trifluoromethyl)benzamide (100 mg, 49.02%) as a white oil. LCMS (ESI) calcd. for C₂₉H₃₄F₄N₃O₅SSi [M+H]⁺ m/z 640.19, found 640.05.

Step 2: A solution of (R)—N-(3-(N-(2-((tert-butyldimethylsilyl)oxy)acetyl)-S-methylsulfonimidoyl)phenyl)-2-((6-fluoro-2-methylpyridin-3-yl)oxy)-4-(trifluoromethyl)benzamide (100 mg, 0.16 mmol) in THF (3 mL) was added AcOH (3 mL) and H₂O (1 mL) at room temperature. The reaction mixture was stirred at room temperature for 2 hours. After the reaction was completed, the solution was diluted with water (20 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% formic acid) to obtain (R)-2-((6-fluoro-2-methylpyridin-3-yl)oxy)-N-(3-(N-(2-hydroxyacetyl)-S-methylsulfonimidoyl)phenyl)-4-(trifluoromethyl)benzamide (72.3 mg, 87%) as a white solid. ¹H NMR (400 MHz, DMSO-d₆) δ 11.03 (s, 1H), 8.36 (s, 1H), 7.93-7.90 (m, 2H), 7.69-7.63 (m, 4H), 7.23 (s, 1H), 7.08-7.05 (m, 1H), 4.80-4.78 (m, 1H), 3.94 (d, J=5.6 Hz, 2H), 3.44 (s, 3H), 2.33 (s, 3H). LCMS (ESI) calcd. for C₂₃H₂₀F₄N₃O₅S [M+H]⁺ m/z 526.11, found 526.00.

### Example 12B

### 2-((6-fluoro-2-methylpyridin-3-yl)oxy)-N-(3-((R)—N—((S)-2-hydroxypropanoyl)-S-methylsulfonimidoyl)phenyl)-4-(trifluoromethyl)benzamide

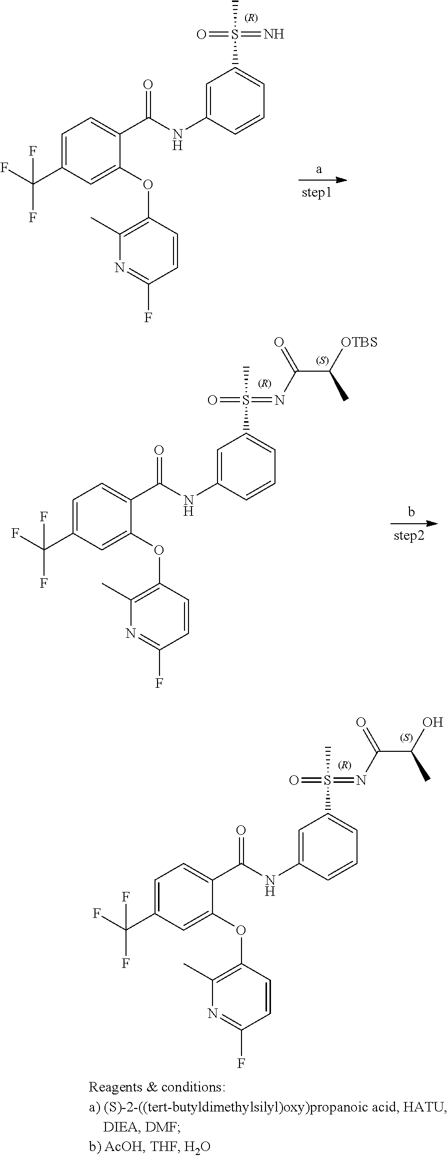

Step 1: A mixture of (R)-2-((6-fluoro-2-methylpyridin-3-yl)oxy)-N-(3-(S-2-methylsulfonimidoyl)phenyl)-4-(trifluoromethyl)benzamide (150 mg, 0.32 mmol) and (S)-2-((tert-butyldimethylsilyl)oxy)propanoic acid (112 mg, 0.55 mmol) in DMF (5 mL) was added HATU (158 mg, 0.42 mmol) and DIEA (133 mg, 1.02 mmol). The mixture was stirred at room temperature for 4 hours. The reaction was monitored by LCMS. After the reaction was completed, the resulting solution was diluted with water (40 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give N-(3-((R)—N—((S)-2-((tert-butyldimethylsilyl)oxy)propanoyl)-S-methylsulfonimidoyl)phenyl)-2-((6-fluoro-2-methylpyridin-3-yl)oxy)-4-(trifluoromethyl)benzamide (100 mg, 47.8%) as a white oil. LCMS (ESI) calcd. for C₃₀H₃₆F₄N₃O₅SSi [M+H]⁺ m/z 654.21, found 654.10.

Step 2: A solution of N-(3-((R)—N—((S)-2-((tert-butyldimethylsilyl)oxy)propanoyl)-S-methylsulfonimidoyl)phenyl)-2-((6-fluoro-2-methylpyridin-3-yl)oxy)-4-(trifluoromethyl)benzamide (100 mg, 0.15 mmol) in THF (3 mL) was added AcOH (3 mL) and H₂O (1 mL) at room temperature. The reaction mixture was stirred at room temperature for 2 hours. After the reaction was completed, the solution was diluted with water (30 mL) and extracted with DCM (20 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% formic acid) to obtain 2-((6-fluoro-2-methylpyridin-3-yl)oxy)-N-(3-((R)—N—((S)-2-hydroxypropanoyl)-S-methylsulfonimidoyl)phenyl)-4-(trifluoromethyl)benzamide (62.3 mg, 78%) as a white solid. ¹H NMR (400 MHz, DMSO-d₆) δ 11.02 (s, 1H), 8.39 (s, 1H), 7.91-7.87 (m, 2H), 7.69-7.65 (m, 4H), 7.23 (s, 1H), 7.08-7.05 (m, 1H), 4.68 (d, J=5.6 Hz, 1H), 4.04-4.01 (m, 1H), 3.43 (s, 3H), 2.33 (s, 3H), 1.24 (d, J=6.8 Hz, 3H). LCMS (ESI) calcd. for C₂₄H₂₂F₄N₃O₅S [M+H]⁺ m/z 540.12, found 540.05.

### Example 13B

### 2-((6-fluoro-2-methylpyridin-3-yl)oxy)-N-(3-((R)—N—((R)-2-hydroxypropanoyl)-S-methylsulfonimidoyl)phenyl)-4-(trifluoromethyl)benzamide

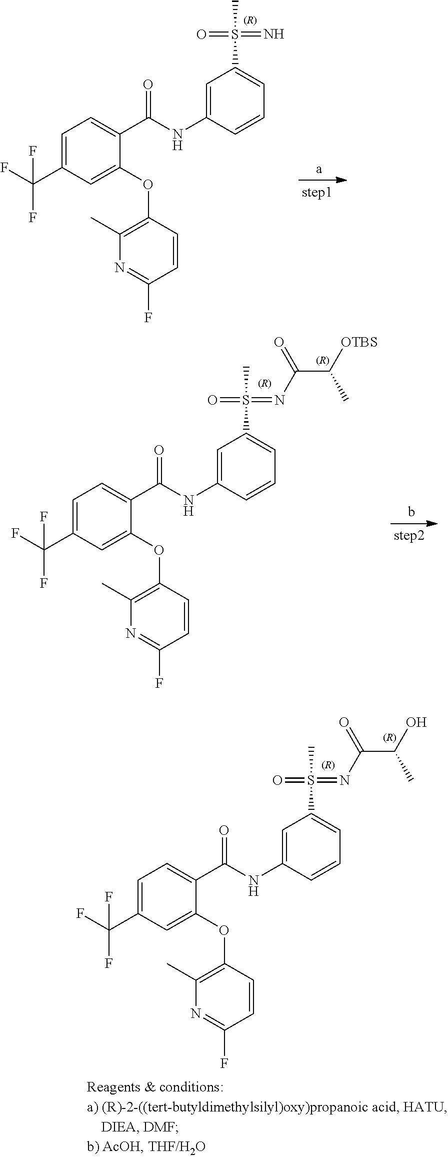

Step 1: N-(3-((R)—N—((R)-2-((tert-butyldimethylsilyl)oxy)propanoyl)-S-methylsulfonimidoyl)phenyl)-2-((6-fluoro-2-methylpyridin-3-yl)oxy)-4-(trifluoromethyl)benzamide: A mixture of (R)-2-((6-fluoro-2-methylpyridin-3-yl)oxy)-N-(3-(S-methylsulfonimidoyl)phenyl)-4-(trifluoromethyl)benzamide (300 mg, 0.64 mmol) and (R)-2-((tert-butyldimethylsilyl)oxy)propanoic acid (328 mg, 1.60 mmol) in DMF (4 mL) was added HATU (317 mg, 0.83 mmol) and DIEA (265 mg, 2.05 mmol). The mixture was stirred at room temperature for 2 hours. The reaction was monitored by LCMS. After the reaction was completed, the resulting solution was diluted with water (20 mL) and extracted with EtOAc (20 mL×3). The combined organic phases were washed with brine, dried over Na₂SO₄, concentrated under vacuum. The residue was purified by flash column chromatography on silica gel (PE/EtOAc=2/1 to 1/1) to give N-(3-((R)—N—((R)-2-((tert-butyldimethylsilyl)oxy)propanoyl)-S-methylsulfonimidoyl)phenyl)-2-((6-fluoro-2-methylpyridin-3-yl)oxy)-4-(trifluoromethyl)benzamide (200 mg, 47.7%) as a yellow oil. LCMS (ESI) calcd. for C₃₀H₃₆F₄N₃O₅SSi [M+H]⁺ m/z 654.20, found 654.10.

Step 2: 2-((6-fluoro-2-methylpyridin-3-yl)oxy)-N-(3-((R)—N—((R)-2-hydroxypropanoyl)-S-methylsulfonimidoyl)phenyl)-4-(trifluoromethyl)benzamide: A solution of N-(3-((R)—N—((R)-2-((tert-butyldimethylsilyl)oxy)propanoyl)-S-methylsulfonimidoyl)phenyl)-2-((6-fluoro-2-methylpyridin-3-yl)oxy)-4-(trifluoromethyl)benzamide (200 mg, 0.31 mmol) in THE (3 mL) was added CH₃CO₂H (3 mL) and H₂O (1 mL) at room temperature. The reaction mixture was stirred at room temperature for 5 hours. After the reaction was completed, the solution was diluted with water (10 mL) and extracted with EtOAc (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 90% McCN/H₂O containing 0.1% TFA) to obtain 2-((6-fluoro-2-methylpyridin-3-yl)oxy)-N-(3-((R)—N—((R)-2-hydroxypropanoyl)-S-methylsulfonimidoyl)phenyl)-4-(trifluoromethyl)benzamide (35 mg, 21.2%) as a white solid. ¹H NMR (400 MHz, CD₃OD) δ 8.41 (dd, J=4.0, 2.1 Hz, 1H), 7.91-7.86 (m, 2H), 7.78-7.75 (m, 1H), 7.67-7.60 (m, 2H), 7.53 (dd, J=8.7, 6.5 Hz, 1H), 7.18 (s, 1H), 6.92 (dd, J=8.6, 3.1 Hz, 1H), 4.21 (m, 1H), 3.43 (s, 3H), 2.39 (s, 3H), 1.40 (dd, J=6.9, 1.9 Hz, 3H). LCMS (ESI) calcd. for C₂₄H₂₂F₄N₃O₅S [M+H]⁺ m/z 540.11, found 540.10.

### Example 14B

### (R)-2-((6-fluoro-2-methylpyridin-3-yl)oxy)-N-(3-(N-glycyl-S-methylsulfonimidoyl)phenyl)-4-(trifluoromethyl)benzamide

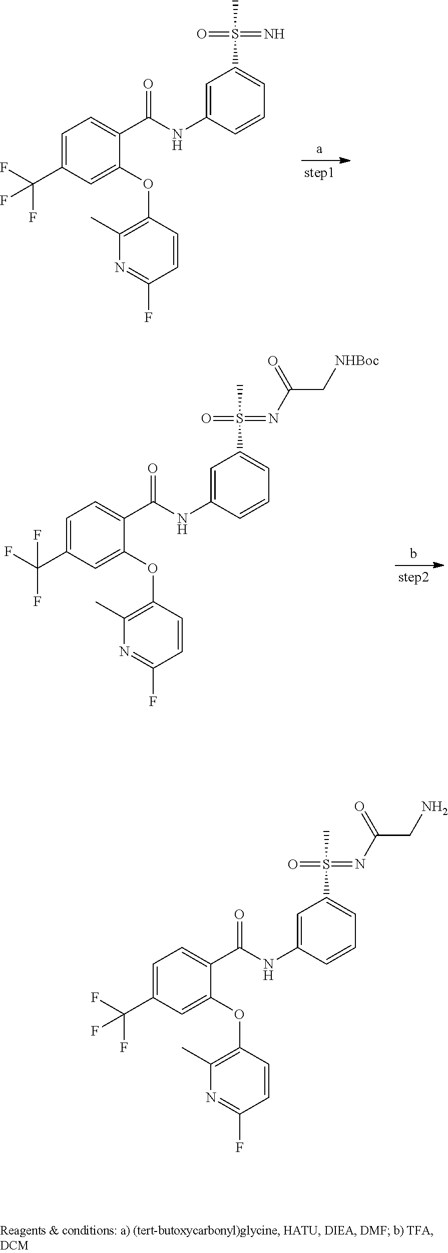 [SMILES: Cc1nc(F)ccc1Oc1cc(C(F)(F)F)ccc1C(=O)Nc1cccc([S@](C)(=N)=O)c1 ; Cc1nc(F)ccc1Oc1cc(C(F)(F)F)ccc1C(=O)Nc1cccc([S@@](C)(=O)=NC(=O)CNC(=O)OC(C)(C)C)c1 ; Cc1nc(F)ccc1Oc1cc(C(F)(F)F)ccc1C(=O)Nc1cccc([S@@](C)(=O)=NC(=O)CN)c1]

Step 1: tert-butyl (R)-(2-(((3-(2-((6-fluoro-2-methylpyridin-3-yl)oxy)-4-(trifluoromethyl)benzamido)phenyl)(methyl)(oxo)-λ⁶-sulfaneylidene)amino)-2-oxoethyl)carbamate: A mixture of (R)-2-((6-fluoro-2-methylpyridin-3-yl)oxy)-N-(3-(S-methylsulfonimidoyl)phenyl)-4-(trifluoromethyl)benzamide (150 mg, 0.32 mmol) and (tert-butoxycarbonyl)glycine (96 mg, 0.55 mmol) in DMF (2 mL) was added HATU (159 mg, 0.42 mmol) and DIEA (133 mg, 1.03 mmol). The mixture was stirred at room temperature for 2 hours. The reaction was monitored by LCMS. After the reaction was completed, the resulting solution was diluted with water (20 mL) and extracted with EtOAc (20 mL×3). The combined organic phases were washed with brine, dried over Na₂SO₄, concentrated under vacuum. The residue was purified by flash column chromatography on silica gel (PE/EtOAc=2/1 to 1/1) to give tert-butyl (R)-(2-(((3-(2-((6-fluoro-2-methylpyridin-3-yl)oxy)-4-(trifluoromethyl)benzamido)phenyl)(methyl)(oxo)-λ⁶-sulfaneylidene)amino)-2-oxoethyl)carbamate (150 mg, 74.9%) as a yellow oil. LCMS (ESI) calcd. for C₂₈H₂₉F₄N₄O₆S [M+H]⁺ m/z 625.17, found 625.05.

Step 2: (R)-2-((6-fluoro-2-methylpyridin-3-yl)oxy)-N-(3-(N-glycyl-S-methylsulfonimidoyl)phenyl)-4-(trifluoromethyl)benzamide: A solution of tert-butyl (R)-(2-(((3-(2-((6-fluoro-2-methylpyridin-3-yl)oxy)-4-(trifluoromethyl)benzamido)phenyl)(methyl)(oxo)-λ⁶-sulfaneylidene)amino)-2-oxoethyl)carbamate (150 mg, 0.24 mmol) in DCM (5 mL) was added TFA (0.5 mL) at room temperature. The reaction mixture was stirred at room temperature for 2 hours. After the reaction was completed, the solution was diluted with NaHCO₃ solution (10 mL) and extracted with EA (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 95% MeCN/H₂O containing 0.05% NH₃) to obtain (R)-2-((6-fluoro-2-methylpyridin-3-yl)oxy)-N-(3-(N-glycyl-S-methylsulfonimidoyl)phenyl)-4-(trifluoromethyl)benzamide (50 mg, 39.7%) as a white solid. ¹H NMR (400 MHz, DMSO-d₆) δ 11.01 (s, 1H), 8.36 (s, 1H), 7.91 (d, J=7.7 Hz, 2H), 7.69-7.62 (m, 4H), 7.24 (s, 1H), 7.07 (dd, J=8.7, 3.4 Hz, 1H), 3.44 (s, 3H), 3.24 (s, 2H), 2.33 (s, 3H), 1.99 (bs, 2H). LCMS (ESI) calcd. for C₂₃H₂₁F₄N₄O₄S [M+H]⁺ m/z 525.12, found 525.00.

### Example 15B

### N-(3-((R)—N-(D-alanyl)-S-methylsulfonimidoyl)phenyl)-2-fluoro-6-((6-fluoro-2-methylpyridin-3-yl)oxy)-3-(trifluoromethyl)benzamide

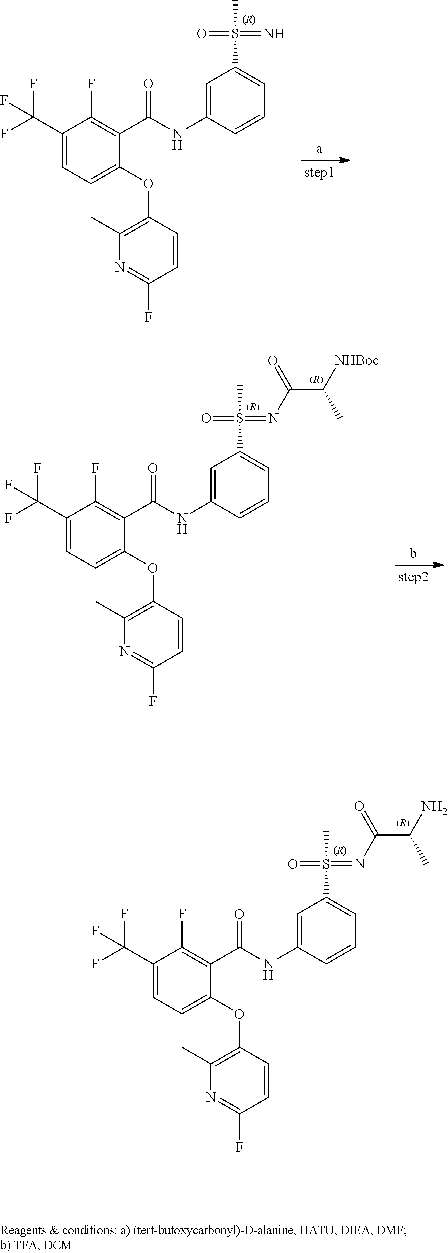

Step 1: tert-butyl ((R)-1-(((R)-(3-(2-fluoro-6-((6-fluoro-2-methylpyridin-3-yl)oxy)-3-(trifluoromethyl)benzamido)phenyl)(methyl)(oxo)-λ⁶-sulfaneylidene)amino)-1-oxopropan-2-yl)carbamate: A mixture of (R)-2-fluoro-6-((6-fluoro-2-methylpyridin-3-yl)oxy)-N-(3-(S-methylsulfonimidoyl)phenyl)-3-(trifluoromethyl)benzamide (100 mg, 0.21 mmol) and (tert-butoxycarbonyl)-D-alanine (78 mg, 0.41 mmol) in DMF (5 mL) was added HATU (117 mg, 0.31 mmol) and DIEA (80 mg, 0.62 mmol). The mixture was stirred at room temperature for 16 hours. The reaction was monitored by LCMS. After the reaction was completed, the resulting solution was diluted with water (20 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give tert-butyl ((R)-1-(((R)-(3-(2-fluoro-6-((6-fluoro-2-methylpyridin-3-yl)oxy)-3-(trifluoromethyl)benzamido)phenyl)(methyl)(oxo)-λ⁶-sulfaneylidene)amino)-1-oxopropan-2-yl)carbamate (80 mg, 59.1%) as a yellow solid. LCMS (ESI) calcd. for C₂₉H₃₀F₅N₄O₆S [M+H]⁺ m/z 657.18, found 657.00.

Step 2: N-(3-((R)—N-(D-alanyl)-S-methylsulfonimidoyl)phenyl)-2-fluoro-6-((6-fluoro-2-methylpyridin-3-yl)oxy)-3-(trifluoromethyl)benzamide: A solution of tert-butyl ((R)-1-(((R)-(3-(2-fluoro-6-((6-fluoro-2-methylpyridin-3-yl)oxy)-3-(trifluoromethyl)benzamido)phenyl)(methyl)(oxo)-λ⁶-sulfaneylidene)amino)-1-oxopropan-2-yl)carbamate (80 mg, 0.12 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (10 mL×3). The combined organic layers were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.05% NH₃) to obtain N-(3-((R)—N-(D-alanyl)-S-methylsulfonimidoyl)phenyl)-2-fluoro-6-((6-fluoro-2-methylpyridin-3-yl)oxy)-3-(trifluoromethyl)benzamide (30.5 mg, 44.3%) as a white solid. ¹H NMR (400 MHz, DMSO-d₆) δ 11.38 (s, 1H), 8.39 (s, 1H), 7.95-7.80 (m, 3H), 7.74-7.64 (m, 2H), 7.15 (dd, J=8.6, 3.2 Hz, 1H), 6.79 (d, J=8.9 Hz, 1H), 3.46 (s, 3H), 3.32-3.27 (m, 1H), 2.30 (s, 3H), 1.71 (s, 2H), 1.17 (d, J=6.9 Hz, 3H). LCMS (ESI) calcd. for C₂₄H₂₂F₅N₄O₄S [M+H]⁺ m/z 557.13, found 557.00.

### Example 16B

### (R)-2-fluoro-6-((6-fluoro-2-methylpyridin-3-yl)oxy)-N-(3-(N-glycyl-S-methylsulfonimidoyl)phenyl)-3-(trifluoromethyl)benzamide

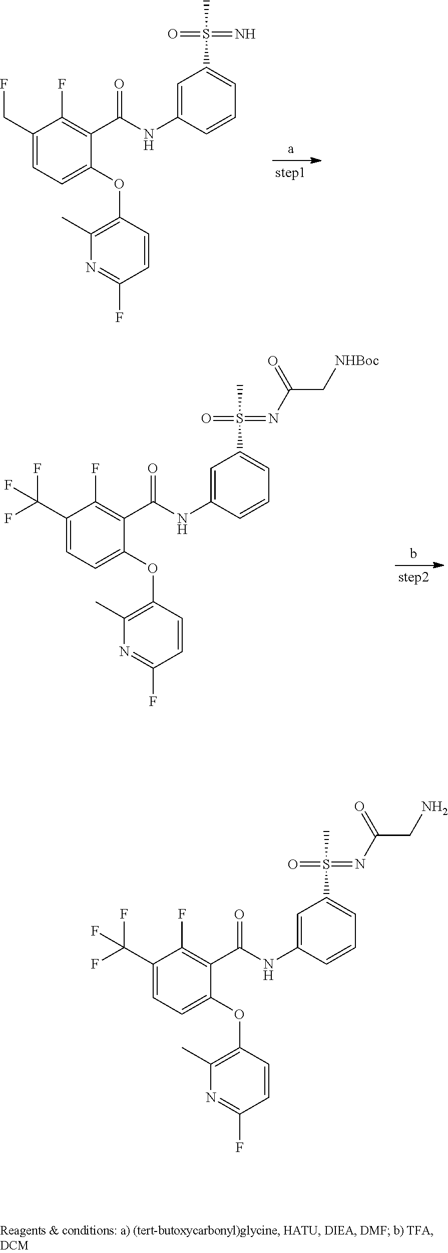 [SMILES: Cc1nc(F)ccc1Oc1ccc(CF)c(F)c1C(=O)Nc1cccc([S@](C)(=N)=O)c1 ; Cc1nc(F)ccc1Oc1ccc(C(F)(F)F)c(F)c1C(=O)Nc1cccc([S@@](C)(=O)=NC(=O)CNC(=O)OC(C)(C)C)c1 ; Cc1nc(F)ccc1Oc1ccc(C(F)(F)F)c(F)c1C(=O)Nc1cccc([S@@](C)(=O)=NC(=O)CN)c1]

Step 1: tert-butyl (R)-(2-(((3-(2-fluoro-6-((6-fluoro-2-methylpyridin-3-yl)oxy)-3-(trifluoromethyl)benzamido)phenyl)(methyl)(oxo)-λ⁶-sulfaneylidene)amino)-2-oxoethyl)carbamate: A mixture of (R)-2-fluoro-6-((6-fluoro-2-methylpyridin-3-yl)oxy)-N-(3-(S-methylsulfonimidoyl)phenyl)-3-(trifluoromethyl)benzamide (100 mg, 0.21 mmol) and (tert-butoxycarbonyl)glycine (72 mg, 0.41 mmol) in DMF (5 mL) was added HATU (117 mg, 0.31 mmol) and DIEA (80 mg, 0.62 mmol). The mixture was stirred at room temperature for 16 hours. The reaction was monitored by LCMS. After the reaction was completed, the resulting solution was diluted with water (20 mL) and extracted with DCM (20 mL×3). The combined organic layers were washed with brine, dried over Na₂SO₄, concentrated under vacuum. The residue was purified by flash column chromatography on silica gel (PE/EtOAc=2/1 to 1/1) to give tert-butyl (R)-(2-(((3-(2-fluoro-6-((6-fluoro-2-methylpyridin-3-yl)oxy)-3-(trifluoromethyl)benzamido)phenyl)(methyl)(oxo)-λ⁶-sulfaneylidene)amino)-2-oxoethyl)carbamate (80 mg, 60.4%) as a yellow solid. LCMS (ESI) calcd. for C₂₈H₂₈F₅N₄O₆S [M+H]⁺ m/z 643.16, found 643.00.

Step 2: (R)-2-fluoro-6-((6-fluoro-2-methylpyridin-3-yl)oxy)-N-(3-(N-glycyl-S-methylsulfonimidoyl)phenyl)-3-(trifluoromethyl)benzamide: A solution of tert-butyl (R)-(2-(((3-(2-fluoro-6-((6-fluoro-2-methylpyridin-3-yl)oxy)-3-(trifluoromethyl)benzamido)phenyl)(methyl)(oxo)-λ⁶-sulfaneylidene)amino)-2-oxoethyl)carbamate (80 mg, 0.12 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (10 mL×3). The combined organic layers were washed with brine, dried over sodium sulfate, and concentrated under vacuum. The residue was purified by prep-HPLC (Gemini 5 um C₁₈ column, 150*21.2 mm, eluting with 40% to 95% MeCN/H₂O containing 0.05% NH₃) to obtain (R)-2-fluoro-6-((6-fluoro-2-methylpyridin-3-yl)oxy)-N-(3-(N-glycyl-S-methylsulfonimidoyl)phenyl)-3-(trifluoromethyl)benzamide (22.5 mg, 32.9%) as a white solid. ¹H NMR (400 MHz, DMSO-d₆) δ 11.37 (s, 1H), 8.37 (s, 1H), 7.98-7.80 (m, 3H), 7.73-7.63 (m, 2H), 7.15 (dd, J=8.7, 3.3 Hz, 1H), 6.79 (d, J=8.9 Hz, 1H), 3.46 (s, 3H), 3.24 (s, 2H), 2.30 (s, 3H), 2.01 (s, 2H). LCMS (ESI) calcd. for C₂₃H₂₀F₅N₄O₄S [M+H]⁺ m/z 543.11, found 543.05.

### Example 17B

### (R)-2-fluoro-6-((6-fluoro-2-methylpyridin-3-yl)oxy)-N-(3-(S-methyl-N-(methylglycyl)sulfonimidoyl)phenyl)-3-(trifluoromethyl)benzamide

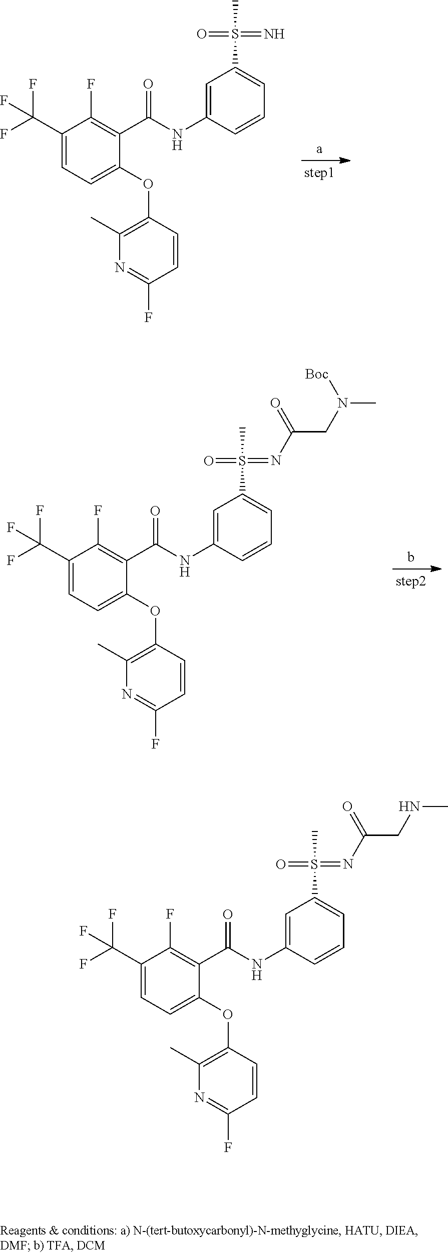 [SMILES: Cc1nc(F)ccc1Oc1ccc(C(F)(F)F)c(F)c1C(=O)Nc1cccc([S@](C)(=N)=O)c1 ; Cc1nc(F)ccc1Oc1ccc(C(F)(F)F)c(F)c1C(=O)Nc1cccc([S@@](C)(=O)=NC(=O)CN(C)C(=O)OC(C)(C)C)c1 ; CNCC(=O)N=[S@](C)(=O)c1cccc(NC(=O)c2c(Oc3ccc(F)nc3C)ccc(C(F)(F)F)c2F)c1]

Step 1: tert-butyl (R)-(2-(((3-(2-fluoro-6-((6-fluoro-2-methylpyridin-3-yl)oxy)-3-(trifluoromethyl)benzamido)phenyl)(methyl)(oxo)-λ⁶-sulfaneylidene)amino)-2-oxoethyl)(methyl)carbamate: A mixture of (R)-2-fluoro-6-((6-fluoro-2-methylpyridin-3-yl)oxy)-N-(3-(S-methylsulfonimidoyl)phenyl)-3-(trifluoromethyl)benzamide (100 mg, 0.21 mmol) and N-(tert-butoxycarbonyl)-N-methylglycine (78 mg, 0.41 mmol) in DMF (5 mL) was added HATU (117 mg, 0.31 mmol) and DIEA (80 mg, 0.62 mmol). The mixture was stirred at room temperature for 16 hours. The reaction was monitored by LCMS. After the reaction was completed, the resulting solution was diluted with water (20 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give tert-butyl (R)-(2-(((3-(2-fluoro-6-((6-fluoro-2-methylpyridin-3-yl)oxy)-3-(trifluoromethyl)benzamido)phenyl)(methyl)(oxo)-λ⁶-sulfaneylidene)amino)-2-oxoethyl)(methyl)carbamate (80 mg, 59.0%) as a yellow solid. LCMS (ESI) calcd. for C₂₉H₃₀F₅N₄O₆S [M+H]⁺ m/z 657.18, found 657.00.

Step 2: (R)-2-fluoro-6-((6-fluoro-2-methylpyridin-3-yl)oxy)-N-(3-(S-methyl-N-(methylglycyl)sulfonimidoyl)phenyl)-3-(trifluoromethyl)benzamide: A solution of tert-butyl (R)-(2-(((3-(2-fluoro-6-((6-fluoro-2-methylpyridin-3-yl)oxy)-3-(trifluoromethyl)benzamido)phenyl)(methyl)(oxo)-λ⁶-sulfaneylidene)amino)-2-oxoethyl)(methyl)carbamate (80 mg, 0.12 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (10 mL×3). The combined organic layers were washed with brine, dried over sodium sulfate, and concentrated under vacuum. The residue was purified by prep-HPLC (Gemini 5 um C₁₈ column, 150*21.2 mm, eluting with 40% to 95% MeCN/H₂O containing 0.05% NH₃) to obtain (R)-2-fluoro-6-((6-fluoro-2-methylpyridin-3-yl)oxy)-N-(3-(S-methyl-N-(methylglycyl)sulfonimidoyl)phenyl)-3-(trifluoromethyl)benzamide (41.3 mg, 60.1%) as a white solid. ¹H NMR (400 MHz, DMSO-d₆) δ 11.37 (s, 1H), 8.39 (s, 1H), 7.96-7.80 (m, 3H), 7.74-7.65 (m, 2H), 7.15 (dd, J=8.7, 3.4 Hz, 1H), 6.79 (d, J=8.8 Hz, 1H), 3.46 (s, 3H), 3.19 (s, 2H), 2.30 (s, 3H), 2.21 (s, 3H). LCMS (ESI) calcd. for C₂₄H₂₂F₅N₄O₄S [M+H]⁺ m/z 557.13, found 557.05.

### Example 18B

### 2-fluoro-6-((6-fluoro-2-methylpyridin-3-yl)oxy)-N-(3-((R)—N—((S)-2-hydroxypropanoyl)-S-methylsulfonimidoyl)phenyl)-3-(trifluoromethyl)benzamide

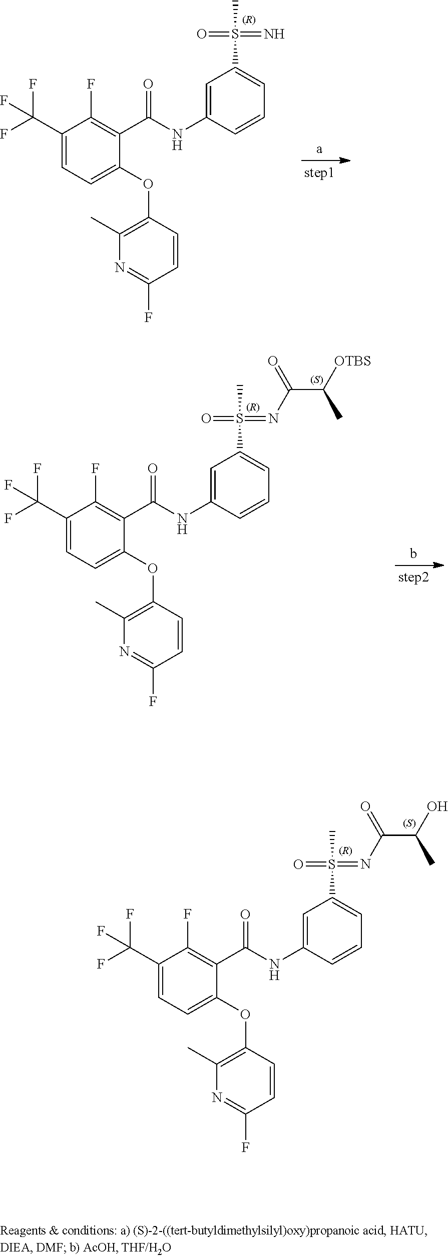

Step 1: N-(3-((R)—N—((S)-2-((tert-butyldimethylsilyl)oxy)propanoyl)-S-methylsulfonimidoyl)phenyl)-2-fluoro-6-((6-fluoro-2-methylpyridin-3-yl)oxy)-3-(trifluoromethyl)benzamide: A mixture of (R)-2-fluoro-6-((6-fluoro-2-methylpyridin-3-yl)oxy)-N-(3-(S-methylsulfonimidoyl)phenyl)-3-(trifluoromethyl)benzamide (150 mg, 0.31 mmol) and (S)-2-((tert-butyldimethylsilyl)oxy)propanoic acid (126 mg, 0.62 mmol) in DMF (5 mL) was added HATU (176 mg, 0.46 mmol) and DIEA (120 mg, 0.93 mmol). The mixture was stirred at room temperature for 16 hours. The reaction was monitored by LCMS. After the reaction was completed, the resulting solution was diluted with water (50 mL) and extracted with DCM (20 mL×3). The combined organic layers were washed with brine, dried over Na₂SO₄, concentrated under vacuum. The residue was purified by flash column chromatography on silica gel (PE/EtOAc=2/1 to 1/1) to give N-(3-((R)—N—((S)-2-((tert-butyldimethylsilyl)oxy)propanoyl)-S-methylsulfonimidoyl)phenyl)-2-fluoro-6-((6-fluoro-2-methylpyridin-3-yl)oxy)-3-(trifluoromethyl)benzamide (120 mg, 57.8%) as a yellow solid. LCMS (ESI) calcd. for C₃₀H₃₅F₅N₃O₅SSi [M+H]⁺ m/z 672.20, found 672.05.

Step 2: 2-fluoro-6-((6-fluoro-2-methylpyridin-3-yl)oxy)-N-(3-((R)—N—((S)-2-hydroxypropanoyl)-S-methylsulfonimidoyl)phenyl)-3-(trifluoromethyl)benzamide: A solution of N-(3-((R)—N—((S)-2-((tert-butyldimethylsilyl)oxy)propanoyl)-S-methylsulfonimidoyl)phenyl)-2-fluoro-6-((6-fluoro-2-methylpyridin-3-yl)oxy)-3-(trifluoromethyl)benzamide (120 mg, 0.18 mmol) in THE (3 mL) and H₂O (1 mL) was added AcOH (3 mL) at room temperature. The reaction mixture was stirred at room temperature for 2 hours. After the reaction was completed, the solution was diluted with water (30 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 95% MeCN/H₂O containing 0.1% formic acid) to obtain 2-fluoro-6-((6-fluoro-2-methylpyridin-3-yl)oxy)-N-(3-((R)—N—((S)-2-hydroxypropanoyl)-S-methylsulfonimidoyl)phenyl)-3-(trifluoromethyl)benzamide (41.2 mg, 41.2%) as a white solid. ¹H NMR (400 MHz, DMSO-d₆) δ 11.37 (s, 1H), 8.40 (s, 1H), 7.97-7.78 (m, 3H), 7.74-7.65 (m, 2H), 7.15 (dd, J=8.7, 3.4 Hz, 1H), 6.79 (d, J=8.9 Hz, 1H), 4.70 (d, J=5.6 Hz, 1H), 4.10-3.97 (m, 1H), 3.45 (s, 3H), 2.30 (s, 3H), 1.24 (d, J=6.8 Hz, 3H).

LCMS (ESI) calcd. for C₂₄H₂₁F₅N₃O₅S [M+H]⁺ m/z 558.11, found 558.00.

### Example 19B

### 2-fluoro-6-((6-fluoro-2-methylpyridin-3-yl)oxy)-N-(3-((R)—N—((R)-2-hydroxypropanoyl)-S-methylsulfonimidoyl)phenyl)-3-(trifluoromethyl)benzamide

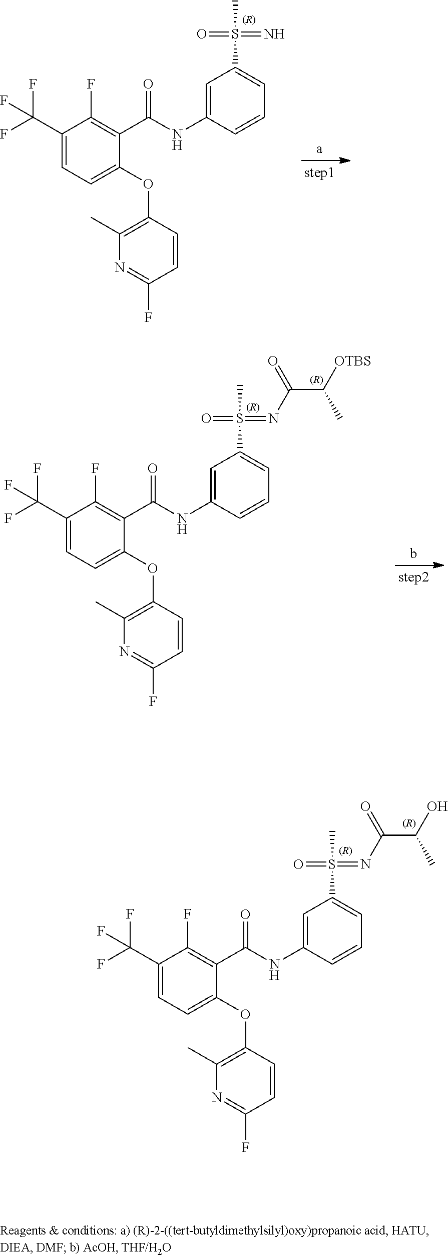

Step 1: N-(3-((R)—N—((R)-2-((tert-butyldimethylsilyl)oxy)propanoyl)-S-methylsulfonimidoyl)phenyl)-2-fluoro-6-((6-fluoro-2-methylpyridin-3-yl)oxy)-3-(trifluoromethyl)benzamide: A mixture of (R)-2-fluoro-6-((6-fluoro-2-methylpyridin-3-yl)oxy)-N-(3-(S-methylsulfonimidoyl)phenyl)-3-(trifluoromethyl)benzamide (150 mg, 0.31 mmol) and (R)-2-((tert-butyldimethylsilyl)oxy)propanoic acid (126 mg, 0.62 mmol) in DMF (5 mL) was added HATU (176 mg, 0.46 mmol) and DIEA (120 mg, 0.93 mmol). The mixture was stirred at room temperature for 16 hours. The reaction was monitored by LCMS. After the reaction was completed, the resulting solution was diluted with water (50 mL) and extracted with DCM (30 mL×3). The combined organic layers were washed with brine, dried over Na₂SO₄, concentrated under vacuum. The residue was purified by flash column chromatography on silica gel (PE/EtOAc=2/1 to 1/1) to give N-(3-((R)—N—((R)-2-((tert-butyldimethylsilyl)oxy)propanoyl)-S-methylsulfonimidoyl)phenyl)-2-fluoro-6-((6-fluoro-2-methylpyridin-3-yl)oxy)-3-(trifluoromethyl)benzamide (120 mg, 57.8%) as a yellow solid. LCMS (ESI) calcd. for C₃₀H₃₅F₅N₃O₅SSi [M+H]⁺ m/z 672.20, found 672.05.

Step 2: 2-fluoro-6-((6-fluoro-2-methylpyridin-3-yl)oxy)-N-(3-((R)—N—((R)-2-hydroxypropanoyl)-S-methylsulfonimidoyl)phenyl)-3-(trifluoromethyl)benzamide. A mixture of N-(3-((R)—N—((R)-2-((tert-butyldimethylsilyl)oxy)propanoyl)-S-methylsulfonimidoyl)phenyl)-2-fluoro-6-((6-fluoro-2-methylpyridin-3-yl)oxy)-3-(trifluoromethyl)benzamide (120 mg, 0.18 mmol) in THE (3 mL) and H₂O (1 mL) was added AcOH (3 mL) at room temperature. The reaction mixture was stirred at room temperature for 2 hours. After the reaction was completed, the solution was diluted with water (30 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 95% MeCN/H₂O containing 0.1% formic acid) to obtain 2-fluoro-6-((6-fluoro-2-methylpyridin-3-yl)oxy)-N-(3-((R)—N—((R)-2-hydroxypropanoyl)-S-methylsulfonimidoyl)phenyl)-3-(trifluoromethyl)benzamide (40.5 mg, 40.6%) as a white solid. ¹H NMR (400 MHz, DMSO-d₆) δ 11.37 (s, 1H), 8.39 (s, 1H), 7.97-7.78 (m, 3H), 7.76-7.64 (m, 2H), 7.15 (dd, J=8.6, 3.4 Hz, 1H), 6.79 (d, J=8.8 Hz, 1H), 4.73 (d, J=5.3 Hz, 1H), 4.10-3.95 (m, 1H), 3.48 (s, 3H), 2.30 (s, 3H), 1.27 (d, J=6.8 Hz, 3H). LCMS (ESI) calcd. for C₂₄H₂₁F₅N₃O₅S [M+H]⁺ m/z 558.11, found 558.00.

### Example 20B

### (R)-2-fluoro-6-((6-fluoro-2-methylpyridin-3-yl)oxy)-N-(3-(N-(2-hydroxyacetyl)-S-methylsulfonimidoyl)phenyl)-3-(trifluoromethyl)benzamide

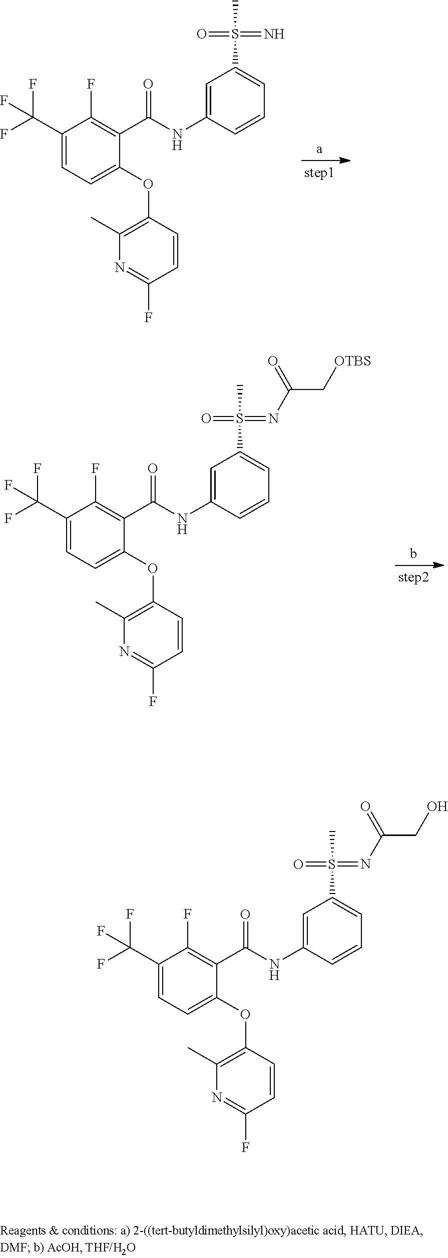 [SMILES: Cc1nc(F)ccc1Oc1ccc(C(F)(F)F)c(F)c1C(=O)Nc1cccc([S@](C)(=N)=O)c1 ; Cc1nc(F)ccc1Oc1ccc(C(F)(F)F)c(F)c1C(=O)Nc1cccc([S@@](C)(=O)=NC(=O)CO[Si](C)(C)C(C)(C)C)c1 ; Cc1nc(F)ccc1Oc1ccc(C(F)(F)F)c(F)c1C(=O)Nc1cccc([S@@](C)(=O)=NC(=O)CO)c1]

Step 1: (R)—N-(3-(N-(2-((tert-butyldimethylsilyl)oxy)acetyl)-S-methylsulfonimidoyl)phenyl)-2-fluoro-6-((6-fluoro-2-methylpyridin-3-yl)oxy)-3-(trifluoromethyl)benzamide: A mixture of (R)-2-fluoro-6-((6-fluoro-2-methylpyridin-3-yl)oxy)-N-(3-(S-methylsulfonimidoyl)phenyl)-3-(trifluoromethyl)benzamide (150 mg, 0.309 mmol) and 2-((tert-butyldimethylsilyl)oxy)acetic acid (117.61 mg, 0.618 mmol) in DMF (5 mL) was added HATU (176.24 mg, 0.464 mmol) and DIEA (119.81 mg, 0.927 mmol). The mixture was stirred at room temperature for 8 hours. The reaction was monitored by LCMS. After the reaction was completed, the resulting solution was diluted with water (50 mL) and extracted with DCM (30 mL×3). The combined organic layers were washed with brine, dried over Na₂SO₄, concentrated under vacuum. The residue was purified by flash column chromatography on silica gel (PE/EtOAc=2/1 to 1/1) to give (R)—N-(3-(N-(2-((tert-butyldimethylsilyl)oxy)acetyl)-S-methylsulfonimidoyl)phenyl)-2-fluoro-6-((6-fluoro-2-methylpyridin-3-yl)oxy)-3-(trifluoromethyl)benzamide (120 mg, 53.14%) as a yellow solid. LCMS (ESI) calcd. for C₂₉H₃₃F₅N₃O₅SSi [M+H]⁺ m/z 658.19, found 658.10.

Step 2: (R)-2-fluoro-6-((6-fluoro-2-methylpyridin-3-yl)oxy)-N-(3-(N-(2-hydroxyacetyl)-S-methylsulfonimidoyl)phenyl)-3-(trifluoromethyl)benzamide: A solution of (R)—N-(3-(N-(2-((tert-butyldimethylsilyl)oxy)acetyl)-S-methylsulfonimidoyl)phenyl)-2-fluoro-6-((6-fluoro-2-methylpyridin-3-yl)oxy)-3-(trifluoromethyl)benzamide (120 mg, 0.1824 mmol) in THF (1 mL) and H₂O (3 mL) was added AcOH (3 mL) at room temperature. The reaction mixture was stirred at room temperature for 6 hours. The residue was adjusted to pH=8-9 with saturated aqueous NaHCO₃. Then the aqueous solution was extracted with EtOAc (20 mL×3). The combined organic layers were washed with brine, dried over sodium sulfate, and concentrated under vacuum. The residue was purified by prep-HPLC (Gemini 5 um C₁₈ column, 150*21.2 mm, eluting with 40% to 50% to 95% MeCN/H₂O containing 0.1% FA) to afford (R)-2-fluoro-6-((6-fluoro-2-methylpyridin-3-yl)oxy)-N-(3-(N-(2-hydroxyacetyl)-S-methylsulfonimidoyl)phenyl)-3-(trifluoromethyl)benzamide (27.2 mg, 26.32%) as a white solid. ¹H NMR (400 MHz, DMSO-d₆) δ 11.39 (s, 1H), 8.36 (s, 1H), 7.98-7.80 (m, 3H), 7.74-7.65 (m, 2H), 7.15 (dd, J=8.7, 3.3 Hz, 1H), 6.79 (d, J=8.9 Hz, 1H), 4.79 (t, J=6.2 Hz, 1H), 3.94 (d, J=5.9 Hz, 2H), 3.47 (s, 3H), 2.30 (s, 3H). LCMS (ESI) calcd. C₂₃H₁₉F₅N₃O₅S [M+H]⁺ m/z 544.10, found 543.80.

### Example 21B

### (S)—N—((R)-(3-(2-fluoro-6-((6-fluoro-2-methylpyridin-3-yl)oxy)-3-(trifluoromethyl)benzamido)phenyl)(methyl)(oxo)-)⁶-sulfaneylidene)pyrrolidine-2-carboxamide

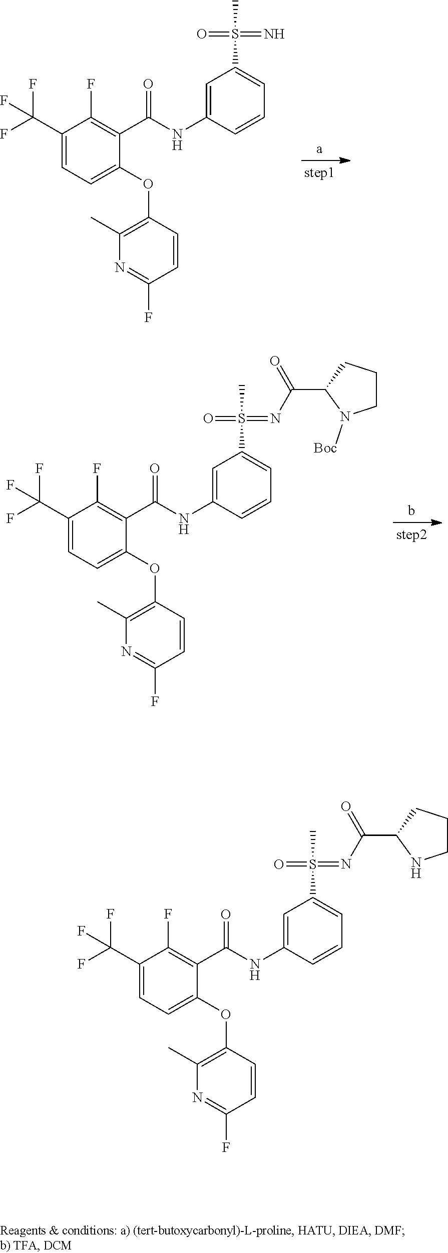 [SMILES: Cc1nc(F)ccc1Oc1ccc(C(F)(F)F)c(F)c1C(=O)Nc1cccc([S@](C)(=N)=O)c1 ; Cc1nc(F)ccc1Oc1ccc(C(F)(F)F)c(F)c1C(=O)Nc1cccc([S@@](C)(=O)=NC(=O)[C@@H]2CCCN2C(=O)OC(C)(C)C)c1 ; Cc1nc(F)ccc1Oc1ccc(C(F)(F)F)c(F)c1C(=O)Nc1cccc([S@@](C)(=O)=NC(=O)[C@@H]2CCCN2)c1]

Step 1: tert-butyl (S)-2-(((R)-(3-(2-fluoro-6-((6-fluoro-2-methylpyridin-3-yl)oxy)-3-(trifluoromethyl)benzamido)phenyl)(methyl)(oxo)-λ⁶-sulfaneylidene)carbamoyl)pyrrolidine-1-carboxylate: A mixture of (R)-2-fluoro-6-((6-fluoro-2-methylpyridin-3-yl)oxy)-N-(3-(S-methylsulfonimidoyl)phenyl)-3-(trifluoromethyl)benzamide (150 mg, 0.31 mmol) and (tert-butoxycarbonyl)-L-proline (133.39 mg, 0.62 mmol) in DMF (5 mL) was added HATU (175.89 mg, 0.46 mmol) and DIEA (119.57 mg, 0.93 mmol). The mixture was stirred at room temperature for 3 hours. The reaction was monitored by LCMS. After the reaction was completed, the resulting solution was diluted with water (40 mL) and extracted with DCM (20 mL×3). The combined organic layers were washed with brine, dried over Na₂SO₄, concentrated under vacuum. The residue was purified by flash column chromatography on silica gel (PE/EtOAc=2/1 to 1/1) to give tert-butyl (S)-2-(((R)-(3-(2-fluoro-6-((6-fluoro-2-methylpyridin-3-yl)oxy)-3-(trifluoromethyl)benzamido)phenyl)(methyl)(oxo)-λ⁶-sulfaneylidene)carbamoyl)pyrrolidine-1-carboxylate (120 mg, 51.23%) as a yellow solid. LCMS (ESI) calcd. for C₃₁H₃₂F₅N₄O₆S [M+H]⁺ m/z 683.2, found 681.05.

Step 2: (S)—N—((R)-(3-(2-fluoro-6-((6-fluoro-2-methylpyridin-3-yl)oxy)-3-(trifluoromethyl)benzamido)phenyl)(methyl)(oxo)-λ⁶-sulfaneylidene)pyrrolidine-2-carboxamide: A solution of tert-butyl (S)-2-(((R)-(3-(2-fluoro-6-((6-fluoro-2-methylpyridin-3-yl)oxy)-3-(trifluoromethyl)benzamido)phenyl)(methyl)(oxo)-λ⁶-sulfaneylidene) carbamoyl)pyrrolidine-1-carboxylate (120 mg, 0.17 mmol) in DCM (10 mL) was added TFA (1 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 50% to 60 to 95% MeCN/H₂O containing 0.05% NH₃) to obtain (S)—N—((R)-(3-(2-fluoro-6-((6-fluoro-2-methylpyridin-3-yl)oxy)-3-(trifluoromethyl)benzamido)phenyl)(methyl)(oxo)-λ⁶-sulfaneylidene)pyrrolidine-2-carboxamide (41.3 mg, 38.8%) as a white solid. ¹H NMR (400 MHz, DMSO-d₆) δ11.38 (s, 1H), 8.42 (s, 1H), 7.89-7.67 (m, 5H), 7.15 (dd, J=8, 7, 3.4 Hz, 1H), 6.79 (d, J=8.8 Hz, 1H), 3.51 (dd, J=8.5, 5.8 Hz, 1H), 3.45 (s, 3H), 2.90-2.83 (m, 1H), 2.61 (dt, J=10.0, 6.8 Hz, 1H), 2.30 (s, 3H), 2.02-1.93 (m, 1H), 1.79-1.71 (m, 1H), 1.61-1.43 (m, 2H). LCMS (ESI) calcd. for C₂₆H₂₄F₅N₄O₄S [M+H]⁺ m/z 583.15, found 582.90.

The following compounds are prepared using the techniques of the intermediates and examples described above.

| | | MS | |
|---|---|---|---|
| | | (m/z, | |
| Ex. No | Structure | M + H)⁺ | NMR data |
| 22B | 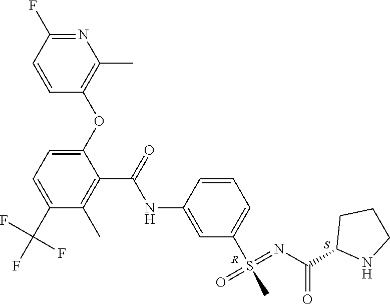 | 578.9 | ¹H NMR (400 MHz, DMSO- d₆, ppm) δ 11.24 (s, 1 H), 8.51 (d, J = 1.6 Hz, 1 H), 8.27 (d, J = 2.5 Hz, 1 H), 7.90- 7.64 (m, 5 H), 7.12 (dd, J = 8.6, 3.2 Hz, 1 H), 6.74 (d, J = 8.8 Hz, 1 H), 3.75-3.71 (m, 1 H), 3.48 (s, 3 H), 2.98-2.88 (m, 1 H), 2.81-2.71 (m, 1 H), 2.45 (s, 3 H), 2.28 (s, 3 H), 2.11-2.00 (m, 1 H), 1.89- 1.79 (m, 1 H), 1.72-1.48 (m, 2 H). |
| 23B | 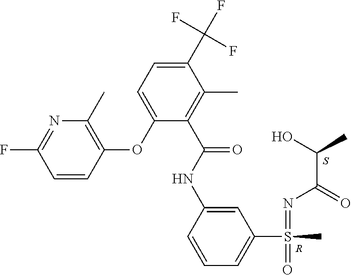 | 553.9 | ¹H NMR (400 MHz, DMSO- d₆, ppm) δ 11.24 (s, 1 H), 8.51 (s, 1 H), 8.01-7.90 (m, 1 H), 7.86-7.65 (m, 4 H), 7.16 (dd, J = 8.7, 3.4 Hz, 1 H), 6.78 (d, J = 8.8 Hz, 1 H), 4.74 (d, J = 5.6 Hz, 1 H), 4.14- 4.00 (m, 1 H), 3.50 (s, 3 H), 2.50 (s, 3 H), 2.32 (s, 3 H), 1.29 (d, J = 8.0 Hz, 3 H). |
| 24B | 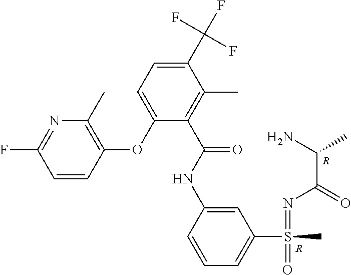 | 553.0 | ¹H NMR (400 MHz, DMSO- d₆, ppm) δ 11.17 (s, 1 H), 8.45 (s, 1 H), 7.94-7.89 (m, 1 H), 7.80-7.70 (m, 2 H), 7.69- 7.63 (m, 2 H), 7.14-7.10 (m, 1 H), 6.74 (d, J = 8.8 Hz, 1 H), 3.43 (s, 3 H), 3.29 (s, 1 H), 2.46 (s, 3 H), 2.28 (s, 3 H), 1.15 (d, J = 6.9 Hz, 3 H). |
| 25B | 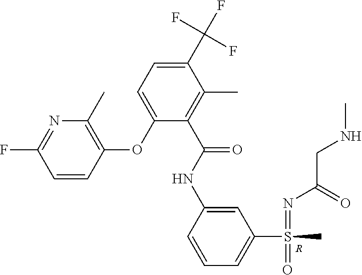 | 553.1 | ¹H NMR (400 MHz, DMSO- d₆, ppm) δ 11.17 (s, 1 H), 8.44 (s, 1 H), 7.97-7.91 (m, 1 H), 7.79-7.64 (m, 4 H), 7.12 (dd, J = 8.7, 3.4 Hz, 1 H), 6.74 (d, J = 8.8 Hz, 1 H), 3.45 (s, 3 H), 3.33-3.26 (m, 2 H), 2.46 (s, 3 H), 2.28 (s, 3 H), 1.27-1.22 (m, 1 H), 1.17 (d, J = 6.9 Hz, 3 H). |
| 26B | 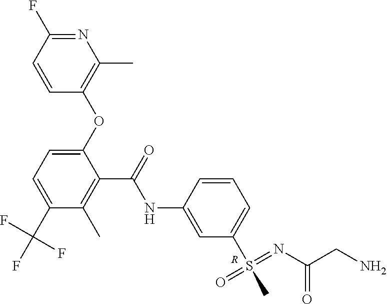 | 539.1 | ¹H NMR (400 MHz, DMSO- d₆, ppm) δ 11.17 (s, 1 H), 8.42 (s, 1 H), 8.00-7.92 (m, 1 H), 7.82-7.61 (m, 4 H), 7.12 (dd, J = 8.7, 3.4 Hz, 1 H), 6.75 (d, J = 8.8 Hz, 1 H), 3.45 (s, 3 H), 3.23 (s, 2 H), 2.46 (s, 3 H), 2.29 (s, 3 H). |
| 27B | 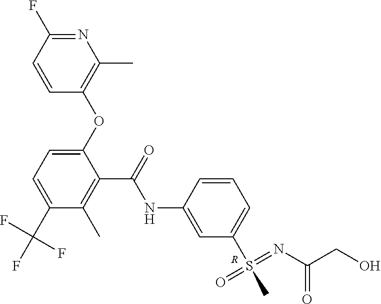 | 540.1 | ¹H NMR (400 MHz, DMSO- d₆, ppm) δ 11.18 (s, 1 H), 8.42 (d, J = 1.8 Hz, 1 H), 7.96 (dd, J = 7.2, 2.1 Hz, 1 H), 7.81-7.63 (m, 4 H), 7.12 (dd, J = 8.7, 3.5 Hz, 1 H), 6.74 (d, J = 8.8 Hz, 1 H), 4.78 (t, J = 6.3 Hz, 1 H), 3.94 (d, J = 6.3 Hz, 2 H), 3.46 (s, 3 H), 2.46 (s, 3 H), 2.28 (s, 3 H). |
| 28B | 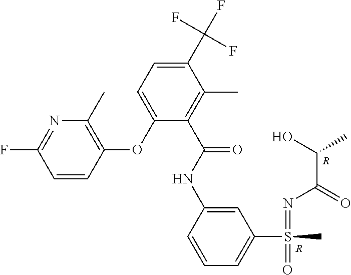 | 554.0 | ¹H NMR (400 MHz, DMSO-d₆, ppm) δ 11.18 (s, 1 H), 8.45 (s, 1 H), 7.98- 7.92 (m, 1 H), 7.80-7.63 (m, 4 H), 7.12 (dd, J = 8.7, 3.4 Hz, 1 H), 6.74 (d, J = 8.8 Hz, 1 H), 4.71 (d, J = 5.3 Hz, 1 H), 4.07-3.97 (m, 1 H), 3.47 (s, 3 H), 2.46 (s, 3 H), 2.28 (s, 3 H), 1.26 (d, J = 6.8 Hz, 3 H). |
| 29B | 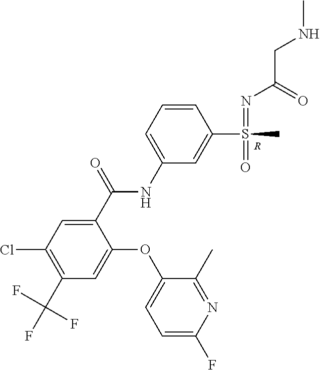 | 573.0 | ¹H NMR (400 MHz, DMSO- d₆, ppm) δ 11.06 (s, 1 H), 8.33 (s, 1 H), 8.12 (s, 1 H), 7.89 (d, J = 7.6 Hz, 1 H), 7.67 (dt, J = 11.1, 7.4 Hz, 3 H), 7.36 (s, 1 H), 7.05 (dd, J = 8.7, 3.4 Hz, 1 H), 3.44 (s, 3 H), 3.19 (s, 2 H), 2.34 (s, 3 H), 2.22 (s, 3 H). |

### Example 30B

### Compound Profiling Human NaV1.8/β3 Cell Line—SyncroPatch384PE Assay

Compounds were tested on recombinant human Nav1.8/b3 stably transfected CHO cells using the SyncroPatch384PE system (Nanion Technologies), an automated patch clamp device. Cells were cultured at 37° C./5% CO₂ in Ham's F-12 supplemented with 10% fetal bovine serum, 100 U/mL penicillin G sodium, 100 mg/mL streptomycin sulfate and selection antibiotics (0.01 mg/ml Blasticidin, 0.4 mg/ml Zeocin and 0.25 mg/ml Hygromycin). On the day of the recordings, cells in culture dishes were washed twice with Hank's Balanced Salt Solution (HBSS) and treated with Accutase for approximately 20 minutes. Immediately before use in the SynchroPatch384PE using an 8-hole NPC-384 chip, the cells were washed in HBSS to remove the Accutase and resuspended in extracellular solution. All experiments were performed at ambient temperature. Intracellular solution contained (mM): CsCl, 50; CsF, 90; MgCl₂, 5; EGTA, 1; HEPES, 10; pH adjusted to 7.2 with CsOH. Extracellular solution contained (mM): NaCl, 137; KCl, 4.0; CaCl₂), 3.8; MgCl₂, 1; HEPES, 10; Glucose, 10; pH adjusted to 7.4 with NaOH. 100 nM tetrodotoxin (TTX) was added to the extracellular solution to block endogenous TTX-sensitive sodium currents.

Compounds were tested in quadruplicate in 0.3% DMSO and 0.03% pluronic Acid. Compounds were diluted 1:3.33 in extracellular solution to create an 8-point concentration response curve. Each plate contained a historical positive control and up to ten compounds. 300 μM tetracaine and 0.3% DMSO+0.03% pluronic acid were used as high and low controls respectively.

Whole cell patch clamp recordings were conducted according to Nanion's standard procedure for SyncroPatch384PE®. Cells were held at a holding potential of −120 mV. A depolarization step to 10 mV for 30 ms was applied (P1 measurement), followed by a hyperpolarization step to −100 mV for 100 ms. An inactivation step at −35 mV for 10 sec was applied before stepping to −100 mV for 20 ms, followed by a step to 10 mV for 30 ms (P2 measurement) and then back to −100 mV for 30 ms. Sweep interval was 15 sec. Following establishment of the whole-cell configuration in extracellular solution, cells were washed in extracellular solution containing 0.3% DMSO and 0.03% pluronic acid to stabilize the baseline current. Compounds were then applied by the SynchroPatch384 PE system into each well and the current was recorded for five minutes in extracellular solution, followed by application of tetracaine to achieve full block at the end of experiment. The potency of the compounds was assessed on two read-outs, resting state block (P1 measurement) or inactivated state block (P2 measurement) to obtain IC50 values. Values were normalized to high (tetracaine) and low (DMSO+pluronic acid) controls.

The table below shows the potency of compounds against human Nav1.8, where “A” represents an IC50 less than or equal to 5 nM, “B” represents an IC50 greater than 5 nM to less than or equal to 50 nM, “C” represents an IC50 greater than 50 nM to less than or equal to 100 nM, “D” represents an IC50 greater than 100 nM to less than or equal to 200 nM, “E” represents an IC50 greater than 200 nM.

| Example | P1 | P2 |
|---|---|---|
| 1B | A | A |
| 2B | A | A |
| 3B | A | A |
| 4B | A | A |
| 5B | A | A |
| 6B | A | A |
| 7B | A | A |
| 8B | A | A |
| 9B | A | A |
| 10B | B | B |
| 11B | B | B |
| 12B | B | B |
| 13B | B | B |
| 14B | B | B |
| 15B | A | A |
| 16B | A | A |
| 17B | A | A |
| 18B | A | A |
| 19B | A | A |
| 21B | B | B |
| 22B | A | A |
| 23B | A | A |
| 24B | A | A |
| 25B | A | A |
| 26B | A | A |
| 27B | A | A |
| 28B | A | A |
| 29B | A | A |

### (C) Third Set of Compounds

In one aspect, the invention provides a compound of Formula (I):

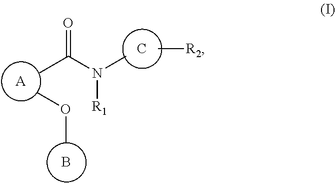

and pharmaceutically acceptable salts, hydrates and solvates thereof, wherein:
A is a substituted or unsubstituted heteroaryl ring comprising at least one heteroatom selected from a group consisting of O, S, or N;
wherein the one or more substitutions on A are selected from H, —OH, halo, C₁-C₈-alkyl, C₁-C₈ fully or partially fluorinated fluoroalkyl, C₂-C₈ branched alkyl, alkenyl, alkynyl, haloalkyl, alkoxy, cycloalkoxy, haloalkoxy, nitro, cyano, —C(R′)(R″)-cycloalkyl, C(R′)(R″)-aryl, —NR′R″, substituted or unsubstituted 3-8 membered cycloalkyl, 3-8 membered cycloalkenyl, or 3-6 membered heterocycloalkyl, wherein the 3-6 membered heterocycloalkyl comprises at least one heteroatom independently selected from O, S, and N;
B is substituted or unsubstituted aryl or heteroaryl, wherein the substitutions are selected from the group consisting of substituted or unsubstituted C₁-C₈ alkyl, deuterated C₁-C₄ alkyl wherein the alkyl chain may be fully or partially deuterated, halo-C₁-C₄ alkyl where the alkyl chain is fully or partially halogenated, C₃-C₁₀ cycloalkyl, halogen, cyano, nitro, C₁-C₈ alkoxyl, haloalkoxyl wherein the haloalkyoxyl chain may be fully or partially halogenated, or arylalkoxyl;
C is substituted or unsubstituted aryl or heteroaryl, wherein the one or more substitutions are independently selected from a group consisting of halo, C₁-C₈ alkyl, haloalkyl, alkoxy;
R₁ is selected from a group consisting of: H and C₁-C₄ alkyl;
R₂ is selected from a group consisting of: Formula (II):

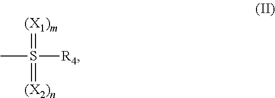

—C(═O)NH₂, and —C(═O)NR′R″;
   wherein,
m and n are independently 0 or 1; and
X₁ is O and X₂ is either NH or NR′; or
X₁ and X₂ are both O, NH, or NR′; or
X₁ is O and X₂ is NR₃; wherein R₃ is selected from a group consisting of: CD₃, C₁-C₄ alkyl or cycloalkyl, haloalkyl, halocycloalkyl, aryl, heteroaryl, and heterocyclyl, (C₁-C₆)alkyl-OH, (C₁-C₆)alkyl-NHR′, (C₁-C₆)alkyl-NR′R″, (C₁-C₆)alkyl-O—(C₁-C₆)alkyl, (C₁-C₆)alkyl-N—(C₁-C₆)alkyl;
R₄ is selected from a group consisting of: —NH₂, —NR′R″, C₁-C₄ alkyl, C₃-C₈-cycloalkyl, haloalkyl, halocycloalkyl, aryl, heteroaryl, and heterocyclyl.

In another embodiment, B is a 6 membered substituted or unsubstituted heteroaryl ring, comprising one or more N atoms. The N atoms in the heteroaryl ring may be in the form of a N-oxide. In certain embodiments, the N-oxide containing B-ring is selected from pyridyl N-oxide, pyrazinyl N-oxide, and pyrimidinyl N-oxide.

In another embodiment, the compound is a compound of Formula (III):

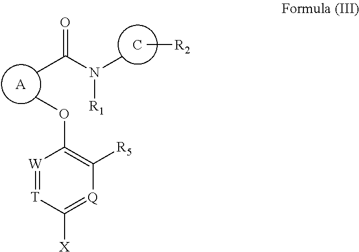

wherein, Q, T and W are independently N or CR₆;
R₆ is H, halogen, —CD₃, alkyl, cycloalkyl, haloalkyl, alkoxy, haloalkoxy, cyano, —CF₃, —OCF₃, or substituted or unsubstituted cycloalkoxy;
R₅ is H, —OH, halo, —CD₃, C₁-C₆-alkyl, branched alkyl, haloalkyl where the alkyl chain is fully or partially halogenated, alkoxy, arylalkoxy, cycloalkoxy, haloalkoxy, cyano, —CH₂-cycloalkyl, —CH(CH₃)-cycloalkyl, trifluoromethyl, cyclopropylmethyl, substituted or unsubstituted 3-6 membered cycloalkyl, any of which may have one or more substituents;
X is H, halo, —CD₃, alkyl, haloalkyl, alkoxy, cycloalkoxy, haloalkoxy, cyano, —CF₃, —OCF₃.

In another embodiment, the compound is a compound of Formula (IV):

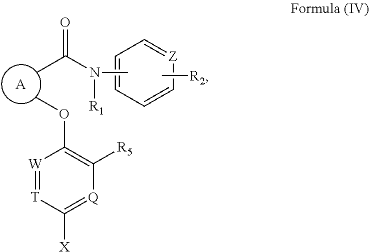

wherein:
Z is CR₁₀, N, or NO; wherein R₁₀ is H, halo, —CD₃, C₁-C₈ alkyl, haloalkyl, or alkoxy.

In certain embodiments, ring A is substituted or unsubstituted 6 or more membered heteroaryl ring having at least one heteroatom independently selected from N, O, or S.

In certain embodiments, ring A is substituted or unsubstituted pyridyl, pyrimidinyl, pyrazinyl, or pyridazinyl.

In certain embodiments, ring A is substituted or unsubstituted 6 membered heteroaryl having at least one heteroatom, wherein the heteroatom is N. In certain embodiments, ring A is substituted or unsubstituted 6 membered heteroaryl having at least 2 N atoms.

In other embodiments, in the compounds of Formula (I), Formula (III), and/or Formula (IV), A is:

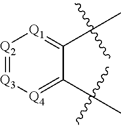

wherein Q₁, Q₂, Q₃ and Q₄ are independently selected from a group consisting of: N, N⁺O⁻, or CR₇; wherein at least two of Q₁, Q₂, Q₃ and Q₄ are CR₇;
R₇ is H, —OH, halo, —CD₃, alkyl, haloalkyl, alkoxy, haloalkoxy, cyano, —CF₃, —OCF₃, substituted or unsubstituted 5 or 6 membered ring heterocyclyl or heteroaryl, saturated heterocyclyl, or partially unsaturated heterocyclyl, O-aryl, O-heteroaryl, O-cycloalkyl, or O-cycloheteroalkyl.

In other embodiments, in the compounds of Formula (I), Formula (III), and/or Formula (IV), A is:

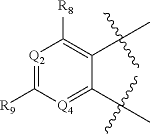

wherein,
Q₂ and Q₄ are independently N or N⁺O⁻;
Q₂ is N or N⁺O⁻; Q₄ is CR₇; or
Q₂ is CR₇, Q₄ is N or N⁺O⁻;
R₈ and R₉ are independently selected from a group consisting of H, —OH, halo, —CD₃, substituted or unsubstituted C₁-C₆ alkyl, branched alkyl, alkenyl, alkylnyl, haloalkyl, alkoxy, cycloalkyl, heterocyclyl, NH₂, NHR′, NR′R″, aryl, heteroaryl, —CF₂CH₃, and —CF₂CF₃;

In other embodiments, in the compounds of Formula (I), Formula (III), and/or Formula (IV), A is:

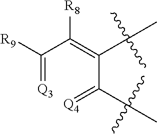

wherein,
Q₃ and Q₄ are independently N or N⁺O⁻;
Q₃ is N or N⁺O⁻ and Q₄ is CR₇; or
Q₃ is CR₇, Q₄ is N or N⁺O⁻; wherein R₇, R₈ and R₉ are defined above.

In other embodiments, in the compounds of Formula (I), Formula (III), and/or Formula (IV), A is:

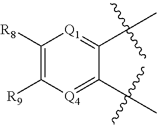

wherein,
Q₁ and Q₄ are independently N or N⁺O⁻;
Q₁ is N or N⁺O⁻, and Q₄ is CR₇;
Q₁ is CR₇ and Q₄ is N or N⁺O⁻; or
Q₁ or Q₄ is CR₇; wherein R₇, R₈, and R₉ are defined above.

In other embodiments, in the compounds of Formula (I), Formula (III), and/or Formula (IV), A is:

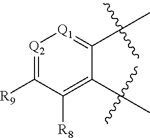

wherein Q₁ and Q₂ are N; wherein R₈ and R₉ are defined above.

In other embodiments, in the compounds of Formula (I), Formula (III), and/or Formula (IV), A is:

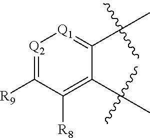

wherein Q₁ is CR₇, Q₂ is N; R₇, R₈, and R₉ are defined above.

In other embodiments, in the compounds of Formula (I), Formula (III), and/or Formula (IV), A is:

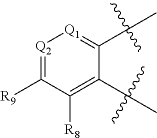

wherein, Q₁ is N; Q₂ is CR₇; R₇, R₈, and R₉ are defined above.

In other embodiments, in the compounds of Formula (I), Formula (III), and/or Formula (IV), R₂ is:

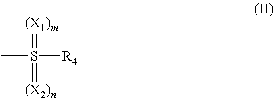

wherein m and n are independently 0 or 1;
X₁ is O and X₂ is NH, and R₄ is alkyl, for example, methyl.

In other embodiments, in the compounds of Formula (I), Formula (III), and/or Formula (IV), R₂ is:

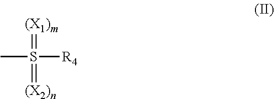

wherein m and n are independently 0 or 1;
X₁ is O and X₂ is O, and R₄ is NH₂ or alkyl, for example, methyl.

In other embodiments, R₂ is —S(═O)CH₃. In another embodiments, in the compound of Formula (III) and/or Formula (IV), Q is N or CH; R₅ is methyl or —OMe, X is F or CN. In other embodiments, in the compound of Formula (III) and/or Formula (IV), Q is CF. In other embodiments, R₇, R₈, and R₉ are independently selected from a group consisting of: H, methyl, fluoro, chloro, bromo, CF₃, cyclopropyl, difluorophenyl, and dimethylpyrazole. In other embodiments, in the compound of Formula (III) and/or Formula (IV), R₁₀ is H or F. In other embodiments, in the compound of Formula (III) and/or Formula (IV), Z is N or CH.

In certain embodiments of the invention, R₂ does not comprise —C(═O)NH₂ or —C(═O)NR′R″.

In certain embodiments, R₂ is Formula (II):

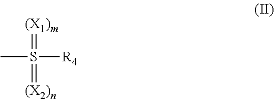

wherein,
m and n are independently 0 or 1; and
X₁ and X₂ are not both O, NH, or NR′.

In other embodiments, the compound of the invention is selected from a group consisting of:

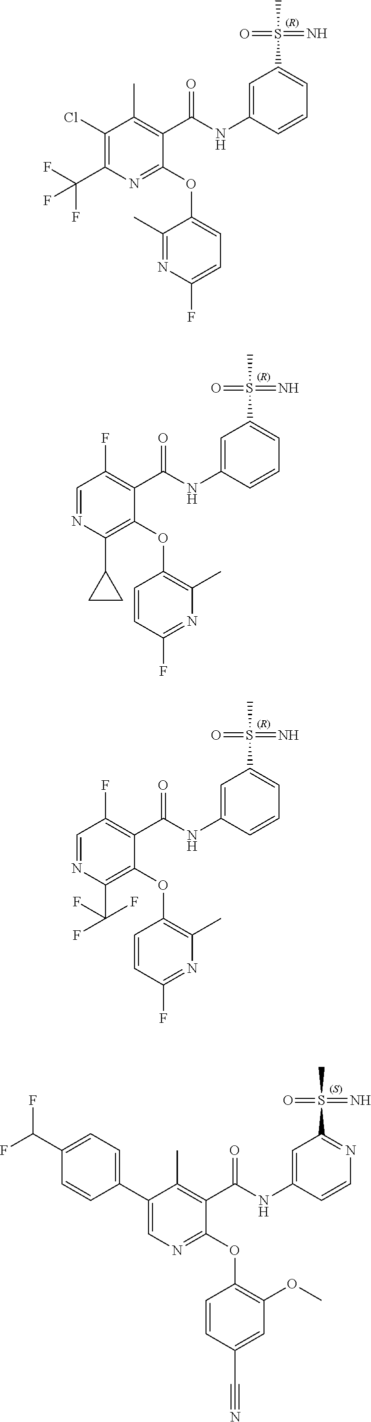

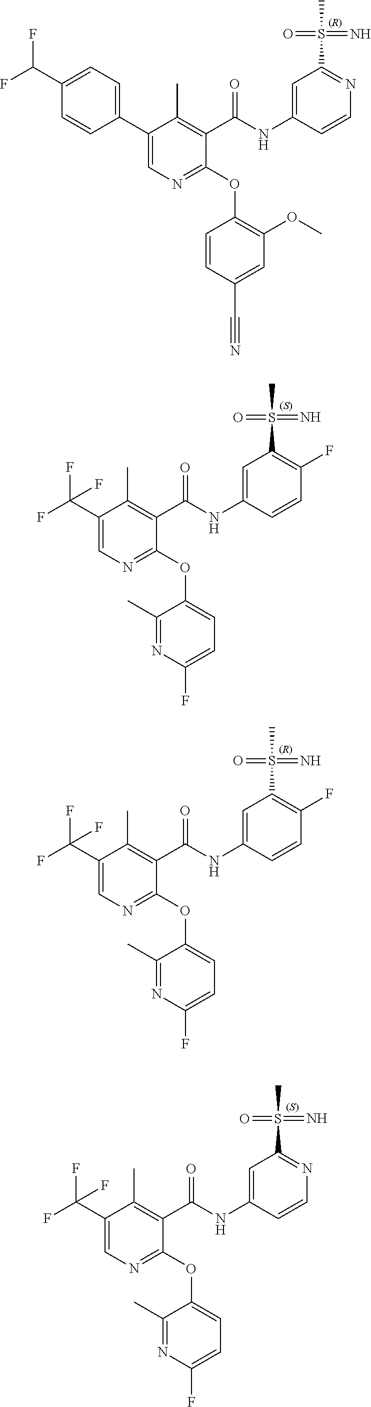

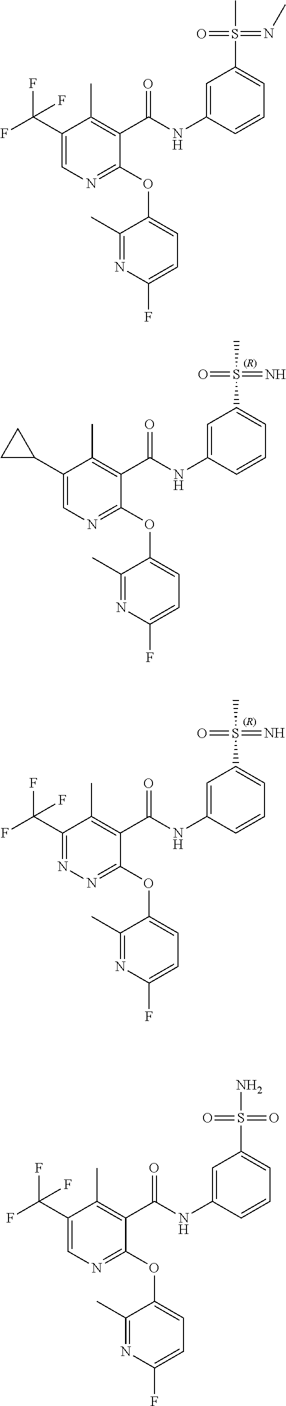

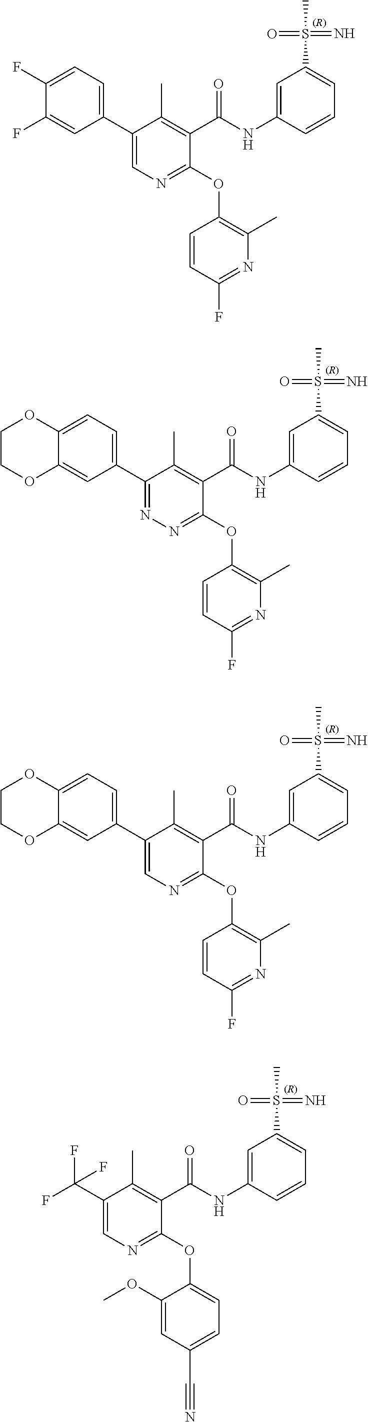

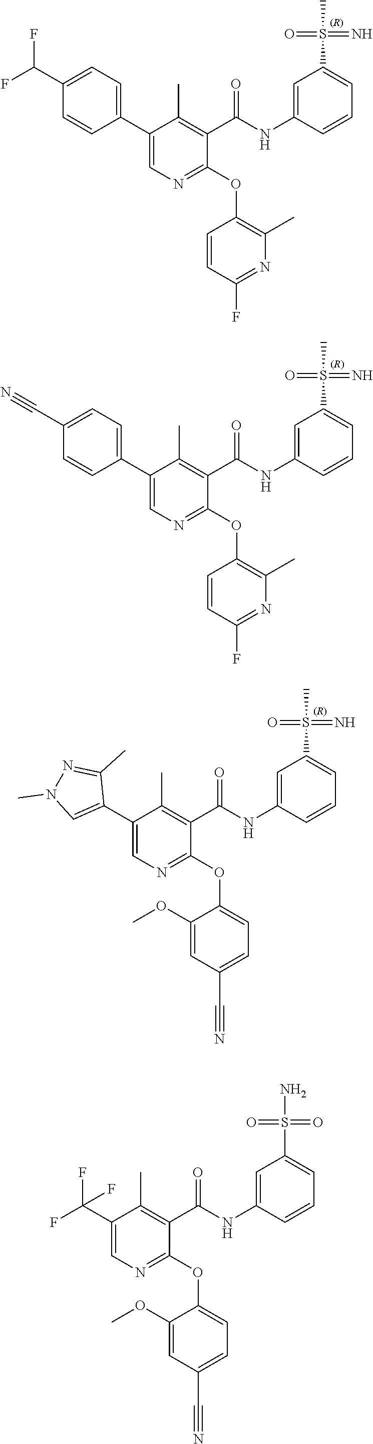

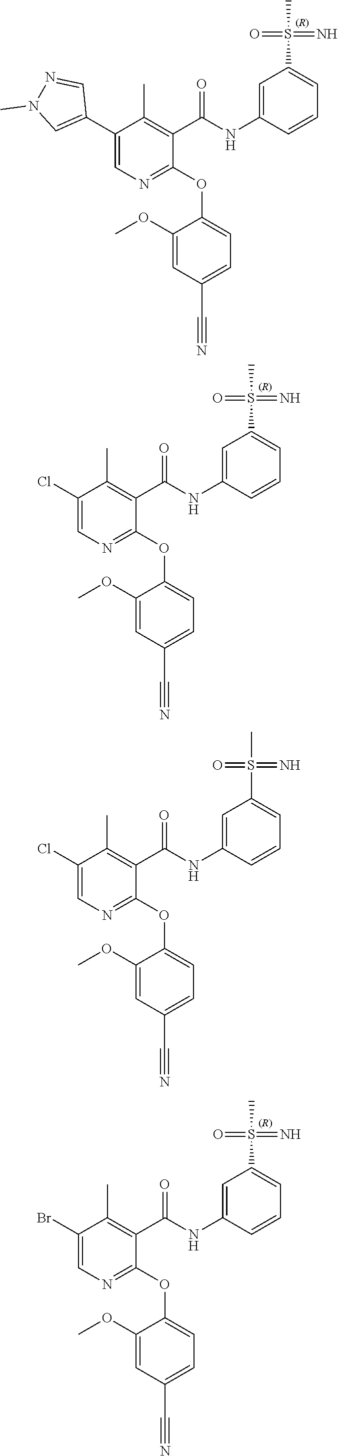

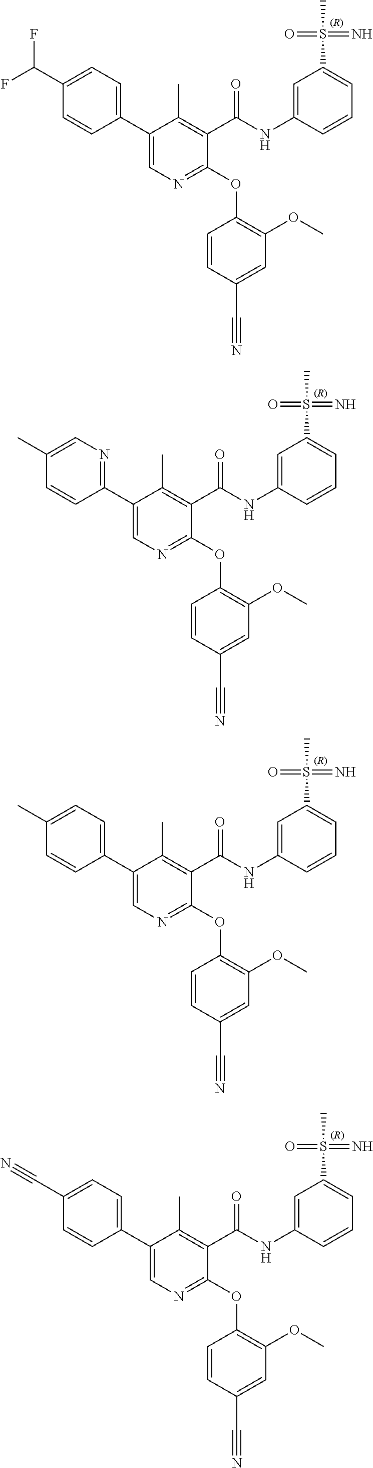

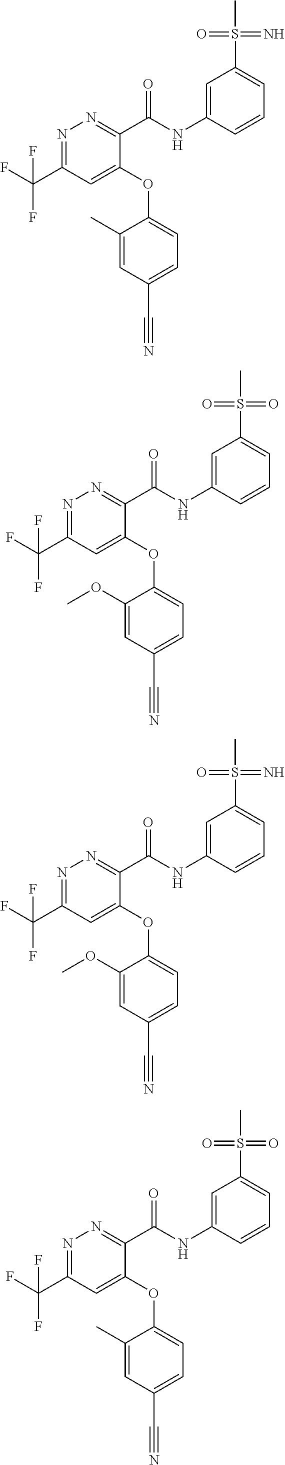 [SMILES: Cc1cc(C#N)ccc1Oc1cc(C(F)(F)F)nnc1C(=O)Nc1cccc(S(C)(=N)=O)c1 ; COc1cc(C#N)ccc1Oc1cc(C(F)(F)F)nnc1C(=O)Nc1cccc(S(C)(=O)=O)c1 ; COc1cc(C#N)ccc1Oc1cc(C(F)(F)F)nnc1C(=O)Nc1cccc(S(C)(=N)=O)c1 ; Cc1cc(C#N)ccc1Oc1cc(C(F)(F)F)nnc1C(=O)Nc1cccc(S(C)(=O)=O)c1]

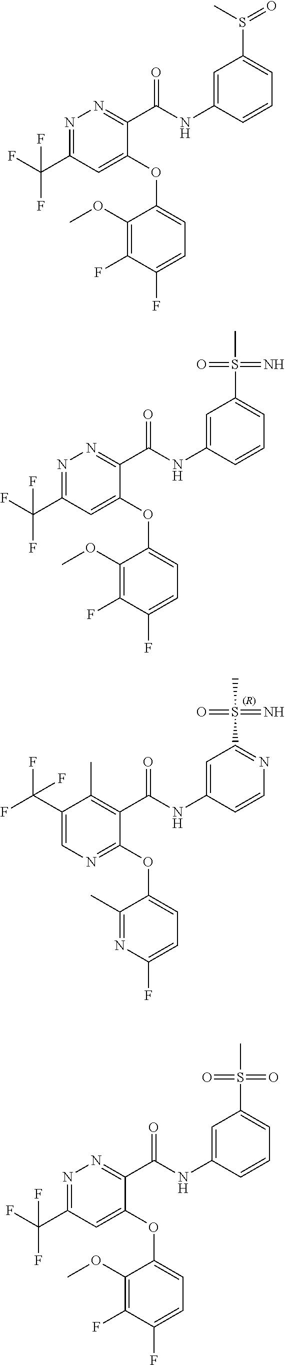

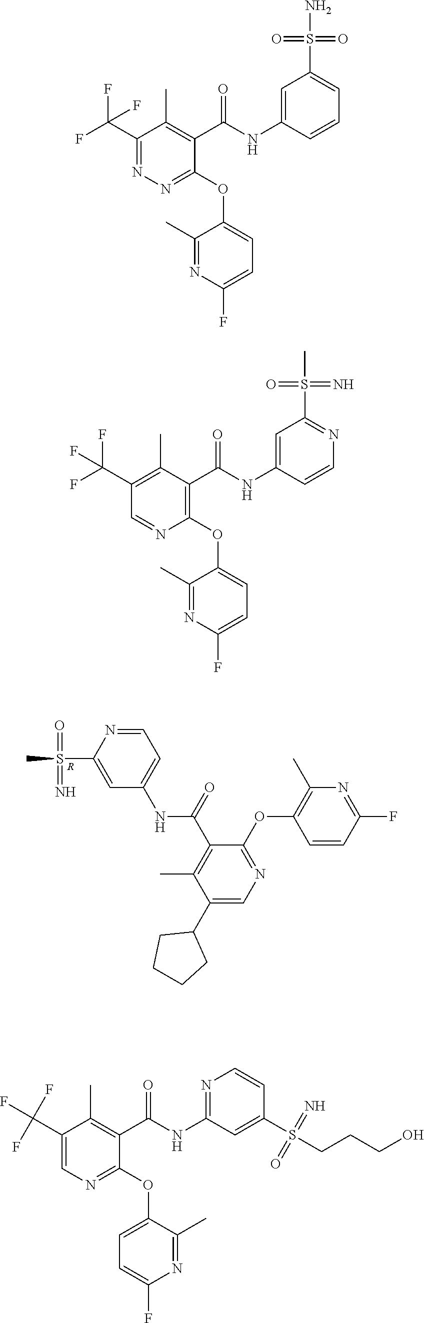

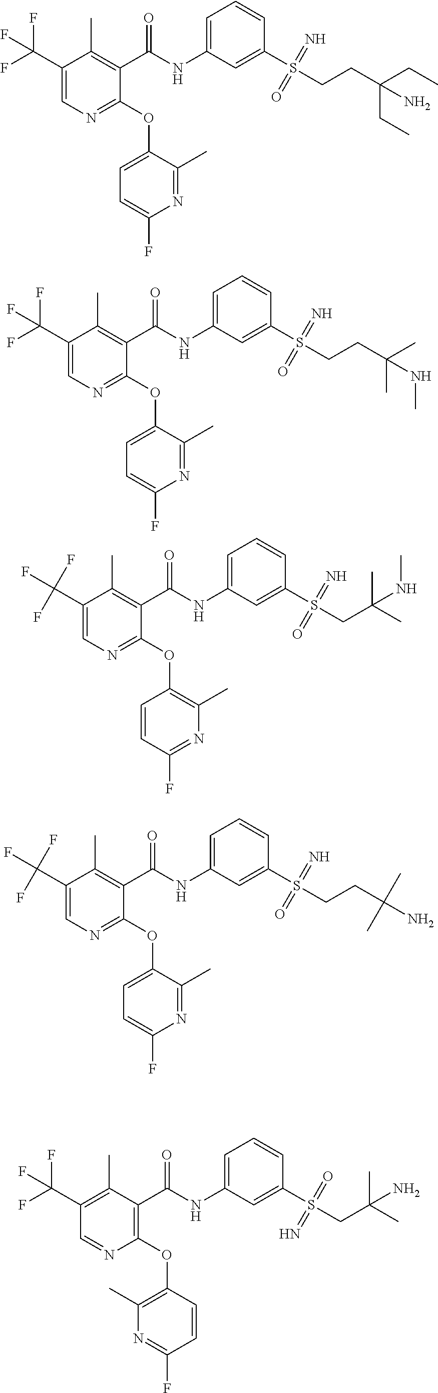

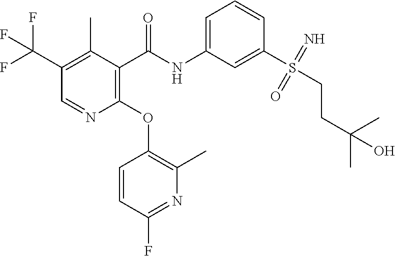 [SMILES: Cc1nc(F)ccc1Oc1ncc(C(F)(F)F)c(C)c1C(=O)Nc1cccc(S(=N)(=O)CCC(C)(C)O)c1]

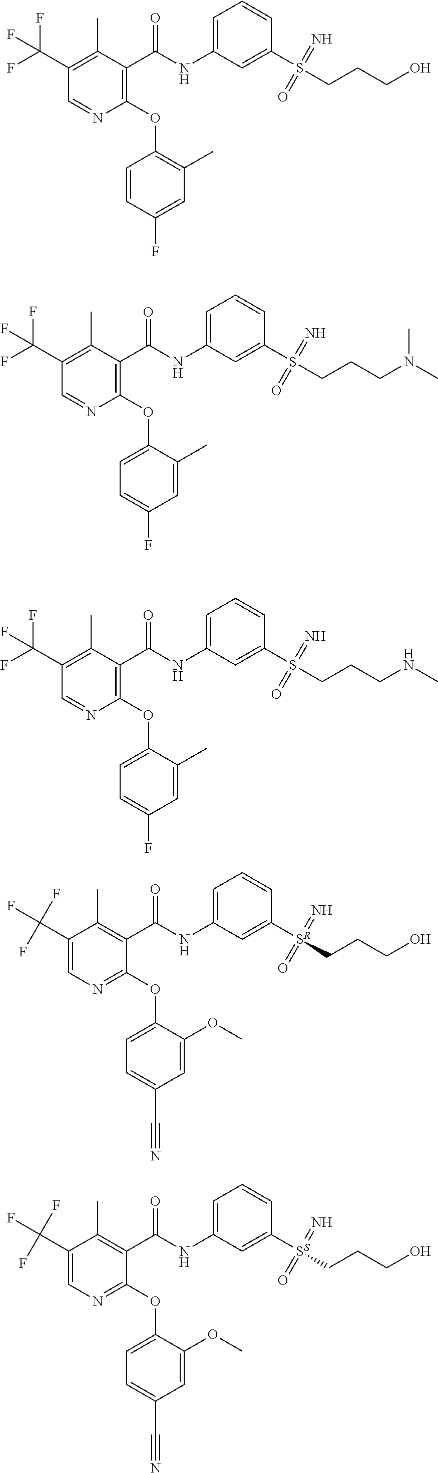

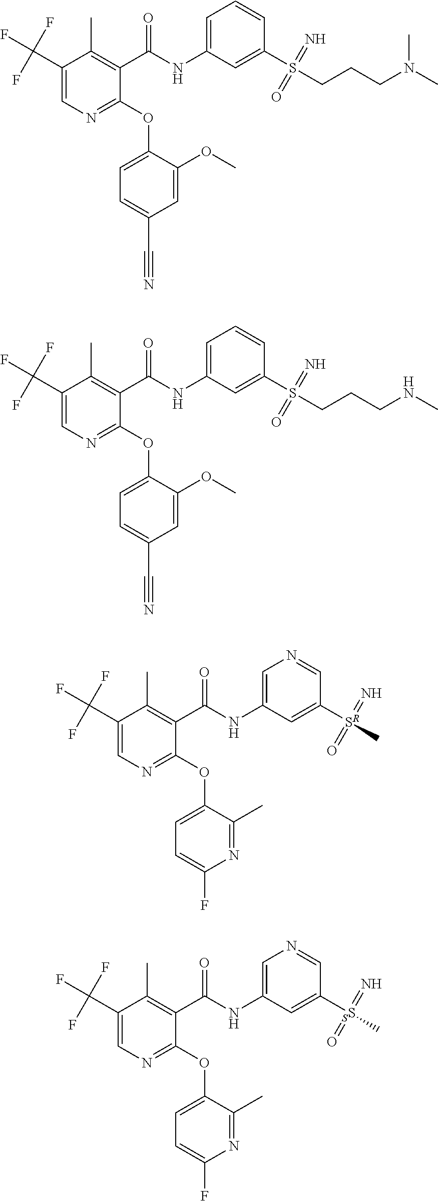

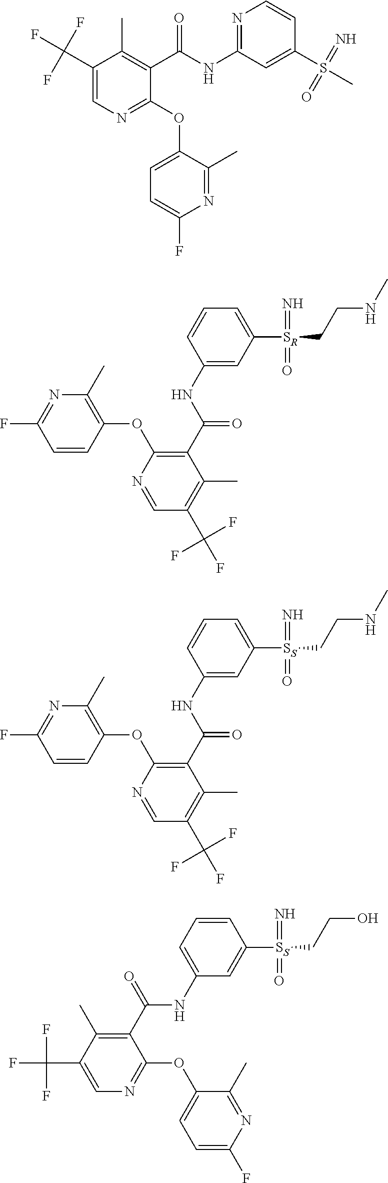

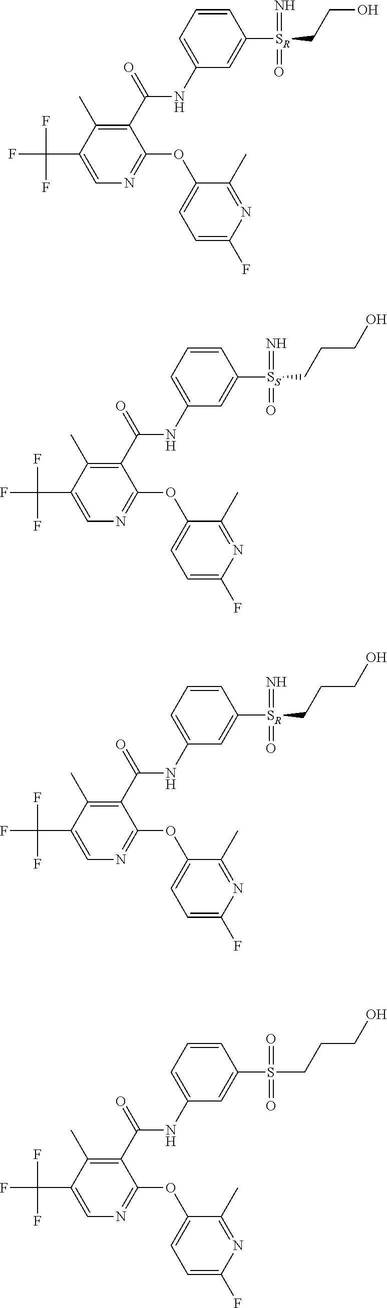

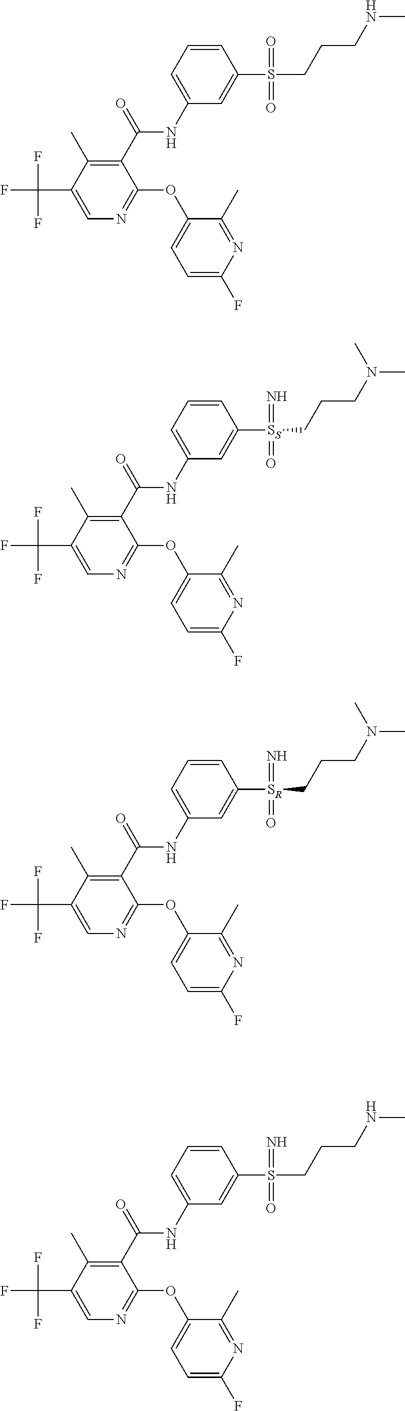

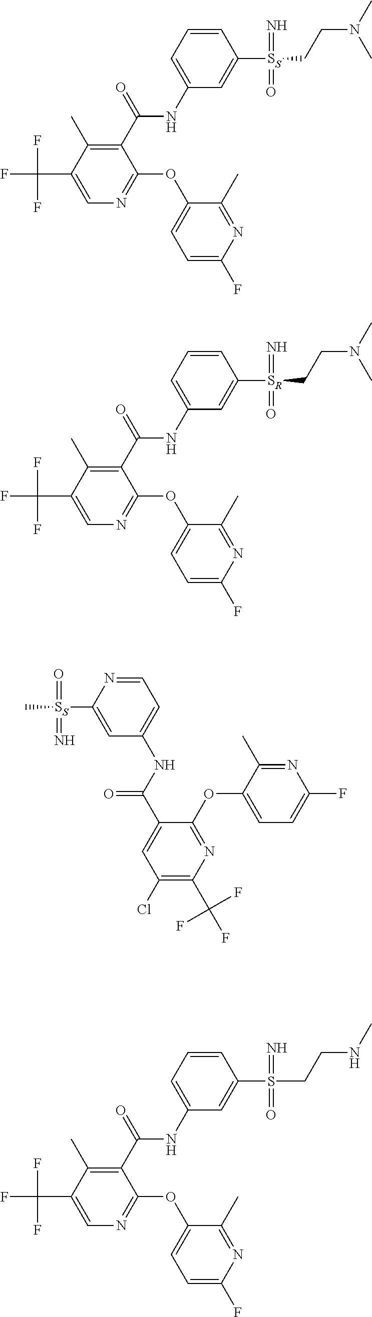

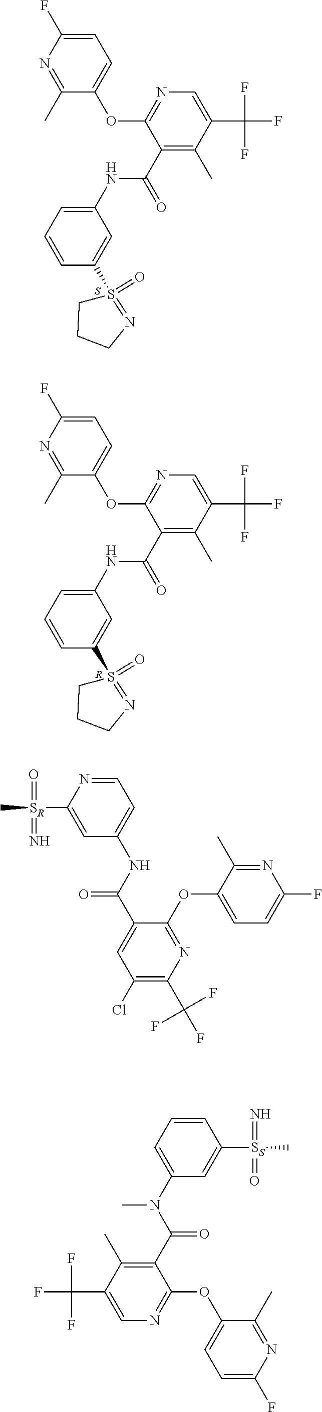

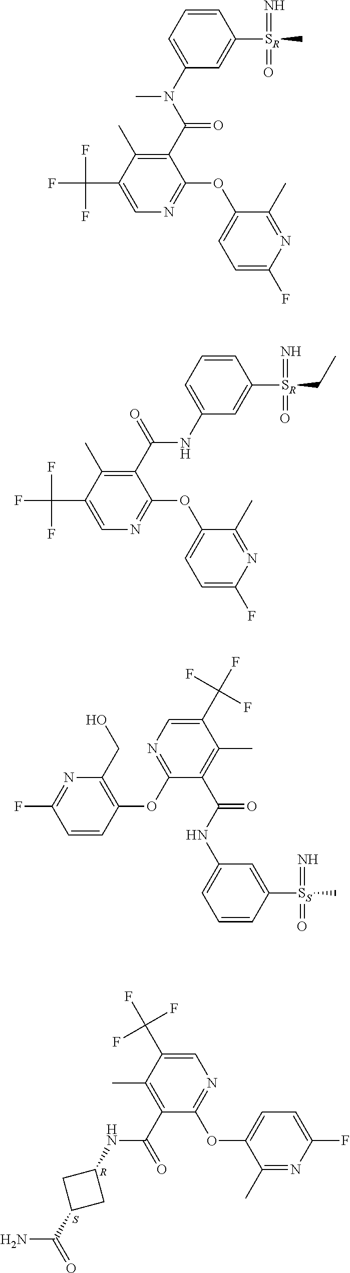

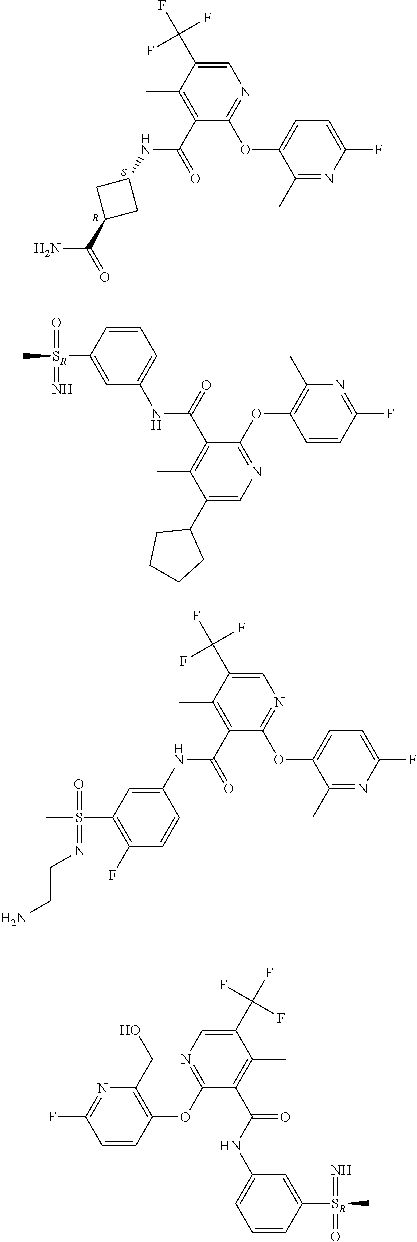

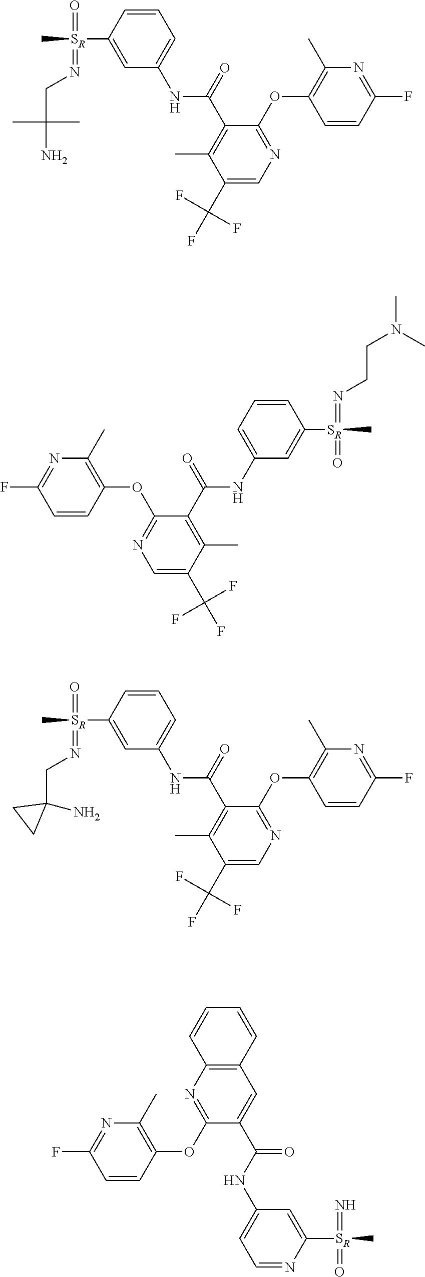

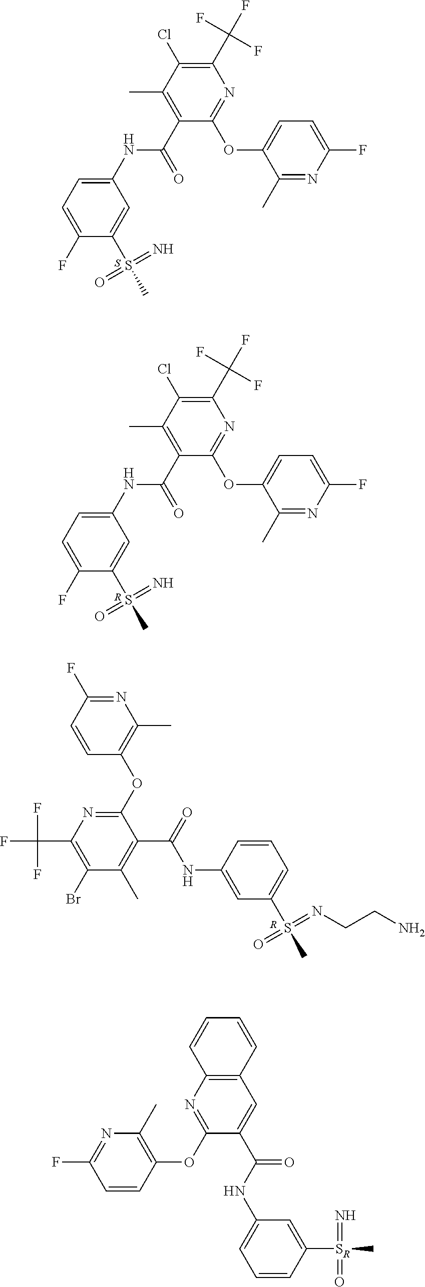

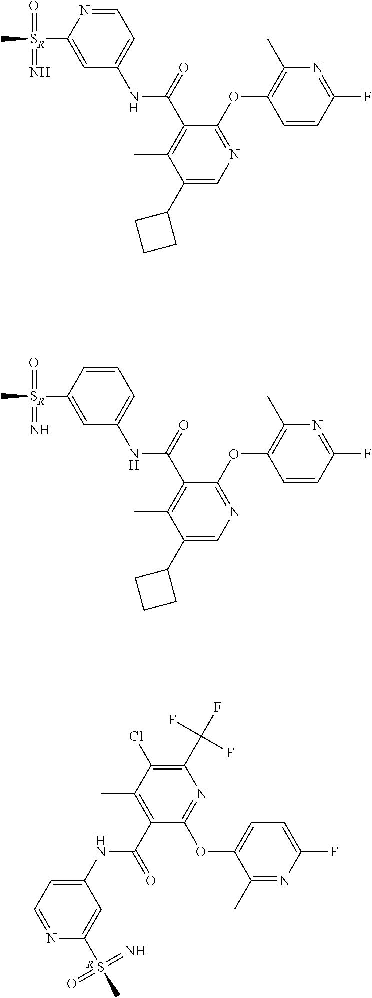

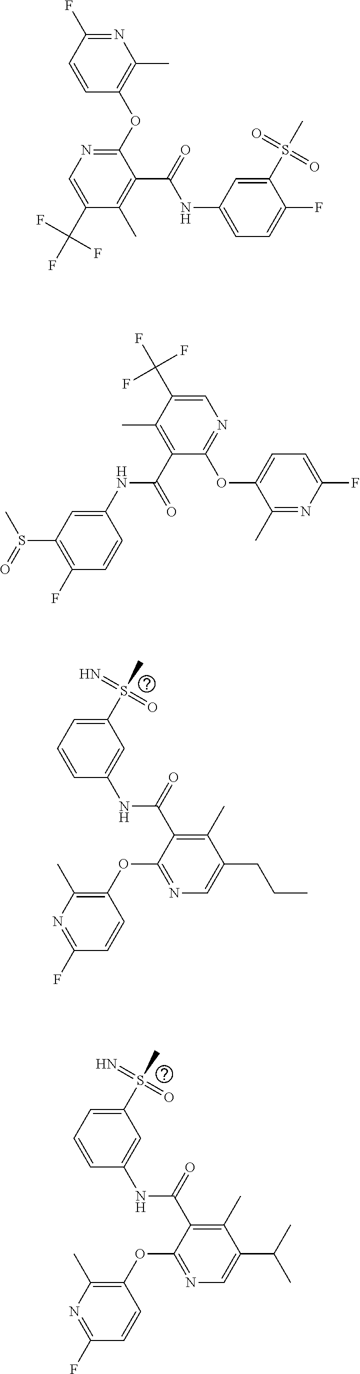 [SMILES: Cc1nc(F)ccc1Oc1ncc(C(F)(F)F)c(C)c1C(=O)Nc1ccc(F)c(S(C)(=O)=O)c1 ; Cc1nc(F)ccc1Oc1ncc(C(F)(F)F)c(C)c1C(=O)Nc1ccc(F)c(S(C)=O)c1 ; CCCc1cnc(Oc2ccc(F)nc2C)c(C(=O)Nc2cccc([S@](C)(=N)=O)c2)c1C ; Cc1nc(F)ccc1Oc1ncc(C(C)C)c(C)c1C(=O)Nc1cccc([S@](C)(=N)=O)c1]

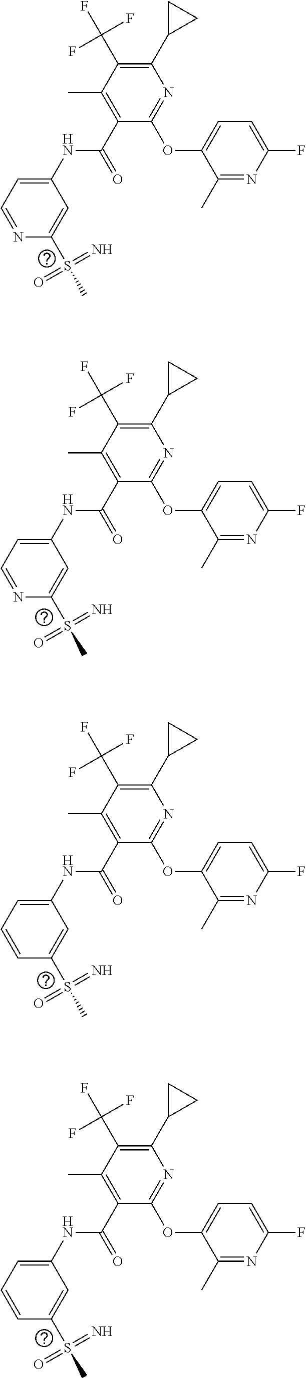 [SMILES: Cc1nc(F)ccc1Oc1nc(C2CC2)c(C(F)(F)F)c(C)c1C(=O)Nc1ccnc([S@@](C)(=N)=O)c1 ; Cc1nc(F)ccc1Oc1nc(C2CC2)c(C(F)(F)F)c(C)c1C(=O)Nc1ccnc([S@](C)(=N)=O)c1 ; Cc1nc(F)ccc1Oc1nc(C2CC2)c(C(F)(F)F)c(C)c1C(=O)Nc1cccc([S@@](C)(=N)=O)c1 ; Cc1nc(F)ccc1Oc1nc(C2CC2)c(C(F)(F)F)c(C)c1C(=O)Nc1cccc([S@](C)(=N)=O)c1]

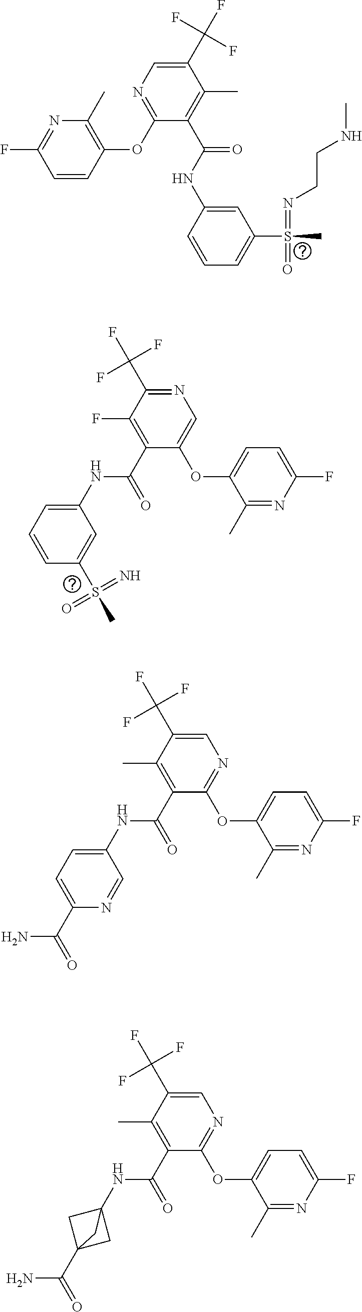 [SMILES: CNCCN=[S@](C)(=O)c1cccc(NC(=O)c2c(Oc3ccc(F)nc3C)ncc(C(F)(F)F)c2C)c1 ; Cc1nc(F)ccc1Oc1cnc(C(F)(F)F)c(F)c1C(=O)Nc1cccc([S@](C)(=N)=O)c1 ; Cc1nc(F)ccc1Oc1ncc(C(F)(F)F)c(C)c1C(=O)Nc1ccc(C(N)=O)nc1 ; Cc1nc(F)ccc1Oc1ncc(C(F)(F)F)c(C)c1C(=O)NC12CC(C(N)=O)(C1)C2]

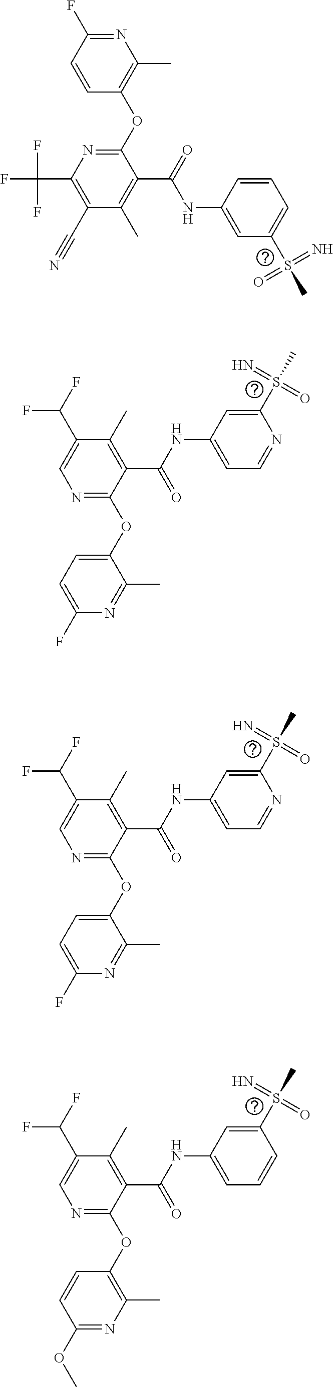 [SMILES: Cc1nc(F)ccc1Oc1nc(C(F)(F)F)c(C#N)c(C)c1C(=O)Nc1cccc([S@](C)(=N)=O)c1 ; Cc1nc(F)ccc1Oc1ncc(C(F)F)c(C)c1C(=O)Nc1ccnc([S@@](C)(=N)=O)c1 ; Cc1nc(F)ccc1Oc1ncc(C(F)F)c(C)c1C(=O)Nc1ccnc([S@](C)(=N)=O)c1 ; COc1ccc(Oc2ncc(C(F)F)c(C)c2C(=O)Nc2cccc([S@](C)(=N)=O)c2)c(C)n1]

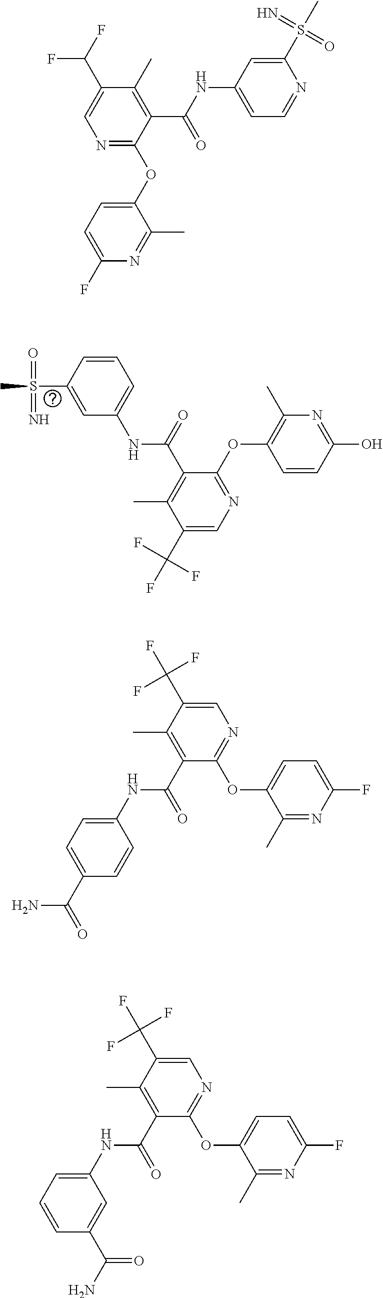 [SMILES: Cc1nc(F)ccc1Oc1ncc(C(F)F)c(C)c1C(=O)Nc1ccnc(S(C)(=N)=O)c1 ; Cc1nc(O)ccc1Oc1ncc(C(F)(F)F)c(C)c1C(=O)Nc1cccc([S@](C)(=N)=O)c1 ; Cc1nc(F)ccc1Oc1ncc(C(F)(F)F)c(C)c1C(=O)Nc1ccc(C(N)=O)cc1 ; Cc1nc(F)ccc1Oc1ncc(C(F)(F)F)c(C)c1C(=O)Nc1cccc(C(N)=O)c1]

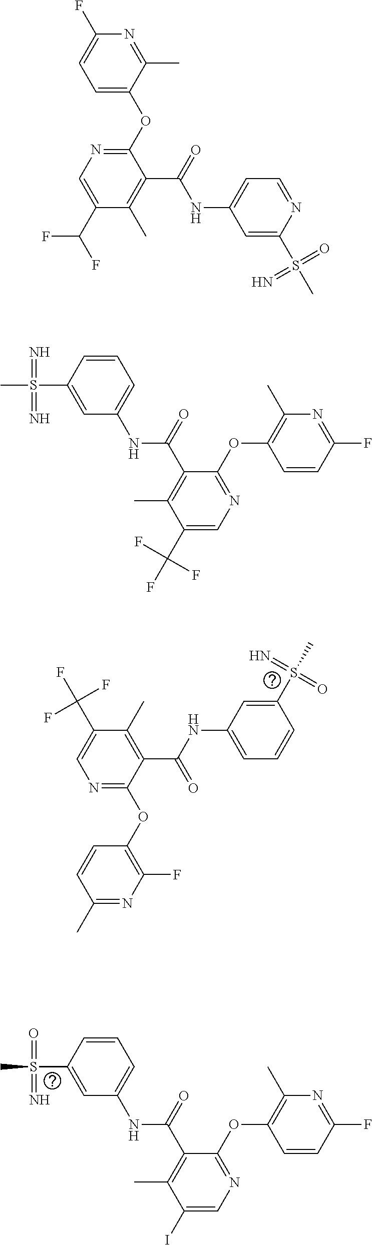 [SMILES: Cc1nc(F)ccc1Oc1ncc(C(F)F)c(C)c1C(=O)Nc1ccnc(S(C)(=N)=O)c1 ; Cc1nc(F)ccc1Oc1ncc(C(F)(F)F)c(C)c1C(=O)Nc1cccc(S(C)(=N)=N)c1 ; Cc1ccc(Oc2ncc(C(F)(F)F)c(C)c2C(=O)Nc2cccc([S@@](C)(=N)=O)c2)c(F)n1 ; Cc1nc(F)ccc1Oc1ncc(I)c(C)c1C(=O)Nc1cccc([S@](C)(=N)=O)c1]

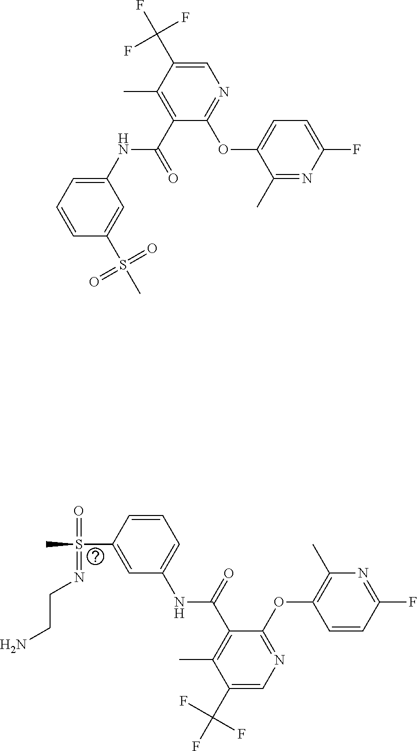 [SMILES: Cc1nc(F)ccc1Oc1ncc(C(F)(F)F)c(C)c1C(=O)Nc1cccc(S(C)(=O)=O)c1 ; Cc1nc(F)ccc1Oc1ncc(C(F)(F)F)c(C)c1C(=O)Nc1cccc([S@@](C)(=O)=NCCN)c1]

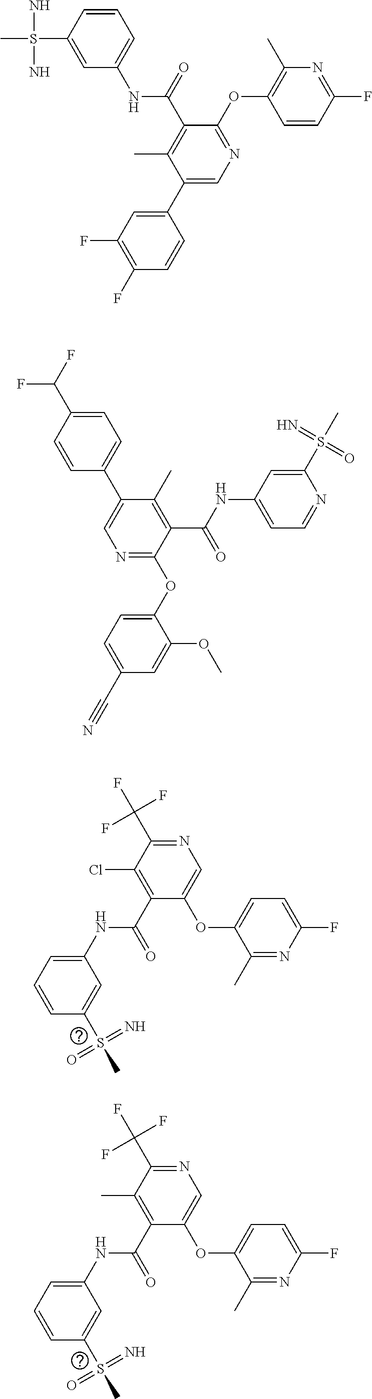

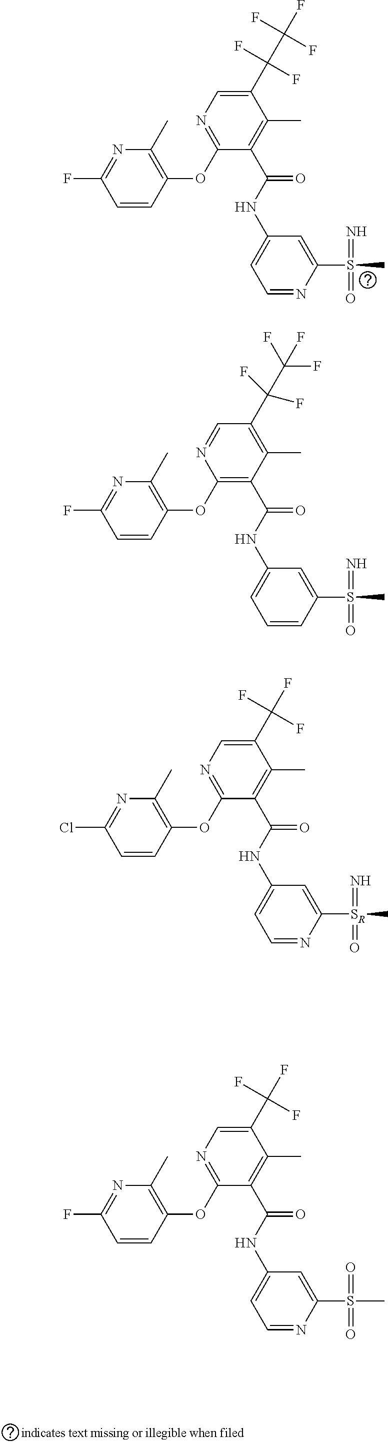

The third set of compounds, provided herein, are prepared by methods and procedures described below. The intermediates described in this section may be relied upon for preparation of third set of compounds.

Methods of making the compounds of the present invention, and intermediates used in their synthesis, are provided in the General Synthetic Schemes and Specific Syntheses Procedures below. Chemicals were purchased from standard commercial vendors and used as received unless otherwise noted. Otherwise, their preparation is facile and known to one of ordinary skill in the art, or it is referenced or described herein. Abbreviations are consistent with those in the ACS Style Guide. “dry” glassware means oven/desiccator dried. Solvents were ACS grade unless otherwise noted.

All reactions were performed in flame-dried or oven-dried glassware under a positive pressure of dry nitrogen or dry argon and were stirred magnetically unless otherwise indicated. Chemicals were purchased from standard commercial vendors and used as received unless otherwise noted. Yields are not optimized. The chemical names were generated using the ChemDraw Professional 19.1, available from PerkinElmer or chemAxon.

Reactions were monitored by thin layer chromatography (TLC) using 0.25 mm silica gel 60 F254 plates purchased from EMD MILLIPORE™. Purification was performed with Biotage Isolera One Flash Chromatography Instrument or purified using one of the preparative HPLC methods mentioned below.

### Prep Method 1

Equipment: Shimadzu LCMS 2020 mass-directed preparative HPLC System; column: Gemini 5 um C₁₈ column, 150*21.2 mm; General gradient: 30% to 90% MeCN/H₂O containing 0.1% HCOOH, gradient may be slight adjusted for specific compound; Flow rate: 20 mL/min; Column temperature: ambient temperature; UV Wavelength: 214 and 254 nm;

### Prep Method 2

Equipment: Shimadzu LC-20AP Preparative HPLC System; column: Gemini 5 um C₁₈ column, 150*21.2 mm; General gradient: 30% to 90% MeCN/120 containing 0.1% TFA, gradient may be slight adjusted for specific compound; Flow rate: 20 mL/min; Column temperature: ambient temperature; UV Wavelength: 214 and 254 nm.

### Prep Method 3

Equipment: Shimadzu LC-20AP Preparative HPLC System; column: Gemini 5 um C₁₈ column, 150×21.2 mm; General gradient: 30% to 90% MeCN/H₂O containing 0.05% ammonia, gradient may be slight adjusted for specific compound; Flow rate: 20 mL/min; Column temperature: ambient temperature; UV Wavelength: 214 and 254 nm.

Analytical LCMC were collected using one of following methods.

### Analytical Method 1

Equipment: Shimadzu LCMS 2020 Mass Spectrometer; Column: HALO C₁₈ 2.7 μm, 3.0 mm×30 mm; Mobile Phase: MeCN (0.05% HCOOH)—Water (0.05% HCOOH); Gradient: MeCN from 5% to 95% over 1.4 min, hold 0.6 min, total run time is 2.5 min; Flow rate: 1.8 mL/min; Column temperature: 50° C.; Wavelength: 214 and 254 nm PDA.

### Analytical Method 2

Equipment: Shimadzu LCMS 2020 Mass Spectrometer; XBridge BEH C₁₈ 2.5 μm, 3.0 mm×30 mm Mobile Phase: MeCN—Water (0.1% NH₄OH); Gradient: MeCN from 5% to 95% over 1.8 min, hold 0.7 min, total run time is 3.0 min; Flow rate: 1.0 mL/min; Column temperature: 50° C.; Wavelength: 214 and 254 nm PDA.

Analytical Method 3 Equipment: Shimadzu LCMS 2020 Mass Spectrometer; Column: HALO C₁₈ 2.7 μm, 3.0 mm×30 mm Mobile Phase: MeCN (0.05% TFA)—Water (0.05% TFA); Gradient: MeCN from 5% to 95% over 1.4 min, hold 0.6 min, total run time is 2.5 min; Flow rate: 1.8 mL/min; Column temperature: 50° C.; Wavelength: 214 and 254 nm PDA.

SFC chiral resolution was performed on Shimadzu Nexera UC Preparative SFC System (SFE-30A, LC-30AD_{SF}, SFC-30A) using following methods:

### Analytical Method 4

Column: Daicel chiralpak-AS-H 5 μm 250×20 mm; Mobile Phase: CO₂/MeOH [0.1% NH₃ (7M in MeOH)], CO₂/MeOH ratio varies for different compounds; Oven temperature: 40° C.; Flow rate: 38 mL/min.

### Analytical Method 5

Column: Daicel chiralpak-OJ-H 5 μm 250*20 mm; Mobile Phase: CO₂/MeOH (0.1% HCOOH), CO₂/MeOH ratio varies for different compounds; Oven temperature: 40° C.; Flow rate: 38 mL/min.

### Analytical Method 6

Column: Daicel chiralpak-OD-H 5 μm 250×20 mm; Mobile Phase: CO₂/MeOH, ratio varies for different compounds; Oven temperature: 40° C.; Flow rate: 38 mL/min.

### Analytical Method 7

Column: Daicel chiralpak-AD-H 5 μm 250×20 mm; Mobile Phase: CO₂/i-PrOH, ratio varies for different compounds; Oven temperature: 40° C.; Flow rate: 38 mL/min.

### Analytical Method 8

Column: Daicel chiralpak-IC 5 μm 250×20 mm; Mobile Phase: CO₂/EtOH, ratio varies for different compounds; Oven temperature: 40° C.; Flow rate: 38 mL/min.

Unless otherwise stated, ¹H nuclear magnetic resonance spectroscopy (NMR) spectra were recorded on a Bruker AVANCE NEO 400 MHz Digital NMR Spectrometer. Chemical shifts, 6, are quoted in parts per million (ppm) relative to TMS and calibrated using residual un-deuterated solvent as an internal reference. The following abbreviations are used to denote the multiplicities and general assignments: s (singlet), d (doublet), t (triplet), q (quartet), dd (doublet of doublets), ddd (doublet of doublet of doublets), dt (doublet of triplets), dq (doublet of quartets), hep (heptet), m (multiplet), pent (pentet), td (triplet of doublets), qd (quartet of doublets), app. (apparent) and br. (broad). Coupling constants, J, are quoted to the nearest 0.1 Hz.

### General Synthetic Schemes

Several methods for preparing the compounds of this invention are illustrated in the following Schemes and Examples. The present invention further provides processes for the preparation of compounds of structural Formula I as defined above. In some cases, the order of carrying out the foregoing reaction schemes may be varied to facilitate the reaction or to avoid unwanted reaction products. The following examples are provided for the purpose of illustration only and are not to be construed as limitations on the disclosed invention.

Compounds of Formula (I) may be prepared from A-1 starting materials or intermediates but not limited to methyl 4,6-dichloropyridazine-3-carboxylate, 2,6-dichloro-3-(trifluoromethyl)pyridine, 2,4-dichloro-5-nitropyridine, methyl 2-hydroxy-4-methylnicotinate, 2-bromo-3,5-dichloroisonicotinic acid, methyl 2,4-dichloro-6-methylpyrimidine-5-carboxylate, methyl 5-bromo-2-chloro-4-methylnicotinate, ethyl 5-bromo-3-fluoro-2-iodoisonicotinate, 2,6-dichloro-4-methylnicotinic acid, ethyl 2-chloro-4-methyl-6-(trifluoromethyl)nicotinate, methyl 2-chloro-5-nitronicotinate, 2-chloro-4,6-dimethylnicotinic acid, that are commercially available, synthesized using published procedures, or synthesized by methods known to those skilled in the art. Modifications may be made to the starting materials and intermediates to prepare still other intermediates that possess alternate or additional groups such as alkyl, fluoroalkyl, trifluoromethyl, cyano, halogen, chloro, bromo, iodo, amino, nitro, aryl, heteroaryl, carboalkoxy, alkoxy, aryloxy, heteroaryloxy, and the like. Methodology used to convert above starting materials into Compounds of Formula (I) are shown for each of the Examples.

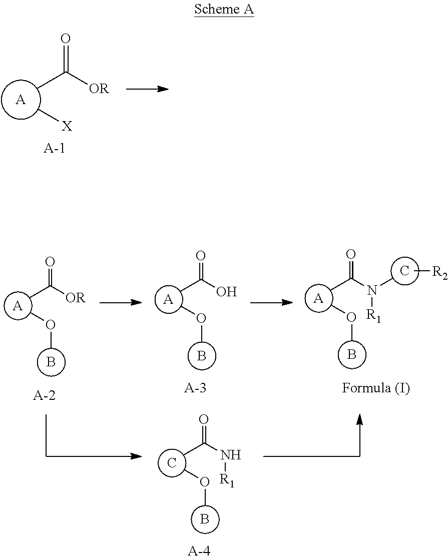

As illustrated in Scheme A, in general, compounds of Formula (I) may be synthesized from an A-1 starting material or intermediate. Ring A and X of A-1 may first be modified to give an intermediate A-1 suitable for subsequent modification. Modifications can include, but are not limited to, exchange of a halogen for trifluoromethyl, metal-catalyzed coupling to introduce an alkyl, aromatic, or heteroaromatic group, treatment with nitric acid to introduce a nitro group, reduction of a nitro group to an amino group, or other transformations known to those skilled in the art. The X group of A-1 may be displaced by various substituted phenols or heteroaromatic alcohols in the presence of base, such as K₂CO₃, Cs₂CO₃, NaH, KH or other organic bases to provide intermediates of type A-2. Intermediates of type A-2 may or may not be further modified on Ring A and the carboxylate ester is subsequently hydrolyzed with KOH or LiOH in an aqueous solvent, to give the carboxylic acid intermediate A-3. Intermediates A-3 may be reacted with a substituted aniline or heteroaryl aniline using standard amide coupling reagents, not limited to HATU, TBTU, EDC or T3P in organic solvents and base, such as DIEA, to give Compounds of Formula (I). If desired, Compounds of Formula (I) may be further modified to provide additional Compounds of Formula (I). Alternatively, Intermediate A-2 may be treated with ammonia, a primary amine (R₁NH₂), or a dialkylaluminum amide to give carboxamide A-4 which can undergo metal-catalyzed coupling with a halo-substituted aniline or heteroaryl aniline to give Compounds of Formula (I).

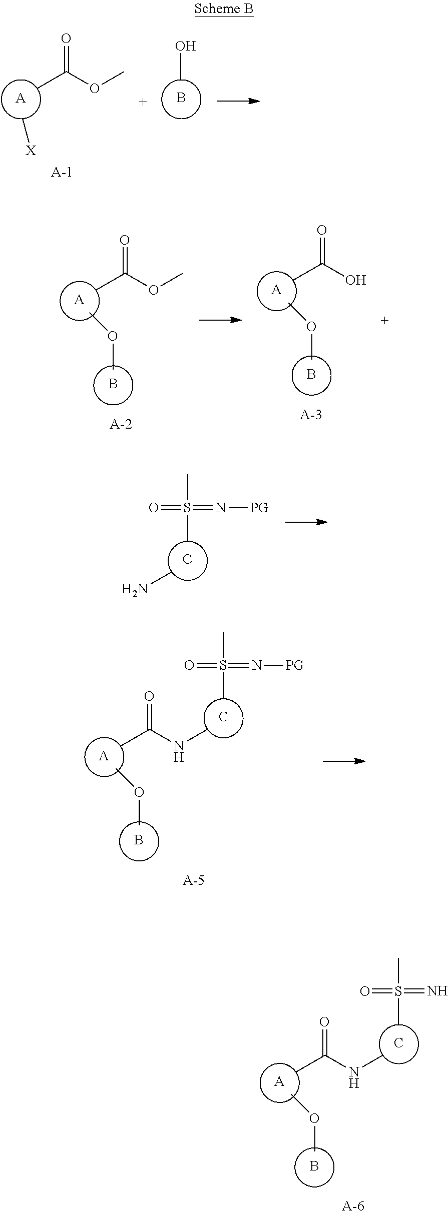

As illustrated in Scheme B, in general, compounds of the invention can be prepared by reacting substituted compound A-1 with an aryl- or heteroaryl-alcohol B using a base such as DIEA or inorganic base such as K₂CO₃ or Cs₂CO₃ to afford intermediate A-2. Intermediate A-2 can be converted to corresponding acid A-3 by treating A-2 with base such as KOH or LiOH or NaOH in aqueous EtOH or MeOH or a mixture of MeOH/THF/H₂O. Intermediates A-5 can be formed either by treating A-3 and amine C, utilizing amide coupling conditions or by activation of appropriately functionalized carboxylic acid A-4 with (COCl)₂ or POCl₃ and with amine C base such as DIEA or pyridine in DCM, DMF or THF. The compounds of formula A-6 can be formed by removing a protecting group, such as Boc under acidic conditions. In some instances, A-6 can be separated to the corresponding R and S isomers using chiral HPLC. R- and S-isomers also can be prepared by coupling of the acid to enantiomerically pure amine C followed deprotection.

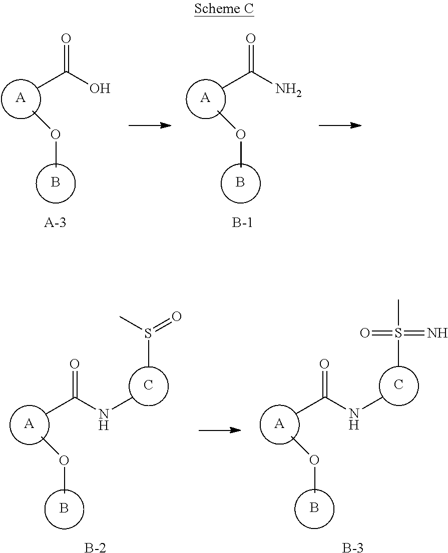

As illustrated in Scheme C, in general, compounds of the invention can be prepared by activation of appropriately functionalized carboxylic acid A-3 in organic solvent with either (COCl)₂ or SOCl₂ followed by addition of NH₄OH to afford B-1. Intermediate B—I can then be brought together with materials of variously substituted Br compounds, utilizing Xantphos-Pd-G2 mediated coupling conditions to deliver intermediate B-2. The compounds of formula B-2 treated with ammonium carbonate or ammonium carbamate and (diacetoxyiodo)benzene (PIDA) in methanol to deliver compound of formula B-3. In some instances, B-3 was separated corresponding R and S isomers using chiral HPLC conditions.

### Intermediate 1

### 2-((6-Fluoro-2-methylpyridin-3-yl)oxy)-4-methyl-5-(trifluoromethyl)nicotinic acid

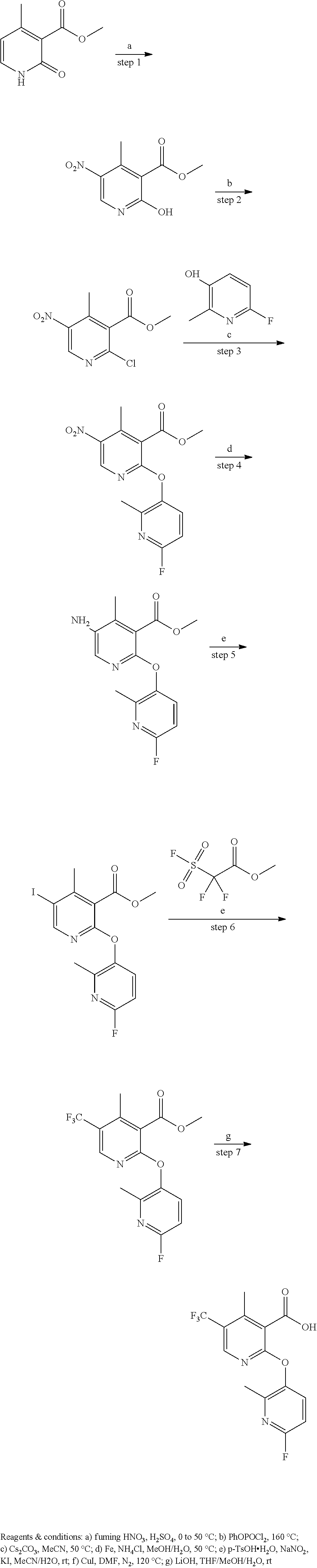

Step 1. methyl 2-hydroxy-4-methyl-5-nitronicotinate: To a stirred solution of methyl 2-hydroxy-4-methylnicotinate (40 g, 0.24 mol) in H₂SO₄ (200 mL) was added fuming HNO₃ (13 mL) dropwise at 0° C. The mixture was heated to 50° C. for 5 hours. The resulting mixture was poured into ice water (3 L). The precipitate was collected by filtration, washed with water, and dried under vacuum to give crude methyl 2-hydroxy-4-methyl-5-nitronicotinate (26 g, 51% yield) as a yellow solid. LCMS (ESI) calcd. for C₈H₉N₂O₅ [M+H]⁺ m/z 213.05, found 212.90.

Step 2. methyl 2-chloro-4-methyl-5-nitronicotinate: A solution of methyl 2-hydroxy-4-methyl-5-nitronicotinate 5 (8.0 g, 37.8 mmol) in phenyl dichlorophosphate (40 mL) was heated to 160° C. for 2 hours. The resulting solution was cooled to room temperature, quenched with water (100 mL) and extracted with EtOAc (50 mL×3). The combined organic layers were washed with brine, dried over sodium sulfate, concentrated under vacuum. The residue was purified by flash column chromatography on silica gel (PE/EtOAc=10/1) to provide methyl 2-chloro-4-methyl-5-nitronicotinate (6 g, 69% yield) as a light-yellow solid. LCMS (ESI) calcd. for C₈H₈ClN₂O₄ [M+H]⁺ m/z 231.02, found 231.00.

Step 3. methyl 2-((6-fluoro-2-methylpyridin-3-yl)oxy)-4-methyl-5-nitronicotinate: To a solution of methyl 2-chloro-4-methyl-5-nitronicotinate (4 g, 17 mmol) and 6-fluoro-2-methylpyridin-3-ol (2.31 g, 18 mmol) in MeCN (30 mL) was added Cs₂CO₃ (8.46 g, 26 mmol). The mixture was heated at 50° C. for 1 hours. After the reaction was completed, the mixture concentrated under vacuum and the residue was directly purified by flash column chromatography on silica gel (PE/EtOAc=4/1 to 3/1) to provide methyl 2-((6-fluoro-2-methylpyridin-3-yl)oxy)-4-methyl-5-nitronicotinate (4.2 g, 77% yield) as a light-yellow solid. LCMS (ESI) calcd. for C₁₄H₁₃FN₃O₅ [M+H]⁺ m/z 322.09, found 322.00.

Step 4. methyl 5-amino-2-((6-fluoro-2-methylpyridin-3-yl)oxy)-4-methylnicotinate: A solution of methyl 2-((6-fluoro-2-methylpyridin-3-yl)oxy)-4-methyl-5-nitronicotinate 9 (4.2 g, 13.1 mmol) in MeOH (30 mL) and water (10 mL) was added NH₄Cl (4.91 g, 91.7 mmol), Fe (3.66 g, 65.5 mmol). The mixture was heated at 60° C. for 2 hours. After the reaction was completed. The mixture was filtered through celite. The filtrate was diluted with water (100 mL) and extracted with EtOAc (100 mL×3). The combined organic layers were washed with brine, dried over sodium sulfate, concentrated under vacuum to provide methyl 5-amino-2-((6-fluoro-2-methylpyridin-3-yl)oxy)-4-methylnicotinate (3.7 g, 97% yield) as a yellow solid. LCMS (ESI) calcd. for C₁₄H₁₅FN₃O₃ [M+H]⁺ m/z 292.11, found 291.95.

Step 5. methyl 2-((6-fluoro-2-methylpyridin-3-yl)oxy)-5-iodo-4-methylnicotinate: To a solution of TsOH-H₂O (3.94 g, 20.7 mmol) and methyl 5-amino-2-((6-fluoro-2-methylpyridin-3-yl)oxy)-4-methylnicotinate (2 g, 6.9 mmol) in MeCN (60 mL) was added a solution of NaNO₂ (950 mg, 13.8 mmol) and KI (2.86 g, 17.3 mmol) in H₂O (10 mL) at 0° C. The mixture was stirred at room temperature for 2.5 h. After the reaction was completed, the mixture was quenched with water (100 mL), adjusted to pH=8-9 with saturated aqueous NaHCO₃. The solution was extracted with EtOAc (200 mL×3). The combined organic layers were washed with brine, dried over sodium sulfate, concentrated under vacuum. The residue was purified by flash column chromatography on silica gel (PE/EtOAc=10/1 to 1/1) to provide methyl 2-((6-fluoro-2-methylpyridin-3-yl)oxy)-5-iodo-4-methylnicotinate (2.2 g, 78% yield) as yellow solid. LCMS (ESI) calcd. for C₁₄H₁₃FIN₂O₃ [M+H]⁺ m/z 403.00, found 402.90.

Step 6. methyl 2-((6-fluoro-2-methylpyridin-3-yl)oxy)-4-methyl-5-(trifluoromethyl)nicotinate: To a solution of methyl 2-((6-fluoro-2-methylpyridin-3-yl)oxy)-5-iodo-4-methylnicotinate (1.2 g, 3 mmol) and CuI (1.14 g, 6 mmol) in DMF (30 mL) was added methyl 2,2-difluoro-2-(fluorosulfonyl) acetate (2.88 g, 15 mmol) dropwise at room temperature under an atmosphere of N2. The mixture was heated at 120° C. for 6 hours. After the reaction was completed, the resulting solution was diluted with water (100 mL) and extracted with EtOAc (100 mL×3). The combined organic phases were washed with brine, dried over sodium sulfate, concentrated under vacuum. The residue was purified by flash column chromatography on silica gel (PE/EtOAc=10/1 to 1/1) to give methyl 2-((6-fluoro-2-methylpyridin-3-yl)oxy)-4-methyl-5-(trifluoromethyl)nicotinate (1 g, 93.3% yield). LCMS (ESI) calcd. for C₁₅H₁₃F₄N₂O₃ [M+H]f m/z 345.09, found 344.95.

Step 7. 2-((6-fluoro-2-methylpyridin-3-yl)oxy)-4-methyl-5-(trifluoromethyl)nicotinic acid: To a solution of methyl 2-((6-fluoro-2-methylpyridin-3-yl)oxy)-4-methyl-5-(trifluoromethyl)nicotinate 12 (1 g, 2.9 mmol) in THF/MeOH/H₂O (1/1/1, 30 mL) was added LiOH·H₂O (730 mg, 17.4 mmol) at room temperature. The reaction mixture was stirred at room temperature for 18 hours. After the reaction was completed, the mixture was concentrated. The residue was adjusted to pH=3-4 with aqueous HCl (1M). Then the solution was extracted with EtOAc (80 mL×3). The combined organic layers were washed with brine, dried over sodium sulfate, concentrated under vacuum to provide 2-((6-fluoro-2-methylpyridin-3-yl)oxy)-4-methyl-5-(trifluoromethyl)nicotinic acid (900 mg, 94% yield) as a white solid. LCMS (ESI) calcd. for C₁₄H₁₁F₄N₂O₃ [M+H]⁺ m/z 331.07, found 330.90.

### Intermediate 2

### 5-bromo-2-((6-fluoro-2-methylpyridin-3-yl)oxy)-4-methylnicotinic acid

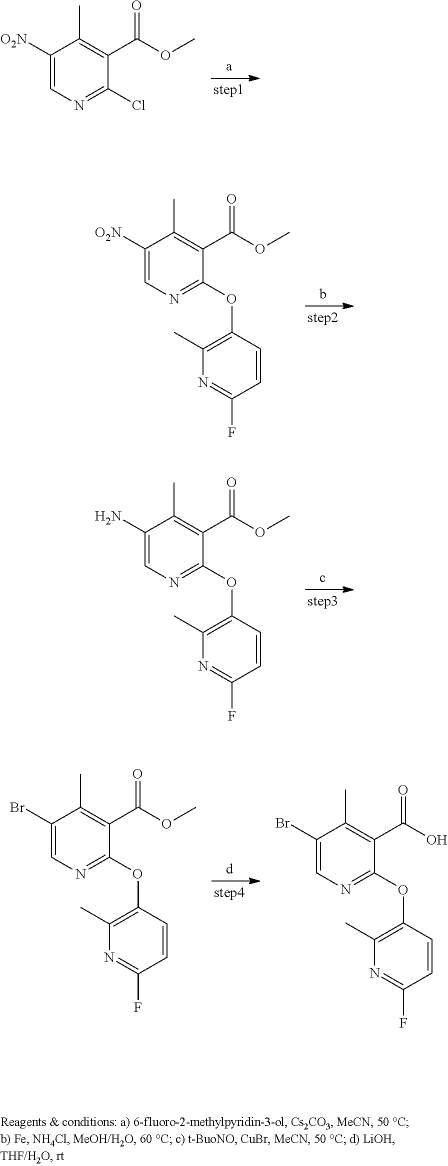 [SMILES: COC(=O)c1c(Cl)ncc([N+](=O)[O-])c1C ; COC(=O)c1c(Oc2ccc(F)nc2C)ncc([N+](=O)[O-])c1C ; COC(=O)c1c(Oc2ccc(F)nc2C)ncc(N)c1C ; COC(=O)c1c(Oc2ccc(F)nc2C)ncc(Br)c1C ; Cc1nc(F)ccc1Oc1ncc(Br)c(C)c1C(=O)O]

Step 1: methyl 2-((6-fluoro-2-methylpyridin-3-yl)oxy)-4-methyl-5-nitronicotinate: To a solution of methyl 2-chloro-4-methyl-5-nitronicotinate (3.6 g, 15.7 mmol) and 6-fluoro-2-methylpyridin-3-ol (2.0 g, 15.7 mmol) in MeCN (60 mL) was added Cs₂CO₃ (10.0 g, 31.5 mmol). The mixture was heated at 50° C. for 1 hour. After the reaction was completed, the mixture concentrated under vacuum and directly purified by flash column chromatography on silica gel (PE/EtOAc=3/1) to provide methyl 2-((6-fluoro-2-methylpyridin-3-yl)oxy)-4-methyl-5-nitronicotinate (2.7 g, 53.6%) as a light-yellow solid. LCMS (ESI) calcd. for C₁₄H₁₃FN₃O₅ [M+H]⁺ m/z 322.09, found 321.95.

Step 2: methyl 5-amino-2-((6-fluoro-2-methylpyridin-3-yl)oxy)-4-methylnicotinate: A solution of methyl 2-((6-fluoro-2-methylpyridin-3-yl)oxy)-4-methyl-5-nitronicotinate (2.5 g, 7.8 mmol) in MeOH (30 mL) and water (10 mL) was added NH₄Cl (2.71 g, 51.1 mmol), Fe (2.04 g, 36.5 mmol). The mixture was heated to 60° C. for 1 hour. After the reaction was completed, the mixture was filtered through celite. The filtrate was diluted with water (100 mL) and extracted with EtOAc (50 mL×3). The combined organic phases were washed with brine, dried over sodium sulfate, concentrated under vacuum to provide methyl 5-amino-2-((6-fluoro-2-methylpyridin-3-yl)oxy)-4-methylnicotinate (1.4 g, 61.9%) as a yellow solid. LCMS (ESI) calcd. for C₁₄H₁₅FN₃O₃ [M+H]⁺ m/z 292.11, found 292.00.

Step 3: methyl 5-bromo-2-((6-fluoro-2-methylpyridin-3-yl)oxy)-4-methylnicotinate: To a solution of methyl 5-amino-2-((6-fluoro-2-methylpyridin-3-yl)oxy)-4-methylnicotinate (1.3 g, 4.46 mmol) and CuBr (638 mg, 4.46 mmol) in MeCN (30 mL) was added t-BuONO (459 mg, 4.46 mmol) dropwise at room temperature. The reaction solution was heated at 50° C. for 1 hour. After the reaction was completed, the resulting solution was diluted with water (80 mL) and extracted with EtOAc (40 mL×3). The combined organic phases were washed with brine, dried over sodium sulfate, concentrated under vacuum. The residue was purified by flash column chromatography on silica gel (PE/EtOAc=10/1 to 5/1) to give methyl 5-bromo-2-((6-fluoro-2-methylpyridin-3-yl)oxy)-4-methylnicotinate (620 mg, 39%) as a yellow solid. LCMS (ESI) calcd. for C₁₄H₁₃BrFN₂O₃ [M+H]⁺ m/z 357.01, found 356.85.

Step 4: 5-bromo-2-((6-fluoro-2-methylpyridin-3-yl)oxy)-4-methylnicotinic acid: A solution of methyl 5-bromo-2-((6-fluoro-2-methylpyridin-3-yl)oxy)-4-methylnicotinate (620 mg, 1.75 mmol) in THE (5 mL), MeOH (5 mL) and H₂O (5 mL) was added LiOH·H₂O (483 mg, 10.5 mmol) at room temperature. The reaction mixture was stirred at room temperature for 18 hours. After the reaction was completed, the mixture was concentrated. The residue was adjusted to pH=3-4 with 1N HCl. Then the aqueous solution was extracted with EtOAc (40 mL×3). The combined organic phases were washed with brine, dried over sodium sulfate, concentrated under vacuum to provide 5-bromo-2-((6-fluoro-2-methylpyridin-3-yl)oxy)-4-methylnicotinic acid (490 mg, 82%) as a brown solid. LCMS (ESI) calcd. for C₁₃H₁₁BrFN₂O₃ [M+H]⁺ m/z 341.00, found 340.85.

### Intermediate 3

### 3-((6-fluoro-2-methylpyridin-3-yl)oxy)-5-methyl-6-(trifluoromethyl)pyridazine-4-carboxylic acid

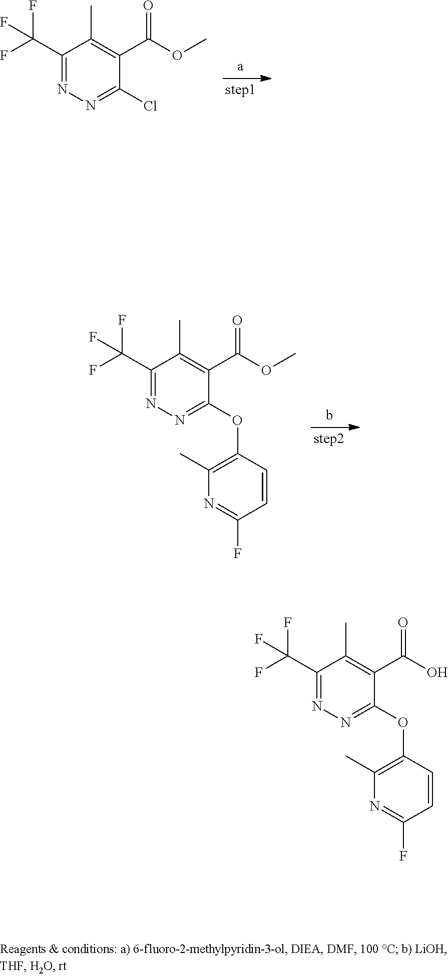 [SMILES: COC(=O)c1c(Cl)nnc(C(F)(F)F)c1C ; COC(=O)c1c(Oc2ccc(F)nc2C)nnc(C(F)(F)F)c1C ; Cc1nc(F)ccc1Oc1nnc(C(F)(F)F)c(C)c1C(=O)O]

Step 1: methyl 3-((6-fluoro-2-methylpyridin-3-yl)oxy)-5-methyl-6-(trifluoromethyl)pyridazine-4-carboxylate: A mixture of methyl 3-chloro-5-methyl-6-(trifluoromethyl)pyridazine-4-carboxylate (500 mg, 1.97 mmol), 6-fluoro-2-methylpyridin-3-ol (375 mg, 2.95 mmol), DIEA (381 mg, 2.95 mmol), in DMF (10 mL) was heated at 100° C. for 16 hours. After the reaction was completed, the mixture was cooled to room temperature. The resulting solution was diluted with water (80 mL) and extracted with EtOAc (50 mL×3). The combined organic layers were washed with brine, dried over Na₂SO₄, concentrated under vacuum. The residue was purified by flash column chromatography on silica gel (PE/EtOAc=3/1) to give methyl 3-((6-fluoro-2-methylpyridin-3-yl)oxy)-5-methyl-6-(trifluoromethyl)pyridazine-4-carboxylate (420 mg, 61.6%) as a yellow solid. LCMS (ESI) calcd. for C₁₄H₁₂F₄N₃O₃ [M+H]⁺ m/z 346.08, found 345.90.

Step 2: 3-((6-fluoro-2-methylpyridin-3-yl)oxy)-5-methyl-6-(trifluoromethyl)pyridazine-4-carboxylic acid: To a solution of methyl 3-((6-fluoro-2-methylpyridin-3-yl)oxy)-5-methyl-6-(trifluoromethyl)pyridazine-4-carboxylate (350 mg, 1.01 mmol) in THF/H₂O (1/1, 10 mL) was added LiOH (242 mg, 10.11 mmol) at room temperature. The mixture was stirred at room temperature for 16 hours. After the reaction was completed, the mixture was concentrated to remove most THE. The aqueous phase was adjusted to pH=3-4 with 1N HCl then extracted with EtOAc (30 mL×3). The combined organic layers were washed with brine, dried with Na₂SO₄, concentrated under reduced pressure to give 3-((6-fluoro-2-methylpyridin-3-yl)oxy)-5-methyl-6-(trifluoromethyl)pyridazine-4-carboxylic acid (270 mg, 80.5%) as a yellow solid. LCMS (ESI) calcd. for C₁₃H₁₀F₄N₃O₃ [M+H]⁺ m/z 332.07, found 331.90.

### Intermediate 4

### 5-bromo-2-(4-cyano-2-methoxyphenoxy)-4-methylnicotinic acid

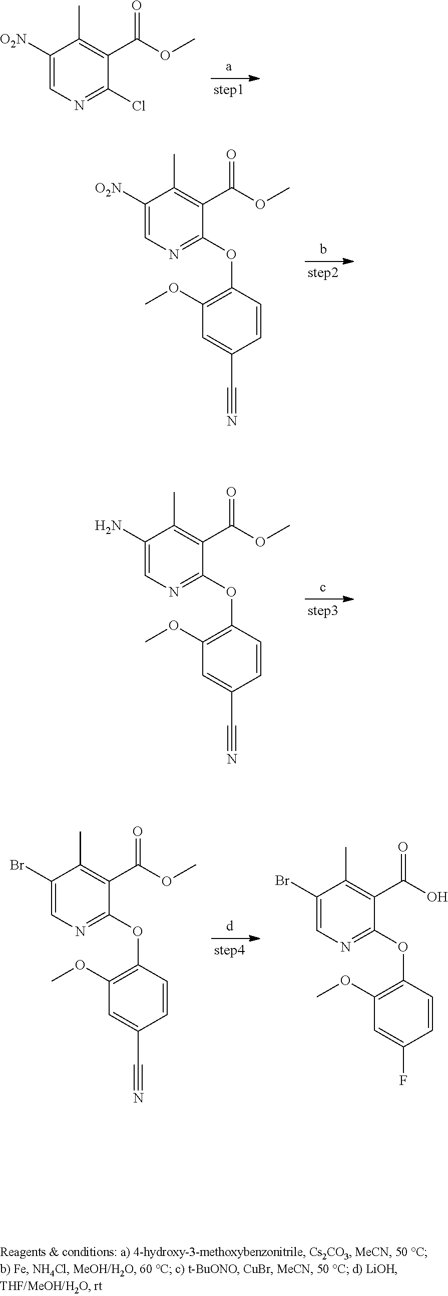 [SMILES: COC(=O)c1c(Cl)ncc([N+](=O)[O-])c1C ; COC(=O)c1c(Oc2ccc(C#N)cc2OC)ncc([N+](=O)[O-])c1C ; COC(=O)c1c(Oc2ccc(C#N)cc2OC)ncc(N)c1C ; COC(=O)c1c(Oc2ccc(C#N)cc2OC)ncc(Br)c1C ; COc1cc(F)ccc1Oc1ncc(Br)c(C)c1C(=O)O]

Step 1: methyl 2-(4-cyano-2-methoxyphenoxy)-4-methyl-5-nitronicotinate: To a solution of methyl 2-chloro-4-methyl-5-nitronicotinate (3 g, 13 mmol) and 4-hydroxy-3-methoxybenzonitrile (2.04 g, 13.7 mmol) in MeCN (30 mL) was added Cs₂CO₃ (6.36 g, 19.5 mmol). The mixture was heated at 50° C. for 1 hour. After the reaction was completed, the mixture concentrated under vacuum and directly purified by flash column chromatography on silica gel (PE/EtOAc=4/1 to 3/1) to provide methyl 2-(4-cyano-2-methoxyphenoxy)-4-methyl-5-nitronicotinate (2.5 g, 56%) as a light-yellow solid. LCMS (ESI) calcd. for C₁₆H₁₄N₃O₆ [M+H]⁺ m/z 344.09, found 343.95.

Step 2: methyl 5-amino-2-(4-cyano-2-methoxyphenoxy)-4-methylnicotinate: A solution of methyl 2-(4-cyano-2-methoxyphenoxy)-4-methyl-5-nitronicotinate (2.5 g, 7.3 mmol) in MeOH (30 mL) and water (10 mL) was added NH₄Cl (2.71 g, 51.1 mmol), Fe (2.04 g, 36.5 mmol). The mixture was heated to 60° C. for 1 hour. After the reaction was completed, the mixture was filtered through celite. The filtrate was diluted with water (100 mL) and extracted with EtOAc (50 mL×3). The combined organic phases were washed with brine, dried over sodium sulfate, concentrated under vacuum to provide methyl 5-amino-2-(4-cyano-2-methoxyphenoxy)-4-methylnicotinate (2 g, 87.7%) as a yellow solid. LCMS (ESI) calcd. for C₁₆H₁₆N₃O₄ [M+H]⁺ m/z 314.11, found 313.90.

Step 3: methyl 5-bromo-2-(4-cyano-2-methoxyphenoxy)-4-methylnicotinate: To a solution of methyl 5-amino-2-(4-cyano-2-methoxyphenoxy)-4-methylnicotinate (1 g, 3.2 mmol) and CuBr (458 mg, 3.2 mmol) in MeCN (20 mL) was added t-BuONO (391 mg, 3.8 mmol) dropwise at room temperature. The reaction solution was heated at 50° C. for 1 hour. After the reaction was completed, the resulting solution was diluted with water (20 mL) and extracted with EtOAc (30 mL×3). The combined organic phases were washed with brine, dried over sodium sulfate, concentrated under vacuum. The residue was purified by flash column chromatography on silica gel (PE/EtOAc=10/1) to give methyl 5-bromo-2-(4-cyano-2-methoxyphenoxy)-4-methylnicotinate (500 mg, 41%) as a yellow solid. LCMS (ESI) calcd. for C₁₆H₁₄BrN₂O₄ [M+H]+m/z 377.01, found 377.05.

Step 4: 5-bromo-2-(4-cyano-2-methoxyphenoxy)-4-methylnicotinic acid: A solution of methyl 5-bromo-2-(4-cyano-2-methoxyphenoxy)-4-methylnicotinate (500 mg, 1.33 mmol) in THF (3 mL), MeOH (3 mL) and H₂O (3 mL) was added LiOH·H₂O (336 mg, 8 mmol) at room temperature. The reaction mixture was stirred at room temperature for 18 hours. After the reaction was completed, the mixture was concentrated. The residue was adjusted to pH=3-4 with 1N HCl. Then the aqueous solution was extracted with EtOAc (20 mL×3). The combined organic phases were washed with brine, dried over sodium sulfate, concentrated under vacuum to provide 5-bromo-2-(4-cyano-2-methoxyphenoxy)-4-methylnicotinic acid (420 mg, 61%) as a brown solid. LCMS (ESI) calcd. for C₁₅H₁₂BrN₂O₄ [M+H]⁺ m/z 363.00, found 362.85.

### Intermediate 5

### 2-(4-cyano-2-methoxyphenoxy)-4-methyl-5-(trifluoromethyl)nicotinic acid

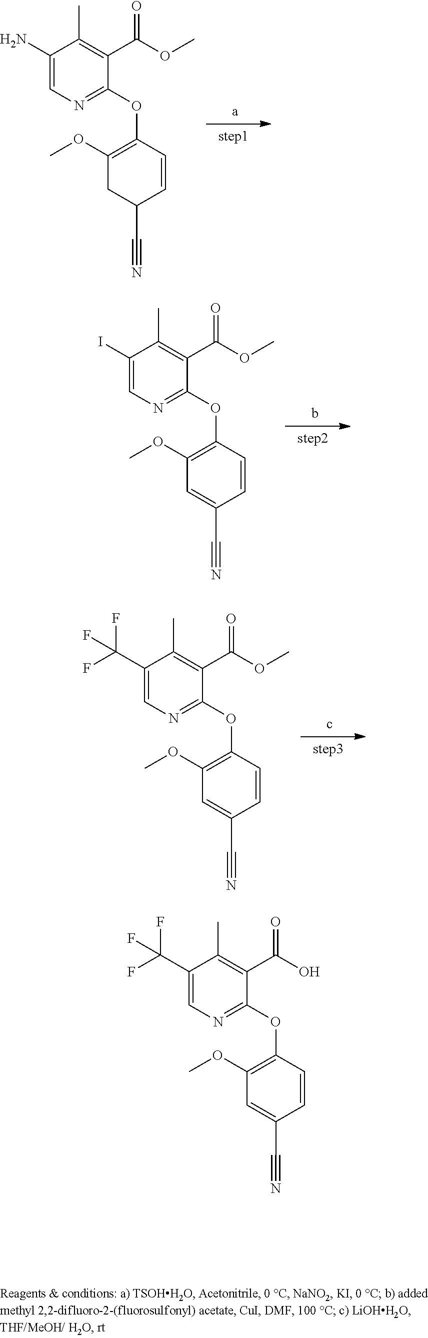

Step 1: methyl 2-(4-cyano-2-methoxyphenoxy)-5-iodo-4-methylnicotinate: To a solution of TsOH H₂O (691 mg, 3.64 mmol) and methyl 5-amino-2-(4-cyano-2-methoxyphenoxy)-4-methylnicotinate (380 mg, 1.21 mmol) in acetonitrile (12 mL) stirred air at 0° C. was added a solution of NaNO₂ (167 mg, 2.43 mmol) and potassium iodide (503 mg, 3.03 mmol) in H₂O (1.8 mL). The reaction mixture was stirred at room temperature for 2.5 h. After the reaction was completed. The mixture was quenched with water (10 mL), adjust pH (9-10) with sodium bicarbonate (20 mL) and sodium thiosulfate (20 mL). The aqueous solution was extracted with EtOAc (30 mL×3). The combined organic layers were washed with brine, dried over sodium sulfate, concentrated under vacuum. The residue was purified by flash column chromatography on silica gel (PE/EtOAc=10/1 to 1/1) to provide methyl 2-(4-cyano-2-methoxyphenoxy)-5-iodo-4-methylnicotinate (400 mg, 74.01%) as colorless oil. LCMS (ESI) calcd. for C₁₆H₁₄IN₂O₄ [M+H]⁺ m/z 425.00, found 424.95.

Step 2: methyl 2-(4-cyano-2-methoxyphenoxy)-4-methyl-5-(trifluoromethyl)nicotinate: To a stirred solution of methyl 2-(4-cyano-2-methoxyphenoxy)-5-iodo-4-methylnicotinate (380 mg, 0.89 mmol) and copper(I) iodide (341 mg, 1.79 mmol) in DMF (40 mL) was added methyl 2,2-difluoro-2-(fluorosulfonyl) acetate (860 mg, 4.48 mmol) dropwise at room temperature under an atmosphere of N2. The mixture was heated at 120° C. for 6 hours. After the reaction was completed, the resulting solution was diluted with water (60 mL) and extracted with EtOAc (30 mL×3). The combined organic phases were washed with brine, dried over sodium sulfate, concentrated under vacuum. The residue was purified by flash column chromatography on silica gel (PE/EtOAc=10/1 to 1/1) to give methyl 2-(4-cyano-2-methoxyphenoxy)-4-methyl-5-(trifluoromethyl)nicotinate (300 mg, 91.46%). LCMS (ESI) calcd. for C₁₇H₁₄F₃N₂O₄ [M+H]⁺ m/z 367.09, found 367.05.

Step 3: 2-(4-cyano-2-methoxyphenoxy)-4-methyl-5-(trifluoromethyl)nicotinic acid: A solution of methyl 2-(4-cyano-2-methoxyphenoxy)-4-methyl-5-(trifluoromethyl)nicotinate (300 mg, 0.82 mmol) in THF (2 mL), MeOH (2 mL) and H₂O (2 mL) was added LiOH·H₂O (207 mg, 4.93 mmol) at room temperature. The reaction mixture was stirred at room temperature for 18 hours. After the reaction was completed, the mixture was concentrated. The residue was adjusted to pH=3-4 with aqueous HCl (1M). Then the aqueous solution was extracted with EtOAc (20 mL×3). The combined organic layers were washed with brine, dried over sodium sulfate, concentrated under vacuum to provide 2-(4-cyano-2-methoxyphenoxy)-4-methyl-5-(trifluoromethyl)nicotinic acid (140 mg, 48.53%) as a white solid. LCMS (ESI) calcd. for C₁₆H₁₂F₃N₂O₄ [M+H]⁺ m/z 353.08, found 353.05.

### Intermediate 6

### 4-(4-cyano-2-methylphenoxy)-6-(trifluoromethyl)pyridazine-3-carboxylic acid

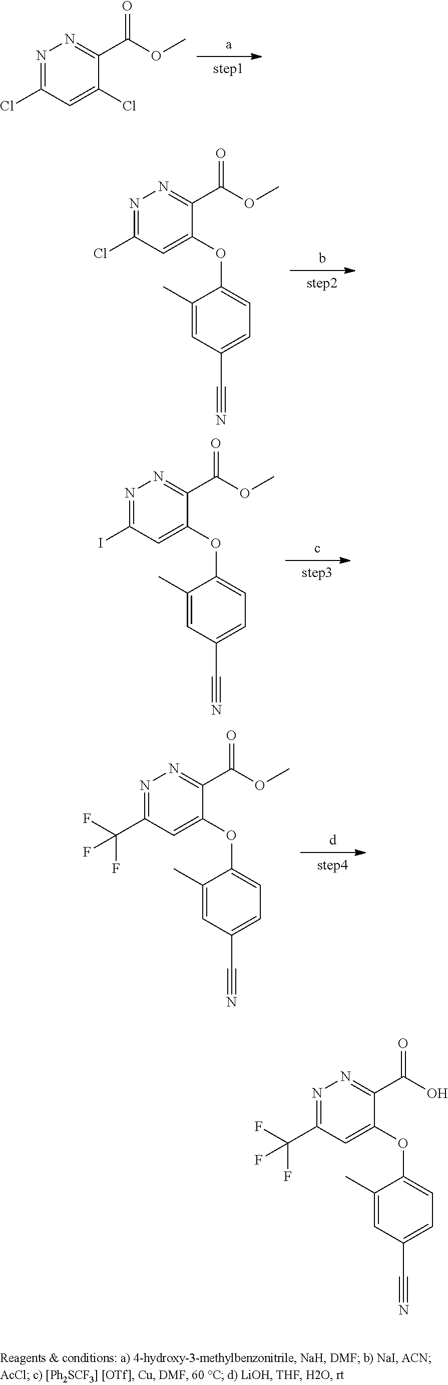

Step 1: To a solution of 4-hydroxy-3-methylbenzonitrile (1.28 g, 9.66 mmol) in DMF (10 mL) was added NaH (348 mg, 14.49 mmol, 60%). The mixture was stirred at room temperature for 30 minutes then added dropwise to a solution of methyl 4,6-dichloropyridazine-3-carboxylate (2.0 g, 9.66 mmol) in DMF (5 mL). The mixture was stirred at room temperature for 1 hour. The mixture was quenched with water (60 mL) and extracted with EtOAc (40 mL×3). The combine organic phases were washed with brine, dried over sodium sulfate, concentrated under vacuum. The residue was purified by flash column chromatography on silica gel (PE/EtOAc=2/1) to provide methyl 6-chloro-4-(4-cyano-2-methylphenoxy)pyridazine-3-carboxylate (1850 mg, 63.05%) as a yellow solid. LCMS (ESI) calcd. for C₁₄H₁₁ClN₃O₃ [M+H]⁺ m/z 304.05, found 304.10.

Step 2: To a solution of methyl 6-chloro-4-(4-cyano-2-methylphenoxy)pyridazine-3-carboxylate (1000 mg, 3.29 mmol) in MeCN (10 mL) was added NaI (4939 mg, 32.92 mmol) at 0° C. Then a solution of AcCl (568 mg, 7.24 mmol) in MeCN (5 mL) was added dropwise to the mixture at 0° C. The mixture was stirred at room temperature for 6 hours. The mixture was quenched with water (30 mL) and extracted with EtOAc (30 mL×3). The combine organic phases were washed with brine, dried over sodium sulfate, concentrated under vacuum. The residue was purified by flash column chromatography on silica gel (PE/EtOAc=2/1) to give methyl 4-(4-cyano-2-methylphenoxy)-6-iodopyridazine-3-carboxylate (800 mg, 61.48%) as a yellow solid. LCMS (ESI) calcd. for C₁₄H₁₁IN₃O₃ [M+H]⁺ m/z 395.98, found 396.15.

Step 3: To a solution of methyl 4-(4-cyano-2-methylphenoxy)-6-iodopyridazine-3-carboxylate (800 mg, 2.02 mmol) and Cu (386 mg, 6.07 mmol) in DMF (10 mL) was added [Ph₂SCF₃][OTf]⁻ (1473 mg, 3.64 mmol). The mixture was heated at 60° C. for 16 hours. Then the mixture was filtered through celite. The filtrate was diluted with water (30 mL) and extracted with EtOAc (30 mL×3). The combine organic phases were washed with brine, dried over sodium sulfate, concentrated under vacuum. The residue was purified by flash column chromatography on silica gel (PE/EtOAc=5/1) to provide methyl 4-(4-cyano-2-methylphenoxy)-6-(trifluoromethyl)pyridazine-3-carboxylate (420 mg, 55.36%) as a yellow solid. LCMS (ESI) calcd. for C₁₅H₁₁F₃N₃O₃ [M+H]⁺ m/z 338.07, found 338.15.

Step 4: To a solution of methyl 4-(4-cyano-2-methylphenoxy)-6-(trifluoromethyl)pyridazine-3-carboxylate (420 mg, 1.25 mmol) in THF/H₂O (1/1, 10 mL) was added LiOH (522 mg, 12.5 mmol) at room temperature. The mixture was stirred at room temperature for 2 hours. The aqueous phase was adjusted to pH=3-4 with 1N HCl and extracted with EtOAc (20 mL×3). The combined organic phases were washed with brine, dried with Na₂SO₄, and concentrated under reduced pressure to give 4-(4-cyano-2-methylphenoxy)-6-(trifluoromethyl)pyridazine-3-carboxylic acid (380 mg, 94.40% yield) as a yellow solid. LCMS (ESI) calcd. for C₁₄H₉F₃N₃O₃ [M+H]⁺ m/z 324.06, found 324.10.

### Intermediate 7

### 4-(3,4-difluoro-2-methoxyphenoxy)-6-(trifluoromethyl)pyridazine-3-carboxylic acid

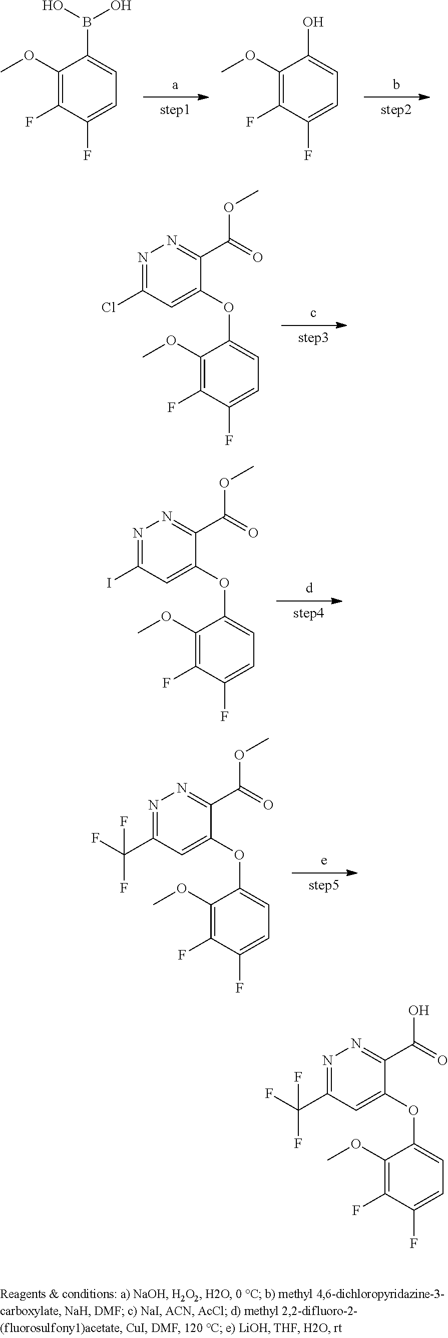

Step 1: To a solution of (3,4-difluoro-2-methoxyphenyl)boronic acid (2 g, 10.64 mmol) in THF (20 mL) was added NaOH (850 mg, 21.28 mmol), H₂O (5 mL) and H₂O₂ (1 mL, 30%) at 0° C. The mixture was stirred at room temperature for 1 h. The mixture was adjusted to pH=3-4 with 1N HCl and extracted with EtOAc (20 mL×3). The combine organic phases were washed with brine, dried over sodium sulfate, concentrated under vacuum. The residue was purified by flash column chromatography on silica gel (PE/EtOAc=3/1) to provide 3,4-difluoro-2-methoxyphenol (1.2 g, 70.59%) as a yellow solid. ¹H NMR (400 MHz, CDCl₃-d₆, ppm) δ 6.74-6.61 (m, 2H), 4.81 (s, 1H), 3.85 (s, 3H).

Step 2: To a solution of 3,4-difluoro-2-methoxyphenol (1.2 g, 7.49 mmol) in DMF (10 mL) was added NaH (0.36 g, 8.99 mmol, 60%). The mixture was stirred at room temperature for 30 minutes. Then the mixture was added dropwise to a solution of methyl 4,6-dichloropyridazine-3-carboxylate (1.54 g, 7.49 mmol) in DMF (5 mL). The mixture was stirred at room temperature for 1 h. LCMS showed the reaction was completed. The mixture was quenched with water (60 mL) and extracted with EtOAc (40 mL×3). The combine organic phases were washed with brine, dried over sodium sulfate, concentrated under vacuum. The residue was purified by flash column chromatography on silica gel (eluting with PE/EtOAc=2/1) to provide methyl 6-chloro-4-(3,4-difluoro-2-methoxyphenoxy)pyridazine-3-carboxylate (0.8 g, 32.3%) as yellow solid. LCMS (ESI) calcd. for C₁₃H₁₀ClF₂N₂O₄ [M+H]⁺ m/z 331.03, found 330.90.

Step 3: To a solution of methyl 6-chloro-4-(3,4-difluoro-2-methoxyphenoxy)pyridazine-3-carboxylate (800 mg, 2.42 mmol) in MeCN (10 mL) was added NaI (3640 mg, 24.24 mmol) at 0° C. Then a solution of AcCl (415 mg, 5.32 mmol) in MeCN (5 mL) was added dropwise to the mixture at 0° C. The mixture was stirred at room temperature for 3 h. LCMS showed the reaction was completed. The mixture was quenched with water (30 mL) and extracted with EtOAc (30 mL×3). The combine organic phases were washed with brine, dried over sodium sulfate, concentrated under vacuum. The residue was purified by flash column chromatography on silica gel (eluting PE/EtOAc=2/1) to provide methyl 4-(3,4-difluoro-2-methoxyphenoxy)-6-iodopyridazine-3-carboxylate (800 mg, 78.43%) as a yellow solid. LCMS (ESI) calcd. for C₁₃H₁₀F₂IN₂O₄ [M+H]⁺ m/z 422.97, found 422.85.

Step 4: To a solution of methyl 4-(3,4-difluoro-2-methoxyphenoxy)-6-iodopyridazine-3-carboxylate (800 mg, 1.89 mmol) and CuI (720 mg, 3.79 mmol) in DMF (10 mL) was added methyl 2,2-difluoro-2-(fluorosulfonyl)acetate (2912 mg, 15.17 mmol). The mixture was heated at 120° C. for 2 hours. LCMS showed the rection was completed. Then the mixture was filtered through celite. The filtrate was diluted with water (30 mL) and extracted with EtOAc (30 mL×3). The combine organic phases were washed with brine, dried over sodium sulfate, concentrated under vacuum. The residue was purified by flash column chromatography on silica gel (PE/EtOAc=5/1) to provide methyl 4-(3,4-difluoro-2-methoxyphenoxy)-6-(trifluoromethyl)pyridazine-3-carboxylate (360 mg, 52.17%) as a yellow solid. LCMS (ESI) calcd. for C₁₄H₁₀F₅N₂O₄ [M+H]⁺ m/z 365.06, found 365.15.

Step 5: To a solution of methyl 4-(3,4-difluoro-2-methoxyphenoxy)-6-(trifluoromethyl)pyridazine-3-carboxylate (310 mg, 0.85 mmol) in THF/H₂O (1/1, 8 mL) was added LiOH (204 mg, 8.52 mmol) at 0° C. The mixture was stirred at 0° C. for 1 hours. After the reaction was completed, the mixture was concentrated to remove the THF. The aqueous phase was adjusted to pH=3-4 with 1N HCl and extracted with EtOAc (20 mL×3). The combined organic phases were washed with brine, dried with Na₂SO₄, and concentrated under reduced pressure to give 4-(3,4-difluoro-2-methoxyphenoxy)-6-(trifluoromethyl)pyridazine-3-carboxylic acid (220 mg, 73.83%) as a yellow solid. LCMS (ESI) calcd. for C₁₃H₈F₅N₂O₄ [M+H]⁺ m/z 351.04, found 350.90.

### Intermediate 8

### 3-bromo-N,N-bis(2,4-dimethoxybenzyl)benzenesulfonamide

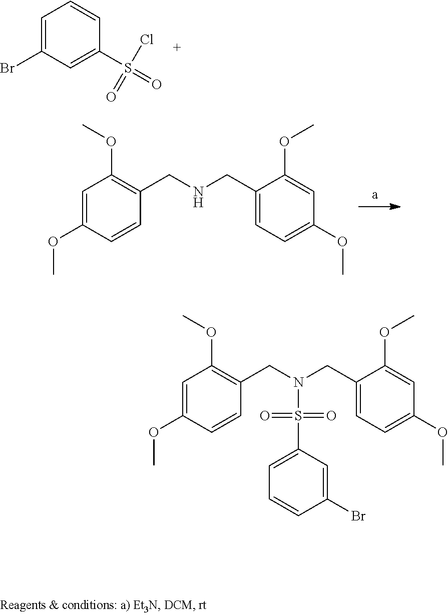

A solution of bis(2,4-dimethoxybenzyl)amine (1.00 g, 3.20 mmol), 3-bromobenzenesulfonyl chloride (0.86 g, 3.36 mmol) and triethylamine (0.97 g, 9.60 mmol) in DCM (10 mL) was stirred at room temperature for 3 hours. LCMS showed the reaction was completed. The mixture was diluted with water (30 mL) and extracted with DCM (100 mL×3). The combined organic layers were washed with brine, dried over sodium sulfate, concentrated under vacuum. The residue was purified by flash column chromatography on silica gel (PE/EtOAc=10/1 to 5/1) to provide 3-bromo-N,N-bis(2,4-dimethoxybenzyl) benzenesulfonamide (1.4 g, 83.33%) as colorless oil. LCMS (ESI) calcd. for C₂₄H₂₆BrNO₆SNa [M+Na]m/z 560.06, found 560.05.

### Intermediate 9

### tert-butyl ((3-bromophenyl)(3-((tert-butoxycarbonyl)amino)-3-ethylpentyl)(oxo)-16-sulfaneylidene)carbamate

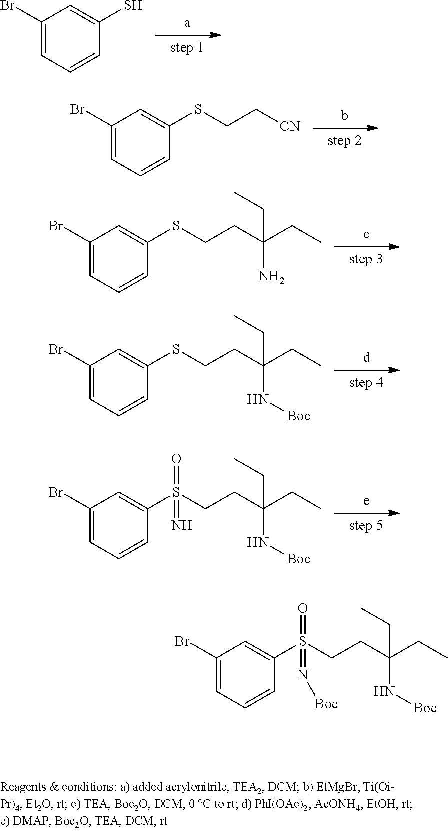

Step 1. 3-((3-bromophenyl)thio)propanenitrile: A mixture of 3-bromobenzenethiol (4 g, 0.021 mol), acrylonitrile (1.24 g, 0.023 mol) and TEA (6.44 g, 0.063 mol) in DCM (40 mL) was stirred at room temperature for 3 hours. After the reaction was completed, the resulting solution was diluted with water (25 mL) and extracted with EtOAc (25 mL×3). The combined organic phases were washed with brine, dried over Na₂SO₄, concentrated under vacuum. The residue was purified by flash column chromatography on silica gel (PE/EtOAc=20/1) to provide 3-((3-bromophenyl)thio)propanenitrile (4.6 g, 84.91% yield) as a colorless oil. ¹H NMR (400 MHz, CDCl₃, ppm) δ 7.55 (t, J=1.8 Hz, 1H), 7.42 (dd, J=7.9, 0.8 Hz, 1H), 7.36-7.31 (m, 1H), 7.21 (t, J=7.9 Hz, 1H), 3.15 (t, J=7.2 Hz, 2H), 2.62 (t, J=7.3 Hz, 2H).

Step 2: 1-((3-bromophenyl)thio)-3-ethylpentan-3-amine: A mixture of 3-((3-bromophenyl)thio)propanenitrile (3 g, 0.012 mol) in Et₂O (100 mL) under N2 at room temperature was added Titanium tetraisopropanolate (3.88 g, 0.014 mol) and ethylmagnesium bromide (5 mL, 1 mol/L in THF). The reaction mixture was stirred at room temperature for 2 hours. After the reaction was completed, the resulting solution was diluted with 10% hydroxide sodium solution (100 mL) and extracted with DCM (50 mL×3). The combined organic phases were washed with brine, dried over Na₂SO₄, concentrated under vacuum. The residue was purified by flash column chromatography on silica gel (DCM/MeOH=10/1) to provide 1-((3-bromophenyl)thio)-3-ethylpentan-3-amine (1.9 g, 50.81% yield) as a colorless oil. LCMS (ESI) calcd. for C₁₃H₂₁BrNS [M+H]⁺ m/z 302.06, found 302.0.

Step 3: tert-butyl (1-((3-bromophenyl)thio)-3-ethylpentan-3-yl)carbamate: A mixture of 1-((3-bromophenyl)thio)-3-ethylpentan-3-amine (1.9 g, 6.3 mmol) and Boc₂O (4.12 g, 0.019 mol) in DCM (50 mL) was added TEA (1.91 g, 0.019 mol) at 0° C. The reaction mixture was stirred at room temperature for 16 hours. After the reaction was completed, the resulting solution was diluted with water (50 mL) and extracted with DCM (25 mL×3). The combined organic phases were washed with brine, dried over Na₂SO₄, concentrated under vacuum. The residue was purified by flash column chromatography on silica gel (PE/EtOAc=10/1) to provide tert-butyl (1-((3-bromophenyl)thio)-3-ethylpentan-3-yl)carbamate (1.5 g, 58.73% yield) as a colorless oil. LCMS (ESI) calcd. for C₁₈H₂₉BrNO₂ [M+H]⁺ m/z 402.11, found 402.1.

Step 4: tert-butyl (1-(3-bromophenylsulfonimidoyl)-3-ethylpentan-3-yl)carbamate: A mixture of tert-butyl (1-((3-bromophenyl)thio)-3-ethylpentan-3-yl)carbamate (1.5 g, 3.7 mmol), PhI(OAc)₂ (3.58 g, 0.011 mol) and NH₄OAc (0.57 g, 0.0074 mol) in EtOH (30 mL) was stirred at room temperature for 6 hours. After the reaction was completed, the resulting solution was diluted with water (50 mL) and extracted with EtOAc (25 mL×3). The combined organic phases were washed with brine, dried over Na₂SO₄, concentrated under vacuum. The residue was purified by flash column chromatography on silica gel (PE/EtOAc=1/1) to provide tert-butyl (1-(3-bromophenylsulfonimidoyl)-3-ethylpentan-3-yl)carbamate (0.95 g, 59.46% yield) as a white solid. LCMS (ESI) calcd. for C₁₈H₃₀BrN₂O₃S [M+H]⁺ m/z 435.12, found 435.0.

Step 5: tert-butyl ((3-bromophenyl)(3-((tert-butoxycarbonyl)amino)-3-ethylpentyl)(oxo)-16-sulfaneylidene)carbamate: A mixture of tert-butyl (1-(3-bromophenylsulfonimidoyl)-3-ethylpentan-3-yl)carbamate (0.95 g, 2.19 mmol), Boc₂O (2.39 g, 0.0109 mol), TEA (1.11 g, 0.0109 mol) and DMAP (26.78 mg, 0.22 mmol) in DCM (20 mL) was stirred at room temperature for 16 hours. After the reaction was completed, the resulting solution was diluted with water (100 mL) and extracted with DCM (25 mL×3). The combined organic phases were washed with brine, dried over Na₂SO₄, concentrated under vacuum. The residue was purified by flash column chromatography on silica gel (100% DCM) to provide tert-butyl ((3-bromophenyl)(3-((tert-butoxycarbonyl)amino)-3-ethylpentyl)(oxo)-l6-sulfaneylidene)carbamate (720 mg, 61.45% yield) as a yellow oil. LCMS (ESI) calcd. for C₂₃H₃₈BrN₂O₅S [M+H]⁺ m/z 533.17, found 533.1.

### Intermediate 10

### tert-butyl((3-aminophenyl)(3-((tert-butoxycarbonyl)oxy)-3-methylbutyl)(oxo)-16-sulfaneylidene)carbamate

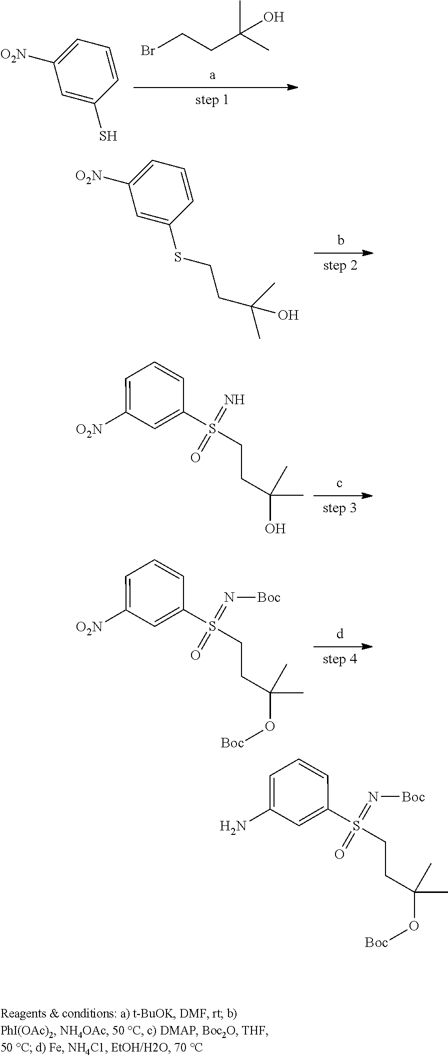

Step 1. 2-methyl-4-((3-nitrophenyl)thio)butan-2-ol: A mixture of 3-nitrobenzenethiol (600.00 mg, 3.87 mmol), 4-bromo-2-methylbutan-2-ol (775.07 mg, 4.64 mmol), t-BuOK (650.81 mg, 5.80 mmol) in DMF (10 mL) was stirred at room temperature for 16 hours. After the reaction was completed, the resulting solution was diluted with water (50 mL) and extracted with EtOAc (50 mL×3). The combined organic phases were washed with brine, dried over Na₂SO₄, concentrated under vacuum. The residue was purified by flash column chromatography on silica gel (DCM/MeOH=10/1) to provide 2-methyl-4-((3-nitrophenyl)thio)butan-2-ol (800 mg, 77.17% yield) as a yellow oil. ¹H NMR (400 MHz, DMSO-d₆, ppm) δ 8.08-7.94 (m, 2H), 7.77-7.71 (m, 1H), 7.60 (t, J=8.0 Hz, 1H), 4.42 (s, 1H), 3.17-3.05 (m, 2H), 1.75-1.62 (m, 2H), 1.15 (s, 6H).

Step 2. (3-hydroxy-3-methylbutyl)(imino)(3-nitrophenyl)-l6-sulfanone: A mixture of 2-methyl-4-((3-nitrophenyl)thio)butan-2-ol (800 mg, 3.32 mmol), PhI(OAc)₂ (3.20 g, 9.9 mmol), NH₄OAc (766.63 mg, 9.95 mol) in EtOH (20 mL) was stirred at 50° C. for 3 hours. After the reaction was completed, the resulting solution was diluted with water (60 mL) and extracted with EtOAc (60 mL×3). The combined organic phases were washed with brine, dried over Na₂SO₄, concentrated under vacuum. The residue was purified by flash column chromatography on silica gel (PE/EtOAc=2/1) to provide (3-hydroxy-3-methylbutyl)(imino)(3-nitrophenyl)-l6-sulfanone (640 mg, 63.80% yield) as a yellow oil. LCMS (ESI) calcd. for C₁₁H₁₇N₂O₄S [M+H]⁺ m/z 273.09, found 273.0.

Step 3. tert-butyl ((3-((tert-butoxycarbonyl)oxy)-3-methylbutyl)(3-nitrophenyl)(oxo)-16-sulfaneylidene)carbamate: A mixture of (3-hydroxy-3-methylbutyl)(imino)(3-nitrophenyl)-16-sulfanone (500 mg, 1.84 m mol), (Boc)₂O (1.60 g, 0.0073 mol) in THE (10 mL) was added DMAP (560.79 mg, 4.59 mmol) at 25° C. The reaction mixture was stirred at 50° C. for 16 hours. After the reaction was completed, the resulting solution was diluted with water (50 mL) and extracted with EtOAc (50 mL×3). The combined organic phases were washed with brine, dried over Na₂SO₄, concentrated under vacuum. The residue was purified by flash column chromatography on silica gel (PE/EtOAc=5/1) to provide tert-butyl ((3-((tert-butoxycarbonyl)oxy)-3-methylbutyl)(3-nitrophenyl)(oxo)-l6-sulfaneylidene)carbamate (320 mg, 25.46% yield) as a yellow oil. LCMS (ESI) calcd. for C₂₁H₃₃N₂O₈S [M+H]⁺ m/z 473.20, found 473.2.

Step 4. tert-butyl ((3-aminophenyl)(3-((tert-butoxycarbonyl)oxy)-3-methylbutyl)(oxo)-16-sulfaneylidene)carbamate. A mixture of tert-butyl ((3-((tert-butoxycarbonyl)oxy)-3-methylbutyl)(3-nitrophenyl)(oxo)-l6-sulfaneylidene)carbamate (320 mg, 0.67 mmol), NH₄Cl (108.20 mg, 2.02 mmol) in EtOH/H₂O (4/1, 10 mL) was added Fe (263.62 mg, 4.72 mmol) at 70 ° C. The reaction mixture was stirred at 70° C. for 2 hours. After the reaction was completed, the resulting mixture was filtrated through celite. The filtrate was diluted with water (60 mL) and extracted with EtOAc (60 mL×3). The combined organic phases were washed with brine, dried over Na₂SO₄, concentrated under vacuum. The residue was purified by flash column chromatography on silica gel (PE/EtOAc=2/1) to provide tert-butyl ((3-aminophenyl)(3-((tert-butoxycarbonyl)oxy)-3-methylbutyl)(oxo)-l6-sulfaneylidene)carbamate 5 (300 mg, 90.06% yield) as a yellow oil. LCMS (ESI) calcd. for C₂₁H₃₅N₂O₆S [M+H]⁺ m/z 443.22, found 443.2.

### Intermediate 11

### 3-chloro-5-((6-fluoro-2-methylpyridin-3-yl)oxy)-2-(trifluoromethyl)isonicotinic acid

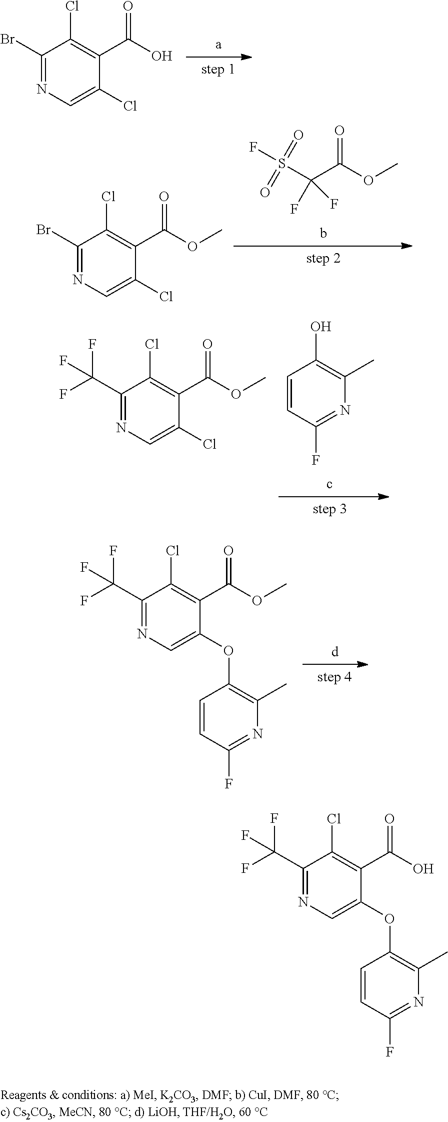

Step 1: A mixture of 2-bromo-3,5-dichloroisonicotinic acid 1 (3 g, 11.1 mmol), K₂CO₃ (4.6 g, 33.3 mmol), MeI (3.15 g, 22.2 mmol) in DMF (40 mL) was heated at 70° C. for 2 hours. The resulting solution was diluted with water (100 mL) and extracted with EtOAc (100 mL×3). The combined organic layers were washed with brine, dried over Na₂SO₄, concentrated under vacuum. The residue was purified by flash column chromatography on silica gel (PE/EtOAc=10/1 to 5/1) to give methyl 2-bromo-3,5-dichloroisonicotinate 2 (3 g, 95.5% yield) as a white solid. LCMS (ESI) calcd. for C₇H₅BrCl₂NO₂ [M+H]⁺ m/z 283.89, found 283.70.

Step 2: A solution of methyl 2-bromo-3,5-dichloroisonicotinate 2 (2 g, 7 mmol), CuI (2.76 g, 14 mmol) and methyl 2,2-difluoro-2-(fluorosulfonyl)acetate (6.72 g, 35 mmol) in DMF (25 mL) was heated at 100° C. for 5 hours under N2. LCMS showed the rection was completed. The mixture was diluted with water (80 mL) and extracted with EtOAc (3×80 mL). The combined organic layers were washed with brine, dried with Na₂SO₄, and concentrated under vacuum. The residue was purified by flash column chromatography on silica gel (PE/EtOAc=10/1 to 5/1) to give methyl 3,5-dichloro-2-(trifluoromethyl)isonicotinate 3 (1.2 g, 63% yield) as a yellow oil. LCMS (ESI) calcd. for C₈H₅C₁₂F₃NO₂ [M+H]⁺ m/z 273.97, found 273.95.

Step 3: A mixture of methyl 3,5-dichloro-2-(trifluoromethyl)isonicotinate (1.5 g, 5.5 mmol), 6-fluoro-2-methylpyridin-3-ol (420 mg, 3.3 mmol) and Cs₂CO₃ (5.38 g, 16.5 mmol) in MeCN (30 mL) was heated 80° C. for 2 hours. The mixture was cooled to room temperature, filtered through celite. The filtrate was concentrated and purified by flash column chromatography on silica gel (PE/EtOAc=10/1 to 3/1) to afford methyl 3-chloro-5-((6-fluoro-2-methylpyridin-3-yl)oxy)-2-(trifluoromethyl)isonicotinate (1.2 g, 59.9% yield) as a white oil. LCMS (ESI) calcd. for C₁₄H₁₀ClF₄N₂O₃ [M+H]⁺ m/z 365.03, found 364.95.

Step 4: To a solution of methyl 3-chloro-5-((6-fluoro-2-methylpyridin-3-yl)oxy)-2-(trifluoromethyl)isonicotinate 7 (290 mg, 0.80 mmol) in THF/1120 (1/1, 10 mL) was added LiOH (200 mg, 7.95 mmol) at 25° C. Then the mixture was heated at 60° C. for 5 hours. After the reaction was completed, the mixture was quenched by 2N HCl and adjusted pH to 3-4. Then the solution was concentrated to remove most THF. The residue was extracted with EtOAc (50 mL×3). The combined organic layers were washed with brine, dried with Na₂SO₄, and concentrated under reduced pressure to afford 3-chloro-5-((6-fluoro-2-methylpyridin-3-yl)oxy)-2-(trifluoromethyl)isonicotinic acid (190 mg, 68.1% yield) as a yellow solid. LCMS (ESI) calcd. for C₁₃H₈ClF₄N₂O₃ [M+H]⁺ m/z 351.01, found 350.95.

### Intermediate 12

### 5-((6-fluoro-2-methylpyridin-3-yl)oxy)-3-methyl-2-(trifluoromethyl)isonicotinic acid

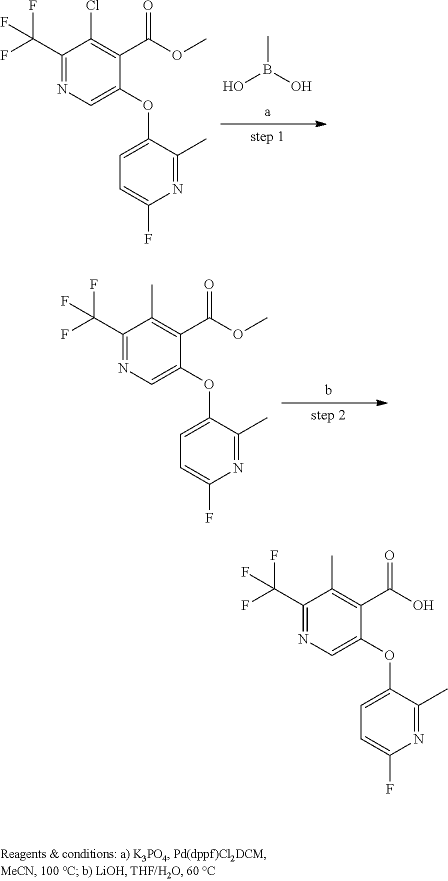

Step 1: A mixture of methyl 3-chloro-5-((6-fluoro-2-methylpyridin-3-yl)oxy)-2-(trifluoromethyl)isonicotinate (400 mg, 1.1 mmol), methylboronic acid (656.6 mg, 10.1 mmol), K₃PO₄ (1.16 g, 5.5 mmol) and Pd(dppf)Cl₂ DCM (89.6 mg, 0.11 mmol) in CH₃CN (15 mL) was heated at 100° C. for 16 hours in a sealed tube under N2. The mixture was cooled to room temperature, filtered through celite. The filtrate was concentrated and purified by flash column chromatography on silica gel (PE/EtOAc=10/1 to 3/1) to afford methyl 5-((6-fluoro-2-methylpyridin-3-yl)oxy)-3-methyl-2-(trifluoromethyl)isonicotinate (140 mg, 37% yield) as a white oil. LCMS (ESI) calcd. for C₁₅H₃F₄N₂O₃ [M+H]⁺ m/z 345.09, found 345.05.

Step 2: To a solution of methyl 5-((6-fluoro-2-methylpyridin-3-yl)oxy)-3-methyl-2-(trifluoromethyl)isonicotinate 5 (140 mg, 0.39 mmol) in THF/120 (1/1, 10 mL) was added LiOH (93 mg, 3.9 mmol) at 25° C. The mixture was heated at 80° C. for 16 hours. After the reaction was completed, the mixture was concentrated to remove most THE. The aqueous phase was adjusted to pH=3-4 with 1N HCl then extracted with EtOAc (50 mL×3). The combined organic layers were washed with brine, dried with Na₂SO₄, and concentrated under reduced pressure to afford 5-((6-fluoro-2-methylpyridin-3-yl)oxy)-3-methyl-2-(trifluoromethyl)isonicotinic acid (110 mg, 85.2% yield) as a yellow solid. LCMS (ESI) calcd. for C₁₄H₁₁F₄N₂O₃ [M+H]⁺ m/z 331.07, found 331.00.

### Intermediate 13

### tert-butyl (R)-((3-aminophenyl)(ethyl)(oxo)-l6-sulfaneylidene)carbamate

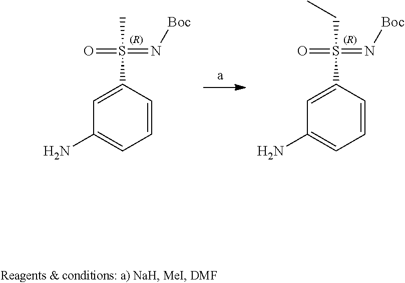

A mixture of tert-butyl (R)-aminophenyl)(methyl)(oxo)-l6-sulfaneylidene)carbamate (See: WO2022/192487; 550 mg, 2.03 mmol) in DMF (5 mL) was added NaH (60% in mineral oil, 89 mg, 2.23 mmol) 0° C. Iodomethane (346 mg, 2.23 mmol) was then added to the reaction at 0 ° C. The mixture was warmed to 25° C. and stirred for 1 hour. The reaction was monitored by LCMS. After the reaction was completed, the resulting solution was diluted with water (50 mL) and extracted with DCM (30 mL×3). The combined organic layers were washed with brine, dried over Na₂SO₄, concentrated under vacuum. The residue was purified by flash column chromatography on silica gel (PE/EtOAc=2/1 to 1/1) to provide tert-butyl (R)-((3-aminophenyl)(ethyl)(oxo)-l6-sulfaneylidene)carbamate (270 mg, 46.71% yield) as yellow oil. LCMS (ESI) calcd. for C₁₃H₂₁N₂O₃S [M+H]⁺ m/z 285.13, found 285.00.

### Intermediate 14

### tert-butyl ((3-aminophenyl)(3-((tert-butyldimethylsilyl)oxy)propyl)(oxo)-16-sulfaneylidene)carbamate

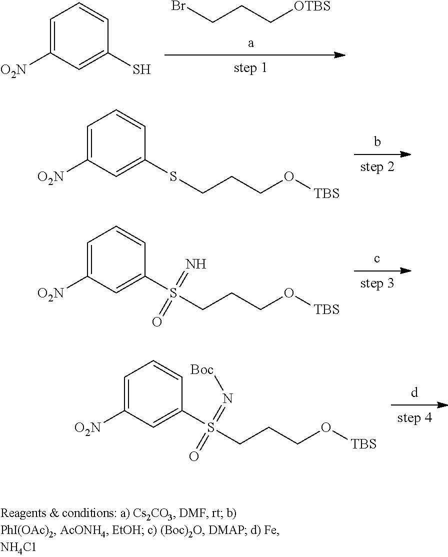

Step 1: A mixture of 3-nitrobenzenethiol (1 g, 6.4 mmol), (3-bromopropoxy)(tert-butyl)dimethylsilane (1.78 g, 7 mmol), Cs₂CO₃ (6.26 g, 19.3 mmol) in DMF (20 mL) was stirred at room temperature for 3 hours. After the reaction was completed, the resulting solution was diluted with water (50 mL) and extracted with EtOAc (50 mL×3). The combined organic phases were washed with brine, dried over Na₂SO₄, concentrated under vacuum to provide tert-butyldimethyl(3-((3-nitrophenyl)thio)propoxy)silane 3 (2 g, 95.31% yield) as a yellow oil which was used directly in next step without further purification. ¹H NMR (400 MHz, CDCl₃, ppm) δ 8.12 (t, J=1.8 Hz, 1H), 7.98 (dd, J=8.1, 1.5 Hz, 1H), 7.59 (d, J=7.9 Hz, 1H), 7.43 (t, J=8.0 Hz, 1H), 3.74 (t, J=5.8 Hz, 2H), 3.10 (t, J=7.2 Hz, 2H), 1.91-1.84 (m, 2H), 0.90 (s, 9H), 0.06 (s, 6H).

Step 2: A mixture of tert-butyldimethyl(3-((3-nitrophenyl)thio)propoxy)silane (2 g, 6.1 mmol), PhI(OAc)₂ (5.89 g, 18.3 mmol), NH₄OAc (0.94 g, 12.2 mmol) in EtOH (20 mL) was stirred at 50° C. for 6 hours. After the reaction was completed, the resulting solution was diluted with water (60 mL) and extracted with EtOAc (60 mL×3). The combined organic phases were washed with brine, dried over Na₂SO₄, concentrated under vacuum. The filtrate was concentrated and residue was purified by flash column chromatography on silica gel (PE/EtOAc=2/1) to provide (3-((tert-butyldimethylsilyl)oxy)propyl)(imino)(3-nitrophenyl)-l6-sulfanone (1.8 g, 81.97% yield) as a yellow oil. LCMS (ESI) calcd. for C₁₅H₂₇N₂O₄SSi [M+H]⁺ m/z 359.15, found 359.1.

Step 3: A mixture of (3-((tert-butyldimethylsilyl)oxy)propyl)(imino)(3-nitrophenyl)-16-sulfanone (1.68 g, 4.7 mmol), (Boc)₂O (1.03 g, 4.7 mmol) in THE (10 mL) was added DMAP (0.57 g, 4.7 mmol) at 0° C. The reaction mixture was stirred at room temperature for 3 hours. After the reaction was completed, the resulting solution was diluted with water (50 mL) and extracted with EtOAc (50 mL×3). The combined organic phases were washed with brine, dried over Na₂SO₄, concentrated under vacuum. The residue was purified by flash column chromatography on silica gel (PE/EtOAc=5/1) to provide tert-butyl ((3-((tert-butyldimethylsilyl)oxy)propyl)(3-nitrophenyl)(oxo)-l6-sulfaneylidene)carbamate (0.55 g, 25.46% yield) as a yellow oil. LCMS (ESI) calcd. for C₂₀H₃₅N₂O₆SSi [M+H]⁺ m/z 459.20, found 459.1.

Step 4: A mixture of tert-butyl ((3-((tert-butyldimethylsilyl)oxy)propyl)(3-nitrophenyl)(oxo)-l6-sulfaneylidene)carbamate (550 mg, 1.19 mmol), NH₄Cl (320.03 mg, 5.98 mmol) in MeOH/H₂O (4/1, 30 mL) was added Fe power (400.98 mg, 7.18 mmol). The reaction mixture was stirred at 60° C. for 16 hours. After the reaction was completed, the resulting mixture was filtrated through celite. The filtrate was diluted with water (60 mL) and extracted with EtOAc (60 mL×3). The combined organic phases were washed with brine, dried over Na₂SO₄, concentrated under vacuum. The residue was purified by flash column chromatography on silica gel (PE/EtOAc=2/1) to provide tert-butyl ((3-aminophenyl)(3-((tert-butyldimethylsilyl)oxy)propyl)(oxo)-16-sulfaneylidene)carbamate (350 mg, 68.08% yield) as a yellow oil. LCMS (ESI) calcd. for C₂₀H₃₇N₂O₄SSi [M+H]⁺ m/z 429.2, found 429.2.

### Intermediate 15

### di-tert-butyl ((3-bromophenyl)(methyl)-l6-sulfanediylidene)dicarbamate

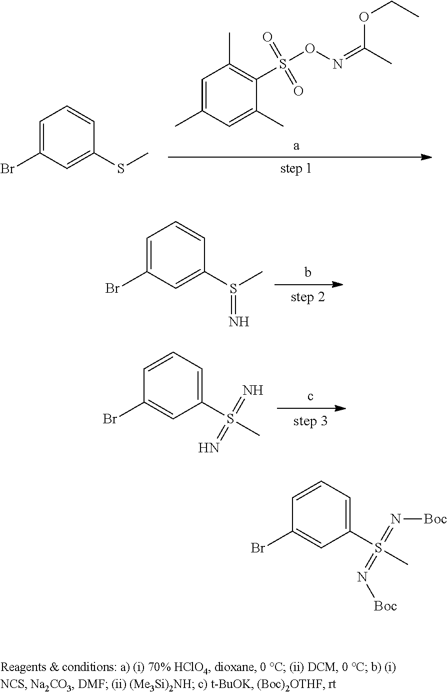

Step 1: To a solution of ethyl (Z)—N-((mesitylsulfonyl)oxy)acetimidate (4.4 g, 15.4 mmol) in dioxane (15 mL) was added HClO₄ (70%, 15 mL) dropwise at 0° C. The mixture was stirred 0° C. for 15 minutes. The mixture was quenched with cold water (30 mL) and extracted with DCM (15 mL×3). The combined organic phases were washed with brine, dried over sodium sulfate. Then a solution of (3-bromophenyl)(methyl)sulfane (2 g, 9.85 mmol) in DCM (15 mL) was added dropwise to the mixture at 0° C. The mixture was stirred at room temperature for 2 h. After the reaction was completed, ~1/2 of the DCM was evaporated, and diethyl ether (50 mL) was added to the mixture. After storing at 0° C. overnight, the crystal precipitation was collected and dried under vacuum to provide (3-bromophenyl)(methyl)-14-sulfanimine (1.5 g, 70% yield) was crystallized upon storage at 0° C. overnight. LCMS (ESI) calcd. for C₇H₉BrNS [M+H]⁺ m/z 219.96, found 219.90.

Step 2: To a solution of (3-bromophenyl)(methyl)-14-sulfanimine 2 (1.5 g, 6.88 mmol) in DMF (15 mL) was added NCS (1.1 g, 0.008 mol) and Na₂CO₃ (4.3 g, 0.041 mol) at 0° C. under N₂. The mixture was stirred at 0° C. for 15 minutes. Then (Me₃Si)₂NH (3.3 g, 0.021 mol) was added to the mixture at 0° C. The mixture was stirred at room temperature for 16 hours. The resulting solution was diluted with water (30 mL) and extracted with DCM (30 mL×3). The combined organic phases were washed with brine, dried over Na₂SO₄, concentrated under vacuum. The residue was purified by prep-HPLC (Gemini 5 um C₁₈ column, 150*21.2 mm, eluting with 5% to 30% MeCN/H₂O containing 0.05% NH₃) to provide (3-bromophenyl)(methyl)-l6-sulfanediimine (200 mg, 12.48% yield) as a white solid. LCMS (ESI) calcd. for C₇H₁₀BrN₂S [M+H]⁺ m/z 232.98, found 232.85.

Step 3: (3-bromophenyl)(methyl)-l6-sulfanediimine (200 mg, 0.862 mmol), t-BuOK (241 mg, 2.155 mmol) and Boc₂O (563 mg, 2.586 mmol) in THE (10 mL) was stirred at room temperature for 16 h. After the reaction was completed, the mixture was cooled to room temperature. The resulting solution was diluted with water (30 mL) and extracted with EtOAc (30 mL×3). The combined organic phases were washed with brine, dried over Na₂SO₄, concentrated under vacuum. The residue was purified by flash column chromatography on silica gel (PE/EtAOc=3/1) to give di-tert-butyl ((3-bromophenyl)(methyl)-l6-sulfanediylidene)dicarbamate (80 mg, 21.51% yield) as a yellow solid. LCMS (ESI) calcd. for C₁₇H₂₆BrN₂O₄S [M+H]⁺ m/z 433.08, found 433.00.

### Intermediate 16

### 5-cyclobutyl-2-((6-fluoro-2-methylpyridin-3-yl)oxy)-4-methylnicotinic acid

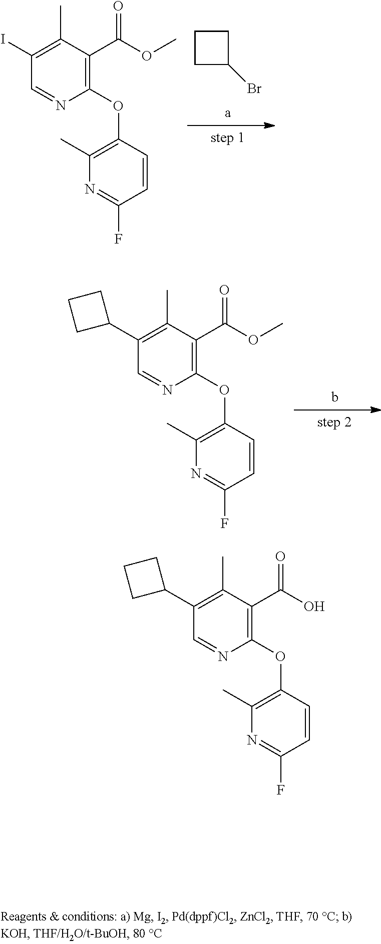

Step 1: A mixture of Mg power (3 g, 123.43 mmol) and 12 (250 mg, 0.99 mmol) in a 250 mL three-necked flask was evacuated and backfilled with N2 three times and then charged with N2. The flask was heated carefully with a hot air blower until 12 was sublimated. Then a solution of bromocyclobutane (2.25 g, 16.67 mmol) in THF (5 mL) was added intermediately via a syringe. The mixture was stirred for 10 minutes at room temperature. Then the heater and stirrer were removed. The upper layer clear solution was added dropwise into a stirred solution of methyl 2-((6-fluoro-2-methylpyridin-3-yl)oxy)-5-iodo-4-methylnicotinate (1.5 g, 3.73 mmol), ZnCl₂ (300 mg, 1.57 mmol) and Pd(dppf)Cl₂ (300 mg, 0.43 mmol) in THF (4 mL) under an atmosphere of N2. The mixture was heated to 50° C. and stirred for 1 hour. After the reaction was completed, the mixture was cooled to room temperature and the mixture was diluted with water (20 mL) and extracted with DCM (20 mL×3). The combine organic layers were washed with brine, dried over sodium sulfate, concentrated under vacuum. The residue was purified by flash column chromatography on silica gel (PE/EtOAc=10/1) to give methyl 5-cyclobutyl-2-((6-fluoro-2-methylpyridin-3-yl)oxy)-4-methylnicotinate (510 mg, 39.23% yield) as white oil. LCMS (ESI) calcd. for C₁₈H₂₀FN₂O₃ [M+H]⁺ m/z 331.15, found 330.80.

Step 2: To a solution of methyl 5-cyclobutyl-2-((6-fluoro-2-methylpyridin-3-yl)oxy)-4-methylnicotinate (510 mg, 1.54 mmol) in t-BuOH/THF/H₂O (v/v/v=1/1/1, 20 mL) was added potassium hydroxide (1.29 g, 23.11 mmol) at room temperature. The mixture was heated at 80° C. for 16 hours. The reaction was monitored by LCMS. After the reaction was completed, the mixture was cooled to room temperature, the reaction was extracted with DCM (20 mL), then the aqueous phase was adjusted to pH=3 with 1 N HCl and extracted with DCM (20 mL×3). The organic phase was dried with Na₂SO₄ and concentrated under vacuum to give 5-cyclobutyl-2-((6-fluoro-2-methylpyridin-3-yl)oxy)-4-methylnicotinic acid (410 mg, 84.01% yield) as a white solid. LCMS (ESI) calcd. for C₁₇H₁₈FN₂O₃ [M+H]⁺ m/z 317.13, found 316.90.

### Intermediate 17

### 5-cyclopentyl-2-((6-fluoro-2-methylpyridin-3-yl)oxy)-4-methylnicotinic acid

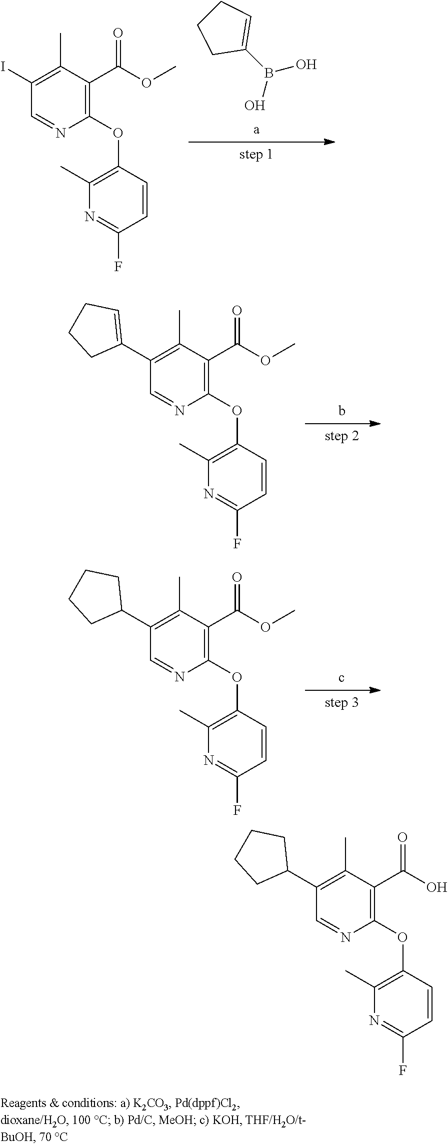

Step 1: A solution of methyl 2-((6-fluoro-2-methylpyridin-3-yl)oxy)-5-iodo-4-methylnicotinate (1 g, 2.49 mmol), cyclopent-1-en-1-ylboronic acid (1.47 g, 13.13 mmol), K₂CO₃ (1.38 g, 10.00 mmol) and Pd(dppf)Cl₂ (180 mg, 0.25 mmol) in dioxane (24 mL) and H₂O (6 mL) was heated to 100° C. and refluxed for 6 hours. LCMS showed the reaction was completed. The mixture was diluted with water (50 mL) and extracted with EtOAc (50 mL×3). The combined organic layers were washed with brine, dried over sodium sulfate, concentrated under vacuum. The residue was purified by flash column chromatography on silica gel (PE/EtOAc=10/1) to provide tert-butyl methyl 5-(cyclopent-1-en-1-yl)-2-((6-fluoro-2-methylpyridin-3-yl)oxy)-4-methylnicotinate (550 mg, 64.71% yield) as red oil. LCMS (ESI) calcd. for C₁₉H₂₀F N₂O₃ [M+H]⁺ m/z 343.15, found 343.0.

Step 2: A solution of tert-butyl methyl 5-(cyclopent-1-en-1-yl)-2-((6-fluoro-2-methylpyridin-3-yl)oxy)-4-methylnicotinate (550 mg, 1.61 mmol) in MeOH (20 mL) was added Pd/C (100 mg) and stirred at room temperature under an atmosphere of H₂. LCMS showed the reaction was completed. The mixture was filtered through celite. The filtrate was diluted with water (40 mL) and extracted with EtOAc (40 mL×3). The combined organic layers were washed with brine, dried over sodium sulfate, concentrated under vacuum. The residue was purified by flash column chromatography on silica gel (PE/EtOAc=10/1) to provide methyl 5-cyclopentyl-2-((6-fluoro-2-methylpyridin-3-yl)oxy)-4-methylnicotinate (400 mg, 72.33% yield) as colourless oil. LCMS (ESI) calcd. for C₁₉H₂₂FN₂O₃ [M+H]⁺ m/z 345.16, found 344.8.

Step 3: A solution of methyl 5-cyclopentyl-2-((6-fluoro-2-methylpyridin-3-yl)oxy)-4-methylnicotinate 7 (400 mg, 1.16 mmol) in THF/t-BuOH/H₂O (1/1/1, 21 mL) was added KOH (1.96 g, 34.85 mmol) at 0° C. The reaction mixture was heated to 80° C. and refluxed for 16 hours. After the reaction was completed, the mixture was concentrated. The residue was adjusted to pH=3-4 with aqueous HCl (1M). Then the solution was extracted with EtOAc (50 mL×3). The combined organic layers were washed with brine, dried over sodium sulfate, concentrated under vacuum to provide 5-cyclopentyl-2-((6-fluoro-2-methylpyridin-3-yl)oxy)-4-methylnicotinic acid (350 mg, 91.38% yield) as a white solid. LCMS (ESI) calcd. for C₁₈H₂₁FN₂O₃ [M+H]⁺ m/z 331.15, found 330.8.

### Intermediate 18

### 2-((6-fluoro-2-methylpyridin-3-yl)oxy)-4-methyl-5-(perfluoroethyl)nicotinic acid

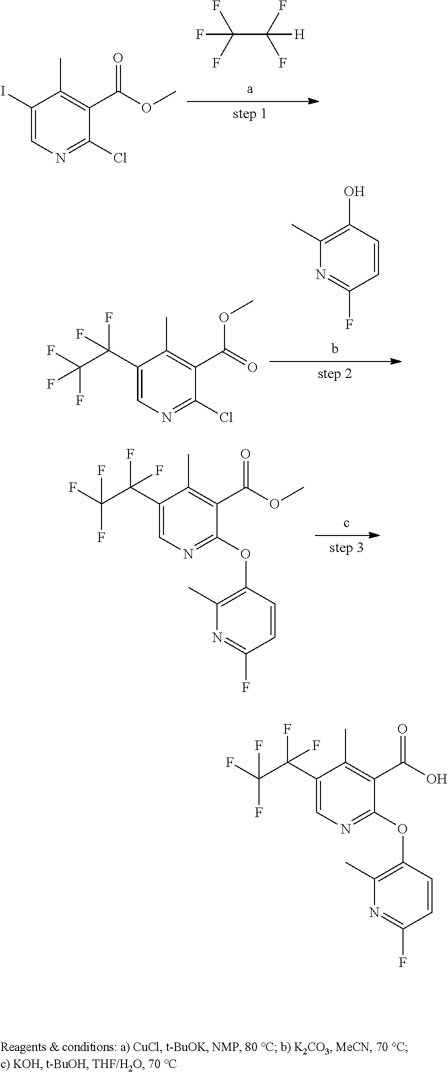

Step 1. methyl 2-chloro-4-methyl-5-(perfluoroethyl)nicotinate: In a glove-box, a three-neck 1000 ml round-bottom flask equipped with a Teflon coated magnetic stir-bar was charged with t-BuOK (34 g, 0.3 mol) and NMP (500 ml). Then CuCl (15 g, 0.15 mol) was added at agitation and the reaction mixture was vigorously stirred for 30 min at room temperature, C₂F₅H was introduced as vigorous stirring over period of 30 min. The reaction mixture was then stirred for additional 1 hour. Et₃N·3HF (0.0265 mol, 4.3 g) was added to the reaction mixture and then was stirred for another additional 1 hour. Methyl 2-chloro-5-iodo-4-methylnicotinate (0.05 mol, 16 g) was added, the reaction mixture was stirred at 80° C. overnight. The reaction was cooled to room temperature, then poured into water (3 L), filtered, the filter cake was washed with EtOAc (1 L), the filtrate was separated, the aqueous was extracted with EtOAc (500 ml, two times), combined the organic phase and washed with brine (1 L), the organic phase was dried over Na₂SO₄, evaporated in vacuo. The crude product was purified by silica gel chromatography eluting with heptane/EtOAc (100/1-20/1) to give methyl 2-chloro-4-methyl-5-(perfluoroethyl)nicotinate (4.5 g, yield: 30%) as light yellow oil. LCMS (ESI) calcd. for C₁₀H₈ClF₅NO₂ [M+H]⁻ m/z 304.0, found 303.8. ¹HNMR (400 MHz, CDCl₃) δ 8.56 (s, 1H), 4.01 (s, 3H), 2.45-2.46 (m, 3H)

Step 2: methyl 2-((6-fluoro-2-methylpyridin-3-yl)oxy)-4-methyl-5-(perfluoroethyl)nicotinate: To solution of methyl 2-chloro-4-methyl-5-(perfluoroethyl)nicotinate (3.8 g, 12.52 mmol) in MeCN (28.5 mL) was added 6-fluoro-2-methylpyridin-3-ol (1.67 g, 13.15 mmol) and K₂CO₃ (5.18 g, 37.54 mmol). The mixture was heated to 70° C. and stirred for 16 hours. After the reaction finished, the mixture was cooled to temperature and filtered, then the filtrate was concentrated under vacuum to afford the crude product (6.8 g). The crude was purified by silica gel chromatography (EtOAc:PE=1:20) to afford the methyl 2-((6-fluoro-2-methylpyridin-3-yl)oxy)-4-methyl-5-(perfluoroethyl)nicotinate (4.0 g, yield: 81.14%) as white solid. ¹H NMR (400 MHz, CDCl₃) δ 8.26 (s, 1H), 7.52-7.56 (m, 1H), 6.81-6.84 (m, 1H) 4.02 (s, 3H), 2.48 (s, 3H), 2.34 (s, 3H). LCMS (ESI) calcd. for C₁₆H₁₃F₆N₂O₃ [M+H]⁺ m/z 395.1, found 395.0.

Step 3: 2-((6-fluoro-2-methylpyridin-3-yl)oxy)-4-methyl-5-(perfluoroethyl)nicotinic acid: To a solution of methyl 2-((6-fluoro-2-methylpyridin-3-yl)oxy)-4-methyl-5-(perfluoroethyl)nicotinate (4.0 g, 10.15 mmol) in THE (16 mL) was added KOH (2.85 g, 50.73 mmol), tert-butanol (16 mL), water (16 mL). The mixture was heated to 70° C. and stirred for 24 hours. The reaction was concentrated, and residue was adjusted to pH=2-3 with HCl (1M). Then the mixture was extracted with EtOAc (30 mL×2). The organic phase was combined and washed by brine (30 mL), then dried over anhydrous Na₂SO₄. The solution was filtered and then the filtrate was concentrated under vacuum to afford the crude product. The crude was stirred in heptane (20 mL) for 1 hour then filtered and collected the filter cake to afford 2-((6-fluoro-2-methylpyridin-3-yl)oxy)-4-methyl-5-(perfluoroethyl) nicotinic acid (3.4 g, yield: 88.15%) as light brown solid. ¹H NMR (400 MHz, CDCl₃) δ 8.23 (s, 1H), 7.19-7.52 (m, 1H), 6.77-6.80 (m, 1H), 2.51 (s, 3H), 2.29 (s, 3H). LCMS (ESI) calcd. for C₁₅H₁₁F₆N₂O₃ [M+H]⁺ m/z 381.1, found 381.0.

### Intermediate 19

### 2-((6-chloro-2-methylpyridin-3-yl)oxy)-4-methyl-5-(trifluoromethyl)nicotinic acid

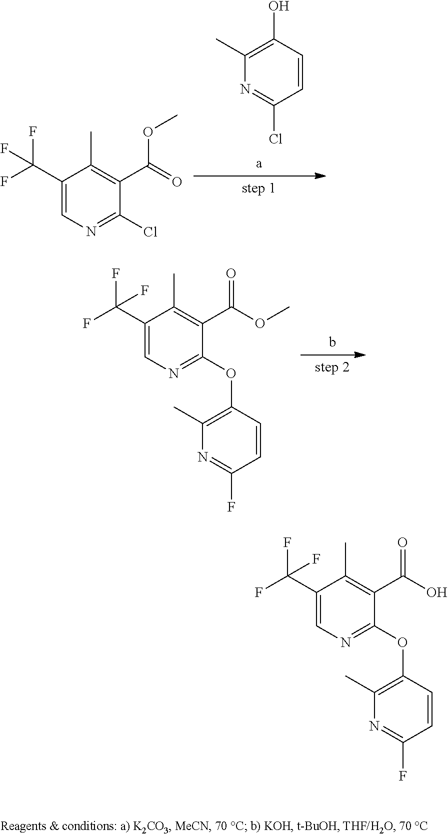

Step 1: methyl 2-((6-chloro-2-methylpyridin-3-yl)oxy)-4-methyl-5-(trifluoromethyl)nicotinate: To solution of methyl 2-chloro-4-methyl-5-(trifluoromethyl)nicotinate (6.36 g, 25.1 mmol) in MeCN (48 mL) was added 6-chloro-2-methylpyridin-3-ol (3.0 g, 20.9 mmol) and K₂CO₃ (8.65 g, 62.9 mmol). The mixture was heated to 70° C. and stirred for 16 hours. After the reaction finished, the mixture was cooled to temperature and filtered, then the filtrate was concentrated under vacuum to afford the crude product. The crude was purified by silica gel chromatography (ethyl acetate: petroleum ether 1:40 to 1:20) to afford methyl2-((6-chloro-2-methylpyridin-3-yl)oxy)-4-methyl-5-(trifluoromethyl)nicotinate (6.28 g, yield: 69.47%) as white solid. ¹H NMR (400 MHz, CDCl₃) δ 8.33 (s, 1H), 7.41-7.43 (m, 1H), 7.22-7.26 (m, 1H) 4.02 (s, 3H), 2.49 (m, 3H), 2.37 (s, 3H). LCMS (ESI) calcd. for C₁₅H₁₃ClF₃N₂O₃ [M+H]⁺ m/z 361.1, found 361.0.

Step 2: 2-((6-chloro-2-methylpyridin-3-yl)oxy)-4-methyl-5-(trifluoromethyl)nicotinic acid: To a solution of methyl2-((6-chloro-2-methylpyridin-3-yl)oxy)-4-methyl-5-(trifluoromethyl)nicotinate (2.04 g, 10.15 mmol) in t-Butanol (8 mL) and water (8 ml) was added Tetrahydrofuran (8 ml), the resulting mixture was added Potassium hydroxide (1.59 g, 28.28 mmol). The mixture was heated to 70° C. and stirred for 16 hours. The reaction was concentrated, and residue was adjusted to pH=2-3 with aqueous hydrocholoride (1M). Then the mixture was extracted with EtOAc (20 mL×2). The organic phase was combined and washed by brine (30 mL), then dried over anhydrous Na₂SO₄. The solution was filtered and then the filtrate was concentrated under vacuum to afford the crude product. The crude was stirred in heptane (20 mL) for 1 hour then filtered and collected the filter cake to afford 2-((6-chloro-2-methylpyridin-3-yl)oxy)-4-methyl-5-(trifluoromethyl)nicotinic acid (1.95 g, yield: 99%) as white solid. ¹H NMR (400 MHz, CDCl₃) δ 8.36 (s, 1H), 7.46-7.49 (m, 1H), 7.25-7.27 (m, 1H), 2.59 (s, 3H), 2.42 (s, 3H). LCMS (ESI) calcd. for C₁₄H₁₁F₃N₂O₃ [M+H]⁺ m/z 347.0, found 346.8.

### Example 1C

### (R)-2-(4-cyano-2-methoxyphenoxy)-4-methyl-5-(1-methyl-1H-pyrazol-4-yl)-N-(3-(S-methylsulfonimidoyl)phenyl)nicotinamide

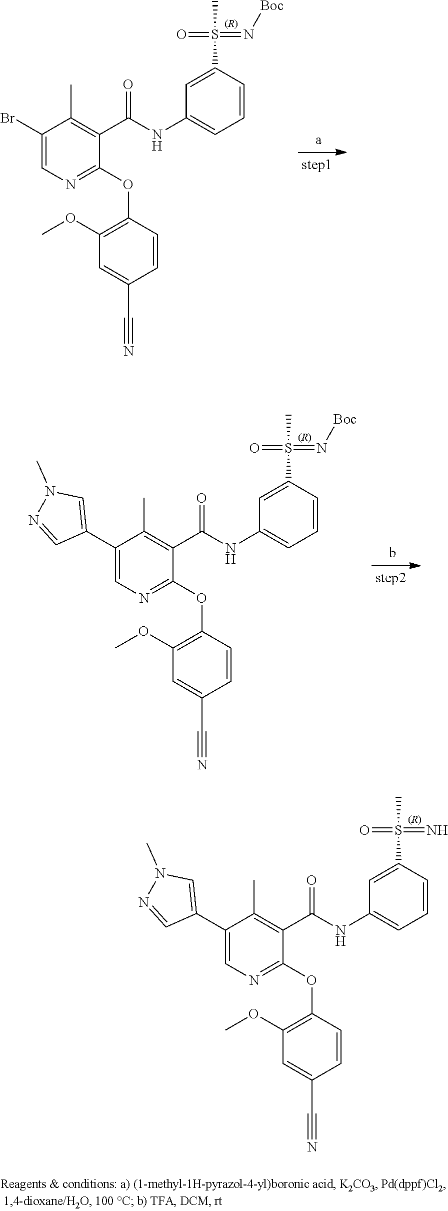

Step 1: tert-butyl (R)-((3-(2-(4-cyano-2-methoxyphenoxy)-4-methyl-5-(1-methyl-1H-pyrazol-4-yl)nicotinamido)phenyl)(methyl)(oxo)-λ⁶-sulfaneylidene)carbamate: A solution of tert-butyl (R)-((3-(5-bromo-2-(4-cyano-2-methoxyphenoxy)-4-methylnicotinamido)phenyl)(methyl)(oxo)-λ⁶-sulfaneylidene)carbamate (See: Example 20, 110 mg, 0.18 mmol), (1-methyl-1H-pyrazol-4-yl)boronic acid (27 mg, 0.21 mmol), K₂CO₃ (49 mg, 0.36 mmol) and Pd(dppf)Cl₂ (13 mg, 0.02 mmol) in dioxane (10 mL) and H₂O (2.5 mL) was heated at 100° C. for 6 hours. LCMS showed the reaction was completed. The mixture was diluted with water (20 mL) and extracted with EtOAc (20 mL×3). The combined organic layers were washed with brine, dried over sodium sulfate, concentrated under vacuum. The residue was purified by flash column chromatography on silica gel (PE/EtOAc=10/1 to 1/1) to provide tert-butyl (R)-((3-(2-(4-cyano-2-methoxyphenoxy)-4-methyl-5-(1-methyl-1H-pyrazol-4-yl)nicotinamido)phenyl)(methyl)(oxo)-λ⁶-sulfaneylidene)carbamate (70 mg, 63.64%) as yellow oil. LCMS (ESI) calcd. for C₃₁H₃₃N₆O₆S [M+H]⁺ m/z 617.22, found 617.20.

Step 2: (R)-2-(4-cyano-2-methoxyphenoxy)-4-methyl-5-(1-methyl-1H-pyrazol-4-yl)-N-(3-(S-methylsulfonimidoyl)phenyl)nicotinamide: A solution of tert-butyl (R)-((3-(2-(4-cyano-2-methoxyphenoxy)-4-methyl-5-(1-methyl-1H-pyrazol-4-yl)nicotinamido)phenyl)(methyl)(oxo)-λ⁶-sulfaneylidene)carbamate (70 mg, 0.11 mmol) in DCM (2 mL) was added TFA (0.2 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 20% to 95% MeCN/H₂O containing 0.05% NH₃H₂O) to provide (R)-2-(4-cyano-2-methoxyphenoxy)-4-methyl-5-(1-methyl-1H-pyrazol-4-yl)-N-(3-(S-methylsulfonimidoyl)phenyl)nicotinamide (9.5 mg, 16.21%) as a white solid. ¹H NMR (400 MHz, DMSO-d₆) δ 11.05 (s, 1H), 8.46 (t, J=1.8 Hz, 1H), 8.10 (s, 1H), 8.01 (s, 1H), 7.89 (d, J=8.1 Hz, 1H), 7.68 (d, J=9.4 Hz, 2H), 7.63 (d, J=1.8 Hz, 1H), 7.60 (t, J=7.9 Hz, 1H), 7.48 (dd, J=8.2, 1.8 Hz, 1H), 7.35 (d, J=8.2 Hz, 1H), 3.90 (s, 3H), 3.78 (s, 3H), 3.10 (s, 3H), 2.38 (s, 3H). LCMS (ESI) calcd. for C₂₆H₂₅N₆O₄S [M+H]⁺ m/z 517.17, found 517.10.

### Example 2C

### (R)-5-(4-(difluoromethyl)phenyl)-2-((6-fluoro-2-methylpyridin-3-yl)oxy)-4-methyl-N-(3-(S-methylsulfonimidoyl)phenyl)nicotinamide

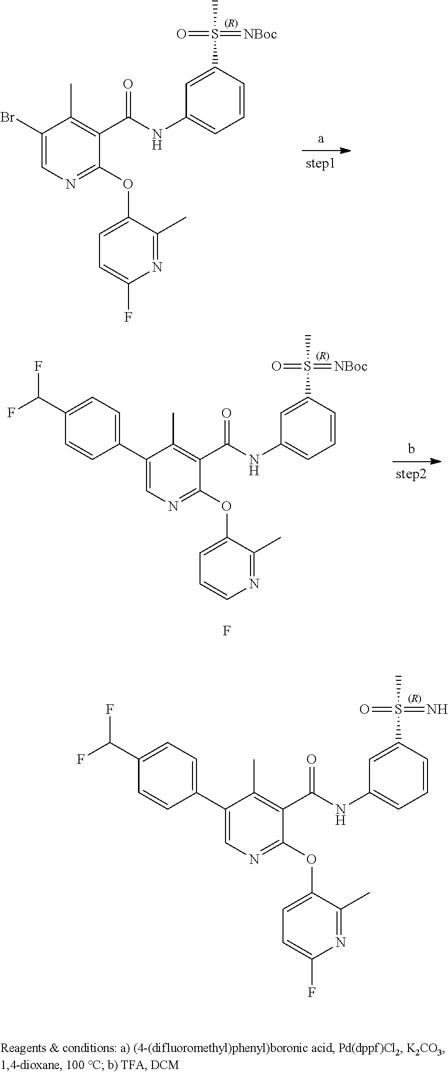

Step 1: tert-butyl (R)-((3-(5-(4-(difluoromethyl)phenyl)-2-((6-fluoro-2-methylpyridin-3-yl)oxy)-4-methylnicotinamido)phenyl)(methyl)(oxo)-λ⁶-sulfaneylidene) carbamate: A mixture of tert-butyl (R)-((3-(5-bromo-2-((6-fluoro-2-methylpyridin-3-yl)oxy)-4-methylnicotinamido)phenyl)(methyl)(oxo)-λ⁶-sulfaneylidene)carbamate (Example 3C; 250 mg, 0.42 mmol) and (4-(difluoromethyl)phenyl)boronic acid (216 mg, 1.26 mmol) in 1,4-dioxane/H₂O (4/1, 20 mL), potassium carbonate (174 mg, 1.26 mmol) and [1,1′-Bis(diphenylphosphino)ferrocene]dichloropalladium (II) (48 mg, 0.06 mmol) was heated at 100° C. for 6 hours under an atmosphere of N2. The reaction was monitored by LCMS. After the reaction was completed, the mixture was cooled to room temperature. The resulting solution was diluted with water (100 mL) and extracted with EA (50 mL×3). The combine organic phases were washed with brine, dried over Na₂SO₄, concentrated under vacuum. The residue was purified by flash column chromatography on silica gel (PE/EtAOc=1/1) to give tert-butyl (R)-((3-(5-(4-(difluoromethyl)phenyl)-2-((6-fluoro-2-methylpyridin-3-yl)oxy)-4-methylnicotinamido)phenyl)(methyl)(oxo)-λ⁶-sulfaneylidene) carbamate (243 mg, 89%) as a yellow oil. LCMS (ESI) calcd. for C₃₂H₃₂F₃N₄O₅S [M+H]⁺ m/z 641.21, found 641.05.

Step 2: (R)-5-(4-(difluoromethyl)phenyl)-2-((6-fluoro-2-methylpyridin-3-yl)oxy)-4-methyl-N-(3-(S-methylsulfonimidoyl)phenyl)nicotinamide: A solution of tert-butyl (R)-((3-(5-(4-(difluoromethyl)phenyl)-2-((6-fluoro-2-methylpyridin-3-yl)oxy)-4-methylnicotinamido)phenyl)(methyl)(oxo)-λ⁶-sulfaneylidene)carbamate (243 mg, 0.38 mmol) in DCM (5 mL) was added TFA (0.5 mL) at room temperature. The reaction mixture was stirred at room temperature for 2 hours. After the reaction was completed, the solution was diluted with water (40 mL) and extracted with DCM (20 mL×3). The combine organic phases were washed with brine, dried over sodium sulfate, and concentrated under vacuum. The residue was purified by prep-HPLC (Gemini 5 um C₁₈ column, 150*21.2 mm, eluting with 45% to 95% MeCN/H₂O containing 0.1% NH₃) to obtain (R)-5-(4-(difluoromethyl)phenyl)-2-((6-fluoro-2-methylpyridin-3-yl)oxy)-4-methyl-N-(3-(S-methylsulfonimidoyl)phenyl)nicotinamide (126.5 mg, 61.7%) as a white solid. ¹H NMR (400 MHz, DMSO-d₆) δ 11.17 (s, 1H), 8.46 (t, J=1.8 Hz, 1H), 8.07 (s, 1H), 7.95-7.78 (m, 2H), 7.69 (m, 3H), 7.65-7.46 (m, 3H), 7.31-6.90 (m, 2H), 4.24 (s, 1H), 3.07 (s, 3H), 2.30 (s, 3H), 2.29 (s, 3H). LCMS (ESI) calcd. for C₂₇H₂₄F₃N₄O₃S [M+H]⁺ m/z 541.15, found 541.15.

### Example 3C

### (R)-5-(4-cyanophenyl)-2-((6-fluoro-2-methylpyridin-3-yl)oxy)-4-methyl-N-(3-(S-methylsulfonimidoyl)phenyl)nicotinamide

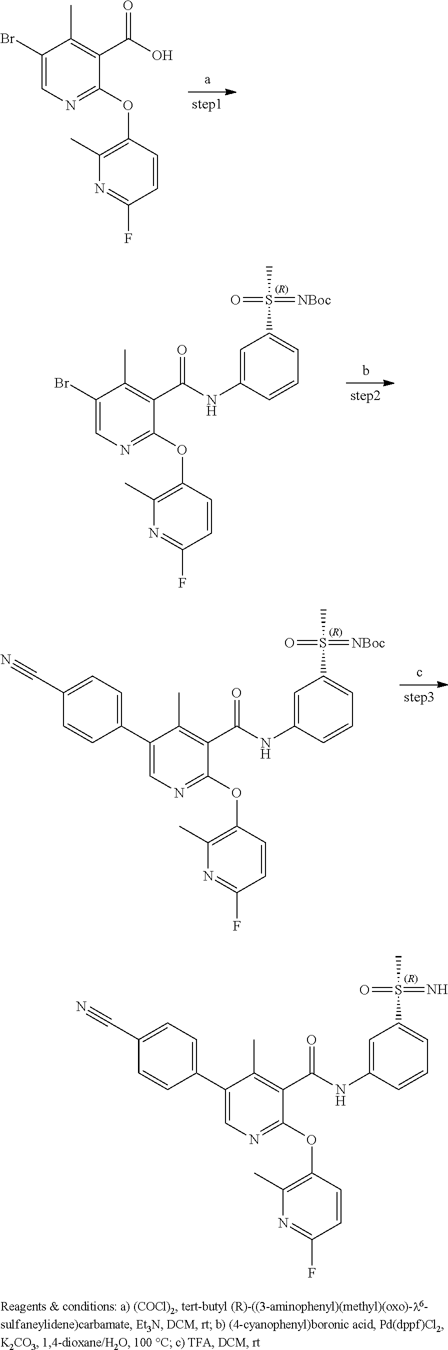

Step 1: tert-butyl (R)-((3-(5-bromo-2-((6-fluoro-2-methylpyridin-3-yl)oxy)-4-methylnicotinamido)phenyl)(methyl)(oxo)-λ⁶-sulfaneylidene)carbamate: A solution of 5-bromo-2-((6-fluoro-2-methylpyridin-3-yl)oxy)-4-methylnicotinic acid (470 mg, 1.38 mmol) in DCM (10 mL) was added DMF (one drop) and oxalyl chloride (297 mg, 2.32 mmol) at room temperature. The mixture was stirred at room temperature for 5 min. Then the solution was added to a stirred solution of tert-butyl (R)-((3-aminophenyl)(methyl)(oxo)-λ⁶-sulfaneylidene)carbamate (WO2022/192487, which is incorporated by reference in its entirety; 410 mg, 1.52 mmol) and TEA (418 mg, 4.14 mmol) in DCM (5 mL) at 0° C. The resulting mixture was stirred at 25° C. for 0.5 hour. Then the mixture was quenched with water (40 mL) and extracted with DCM (30 mL×2). The combined organic phases were washed with brine, dried over sodium sulfate, concentrated under vacuum. The residue was purified by flash column chromatography on silica gel (PE/EtOAc=3/1) to provide tert-butyl (R)-((3-(5-bromo-2-((6-fluoro-2-methylpyridin-3-yl)oxy)-4-methylnicotinamido)phenyl)(methyl)(oxo)-λ⁶-sulfaneylidene)carbamate (560 mg, 68%) as yellow solid. LCMS (ESI) calcd. for C₂₅H₂₇BrFN₄O₅S [M+H]⁺ m/z 595.09, found 594.95.

Step 2: tert-butyl (R)-((3-(5-(4-cyanophenyl)-2-((6-fluoro-2-methylpyridin-3-yl)oxy)-4-methylnicotinamido)phenyl)(methyl)(oxo)-λ⁶-sulfaneylidene)carbamate: A mixture of tert-butyl (R)-((3-(5-bromo-2-((6-fluoro-2-methylpyridin-3-yl)oxy)-4-methylnicotinamido)phenyl) (methyl)(oxo)-λ⁶-sulfaneylidene)carbamate (250 mg, 0.42 mmol) and (4-cyanophenyl)boronic acid (185 mg, 1.26 mmol) in 1,4-dioxane/H₂O (4/1, 20 mL), potassium carbonate (174 mg, 1.26 mmol) and [1,1′-Bis(diphenylphosphino)ferrocene]dichloropalladium (II) (24 mg, 0.03 mmol) was heated at 100° C. for 6 hours under an atmosphere of N2. The reaction was monitored by LCMS. After the reaction was completed, the mixture was cooled to room temperature. The resulting solution was diluted with water (50 mL) and extracted with EtOAc (50 mL×3). The combine organic phases were washed with brine, dried over Na₂SO₄, concentrated under vacuum. The residue was purified by flash column chromatography on silica gel (PE/EtAOc=1/1) to give tert-butyl (R)-((3-(5-(4-cyanophenyl)-2-((6-fluoro-2-methylpyridin-3-yl)oxy)-4-methylnicotinamido)phenyl)(methyl)(oxo)-λ⁶-sulfaneylidene)carbamate (240 mg, 93.0%) as a yellow oil. LCMS (ESI) calcd. for C₃₂H₃₁FN₅O₅S [M+H]⁺ m/z 616.21, found 616.15.

Step 3: (R)-5-(4-cyanophenyl)-2-((6-fluoro-2-methylpyridin-3-yl)oxy)-4-methyl-N-(3-(S-methylsulfonimidoyl)phenyl)nicotinamide: A solution of tert-butyl (R)-((3-(5-(4-cyanophenyl)-2-((6-fluoro-2-methylpyridin-3-yl)oxy)-4-methylnicotinamido)phenyl)(methyl)(oxo)-λ⁶-16-sulfaneylidene)carbamate (240 mg, 0.39 mmol) in DCM (5 mL) was added TFA (0.5 mL) at room temperature. The reaction mixture was stirred at room temperature for 2 hours. After the reaction was completed, the solution was diluted with water (30 mL) and extracted with DCM (20 mL×3). The combine organic phases were washed with brine, dried over sodium sulfate, and concentrated under vacuum. The residue was purified by prep-HPLC (Gemini 5 um C₁₈ column, 150*21.2 mm, eluting with 35% to 90% MeCN/H₂O containing 0.1% NH₃) to obtain (R)-5-(4-cyanophenyl)-2-((6-fluoro-2-methylpyridin-3-yl)oxy)-4-methyl-N-(3-(S-methylsulfonimidoyl)phenyl)nicotinamide (112.5 mg, 56.0%) as a white solid. ¹H NMR (400 MHz, DMSO-d₆) δ 11.18 (s, 1H), 8.46 (t, J=1.8 Hz, 1H), 8.08 (s, 1H), 7.98 (d, J=8.4 Hz, 2H), 7.92-7.79 (m, 2H), 7.72-7.56 (m, 4H), 7.10 (dd, J=8.6, 3.3 Hz, 1H), 4.24 (s, 1H), 3.07 (s, 3H), 2.30 (s, 3H), 2.28 (s, 3H). LCMS (ESI) calcd. for C₂₇H₂₃FN₅O₃S [M+H]⁺ m/z 516.15, found 516.05.

### Example 4C

### (R)-5-(2,3-dihydrobenzo[b][1,4]dioxin-6-yl)-2-((6-fluoro-2-methylpyridin-3-yl)oxy)-4-methyl-N-(3-(S-methylsulfonimidoyl)phenyl)nicotinamide

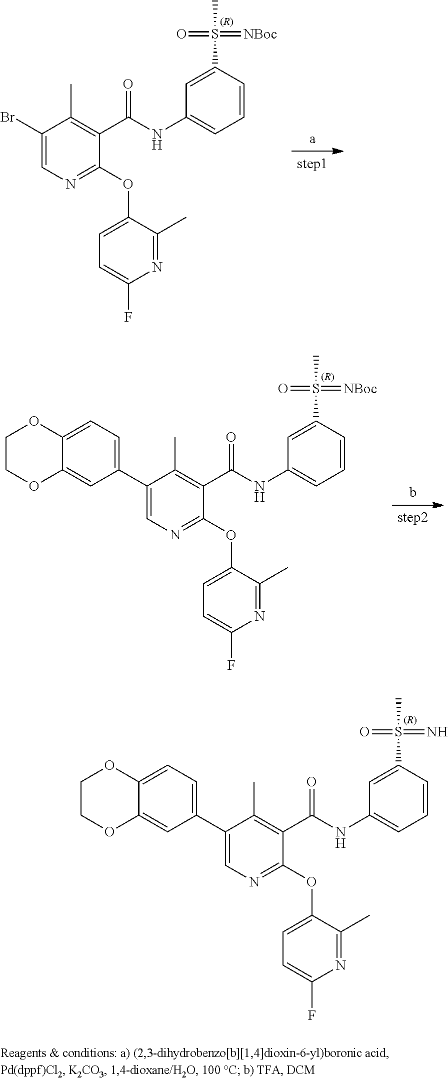

Step 1: tert-butyl (R)-((3-(5-(2,3-dihydrobenzo[b][1,4]dioxin-6-yl)-2-((6-fluoro-2-methylpyridin-3-yl)oxy)-4-methylnicotinamido)phenyl)(methyl)(oxo)-16-sulfaneylidene)carbamate: A mixture of tert-butyl (R)-((3-(5-bromo-2-((6-fluoro-2-methylpyridin-3-yl)oxy)-4-methylnicotinamido)phenyl)(methyl)(oxo)-λ⁶-sulfaneylidene)carbamate (150 mg, 0.25 mmol), (2,3-dihydrobenzo[b][1,4]dioxin-6-yl)boronic acid (91 mg, 0.51 mmol), potassium carbonate (104.8 mg, 0.76 mmol) and Pd(dppf)Cl₂ (37 mg, 0.05 mmol) in 1,4-dioxane/H₂O (4/1, 10 mL) was heated at 100° C. for 5 hours under an atmosphere of N2. After the reaction was completed, the mixture was cooled to room temperature. The resulting solution was diluted with water (30 mL) and extracted with EtOAc (30 mL×3). The combine organic phases were washed with brine, dried over Na₂SO₄, concentrated under vacuum. The residue was purified by flash column chromatography on silica gel (PE/EtAOc=1/1) to give tert-butyl (R)-((3-(5-(2,3-dihydrobenzo[b][1,4]dioxin-6-yl)-2-((6-fluoro-2-methylpyridin-3-yl)oxy)-4-methylnicotinamido)phenyl)(methyl)(oxo)-λ⁶-sulfaneylidene)carbamate (160 mg, 97.7%) as a yellow oil. LCMS (ESI) calcd. for C₃₃H₃₄FN₄O₇S [M+H]⁺ m/z 649.22, found 649.10.

Step 2: (R)-5-(2,3-dihydrobenzo[b][1,4]dioxin-6-yl)-2-((6-fluoro-2-methylpyridin-3-yl)oxy)-4-methyl-N-(3-(S-methylsulfonimidoyl)phenyl)nicotinamide: A solution of give tert-butyl (R)-((3-(5-(2,3-dihydrobenzo[b][1,4]dioxin-6-yl)-2-((6-fluoro-2-methylpyridin-3-yl)oxy)-4-methylnicotinamido)phenyl)(methyl)(oxo)-λ⁶-sulfaneylidene)carbamate (160 mg, 0.247 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 45% to 50% MeCN/H₂O containing 0.05% NH₃) to provide (R)-5-(2,3-dihydrobenzo[b][1,4]dioxin-6-yl)-2-((6-fluoro-2-methylpyridin-3-yl)oxy)-4-methyl-N-(3-(S-methylsulfonimidoyl)phenyl)nicotinamide (72.5 mg, 53.6%) as a white solid. ¹H NMR (400 MHz, DMSO-d₆) δ 11.14 (s, 1H), 8.46 (s, 1H), 7.98 (s, 1H), 7.89 (d, J=8.0 Hz, 1H), 7.86-7.77 (m, 1H), 7.68 (d, J=7.8 Hz, 1H), 7.60 (t, J=7.9 Hz, 1H), 7.09 (dd, J=8.7, 3.4 Hz, 1H), 6.96 (d, J=8.3 Hz, 1H), 6.87 (d, J=1.9 Hz, 1H), 6.82 (dd, J=8.3, 2.0 Hz, 1H), 4.29 (s, 4H), 4.24 (s, 1H), 3.07 (s, 3H), 2.29 (d, J=7.2 Hz, 6H). LCMS (ESI) calcd. for C₂₈H₂₆FN₄O₅S [M+H]⁺ m/z 549.16, found 549.10.

### Example 5C

### (R)-2-(4-cyano-2-methoxyphenoxy)-4-methyl-N-(3-(S-methylsulfonimidoyl)phenyl)-5-(trifluoromethyl)nicotinamide

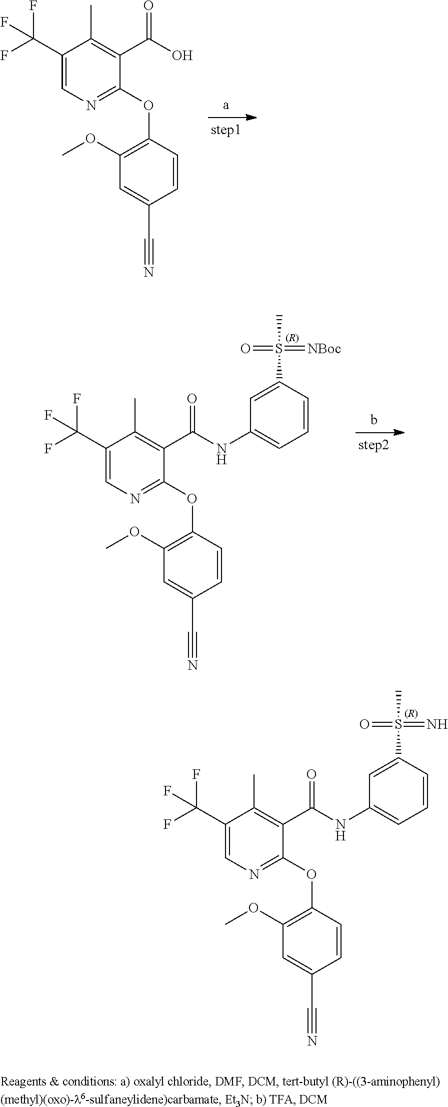

Step 1: tert-butyl (R)-((3-(2-(4-cyano-2-methoxyphenoxy)-4-methyl-5-(trifluoromethyl)nicotinamido)phenyl)(methyl)(oxo)-λ⁶-sulfaneylidene)carbamate: A solution of 2-(4-cyano-2-methoxyphenoxy)-4-methyl-5-(trifluoromethyl)nicotinic acid (150 mg, 0.43 mmol) in DCM (5 mL) was added DMF (4 drop) and oxalyl chloride (81 mg, 0.64 mmol) at room temperature. The reaction mixture was stirred at room temperature for 5 minutes to get crude solution. The crude solution was added to another solution of Et₃N (129 mg, 1.28 mmol) and tert-butyl (R)-((3-aminophenyl)(methyl)(oxo)-λ⁶-sulfaneylidene)carbamate (138 mg, 0.51 mmol) in DCM (5 mL) at 0° C. After the reaction was completed, the resulting solution was diluted with water (30 mL) and extracted with DCM (20 mL×3). The combined organic phases were washed with brine, dried over sodium sulfate, concentrated under vacuum. The residue was purified by flash column chromatography on silica gel (DCM/MeOH=10/1) to give tert-butyl (R)-((3-(2-(4-cyano-2-methoxyphenoxy)-4-methyl-5-(trifluoromethyl)nicotinamido)phenyl)(methyl)(oxo)-λ⁶-sulfaneylidene)carbamate (120 mg, 46.7%). LCMS (ESI) calcd. for C₂₈H₂₇F₃N₄O₆SNa [M+Na]⁺ m/z 627.15, found 627.10.

Step 2: (R)-2-(4-cyano-2-methoxyphenoxy)-4-methyl-N-(3-(S-methylsulfonimidoyl)phenyl)-5-(trifluoromethyl)nicotinamide: A solution of tert-butyl (R)-((3-(2-(4-cyano-2-methoxyphenoxy)-4-methyl-5-(trifluoromethyl)nicotinamido)phenyl)(methyl) (oxo)-λ⁶-sulfaneylidene)carbamate (120 mg, 0.19 mmol) in DCM (3 mL) was added TFA (0.3 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 30% to 95% MeCN/H₂O containing 0.1% formic acid) to provide (R)-2-(4-cyano-2-methoxyphenoxy)-4-methyl-N-(3-(S-methylsulfonimidoyl)phenyl)-5-(trifluoromethyl)nicotinamide (46.6 mg) as a white solid. ¹H NMR (400 MHz, DMSO-d₆) δ 11.14 (s, 1H), 8.52 (s, 1H), 8.41 (s, 1H), 7.89 (d, J=8.0 Hz, 1H), 7.69 (t, J=4.6 Hz, 2H), 7.61 (t, J=7.9 Hz, 1H), 7.52 (dd, J=8.2, 1.7 Hz, 1H), 7.41 (d, J=8.2 Hz, 1H), 4.24 (s, 1H), 3.77 (s, 3H), 3.07 (s, 3H), 2.48 (s, 3H). LCMS (ESI) calcd. for C₂₃H₂₀F₃N₄O₄S [M+H]⁺ m/z 505.12, found 505.00.

### Example 6C

### (R)-5-(3,4-difluorophenyl)-2-((6-fluoro-2-methylpyridin-3-yl)oxy)-4-methyl-N-(3-(S-methylsulfonimidoyl)phenyl)nicotinamide

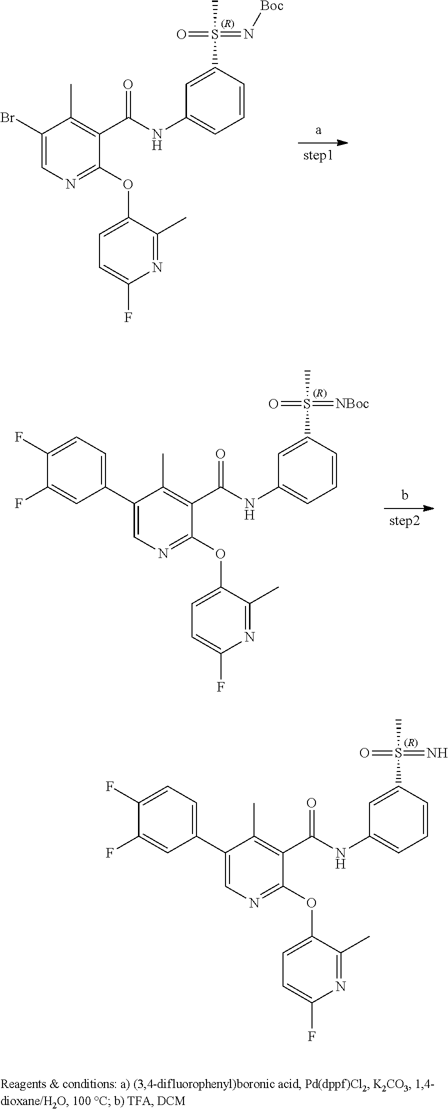

Step 1: tert-butyl (R)-((3-(5-(3,4-difluorophenyl)-2-((6-fluoro-2-methylpyridin-3-yl)oxy)-4-methylnicotinamido)phenyl)(methyl)(oxo)-λ⁶-sulfaneylidene)carbamate: A mixture of tert-butyl (R)-((3-(5-bromo-2-((6-fluoro-2-methylpyridin-3-yl)oxy)-4-methylnicotinamido)phenyl)(methyl)(oxo)-λ⁶-sulfaneylidene)carbamate (260 mg, 0.44 mmol), (3,4-difluorophenyl)boronic acid (138.1 mg, 0.87 mmol), K₂CO₃ (181.4 mg, 1.3 mmol) and Pd(dppf)Cl₂ (64 mg, 0.09 mmol) in 1,4-dioxane/H₂O (4/1, 15 mL) was heated at 100° C. for 5 hour under an atmosphere of N2. After the reaction was completed, the mixture was cooled to room temperature. The resulting solution was diluted with water (40 mL) and extracted with EtOAc (40 mL×3). The combine organic phases were washed with brine, dried over Na₂SO₄, concentrated under vacuum. The residue was purified by flash column chromatography on silica gel (PE/EtAOc=1/1) to give tert-butyl (R)-((3-(5-(3,4-difluorophenyl)-2-((6-fluoro-2-methylpyridin-3-yl)oxy)-4-methylnicotinamido)phenyl)(methyl)(oxo)-λ⁶-sulfaneylidene)carbamate (270 mg, 98.5%) as a yellow oil. LCMS (ESI) calcd. for C₃₁H₃₀F₃N₄O₅S [M+H]⁺ m/z 627.19, found 627.10.

Step 2: (R)-5-(3,4-difluorophenyl)-2-((6-fluoro-2-methylpyridin-3-yl)oxy)-4-methyl-N-(3-(S-methylsulfonimidoyl)phenyl)nicotinamide: A solution of give tert-butyl (R)-((3-(5-(3,4-difluorophenyl)-2-((6-fluoro-2-methylpyridin-3-yl)oxy)-4-methylnicotinamido)phenyl)(methyl) (oxo)-λ⁶-sulfaneylidene)carbamate (150 mg, 0.24 mmol) in DCM (5 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 40% to 90% MeCN/H₂O containing 0.05% NH₃) to provide (R)-5-(3,4-difluorophenyl)-2-((6-fluoro-2-methylpyridin-3-yl)oxy)-4-methyl-N-(3-(S-methylsulfonimidoyl)phenyl)nicotinamide (72.3 mg, 57.2%) as a white solid. ¹H NMR (400 MHz, DMSO-d₆) δ 11.18 (s, 1H), 8.47 (s, 1H), 8.06 (s, 1H), 7.98-7.78 (m, 2H), 7.77-7.51 (m, 4H), 7.26 (s, 1H), 7.10 (dd, J=8.5, 2.9 Hz, 1H), 4.25 (s, 1H), 3.07 (s, 3H), 2.37-2.22 (m, 6H). LCMS (ESI) calcd. for C₂₆H₂₂F₃N₄O₃S [M+H]⁺ m/z 527.14, found 527.05.

### Example 7C

### (R)-6-(2,3-dihydrobenzo[b][1,4]dioxin-6-yl)-3-((6-fluoro-2-methylpyridin-3-yl)oxy)-5-methyl-N-(3-(S-methylsulfonimidoyl)phenyl)pyridazine-4-carboxamide

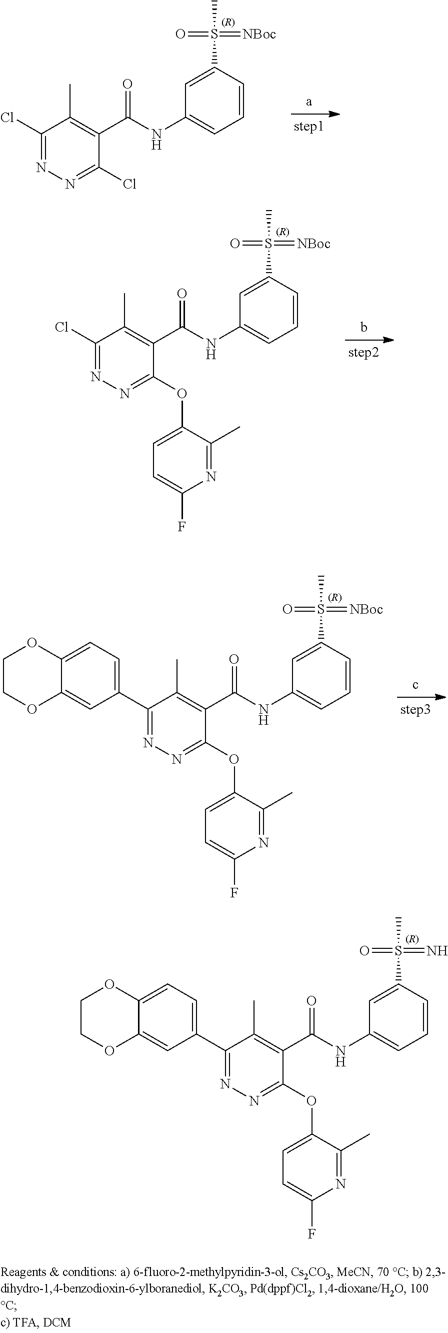

Step 1: tert-butyl (R)-((3-(6-chloro-3-((6-fluoro-2-methylpyridin-3-yl)oxy)-5-methylpyridazine-4-carboxamido)phenyl)(methyl)(oxo)-λ⁶-sulfaneylidene)carbamate: To a solution of tert-butyl (R)-((3-(3,6-dichloro-5-methylpyridazine-4-carboxamido)phenyl) (methyl)(oxo)-λ⁶-sulfaneylidene)carbamate (250 mg, 0.54 mmol) and 6-fluoro-2-methylpyridin-3-ol (104 mg, 0.82 mmol) in MeCN (15 mL) was added Cs₂CO₃ (621 mg, 1.91 mmol). The mixture was heated at 70° C. for 16 hours. After the reaction was completed, the mixture was concentrated under vacuum and directly purified by flash column chromatography on silica gel (PE/EtOAc=4/1 to 3/1) to provide tert-butyl (R)-((3-(6-chloro-3-((6-fluoro-2-methylpyridin-3-yl)oxy)-5-methylpyridazine-4-carboxamido)phenyl)(methyl)(oxo)-λ⁶-sulfaneylidene)carbamate (250 mg, 83.4%) as a yellow solid. LCMS (ESI) calcd. for C₂₄H₂₆C₁FN₅O₅S [M+H]⁺ m/z 550.13, found 550.15.

Step 2: tert-butyl (R)-((3-(6-(2,3-dihydrobenzo[b][1,4]dioxin-6-yl)-3-((6-fluoro-2-methylpyridin-3-yl)oxy)-5-methylpyridazine-4-carboxamido)phenyl)(methyl)(oxo)-λ⁶-sulfaneylidene)carbamate: A solution of tert-butyl (R)-((3-(6-chloro-3-((6-fluoro-2-methylpyridin-3-yl)oxy)-5-methylpyridazine-4-carboxamido)phenyl)(methyl)(oxo)-λ⁶-sulfaneylidene)carbamate (150 mg, 0.27 mmol), 2,3-dihydro-1,4-benzodioxin-6-ylboranediol (123 mg, 0.68 mmol), K₂CO₃ (132 mg, 0.95 mmol) and Pd(dppf)Cl₂ (20 mg, 0.03 mmol) in 1,4-dioxane (16 mL) and H₂O (4 mL) was heated at 100° C. for 16 hours. LCMS showed the reaction was completed. The mixture was diluted with water (80 mL) and extracted with EtOAc (30 mL×3). The combined organic layers were washed with brine, dried over sodium sulfate, concentrated under vacuum. The residue was purified by flash column chromatography on silica gel (PE/EtOAc=1/2) to provide tert-butyl (R)-((3-(6-(2,3-dihydrobenzo[b][1,4]dioxin-6-yl)-3-((6-fluoro-2-methylpyridin-3-yl)oxy)-5-methylpyridazine-4-carboxamido)phenyl)(methyl)(oxo)-λ⁶-sulfaneylidene)carbamate (160 mg, 90.24%) as a yellow solid. LCMS (ESI) calcd. for C₃₂H₃₃FN₅O₇S [M+H]⁺ m/z 650.21, found 650.20.

Step 3: (R)-6-(2,3-dihydrobenzo[b][1,4]dioxin-6-yl)-3-((6-fluoro-2-methylpyridin-3-yl)oxy)-5-methyl-N-(3-(S-methylsulfonimidoyl)phenyl)pyridazine-4-carboxamide: A solution of tert-butyl (R)-((3-(6-(2,3-dihydrobenzo[b][1,4]dioxin-6-yl)-3-((6-fluoro-2-methylpyridin-3-yl)oxy)-5-methylpyridazine-4-carboxamido)phenyl)(methyl)(oxo)-λ⁶-sulfaneylidene)carbamate (150 mg, 0.23 mmol) in DCM (3 mL) was added TFA (0.3 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 20% to 95% MeCN/H₂O containing 0.05% NH₃) to provide (R)-6-(2,3-dihydrobenzo[b][1,4]dioxin-6-yl)-3-((6-fluoro-2-methylpyridin-3-yl)oxy)-5-methyl-N-(3-(S-methylsulfonimidoyl)phenyl)pyridazine-4-carboxamide (59.8 mg, 45.5%) as a white solid. ¹H NMR (400 MHz, DMSO-d₆) δ 11.31 (s, 1H), 8.40 (s, 1H), 7.97-7.82 (m, 2H), 7.72 (d, J=7.8 Hz, 1H), 7.63 (t, J=7.9 Hz, 1H), 7.14 (dd, J=8.7, 3.4 Hz, 1H), 7.09 (d, J=1.8 Hz, 1H), 7.06-6.98 (m, 2H), 4.31 (s, 4H), 4.26 (s, 1H), 3.07 (s, 3H), 2.37 (s, 3H), 2.30 (s, 3H). LCMS (ESI) calcd. for C₂₇H₂₅FN₅O₅S [M+H]⁺ m/z 550.16, found 550.10.

### Example 8C

### 2-((6-fluoro-2-methylpyridin-3-yl)oxy)-4-methyl-N-(3-sulfamoylphenyl)-5-(trifluoromethyl)nicotinamide

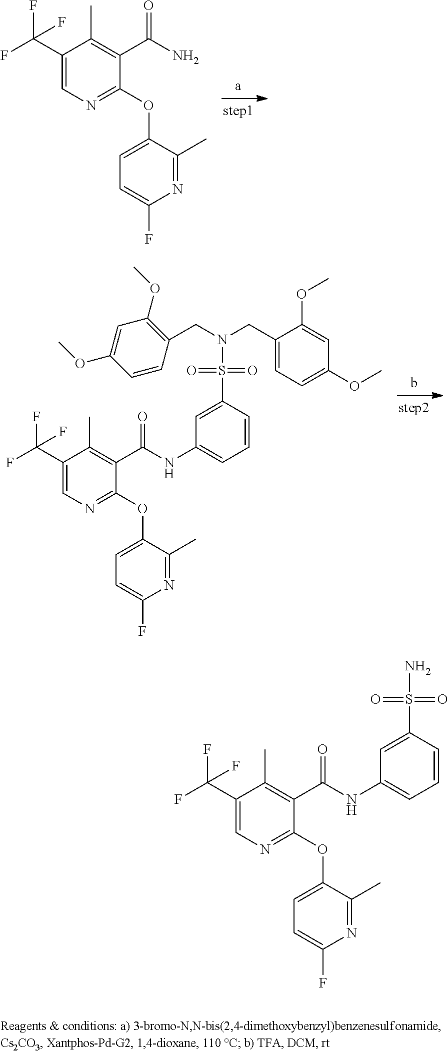

Step 1: N-(3-(N,N-bis(2,4-dimethoxybenzyl)sulfamoyl)phenyl)-2-((6-fluoro-2-methylpyridin-3-yl)oxy)-4-methyl-5-(trifluoromethyl)nicotinamide: A solution of 2-((6-fluoro-2-methylpyridin-3-yl)oxy)-4-methyl-5-(trifluoromethyl)nicotinamide (See: Example 11C; 100 mg, 0.30 mmol), 3-bromo-N,N-bis(2,4-dimethoxybenzyl)benzenesulfonamide (650 mg, 1.22 mmol), Cs₂CO₃ (494 mg, 1.52 mmol) and Xantphos-Pd-G2 (108 mg, 0.12 mmol) in dioxane (10 mL) was heated at 110° C. for 8 hours. LCMS showed the reaction was completed. The mixture was diluted with water (40 mL) and extracted with EtOAc (20 mL×3). The combined organic layers were washed with brine, dried over sodium sulfate, concentrated under vacuum. The residue was purified by flash column chromatography on silica gel (PE/EtOAc=3/1) to provide N-(3-(N,N-bis(2,4-dimethoxybenzyl)sulfamoyl)phenyl)-2-((6-fluoro-2-methylpyridin-3-yl)oxy)-4-methyl-5-(trifluoromethyl)nicotinamide (120 mg, 50%) as yellow solid. LCMS (ESI) calcd. for C₃₈H₃₆F₄N408SNa [M+Na]⁺ m/z 807.21, found 807.15.

Step 2: 2-((6-fluoro-2-methylpyridin-3-yl)oxy)-4-methyl-N-(3-sulfamoylphenyl)-5-(trifluoromethyl)nicotinamide A solution of N-(3-(N,N-bis(2,4-dimethoxybenzyl)sulfamoyl)phenyl)-2-((6-fluoro-2-methylpyridin-3-yl)oxy)-4-methyl-5-(trifluoromethyl)nicotinamide (110 mg, 0.14 mmol) in DCM (3 mL) was added TFA (0.3 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 40% to 95% MeCN/H₂O containing 0.05% NH₃H₂O) to provide 2-((6-fluoro-2-methylpyridin-3-yl)oxy)-4-methyl-N-(3-sulfamoylphenyl)-5-(trifluoromethyl)nicotinamide (32.6 mg, 47.1%) as a white solid. ¹H NMR (400 MHz, CD₃OD) δ 8.41 (s, 1H), 8.38 (t, J=1.9 Hz, 1H), 7.87-7.81 (m, 1H), 7.77-7.68 (m, 2H), 7.57 (t, J=8.0 Hz, 1H), 6.96 (dd, J=8.7, 3.2 Hz, 1H), 2.58 (d, J=1.1 Hz, 3H), 2.29 (s, 3H). LCMS (ESI) calcd. for C₂₀H₁₇F₄N₄O₄S [M+H]⁺ m/z 485.09, found 485.00.

### Example 9C

### (R)-3-((6-fluoro-2-methylpyridin-3-yl)oxy)-5-methyl-N-(3-(S-methylsulfonimidoyl)phenyl)-6-(trifluoromethyl)pyridazine-4-carboxamide

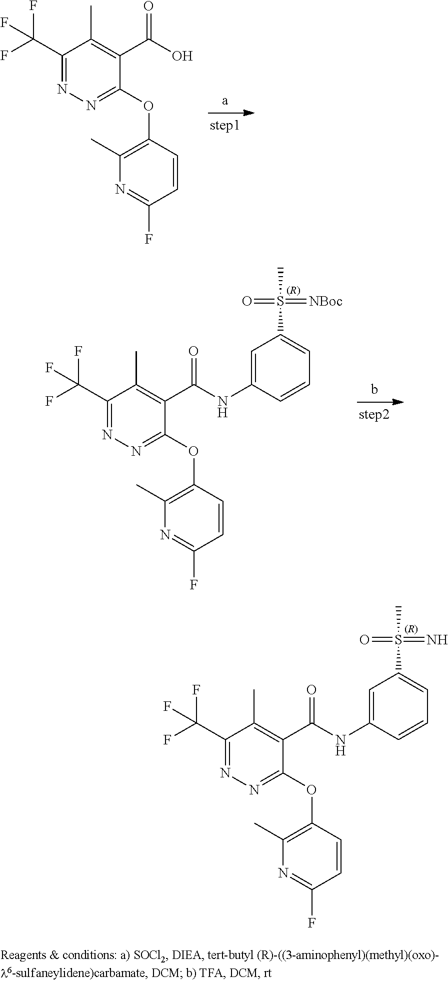

Step 1: tert-butyl (R)-((3-(3-((6-fluoro-2-methylpyridin-3-yl)oxy)-5-methyl-6-(trifluoromethyl)pyridazine-4-carboxamido)phenyl)(methyl)(oxo)-λ⁶-sulfaneylidene)carbamate: A solution of 3-((6-fluoro-2-methylpyridin-3-yl)oxy)-5-methyl-6-(trifluoromethyl)pyridazine-4-carboxylic acid (100 mg, 0.31 mmol) in SOCl₂ (2 mL) was heated to 50° C. and stirred for 1 hour. Then the solution was concentrated under vacuum to provide the chloride intermediate. The chloride intermediate was added to a solution of tert-butyl (R)-((3-aminophenyl)(methyl) (oxo)-λ⁶-sulfaneylidene)carbamate (82 mg, 0.31 mmol) and DIEA (80 mg, 0.62 mmol) in DCM (2 mL) at 0° C. The resulting mixture was stirred at 25° C. for 1 hour. Then the mixture was quenched with water (20 mL) and extracted with DCM (20 mL×2). The combined organic layers were washed with brine, dried over sodium sulfate, concentrated under vacuum. The residue was purified by flash column chromatography on silica gel (PE/EtOAc=1/1) to afford tert-butyl (R)-((3-(3-((6-fluoro-2-methylpyridin-3-yl)oxy)-5-methyl-6-(trifluoromethyl)pyridazine-4-carboxamido)phenyl)(methyl)(oxo)-λ⁶-sulfaneylidene)carbamate (100 mg, 55%) as a yellow solid. LCMS (ESI) calcd. for C₂₅H₂₆F₄N₅O₅S [M+H]⁺ m/z 584.16, found 584.10.

Step 2: (R)-3-((6-fluoro-2-methylpyridin-3-yl)oxy)-5-methyl-N-(3-(S-methylsulfonimidoyl)phenyl)-6-(trifluoromethyl)pyridazine-4-carboxamide: A solution of tert-butyl (R)-((3-(3-((6-fluoro-2-methylpyridin-3-yl)oxy)-5-methyl-6-(trifluoromethyl)pyridazine-4-carboxamido)phenyl)(methyl)(oxo)-λ⁶-sulfaneylidene)carbamate (100 mg, 0.17 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (30 mL×3). The combined organic phases were washed with brine, dried over sodium sulfate, concentrated under vacuum. The residue was purified by prep-HPLC (Gemini 5 um C₁₈ column, 150*21.2 mm, eluting with 40% to 90% MeCN/H₂O containing 0.05% NH₃) to provide (R)-3-((6-fluoro-2-methylpyridin-3-yl)oxy)-5-methyl-N-(3-(S-methylsulfonimidoyl)phenyl)-6-(trifluoromethyl)pyridazine-4-carboxamide (63.5 mg, 64.7%) as a white solid. ¹H NMR (400 MHz, CD₃OD) δ 8.45 (t, J=1.9 Hz, 1H), 7.96 (dd, J=8.1, 1.2 Hz, 1H), 7.91-7.82 (m, 2H), 7.68 (t, J=8.0 Hz, 1H), 7.03 (dd, J=8.7, 3.2 Hz, 1H), 3.19 (s, 3H), 2.63 (d, J=1.4 Hz, 3H), 2.36 (s, 3H). LCMS (ESI) calcd. for C₂₀H₁₈F₄N₅O₃S [M+H]⁺ m/z 484.10, found 484.00.

### Examples 10C and 11C

### (S)-2-((6-fluoro-2-methylpyridin-3-yl)oxy)-4-methyl-N-(2-(S-methylsulfonimidoyl)pyridin-4-yl)-5-(trifluoromethyl)nicotinamide and (R)-2-((6-fluoro-2-methylpyridin-3-yl)oxy)-4-methyl-N-(2-(S-methylsulfonimidoyl)pyridin-4-yl)-5-(trifluoromethyl)nicotinamide

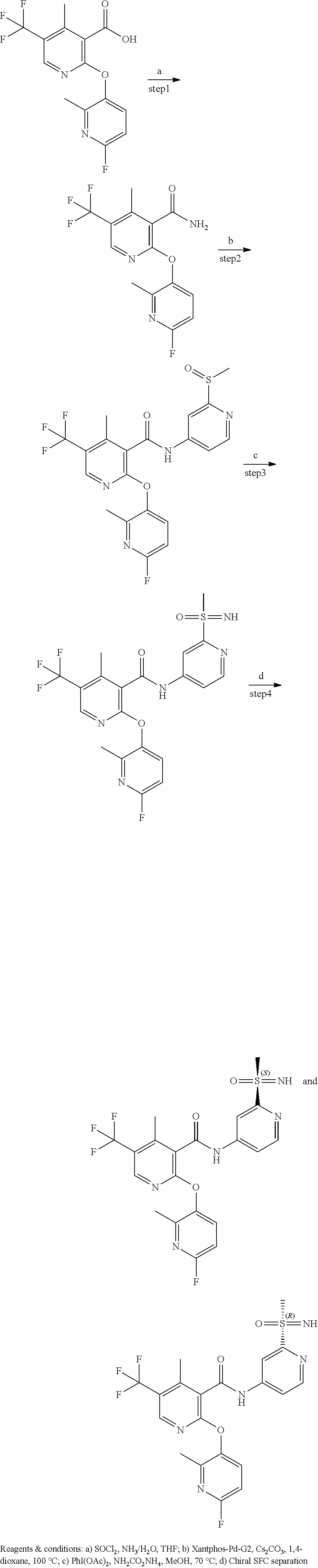

Step 1: 2-((6-fluoro-2-methylpyridin-3-yl)oxy)-4-methyl-5-(trifluoromethyl)nicotinamide: A solution of 3-(4,4-difluoroazepan-1-yl)-5-methyl-6-phenylpyridazine-4-carboxylic acid (500 mg, 1.515 mmol) in SOCl₂ (5 mL) was heated at 80° C. for 1 hour. After the reaction was completed, the reaction mixture was concentrated. The residue was diluted with THF and added dropwise to a stirred solution of ammonium hydroxide (5 mL). Then the mixture was stirred at room temperature for 1 hour. After the reaction was completed, the mixture was diluted with water (10 mL) and extracted with DCM (10 mL×3). The combined organic layers were washed with brine, dried over sodium sulfate, concentrated under vacuum. The residue was purified by flash column chromatography on silica gel (PE/EtOAc=1/1) to give 2-((6-fluoro-2-methylpyridin-3-yl)oxy)-4-methyl-5-(trifluoromethyl)nicotinamide (450 mg, 90.00%) as a white solid. LCMS (ESI) calcd. for C₁₄H₁₂F₄N₃O₂ [M+H]⁺ m/z 330.09, found 329.90.

Step 2: 2-((6-fluoro-2-methylpyridin-3-yl)oxy)-4-methyl-N-(2-(methylsulfinyl)pyridin-4-yl)-5-(trifluoromethyl)nicotinamide: A solution of 2-((6-fluoro-2-methylpyridin-3-yl)oxy)-4-methyl-5-(trifluoromethyl)nicotinamide (450 mg, 1.368 mmol) in 1,4-dioxane (8 mL) was added 4-bromo-2-(methylsulfinyl)pyridine (449.39 mg, 2.052 mmol), Cs₂CO₃ (1337 mg, 4.104 mmol) and Xantphos-Pd-G2 (122 mg, 0.137 mmol) at room temperature. The reaction mixture was stirred at 100° C. for 16 hours under nitrogen. The reaction was monitored by LCMS. After the reaction was completed, the filtrate was diluted with water (20 mL) and extracted with DCM (20 mL×3). The combined organic layers were washed with brine, dried over sodium sulfate, concentrated under vacuum. The residue was purified by flash column chromatography on silica gel (PE/EtOAc=1/1) to give 2-((6-fluoro-2-methylpyridin-3-yl)oxy)-4-methyl-N-(2-(methylsulfinyl)pyridin-4-yl)-5-(trifluoromethyl)nicotinamide (290 mg, 21.50%) as a white solid. LCMS (ESI) calcd. for C₂₀H₁₇F₄N₄O₃S [M+H]⁺ m/z 469.10, found 469.00.

Step 3: 2-((6-fluoro-2-methylpyridin-3-yl)oxy)-4-methyl-N-(2-(S-methylsulfonimidoyl)pyridin-4-yl)-5-(trifluoromethyl)nicotinamide: To a solution of 2-((6-fluoro-2-methylpyridin-3-yl)oxy)-4-methyl-N-(2-(methylsulfinyl)pyridin-4-yl)-5-(trifluoromethyl)nicotinamide (290 mg, 0.618 mmol) in MeOH (5 mL) was added PhI(OAc)₂ (498 mg, 1.545 mmol) and ammonium carbamate (145 mg, 1.854 mmol) at room temperature. The reaction mixture was heated at 70° C. for 1 hour. After the reaction was completed, the mixture was cooled to room temperature, diluted with water (5 mL), and extracted with DCM (5 mL×3). The combined organic layers were washed with brine, dried over sodium sulfate, concentrated under vacuum. The residue was first purified by flash column chromatography on silica gel (PE/EtOAc=1/1), then further purified by prep-HPLC (Gemini 5 um C₁₈ column, 150*21.2 mm, eluting with 30% to 90% MeCN/H₂O containing 0.1% formic acid) to afford 2-((6-fluoro-2-methylpyridin-3-yl)oxy)-4-methyl-N-(2-(S-methylsulfonimidoyl)pyridin-4-yl)-5-(trifluoromethyl)nicotinamide (62.4 mg, 21.53%) as a white solid. ¹H NMR (400 MHz, DMSO-d₆) δ 11.64 (s, 1H), 8.65 (d, J=5.2 Hz, 1H), 8.57 (s, 1H), 8.46 (s, 1H), 7.86-7.79 (m, 2H), 7.13-7.10 (m, 1H), 4.40 (s, 1H), 3.15 (s, 3H), 2.49 (s, 3H), 2.24 (s, 3H). LCMS (ESI) calcd. for C₂₀H₁₈F₄N₅O₃S [M+H]⁺ m/z 484.11, found 484.00.

Step 4: (S)-2-((6-fluoro-2-methylpyridin-3-yl)oxy)-4-methyl-N-(2-(S-methylsulfonimidoyl)pyridin-4-yl)-5-(trifluoromethyl)nicotinamide and (R)-2-((6-fluoro-2-methylpyridin-3-yl)oxy)-4-methyl-N-(2-(S-methylsulfonimidoyl)pyridin-4-yl)-5-(trifluoromethyl)nicotinamide: 2-((6-fluoro-2-methylpyridin-3-yl)oxy)-4-methyl-N-(2-(S-)pyridin-4-yl)-5-(trifluoromethyl)nicotinamide (80 mg) was purified by Chiral-Prep-HPLC (Chiralpak-IC, 4.6*250 mm, eluting with 20% CO₂-MeOH containing 0.1% NH₃) to afford (S)-2-((6-fluoro-2-methylpyridin-3-yl)oxy)-4-methyl-N-(2-(S-methylsulfonimidoyl)pyridin-4-yl)-5-(trifluoromethyl)nicotinamide (37 mg) as the first eluting isomer ¹H NMR (400 MHz, DMSO-d₆, ppm) δ 11.64 (s, 1H), 8.65 (d, J=5.2 Hz, 1H), 8.57 (s, 1H), 8.46 (m, 1H), 7.86-7.79 (m, 2H), 7.13-7.10 (m, 1H), 4.41 (s, 1H), 3.15 (s, 3H), 2.50 (s, 3H), 2.24 (s, 3H). LCMS (ESI) calcd. for C₂₀H₁₈F₄N₅O₃S [M+H]⁺ m/z 484.11, found 484.05 and the second eluting isomer (R)-2-((6-fluoro-2-methylpyridin-3-yl)oxy)-4-methyl-N-(2-(S-methylsulfonimidoyl)pyridin-4-yl)-5-(trifluoromethyl)nicotinamide (35 mg) as the second elution. ¹H NMR (400 MHz, DMSO-d₆) δ 11.64 (s, 1H), 8.65 (d, J=5.2 Hz, 1H), 8.57 (s, 1H), 8.46 (m, 1H), 7.86-7.79 (m, 2H), 7.13-7.10 (m, 1H), 4.41 (s, 1H), 3.15 (s, 3H), 2.50 (s, 3H), 2.24 (s, 3H). LCMS (ESI) calcd. for C₂₀H₁₈F₄N₅O₃S [M+H]⁺ m/z 484.11, found 484.05.

### Example 12C

### (R)-5-cyclopropyl-2-((6-fluoro-2-methylpyridin-3-yl)oxy)-4-methyl-N-(3-(S-methylsulfonimidoyl)phenyl)nicotinamide

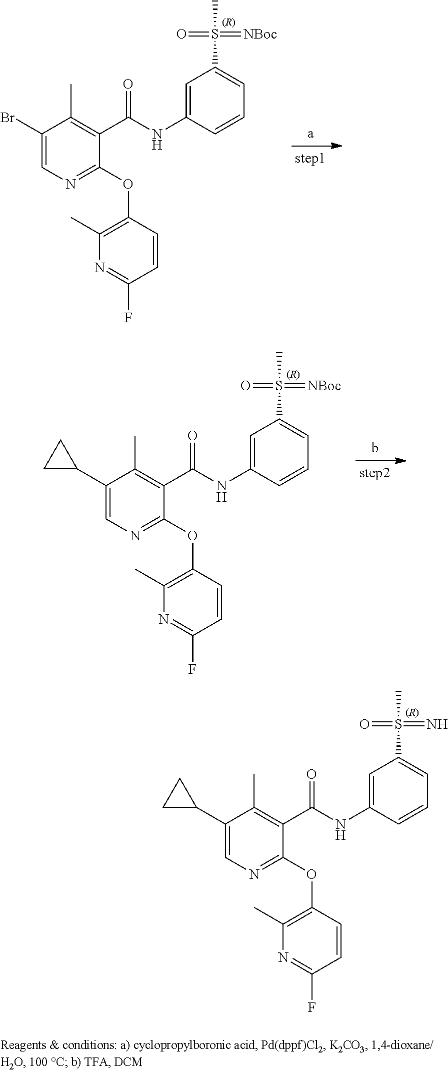

Step 1: tert-butyl (R)-((3-(5-cyclopropyl-2-((6-fluoro-2-methylpyridin-3-yl)oxy)-4-methylnicotinamido)phenyl)(methyl)(oxo)-λ⁶-sulfaneylidene)carbamate: A solution of tert-butyl (R)-((3-(5-bromo-2-((6-fluoro-2-methylpyridin-3-yl)oxy)-4-methylnicotinamido)phenyl) (methyl)(oxo)-λ⁶-sulfaneylidene)carbamate (100 mg, 0.17 mmol), cyclopropylboronic acid (116 mg, 1.35 mmol), K₂CO₃ (93 mg, 0.67 mmol) and Pd(dppf)Cl₂ (12 mg, 0.02 mmol) in dioxane (10 mL) and H₂O (2.5 mL) was heated at 100° C. for 8 hours under N2 atmosphere. LCMS showed the reaction was completed. The mixture was diluted with water (10 mL) and extracted with EtOAc (10 mL×3). The combined organic layers were washed with brine, dried over sodium sulfate, concentrated under vacuum. The residue was purified by flash column chromatography on silica gel (PE/EtOAc=1/10) to provide tert-butyl (R)-((3-(5-cyclopropyl-2-((6-fluoro-2-methylpyridin-3-yl)oxy)-4-methylnicotinamido)phenyl)(methyl)(oxo)-λ⁶-sulfaneylidene)carbamate (90 mg, 96.17%) as yellow oil. LCMS (ESI) calcd. for C₂₈H₃₂FN₄O₅S [M+H]⁺ m/z 555.21, found 555.20.

Step 2: (R)-5-cyclopropyl-2-((6-fluoro-2-methylpyridin-3-yl)oxy)-4-methyl-N-(3-(S-methylsulfonimidoyl)phenyl)nicotinamide: A solution of tert-butyl (R)-((3-(5-cyclopropyl-2-((6-fluoro-2-methylpyridin-3-yl)oxy)-4-methylnicotinamido)phenyl)(methyl)(oxo)-λ⁶-sulfaneylidene)carbamate (90 mg, 0.16 mmol) in DCM (3 mL) was added TFA (0.3 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 40% to 95% MeCN/H₂O containing 0.05% NH₃H₂O) to provide (R)-5-cyclopropyl-2-((6-fluoro-2-methylpyridin-3-yl)oxy)-4-methyl-N-(3-(S-methylsulfonimidoyl)phenyl)nicotinamide (38.95 mg, 52.80%) as a white solid. ¹H NMR (400 MHz, DMSO-d₆) δ 11.04 (s, 1H), 8.45 (t, J=1.8 Hz, 1H), 7.91-7.82 (m, 2H), 7.78-7.70 (m, 1H), 7.67 (d, J=7.9 Hz, 1H), 7.59 (t, J=7.9 Hz, 1H), 7.05 (dd, J=8.6, 3.5 Hz, 1H), 4.23 (s, 1H), 3.06 (s, 3H), 2.44 (s, 3H), 2.22 (s, 3H), 1.92-1.82 (m, 1H), 0.97-0.90 (m, 2H), 0.68-0.61 (m, 2H). LCMS (ESI) calcd. for C₂₃H₂₄FN₄O₃S [M+H]⁺ m/z 455.16, found 455.10.

### Example 13C

### 3-((6-fluoro-2-methylpyridin-3-yl)oxy)-5-methyl-N-(3-sulfamoylphenyl)-6-(trifluoromethyl)pyridazine-4-carboxamide

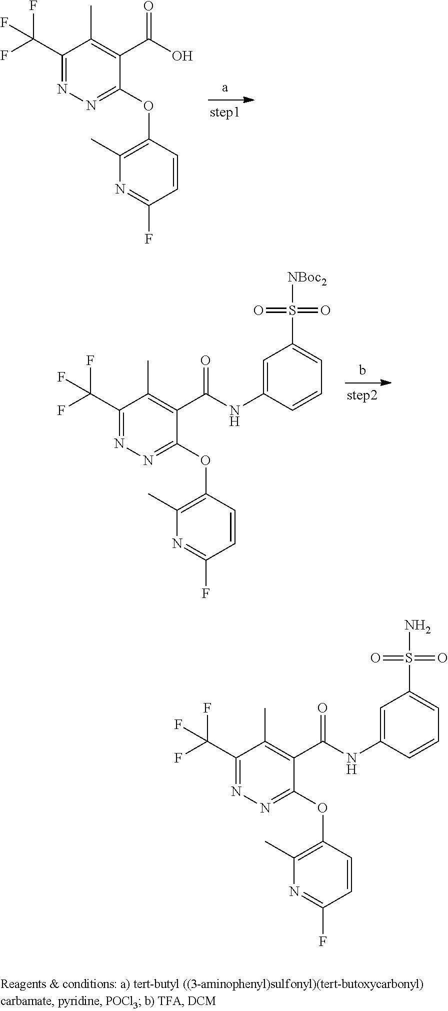

Step 1: tert-butyl (tert-butoxycarbonyl)((3-(3-((6-fluoro-2-methylpyridin-3-yl)oxy)-5-methyl-6-(trifluoromethyl)pyridazine-4-carboxamido)phenyl)sulfonyl)carbamate: A mixture of 3-((6-fluoro-2-methylpyridin-3-yl)oxy)-5-methyl-6-(trifluoromethyl)pyridazine-4-carboxylic acid (100 mg, 0.30 mmol), tert-butyl ((3-aminophenyl)sulfonyl)(tert-butoxycarbonyl)carbamate (82 mg, 0.35 mmol) in pyridine (5 mL) was added POCl₃ (150 μL) dropwise at room temperature. The reaction solution was stirred at room temperature for 1 hour. Then the mixture was quenched with water (20 mL) and extracted with DCM (20 mL×2). The combined organic layers were washed with brine, dried over sodium sulfate, concentrated under vacuum. The residue was purified by flash column chromatography on silica gel (PE/EtOAc=1/1) to afford tert-butyl (tert-butoxycarbonyl)((3-(3-((6-fluoro-2-methylpyridin-3-yl)oxy)-5-methyl-6-(trifluoromethyl)pyridazine-4-carboxamido)phenyl)sulfonyl)carbamate (80 mg, 38.8%) as a yellow solid. LCMS (ESI) calcd. for C₂₄H₂₄F₄N₅O₆S [M−Boc+H]⁺ m/z 586.19, found 586.10.

Step 2: 3-((6-fluoro-2-methylpyridin-3-yl)oxy)-5-methyl-N-(3-sulfamoylphenyl)-6-(trifluoromethyl)pyridazine-4-carboxamide: A solution of tert-butyl (tert-butoxycarbonyl)((3-(3-((6-fluoro-2-methylpyridin-3-yl)oxy)-5-methyl-6-(trifluoromethyl)pyridazine-4-carboxamido)phenyl)sulfonyl)carbamate (80 mg, 0.12 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (30 mL×3). The combined organic layers were washed with brine, dried over sodium sulfate, concentrated under vacuum. The residue was purified by prep-HPLC (Gemini 5 um C₁₈ column, 150*21.2 mm, eluting with 40% to 90% MeCN/H₂O containing 0.05% NH₃) to provide 3-((6-fluoro-2-methylpyridin-3-yl)oxy)-5-methyl-N-(3-sulfamoylphenyl)-6-(trifluoromethyl)pyridazine-4-carboxamide (30.50 mg, 52.2%) as a white solid. ¹H NMR (400 MHz, DMSO-d₆) δ 11.32 (s, 1H), 8.32 (s, 1H), 8.00-7.88 (m, 1H), 7.77 (d, J=7.3 Hz, 1H), 7.66-7.57 (m, 2H), 7.45 (s, 2H), 7.17 (dd, J=8.9, 3.3 Hz, 1H), 2.53 (s, 3H), 2.29 (s, 3H). LCMS (ESI) calcd. for C₁₉H₁₆F₄N₅O₄S [M+H]⁺ m/z 486.09, found 486.00.

### Examples 14C and 15C

### (S)-2-((6-fluoro-2-methylpyridin-3-yl)oxy)-N-(4-fluoro-3-(S-methylsulfonimidoyl)phenyl)-4-methyl-5-(trifluoromethyl)nicotinamide and (R)-2-((6-fluoro-2-methylpyridin-3-yl)oxy)-N-(4-fluoro-3-(S-methylsulfonimidoyl)phenyl)-4-methyl-5-(trifluoromethyl)nicotinamide

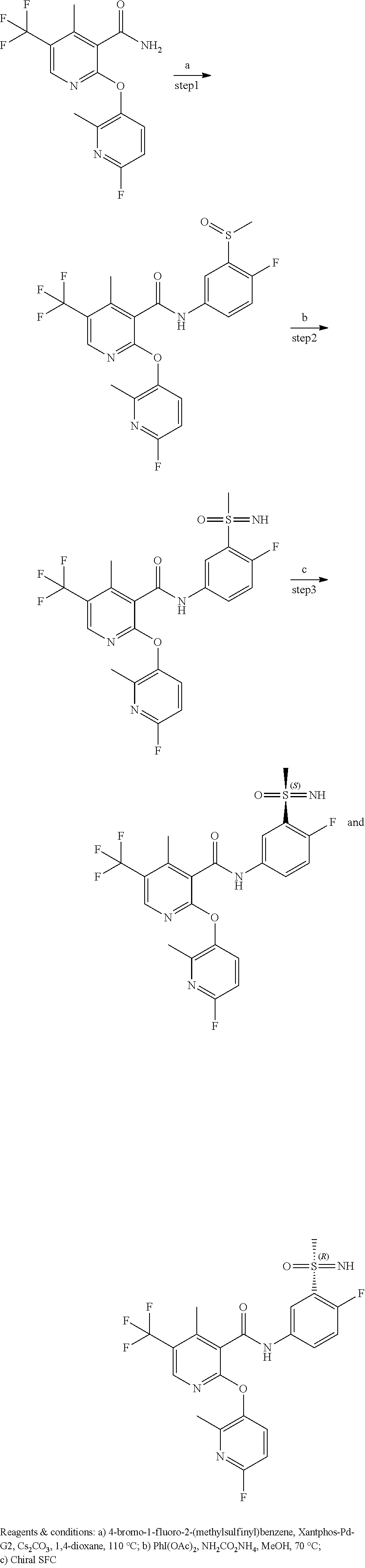

Step 1: 2-((6-fluoro-2-methylpyridin-3-yl)oxy)-N-(4-fluoro-3-(methylsulfinyl)phenyl)-4-methyl-5-(trifluoromethyl)nicotinamide: A solution of 2-((6-fluoro-2-methylpyridin-3-yl)oxy)-4-methyl-5-(trifluoromethyl)nicotinamide (See Example 8C, 400 mg, 1.212 mmol) in dioxane (8 mL) was added 4-bromo-1-fluoro-2-(methylsulfinyl)benzene (572 mg, 2.424 mmol), cesium carbonate (1027 mg, 3.151 mmol) and Xantphos-Pd-G2 (108 mg, 0.121 mmol) at room temperature. The reaction mixture was stirred at 100° C. for 16 hours under nitrogen. The reaction was monitored by LCMS. After the reaction was completed, the filtrate was diluted with water (20 mL) and extracted with DCM (20 mL×3). The combined organic layers were washed with brine, dried over sodium sulfate, concentrated under vacuum. The residue was purified by flash column chromatography on silica gel (PE/EtOAc=1/1) to obtain 2-((6-fluoro-2-methylpyridin-3-yl)oxy)-N-(4-fluoro-3-(methylsulfinyl)phenyl)-4-methyl-5-(trifluoromethyl)nicotinamide (340 mg, 21.50%) as a white oil. LCMS (ESI) calcd. for C₂₁H₁₇F₅N₃O₃S [M+H]⁺ m/z 486.09, found 486.00.

Step 2: 2-((6-fluoro-2-methylpyridin-3-yl)oxy)-N-(4-fluoro-3-(S-methylsulfonimidoyl)phenyl)-4-methyl-5-(trifluoromethyl)nicotinamide: To a solution of 2-((6-fluoro-2-methylpyridin-3-yl)oxy)-N-(4-fluoro-3-(methylsulfinyl)phenyl)-4-methyl-5-(trifluoromethyl)nicotinamide (340 mg, 0.700 mmol) in MeOH (10 mL) was added PhI(OAc)₂ (563.5 mg, 1.75 mmol) and ammonium carbamate (164 mg, 2.1 mmol) at room temperature. The reaction mixture was heated at 70° C. for 1 hour. After the reaction was completed, the mixture was cooled to room temperature, diluted with water (5 mL), and extracted with DCM (5 mL×3). The combine organic phases were washed with brine, dried over sodium sulfate, concentrated under vacuum. The residue was first purified by flash column chromatography on silica gel (PE/EtOAc=1/1), then further purified by prep-HPLC (Gemini 5 um C₁₈ column, 150*21.2 mm, eluting with 30% to 90% MeCN/H₂O containing 0.1% formic acid) to afford 2-((6-fluoro-2-methylpyridin-3-yl)oxy)-N-(4-fluoro-3-(S-methylsulfonimidoyl)phenyl)-4-methyl-5-(trifluoromethyl)nicotinamide (180 mg, 51.42%) as a white solid. LCMS (ESI) calcd. for C₂₁H₁₈F₅N₄O₃S [M+H]⁺ m/z 501.10, found 501.05.

Step 3: (S)-2-((6-fluoro-2-methylpyridin-3-yl)oxy)-N-(4-fluoro-3-(S-methylsulfonimidoyl)phenyl)-4-methyl-5-(trifluoromethyl)nicotinamide and (R)-2-((6-fluoro-2-methylpyridin-3-yl)oxy)-N-(4-fluoro-3-(S-methylsulfonimidoyl)phenyl)-4-methyl-5-(trifluoromethyl)nicotinamide 2-((6-fluoro-2-methylpyridin-3-yl)oxy)-N-(4-fluoro-3-(S-methylsulfonimidoyl)phenyl)-4-methyl-5-(trifluoromethyl)nicotinamide was purified by Chiral-Prep-HPLC (Chiralpak-AD-H, 4.6*250 mm, eluting with 20% CO₂-MeOH containing 0.1% NH₃). First eluting compound (S)-2-((6-fluoro-2-methylpyridin-3-yl)oxy)-N-(4-fluoro-3-(S-methylsulfonimidoyl)phenyl)-4-methyl-5-(trifluoromethyl)nicotinamide as a white solid. 1H NMR (400 MHz, DMSO-d₆) δ 11.20 (s, 1H), 8.55 (s, 1H), 8.34 (dd, J=6.5, 2.8 Hz, 1H), 7.94 (dd, J=8.9, 4.2, 2.8 Hz, 1H), 7.84 (dd, J=8.7, 6.9 Hz, 1H), 7.51-7.43 (m, 1H), 7.12 (dd, J=8.7, 3.4 Hz, 1H), 4.73 (s, 1H), 3.20 (s, 3H), 2.49 (s, 3H), 2.24 (s, 3H). LCMS (ESI) calcd. for C₂₁H₁₈F₅N₄O₃S [M+H]⁺ m/z 501.10, found 501.05 and the second eluting compound (R)-2-((6-fluoro-2-methylpyridin-3-yl)oxy)-N-(4-fluoro-3-(S-methylsulfonimidoyl)phenyl)-4-methyl-5-(trifluoromethyl)nicotinamide as a white solid. ¹H NMR (400 MHz, DMSO-d₆) δ 11.20 (s, 1H), 8.55 (s, 1H), 8.34 (dd, J=6.5, 2.8 Hz, 1H), 7.94 (dd, J=8.9, 4.2, 2.8 Hz, 1H), 7.84 (dd, J=8.7, 6.9 Hz, 1H), 7.51-7.43 (m, 1H), 7.12 (dd, J=8.7, 3.4 Hz, 1H), 4.73 (s, 1H), 3.20 (s, 3H), 2.49 (s, 3H), 2.24 (s, 3H). LCMS (ESI) calcd. for C₂₁H₁₈F₅N₄O₃S [M+H]⁺ m/z 501.10, found 501.10.

### Examples 16C and 17C

### (S)-2-(4-cyano-2-methoxyphenoxy)-5-(4-(difluoromethyl)phenyl)-4-methyl-N-(2-(S-methylsulfonimidoyl)pyridin-4-yl)nicotinamide and (R)-2-(4-cyano-2-methoxyphenoxy)-5-(4-(difluoromethyl)phenyl)-4-methyl-N-(2-(S-methylsulfonimidoyl)pyridin-4-yl)nicotinamide

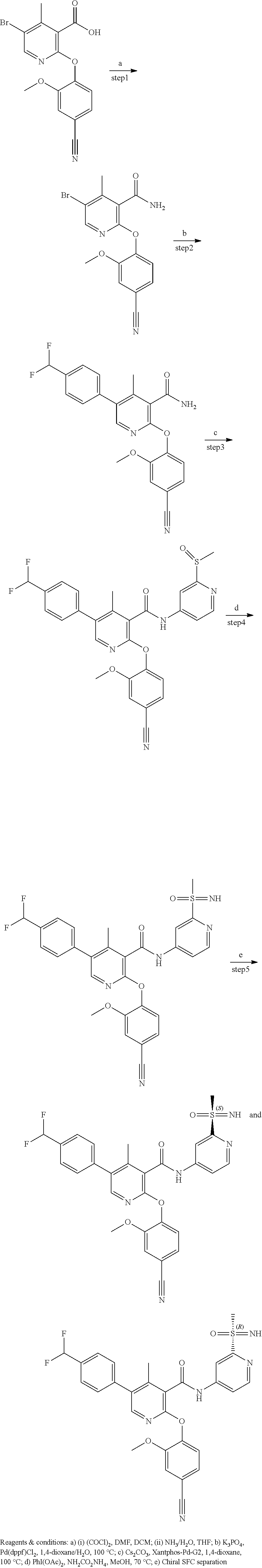

Step 1: 5-bromo-2-(4-cyano-2-methoxyphenoxy)-4-methylnicotinamide: A solution of 5-bromo-2-(4-cyano-2-methoxyphenoxy)-4-methylnicotinic acid (See: Example 20C; 300 mg, 0.83 mmol) in DCM (5 mL) was added oxalyl chloride (0.3 mL) and DMF (1 drop) at room temperature. After addition, the reaction mixture was stirred at room temperature for 1 hour. After the reaction was completed, the reaction mixture was concentrated. The residue was diluted with THF and added dropwise to a stirred solution of NH₃—H₂O (5 mL). Then the mixture was stirred at room temperature for 1 hour. After the reaction was completed, the mixture was diluted with water (10 mL) and extracted with DCM (10 mL×3). The combine organic phases were washed with brine, dried over sodium sulfate, concentrated under vacuum. The residue was purified by flash column chromatography on silica gel (PE/EtOAc=1/1) to give 5-bromo-2-(4-cyano-2-methoxyphenoxy)-4-methylnicotinamide (280 mg, 93.33%) as a white solid. LCMS (ESI) calcd. for C₁₅H₁₃BrN₃O₃ [M+H]⁺ m/z 362.02, found 361.85.

Step 2: 2-(4-cyano-2-methoxyphenoxy)-5-(4-(difluoromethyl)phenyl)-4-methylnicotinamide: A mixture 5-bromo-2-(4-cyano-2-methoxyphenoxy)-4-methylnicotinamide (280 mg, 0.77 mmol) and (4-(difluoromethyl)phenyl)boronic acid (200 mg, 1.16 mmol) in 1,4-dioxane/H₂O (10/1, 20 mL), K₃PO₄ (492 mg, 2.32 mmol) and [1,1′-Bis(diphenylphosphino)ferrocene]dichloropalladium (II) (57 mg, 0.08 mmol) was heated at 100° C. in a microwave reactor for 1 hours under an atmosphere of N2. The reaction was monitored by LCMS. After the reaction was completed, the mixture was cooled to room temperature. The resulting solution was diluted with water (20 mL) and extracted with DCM (20 mL×3). The combine organic phases were washed with brine, dried over Na₂SO₄, concentrated under vacuum. The residue was purified by flash column chromatography on silica gel (PE/EtAOc=3/1) to give 2-(4-cyano-2-methoxyphenoxy)-5-(4-(difluoromethyl)phenyl)-4-methylnicotinamide (300 mg, 92.93%) as a white oil. LCMS (ESI) calcd. for C₂₂H₁₈F₂N₃O₃ [M+H]⁺ m/z 410.13, found 410.00.

Step 3: 2-(4-cyano-2-methoxyphenoxy)-5-(4-(difluoromethyl)phenyl)-4-methyl-N-(2-(methylsulfinyl)pyridin-4-yl)nicotinamide: A solution of 2-(4-cyano-2-methoxyphenoxy)-5-(4-(difluoromethyl)phenyl)-4-methylnicotinamide (300 mg, 0.73 mmol) in dioxane (8 mL) was added 4-bromo-2-(methylsulfinyl)pyridine (461 mg, 2.19 mmol), cesium carbonate (620 mg, 1.90 mmol) and Xantphos-Pd-G2 (130 mg, 0.15 mmol) at room temperature. The reaction mixture was stirred at 100° C. for 16 hours under nitrogen. The reaction was monitored by LCMS. After the reaction was completed, the filtrate was diluted with water (20 mL) and extracted with DCM (20 mL×3). The combine organic phases were washed with brine, dried over sodium sulfate, concentrated under vacuum. The residue was purified by flash column chromatography on silica gel (PE/EtOAc=1/1) to give 2-(4-cyano-2-methoxyphenoxy)-5-(4-(difluoromethyl)phenyl)-4-methyl-N-(2-(methylsulfinyl)pyridin-4-yl)nicotinamide (210 mg, 52.36%) as a white solid. LCMS (ESI) calcd. for C₂₈H₂₃F₂N₄O₄S [M+H]⁺ m/z 549.14, found 549.05.

Step 4: 2-(4-cyano-2-methoxyphenoxy)-5-(4-(difluoromethyl)phenyl)-4-methyl-N-(2-(S-methylsulfonimidoyl)pyridin-4-yl)nicotinamide: To a solution of 2-(4-cyano-2-methoxyphenoxy)-5-(4-(difluoromethyl)phenyl)-4-methyl-N-(2-(methylsulfinyl)pyridin-4-yl)nicotinamide (210 mg, 0.38 mmol) in MeOH (5 mL) was added PhI(OAc)₂ (308 mg, 0.96 mmol) and ammonium carbamate (90 mg, 1.15 mmol) at room temperature. The reaction mixture was heated at 70° C. for 1 hour. After the reaction was completed, the mixture was cooled to room temperature, diluted with water (5 mL), and extracted with DCM (15 mL×3). The combined organic layers were washed with brine, dried over sodium sulfate, concentrated under vacuum. The residue was purified by prep-HPLC (Gemini 5 um C₁₈ column, 150*21.2 mm, eluting with 30% to 90% MeCN/H₂O containing 0.1% formic acid) to afford 2-(4-cyano-2-methoxyphenoxy)-5-(4-(difluoromethyl)phenyl)-4-methyl-N-(2-(S-methylsulfonimidoyl)pyridin-4-yl)nicotinamide (50 mg, 23.14%) as a white solid. LCMS (ESI) calcd. for C₂₈H₂₄F₂N₅O₄S [M+H]⁺ m/z 564.15, found 564.10.

Step 5: (S)-2-(4-cyano-2-methoxyphenoxy)-5-(4-(difluoromethyl)phenyl)-4-methyl-N-(2-(S-methylsulfonimidoyl)pyridin-4-yl)nicotinamide and (R)-2-(4-cyano-2-methoxyphenoxy)-5-(4-(difluoromethyl)phenyl)-4-methyl-N-(2-(S-methylsulfonimidoyl)pyridin-4-yl)nicotinamide 2-(4-cyano-2-methoxyphenoxy)-5-(4-(difluoromethyl)phenyl)-4-methyl-N-(2-(S-methylsulfonimidoyl)pyridin-4-yl)nicotinamide was purified by Chiral-Prep-HPLC (Chiralpak-IC, 4.6*250 mm, eluting with 20% CO₂-MeOH containing 0.1% NH₃). The first elution was lyophilized to afford (S)-2-(4-cyano-2-methoxyphenoxy)-5-(4-(difluoromethyl)phenyl)-4-methyl-N-(2-(S-methylsulfonimidoyl)pyridin-4-yl)nicotinamide (15.7 mg) as a white solid. ¹H NMR (400 MHz, DMSO-d₆) δ 11.64 (s, 1H), 8.59 (d, J=5.2 Hz, 1H), 8.02 (s, 1H), 7.66-7.57 (m, 4H), 7.43 (d, J=6.8 Hz, 1H), 7.37-7.33 (m, 3H), 7.17 (s, 1H), 7.14-6.87 (m, 1H), 4.17 (s, 1H), 3.77 (s, 3H), 3.12 (s, 3H), 2.07 (s, 3H). LCMS (ESI) calcd. for C₂₈H₂₄F₂N₅O₄S [M+H]⁺ m/z 564.15, found 564.15. The second elution was lyophilized to afford (R)-2-(4-cyano-2-methoxyphenoxy)-5-(4-(difluoromethyl)phenyl)-4-methyl-N-(2-(S-methylsulfonimidoyl)pyridin-4-yl)nicotinamide (12.8 mg) as a white solid. ¹H NMR (400 MHz, DMSO-d₆) δ 11.64 (s, 1H), 8.59 (d, J=5.2 Hz, 1H), 8.02 (s, 1H), 7.66-7.57 (m, 4H), 7.43 (d, J=6.8 Hz, 1H), 7.37-7.33 (m, 3H), 7.17 (s, 1H), 7.14-6.87 (m, 1H), 4.17 (s, 1H), 3.77 (s, 3H), 3.12 (s, 3H), 2.07 (s, 3H). LCMS (ESI) calcd. for C₂₈H₂₄F₂N₅O₄S [M+H]⁺ m/z 564.15, found 564.10.

### Example 20C

### (R)-2-(4-cyano-2-methoxyphenoxy)-5-(4-cyanophenyl)-4-methyl-N-(3-(S-methylsulfonimidoyl)phenyl)nicotinamide

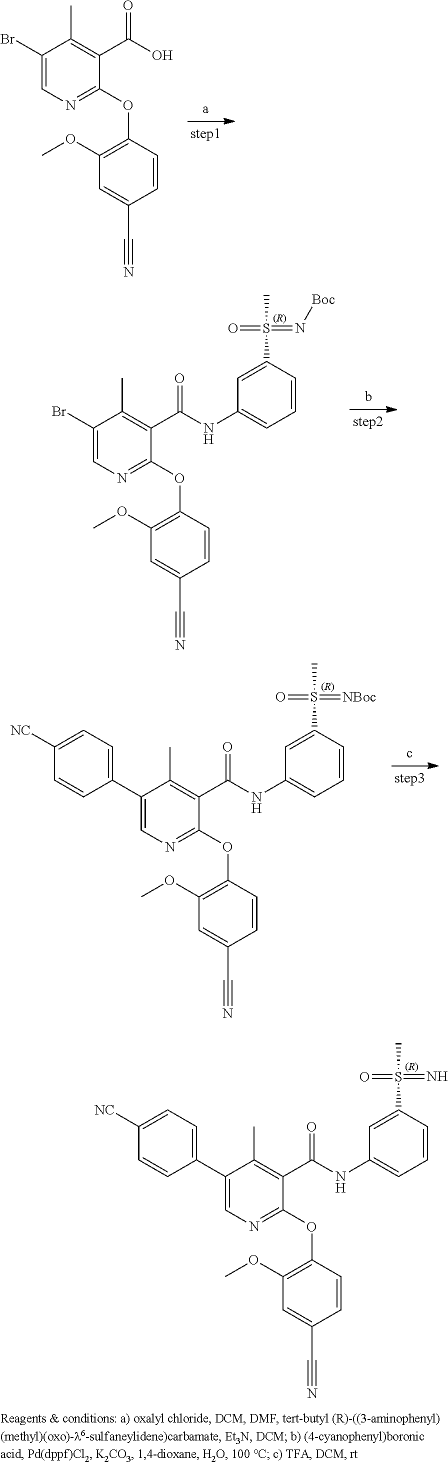

Step 1: tert-butyl (R)-((3-(5-bromo-2-(4-cyano-2-methoxyphenoxy)-4-methylnicotinamido)phenyl)(methyl)(oxo)-λ⁶-sulfaneylidene)carbamate: A solution of 5-bromo-2-(4-cyano-2-methoxyphenoxy)-4-methylnicotinic acid (420 mg, 1.16 mmol) in DCM (10 mL) was added DMF (one drop) and oxalyl chloride (297 mg, 2.32 mmol) at room temperature. The mixture was stirred at room temperature for 0.5 hour. Then the solution was concentrated under vacuum to provide the chloride intermediate. The chloride intermediate was dissolved in DCM (2 mL) and was added to a stirred solution of tert-butyl (R)-((3-aminophenyl)(methyl)(oxo)-λ⁶-sulfaneylidene)carbamate (314 mg, 1.16 mmol) and TEA (352 mg, 3.48 mmol) in DCM (5 mL) at 0° C. The resulting mixture was stirred at 25° C. for 1 hour. Then the mixture was quenched with water (40 mL) and extracted with DCM (30 mL×2). The combined organic phases were washed with brine, dried over sodium sulfate, concentrated under vacuum. The residue was purified by flash column chromatography on silica gel (PE/EtOAc=3/1) to provide tert-butyl (R)-((3-(5-bromo-2-(4-cyano-2-methoxyphenoxy)-4-methylnicotinamido)phenyl)(methyl)(oxo)-λ⁶-sulfaneylidene)carbamate (650 mg, 91%) as yellow solid. LCMS (ESI) calcd. for C₂₇H₂₇BrN₄O₆SNa [M+Na]⁺ m/z 637.08, found 637.05.

Step 2: tert-butyl (R)-((3-(2-(4-cyano-2-methoxyphenoxy)-5-(4-cyanophenyl)-4-methylnicotinamido)phenyl)(methyl)(oxo)-λ⁶-sulfaneylidene)carbamate: A mixture of tert-butyl (R)-((3-(5-bromo-2-(4-cyano-2-methoxyphenoxy)-4-methylnicotinamido)phenyl)(methyl)(oxo)-λ⁶-sulfaneylidene)carbamate (100 mg, 0.1628 mmol) and (4-cyanophenyl)boronic acid (120 mg, 0.8143 mmol) in 1,4-dioxane/H₂O (4/1, 10 mL), potassium carbonate (68 mg, 0.4884 mmol) and [1,1′-Bis(diphenylphosphino)ferrocene]dichloropalladium (II) (12 mg, 0.016 mmol) was heated at 100° C. for 6 hours under an atmosphere of N₂. The reaction was monitored by LCMS. After the reaction was completed, the mixture was cooled to room temperature. The resulting solution was diluted with water (20 mL) and extracted with DCM (20 mL×3). The combine organic phases were washed with brine, dried over Na₂SO₄, concentrated under vacuum. The residue was purified by flash column chromatography on silica gel (PE/EtAOc=1/1) to give tert-butyl (R)-((3-(2-(4-cyano-2-methoxyphenoxy)-5-(4-cyanophenyl)-4-methylnicotinamido)phenyl)(methyl)(oxo)λ⁶-sulfaneylidene)carbamate (72 mg, 69.90%) as a yellow oil. LCMS (ESI) calcd. for C₃₄H₃₂N₅O₆S [M+H]⁺ m/z 638.21, found 638.20.

Step 3: (R)-2-(4-cyano-2-methoxyphenoxy)-5-(4-cyanophenyl)-4-methyl-N-(3-(S-methylsulfonimidoyl)phenyl)nicotinamide: A solution of tert-butyl (R)-((3-(2-(4-cyano-2-methoxyphenoxy)-5-(4-cyanophenyl)-4-methylnicotinamido)phenyl)(methyl)(oxo)-λ⁶-sulfaneylidene)carbamate (72 mg, 0.113 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (10 mL×3). The combine organic phases were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% formic acid) to obtain (R)-2-(4-cyano-2-methoxyphenoxy)-5-(4-cyanophenyl)-4-methyl-N-(3-(S-methylsulfonimidoyl)phenyl)nicotinamide (15.7 mg, 26.17%) as a white solid. ¹H NMR (400 MHz, DMSO-d₆) δ 11.09 (s, 1H), 8.45-8.44 (m, 1H), 8.04 (s, 1H), 7.97 (d, J=8.4 Hz, 2H), 7.89-7.87 (m, 1H), 7.68-7.65 (m, 2H), 7.63-7.57 (m, 3H), 7.51-7.49 (m, 1H), 7.40 (d, J=8 Hz, 1H), 4.23 (s, 1H), 3.79 (s, 3H), 3.06 (s, 3H), 2.28 (s, 3H). LCMS (ESI) calcd. for C₂₉H₂₄N₅O₄S [M+H]⁺ m/z 538.16, found 538.20.

### Example 21C

### (R)-2-(4-cyano-2-methoxyphenoxy)-4-methyl-N-(3-(S-methylsulfonimidoyl)phenyl)-5-(p-tolyl)nicotinamide

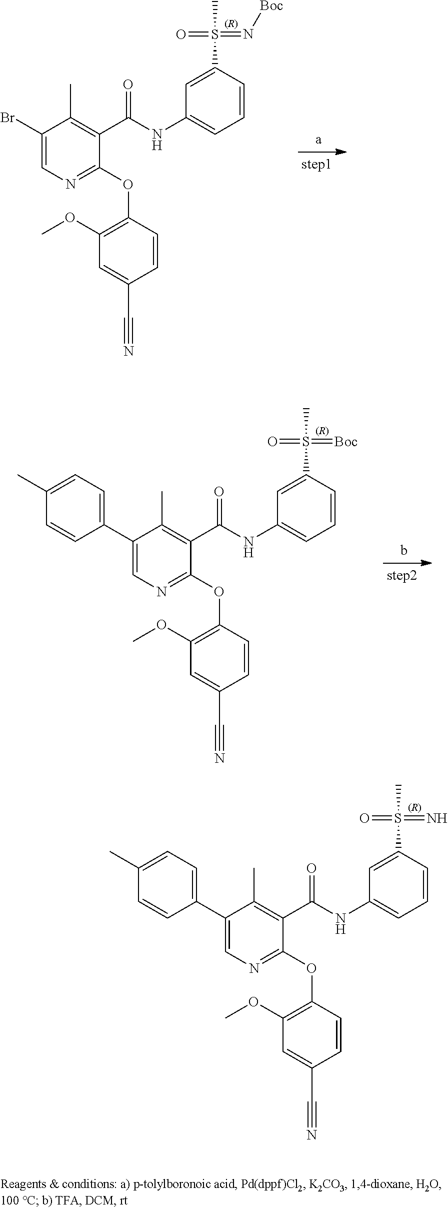

Step 1: tert-butyl (R)-((3-(2-(4-cyano-2-methoxyphenoxy)-4-methyl-5-(p-tolyl)nicotinamido)phenyl)(methyl)(oxo)-λ⁶-sulfaneylidene)carbamate: A mixture of tert-butyl (R)-((3-(5-bromo-2-(4-cyano-2-methoxyphenoxy)-4-methylnicotinamido)phenyl)(methyl)(oxo)-λ⁶-sulfaneylidene)carbamate (100 mg, 0.163 mmol), p-tolylboronic acid (66.37 mg, 0.488 mmol), potassium carbonate (67.48 mg, 0.49 mmol) and [1,1′-Bis(diphenylphosphino)ferrocene]dichloropalladium (II) (12 mg, 0.0163 mmol) in 1,4-dioxane/H₂O (4/1, 10 mL) was heated at 100° C. for 16 h under an atmosphere of N2. After the reaction was completed, the mixture was cooled to room temperature. The resulting solution was diluted with water (30 mL) and extracted with EtOAc (30 mL×3). The combined organic phases were washed with brine, dried over Na₂SO₄, concentrated under vacuum. The residue was purified by flash column chromatography on silica gel (PE/EtAOc=1/1) to give tert-butyl (R)-((3-(2-(4-cyano-2-methoxyphenoxy)-4-methyl-5-(p-tolyl)nicotinamido)phenyl)(methyl)(oxo)-λ⁶-sulfaneylidene)carbamate (60 mg, 58.8%) as a yellow oil. LCMS (ESI) calcd. for C₃₄H₃₅N₄O₆S [M+H]⁺ m/z 627.23, found 627.20.

Step 2: (R)-2-(4-cyano-2-methoxyphenoxy)-4-methyl-N-(3-(S-methylsulfonimidoyl)phenyl)-5-(p-tolyl)nicotinamide: A solution of tert-butyl (R)-((3-(2-(4-cyano-2-methoxyphenoxy)-4-methyl-5-(p-tolyl)nicotinamido)phenyl)(methyl)(oxo)-λ⁶-sulfaneylidene)carbamate (60 mg, 0.096 mmol) in DCM (3 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 45% to 95% MeCN/H₂O containing 0.05% NH₃) to provide (R)-2-(4-cyano-2-methoxyphenoxy)-4-methyl-N-(3-(S-methylsulfonimidoyl)phenyl)-5-(p-tolyl)nicotinamide (17.5 mg, 34.7%) as a white solid. ¹H NMR (400 MHz, DMSO-d₆) δ 11.07 (s, 1H), 8.46 (t, J=1.8 Hz, 1H), 7.97 (s, 1H), 7.90-7.87 (m, 1H), 7.66 (dd, J=9.2, 4.9 Hz, 2H), 7.59 (t, J=7.9 Hz, 1H), 7.50 (dd, J=8.2, 1.8 Hz, 1H), 7.39 (d, J=8.2 Hz, 1H), 7.30 (d, J=8.1 Hz, 2H), 7.26 (d, J=8.2 Hz, 2H), 4.23 (s, 1H), 3.79 (s, 3H), 3.06 (s, 3H), 2.36 (s, 3H), 2.27 (s, 3H). LCMS (ESI) calcd. for C₂₉H₂₇N₄O₄S [M+H]⁺ m/z 527.17, found 527.15.

### Example 22C

### (R)-6-(4-cyano-2-methoxyphenoxy)-4,5-dimethyl-N-(3-(S-methylsulfonimidoyl)phenyl)-[2,3-bipyridine]-5-carboxamide

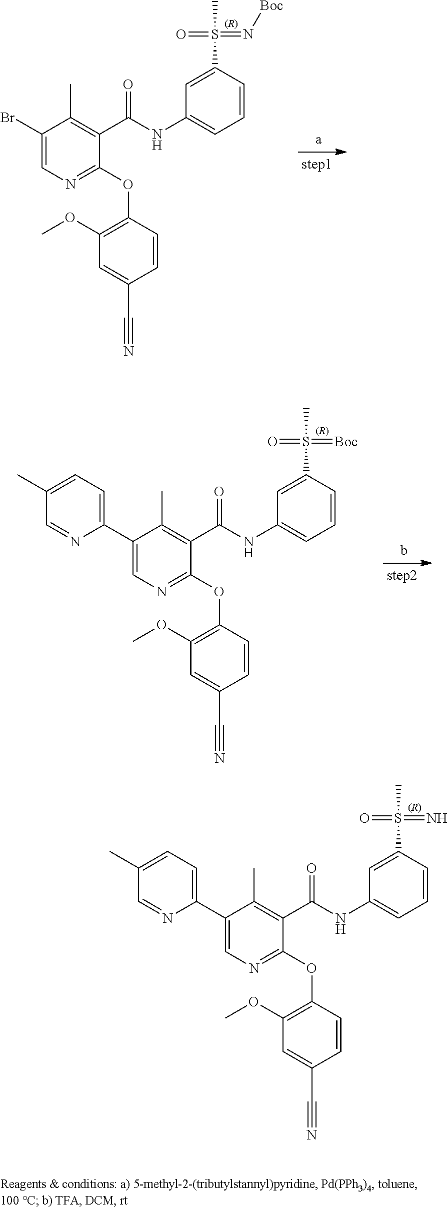

Step 1: tert-butyl (R)-((3-(6-(4-cyano-2-methoxyphenoxy)-4-dimethyl-[2,3-bipyridine]-5-carboxamido)phenyl)(methyl)(oxo)-λ⁶-sulfaneylidene)carbamate. A mixture of tert-butyl (R)-((3-(5-bromo-2-(4-cyano-2-methoxyphenoxy)-4-methylnicotinamido)phenyl)(methyl)(oxo)-λ⁶-sulfaneylidene)carbamate (100 mg, 0.163 mmol), 5-methyl-2-(tributylstannyl)pyridine (125 mg, 0.326 mmol) and Pd(PPh₃)₄ (19 mg, 0.016 mmol) in toluene (10 mL) was heated at 100° C. for 16 h under an atmosphere of N2. After the reaction was completed, the mixture was cooled to room temperature. The resulting solution was diluted with water (30 mL) and extracted with EtOAc (30 mL×3). The combined organic phases were washed with brine, dried over Na₂SO₄, concentrated under vacuum. The residue was purified by flash column chromatography on silica gel (PE/EtAOc=1/1) to give tert-butyl (R)-((3-(6-(4-cyano-2-methoxyphenoxy)-4,5-dimethyl-[2,3-bipyridine]-5-carboxamido)phenyl) (methyl)(oxo)-λ⁶-sulfaneylidene)carbamate (60 mg, 58.82%) as a yellow oil. LCMS (ESI) calcd. for C₃₃H₃₄N₅O₆S [M+H]⁺ m/z 628.23, found 628.15.

Step 2: (R)-6-(4-cyano-2-methoxyphenoxy)-4,5-dimethyl-N-(3-(S-methylsulfonimidoyl)phenyl)-[2,3-bipyridine]-5-carboxamide: A solution of tert-butyl (R)-((3-(6 (4-cyano-2-methoxyphenoxy)-4,5-dimethyl-[2,3-bipyridine]-5-carboxamido)phenyl)(methyl)(oxo)-λ⁶-sulfaneylidene)carbamate (60 mg, 0.096 mmol) in DCM (3 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini um C₁₈ column, 150*21.2 mm, eluting with 45% to 95% MeCN/H₂O containing 0.1% formic acid) to provide (R)-6-(4-cyano-2-methoxyphenoxy)-4-dimethyl-N-(3-(S-methylsulfonimidoyl)phenyl)-[2,3-bipyridine]-5-carboxamide (12.5 mg, 24.78%) as a white solid. ¹H NMR (400 MHz, DMSO-d₆) δ 11.09 (s, 1H), 8.55 (d, J=2.2 Hz, 1H), 8.46 (t, J=1.8 Hz, 1H), 8.12 (s, 1H), 7.92-7.86 (m, 1H), 7.75 (dd, J=8.0, 1.6 Hz, 1H), 7.70-7.64 (m, 2H), 7.59 (t, J=7.9 Hz, 1H), 7.52-7.46 (m, 2H), 7.39 (d, J=8.2 Hz, 1H), 4.24 (s, 1H), 3.79 (s, 3H), 3.06 (s, 3H), 2.36 (d, J=2.6 Hz, 6H). LCMS (ESI) calcd. for C₂₈H₂₆N₅O₄S [M+H]⁺ m/z 528.17, found 528.10.

### Example 23C

### (R)-2-(4-cyano-2-methoxyphenoxy)-5-(4-(difluoromethyl)phenyl)-4-methyl-N-(3-(S-methylsulfonimidoyl)phenyl)nicotinamide

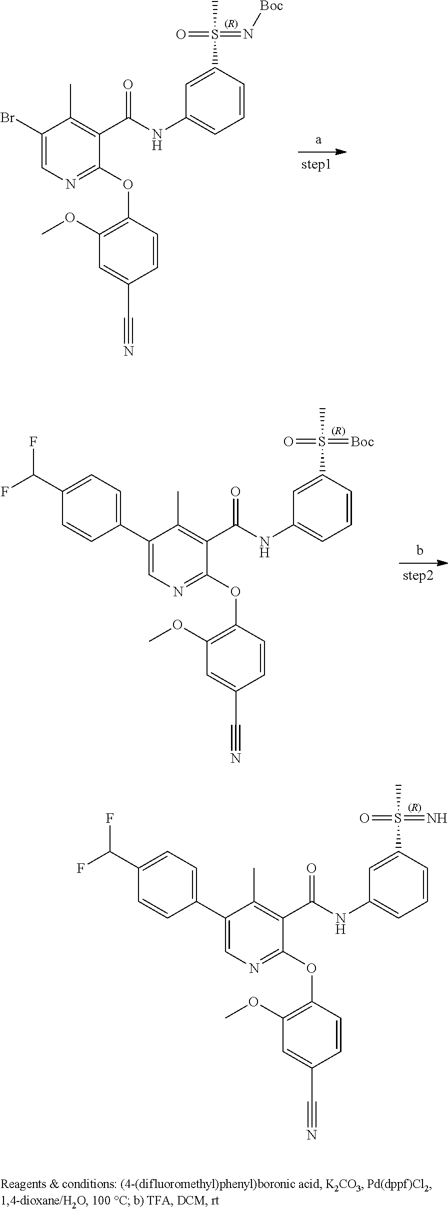

Step 1: tert-butyl (R)-((3-(2-(4-cyano-2-methoxyphenoxy)-5-(4-(difluoromethyl)phenyl)-4-methylnicotinamido)phenyl)(methyl)(oxo)-λ⁶-sulfaneylidene)carbamate: A mixture of tert-butyl (R)-((3-(5-bromo-2-(4-cyano-2-methoxyphenoxy)-4-methylnicotinamido)phenyl)(methyl) (oxo)-λ⁶-sulfaneylidene)carbamate (100 mg, 0.163 mmol), (4-(difluoromethyl)phenyl)boronic acid (56 mg, 0.326 mmol), potassium carbonate (67.48 mg, 0.49 mmol) and [1,1′-Bis(diphenylphosphino)ferrocene]dichloropalladium (II) (12 mg, 0.0163 mmol) in 1,4-dioxane/H₂O (4/1, 10 mL) was heated at 100° C. for 16 h under an atmosphere of N2. After the reaction was completed, the mixture was cooled to room temperature. The resulting solution was diluted with water (30 mL) and extracted with EtOAc (30 mL×3). The combined organic phases were washed with brine, dried over Na₂SO₄, concentrated under vacuum. The residue was purified by flash column chromatography on silica gel (PE/EtAOc=1/1) to give tert-butyl (R)-((3-(2-(4-cyano-2-methoxyphenoxy)-5-(4-(difluoromethyl)phenyl)-4-methylnicotinamido)phenyl) (methyl)(oxo)-λ⁶-sulfaneylidene)carbamate (80 mg, 74.0%) as a yellow oil. LCMS (ESI) calcd. for C₃₄H₃₂F₂N₄O₆SNa [M+Na]⁺ m/z 685.20, found 585.15.

Step 2: (R)-2-(4-cyano-2-methoxyphenoxy)-5-(4-(difluoromethyl)phenyl)-4-methyl-N-(3-(S-methylsulfonimidoyl)phenyl)nicotinamide: A solution of tert-butyl (R)-((3-(2-(4-cyano-2-methoxyphenoxy)-5-(4-(difluoromethyl)phenyl)-4-methylnicotinamido)phenyl)(methyl)(oxo)-λ⁶-sulfaneylidene)carbamate (80 mg, 0.121 mmol) in DCM (3 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 μm C₁₈ column, 150*21.2 mm, eluting with 45% to 95% MeCN/H₂O containing 0.1% formic acid) to provide (R)-2-(4-cyano-2-methoxyphenoxy)-5-(4-(difluoromethyl)phenyl)-4-methyl-N-(3-(S-methylsulfonimidoyl)phenyl)nicotinamide (21.3 mg, 31.37%) as a white solid. ¹H NMR (400 MHz, DMSO-d₆) δ 11.09 (s, 1H), 8.46 (s, 1H), 8.03 (s, 1H), 7.89 (d, J=8.1 Hz, 1H), 7.72-7.64 (m, 4H), 7.62-7.48 (m, 4H), 7.40 (d, J=8.2 Hz, 1H), 7.12 (t, J=55.9 Hz, 1H), 4.23 (s, 1 H), 3.79 (s, 3H), 3.06 (s, 3H), 2.28 (s, 3H). LCMS (ESI) calcd. for C₂₉H₂₅F₂N₄O₄S [M+H]⁺ m/z 563.16, found 563.10.

### Example 24C

### (R)-5-bromo-2-(4-cyano-2-methoxyphenoxy)-4-methyl-N-(3-(S-methylsulfonimidoyl)phenyl)nicotinamide

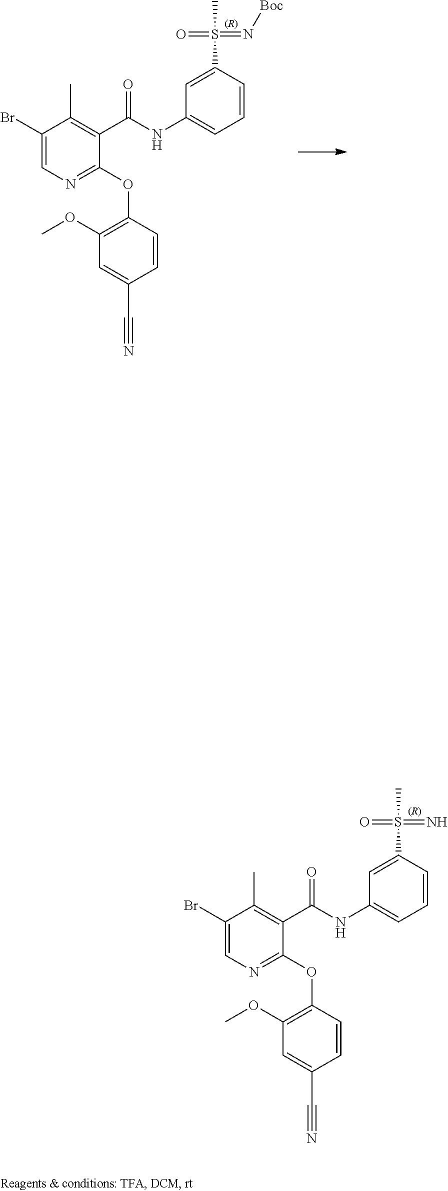

A solution of tert-butyl (R)-((3-(5-bromo-2-(4-cyano-2-methoxyphenoxy)-4-methylnicotinamido)phenyl)(methyl)(oxo)-λ⁶-sulfaneylidene)carbamate (60 mg, 0.096 mmol) in DCM (3 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 45% to 95% MeCN/H₂O containing 0.05% NH₃) to provide (R)-5-bromo-2-(4-cyano-2-methoxyphenoxy)-4-methyl-N-(3-(S-methylsulfonimidoyl)phenyl)nicotinamide (16.5 mg, 24.7%) as a white solid. ¹H NMR (400 MHz, DMSO-d₆) δ 11.06 (s, 1H), 8.40 (t, J=1.8 Hz, 1H), 8.30 (d, J=20.7 Hz, 1H), 7.87 (d, J=8.1 Hz, 1H), 7.68 (d, J=7.9 Hz, 1H), 7.65 (d, J=1.8 Hz, 1H), 7.60 (t, J=7.9 Hz, 1H), 7.49 (dd, J=8.2, 1.8 Hz, 1H), 7.36 (d, J=8.2 Hz, 1H), 4.23 (s, 1H), 3.76 (s, 3H), 3.06 (s, 3H), 2.41 (s, 3H). LCMS (ESI) calcd. for C₂₂H₂₀BrN₄O₄S [M+H]⁺ m/z 517.04, found 517.00.

### Example 25C

### 5-chloro-2-(4-cyano-2-methoxyphenoxy)-4-methyl-N-(3-(S-methylsulfonimidoyl)phenyl)nicotinamide

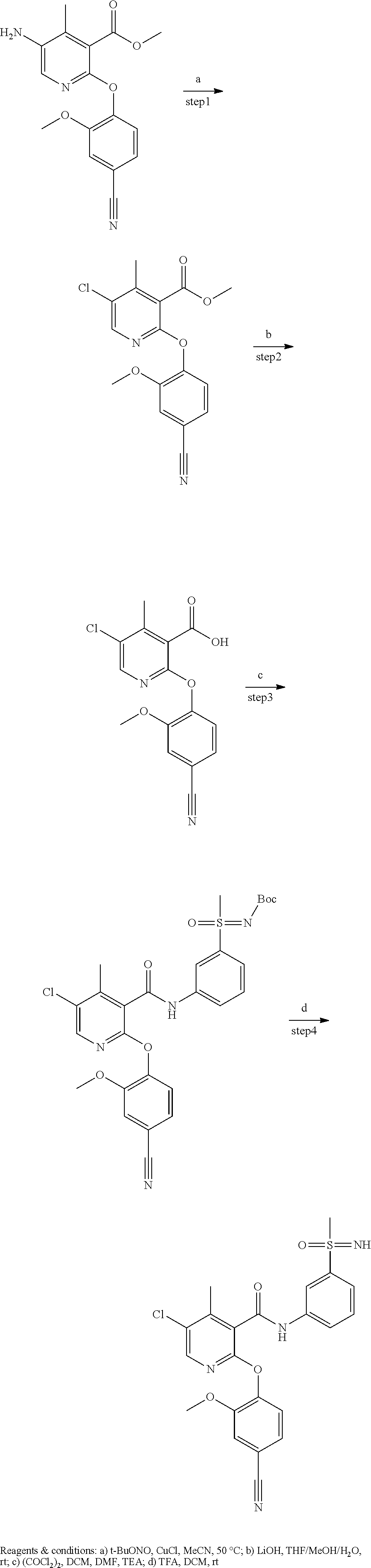

Step 1: methyl 5-chloro-2-(4-cyano-2-methoxyphenoxy)-4-methylnicotinate: To a solution of methyl 5-amino-2-(4-cyano-2-methoxyphenoxy)-4-methylnicotinate (500 mg, 1.6 mmol) and CuCl (158 mg, 1.6 mmol) in ACN (10 mL) was added t-BuONO (196 mg, 1.9 mmol) dropwise at room temperature. The reaction solution was stirred at 50° C. for 1 hour. After the reaction was completed, the resulting solution was diluted with water (20 mL) and extracted with EtOAc (30 mL×3). The combined organic phases were washed with brine, dried over sodium sulfate, concentrated under vacuum. The residue was purified by flash column chromatography on silica gel (PE/EtOAc=10/1) to give methyl 5-chloro-2-(4-cyano-2-methoxyphenoxy)-4-methylnicotinate (150 mg, 28%) as a yellow solid. LCMS (ESI) calcd. for C₁₆H₁₄C₁N₂O₄ [M+H]⁺ m/z 333.06, found 332.95.

Step 2: 5-chloro-2-(4-cyano-2-methoxyphenoxy)-4-methylnicotinic acid A solution of methyl 5-chloro-2-(4-cyano-2-methoxyphenoxy)-4-methylnicotinate (150 mg, 0.45 mmol) in THF (2 mL), MeOH (2 mL) and H₂O (2 mL) was added LiOH—H₂O (113 mg, 2.7 mmol) at room temperature. The reaction mixture was stirred at room temperature for 18 hours. After the reaction was completed, the mixture was concentrated. The residue was adjusted to pH=3-4 with 1N HCl. Then the aqueous solution was extracted with EtOAc (15 mL×3). The combined organic phases were washed with brine, dried over sodium sulfate, concentrated under vacuum to provide 5-chloro-2-(4-cyano-2-methoxyphenoxy)-4-methylnicotinic acid (100 mg, 49%) as a brown solid. LCMS (ESI) calcd. for C₁₅H₁₂ClN₂O₄ [M+H]⁺ m/z 319.05, found 318.90.

Step 3: tert-butyl ((3-(5-chloro-2-(4-cyano-2-methoxyphenoxy)-4-methylnicotinamido)phenyl)(methyl)(oxo)-λ⁶-sulfaneylidene)carbamate: A solution of 5-chloro-2-(4-cyano-2-methoxyphenoxy)-4-methylnicotinic acid (80 mg, 0.25 mmol) in DCM (3 mL) and DMF (one drop) was added oxalyl chloride (62 mg, 0.50 mmol). The solution was stirred at room temperature for 10 min. Then the formed chloride solution was added to a stirred solution of tert-butyl ((3-aminophenyl)(methyl)(oxo)-λ⁶-sulfaneylidene)carbamate (92 mg, 0.34 mmol) and DIPEA (88 mg, 0.68 mmol) in DCM (2 mL) at 0° C. The resulting mixture was stirred at 25° C. for 1 h. Then the mixture was quenched with water (20 mL) and extracted with DCM (20 mL×2). The combined organic phases were washed with brine, dried over sodium sulfate, concentrated under vacuum. The residue was purified by flash column chromatography on silica gel (PE/EtOAc=1/1) to provide tert-butyl ((3-(5-chloro-2-(4-cyano-2-methoxyphenoxy)-4-methylnicotinamido)phenyl)(methyl)(oxo)-λ⁶-sulfaneylidene)carbamate (60 mg, 42%) as a white oil. LCMS (ESI) calcd. for C₂₇H₂₇ClN₄O₆SNa [M+Na]⁺ m/z 593.12, found 593.10.

Step 4: 5-chloro-2-(4-cyano-2-methoxyphenoxy)-4-methyl-N-(3-(S-methylsulfonimidoyl)phenyl)nicotinamide: A solution of tert-butyl ((3-(5-chloro-2-(4-cyano-2-methoxyphenoxy)-4-methylnicotinamido)phenyl)(methyl)(oxo)-λ⁶-sulfaneylidene)carbamate (60 mg, 0.105 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (30 mL×3). The combined organic phases were washed with brine, dried over sodium sulfate, concentrated under vacuum. The residue was purified by prep-HPLC (Gemini 5 um C₁₈ column, 150*21.2 mm, eluting with 50% to 85% McCN/H₂O containing 0.05% NH₃) to provide 5-chloro-2-(4-cyano-2-methoxyphenoxy)-4-methyl-N-(3-(S-methylsulfonimidoyl)phenyl)nicotinamide (25 mg, 51%) as a white solid. ¹H NMR (400 MHz, DMSO-d₆) δ 11.06 (s, 1H), 8.40 (t, J=1.8 Hz, 1H), 8.23 (s, 1H), 7.88 (d, J=8.9 Hz, 1H), 7.64 (m, 3H), 7.49 (dd, J=8.2, 1.8 Hz, 1H), 7.37 (d, J=8.2 Hz, 1H), 4.23 (s, 1H), 3.76 (s, 3H), 3.06 (s, 3H), 2.40 (s, 3H). LCMS (ESI) calcd. for C₂₂H₂₀ClN₄O₄S [M+H]⁺ m/z 471.09, found 471.05.

### Example 26C

### (R)-5-chloro-2-(4-cyano-2-methoxyphenoxy)-4-methyl-N-(3-(S-methylsulfonimidoyl)phenyl)nicotinamide

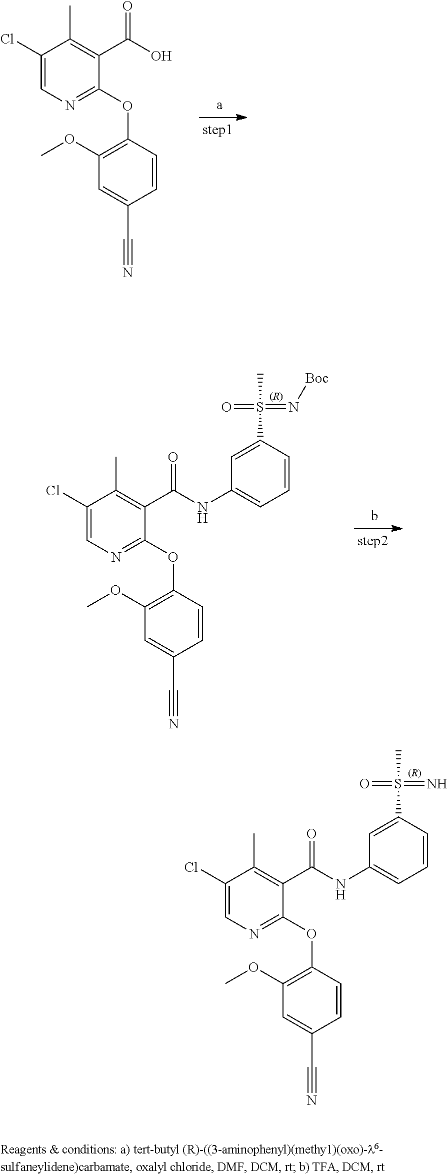

Step 1: tert-butyl (R)-((3-(5-chloro-2-(4-cyano-2-methoxyphenoxy)-4-methylnicotinamido)phenyl)(methyl)(oxo)-λ⁶-sulfaneylidene)carbamate: A solution of 5-chloro-2-(4-cyano-2-methoxyphenoxy)-4-methylnicotinic acid (150 mg, 0.47 mmol) in DCM (3 mL) and DMF (one drop) was added oxalyl chloride (120 mg, 1 mmol). The solution was stirred at room temperature for 5 min. Then the solution was added to a stirred solution of tert-butyl (R)-((3-aminophenyl)(methyl)(oxo)-λ⁶-sulfaneylidene)carbamate (180 mg, 0.68 mmol) and TEA (141 mg, 1.39 mmol) in DCM (3 mL) at 0° C. The resulting mixture was stirred at 25° C. for 1 h. Then the mixture was quenched with water (20 mL) and extracted with DCM (20 mL×2). The combined organic phases were washed with brine, dried over sodium sulfate, concentrated under vacuum. The residue was purified by flash column chromatography on silica gel (PE/EtOAc=1/1) to provide tert-butyl (R)-((3-(5-chloro-2-(4-cyano-2-methoxyphenoxy)-4-methylnicotinamido)phenyl)(methyl)(oxo)-λ⁶-sulfaneylidene)carbamate (85 mg, 32% yield) as a white oil. LCMS (ESI) calcd. for C₂₇H₂₇ClN₄O₆SNa [M+Na]⁺ m/z 593.08, found 593.10.

Step 2: (R)-5-chloro-2-(4-cyano-2-methoxyphenoxy)-4-methyl-N-(3-(S-methylsulfonimidoyl)phenyl)nicotinamide: A solution of tert-butyl (R)-((3-(5-chloro-2-(4-cyano-2-methoxyphenoxy)-4-methylnicotinamido)phenyl)(methyl)(oxo)-λ⁶-sulfaneylidene)carbamate (85 mg, 0.15 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (30 mL×3). The combined organic phases were washed with brine, dried over sodium sulfate, concentrated under vacuum. The residue was purified by prep-HPLC (Gemini 5 um C₁₈ column, 150*21.2 mm, eluting with 50% to 85% MeCN/H₂O containing 0.05% NH₃) to provide (R)-5-chloro-2-(4-cyano-2-methoxyphenoxy)-4-methyl-N-(3-(S-methylsulfonimidoyl)phenyl)nicotinamide (42.3 mg, 60.4%) as a white solid. LCMS (ESI) calcd. for C₂₂H₂₀ClN₄O₄S [M+H]⁺ m/z 471.09, found 471.00. ¹H NMR (400 MHz, DMSO-d₆) δ 11.06 (s, 1H), 8.40 (t, J=1.8 Hz, 1H), 8.23 (s, 1H), 7.88 (d, J=8.9 Hz, 1H), 7.64 (m, 3H), 7.49 (dd, J=8.2, 1.8 Hz, 1H), 7.37 (d, J=8.2 Hz, 1H), 4.23 (s, 1H), 3.76 (s, 3H), 3.06 (s, 3H), 2.40 (s, 3H). C₂₃H₂₈F₄N₅O₂S [M+H]⁺ m/z 514.18, found 514.10.

### Example 27C

### (R)-2-(4-cyano-2-methoxyphenoxy)-5-(1,3-dimethyl-1H-pyrazol-4-yl)-4-methyl-N-(3-(S-methylsulfonimidoyl)phenyl)nicotinamide

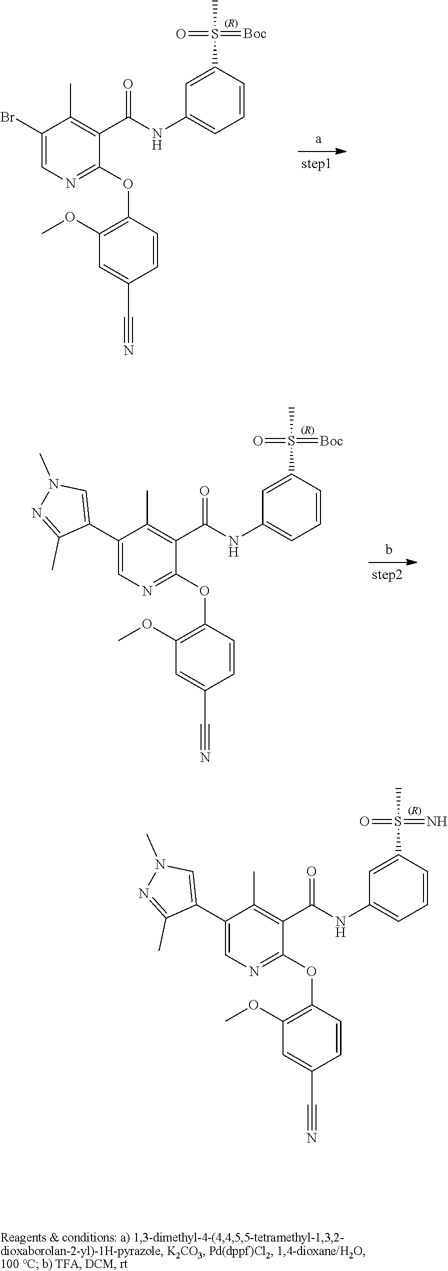

Step 1: tert-butyl (R)-((3-(2-(4-cyano-2-methoxyphenoxy)-5-(1,3-dimethyl-1H-pyrazol-4-yl)-4-methylnicotinamido)phenyl)(methyl)(oxo)-l6-sulfaneylidene)carbamate: A mixture of tert-butyl (R)-((3-(5-bromo-2-(4-cyano-2-methoxyphenoxy)-4-methylnicotinamido)phenyl)(methyl)(oxo)-λ⁶-sulfaneylidene)carbamate (100 mg, 0.163 mmol) and 1,3-dimethyl-4-(4,4,5,5-tetramethyl-1,3,2-dioxaborolan-2-yl)-1H-pyrazole (55 mg, 0.244 mmol) in 1,4-dioxane/H₂O (4/1, 10 mL), K₂CO₃ (70 mg, 0.489 mmol) and [1,1′-Bis(diphenylphosphino)ferrocene]dichloropalladium (II) (20 mg, 0.016 mmol) was heated at 100° C. for 6 hours under an atmosphere of N2. The reaction was monitored by LCMS. After the reaction was completed, the mixture was cooled to room temperature. The resulting solution was diluted with water (20 mL) and extracted with DCM (20 mL×3). The combined organic layers were washed with brine, dried over Na₂SO₄, and concentrated under vacuum. The residue was purified by flash column chromatography on silica gel (PE/EtOAc=2/1 to 1/1) to give tert-butyl (R)-((3-(2-(4-cyano-2-methoxyphenoxy)-5-(1,3-dimethyl-1H-pyrazol-4-yl)-4-methylnicotinamido)phenyl)(methyl)(oxo)-l6-sulfaneylidene)carbamate (80 mg, 78.43%) as a yellow oil. LCMS (ESI) calcd. for C₃₂H₃₅N₆O₆S [M+H]⁺ m/z 631.23, found 631.1 Step 2: (R)-2-(4-cyano-2-methoxyphenoxy)-5-(1,3-dimethyl-1H-pyrazol-4-yl)-4-methyl-N-(3-(S-methylsulfonimidoyl)phenyl)nicotinamide: A solution of tert-butyl (R)-((3-(2-(4-cyano-2-methoxyphenoxy)-5-(1,3-dimethyl-1H-pyrazol-4-yl)-4-methylnicotinamido)phenyl)(methyl) (oxo)-λ⁶-sulfaneylidene)carbamate (80 mg, 0.127 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.1% formic acid) to obtain (R)-2-(4-cyano-2-methoxyphenoxy)-5-(1,3-dimethyl-1H-pyrazol-4-yl)-4-methyl-N-(3-(S-methylsulfonimidoyl)phenyl)nicotinamide (34.8 mg, 51.94%) as a white solid. ¹H NMR (400 MHz, DMSO-d₆) δ 11.06 (s, 1H), 8.45 (s, 1H), 7.93 (s, 1H), 7.89 (d, J=8.0 Hz, 1H), 7.75 (s, 1H), 7.66-7.63 (m, 2H), 7.61-7.57 (m, 1H),), 7.50-7.48 (m, 1H),), 7.39-7.37 (m, 1H), 4.23 (s, 1H), 3.81 (s, 3H), 3.78 (s, 3H), 3.06 (s, 3H), 2.23 (s, 3H), 2.07 (s, 3H). LCMS (ESI) calcd. for C₂₇H₂₇N₆O₄S [M+H]⁺ m/z 531.17, found 531.15.

### Example 28C

### 2-(4-cyano-2-methoxyphenoxy)-4-methyl-N-(3-sulfamoylphenyl)-5-(trifluoromethyl)nicotinamide

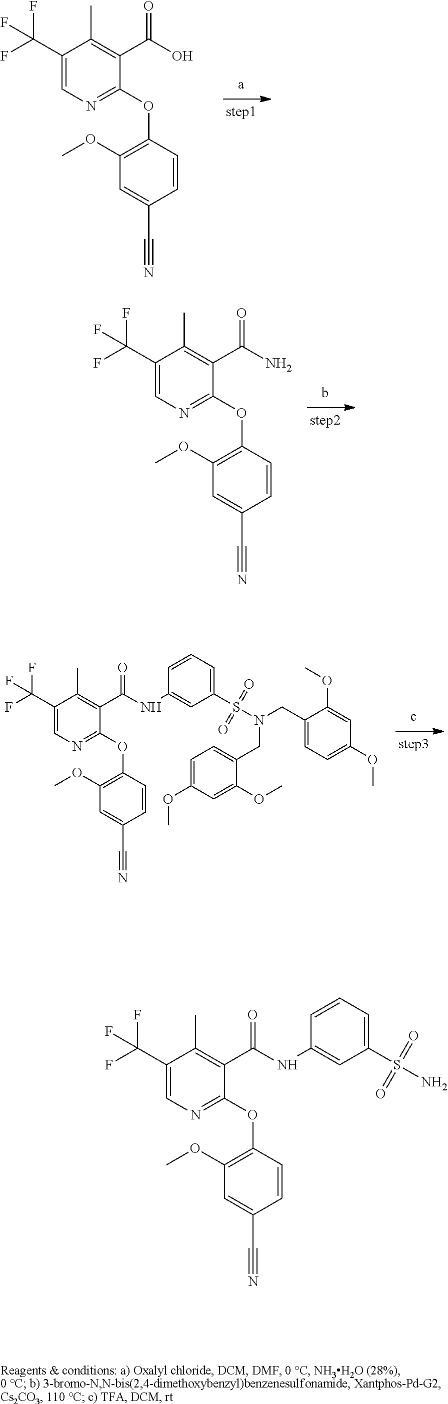

Step 1: 2-(4-cyano-2-methoxyphenoxy)-4-methyl-5-(trifluoromethyl)nicotinamide: A solution of 2-(4-cyano-2-methoxyphenoxy)-4-methyl-5-(trifluoromethyl)nicotinic acid (140 mg, 0.39 mmol) in DCM (5 mL) was added DMF (4 drops) and Oxalyl chloride (75 mg, 0.59 mmol) at room temperature. The reaction mixture was stirred at room temperature for 10 minutes. Then the reaction was cooled to 0° C. and added 28% NH₃·H₂O (0.5 mL). The reaction mixture was stirred at room temperature for 2 hours. After the reaction was completed, the resulting solution was diluted with water (10 mL) and extracted with DCM (10 mL×3). The combined organic phases were washed with brine, dried over sodium sulfate, concentrated under vacuum. The residue was purified by flash column chromatography on silica gel (DCM/MeOH=10/1) to give 2-(4-cyano-2-methoxyphenoxy)-4-methyl-5-(trifluoromethyl)nicotinamide (100 mg, 71.68%). LCMS (ESI) calcd. for C₁₆H₁₃F₃N₃O₃ [M+H]⁺ m/z 352.09, found 352.00.

Step 2: N-(3-(N,N-bis(2,4-dimethoxybenzyl)sulfamoyl)phenyl)-2-(4-cyano-2-methoxyphenoxy)-4-methyl-5-(trifluoromethyl)nicotinamide: A solution of 2-(4-cyano-2-methoxyphenoxy)-4-methyl-5-(trifluoromethyl)nicotinamide (200 mg, 0.57 mmol), 3-bromo-N,N-bis(2,4-dimethoxybenzyl)benzenesulfonamide (1.2 g, 2.28 mmol), Cs₂CO₃ (926 mg, 2.85 mmol) and Xantphos-Pd-G2 (202 mg, 0.22 mmol) in dioxane (20 mL) was heated at 110° C. for 8 hours. LCMS showed the reaction was completed. The mixture was diluted with water (20 mL) and extracted with EtOAc (20 mL×3). The combined organic layers were washed with brine, dried over sodium sulfate, concentrated under vacuum. The residue was purified by flash column chromatography on silica gel (PE/EtOAc=1/1) to provide N-(3-(N,N-bis(2,4-dimethoxybenzyl)sulfamoyl)phenyl)-2-(4-cyano-2-methoxyphenoxy)-4-methyl-5-(trifluoromethyl)nicotinamide (115 mg, 25%) as yellow solid. LCMS (ESI) calcd. for C₄₀H₃₇F₃N₄O₉SNa [M+Na]⁺ m/z 829.21, found 829.20.

Step 3: 2-(4-cyano-2-methoxyphenoxy)-4-methyl-N-(3-sulfamoylphenyl)-5-(trifluoromethyl)nicotinamide: A solution of N-(3-(N,N-bis(2,4-dimethoxybenzyl)sulfamoyl)phenyl)-2-(4-cyano-2-methoxyphenoxy)-4-methyl-5-(trifluoromethyl)nicotinamide (115 mg, 0.14 mmol) in DCM (2 mL) was added TFA (1 mL) at room temperature. The reaction mixture was stirred at room temperature for 1 hour. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 40% to 95% MeCN/H₂O containing 0.05% NH₃H₂O) to provide 2-(4-cyano-2-methoxyphenoxy)-4-methyl-N-(3-sulfamoylphenyl)-5-(trifluoromethyl)nicotinamide (47.66 mg, 66.03%) as a white solid. ¹H NMR (400 MHz, DMSO-d₆) δ 11.11 (s, 1H), 8.52 (s, 1H), 8.35 (d, J=1.6 Hz, 1H), 7.79 (dt, J=6.6, 2.3 Hz, 1H), 7.68 (d, J=1.8 Hz, 1H), 7.61-7.57 (m, 2H), 7.52 (dd, J=8.2, 1.8 Hz, 1H), 7.44-7.38 (m, 3H), 3.77 (s, 3H), 2.48 (s, 3H). LCMS (ESI) calcd. for C₂₂H₁₈F₃N₄O₅S [M+H]⁺ m/z 507.10, found 507.00.

### Example 29C

### 4-(4-cyano-2-methylphenoxy)-N-(3-(methylsulfonyl)phenyl)-6-(trifluoromethyl)pyridazine-3-carboxamide

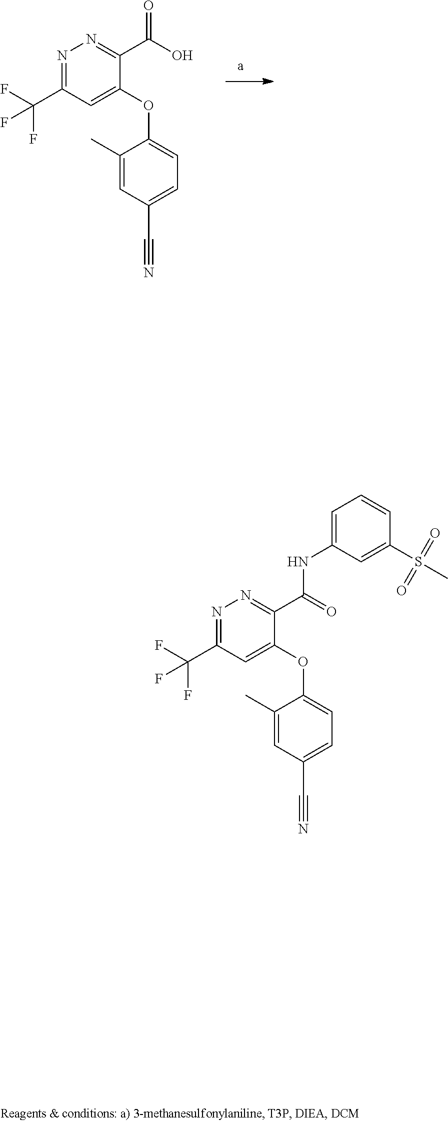 [SMILES: Cc1cc(C#N)ccc1Oc1cc(C(F)(F)F)nnc1C(=O)O ; Cc1cc(C#N)ccc1Oc1cc(C(F)(F)F)nnc1C(=O)Nc1cccc(S(C)(=O)=O)c1]

To a solution of 4-(4-cyano-2-methylphenoxy)-6-(trifluoromethyl)pyridazine-3-carboxylic acid (120 mg, 0.37 mmol), 3-methanesulfonylaniline (64 mg, 0.37 mmol) and T3P (472 mg, 50% in EtOAc) in DCM (5 mL) was added DIEA (287 mg, 2.23 mmol). The mixture was stirred at room temperate for 2 hours. The resulting mixture was diluted with water (10 mL) and extracted with EtOAc (10 mL×3). The combine organic phases were washed with brine, dried over sodium sulfate, concentrated under vacuum. The residue was first purified by flash column chromatography on silica gel (eluting with EtOAc/PE, 0% to 90%) to afford 4-(4-cyano-2-methylphenoxy)-N-(3-(methylsulfonyl)phenyl)-6-(trifluoromethyl)pyridazine-3-carboxamide (16.05 mg, 9.08%) as a light pink solid. ¹H NMR (400 MHz, DMSO-d₆) δ 11.49 (s, 1H), 8.42 (s, 1H), 8.01-7.94 (m, 2H), 7.83 (dd, J=8.2, 1.6 Hz, 1H), 7.77-7.67 (m, 3H), 7.47 (d, J=8.5 Hz, 1H), 3.24 (s, 3H), 2.23 (s, 3H). LCMS (ESI) calcd. for C₂₁H₁₆F₃N₄O₄S [M+H]⁺ m/z 477.08, found 477.20.

### Example 30C

### 4-(3,4-difluoro-2-methoxyphenoxy)-N-(3-(methylsulfinyl) phenyl)-6-(trifluoromethyl)pyridazine-3-carboxamide

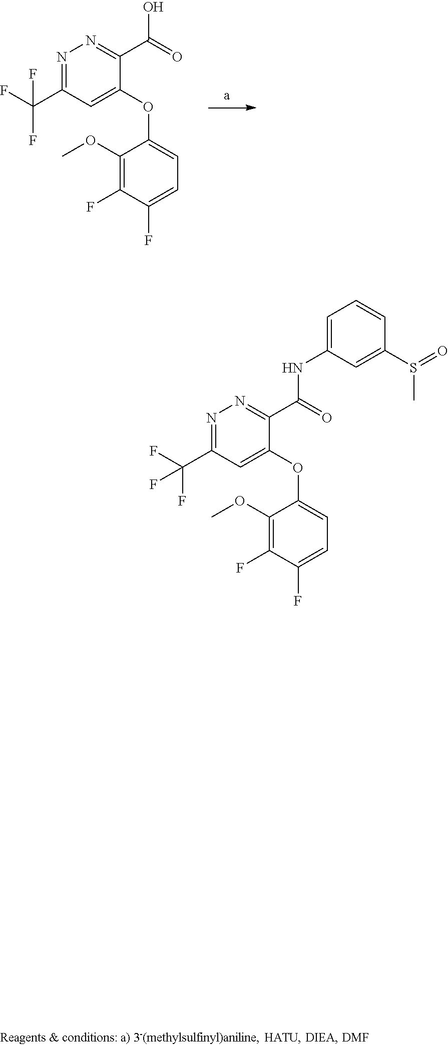

To a solution of 4-(3,4-difluoro-2-methoxyphenoxy)-6-(trifluoromethyl)pyridazine-3-carboxylic acid (100 mg, 0.29 mmol), 3-(methylsulfinyl) aniline (53 mg, 0.34 mmol) and HATU (217 mg, 0.57 mmol) in DMF (2 mL) was added DIEA (110 mg, 0.86 mmol). The mixture was stirred at room temperate for 1 hours. The mixture was diluted with water (10 mL) and extracted with EtOAc (10 mL×3). The combine organic phases were washed with brine, dried over sodium sulfate, concentrated under vacuum. The residue was purified by flash column chromatography on silica gel (PE/EtOAc=5/1 to 2/1) to afford 4-(3,4-difluoro-2-methoxyphenoxy)-N-(3-(methylsulfinyl) phenyl)-6-(trifluoromethyl) pyridazine-3-carboxamide (120 mg, 86.33%) as a yellow solid. ¹H NMR (400 MHz, DMSO-d₆) δ 11.39 (s, 1H), 8.19 (s, 1H), 7.91 (s, 1H), 7.83 (d, J=9.0 Hz, 1H), 7.60 (t, J=7.9 Hz, 1H), 7.46 (d, J=7.8 Hz, 1H), 7.36-7.28 (m, 1H), 7.14 (t, J=9.0 Hz, 1 H), 3.91 (s, 3H), 2.77 (s, 3H). LCMS (ESI) calcd. for C₂₀H₁₅F₅N₃O₄S [M+H]⁺ m/z 488.07, found 487.95.

### Example 31C

### 4-(3,4-difluoro-2-methoxyphenoxy)-N-(3-(S-methylsulfonimidoyl)phenyl)-6-(trifluoromethyl)pyridazine-3-carboxamide

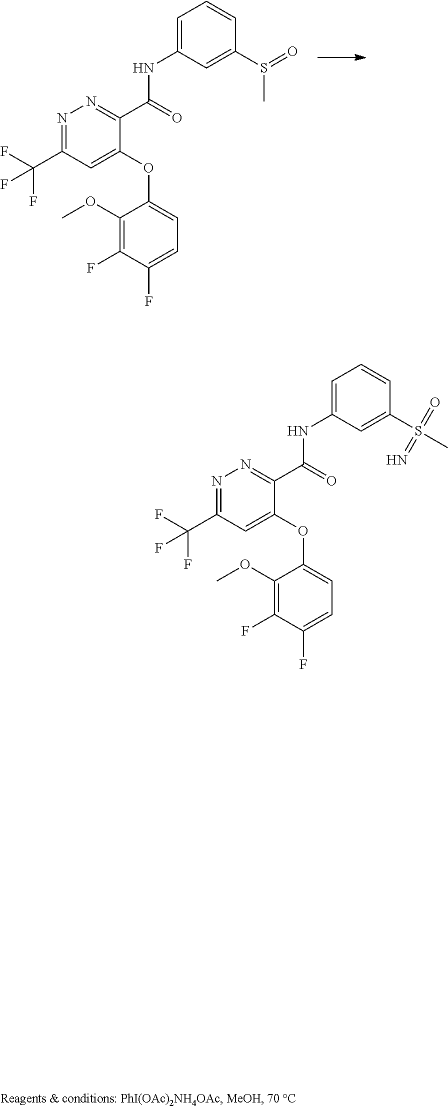

A solution of 4-(3,4-difluoro-2-methoxyphenoxy)-N-(3-(methylsulfinyl)phenyl)-6-(trifluoromethyl)pyridazine-3-carboxamide (120 mg, 0.25 mmol), NH₄OAc (95 mg, 1.23 mmol) and PhI(OAc)₂ (396 mg, 1.23 mmol) in MeOH (5 mL) was heated at 70° C. for 4 hours. The mixture was concentrated under vacuum. The residue was purified by prep-HPLC (Gemini 5 um C₁₈ column, 150*21.2 mm, eluting with 30% to 90% MeCN/H₂O containing 0.1% formic acid) to afford 4-(3,4-difluoro-2-methoxyphenoxy)-N-(3-(S-methylsulfonimidoyl)phenyl)-6-(trifluoromethyl)pyridazine-3-carboxamide (21 mg, 17.07%) as a white solid. ¹H NMR (400 MHz, DMSO-d₆) δ 11.44 (s, 1H), 8.44 (s, 1H), 7.99-7.87 (m, 2H), 7.76-7.61 (m, 2H), 7.32 (t, J=8.9 Hz, 1H), 7.15 (t, J=9.0 Hz, 1H), 4.27 (s, 1H), 3.91 (s, 3H), 3.08 (s, 3H). LCMS (ESI) calcd. for C₂₀H₁₆F₅N₄O₄S [M+H]⁺ m/z 503.08, found 502.95.

### Example 32C

### 4-(3,4-difluoro-2-methoxyphenoxy)-N-(3-(methylsulfonyl)phenyl)-6-(trifluoromethyl)pyridazine-3-carboxamide

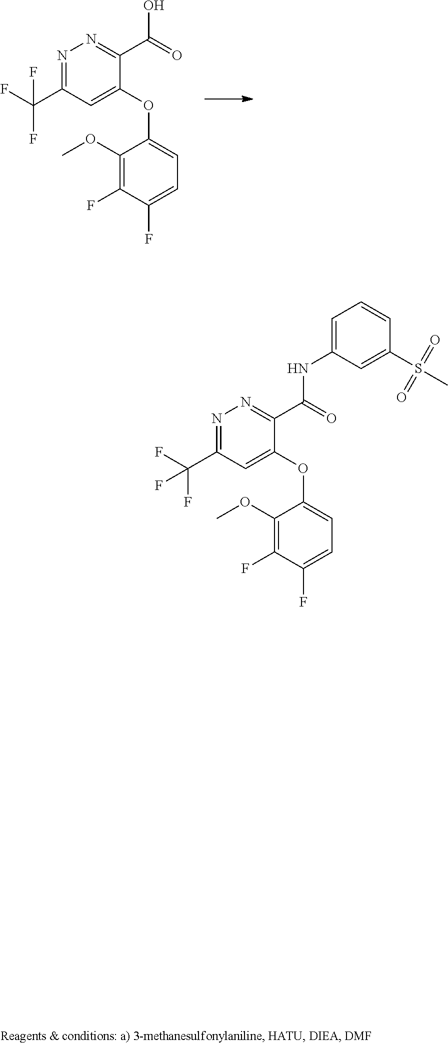

To a solution of 4-(3,4-difluoro-2-methoxyphenoxy)-6-(trifluoromethyl)pyridazine-3-carboxylic acid (100 mg, 0.29 mmol), 3-(methylsulfonyl) aniline (59 mg, 0.34 mmol) and HATU (217 mg, 0.57 mmol) in DMF (2 mL) was added DIEA (110 mg, 0.86 mmol). The mixture was stirred at room temperate for 1 hours. Then the mixture was diluted with water (10 mL) and extracted with EtOAc (10 mL×3). The combine organic phases were washed with brine, dried over sodium sulfate, concentrated under vacuum. The residue was first purified by flash column chromatography on silica gel (PE/EtOAc=3/1), then further purified by prep-HPLC (Gemini 5 um C₁₈ column, 150*21.2 mm, eluting with 30% to 90% MeCN/H₂O containing 0.1% formic acid) to afford 4-(3,4-difluoro-2-methoxyphenoxy)-N-(3-(methylsulfonyl)phenyl)-6-(trifluoromethyl)pyridazine-3-carboxamide (33 mg, 23.08%) as a white solid. ¹H NMR (400 MHz, DMSO-d₆) δ 11.51 (s, 1H), 8.45 (s, 1H), 8.00 (d, J=7.8 Hz, 1H), 7.92 (s, 1H), 7.72 (dt, J=15.6, 7.9 Hz, 2H), 7.32 (t, J=8.8 Hz, 1H), 7.14 (t, J=8.9 Hz, 1H), 3.91 (s, 3H), 3.24 (s, 3H). LCMS (ESI) calcd. for C₂₀H₁₅F₅N₃O₅S [M+H]⁺ m/z 504.07, found 504.00.

### Example 33C

### (R)—N-(3-(N,S-dimethylsulfonimidoyl)phenyl)-2-((6-fluoro-2-methylpyridin-3-yl)oxy)-4-methyl-5-(trifluoromethyl)nicotinamide

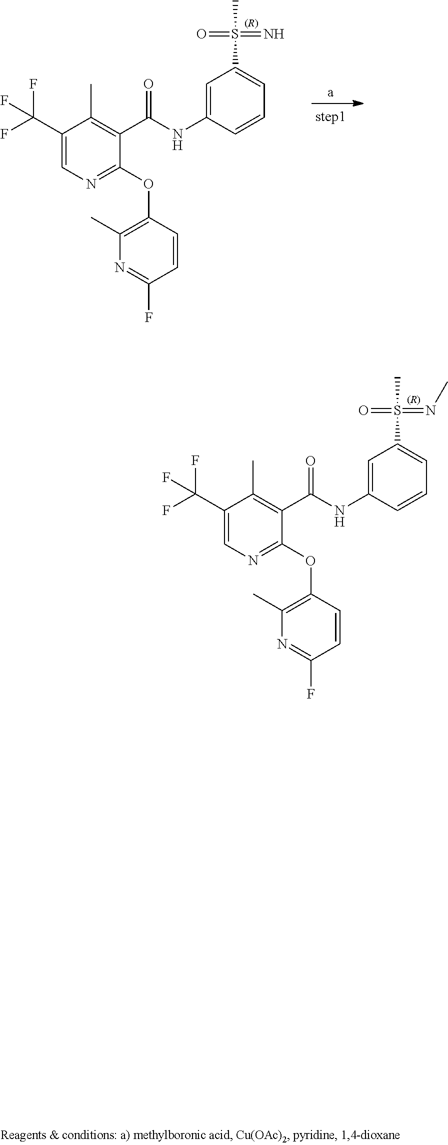

A mixture of (R)-2-((6-fluoro-2-methylpyridin-3-yl)oxy)-4-methyl-N-(3-(S-methylsulfonimidoyl)phenyl)-5-(trifluoromethyl)nicotinamide (See. WO2022/192487; 100 mg, 0.21 mmol), Cu(OAc)₂ (63 mg, 0.32 mmol.), pyridine (33 mg, 0.42 mmol) in 1, 4-dioxane (8 mL) was stirred for 5 min at room temperature under open air condition. Then the mixture was added methylboronic acid (25 mg, 0.42 mmol). The resulting mixture was heated at 100° C. for 16 hours. After the reaction was completed, the mixture was cooled to room temperature. The mixture was diluted with water (30 mL) and extracted with EA (20 mL×3). The combined organic layers were washed with brine, dried over Na₂SO₄, and concentrated under vacuum. The residue was first purified by flash column chromatography on silica gel (PE/EtOAc=2/1 to 1/1) then further purified by prep-HPLC (Gemini 5 um C₁₈ column, 150*21.2 mm, eluting with 40% to 90% MeCN/H₂O containing 0.05% NH₃) to provide (R)—N-(3-(N,S-dimethylsulfonimidoyl)phenyl)-2-((6-fluoro-2-methylpyridin-3-yl)oxy)-4-methyl-5-(trifluoromethyl)nicotinamide (47.5 mg, 45.5) as a white solid. ¹H NMR (400 MHz, CD₃OD) δ 8.44 (s, 1H), 8.39 (d, J=1.0 Hz, 1H), 7.99 (dd, J=4.3, 2.3 Hz, 1H), 7.81-7.65 (m, 3H), 6.99 (dd, J=8.6, 2.8 Hz, 1H), 3.20 (s, 3H), 2.66-2.55 (m, 6H), 2.32 (s, 3H). LCMS (ESI) calcd. for C₂₂H₂₁F₄N₄O₃S [M+H]⁻ m/z 497.13, found 497.00.

### Example 34C

### 4-(4-cyano-2-methoxyphenoxy)-N-(3-(S-methylsulfonimidoyl)phenyl)-6-(trifluoromethyl)pyridazine-3-carboxamide

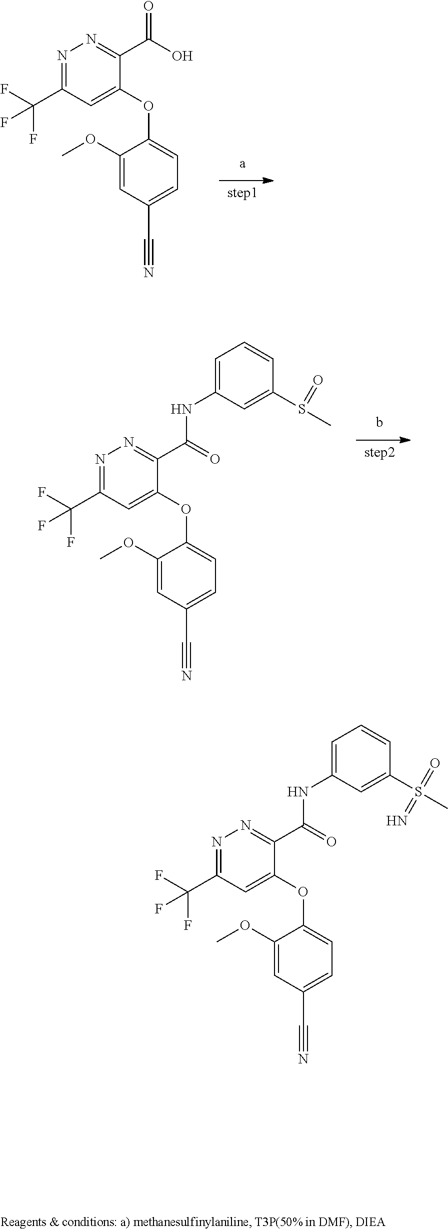

Step 1: 4-(4-cyano-2-methoxyphenoxy)-N-(3-(methylsulfinyl)phenyl)-6-(trifluoromethyl)pyridazine-3-carboxamide: To a solution of 4-(4-cyano-2-methoxyphenoxy)-6-(trifluoromethyl)pyridazine-3-carboxylic acid (130 mg, 0.38 mmol) and 3-methanesulfinylaniline (59 mg, 0.38 mmol) in DCM (5 mL) was added T3P (583 mg, 50% in DMF) and DIEA (297 mg, 2.30 mmol). The mixture was stirred at room temperate for 2 hours. The mixture was diluted with water (20 mL) and extracted with EtOAc (20 mL×3). The combine organic phases were washed with brine, dried over sodium sulfate, concentrated under vacuum. The residue was first purified by flash column chromatography on silica gel (eluting with EtOAc/PE, 0% to 90%) to give 4-(4-cyano-2-methoxyphenoxy)-N-(3-(methylsulfinyl)phenyl)-6-(trifluoromethyl)pyridazine-3-carboxamide (90 mg, 49.31%) as an off-white solid. LCMS (ESI) calcd. for C₂₁H₁₆F₃N₄O₄S [M+H]⁺ m/z 477.08, found 476.90.

Step 2: 4-(4-cyano-2-methoxyphenoxy)-N-(3-(S-methylsulfonimidoyl)phenyl)-6-(trifluoromethyl)pyridazine-3-carboxamide: A solution of 4-(4-cyano-2-methoxyphenoxy)-N-(3-(methylsulfinyl)phenyl)-6-(trifluoromethyl)pyridazine-3-carboxamide (90 mg, 0.19 mmol), NH₄OAc (44 mg, 0.57 mmol) and PhI(OAc)₂ (183 mg, 0.57 mmol) in MeOH (5 mL) was heated at 70° C. for 2 hours. The mixture was concentrated under vacuum. The residue was purified by prep-HPLC (Gemini 5 um C₁₈ column, 150*21.2 mm, eluting with 30% to 75% MeCN/H₂O containing 0.1% formic acid) to obtain 4-(4-cyano-2-methoxyphenoxy)-N-(3-(S-methylsulfonimidoyl)phenyl)-6-(trifluoromethyl)pyridazine-3-carboxamide (34.48 mg, 41.45%) as an off-white solid. ¹H NMR (400 MHz, DMSO-d₆) δ 11.42 (s, 1H), 8.43 (s, 1H), 7.95 (d, J=8.1 Hz, 1H), 7.80 (d, J=1.6 Hz, 1H), 7.72 (d, J=10.8 Hz, 2H), 7.63 (t, J=7.9 Hz, 1H), 7.58 (dd, J=8.3, 1.7 Hz, 1H), 7.53 (d, J=8.3 Hz, 1H), 4.26 (s, 1H), 3.81 (s, 3H), 3.07 (s, 3H). LCMS (ESI) calcd. for C₂₁H₁₇F₃N₅O₄S [M+H]⁺ m/z 492.09, found 491.90.

### Example 35C

### (R)-5-chloro-2-((6-fluoro-2-methylpyridin-3-yl)oxy)-4-methyl-N-(3-(S-methylsulfonimidoyl)phenyl)-6-(trifluoromethyl)nicotinamide

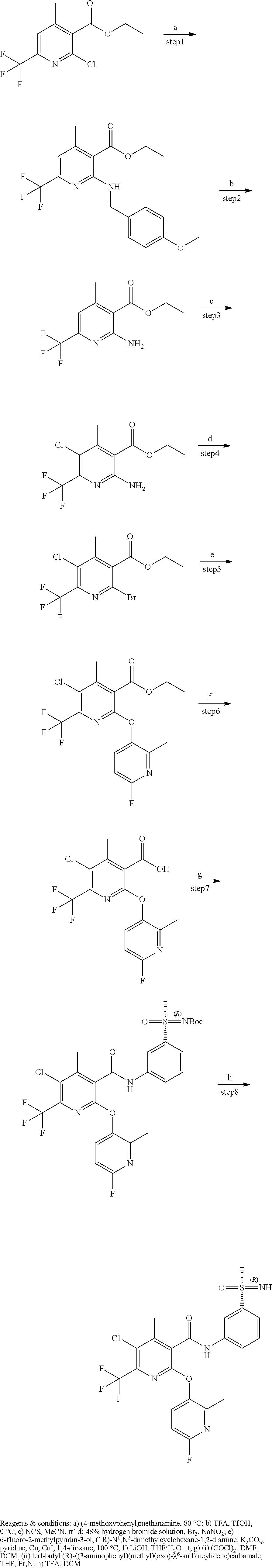

Step 1: ethyl 2-((4-methoxybenzyl)amino)-4-methyl-6-(trifluoromethyl)nicotinate: A e) solution of ethyl 2-chloro-4-methyl-6-(trifluoromethyl)nicotinate (5 g, 0.019 mol) in (4-methoxyphenyl)methanamine (30 mL) was heated at 80° C. for 3 hours. LCMS showed the rection was completed. The mixture was diluted with water and extracted with EtOAc (3×30 mL). The combined organic phases were washed with brine, dried with Na₂SO₄, and concentrated under vacuum. The residue was purified by silica gel column chromatography (eluting with PE/EtOAc, 0 to 10%) to give ethyl 2-((4-methoxybenzyl)amino)-4-methyl-6-(trifluoromethyl)nicotinate (5.5 g, 78.66%) as a yellow oil. LCMS (ESI) calcd. for C₁₈H₂₀F₃N23 [M+H]⁺ m/z 369.14, found 369.10.

Step 2: ethyl 2-amino-4-methyl-6-(trifluoromethyl)nicotinate: To a solution of ethyl 2-((4-methoxybenzyl)amino)-4-methyl-6-(trifluoromethyl)nicotinate (5.5 g, 0.015 mol) in TFA (30 mL) and was added TfOH (3 mL) at 0° C. Then the mixture was stirred at 25° C. for 1 hour. LCMS showed the rection was completed. The mixture was quenched with water and adjusted to pH=8-9 with saturated aqueous NaHCO₃. The mixture extracted with EtOAc (3×30 mL). The combined organic layers were washed with brine, dried with Na₂SO₄, and concentrated under vacuum. The residue was purified by silica gel column chromatography (eluting with EtOAc/PE, 0 to 10%) to give ethyl 2-amino-4-methyl-6-(trifluoromethyl)nicotinate (3.2 g, 86.02%) as a yellow oil. LCMS (ESI) calcd. for C₁₀H₁₂F₃N₂O₂ [M+H]⁺ m/z 249.08, found 249.00.

Step 3: ethyl 2-amino-5-chloro-4-methyl-6-(trifluoromethyl)nicotinate: To a solution of ethyl 2-amino-4-methyl-6-(trifluoromethyl)nicotinate (3.2 g, 0.013 mol) in MeCN (30 mL) and was added NCS (3.5 g, 0.026 mol) at room temperature. Then the mixture was heated at 50° C. for 3 hours under an atmosphere of N2. LCMS showed the rection was completed. The mixture was diluted with water and extracted with EtOAc (3×30 mL). The combined organic layers were washed with brine, dried with Na₂SO₄, and concentrated under vacuum. The residue was purified by silica gel column chromatography (eluting with EtOAc/PE, 0 to 10%) to give ethyl 2-amino-5-chloro-4-methyl-6-(trifluoromethyl)nicotinate (2.8 g, 76.37%) as a yellow solid. LCMS (ESI) calcd. for C₁₀H₁₁ClF₃N₂O₂ [M+H]⁺ m/z 283.04, found 283.00.

Step 4: ethyl 2-bromo-5-chloro-4-methyl-6-(trifluoromethyl)nicotinate: To a solution of ethyl 2-amino-5-chloro-4-methyl-6-(trifluoromethyl)nicotinate (2.8 g, 0.010 mol) in 48% hydrogen bromide solution (5.9 mL) and was added Br₂ (2.4 g, 0.015 mol) and NaNO₂ (1.4 g, 0.020 mol) at 0° C. Then the mixture was stirred at 25° C. for 3 hours. LCMS showed the rection was completed. The mixture was quenched with water and adjusted to pH=8-9 with saturated aqueous NaHCO₃. The mixture extracted with EtOAc (3×30 mL). The combined organic layers were washed with brine, dried with Na₂SO₄, and concentrated under vacuum. The residue was purified by silica gel column chromatography (eluting with EtOAc/PE, 0 to 20%) to give ethyl 2-bromo-5-chloro-4-methyl-6-(trifluoromethyl)nicotinate (1.6 g, 46.37%) as a yellow solid. LCMS (ESI) calcd. for C₁₀H₉BrClF₃NO₂ [M+H]⁺ m/z 345.94, found 345.90.

Step 5: ethyl 5-chloro-2-((6-fluoro-2-methylpyridin-3-yl)oxy)-4-methyl-6-(trifluoromethyl)nicotinate: A solution of ethyl 2-bromo-5-chloro-4-methyl-6-(trifluoromethyl)nicotinate (700 mg, 0.99 mmol), 6-fluoro-2-methylpyridin-3-ol (514 mg, 4.04 mmol), (1R)—N¹,N²-dimethylcyclohexane-1,2-diamine (115 mg, 0.81 mmol), K₂CO₃ (558 mg, 4.04 mmol), pyridine (320 mg, 4.04 mmol), Cu power (26 mg, 0.40 mmol) and CuI (77 mg, 0.40 mmol) in 1,4-dioxane (20 mL) was heated at 100° C. for 16 hours under N2. LCMS showed the rection was completed. The mixture was diluted with water and extracted with EtOAc (3×50 mL). The combined organic layers were washed with brine, dried with Na₂SO₄, and concentrated under vacuum. The residue was purified by flash column chromatography on silica gel (PE/EtOAc=3/1) to give ethyl 5-chloro-2-((6-fluoro-2-methylpyridin-3-yl)oxy)-4-methyl-6-(trifluoromethyl)nicotinate (300 mg, 37.8%) as a yellow oil. LCMS (ESI) calcd. for C₁₆H₁₄ClF₄N₂O₃ [M+H]⁺ m/z 393.06, found 393.05.

Step 6: 5-chloro-2-((6-fluoro-2-methylpyridin-3-yl)oxy)-4-methyl-6-(trifluoromethyl)nicotinic acid: To a solution of ethyl 5-chloro-2-((6-fluoro-2-methylpyridin-3-yl)oxy)-4-methyl-6-(trifluoromethyl)nicotinate (300 mg, 0.76 mmol) in THF/H₂O (1/1, 10 mL) was added LiOH (183 mg, 7.64 mmol) at room temperature. The mixture was stirred at room temperature for 16 hours. After the reaction was completed, the mixture was concentrated to remove most THE. The aqueous phase was adjusted to pH=3-4 with 1N HCl then extracted with EtOAc (30 mL×3). The combined organic layers were washed with brine, dried with Na₂SO₄, concentrated under reduced pressure to give 5-chloro-2-((6-fluoro-2-methylpyridin-3-yl)oxy)-4-methyl-6-(trifluoromethyl)nicotinic acid (220 mg, 79.0%) as a yellow solid. LCMS (ESI) calcd. for C₁₄H₁₀ClF₄N₂O₃ [M+H]⁺ m/z 365.03, found 364.95.

Step 7: tert-butyl (R)-((3-(5-chloro-2-((6-fluoro-2-methylpyridin-3-yl)oxy)-4-methyl-6-(trifluoromethyl)nicotinamido)phenyl)(methyl)(oxo)-λ⁶-sulfaneylidene)carbamate: To a solution of 5-chloro-2-((6-fluoro-2-methylpyridin-3-yl)oxy)-4-methyl-6-(trifluoromethyl)nicotinic acid (220 mg, 0.60 mmol) in DCM (5 mL) was added oxalyl chloride (766 mg, 6.03 mmol) and DMF (20 μL). The mixture was stirred at room temperature for 0.5 hour. Then the mixture was concentrated under vacuum. The residue was dissolved in THF (5 mL) and added to a solution of tert-butyl (R)-((3-aminophenyl)(methyl)(oxo)-l6-sulfaneylidene)carbamate (179 mg, 0.66 mmol) and Et₃N (305 mg, 3.02 mmol) in THF (5 mL). The mixture was stirred at room temperature for 1 hour. After the reaction was completed, the mixture was concentrated under vacuum. The residue was purified by flash column chromatography on silica gel (PE/EtOAc=2/1) to give tert-butyl (R)-((3-(5-chloro-2-((6-fluoro-2-methylpyridin-3-yl)oxy)-4-methyl-6-(trifluoromethyl)nicotinamido)phenyl)(methyl)(oxo)-λ⁶-sulfaneylidene)carbamate (120 mg, 32.2%) as a yellow oil. LCMS (ESI) calcd. for C₂₆H₂₆ClF₄N₄O₅S [M+H]⁺ m/z 617.12, found 617.05.

Step 8: (R)-5-chloro-2-((6-fluoro-2-methylpyridin-3-yl)oxy)-4-methyl-N-(3-(S-methylsulfonimidoyl)phenyl)-6-(trifluoromethyl)nicotinamide. A solution of tert-butyl (R)-((3-(5-chloro-2-((6-fluoro-2-methylpyridin-3-yl)oxy)-4-methyl-6-(trifluoromethyl)nicotinamido)phenyl)(methyl)(oxo)-λ⁶-sulfaneylidene)carbamate (120 mg, 0.19 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (30 mL×3). The combined organic phases were washed with brine, dried over sodium sulfate, concentrated under vacuum. The residue was purified by prep-HPLC (Gemini 5 um C₁₈ column, 150*21.2 mm, eluting with 40% to 95% MeCN/H₂O containing 0.05% NH₃) to provide (R)-5-chloro-2-((6-fluoro-2-methylpyridin-3-yl)oxy)-4-methyl-N-(3-(S-methylsulfonimidoyl)phenyl)-6-(trifluoromethyl)nicotinamide (32.10 mg, 31.9%) as a white solid. ¹H NMR (400 MHz, DMSO-d₆) δ 11.21 (s, 1H), 8.38 (s, 1H), 7.91-7.84 (m, 2H), 7.72 (d, J=7.8 Hz, 1H), 7.63 (t, J=7.9 Hz, 1H), 7.12 (dd, J=8.7, 3.4 Hz, 1H), 4.27 (s, 1H), 3.08 (s, 3H), 2.52 (s, 3H), 2.26 (s, 3H). LCMS (ESI) calcd. for C₂₁H₁₈ClF₄N₄O₃S [M+H]⁺ m/z 517.06, found 517.05.

### Example 36C

### (R)-5-fluoro-3-((6-fluoro-2-methylpyridin-3-yl)oxy)-N-(3-(S-methylsulfonimidoyl)phenyl)-2-(trifluoromethyl)isonicotinamide

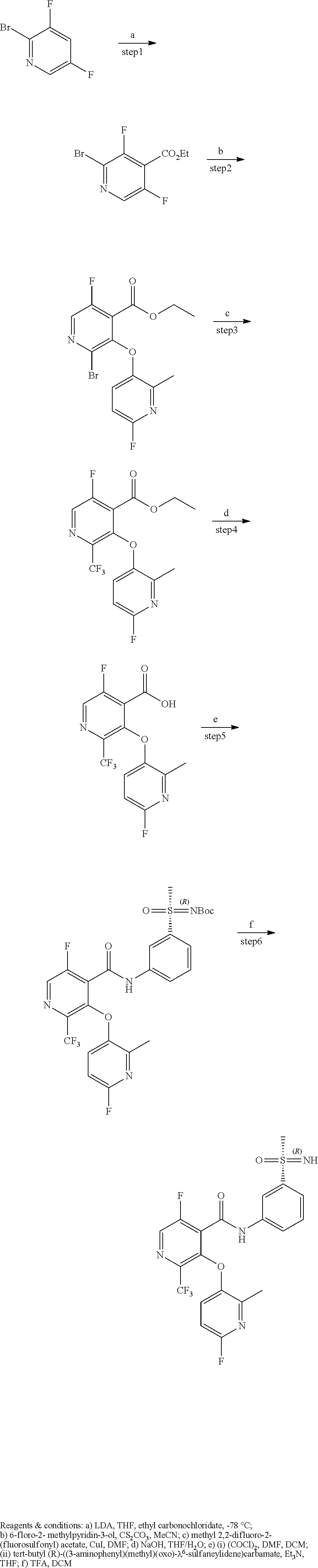

Step 1: ethyl 2-bromo-3,5-difluoroisonicotinate: To a solution of 2-bromo-3,5-difluoropyridine 1 (1.2 g, 10.31 mmol) in THF (20 mL) at −78° C. under an atmosphere of N2 was added a solution of LDA (7.7 mL, 2M in THF). The mixture was stirred at −78° C. for 30 mins. Then a solution of ethyl carbonochloridate (2.24 g, 20.62 mmol) was added to the solution dropwise. The reaction mixture was stirred at 25° C. for 1 hour. After the reaction was completed, the mixture was quenched with saturated NH₄Cl (20 mL) and extracted with EtOAc (30 mL×3). The combined organic layers were dried over sodium sulfate, concentrated under vacuum. The residue was purified by flash column chromatography on silica gel (PE/EtOAc=10/1) to provide ethyl 2-bromo-3,5-difluoroisonicotinate (1.2 g, 43.8%) as colorless oil. LCMS (ESI) calcd. for C₈H₇BrF₂NO₂ [M+H]⁺ m/z 265.96, found 265.95.

Step 2: ethyl 2-bromo-5-fluoro-3-((6-fluoro-2-methylpyridin-3-yl)oxy)isonicotinate: To a solution of ethyl 2-bromo-3,5-difluoroisonicotinate (1.0 g, 3.76 mmol) and 6-fluoro-2-methylpyridin-3-ol (478 mg, 3.76 mmol) in MeCN (20 mL) was added Cs₂CO₃ (1.84 g, 5.64 mmol), the reaction mixture was stirred at room temperature for 12 hours. After the reaction was completed, the mixture was cooled to room temperature. The mixture was diluted with water and extracted with EtOAc (30 mL×3). The combined organic layers were dried over sodium sulfate, concentrated under vacuum. The residue was purified by flash column chromatography on silica gel (PE/EtOAc=10/1) to give ethyl 2-bromo-5-fluoro-3-((6-fluoro-2-methylpyridin-3-yl)oxy)isonicotinate (700 mg, 49.9) as a red oil. LCMS (ESI) calcd. for C₁₄H₁₂BrF₂N₂O₃ [M+H]⁺ m/z 373.00, found 372.95 Step 3: ethyl 5-fluoro-3-((6-fluoro-2-methylpyridin-3-yl)oxy)-2-(trifluoromethyl)isonicotinate: To a solution of ethyl 2-bromo-5-fluoro-3-((6-fluoro-2-methylpyridin-3-yl)oxy)isonicotinate (400 mg, 1.07 mmol) and CuI (408 mg, 2.14 mmol) in DMF (10 mL) was added methyl 2,2-difluoro-2-(fluorosulfonyl) acetate (1.03 g, 5.36 mmol) dropwise at room temperature under an atmosphere of N2. The mixture was heated to 120° C. for 6 hours. After the reaction was completed, the resulting solution was diluted with water (50 mL) and extracted with EtOAc (30 mL×3). The combined organic layers were washed with brine, dried over sodium sulfate, concentrated under vacuum to give the crude. The residue was purified by flash column chromatography on silica gel (PE/EtOAc=10/1 to 3/1) to give ethyl 5-fluoro-3-((6-fluoro-2-methylpyridin-3-yl)oxy)-2-(trifluoromethyl)isonicotinate (250 mg, 64.2%). LCMS (ESI) calcd. for C₁₅H₁₂F₅N₂O₃ [M+H]⁺ m/z 363.08, found 363.00.

Step 4: 5-fluoro-3-((6-fluoro-2-methylpyridin-3-yl)oxy)-2-(trifluoromethyl)isonicotinic acid: To a solution of ethyl 5-fluoro-3-((6-fluoro-2-methylpyridin-3-yl)oxy)-2-(trifluoromethyl)isonicotinate (250 mg, 0.69 mmol) in THF/H₂O (1/1, 5 mL) was added NaOH (138 mg, 3.45 mmol) at room temperature. The reaction mixture was stirred at room temperature for 18 hours. After the reaction was completed, the mixture was concentrated. The residue was adjusted to pH=3-4 with aqueous HCl (1M). Then the solution was extracted with EtOAc (80 mL×3). The combined organic layers were washed with brine, dried over sodium sulfate, concentrated under vacuum to provide 5-fluoro-3-((6-fluoro-2-methylpyridin-3-yl)oxy)-2-(trifluoromethyl)isonicotinic acid (100 mg, 43.4%) as a white solid. LCMS (ESI) calcd. for C₁₃H₈F₅N₂O₃ [M+H]⁺ m/z 335.05, found 335.05.

Step 5: tert-butyl (R)-((3-(5-fluoro-3-((6-fluoro-2-methylpyridin-3-yl)oxy)-2-(trifluoromethyl)isonicotinamido)phenyl)(methyl)(oxo)-λ⁶-sulfaneylidene)carbamate: To a solution of 5-fluoro-3-((6-fluoro-2-methylpyridin-3-yl)oxy)-2-(trifluoromethyl)isonicotinic acid (100 mg, 0.30 mmol) in DCM (3 mL) was added oxalyl chloride (380 mg, 2.30 mmol) and DMF (20 L). The mixture was stirred at room temperature for 0.5 hour. Then the mixture was concentrated under vacuum. The residue was dissolved in THF (5 mL) and added to a solution of tert-butyl (R)-((3-aminophenyl)(methyl)(oxo)-λ⁶-sulfaneylidene)carbamate (97 mg, 0.36 mmol) and TEA (151 mg, 1.50 mmol) in THF (5 mL). The mixture was stirred at room temperature for 1 hour. After the reaction was completed, the mixture was concentrated under vacuum. The residue was purified by flash column chromatography on silica gel (PE/EtOAc=2/1) to give tert-butyl (R)-((3-(5-fluoro-3-((6-fluoro-2-methylpyridin-3-yl)oxy)-2-(trifluoromethyl)isonicotinamido)phenyl)(methyl)(oxo)-l6-sulfaneylidene)carbamate 6 (90 mg, 51.3% yield) as a yellow oil. LCMS (ESI) calcd. for C₂₅H₂₄F₅N₄O₅S [M+H]⁺ m/z 587.14, found 587.00.

Step 6: (R)-5-fluoro-3-((6-fluoro-2-methylpyridin-3-yl)oxy)-N-(3-(S-methylsulfonimidoyl)phenyl)-2-(trifluoromethyl)isonicotinamide: A solution of tert-butyl (R)-((3-(5-fluoro-3-((6-fluoro-2-methylpyridin-3-yl)oxy)-2-(trifluoromethyl)isonicotinamido)phenyl)(methyl)(oxo)-λ⁶-sulfaneylidene)carbamate (90 mg, 0.16 mmol) in DCM (5 mL) was added TFA (1 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 40% to 90% MeCN/H₂O containing 0.05% NH₃) to provide (R)-5-fluoro-3-((6-fluoro-2-methylpyridin-3-yl)oxy)-N-(3-(S-methylsulfonimidoyl)phenyl)-2-(trifluoromethyl)isonicotinamide (75.4 mg, 90.9%) as a white solid. ¹H NMR (400 MHz, DMSO-d₆) δ 11.23 (s, 1H), 8.91 (s, 1H), 7.90 (s, 1H), 7.66 (s, 1H), 7.57-7.47 (m, 3H), 6.90 (dd, J=8.8, 3.3 Hz, 1H), 4.20 (s, 1H), 3.03 (s, 3H), 2.31 (s, 3H). LCMS (ESI) calcd. for C₂₀H₁₆F₅N₄O₃S [M+H]⁺ m/z 487.09, found 487.00.

### Example 37C

### (R)-2-cyclopropyl-5-fluoro-3-((6-fluoro-2-methylpyridin-3-yl)oxy)-N-(3-(S-methylsulfonimidoyl)phenyl)isonicotinamide

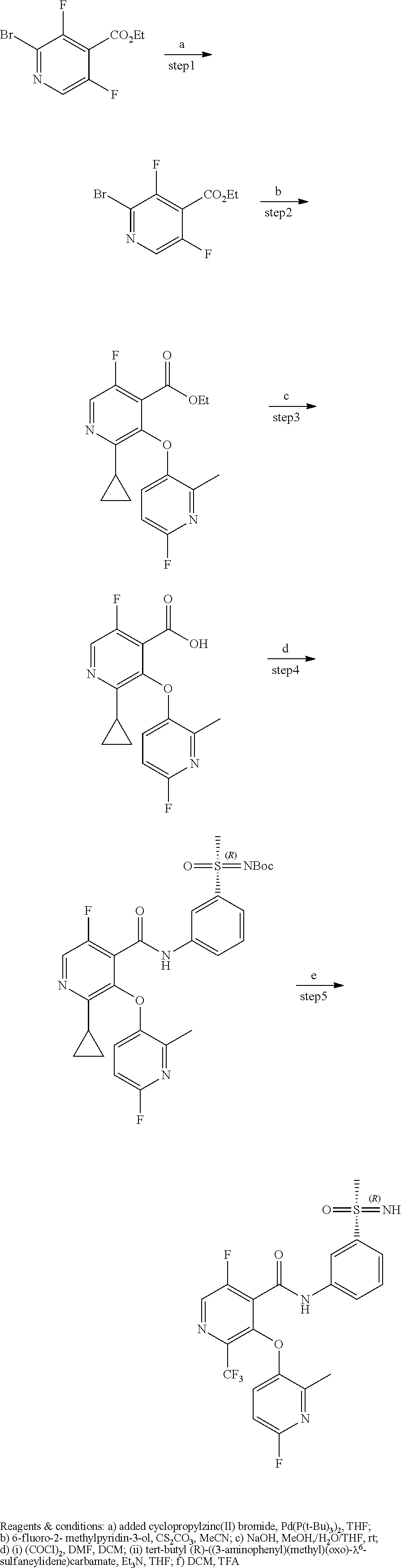

Step 1. ethyl 2-cyclopropyl-3,5-difluoroisonicotinate: To a solution of ethyl 2-bromo-3,5-difluoroisonicotinate (500 mg, 1.88 mmol) and Pd(P(t-Bu)₃)₂ (193 mg, 0.38 mmol) in THF (10 mL) was added cyclopropylzinc(II) bromide (4 mL, 0.5M in THF) dropwise at room temperature under an atmosphere of N2. The mixture was stirred at room temperature for 6 hours. After the reaction was completed, the resulting solution was diluted with water (30 mL) and extracted with EtOAc (10 mL×3). The combined organic layers were washed with brine, dried over sodium sulfate, concentrated under vacuum. The residue was purified by flash column chromatography on silica gel (PE/EtOAc=10/1 to 3/1) to give ethyl 2-cyclopropyl-3,5-difluoroisonicotinate (300 mg, 70.3%). LCMS (ESI) calcd. for C₁₁H₁₂F₂NO₂ [M+H]⁺ m/z 228.09, found 228.10.

Step 2: ethyl 2-cyclopropyl-5-fluoro-3-((6-fluoro-2-methylpyridin-3-yl)oxy)isonicotinate: To a solution of ethyl 2-cyclopropyl-3,5-difluoroisonicotinate (300 mg, 1.13 mmol) and 6-fluoro-2-methylpyridin-3-ol (143 mg, 1.13 mmol) in MeCN (10 mL) was added Cs₂CO₃ (404 mg, 1.24 mmol), the reaction mixture was stirred at room temperature for 12 hours. After the reaction was completed, the mixture was cooled to room temperature. The mixture was diluted with water and extracted with EtOAc (30 mL×3). The combined organic layers were dried over sodium sulfate, concentrated under vacuum. The residue was purified by flash column chromatography on silica gel (PE/EtOAc=10/1) to give ethyl 2-cyclopropyl-5-fluoro-3-((6-fluoro-2-methylpyridin-3-yl)oxy)isonicotinate (200 mg, 47.5%) as a red oil. LCMS (ESI) calcd. for C₁₇H₁₇F₂N₂O₃ [M+H]f m/z 335.12, found 335.05.

Step 3: 2-cyclopropyl-5-fluoro-3-((6-fluoro-2-methylpyridin-3-yl)oxy)isonicotinic acid: To a solution of ethyl 2-cyclopropyl-5-fluoro-3-((6-fluoro-2-methylpyridin-3-yl)oxy)isonicotinate (200 mg, 0.60 mmol) in MeOH/THF/H₂O (1/1/1, 6 mL) was added NaOH (120 mg, 2.99 mmol) at room temperature. The reaction mixture was stirred at room temperature for 18 hours. After the reaction was completed, the mixture was concentrated. The residue was adjusted to pH=3-4 with aqueous HCl (1M). Then the solution was extracted with EtOAc (80 mL×3). The combined organic layers were washed with brine, dried over sodium sulfate, concentrated under vacuum to provide 2-cyclopropyl-5-fluoro-3-((6-fluoro-2-methylpyridin-3-yl)oxy)isonicotinic acid (100 mg, 54.6%) as a white solid. LCMS (ESI) calcd. for C₁₅H₁₃F₂N₂O₃ [M+H]⁻ m/z 307.09, found 307.05.

Step 4: tert-butyl (R)-((3-(2-cyclopropyl-5-fluoro-3-((6-fluoro-2-methylpyridin-3-yl)oxy)isonicotinamido)phenyl)(methyl)(oxo)-λ⁶-sulfaneylidene)carbamate: To a solution of 2-cyclopropyl-5-fluoro-3-((6-fluoro-2-methylpyridin-3-yl)oxy)isonicotinic acid (100 mg, 0.33 mmol) in DCM (3 mL) was added oxalyl chloride (414 mg, 3.27 mmol) and DMF (20 μL). The mixture was stirred at room temperature for 0.5 hour. Then the mixture was concentrated under vacuum. The residue was dissolved in THE (5 mL) and was added to a solution of tert-butyl (R)-((3-aminophenyl)(methyl)(oxo)-λ⁶-sulfaneylidene)carbamate (106 mg, 0.39 mmol) and Et₃N (165 mg, 1.63 mmol) in THF (5 mL). The mixture was stirred at room temperature for 1 hour. After the reaction was completed, the mixture was concentrated under vacuum. The residue was purified by flash column chromatography on silica gel (PE/EtOAc=2/1) to give tert-butyl (R)-((3-(2-cyclopropyl-5-fluoro-3-((6-fluoro-2-methylpyridin-3-yl)oxy)isonicotinamido)phenyl)(methyl)(oxo)-λ⁶-sulfaneylidene)carbamate (65 mg, 35.7%) as a yellow oil. LCMS (ESI) calcd. for C₂₇H₂₉F₂N₄O₅S [M+H]⁺ m/z 559.18, found 559.10.

Step 5: (R)-2-cyclopropyl-5-fluoro-3-((6-fluoro-2-methylpyridin-3-yl)oxy)-N-(3-(S-methylsulfonimidoyl)phenyl)isonicotinamide: A solution of tert-butyl (R)-((3-(2-cyclopropyl-5-fluoro-3-((6-fluoro-2-methylpyridin-3-yl)oxy)isonicotinamido)phenyl)(methyl)(oxo)-λ⁶-sulfaneylidene)carbamate (65 mg, 0.12 mmol) in DCM (5 mL) was added TFA (1 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic phases were washed with brine, dried over sodium sulfate, concentrated under vacuum. The residue was purified by prep-HPLC (Gemini 5 um C₁₈ column, 150*21.2 mm, eluting with 40% to 90% MeCN/H₂O containing 0.05% NH₃) to provide (R)-2-cyclopropyl-5-fluoro-3-((6-fluoro-2-methylpyridin-3-yl)oxy)-N-(3-(S-methylsulfonimidoyl)phenyl)isonicotinamide (41.3 mg, 77.4%) as a white solid. ¹H NMR (400 MHz, DMSO-d₆) δ 11.17 (s, 1H), 8.60 (s, 1H), 8.09 (s, 1H), 7.66 (s, 2H), 7.55 (t, J=7.8 Hz, 1H), 7.21 (dd, J=8.7, 6.5 Hz, 1H), 6.92 (dd, J=8.8, 3.4 Hz, 1H), 4.21 (s, 1H), 3.03 (s, 3H), 2.37 (s, 3H), 2.15-2.07 (m, 1H), 1.01-0.88 (m, 4H). LCMS (ESI) calcd. for C₂₂H₂₁F₂N₄O₃S [M+H]⁺ m/z 459.13, found 459.05.

### Example 38C

### 4-(4-cyano-2-methoxyphenoxy)-N-(3-(methylsulfonyl)phenyl)-6-(trifluoromethyl)pyridazine-3-carboxamide

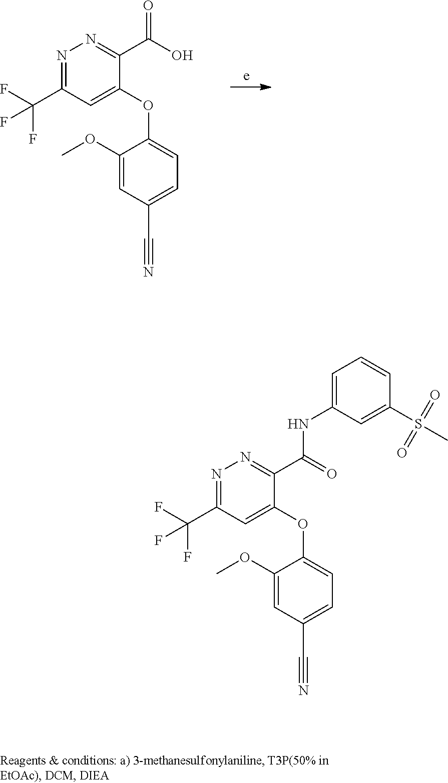 [SMILES: COc1cc(C#N)ccc1Oc1cc(C(F)(F)F)nnc1C(=O)O ; COc1cc(C#N)ccc1Oc1cc(C(F)(F)F)nnc1C(=O)Nc1cccc(S(C)(=O)=O)c1]

To a solution of 4-(4-cyano-2-methoxyphenoxy)-6-(trifluoromethyl) pyridazine-3-carboxylic acid (50 mg, 0.15 mmol), 3-methanesulfonylaniline (25 mg, 0.15 mmol) and T3P (224 mg, 50% in EtOAc) in DCM (5 mL) was added DIEA (114 mg, 0.88 mmol). The mixture was stirred at room temperate for 2 hours. The mixture was diluted with water (10 mL) and extracted with EtOAc (10 mL×3). The combine organic phases were washed with brine, dried over sodium sulfate, concentrated under vacuum. The residue was first purified by flash column chromatography on silica gel (eluting with EtOAc/PE, 0% to 90%) to afford 4-(4-cyano-2-methoxyphenoxy)-N-(3-(methylsulfonyl) phenyl)-6-(trifluoromethyl) pyridazine-3-carboxamide (29.90 mg, 41.20%) as an off-white solid. ¹H NMR (400 MHz, DMSO-d₆) δ 11.49 (s, 1H), 8.44 (q, J=1.7 Hz, 1H), 8.07-7.94 (m, 1H), 7.81 (d, J=1.7 Hz, 1H), 7.76-7.67 (m, 3H), 7.58 (dd, J=8.3, 1.7 Hz, 1H), 7.53 (d, J=8.3 Hz, 1H), 3.88-3.73 (m, 3H), 3.26-3.17 (m, 3H). LCMS (ESI) calcd. for C₂₁H₁₆F₃N₄O₅S [M+H]⁺ m/z 493.07, found 492.95.

### Example 39C

### 4-(4-cyano-2-methylphenoxy)-N-(3-(S-methylsulfonimidoyl)phenyl)-6-(trifluoromethyl)pyridazine-3-carboxamide

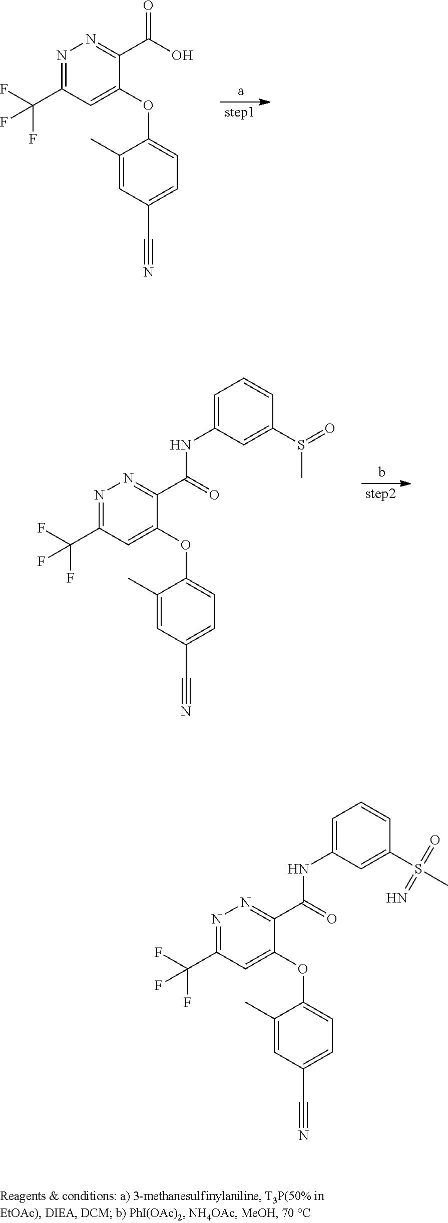 [SMILES: Cc1cc(C#N)ccc1Oc1cc(C(F)(F)F)nnc1C(=O)O ; Cc1cc(C#N)ccc1Oc1cc(C(F)(F)F)nnc1C(=O)Nc1cccc(S(C)=O)c1 ; Cc1cc(C#N)ccc1Oc1cc(C(F)(F)F)nnc1C(=O)Nc1cccc(S(C)(=N)=O)c1]

Step 1: 4-(4-cyano-2-methylphenoxy)-N-(3-(methylsulfinyl)phenyl)-6-(trifluoromethyl)pyridazine-3-carboxamide: To a solution of 4-(4-cyano-2-methylphenoxy)-6-(trifluoromethyl)pyridazine-3-carboxylic acid (240 mg, 0.74 mmol), 3-methanesulfinylaniline (115 mg, 0.74 mmol) and T3P (945 mg, 50% in EtOAc) in DCM (5 mL) was added DIEA (574 mg, 4.45 mmol). The mixture was stirred at room temperate for 2 hours. Then the mixture was diluted with water (10 mL) and extracted with EtOAc (10 mL×3). The combine organic phases were washed with brine, dried over sodium sulfate, concentrated under vacuum. The residue was purified by flash column chromatography on silica gel (eluting with EtOAc/PE, 0% to 90%) to afford 4-(4-cyano-2-methylphenoxy)-N-(3-methanesulfinylphenyl)-6-(trifluoromethyl)pyridazine-3-carboxamide (160 mg, 46.80%) as an off-white solid. LCMS (ESI) calcd. for C₂₁H₁₆F₃N₄O₃S [M+H]⁺ m/z 461.09, found 461.10.

Step 2: 4-(4-cyano-2-methylphenoxy)-N-(3-(S-methylsulfonimidoyl)phenyl)-6-(trifluoromethyl)pyridazine-3-carboxami: A solution of 4-(4-cyano-2-methylphenoxy)-N-(3-(methylsulfinyl)phenyl)-6-(trifluoromethyl)pyridazine-3-carboxamide (160 mg, 0.35 mmol), NH₄OAc (80 mg, 1.04 mmol) and PhI(OAc)₂ (336 mg, 1.23 mmol) in MeOH (5 mL) was heated at 70° C. for 2 hours. The mixture was concentrated under vacuum. The residue was purified by prep-HPLC (Gemini 5 um C₁₈ column, 150*21.2 mm, eluting with 30% to 75% MeCN/H₂O containing 0.1% formic acid) to afford 4-(4-cyano-2-methylphenoxy)-N-(3-(S-methylsulfonimidoyl)phenyl)-6-(trifluoromethyl)pyridazine-3-carboxamide (26.3 mg, 15.91%) as an off-white solid. ¹H NMR (400 MHz, DMSO-d₆) δ 11.42 (s, 1H), 8.40 (d, J=1.8 Hz, 1H), 7.97-7.89 (m, 2H), 7.83 (dd, J=8.5, 1.9 Hz, 1H), 7.77-7.70 (m, 2H), 7.63 (t, J=7.9 Hz, 1H), 7.47 (d, J=8.5 Hz, 1H), 4.26 (s, 1H), 3.07 (s, 3H), 2.23 (s, 3H). LCMS (ESI) calcd. for C₂₁H₁₇F₃N₅O₃S [M+H]⁺ m/z 476.10, found 476.05.

### Example 40C

### ((3-(2-((6-fluoro-2-methylpyridin-3-yl)oxy)-4-methyl-5-(trifluoromethyl)nicotinamido)phenyl)(methyl)-l6-sulfanediylidene)diamine

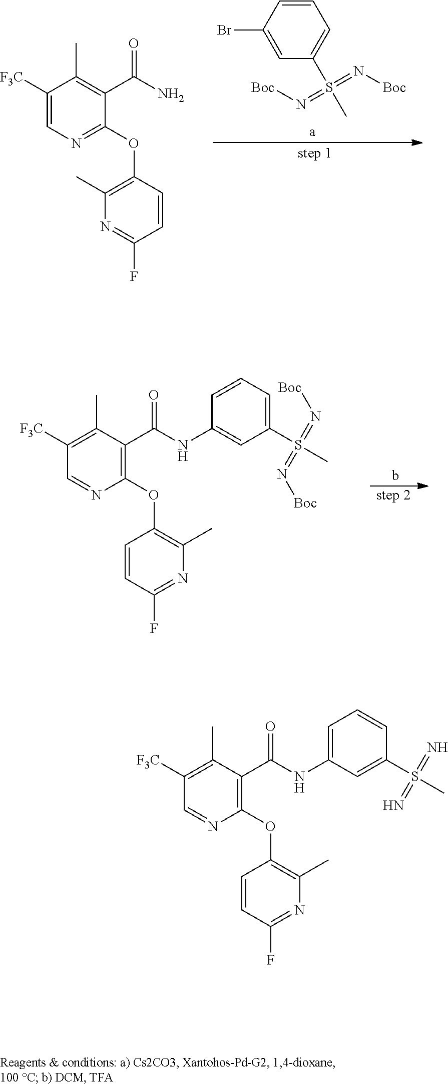 [SMILES: Cc1nc(F)ccc1Oc1ncc(C(F)(F)F)c(C)c1C(N)=O ; CC(C)(C)OC(=O)N=S(C)(=NC(=O)OC(C)(C)C)c1cccc(Br)c1 ; Cc1nc(F)ccc1Oc1ncc(C(F)(F)F)c(C)c1C(=O)Nc1cccc(S(C)(=NC(=O)OC(C)(C)C)=NC(=O)OC(C)(C)C)c1 ; Cc1nc(F)ccc1Oc1ncc(C(F)(F)F)c(C)c1C(=O)Nc1cccc(S(C)(=N)=N)c1]

Step 1: A mixture of 2-((6-fluoro-2-methylpyridin-3-yl)oxy)-4-methyl-5-(trifluoromethyl)nicotinamide (300 mg, 0.91 mmol), di-tert-butyl ((3-bromophenyl)(methyl)-16-sulfanediylidene)dicarbamate (470 mg, 1.09 mmol), Cs₂CO₃ (890 mg, 2.73 mmol) and Xantphos-Pd-G2 (80 mg, 0.09 mmol) in dioxane (10 mL) was heated to 100° C. and stirred for 16 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to provide di-tert-butyl ((3-(2-((6-fluoro-2-methylpyridin-3-yl)oxy)-4-methyl-5-(trifluoromethyl)nicotinamido)phenyl)(methyl)-16-sulfanediylidene)dicarbamate (200 mg, 32.2% yield) as a yellow solid. LCMS (ESI) calcd. for C₃₁H₃₆F₄N₅O₆S [M+H]⁺ m/z 682.23, found 682.05.

Step 2: To a solution of di-tert-butyl ((3-(2-((6-fluoro-2-methylpyridin-3-yl)oxy)-4-methyl-5-(trifluoromethyl)nicotinamido)phenyl)(methyl)-l6-sulfanediylidene)dicarbamate (200 mg, 0.29 mmol) in DCM (5 mL) was added TFA (0.5 mL). The mixture was stirred at room temperature for 2 hours. After the reaction was completed, the mixture was concentrated. The residue was adjusted to pH=8-9 with saturated aqueous NaHCO₃. Then the aqueous solution was extracted with DCM (15 mL×3). The combined organic layers were washed with brine, dried over sodium sulfate, concentrated under vacuum. The residue was purified by prep-HPLC (Gemini um C₁₈ column, 150*21.2 mm, eluting with 50% to 95% MeCN/H₂O containing 0.05% NH₃) to provide ((3-(2-((6-fluoro-2-methylpyridin-3-yl)oxy)-4-methyl-5-(trifluoromethyl)nicotinamido) phenyl)(methyl)-l6-sulfanediylidene)diamine (54.3 mg, 38.30% yield) as a white solid. ¹H NMR (400 MHz, DMSO-d₆, ppm) δ 11.16 (s, 1H), 8.54 (s, 1H), 8.49 (t, J=1.8 Hz, 1H), 7.91-7.78 (m, 3H), 7.58 (t, J=8.0 Hz, 1H), 7.11 (dd, J=8.7, 3.4 Hz, 1H), 3.06-2.92 (m, 5H), 2.49-2.48 (m, 3H), 2.25 (s, 3H). LCMS (ESI) calcd. for C₂₁H₂₀F₄N₅O₂S [M+H]⁺ m/z 482.13, found 482.05.

### Example 41C

### N-(3-(3-amino-3-ethylpentylsulfonimidoyl)phenyl)-2-((6-fluoro-2-methylpyridin-3-yl)oxy)-4-methyl-5-(trifluoromethyl)nicotinamide

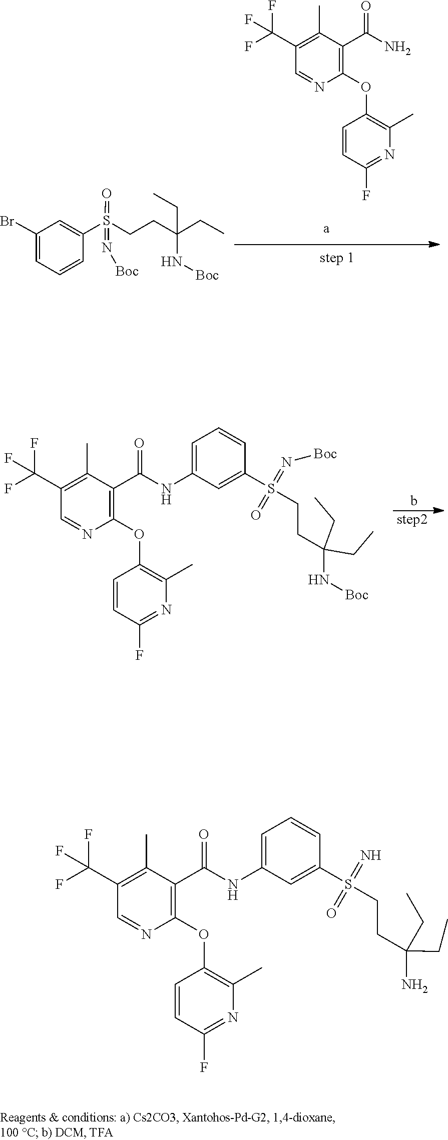 [SMILES: CCC(CC)(CCS(=O)(=NC(=O)OC(C)(C)C)c1cccc(Br)c1)NC(=O)OC(C)(C)C ; Cc1nc(F)ccc1Oc1ncc(C(F)(F)F)c(C)c1C(N)=O ; CCC(CC)(CCS(=O)(=NC(=O)OC(C)(C)C)c1cccc(NC(=O)c2c(Oc3ccc(F)nc3C)ncc(C(F)(F)F)c2C)c1)NC(=O)OC(C)(C)C ; CCC(N)(CC)CCS(=N)(=O)c1cccc(NC(=O)c2c(Oc3ccc(F)nc3C)ncc(C(F)(F)F)c2C)c1]

Step: tert-butyl ((3-((tert-butoxycarbonyl)amino)-3-ethylpentyl)(3-(2-((6-fluoro-2-methylpyridin-3-yl)oxy)-4-methyl-5-(trifluoromethyl)nicotinamido)phenyl)(oxo)-16-sulfaneylidene)carbamate. A mixture of tert-butyl ((3-bromophenyl)(3-((tert-butoxycarbonyl)amino)-3-ethylpentyl)(oxo)-l6-sulfaneylidene)carbamate (389.6 mg, 0.73 mmol), 2-((6-fluoro-2-methylpyridin-3-yl)oxy)-4-methyl-5-(trifluoromethyl)nicotinamide (See Example 40C, 200 mg, 0.61 mmol), Cs₂CO₃ (593.71 mg, 1.82 mmol) and Xantphos-Pd-G2 (26.99 mg, 0.03 mmol) in 1,4-dioxane (10 mL) under N2 was stirred at 100° C. for 2 hours. After the reaction was completed, the resulting solution was diluted with water (50 mL) and extracted with EtOAc (25 mL×3). The combined organic phases were washed with brine, dried over Na₂SO₄, concentrated under vacuum. The residue was purified by flash column chromatography on silica gel (PE/EtOAc=1/1) to provide tert-butyl ((3-((tert-butoxycarbonyl)amino)-3-ethylpentyl)(3-(2-((6-fluoro-2-methylpyridin-3-yl)oxy)-4-methyl-5-(trifluoromethyl)nicotinamido)phenyl)(oxo)-16-sulfaneylidene)carbamate (220 mg, 46.26% yield) as a yellow solid. LCMS (ESI) calcd. for C₃₇H₄₈F₄N₅O₇S [M+H]⁺ m/z 782.32, found 782.1.

Step 2: N-(3-(3-amino-3-ethylpentylsulfonimidoyl)phenyl)-2-((6-fluoro-2-methylpyridin-3-yl)oxy)-4-methyl-5-(trifluoromethyl)nicotinamide: A mixture of tert-butyl ((3-((tert-butoxycarbonyl)amino)-3-ethylpentyl)(3-(2-((6-fluoro-2-methylpyridin-3-yl)oxy)-4-methyl-5-(trifluoromethyl)nicotinamido)phenyl)(oxo)-l6-sulfaneylidene)carbamate (220 mg, 0.28 mmol) in DCM (10 mL) was added TFA (2 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (25 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 85% MeCN/H₂O containing 0.05% NH₃) to obtain N-(3-(3-amino-3-ethylpentylsulfonimidoyl)phenyl)-2-((6-fluoro-2-methylpyridin-3-yl)oxy)-4-methyl-5-(trifluoromethyl)nicotinamide (41 mg, 36.4% yield) as a white solid. ¹H NMR (400 MHz, CD₃OD, ppm) δ 8.45 (t, J=1.9 Hz, 1H), 8.42 (s, 1H), 7.95 (dd, J=8.1, 1.1 Hz, 1H), 7.81-7.71 (m, 2H), 7.65 (t, J=8.0 Hz, 1H), 6.96 (dd, J=8.7, 3.1 Hz, 1H), 3.25 (dd, J=9.4, 7.7 Hz, 2H), 2.58 (s, 3H), 2.29 (s, 3H), 1.81-1.59 (m, 2H), 1.34 (q, J=7.1 Hz, 4H), 0.76 (t, J=7.5 Hz, 6H). LCMS (ESI) calcd. for C₂₇H₃₂F₄N₅O₃S [M+H]⁺ m/z 582.22, found 582.2.

### Example 42C

### 2-((6-fluoro-2-methylpyridin-3-yl)oxy)-N-(3-(3-hydroxy-3-methylbutylsulfonimidoyl)phenyl)-4-methyl-5-(trifluoromethyl)nicotinamide

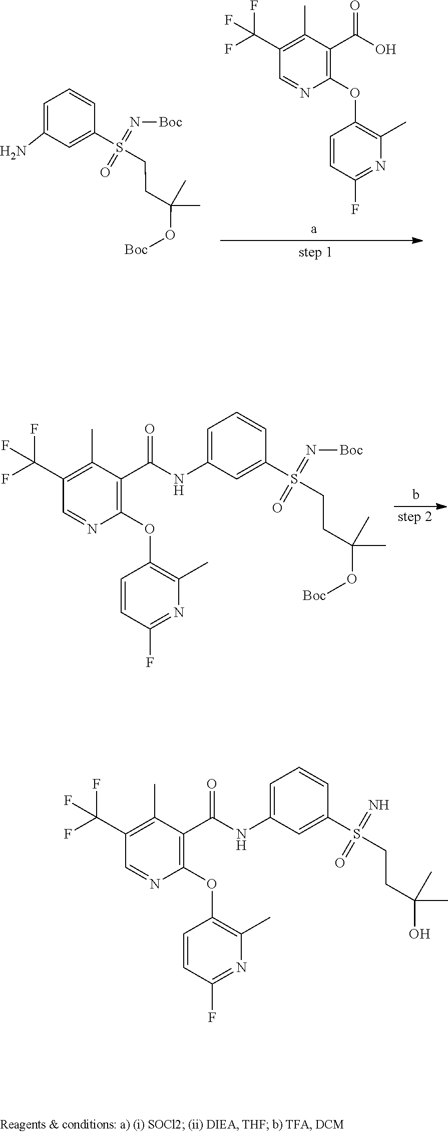 [SMILES: CC(C)(C)OC(=O)N=S(=O)(CCC(C)(C)OC(=O)OC(C)(C)C)c1cccc(N)c1 ; Cc1nc(F)ccc1Oc1ncc(C(F)(F)F)c(C)c1C(=O)O ; Cc1nc(F)ccc1Oc1ncc(C(F)(F)F)c(C)c1C(=O)Nc1cccc(S(=O)(CCC(C)(C)OC(=O)OC(C)(C)C)=NC(=O)OC(C)(C)C)c1 ; Cc1nc(F)ccc1Oc1ncc(C(F)(F)F)c(C)c1C(=O)Nc1cccc(S(=N)(=O)CCC(C)(C)O)c1]

Step 1. tert-butyl ((3-((tert-butoxycarbonyl)oxy)-3-methylbutyl)(3-(2-((6-fluoro-2-methylpyridin-3-yl)oxy)-4-methyl-5-(trifluoromethyl)nicotinamido)phenyl)(oxo)-16-sulfaneylidene)carbamate: A solution of 2-((6-fluoro-2-methylpyridin-3-yl)oxy)-4-methyl-5-(trifluoromethyl)nicotinic acid (223.48 mg, 0.68 mmol) in SOCl₂ (5 mL) was heated to 80° C. for 0.5 hours. The solution was concentrated under vacuum to provide the chloride intermediate. Then the chloride intermediate dissolved in THE (5 mL) was added to a solution of tert-butyl ((3-aminophenyl)(3-((tert-butoxycarbonyl)oxy)-3-methylbutyl)(oxo)-l6-sulfaneylidene)carbamate 5 (300.00 mg, 0.68 mmol) and DIEA (440.06 mg, 3.40 mmol) in THF (5 mL) at 0° C. The resulting mixture was stirred at room temperature for 2 hours. Then the mixture was quenched with water (50 mL) and extracted with DCM (50 mL×3). The combined organic phases were washed with brine, dried over sodium sulfate, concentrated under vacuum. The residue was purified by flash column chromatography on silica gel (PE/EtOAc=1/1) to provide tert-butyl ((3-((tert-butoxycarbonyl)oxy)-3-methylbutyl)(3-(2-((6-fluoro-2-methylpyridin-3-yl)oxy)-4-methyl-5-(trifluoromethyl)nicotinamido)phenyl)(oxo)-l6-sulfaneylidene)carbamate 6 (400 mg, 74.41% yield) as a yellow oil. LCMS (ESI) calcd. for C₃₀H₃₅F₄N₄O₆S [M−Boc+H]⁺ m/z 655.22, found 655.30.

Step 2. 2-((6-fluoro-2-methylpyridin-3-yl)oxy)-N-(3-(3-hydroxy-3-methylbutylsulfonimidoyl)phenyl)-4-methyl-5-(trifluoromethyl)nicotinamide: A solution of tert-butyl ((3-((tert-butoxycarbonyl)oxy)-3-methylbutyl)(3-(2-((6-fluoro-2-methylpyridin-3-yl)oxy)-4-methyl-5-(trifluoromethyl)nicotinamido)phenyl)(oxo)-l6-sulfaneylidene)carbamate 6 (400 mg, 0.53 mmol) in DCM (6 mL) was added TFA (3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (30 mL×3). The combined organic phases were washed with brine, dried over sodium sulfate, and concentrated under vacuum. The residue was purified by prep-HPLC (Gemini 5 um C₁₈ column, 150*21.2 mm, eluting with 35% to 65% MeCN/H₂O containing 0.05% NH₃) to obtain to obtain 2-((6-fluoro-2-methylpyridin-3-yl)oxy)-N-(3-(3-hydroxy-3-methylbutylsulfonimidoyl)phenyl)-4-methyl-5-(trifluoromethyl)nicotinamide (120 mg, 39.30% yield) as a yellow solid. ¹H NMR (400 MHz, DMSO-d₆, ppm) δ 11.19 (s, 1H), 8.55 (s, 1H), 8.34 (s, 1H), 7.98-7.78 (m, 2H), 7.64 (dt, J=15.6, 7.7 Hz, 2H), 7.11 (dd, J=8.7, 3.3 Hz, 1H), 4.40 (s, 1H), 4.25 (s, 1H), 3.32 (s, 3H), 3.20-3.09 (m, 2H), 2.25 (s, 3H), 1.83-1.50 (m, 2H), 1.03 (s, 6H). LCMS (ESI) calcd. for C₂₅H₂₇F₄N₄O₄S [M+H]⁺ m/z 555.17, found 555.3.

### Example 43C

### (R)—N-(3-(N-(2-aminoethyl)-S-methylsulfonimidoyl)phenyl)-2-((6-fluoro-2-methylpyridin-3-yl)oxy)-4-methyl-5-(trifluoromethyl)nicotinamide

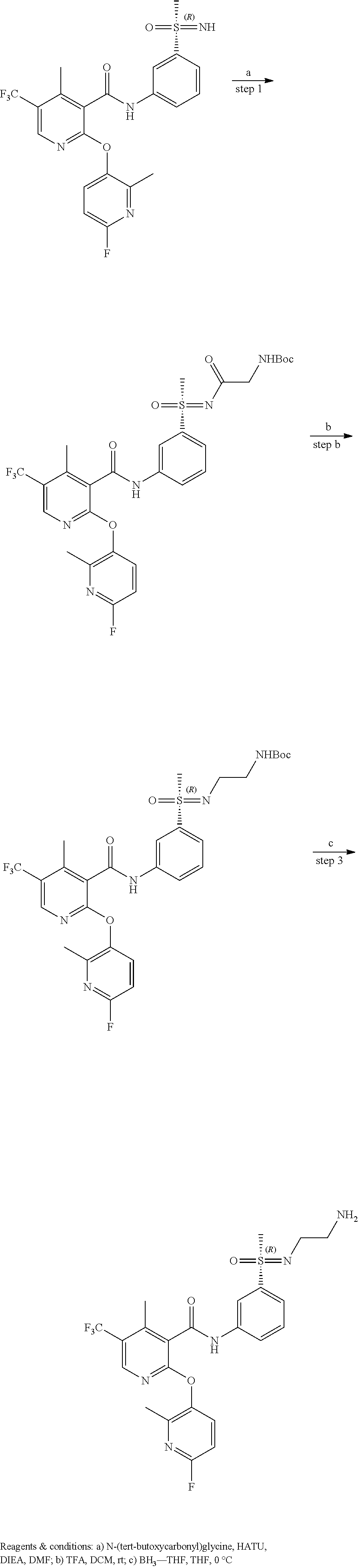

Step 1: tert-butyl (R)-(2-(((3-(2-((6-fluoro-2-methylpyridin-3-yl)oxy)-4-methyl-5-(trifluoromethyl)nicotinamido)phenyl)(methyl)(oxo)-λ⁶-sulfaneylidene)amino)-2-oxoethyl) carbamate: A mixture of (R)-2-((6-fluoro-2-methylpyridin-3-yl)oxy)-4-methyl-N-(3-(S-methylsulfonimidoyl)phenyl)-5-(trifluoromethyl)nicotinamide (100 mg, 0.21 mmol) and (tert-butoxycarbonyl)glycine (44 mg, 0.25 mmol) in DMF (5 mL) was added HATU (122 mg, 0.32 mmol) and DIEA (54 mg, 0.42 mmol). The mixture was stirred at room temperature for 4 hours. The reaction was monitored by LCMS. After the reaction was completed, the resulting solution was diluted with water (50 mL) and extracted with DCM (20 mL×3). The combined organic layers were washed with brine, dried over Na₂SO₄, concentrated under vacuum. The residue was purified by flash column chromatography on silica gel (PE/EtOAc=2/1 to 1/1) to give tert-butyl (R)-(2-(((3-(2-((6-fluoro-2-methylpyridin-3-yl)oxy)-4-methyl-5-(trifluoromethyl)nicotinamido) phenyl)(methyl)(oxo)-λ⁶-sulfaneylidene)amino)-2-oxoethyl)carbamate (100 mg, 74.4%) as a yellow solid. LCMS (ESI) calcd. for C₂₈H₃₀F₄N₅O₆S [M+H]⁺ m/z 640.19, found 640.05.

Step 2: (R)-2-((6-fluoro-2-methylpyridin-3-yl)oxy)-N-(3-(N-glycyl-S-methylsulfonimidoyl)phenyl)-4-methyl-5-(trifluoromethyl)nicotinamide. A solution of tert-butyl (R)-(2-(((3-(2-((6-fluoro-2-methylpyridin-3-yl)oxy)-4-methyl-5-(trifluoromethyl)nicotinamido) phenyl)(methyl)(oxo)-λ⁶-sulfaneylidene)amino)-2-oxoethyl)carbamate (100 mg, 0.15 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (20 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.05% NH₃) to give (R)-2-((6-fluoro-2-methylpyridin-3-yl)oxy)-N-(3-(N-glycyl-S-methylsulfonimidoyl)phenyl)-4-methyl-5-(trifluoromethyl)nicotinamide (36.6 mg, 45.2%) as a white solid. ¹H NMR (400 MHz, CD₃OD-d₄) δ 8.55 (t, J=1.9 Hz, 1H), 8.45 (s, 1H), 8.02-7.95 (m, 1H), 7.83 (d, J=8.7 Hz, 1H), 7.79-7.68 (m, 2H), 6.99 (dd, J=8.6, 3.2 Hz, 1H), 3.46 (d, J=9.1 Hz, 5H), 2.60 (s, 3H), 2.32 (s, 3H). LCMS (ESI) calcd. for C₂₃H₂₂F₄N₅O₄S [M+H]⁺ m/z 540.14, found 540.05.

Step 3: A solution of (R)-2-((6-fluoro-2-methylpyridin-3-yl)oxy)-N-(3-(N-glycyl-S-methylsulfonimidoyl)phenyl)-4-methyl-5-(trifluoromethyl)nicotinamide (250 mg, 0.46 mmol) in THF (15 mL) was added BH₃-THF (1.5 mL) at 0 CC. The reaction mixture was stirred at 0° C. for 1 hour. After the reaction was completed, the mixture was quenched with water and the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 40% to 95% MeCN/H₂O containing 0.05% NH₃·H₂O) to provide (R)—N-(3-(N-(2-aminoethyl)-S-methylsulfonimidoyl)phenyl)-2-((6-fluoro-2-methylpyridin-3-yl)oxy)-4-methyl-5-(trifluoromethyl)nicotinamide (24.5 mg, 9.4% yield) as a white solid. ¹H NMR (400 MHz, DMSO-d₆, ppm) δ 11.22 (s, 1H), 8.55 (s, 1H), 8.32 (d, J=9.7 Hz, 1H), 7.99-7.81 (m, 2H), 7.68-7.59 (m, 2H), 7.11 (d, J=8.5 Hz, 1H), 3.14 (d, J=1.4 Hz, 3H), 3.03-2.98 (m, 1H), 2.86-2.78 (m, 1H), 2.72-2.67 (m, 1H), 2.58 (t, J=6.3 Hz, 1H), 2.51 (s, 3H), 2.25 (s, 3H). LCMS (ESI) calcd. for C₂₃H₂₃F₄N503S [M+H]⁺ m/z 526.16, found 526.05.

### Example 44C

### (R)-5-((6-fluoro-2-methylpyridin-3-yl)oxy)-3-methyl-N-(3-(S-methylsulfonimidoyl)phenyl)-2-(trifluoromethyl)isonicotinamide

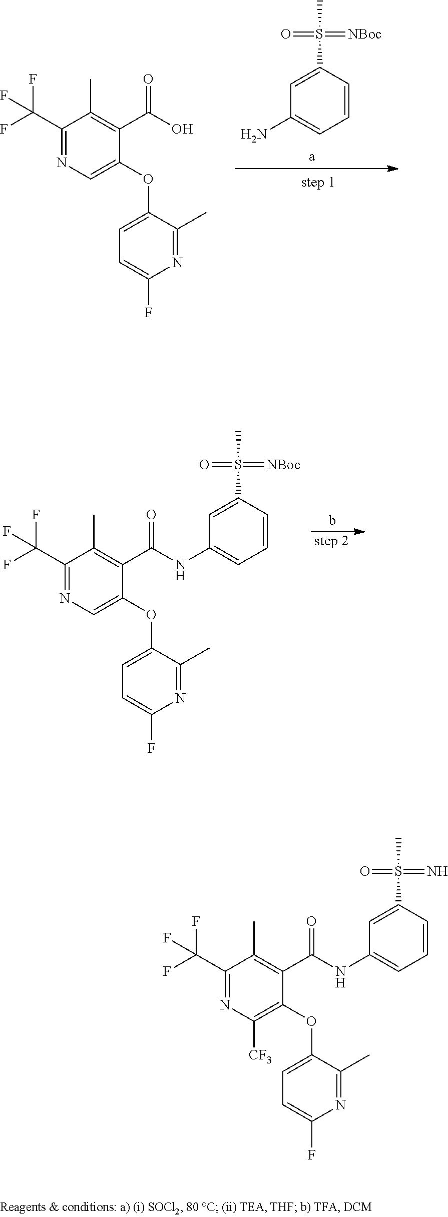

Step 1: A solution of 5-((6-fluoro-2-methylpyridin-3-yl)oxy)-3-methyl-2-(trifluoromethyl)isonicotinic acid (110 mg, 0.33 mmol) in SOCl₂ (2 mL) was heated at 80° C. for 0.5 h. The solution was concentrated under vacuum to provide the chloride intermediate. Then the chloride intermediate dissolved in THE (1 mL) was added to a solution of tert-butyl (R)-((3-aminophenyl)(methyl)(oxo)-l6-sulfaneylidene)carbamate (108 mg, 0.40 mmol) and TEA (101 mg, 0.99 mmol) in THE (1 mL) at 0° C. The resulting mixture was stirred at room temperature for 2 hours. Then the mixture was quenched with water (50 mL) and extracted with DCM (50 mL×3). The combined organic phases were washed with brine, dried over sodium sulfate, concentrated under vacuum. The residue was purified by flash column chromatography on silica gel (PE/EtOAc=1/1) to provide tert-butyl (R)-((3-(5-((6-fluoro-2-methylpyridin-3-yl)oxy)-3-methyl-2-(trifluoromethyl)isonicotinamido)phenyl)(methyl)(oxo)-l6-sulfaneylidene)carbamate (90 mg, 46.4% yield) as a yellow solid. LCMS (ESI) calcd. for C₂₆H₂₇F₄N₄O₅S [M+H]⁺ m/z 583.17, found 583.00.

Step 2: A solution of tert-butyl (R)-((3-(5-((6-fluoro-2-methylpyridin-3-yl)oxy)-3-methyl-2-(trifluoromethyl)isonicotinamido)phenyl)(methyl)(oxo)-l6-sulfaneylidene)carbamate (90 mg, 0.15 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.05% NH₃) to obtain (R)-5-((6-fluoro-2-methylpyridin-3-yl)oxy)-3-methyl-N-(3-(S-methylsulfonimidoyl)phenyl)-2-(trifluoromethyl)isonicotinamide (38.5 mg, 51.6% yield) as a white solid. ¹H NMR (400 MHz, DMSO-d₆, ppm) δ 11.21 (s, 1H), 8.34 (s, 1H), 8.18 (s, 1H), 7.89-7.67 (m, 3H), 7.61 (t, J=7.9 Hz, 1H), 7.11 (dd, J=8.6, 3.2 Hz, 1H), 4.25 (s, 1H), 3.07 (s, 3H), 2.48 (s, 3H), 2.32 (s, 3H). LCMS (ESI) calcd. for C₂₁H₁₉F₄N₄O₃S [M+H]⁺ m/z 483.11, found 482.95.

### Example 45C

### (R)-3-chloro-5-((6-fluoro-2-methylpyridin-3-yl)oxy)-N-(3-(S-methylsulfonimidoyl)phenyl)-2-(trifluoromethyl)isonicotinamide

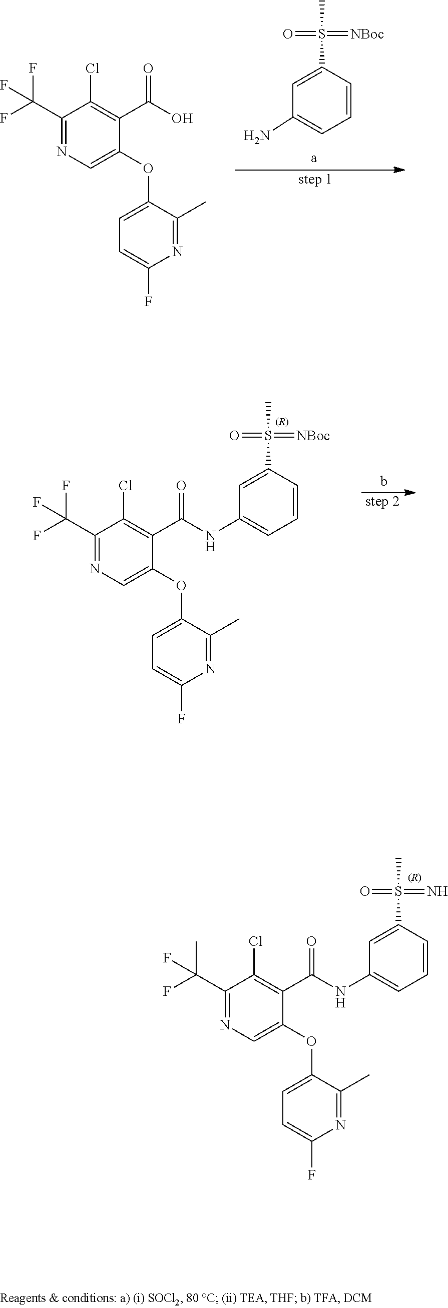

Step 1: A solution of 3-chloro-5-((6-fluoro-2-methylpyridin-3-yl)oxy)-2-(trifluoromethyl)isonicotinic acid (160 mg, 0.46 mmol) in SOCl₂ (2 mL) was heated to 80° C. for 0.5 h. The solution was concentrated under vacuum to provide the chloride intermediate. Then the chloride intermediate dissolved in THE (1 mL) was added to a solution of tert-butyl (R)-((3-aminophenyl)(methyl)(oxo)-l6-sulfaneylidene)carbamate (247 mg, 0.91 mmol) and TEA (231 mg, 2.28 mmol) in THE (1 mL) at 0° C. The resulting mixture was stirred at room temperature for 2 hours. Then the mixture was quenched with water (50 mL) and extracted with DCM (50 mL×3). The combined organic phases were washed with brine, dried over sodium sulfate, concentrated under vacuum. The residue was purified by flash column chromatography on silica gel (PE/EtOAc=1/1) to provide tert-butyl (R)-((3-(3-chloro-5-((6-fluoro-2-methylpyridin-3-yl)oxy)-2-(trifluoromethyl)isonicotinamido)phenyl)(methyl)(oxo)-l6-sulfaneylidene)carbamate (150 mg, 54.5% yield) as a yellow oil. LCMS (ESI) calcd. for C₂₅H₂₃ClF₄N₄O₅SNa [M+Na]⁺ m/z 625.09, found 625.00.

Step 2: A solution of tert-butyl (R)-((3-(3-chloro-5-((6-fluoro-2-methylpyridin-3-yl)oxy)-2-(trifluoromethyl)isonicotinamido)phenyl)(methyl)(oxo)-l6-sulfaneylidene)carbamate (150 mg, 0.25 mmol) in DCM (5 mL) was added TFA (0.5 mL) at room temperature. The reaction mixture was stirred at room temperature for 2 hours. After the reaction was completed, the solution was quenched with saturated aqueous NaHCO₃ (10 mL) and extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.05% NH₃) to obtain (R)-3-chloro-5-((6-fluoro-2-methylpyridin-3-yl)oxy)-N-(3-(S-methylsulfonimidoyl)phenyl)-2-(trifluoromethyl) isonicotinamide (100 mg, 80.0% yield) as a white solid. ¹H NMR (400 MHz, DMSO-d₆, ppm) δ 11.36 (s, 1H), 8.37 (s, 1H), 8.31 (t, J=1.8 Hz, 1H), 7.88 (dd, J=8.7, 6.7 Hz, 1H), 7.83 (d, J=8.1 Hz, 1H), 7.72 (d, J=8.2 Hz, 1H), 7.62 (t, J=7.9 Hz, 1H), 7.14 (dd, J=8.7, 3.3 Hz, 1H), 4.27 (s, 1H), 3.07 (s, 3H), 2.33 (s, 3H). LCMS (ESI) calcd. for C₂₀H₁₆ClF₄N₄O₃S [M+H]⁺ m/z 503.05, found 503.00.

### Example 46C and Example 47C

### (R)-2-((6-fluoro-2-methylpyridin-3-yl)oxy)-N,4-dimethyl-N-(3-(S-methylsulfonimidoyl)phenyl)-5-(trifluoromethyl)nicotinamide & (S)-2-((6-fluoro-2-methylpyridin-3-yl)oxy)-N,4-dimethyl-N-(3-(S-methylsulfonimidoyl)phenyl)-5-(trifluoromethyl)nicotinamide

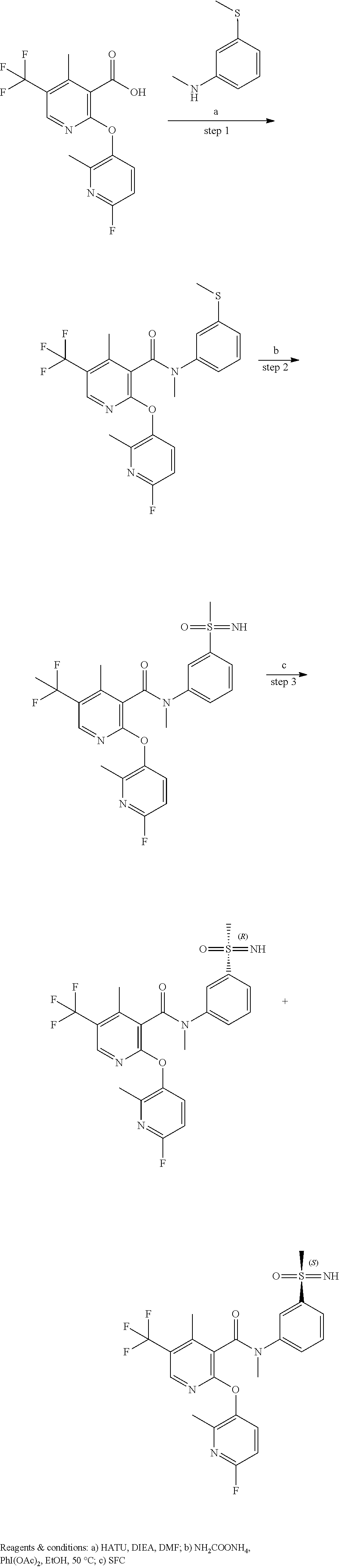

Step 1: A solution of N-methyl-3-(methylthio) aniline (204 mg, 1.33 mmol) in DMF (10 mL) was added 2-((6-fluoro-2-methylpyridin-3-yl)oxy)-4-methyl-5-(trifluoromethyl)nicotinic acid (400 mg, 1.21 mmol), HATU (599 mg, 1.58 mmol) and DIEA (501 mg, 3.88 mmol) at room temperature. The mixture was stirred at room temperature for 16 hours. After the reaction was completed, the mixture was cooled to room temperature and the mixture was diluted with water (20 mL) and extracted with DCM (20 mL×3). The combine organic phases were washed with brine, dried over sodium sulfate, concentrated under vacuum. The residue was purified by flash column chromatography on silica gel (PE/EtOAc=1/1) to give 2-((6-fluoro-2-methylpyridin-3-yl)oxy)-N,4-dimethyl-N-(3-(methylthio)phenyl)-5-(trifluoromethyl)nicotinamide (540 mg, 95.91% yield) as a white oil. LCMS (ESI) calcd. for C₂₂H₂₀F₄N₃O₂S [M+H]⁺ m/z 466.12, found 465.90.

Step 2: To a solution of 2-((6-fluoro-2-methylpyridin-3-yl)oxy)-N,4-dimethyl-N-(3-(methylthio)phenyl)-5-(trifluoromethyl)nicotinamide (540 mg, 1.16 mmol) in EtOH (10 mL) was added PhI(OAc)₂ (747 mg, 2.32 mmol) and ammonium carbamate (134 mg, 1.74 mmol) at room temperature. The reaction mixture was heated at 50° C. for 1 hour. After the reaction was completed, the mixture was cooled to room temperature, diluted with water (10 mL), and extracted with DCM (10 mL×3). The combined organic phases were washed with brine, dried over sodium sulfate, concentrated under vacuum. The residue was first purified by flash column chromatography on silica gel (PE/EtOAc=1/1), then further purified by prep-HPLC (Gemini 5 um C₁₈ column, 150*21.2 mm, eluting with 30% to 90% MeCN/H₂O containing 0.1% formic acid) to afford 2-((6-fluoro-2-methylpyridin-3-yl)oxy)-N,4-dimethyl-N-(3-(S-methylsulfonimidoyl) phenyl)-5-(trifluoromethyl)nicotinamide (120 mg, 41.67% yield, 97.2% purity) as a white solid. LCMS (ESI) calcd. for C₂₂H₂₁F₄N₄O₃S [M+H]⁺ m/z 497.13, found 497.10.

Step 3: 2-((6-fluoro-2-methylpyridin-3-yl)oxy)-N,4-dimethyl-N-(3-(S-methylsulfonimidoyl)phenyl)-5-(trifluoromethyl)nicotinamide was purified by Chiral-Prep-HPLC (DAICEL IH, 4.6*250 mm, eluting with 70/30 CO₂-MeOH containing 0.1% NH₃) to separate (R)- and (S)-2-((6-fluoro-2-methylpyridin-3-yl)oxy)-N,4-dimethyl-N-(3-(S-methylsulfonimidoyl) phenyl)-5-(trifluoromethyl)nicotinamide. First eluting product (Example 46C): (43.00 mg, 99.70% purity). Second eluting product (Example 47C): (45.00 mg, 98.0% purity), ¹H NMR (400 MHz, DMSO-d₆, ppm) δ 8.30 (s, 1H), 7.86-7.78 (m, 2H), 7.76-7.67 (m, 2H), 7.61 (t, J=7.8 Hz, 1H), 7.10 (dt, J=8.2, 3.9 Hz, 1H), 4.30 (s, 1H), 3.51 (s, 3H), 2.89 (s, 3H), 2.39 (s, 3H), 2.11 (s, 3H). LCMS (ESI) calcd. for C₂₂H₂₁F₄N₄O₃S [M+H]⁺ m/z 497.13, found 497.10.

### Example 48C

### 5-(difluoromethyl)-2-((6-fluoro-2-methylpyridin-3-yl)oxy)-4-methyl-N-(2-(S-methylsulfonimidoyl)pyridin-4-yl)nicotinamide

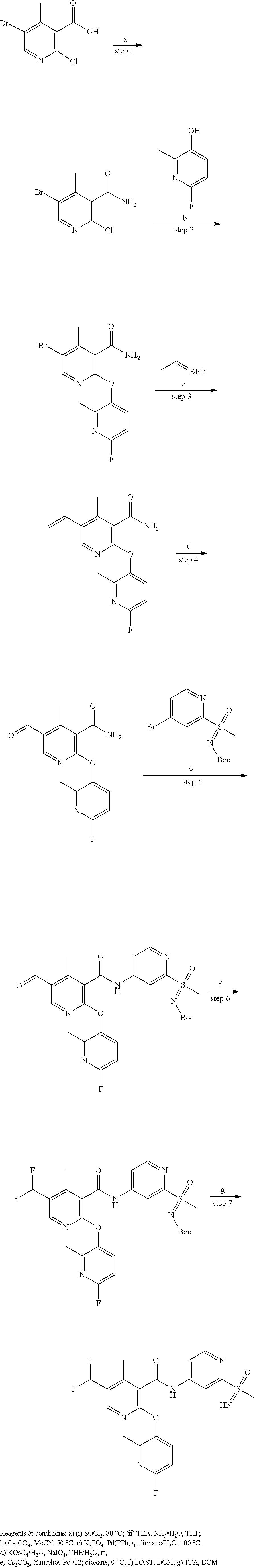

Step 1: A solution of 5-bromo-2-chloro-4-methylnicotinic acid (1.5 g, 6.05 mmol) in SOCl₂ (15 mL) was heated to 80° C. and stirred for 0.5 hour. The solution was concentrated under vacuum to provide the chloride intermediate. Then the chloride intermediate dissolved in THF (5 mL) was added to a solution of TEA (1.82 g, 18.02 mmol) in NH₃H₂O (30 mL) and at 0° C. The resulting mixture was stirred at 25° C. for 1 hour. Then the mixture was quenched with water (50 mL) and extracted with DCM (100 mL×3). The combined organic layers were washed with brine, dried over sodium sulfate, concentrated under vacuum. The residue was purified by flash column chromatography on silica gel (PE/EtOAc=1/1) to provide 5-bromo-2-chloro-4-methylnicotinamide (1.2 g, 81% yield) as white oil. LCMS (ESI) calcd. for C₇H₇BrClN₂O [M+H]⁺ m/z 248.95, found 250.90.

Step 2: A solution of 5-bromo-2-chloro-4-methylnicotinamide (1.2 g, 4.86 mmol), 6-fluoro-2-methylpyridin-3-ol (746 mg, 5.87 mmol) and Cs₂CO₃ (4.68 g, 14.27 mmol) in MeCN (20 mL) was heated to 80° C. and stirred for 2 hours. After the reaction was completed, the mixture was cooled to room temperature and filtered through celite. The filtrate was concentrated and residue was purified by flash column chromatography on silica gel (PE/EtOAc=10/1) to provide 5-bromo-2-((6-fluoro-2-methylpyridin-3-yl)oxy)-4-methylnicotinamide (1.1 g, 67.6% yield) as a white solid. LCMS (ESI) calcd. for C₁₃H₁₂BrFN₃O₂ [M+H]⁺ m/z 340.01, found 341.85.

Step 3: A mixture of 5-bromo-2-((6-fluoro-2-methylpyridin-3-yl)oxy)-4-methylnicotinamide (1.1 g, 3.24 mmol), 4,4,5,5-tetramethyl-2-vinyl-1,3,2-dioxaborolane (997 mg, 6.48 mmol), K₃PO₄ (1.37 g, 6.48 mmol) and Pd(PPh₃)₄ (187 mg, 0.16 mmol) in 1,4-dioxane/H₂O (10/3, 13 mL) was heated to 90° C. and stirred for 16 hours under nitrogen. After the reaction was completed, the mixture was cooled to room temperature and filtered through celite. The filtrate was concentrated and residue was purified by flash column chromatography on silica gel (PE/EtOAc=1/1) to provide 2-((6-fluoro-2-methylpyridin-3-yl)oxy)-4-methyl-5-vinylnicotinamide (800 mg, 86% yield) as a white solid. LCMS (ESI) calcd. for C₁₅H₁₅FN₃O₂ [M+H]⁺ m/z 288.12, found 288.00.

Step 4: A solution of 2-((6-fluoro-2-methylpyridin-3-yl)oxy)-4-methyl-5-vinylnicotinamide (750 mg, 2.61 mmol) and K₂OSO₄·2H₂O (190 mg, 0.5 mmol) in THF/H₂O (3/1, 12 mL) was added NaIO₄ (2.22 g, 10.33 mmol) at 0° C. The reaction mixture was stirred at 25° C. for 1 hour. After the reaction was completed, the resulting solution was diluted with EtOAc (30 mL) and washed with water (30 mL×3). The combined organic layers were washed with brine, dried over sodium sulfate, concentrated under vacuum. The residue was purified by flash column chromatography on silica gel (PE/EtOAc=1/1) to provide 2-((6-fluoro-2-methylpyridin-3-yl)oxy)-5-formyl-4-methylnicotinamide (600 mg, 79.9% yield) as a yellow solid. LCMS (ESI) calcd. for C₁₄H₁₃FN₃O₃ [M+H]⁺ m/z 290.10, found 289.95.

Step 5: A mixture of 2-((6-fluoro-2-methylpyridin-3-yl)oxy)-5-formyl-4-methylnicotinamide (400 mg, 1.38 mmol), tert-butyl ((4-bromopyridin-2-yl)(methyl)(oxo)-16-sulfaneylidene)carbamate (930 mg, 2.78 mmol), Cs₂CO₃ (1.35 g, 4.12 mmol) and Xantphos-Pd-G₂ (123 mg, 0.14 mmol) in 1,4-dioxane (10 mL) was stirred at 0° C. for 6 hours under nitrogen. After the reaction was completed, the mixture was filtered through celite. The filtrate was concentrated and residue was purified by flash column chromatography on silica gel (PE/EtOAc=1/2) to provide tert-butyl ((4-(2-((6-fluoro-2-methylpyridin-3-yl)oxy)-5-formyl-4-methylnicotinamido)pyridin-2-yl)(methyl)(oxo)-l6-sulfaneylidene)carbamate (300 mg, 39.5% yield) as a yellow solid. LCMS (ESI) calcd. for C₂₅H₂₇FN₅O₆S [M+H]⁺ m/z 544.17, found 544.00.

Step 6: A solution of tert-butyl ((4-(2-((6-fluoro-2-methylpyridin-3-yl)oxy)-5-formyl-4-methylnicotinamido)pyridin-2-yl)(methyl)(oxo)-l6-sulfaneylidene)carbamate (240 mg, 0.44 mmol) in DCM (8 mL) was added DAST (355 mg, 2.20 mmol) at 0° C. The reaction mixture was stirred at room temperature for 16 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by flash column chromatography on silica gel (PE/EtOAc=1/1) to provide tert-butyl ((4-(5-(difluoromethyl)-2-((6-fluoro-2-methylpyridin-3-yl)oxy)-4-methylnicotinamido)pyridin-2-yl)(methyl)(oxo)-l6-sulfaneylidene)carbamate (180 mg, 72% yield) as a white solid. LCMS (ESI) calcd. for C₂₅H₂₇F₃N₅O₅S [M+H]⁺ m/z 566.17, found 566.10.

Step 7: A solution of tert-butyl ((4-(5-(difluoromethyl)-2-((6-fluoro-2-methylpyridin-3-yl)oxy)-4-methylnicotinamido)pyridin-2-yl)(methyl)(oxo)-l6-sulfaneylidene)carbamate (180 mg, 0.32 mmol) in DCM (5 mL) was added TFA (1 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 40% to 90% MeCN/H₂O containing 0.05% NH₃) to provide 5-(difluoromethyl)-2-((6-fluoro-2-methylpyridin-3-yl)oxy)-4-methyl-N-(2-(S-methylsulfonimidoyl)pyridin-4-yl)nicotinamide (64.2 mg, 43.1% yield) as a white solid. ¹H NMR (400 MHz, DMSO-d₆, ppm) δ 11.59 (s, 1H), 8.64 (d, J=5.3 Hz, 1H), 8.47 (s, 1H), 8.35 (s, 1H), 7.86-7.77 (m, 2H), 7.41-7.14 (m, 1H), 7.10 (dd, J=8.7, 3.3 Hz, 1H), 4.40 (s, 1H), 3.15 (s, 3H), 2.46 (s, 3H), 2.23 (s, 3H). LCMS (ESI) calcd. for C₂₀H₁₉F₃N₅O₃S [M+H]⁺ m/z 466.12, found 465.95.

The following compounds are prepared using the techniques of the intermediates and examples described above.

| Ex. No. | Structure | MS (m/z) | NMR data |
|---|---|---|---|
| 49C | 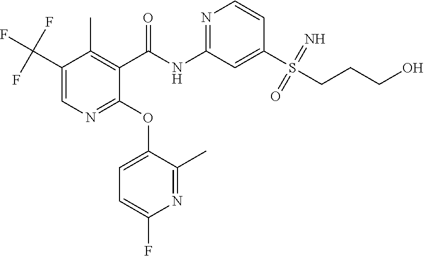 [SMILES: Cc1nc(F)ccc1Oc1ncc(C(F)(F)F)c(C)c1C(=O)Nc1cc(S(=N)(=O)CCCO)ccn1] | 528.2 | ¹H NMR (400 MHz, DMSO-d₆, ppm) δ 11.74 (s, 1 H), 8.76-8.49 (m, 3 H), 7.89-7.78 (m, 1 H), 7.62 (d, J = 4.3 Hz, 1 H), 7.11 (dd, J = 8.5, 3.1 Hz, 1 H), 4.67-4.55 (m, 2 H), 3.46-3.40 (m, 2 H), 3.27-3.22 (m, 2 H), 2.49-2.46 (m, 3 H), 2.24 (s, 3 H), 1.80-1.66 (m, 2 H). |
| 50C | 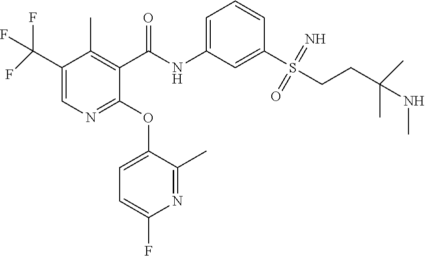 [SMILES: CNC(C)(C)CCS(=N)(=O)c1cccc(NC(=O)c2c(Oc3ccc(F)nc3C)ncc(C(F)(F)F)c2C)c1] | 568.2 | ¹H NMR (400 MHz, DMSO-d₆, ppm) δ 8.63 (t, J = 1.8 Hz, 1 H), 8.43 (s, 1 H), 7.88-7.81 (m, 2 H), 7.76-7.67 (m, 2 H), 6.97 (dd, J = 8.7, 3.0 Hz, 1 H), 3.48-3.37 (m, 2 H), 2.62 (s, 3 H), 2.58 (s, 3 H), 2.29 (s, 3 H), 2.24-2.16 (m, 1 H), 2.09-2.02 (m, 1 H), 1.32 (d, J = 4.9 Hz, 6 H). |
| 51C | 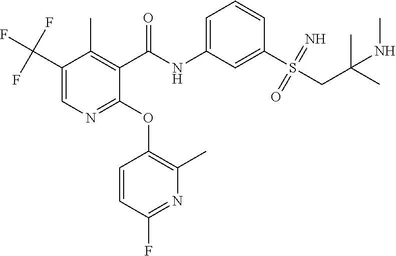 [SMILES: CNC(C)(C)CS(=N)(=O)c1cccc(NC(=O)c2c(Oc3ccc(F)nc3C)ncc(C(F)(F)F)c2C)c1] | 554.1 | ¹H NMR (400 MHz, DMSO-d₆, ppm) δ 11.32 (s, 1 H), 8.90 (s, 2 H), 8.54 (d, J = 9.6 Hz, 2 H), 7.92-7.80 (m, 2 H), 7.76-7.63 (m, 2 H), 7.12 (d, J = 6.2 Hz, 1 H), 3.77 (s, 3 H), 3.59-3.54 (m, 2 H), 2.61 (s, 3 H), 2.25 (s, 3 H), 1.59 (s, 3 H), 1.29 (s, 3 H). |
| 52C | 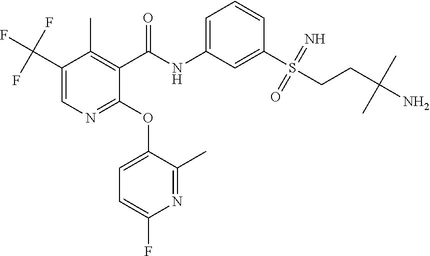 [SMILES: Cc1nc(F)ccc1Oc1ncc(C(F)(F)F)c(C)c1C(=O)Nc1cccc(S(=N)(=O)CCC(C)(C)N)c1] | 554.2 | ¹H NMR (400 MHz, DMSO-d₆, ppm) δ 11.20 (s, 1 H), 8.55 (s, 1 H), 8.33 (d, J = 1.7 Hz, 1 H), 7.92 (d, J = 7.5 Hz, 1 H), 7.87-7.80 (m, 1 H), 7.68- 7.58 (m, 2 H), 7.11 (dd, J = 8.5, 3.3 Hz, 1 H), 4.22 (s, 1 H), 3.19-3.11 (m, 2 H), 2.50 (s, 3 H), 2.24 (s, 3 H), 1.63-1.49 (m, 2 H), 0.94 (d, J = 3.3 Hz, 6 H). |
| 53C | 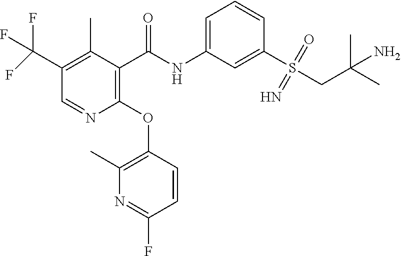 [SMILES: Cc1nc(F)ccc1Oc1ncc(C(F)(F)F)c(C)c1C(=O)Nc1cccc(S(=N)(=O)CC(C)(C)N)c1] | 540.2 | ¹H NMR (400 MHz, DMSO-d₆, ppm) δ 11.19 (s, 1 H), 8.55 (s, 1 H), 8.41 (s, 1 H), 7.84 (t, J = 7.8 Hz, 2 H), 7.70-7.59 (m, 2 H), 7.12 (dd, J = 8.7, 3.1 Hz, 1 H), 3.30 (s, 3 H), 3.25 (d, J = 5.5 Hz, 2 H), 2.24 (s, 3 H), 1.24 (s, 3 H), 1.12 (s, 3 H). |
| 54C | 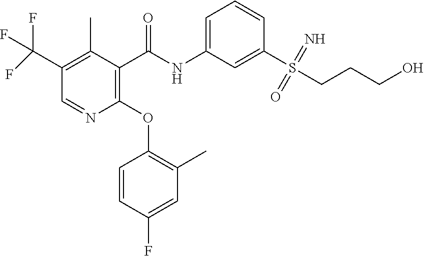 [SMILES: Cc1cc(F)ccc1Oc1ncc(C(F)(F)F)c(C)c1C(=O)Nc1cccc(S(=N)(=O)CCCO)c1] | 526.1 | ¹H NMR (400 MHz, DMSO-d₆, ppm) δ 11.16 (s, 1 H), 8.52 (s, 1 H), 8.36 (s, 1 H), 7.91 (d, J = 7.5 Hz, 1 H), 7.66-7.58 (m, 2 H), 7.24-7.15 (m, 2 H), 7.12-7.05 (m, 1 H), 4.57 (s, 1 H), 4.25 (s, 1 H), 3.40 (t, J = 6.2 Hz, 2 H), 3.18-3.11 (m, 2 H), 2.49-2.48 (m, 3 H), 2.08 (s, 3 H), 1.74-1.62 (m, 2 H). |
| 55C | 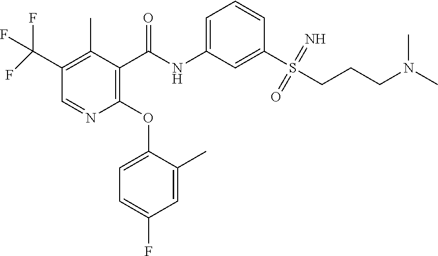 [SMILES: Cc1cc(F)ccc1Oc1ncc(C(F)(F)F)c(C)c1C(=O)Nc1cccc(S(=N)(=O)CCCN(C)C)c1] | 553.1 | ¹H NMR (400 MHz, DMSO-d₆, ppm, formate salt) δ 11.16 (s, 1 H), 8.52 (s, 1 H), 8.37 (s, 1 H), 8.27-8.11 (m, 1 H), 7.90 (d, J = 7.6 Hz, 1 H), 7.65- 7.60 (m, 2 H), 7.23-7.16 (m, 2 H), 7.09 (td, J = 8.5, 3.0 Hz, 1 H), 3.13 (t, J = 7.7 Hz, 2 H), 2.48 (s, 3 H), 2.25 (t, J = 6.7 Hz, 2 H), 2.08 (s, 3 H), 2.06 (s, 6 H), 1.79-1.57 (m, 2 H). |
| 56C | 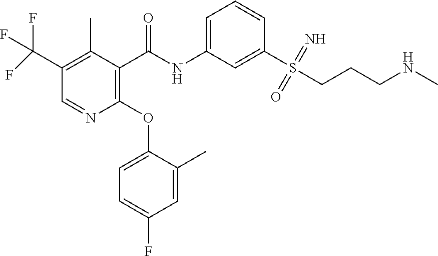 [SMILES: CNCCCS(=N)(=O)c1cccc(NC(=O)c2c(Oc3ccc(F)cc3C)ncc(C(F)(F)F)c2C)c1] | 539.1 | ¹H NMR (400 MHz, DMSO-d₆, ppm, formate salt) δ 11.28 (d, J = 6.2 Hz, 1 H), 8.51 (s, 1 H), 8.41 (s, 1 H), 8.30 (s, 1 H), 7.90 (d, J = 7.2 Hz, 1 H), 7.71-7.56 (m, 2 H), 7.19 (dt, J = 9.1, 4.5 Hz, 2 H), 7.19-6.98 (m, 1 H), 3.24 (d, J = 7.4 Hz, 2 H), 2.80 (d, J = 6.1 Hz, 2 H), 2.48 (s, 3 H), 2.38 (t, J = 5.2 Hz, 3 H), 2.08 (s, 3 H), 1.87- 1.78 (m, 2 H). |
| 57C | 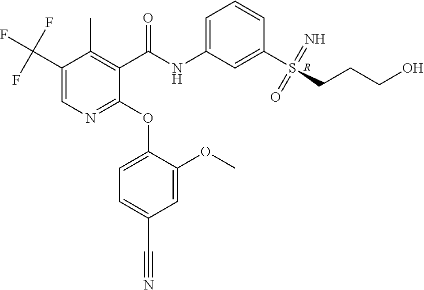 | 549.2 | ¹H NMR (400 MHz, DMSO-d₆, ppm) δ 11.13 (s, 1 H), 8.51 (s, 1 H), 8.36 (s, 1 H), 7.91 (d, J = 7.5 Hz, 1 H), 7.69-7.60 (m, 3 H), 7.51 (dd, J = 8.2, 1.7 Hz, 1 H), 7.41 (d, J = 8.2 Hz, 1 H), 4.58 (t, J = 5.3 Hz, 1 H), 4.25 (s, 1 H), 3.77 (s, 3 H), 3.40 (q, J = 6.0 Hz, 2 H), 3.17-3.12 (m, 2 H), 2.49- 2.47 (m, 3 H), 1.75-1.63 (m, 2 H). |
| 58C | 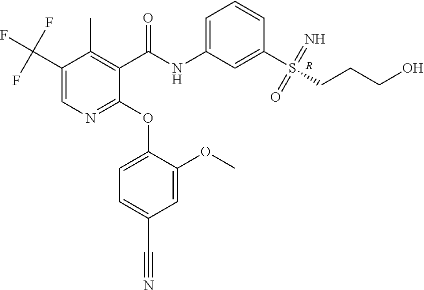 | 549.2 | ¹H NMR (400 MHz, DMSO-d₆, ppm) δ 11.13 (s, 1 H), 8.51 (s, 1 H), 8.36 (s, 1 H), 7.91 (d, J = 7.6 Hz, 1 H), 7.69-7.60 (m, 3 H), 7.51 (dd, J = 8.2, 1.8 Hz, 1 H), 7.41 (d, J = 8.2 Hz, 1 H), 4.58 (t, J = 5.2 Hz, 1 H), 4.25 (s, 1 H), 3.77 (s, 3 H), 3.42-3.37 (m, 2 H), 3.17-3.11 (m, 2 H), 2.49 (s, 3 H), 1.76-1.61 (m, 2 H). |
| 59C | 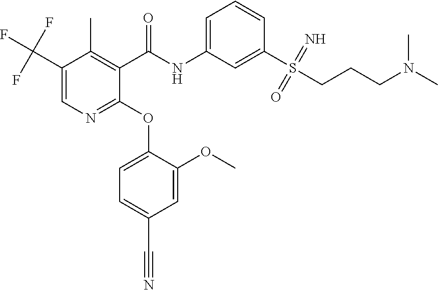 [SMILES: COc1cc(C#N)ccc1Oc1ncc(C(F)(F)F)c(C)c1C(=O)Nc1cccc(S(=N)(=O)CCCN(C)C)c1] | 576.2 | ¹H NMR (400 MHz, DMSO-d₆, ppm) δ 11.19 (s, 1 H), 8.51 (d, J = 7.6 Hz, 2 H), 7.84 (d, J = 7.7 Hz, 1 H), 7.70- 7.62 (m, 3 H), 7.52 (dd, J = 8.2, 1.6 Hz, 1 H), 7.41 (d, J = 8.2 Hz, 1 H), 3.77 (s, 3 H), 3.27 (t, J = 7.4 Hz, 2 H), 3.15 (t, J = 7.8 Hz, 2 H), 2.75 (s, 6 H), 2.48 (s, 3 H), 2.04-1.94 (m, 2 H). |
| 60C | 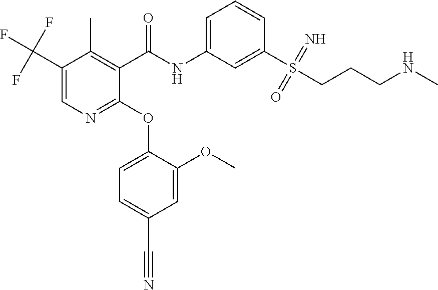 [SMILES: CNCCCS(=N)(=O)c1cccc(NC(=O)c2c(Oc3ccc(C#N)cc3OC)ncc(C(F)(F)F)c2C)c1] | 562.2 | ¹H NMR (400 MHz, DMSO-d₆, ppm) δ 11.13 (s, 1 H), 8.51 (s, 1 H), 8.36 (s, 1 H), 7.90 (d, J = 7.6 Hz, 1 H), 7.68-7.58 (m, 3 H), 7.51 (dd, J = 8.2, 1.7 Hz, 1 H), 7.41 (d, J = 8.2 Hz, 1 H), 3.77 (s, 3 H), 3.15 (t, J = 7.9 Hz, 2 H), 2.49-2.43 (m, 6 H), 2.16 (s, 3 H), 1.71-1.61 (m, 2 H). |
| 61C | 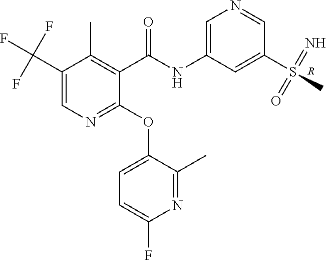 | 484.2 | ¹H NMR (400 MHz, DMSO-d₆, ppm) δ 11.47 (s, 1 H), 8.97 (d, J = 2.3 Hz, 1 H), 8.84 (d, J = 1.9 Hz, 1 H), 8.79 (t, J = 2.2 Hz, 1 H), 8.57 (s, 1 H), 7.85 (dd, J = 8.5, 7.0 Hz, 1 H), 7.12 (dd, J = 8.7, 3.4 Hz, 1 H), 4.56 (s, 1 H), 3.18 (s, 3 H), 2.50 (s, 3 H), 2.25 (s, 3 H). |
| 62C | 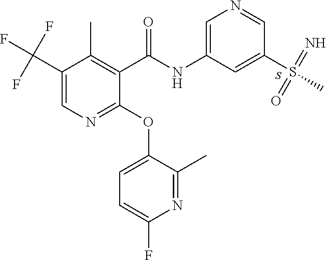 | 484.2 | ¹H NMR (400 MHz, DMSO-d₆, ppm) δ 11.46 (s, 1 H), 8.97 (d, J = 2.3 Hz, 1 H), 8.84 (d, J = 1.9 Hz, 1 H), 8.78 (t, J = 2.1 Hz, 1 H), 8.57 (s, 1 H), 7.85 (dd, J = 8.5, 7.0 Hz, 1 H), 7.12 (dd, J = 8.7, 3.4 Hz, 1 H), 4.57 (s, 1 H), 3.18 (s, 3 H), 2.50 (s, 3 H), 2.25 (s, 3 H). |
| 63C | 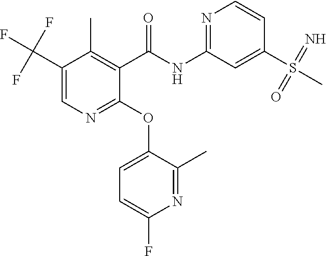 [SMILES: Cc1nc(F)ccc1Oc1ncc(C(F)(F)F)c(C)c1C(=O)Nc1cc(S(C)(=N)=O)ccn1] | 484.1 | ¹H NMR (400 MHz, DMSO-d₆, ppm) δ 11.74 (s, 1 H), 8.77 (s, 1 H), 8.63 (d, J = 5.0 Hz, 1 H), 8.53 (s, 1 H), 7.84 (t, J = 7.6 Hz, 1 H), 7.68 (d, J = 5.0 Hz, 1 H), 7.11 (dd, J = 8.6, 3.0 Hz, 1 H), 4.60 (s, 1 H), 3.16 (s, 3 H), 2.50-2.48 (m, 3 H), 2.24 (s, 3 H). |
| 64C | 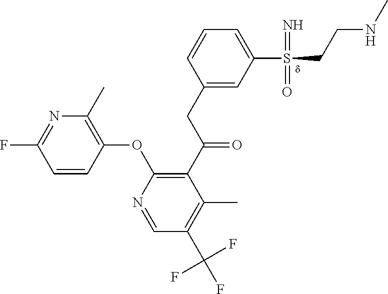 [SMILES: CNCC[S@@](=N)(=O)c1cccc(CC(=O)c2c(Oc3ccc(F)nc3C)ncc(C(F)(F)F)c2C)c1] | 526.4 | ¹H NMR (400 MHz, DMSO-d₆, ppm) δ 11.20 (s, 1 H), 8.55 (s, 1 H), 8.37 (s, 1 H), 7.95-7.81 (m, 2 H), 7.70- 7.58 (m, 2 H), 7.11 (dd, J = 8.7, 3.4 Hz, 1 H), 4.35 (s, 1 H), 3.32 (s, 3 H), 3.28-3.21 (m, 2 H), 2.78-2.66 (m, 2 H), 2.24 (s, 3 H), 2.19 (s, 3 H). |
| 65C | 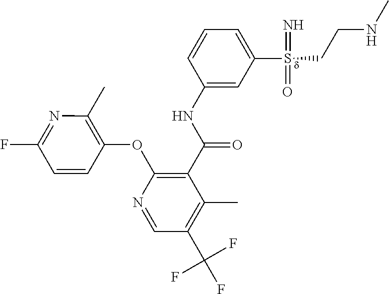 [SMILES: CNCC[S@](=N)(=O)c1cccc(NC(=O)c2c(Oc3ccc(F)nc3C)ncc(C(F)(F)F)c2C)c1] | 526.4 | ¹H NMR (400 MHz, DMSO-d₆, ppm) δ 11.20 (s, 1 H), 8.55 (s, 1 H), 8.37 (s, 1 H), 7.94-7.82 (m, 2 H), 7.70- 7.59 (m, 2 H), 7.11 (dd, J = 8.7, 3.4 Hz, 1 H), 4.34 (s, 1 H), 3.32 (s, 3 H), 3.27-3.21 (m, 2 H), 2.77-2.67 (m, 2 H), 2.24 (s, 3 H), 2.19 (s, 3 H). |
| 66C | 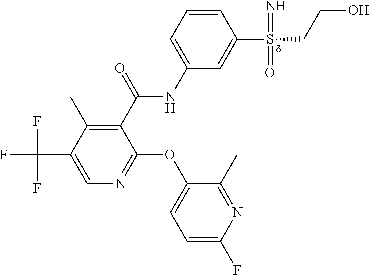 [SMILES: Cc1nc(F)ccc1Oc1ncc(C(F)(F)F)c(C)c1C(=O)Nc1cccc([S@@](=N)(=O)CCO)c1] | 513.1 | ¹H NMR (400 MHz, DMSO-d₆, ppm) δ 11.19 (s, 1 H), 8.55 (s, 1 H), 8.36 (s, 1 H), 7.93-7.82 (m, 2 H), 7.69- 7.59 (m, 2 H), 7.11 (dd, J = 8.6, 3.3 Hz, 1 H), 4.84 (s, 1 H), 4.37 (s, 1 H), 3.67 (s, 2 H), 3.29 (d, J = 6.7 Hz, 2 H), 2.51 (s, 3 H), 2.25 (s, 3 H). |
| 67C | 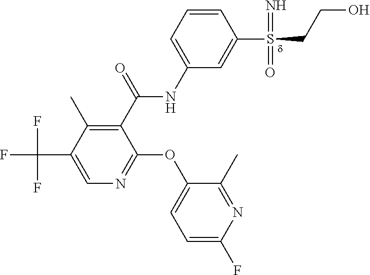 | 513.1 | ¹H NMR (400 MHz, DMSO-d₆, ppm) δ 11.20 (s, 1 H), 8.55 (s, 1 H), 8.36 (s, 1 H), 7.93-7.82 (m, 2 H), 7.69- 7.59 (m, 2 H), 7.11 (dd, J = 8.6, 3.3 Hz, 1 H), 4.84 (s, 1 H), 4.37 (s, 1 H), 3.67 (s, 2 H), 3.29 (d, J = 6.4 Hz, 2 H), 2.50 (s, 3 H), 2.25 (s, 3 H). |
| 68C | 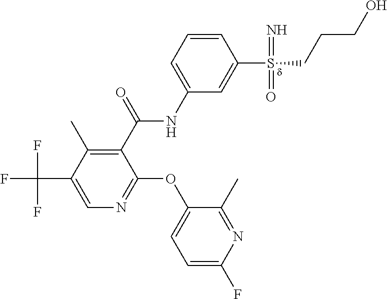 | 527.0 | ¹H NMR (400 MHz, DMSO-d₆, ppm) δ 11.20 (s, 1 H), 8.55 (s, 1 H), 8.35 (s, 1 H), 7.96-7.81 (m, 2 H), 7.67- 7.60 (m, 2 H), 7.11 (dd, J = 8.7, 3.3 Hz, 1 H), 4.59 (t, J = 5.3 Hz, 1 H), 4.26 (s, 1 H), 3.43-3.39 (m, 2 H), 3.17-3.12 (m, 2 H), 2.51 (s, 3 H), 2.24 (s, 3 H), 1.69 (m, 2 H). |
| 69C | 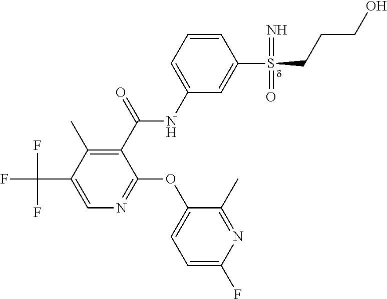 [SMILES: Cc1nc(F)ccc1Oc1ncc(C(F)(F)F)c(C)c1C(=O)Nc1cccc([S@](=N)(=O)CCCO)c1] | 527.0 | ¹H NMR (400 MHz, DMSO-d₆, ppm) 11.19 (s, 1 H), 8.55 (s, 1 H), 8.35 (s, 1 H), 7.94-7.81 (m, 2 H), 7.69-7.58 (m, 2 H), 7.11 (dd, J = 8.7, 3.4 Hz, 1 H), 4.58 (t, J = 5.1 Hz, 1 H), 4.26 (s, 1 H), 3.41-3.38 (m, 2 H), 3.18-3.11 (m, 2 H), 2.51 (s, 3 H), 2.24 (s, 3 H), 1.74-1.62 (m, 2 H). |
| 70C | 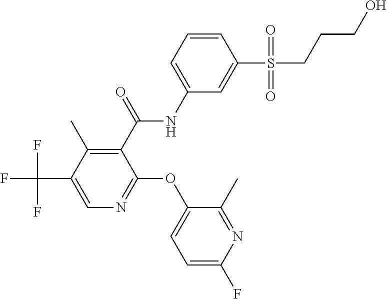 [SMILES: Cc1nc(F)ccc1Oc1ncc(C(F)(F)F)c(C)c1C(=O)Nc1cccc(S(=O)(=O)CCCO)c1] | 528.0 | ¹H NMR (400 MHz, DMSO-d₆, ppm) δ 11.28 (s, 1 H), 8.55-8.37 (m, 2 H), 8.06-7.80 (m, 2 H), 7.67 (s, 2 H), 7.11 (dd, J = 8.7, 3.3 Hz, 1 H), 4.64 (s, 1 H), 3.43 (d, J = 4.0 Hz, 2 H), 3.29 (s, 2 H), 2.49 (s, 3 H), 2.24 (s, 3 H), 1.79-1.63 (m, 2 H). |
| 71C | 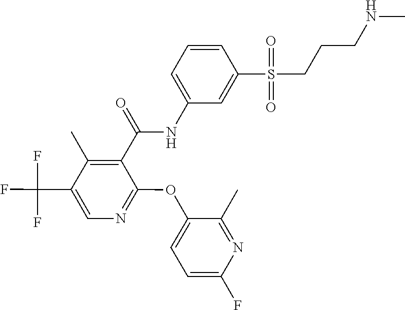 [SMILES: CNCCCS(=O)(=O)c1cccc(NC(=O)c2c(Oc3ccc(F)nc3C)ncc(C(F)(F)F)c2C)c1] | 541.4 | ¹H NMR (400 MHz, CDCl₃, ppm) δ 8.62 (s, 1 H), 8.40-8.30 (m, 2 H), 8.05 (s, 1 H), 7.70-7.58 (m, 3 H), 6.81 (dd, J = 8.6, 3.6 Hz, 1 H), 3.28- 3.17 (m, 2 H), 2.76 (t, J = 6.6 Hz, 2 H), 2.59 (s, 3 H), 2.43 (s, 3 H), 2.32 (s, 3 H), 2.03-1.92 (m, 2 H). |
| 72C | 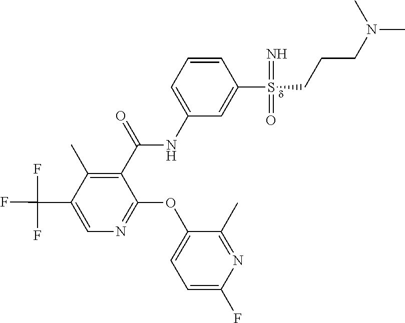 [SMILES: Cc1nc(F)ccc1Oc1ncc(C(F)(F)F)c(C)c1C(=O)Nc1cccc([S@@](=N)(=O)CCCN(C)C)c1] | 554.2 | ¹H NMR (400 MHz, DMSO-d₆, ppm) δ 11.19 (s, 1 H), 8.55 (s, 1 H), 8.36 (s, 1 H), 7.93-7.79 (m, 2 H), 7.69- 7.59 (m, 2 H), 7.11 (dd, J = 8.5, 3.2 Hz, 1 H), 4.28 (s, 1 H), 3.13 (t, J = 7.7 Hz, 2 H), 2.49-2.45 (m, 3 H), 2.25-2.16 (m, 5 H), 2.01 (s, 6 H), 1.72-1.60 (m, 2 H). |
| 73C | 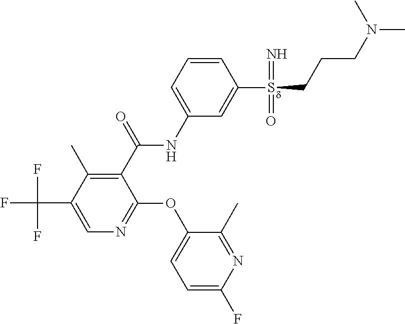 [SMILES: Cc1nc(F)ccc1Oc1ncc(C(F)(F)F)c(C)c1C(=O)Nc1cccc([S@](=N)(=O)CCCN(C)C)c1] | 554.2 | ¹H NMR (400 MHz, DMSO-d₆, ppm) δ 11.19 (s, 1 H), 8.55 (s, 1 H), 8.36 (s, 1 H), 7.95-7.79 (m, 2 H), 7.69- 7.58 (m, 2 H), 7.11 (dd, J = 8.6, 3.4 Hz, 1 H), 4.30 (s, 1 H), 3.13 (t, J = 7.8 Hz, 2 H), 2.49-2.44 (m, 3 H), 2.26-2.15 (m, 5 H), 2.03 (s, 6 H), 1.74-1.60 (m, 2 H). |
| 74C | 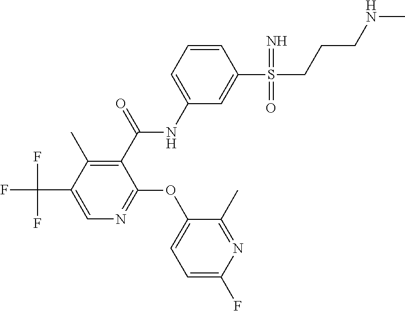 [SMILES: CNCCCS(=N)(=O)c1cccc(NC(=O)c2c(Oc3ccc(F)nc3C)ncc(C(F)(F)F)c2C)c1] | 540.1 | ¹H NMR (400 MHz, DMSO-d₆, ppm) δ 11.20 (s, 1 H), 8.45 (d, J = 77.3 Hz, 2H), 7.94-7.81 (m, 2 H), 7.68-7.59 (m, 2 H), 7.14-7.08 (m, 1 H), 4.24 (s, 1 H), 3.15 (t, J = 7.8 Hz, 2 H), 2.50- 2.47 (m, 3 H), 2.43 (t, J = 6.5 Hz, 2 H), 2.24 (s, 3 H), 2.15 (s, 3 H), 1.66 (dd, J = 14.5, 7.0 Hz, 2 H). |
| 75C | 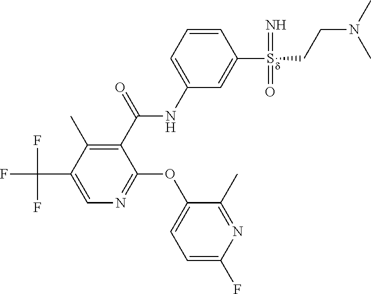 [SMILES: Cc1nc(F)ccc1Oc1ncc(C(F)(F)F)c(C)c1C(=O)Nc1cccc([S@@](=N)(=O)CCN(C)C)c1] | 540.1 | ¹H NMR (400 MHz, DMSO-d₆, ppm) δ 11.19 (s, 1 H), 8.55 (s, 1 H), 8.35 (s, 1 H), 7.95-7.80 (m, 2 H), 7.70- 7.59 (m, 2 H), 7.11 (dd, J = 8.7, 3.4 Hz, 1 H), 4.33 (s, 1 H), 3.30-3.23 (m, 2 H), 2.57-2.52 (m, 2 H), 2.51 (s, 3 H), 2.24 (s, 3 H), 2.05 (s, 6 H). |
| 76C | 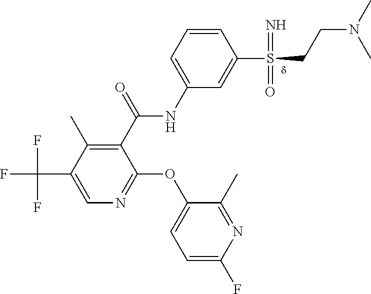 [SMILES: Cc1nc(F)ccc1Oc1ncc(C(F)(F)F)c(C)c1C(=O)Nc1cccc([S@](=N)(=O)CCN(C)C)c1] | 540.1 | ¹H NMR (400 MHz, DMSO-d₆, ppm) δ 11.19 (s, 1 H), 8.55 (s, 1 H), 8.36 (s, 1 H), 7.99-7.77 (m, 2 H), 7.72- 7.61 (m, 2 H), 7.11 (dd, J = 8.6, 3.2 Hz, 1 H), 4.33 (s, 1 H), 3.31-3.23 (m, 2 H), 2.62-2.51 (m, 2 H), 2.50 (s, 3 H), 2.24 (s, 3 H), 2.05 (d, J = 2.0 Hz, 6 H). |
| 77C | 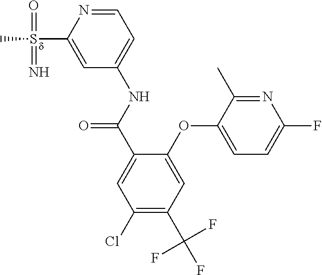 | 503.7 | ¹H NMR (400 MHz, DMSO-d₆, ppm) δ 11.44 (s, 1 H), 8.78 (s, 1 H), 8.65 (d, J = 5.4 Hz, 1 H), 8.37 (d, J = 1.7 Hz, 1 H), 7.97 (dd, J = 8.5, 7.0 Hz, 1 H), 7.79 (dd, J = 5.4, 1.8 Hz, 1 H), 7.19 (dd, J = 8.7, 3.4 Hz, 1 H), 4.41 (s, 1 H), 3.16 (s, 3 H), 2.28 (s, 3 H). |
| 78C | 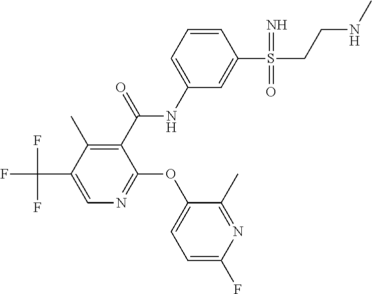 [SMILES: CNCCS(=N)(=O)c1cccc(NC(=O)c2c(Oc3ccc(F)nc3C)ncc(C(F)(F)F)c2C)c1] | 526.1 | ¹H NMR (400 MHz, DMSO-d₆, ppm) δ 11.22 (s, 1 H), 8.55 (s, 1 H), 8.37 (s, 1 H), 7.96-7.80 (m, 2 H), 7.71- 7.57 (m, 2 H), 7.12 (dd, J = 8.6, 3.3 Hz, 1 H), 4.35 (s, 1 H), 3.27-3.20 (m, 2 H), 2.76-2.67 (m, 2 H), 2.51 (s, 3 H), 2.29-2.16 (m, 6 H). |
| 79C | 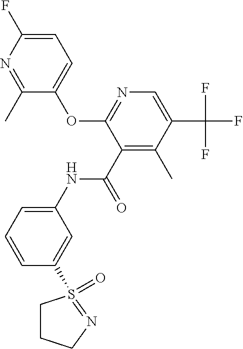 [SMILES: Cc1nc(F)ccc1Oc1ncc(C(F)(F)F)c(C)c1C(=O)Nc1cccc([S@]2(=O)=NCCC2)c1] | 509.1 | ¹H NMR (400 MHz, DMSO-d₆, ppm) δ 11.29 (s, 1 H), 8.56 (s, 1 H), 8.41 (s, 1 H), 7.97 (d, J = 3.4 Hz, 1 H), 7.90-7.80 (m, 1 H), 7.68 (d, J = 5.8 Hz, 2 H), 7.11 (dd, J = 8.7, 3.3 Hz, 1 H), 3.86 (dd, J = 10.6, 5.6 Hz, 1 H), 3.69 (dt, J = 10.0, 6.6 Hz, 1 H), 3.57 (dd, J = 14.7, 7.4 Hz, 2 H), 2.51 (s, 3 H), 2.33 (dd, J = 13.8, 6.7 Hz, 2 H), 2.24 (s, 3 H). |
| 80C | 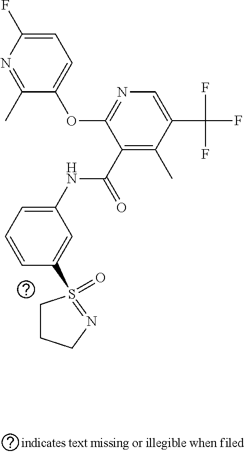 [SMILES: Cc1nc(F)ccc1Oc1ncc(C(F)(F)F)c(C)c1C(=O)Nc1cccc([S@@]2(=O)=NCCC2)c1] | 509.0 | ¹H NMR (400 MHz, DMSO-d₆, ppm) δ 11.26 (s, 1 H), 8.55 (s, 1 H), 8.38 (s, 1 H), 8.00-7.92 (m, 1 H), 7.84 (dd, J = 8.5, 7.0 Hz, 1 H), 7.73-7.58 (m, 2 H), 7.11 (dd, J = 8.7, 3.4 Hz, 1 H), 3.83 (dd, J = 10.5, 5.2 Hz, 1 H), 3.67 (dt, J = 10.2, 6.5 Hz, 1 H), 3.47 (dd, J = 15.4, 7.5 Hz, 2 H), 2.51 (s, 3 H), 2.35-2.17 (m, 5 H). |
| 81C | 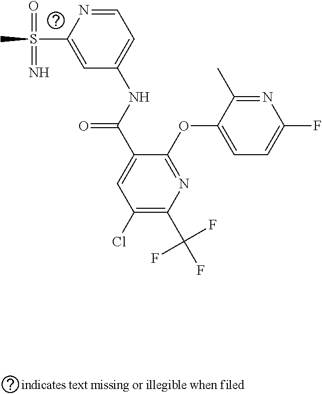 [SMILES: Cc1nc(F)ccc1Oc1nc(C(F)(F)F)c(Cl)cc1C(=O)Nc1ccnc([S@](C)(=N)=O)c1] | 504.0 | ¹H NMR (400 MHz, DMSO-d₆, ppm) δ 11.46 (s, 1 H), 8.78 (s, 1 H), 8.66 (d, J = 5.4 Hz, 1 H), 8.38 (d, J = 1.8 Hz, 1 H), 7.97 (dd, J = 8.6, 6.9 Hz, 1 H), 7.79 (dd, J-5.4, 1.9 Hz, 1 H), 7.19 (dd, J-8.7, 3.4 Hz, 1 H), 4.45 (s, 1 H), 3.17 (s, 3 H), 2.29 (s, 3 H). |
| 82C | 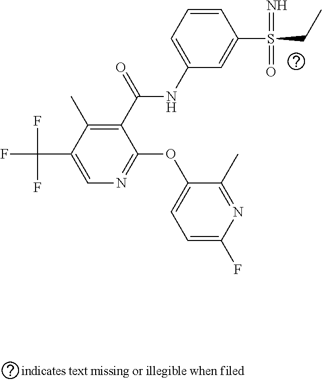 [SMILES: CC[S@@](=N)(=O)c1cccc(NC(=O)c2c(Oc3ccc(F)nc3C)ncc(C(F)(F)F)c2C)c1] | 497.1 | ¹H NMR (400 MHz, DMSO-d₆) δ 11.19 (s, 1 H), 8.55 (s, 1 H), 8.35 (d, J = 1.7 Hz, 1 H), 7.94-7.81 (m, 2 H), 7.68-7.58 (m, 2 H), 7.11 (dd, J = 8.7, 3.4 Hz, 1 H), 4.22 (s, 1 H), 3.14-3.11 (m, 2 H), 2.50 (s, 3 H), 2.24 (s, 3 H), 1.11-1.07 (m, 3 H). |
| 83C | 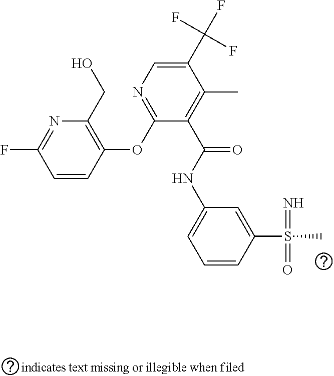 [SMILES: Cc1c(C(F)(F)F)cnc(Oc2ccc(F)nc2CO)c1C(=O)Nc1cccc([S@@](C)(=N)=O)c1] | 499.0 | ¹H NMR (400 MHz, DMSO-d₆, ppm) δ 11.14 (s, 1 H), 8.53 (s, 1 H), 8.41 (s, 1 H), 7.94-7.84 (m, 2 H), 7.73- 7.59 (m, 1 H), 7.21 (dd, J = 8.7, 3.5 Hz, 1 H), 5.36-5.12 (m, 1 H), 4.39 (s, 2 H), 3.08 (s, 3 H), 2.50-2.44 (m, 3 H) |
| 84C | 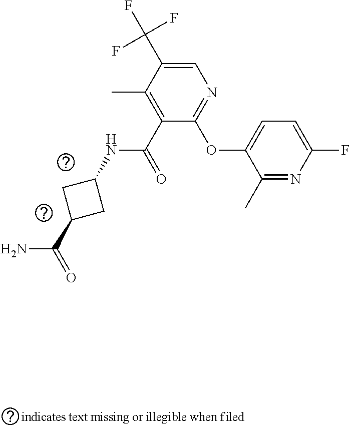 [SMILES: Cc1nc(F)ccc1Oc1ncc(C(F)(F)F)c(C)c1C(=O)N[C@H]1C[C@H](C(N)=O)C1] | 427.0 | ¹H NMR (400 MHz, DMSO-d₆, ppm) δ 9.06 (d, J = 7.3 Hz, 1 H), 8.46 (s, 1 H), 7.81 (dd, J = 8.6, 7.0 Hz, 1 H), 7.25 (s, 1 H), 7.11 (dd, J = 8.7, 3.4 Hz, 1 H), 6.80 (s, 1 H), 4.55-4.44 (m, 1 H), 2.96-2.87 (m, 1 H), 2.48-2.42 (m, 2 H), 2.41 (s, 3 H), 2.25 (s, 3 H), 2.17-2.10 (m, 2 H). |
| 85C | 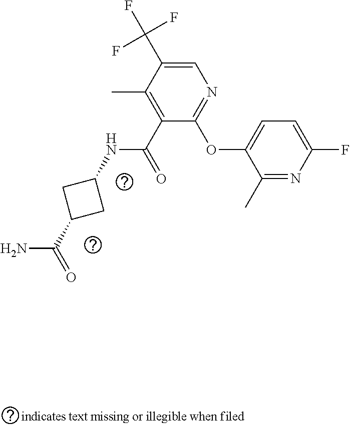 [SMILES: Cc1nc(F)ccc1Oc1ncc(C(F)(F)F)c(C)c1C(=O)N[C@H]1C[C@@H](C(N)=O)C1] | 427.1 | ¹H NMR (400 MHz, DMSO-d₆, ppm) δ 9.01 (d, J = 7.5 Hz, 1 H), 8.44 (s, 1 H), 7.80 (dd, J = 8.6, 7.0 Hz, 1 H), 7.23 (s, 1 H), 7.10 (dd, J = 8.6, 3.4 Hz, 1 H), 6.75 (s, 1 H), 4.34-4.28 (m, 1 H), 2.71-2.67 (m, 1 H), 2.44-2.36 (m, 5 H), 2.23 (s, 3 H), 2.12-2.03 (m, 2 H). |
| 86C | 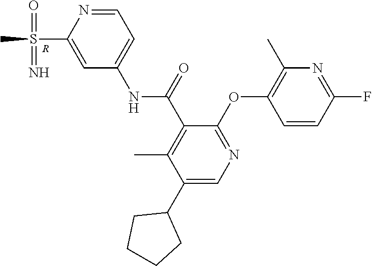 | 484.1 | ¹H NMR (400 MHz, DMSO-d₆, ppm) δ 11.50 (s, 1 H), 8.63 (d, J = 5.4 Hz, 1 H), 8.50 (d, J = 1.8 Hz, 1 H), 8.07 (s, 1 H), 7.75-7.72 (m, 2 H), 7.06 (dd, J = 8.6, 3.4 Hz, 1 H), 4.39 (s, 1 H), 3.21-3.11 (m, 4 H), 2.36 (s, 3 H), 2.23 (s, 3 H), 2.06-1.95 (m, 2 H), 1.84-1.73 (m, 2 H), 1.71-1.50 (m, 4 H). |
| 87C | 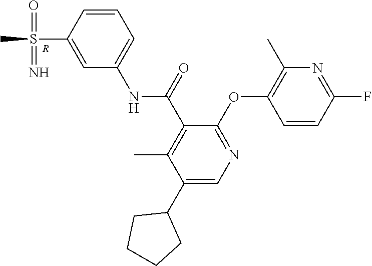 | 483.1 | ¹H NMR (400 MHz, DMSO-d₆, ppm) δ 11.04 (s, 1 H), 8.45 (s, 1 H), 8.04 (s, 1 H), 7.88 (d, J = 8.1 Hz, 1 H), 7.75 (dd, J = 8.5, 7.1 Hz, 1 H), 7.67 (d, J = 7.9 Hz, 1 H), 7.59 (t, J = 7.9 Hz, 1 H), 7.05 (dd, J = 8.6, 3.4 Hz, 1 H), 4.22 (s, 1 H), 3.21-3.11 (m, 1 H), 3.06 (s, 3 H), 2.36 (s, 3 H), 2.23 (s, 3 H), 2.05-1.95 (m, 2 H), 1.85-1.75 (m, 2 H), 1.72-1.62 (m, 2 H), 1.61-1.51 (m, 2 H). |
| 88C | 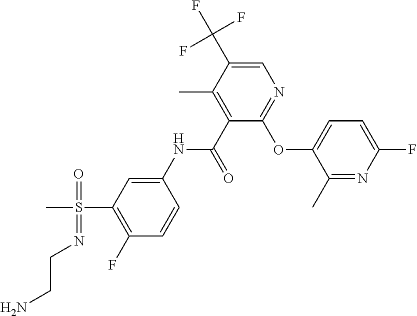 [SMILES: Cc1nc(F)ccc1Oc1ncc(C(F)(F)F)c(C)c1C(=O)Nc1ccc(F)c(S(C)(=O)=NCCN)c1] | 544.1 | ¹H NMR (400 MHz, DMSO-d₆, ppm) δ 11.49 (s, 1 H), 8.54 (s, 1 H), 8.34 (dd, J = 7.3, 4.8 Hz, 2 H), 8.05-7.95 (m, 1 H), 7.89-7.80 (m, 1 H), 7.51 (t, J = 9.3 Hz, 1 H), 7.12 (dd, J = 8.6, 3.1 Hz, 1 H), 3.32 (s, 3 H), 3.05-2.98 (m, 1 H), 2.87-2.81 (m, 1 H), 2.74 (t, J = 6.2 Hz, 2 H), 2.47 (s, 3 H), 2.24 (s, 3 H). |
| 89C | 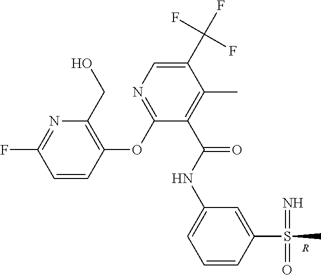 | 499.1 | ¹H NMR (400 MHz, DMSO-d₆) δ 11.15 (s, 1 H), 8.54 (s, 1 H), 8.43 (s, 1 H), 7.94-7.84 (m, 2 H), 7.75-7.58 (m, 2 H), 7.21 (dd, J = 8.7, 3.5 Hz, 1 H), 5.24 (s, 1 H), 4.40 (s, 2 H), 3.10 (s, 3 H), 2.50 (s, 3 H). ¹H NMR (400 MHz, CD₃OD) δ 8.52-8.37 (m, 2 H), 8.02-7.94 (m, 1 H), 7.85-7.77 (m, 2 H), 7.65 (t, J = 8.0 Hz, 1 H), 7.07 (dd, J = 8.7, 3.3 Hz, 1 H), 4.55 (s, 2 H), 3.18 (s, 3 H), 2.58 (s, 3 H). |
| 90C | 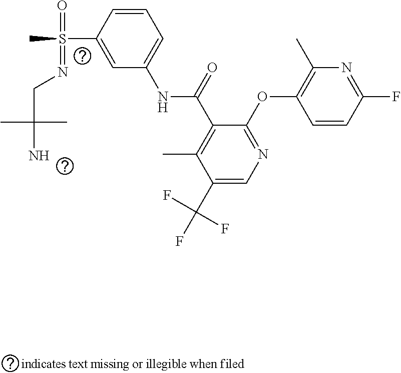 [SMILES: Cc1nc(F)ccc1Oc1ncc(C(F)(F)F)c(C)c1C(=O)Nc1cccc([S@@](C)(=O)=NCC(C)(C)N)c1] | 554.2 | ¹H NMR (400 MHz, DMSO-d₆, ppm, formate salt) δ 11.40 (s, 1 H), 8.55 (s, 1 H), 8.40 (s, 1 H), 8.34 (s, 1 H), 7.93-7.79 (m, 2 H), 7.69-7.62 (m, 2 H), 7.12 (dd, J = 8.6, 3.3 Hz, 1 H), 3.23 (s, 3 H), 2.87-2.80 (m, 1 H), 2.68-2.60 (m, 1 H), 2.51 (s, 3 H), 2.24 (s, 3 H), 1.16 (d, J = 4.6 Hz, 6 H). |
| 91C | 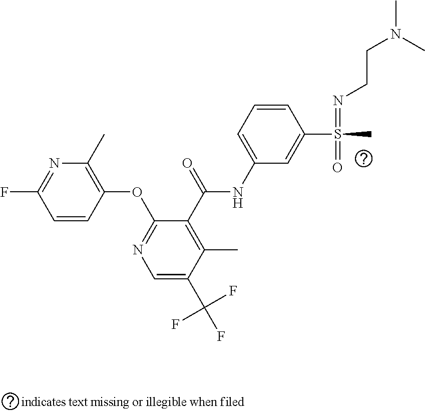 [SMILES: Cc1nc(F)ccc1Oc1ncc(C(F)(F)F)c(C)c1C(=O)Nc1cccc([S@@](C)(=O)=NCCN(C)C)c1] | 554.1 | ¹H NMR (400 MHz, DMSO-d₆, ppm, TFA salt) δ 11.25 (s, 1 H), 8.55 (s, 1 H), 8.36 (s, 1 H), 8.22 (s, 1 H), 7.94- 7.80 (m, 2 H), 7.71-7.55 (m, 2 H), 7.12 (dd, J = 8.6, 3.1 Hz, 1 H), 3.14 (s, 3 H), 2.97-2.88 (m, 1 H), 2.83- 2.74 (m, 1 H), 2.50 (s, 3 H), 2.43 (t, J = 7.0 Hz, 2 H), 2.24 (s, 3 H), 2.14 (s, 6 H). |
| 92C | 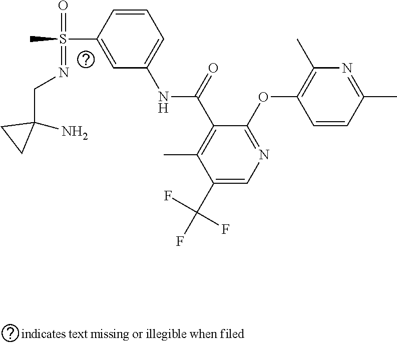 [SMILES: Cc1ccc(Oc2ncc(C(F)(F)F)c(C)c2C(=O)Nc2cccc([S@@](C)(=O)=NCC3(N)CC3)c2)c(C)n1] | 552.1 | ¹H NMR (400 MHz, DMSO-d₆, ppm, TFA salt) δ 11.26 (s, 1 H), 8.56 (s, 1 H), 8.43 (s, 1 H), 8.07 (s, 3 H), 7.84 (dd, J = 8.8, 7.0 Hz, 2 H), 7.74-7.61 (m, 2 H), 7.12 (dd, J = 8.8, 3.3 Hz, 1 H), 3.24 (s, 3 H), 3.07-3.01 (m, 1 H), 2.84-2.79 (m, 1 H), 2.51 (s, 3 H), 2.25 (s, 3 H), 0.85-0.68 (m, 4 H). |
| 93C | 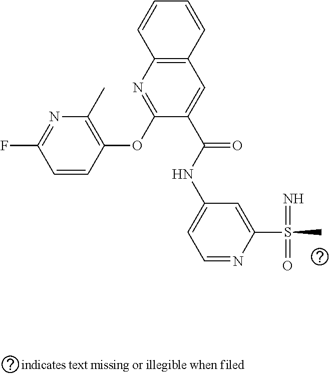 [SMILES: Cc1nc(F)ccc1Oc1nc2ccccc2cc1C(=O)Nc1ccnc([S@](C)(=N)=O)c1] | 452.0 | ¹H NMR (400 MHz, DMSO-d₆, ppm) δ 11.41 (s, 1 H), 8.83 (s, 1 H), 8.66 (d, J = 5.5 Hz, 1 H), 8.50 (d, J = 1.8 Hz, 1 H), 8.14-7.94 (m, 1 H), 8.18- 7.89 (m, 2 H), 7.81-7.56 (m, 3 H), 7.18-7.12 (m, 1 H), 4.39 (s, 1 H), 3.16 (s, 3 H), 2.28 (s, 3 H). |
| 94C | 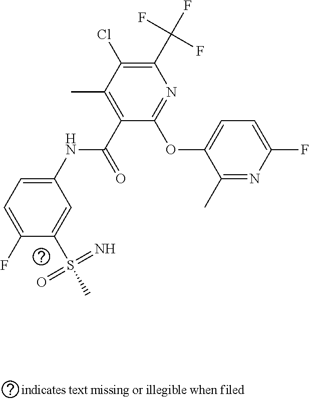 [SMILES: Cc1nc(F)ccc1Oc1nc(C(F)(F)F)c(Cl)c(C)c1C(=O)Nc1ccc(F)c([S@@](C)(=N)=O)c1] | 535.0 | ¹H NMR (400 MHz, DMSO-d₆, ppm) δ 11.21 (s, 1 H), 8.31 (dd, J = 6.4, 2.7 Hz, 1 H), 7.97-7.81 (m, 2 H), 7.51- 7.45 (m, 1 H), 7.14-7.09 (m, 1 H), 4.74 (s, 1 H), 3.20 (s, 3 H), 2.51 (s, 3 H), 2.25 (s, 3 H). |
| 95C | 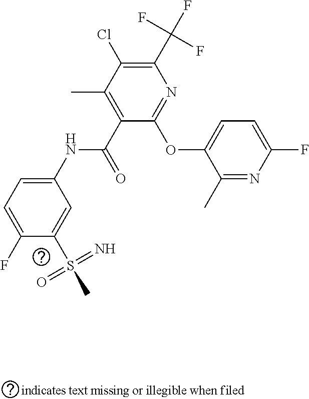 [SMILES: Cc1nc(F)ccc1Oc1nc(C(F)(F)F)c(Cl)c(C)c1C(=O)Nc1ccc(F)c([S@](C)(=N)=O)c1] | 535.0 | ¹H NMR (400 MHz, DMSO-d₆, ppm) δ 11.27 (s, 1 H), 8.31 (dd, J = 6.4, 2.7 Hz, 1 H), 7.97-7.85 (m, 2 H), 7.51- 7.45 (m, 1 H), 7.14-7.10 (m, 1 H), 4.73 (s, 1 H), 3.20 (s, 3 H), 2.51 (s, 3 H), 2.25 (s, 3 H). |
| 96C | 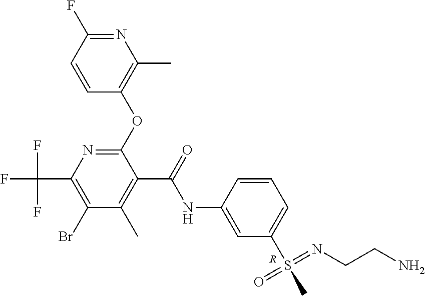 | 605.9 | ¹H NMR (400 MHz, DMSO-d₆, ppm, TFA salt) δ 11.32 (s, 1 H), 8.40 (s, 1 H), 7.92-7.83 (m, 2 H), 7.77-7.62 (m, 5 H), 7.12 (dd, J = 8.7, 3.3 Hz, 1 H), 3.23 (s, 3 H), 3.08-2.84 (m, 4 H), 2.55 (s, 3 H), 2.26 (s, 3 H). |
| 97C | 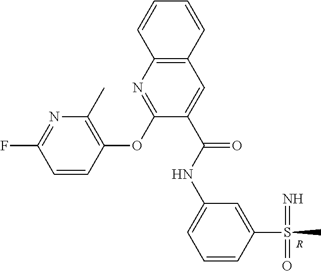 | 451.1 | ¹H NMR (400 MHz, DMSO-d₆, ppm) δ 11.01 (s, 1 H), 8.79 (s, 1 H), 8.45 (s, 1 H), 8.09 (d, J = 7.4 Hz, 1 H), 8.01-7.93 (m, 2 H), 7.78-7.56 (m, 5 H), 7.15 (dd, J = 8.6, 3.3 Hz, 1 H), 4.24 (s, 1 H), 3.08 (s, 3 H), 2.27 (s, 3 H). |
| 98C | 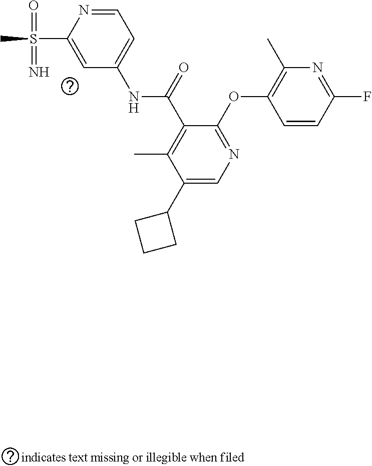 [SMILES: Cc1nc(F)ccc1Oc1ncc(C2CCC2)c(C)c1C(=O)Nc1ccnc([S@](C)(=N)=O)c1] | 470.1 | ¹H NMR (400 MHz, DMSO-d₆, ppm) δ 11.47 (s, 1 H), 8.61 (d, J = 4.9 Hz, 1 H), 8.48 (s, 1 H), 8.01 (s, 1 H), 7.81-7.72 (m, 2 H), 7.06 (dd, J = 8.7, 3.4 Hz, 1 H), 4.37 (s, 1 H), 3.63 (t, J = 8.8 Hz, 1 H), 3.14 (s, 3 H), 2.36- 2.30 (m, 2 H), 2.25 (s, 3 H), 2.22 (s, 3 H), 2.15-2.07 (m, 2 H), 2.04-1.99 (m, 1 H), 1.81-1.78 (m, 1 H). |
| 99C | 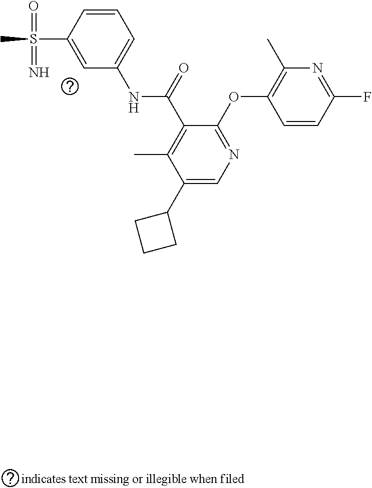 [SMILES: Cc1nc(F)ccc1Oc1ncc(C2CCC2)c(C)c1C(=O)Nc1cccc([S@](C)(=N)=O)c1] | 469.1 | ¹H NMR (400 MHz, DMSO-d₆, ppm) δ 11.02 (s, 1 H), 8.43 (t, J = 1.8 Hz, 1 H), 7.99 (s, 1 H), 7.88 (d, J = 8.9 Hz, 1 H), 7.75 (dd, J = 8.6, 7.0 Hz, 1 H), 7.67 (d, J = 8.0 Hz, 1 H), 7.59 (t, J = 7.9 Hz, 1 H), 7.06 (dd, J = 8.6, 3.4 Hz, 1 H), 4.21 (s, 1 H), 3.70-3.57 (m, 1 H), 3.06 (s, 3 H), 2.36-2.29 (m, 2 H), 2.25 (s, 3 H), 2.23 (s, 3 H), 2.18- 2.08 (m, 2 H), 2.06-1.96 (m, 1 H), 1.83-1.76 (m, 1 H). |
| 100C | 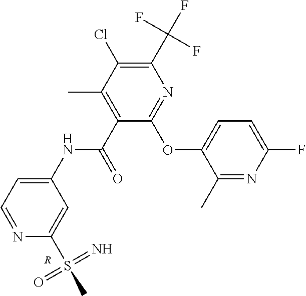 | 518.0 | ¹H NMR (400 MHz, DMSO-d₆, ppm) δ 11.78 (s, 1 H), 8.73 (d, J = 5.4 Hz, 1 H), 8.53 (d, J = 1.7 Hz, 1 H), 7.89 (dd, J = 8.6, 7.0 Hz, 1 H), 7.84 (dd, J = 5.4, 1.9 Hz, 1 H), 7.13 (dd, J = 8.7, 3.4 Hz, 1 H), 4.27 (s, 1 H), 3.39 (s, 3 H), 2.52 (s, 3 H), 2.25 (s, 3 H). |
| 101C | 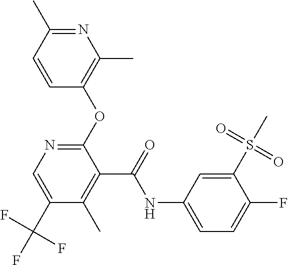 [SMILES: Cc1ccc(Oc2ncc(C(F)(F)F)c(C)c2C(=O)Nc2ccc(F)c(S(C)(=O)=O)c2)c(C)n1] | 502.1 | ¹H NMR (400 MHz, DMSO-d₆, ppm) δ 11.29 (s, 1 H), 8.56 (s, 1 H), 8.39 (dd, J = 6.2, 2.7 Hz, 1 H), 8.04-7.94 (m, 1 H), 7.84 (dd, J = 8.6, 7.0 Hz, 1 H), 7.57 (t, J = 9.4 Hz, 1 H), 7.12 (dd, J = 8.7, 3.3 Hz, 1 H), 3.36 (s, 3 H), 2.50 (s, 3 H), 2.24 (s, 3 H). |
| 102C | 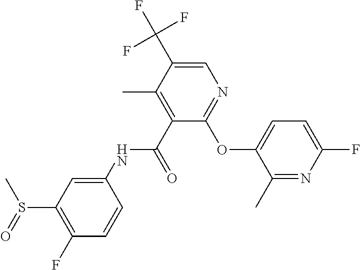 [SMILES: Cc1nc(F)ccc1Oc1ncc(C(F)(F)F)c(C)c1C(=O)Nc1ccc(F)c(S(C)=O)c1] | 486.0 | ¹H NMR (400 MHz, DMSO-d₆, ppm) δ 11.20 (s, 1 H), 8.55 (s, 1 H), 8.20 (dd, J = 6.0, 2.7 Hz, 1 H), 7.90-7.78 (m, 2 H), 7.42 (t, J = 9.1 Hz, 1 H), 7.11 (dd, J = 8.7, 3.3 Hz, 1 H), 2.84 (s, 3 H), 2.47 (m, 3 H), 2.24 (s, 3 H). |
| 103C | 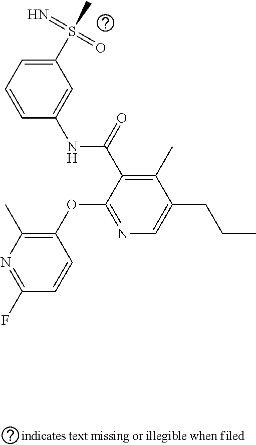 [SMILES: CCCc1cnc(Oc2ccc(F)nc2C)c(C(=O)Nc2cccc([S@](C)(=N)=O)c2)c1C] | 457.0 | ¹H NMR (400 MHz, DMSO-d₆, ppm) δ 11.03 (s, 1 H), 8.44 (t, J = 1.8 Hz, 1 H), 7.95-7.86 (m, 2 H), 7.79-7.55 (m, 3 H), 7.06 (dd, J = 8.6, 3.4 Hz, 1 H), 4.39 (s, 1 H), 3.08 (s, 3 H), 2.62-2.55 (m, 2 H), 2.51 (s, 3 H), 2.23 (s, 3 H), 1.61-1.45 (m, 2 H), 0.95 (t, J = 7.3 Hz, 3 H). |
| 104C | 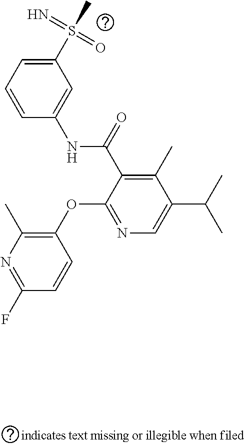 [SMILES: Cc1nc(F)ccc1Oc1ncc(C(C)C)c(C)c1C(=O)Nc1cccc([S@](C)(=N)=O)c1] | 457.1 | ¹H NMR (400 MHz, DMSO-d₆, ppm) δ 11.05 (s, 1 H), 8.45 (s, 1 H), 8.06 (s, 1 H), 7.92-7.63 (m, 3 H), 7.59 (t, J = 7.9 Hz, 1 H), 7.06 (dd, J = 8.6, 3.4 Hz, 1 H), 4.22 (s, 1 H), 3.18-3.09 (m, 1 H), 3.06 (s, 3 H), 2.35 (s, 3 H), 2.24 (s, 3 H), 1.23 (d, J = 6.8 Hz, 6 H). |
| 105C | 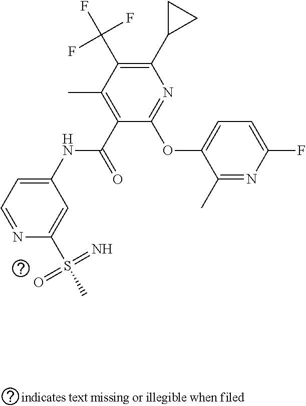 [SMILES: Cc1nc(F)ccc1Oc1nc(C2CC2)c(C(F)(F)F)c(C)c1C(=O)Nc1ccnc([S@@](C)(=N)=O)c1] | 524.1 | ¹H NMR (400 MHz, DMSO-d₆, ppm) δ 11.52 (s, 1 H), 8.64 (d, J = 5.4 Hz, 1 H), 8.46 (d, J = 1.9 Hz, 1 H), 7.84- 7.75 (m, 2 H), 7.11 (dd, J = 8.7, 3.4 Hz, 1 H), 4.40 (s, 1 H), 3.15 (s, 3 H), 2.49-2.45 (m, 4 H), 2.20 (s, 3 H), 0.92 (dd, J = 7.7, 3.1 Hz, 2 H), 0.61 (d, J = 3.2 Hz, 2 H). |
| 106C | 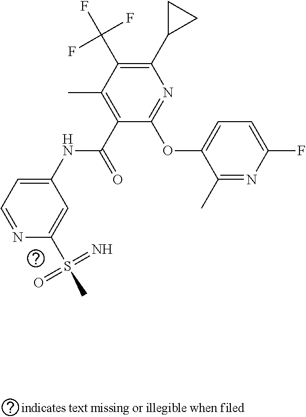 [SMILES: Cc1nc(F)ccc1Oc1nc(C2CC2)c(C(F)(F)F)c(C)c1C(=O)Nc1ccnc([S@](C)(=N)=O)c1] | 524.1 | ¹H NMR (400 MHz, DMSO-d₆, ppm) δ 11.52 (s, 1 H), 8.64 (d, J = 5.4 Hz, 1 H), 8.46 (d, J = 1.8 Hz, 1 H), 7.84- 7.74 (m, 2 H), 7.11 (dd, J = 8.7, 3.4 Hz, 1 H), 4.40 (s, 1 H), 3.15 (s, 3 H), 2.47 (dd, J = 6.2, 3.1 Hz, 4 H), 2.20 (s, 3 H), 0.92 (dd, J = 7.6, 3.2 Hz, 2 H), 0.61 (d, J = 3.2 Hz, 2 H). |
| 107C | 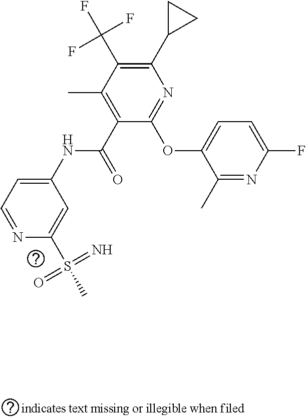 [SMILES: Cc1nc(F)ccc1Oc1nc(C2CC2)c(C(F)(F)F)c(C)c1C(=O)Nc1ccnc([S@@](C)(=N)=O)c1] | 523.1 | ¹H NMR (400 MHz, DMSO-d₆, ppm) δ 11.09 (s, 1 H), 8.41 (s, 1 H), 7.88- 7.75 (m, 2 H), 7.71-7.56 (m, 2 H), 7.11 (dd, J = 8.7, 3.4 Hz, 1 H), 4.24 (s, 1 H), 3.06 (s, 3 H), 2.49-2.45 (m, 3 H), 2.27 (d, J = 2.3 Hz, 1 H), 2.21 (s, 3 H), 0.91 (dd, J = 7.7, 3.2 Hz, 2 H), 0.61 (d, J = 3.3 Hz, 2 H). |
| 108 | 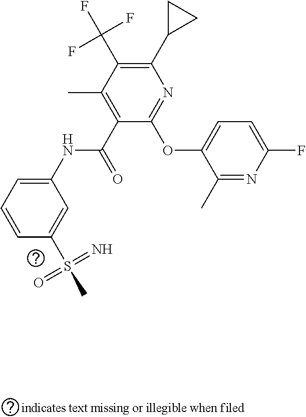 [SMILES: Cc1nc(F)ccc1Oc1nc(C2CC2)c(C(F)(F)F)c(C)c1C(=O)Nc1cccc([S@](C)(=N)=O)c1] | 523.1 | ¹H NMR (400 MHz, DMSO-d₆, ppm) δ 11.09 (s, 1 H), 8.41 (s, 1 H), 7.86 (d, J = 8.1 Hz, 1 H), 7.79 (dd, J = 8.5, 7.1 Hz, 1 H), 7.69 (d, J = 7.8 Hz, 1 H), 7.60 (t, J = 7.9 Hz, 1 H), 7.11 (dd, J = 8.6, 3.4 Hz, 1 H), 4.24 (s, 1 H), 3.06 (s, 3 H), 2.48-2.46 (m, 3 H), 2.30-2.23 (m, 1 H), 2.21 (s, 3 H), 0.95-0.85 (m, 2 H), 0.65-0.56 (m, 2 H) |
| 109 | 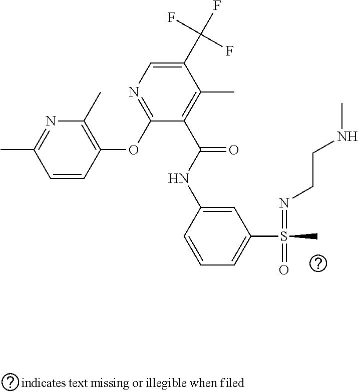 | 539.9 | ¹H NMR (400 MHz, DMSO-d₆, ppm) δ 11.23 (s, 1 H), 8.55 (s, 1 H), 8.34 (s, 1 H), 7.93-7.82 (m, 2 H), 7.64 (q, J = 7.7 Hz, 2 H), 7.12 (dd, J = 8.7, 3.2 Hz, 1 H), 3.14 (s, 3 H), 2.89-2.73 (m, 2 H), 2.53 (s, 1 H), 2.51 (s, 2 H), 2.51 (s, 3 H), 2.24 (s, 3 H), 2.21 (s, 3 H). |
| 110C | 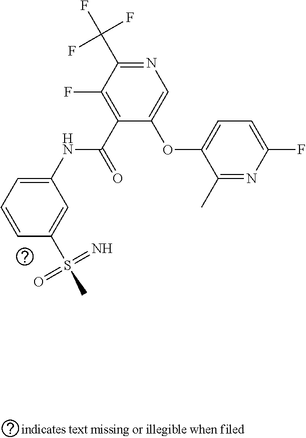 [SMILES: Cc1nc(F)ccc1Oc1cnc(C(F)(F)F)c(F)c1C(=O)Nc1cccc([S@](C)(=N)=O)c1] | 486.9 | ¹H NMR (400 MHz, DMSO-d₆, ppm) δ 11.40 (s, 1 H), 8.33 (s, 1 H), 8.26 (s, 1 H), 7.94-7.79 (m, 2 H), 7.76- 7.58 (m, 2 H), 7.13 (dd, J = 8.7, 3.2 Hz, 1 H), 4.27 (s, 1 H), 3.07 (s, 3 H), 2.34 (s, 3 H). |
| 111C | 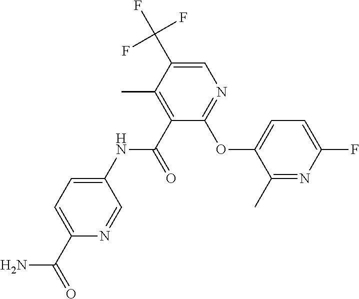 [SMILES: Cc1nc(F)ccc1Oc1ncc(C(F)(F)F)c(C)c1C(=O)Nc1ccc(C(N)=O)nc1] | 450.0 | ¹H NMR (400 MHz, DMSO-d₆, ppm) δ 11.37 (s, 1 H), 8.89 (d, J = 2.3 Hz, 1 H), 8.57 (s, 1 H), 8.35 (dd, J = 8.6, 2.5 Hz, 1 H), 8.09 (d, J = 8.5 Hz, 1 H), 8.03 (s, 1 H), 7.86 (dd, J = 8.6, 7.0 Hz, 1 H), 7.58 (s, 1 H), 7.12 (dd, J = 8.7, 3.4 Hz, 1 H), 2.51 (s, 3 H), 2.25 (s, 3 H). |
| 112C | 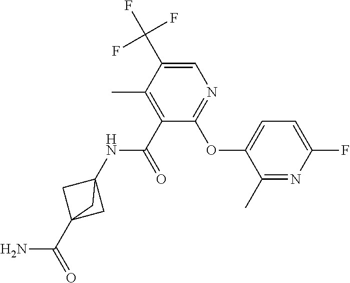 [SMILES: Cc1nc(F)ccc1Oc1ncc(C(F)(F)F)c(C)c1C(=O)NC12CC(C(N)=O)(C1)C2] | 439.0 | ¹H NMR (400 MHz, DMSO-d₆, ppm) δ 9.28 (s, 1 H), 8.45 (s, 1 H), 7.88- 7.76 (m, 1 H), 7.28 (s, 1 H), 7.10 (dd, J = 8.6, 3.2 Hz, 1 H), 6.99 (s, 1 H), 2.48 (s, 3 H), 2.40 (s, 3 H), 2.25 (s, 2 H), 2.23 (s, 4 H). |
| 113C | 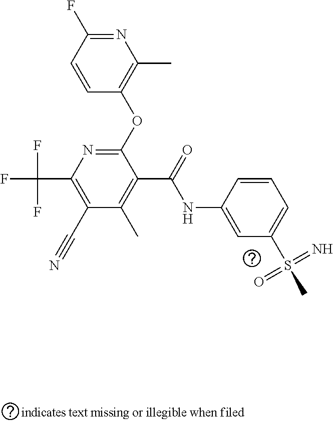 [SMILES: Cc1nc(F)ccc1Oc1nc(C(F)(F)F)c(C#N)c(C)c1C(=O)Nc1cccc([S@](C)(=N)=O)c1] | 508.0 | ¹H NMR (400 MHz, DMSO-d₆, ppm) δ 11.27 (s, 1 H), 8.38 (s, 1 H), 7.95- 7.83 (m, 2 H), 7.77-7.60 (m, 2 H), 7.16 (dd, J = 8.7, 3.3 Hz, 1 H), 4.26 (s, 1 H), 3.08 (s, 3 H), 2.67 (s, 3 H), 2.26 (s, 3 H). |
| 114C | 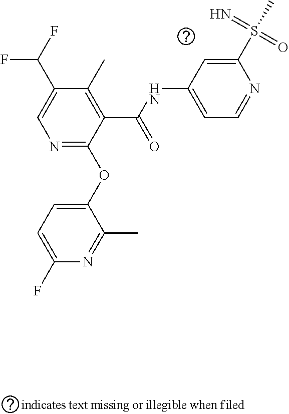 [SMILES: Cc1nc(F)ccc1Oc1ncc(C(F)F)c(C)c1C(=O)Nc1ccnc([S@@](C)(=N)=O)c1] | 466.0 | ¹H NMR (400 MHz, DMSO-d₆, ppm) δ 11.59 (s, 1 H), 8.64 (d, J = 5.4 Hz, 1 H), 8.47 (d, J = 1.2 Hz, 1 H), 8.35 (s, 1 H), 7.86-7.76 (m, 2 H), 7.43- 7.06 (m, 2 H), 4.40 (s, 1 H), 3.15 (s, 3 H), 2.46 (s, 3 H), 2.23 (s, 3 H). |
| 115C | 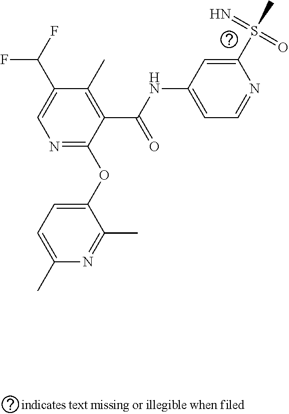 [SMILES: Cc1ccc(Oc2ncc(C(F)F)c(C)c2C(=O)Nc2ccnc([S@](C)(=N)=O)c2)c(C)n1] | 466.0 | ¹H NMR (400 MHz, DMSO-d₆, ppm) δ 11.59 (s, 1 H), 8.65 (d, J = 5.4 Hz, 1 H), 8.47 (d, J = 1.8 Hz, 1 H), 8.35 (s, 1 H), 7.86-7.76 (m, 2 H), 7.43- 7.05 (m, 2 H), 4.39 (s, 1 H), 3.15 (s, 3 H), 2.47 (s, 3 H), 2.23 (s, 3 H). |
| 116C | 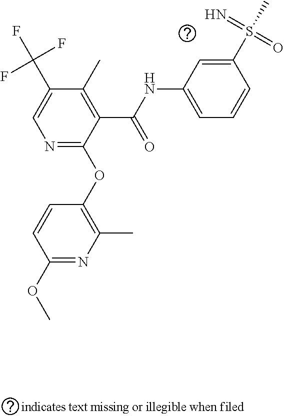 [SMILES: COc1ccc(Oc2ncc(C(F)(F)F)c(C)c2C(=O)Nc2cccc([S@@](C)(=N)=O)c2)c(C)n1] | 495.1 | ¹H NMR (400 MHz, DMSO-d₆, ppm) δ 11.18 (s, 1 H), 8.44 (m, J = 23.7, 21.9 Hz, 2 H), 7.89 (dd, J = 8.1, 0.9 Hz, 1 H), 7.70 (d, J = 7.9 Hz, 1 H), 7.65-7.45 (m, 2 H), 6.72 (d, J = 8.7 Hz, 1 H), 4.25 (s, 1 H), 3.83 (s, 3 H), 3.07 (s, 3 H), 2.49 (s, 3 H), 2.21 (s, 3 H). |
| 117C | 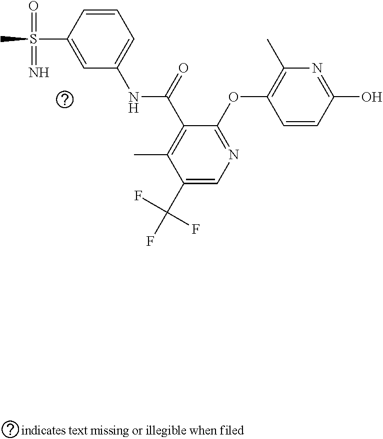 [SMILES: Cc1nc(O)ccc1Oc1ncc(C(F)(F)F)c(C)c1C(=O)Nc1cccc([S@](C)(=N)=O)c1] | 481.1 | ¹H NMR (400 MHz, DMSO-d₆, ppm) δ 11.66 (s, 1 H), 11.14 (s, 1 H), 8.56 (s, 1 H), 8.39 (s, 1 H), 7.87 (d, J = 8.1 Hz, 1 H), 7.70 (d, J = 7.9 Hz, 1 H), 7.61 (t, J = 7.9 Hz, 1 H), 7.34 (d, J = 9.7 Hz, 1 H), 6.22 (d, J = 9.7 Hz, 1 H), 4.25 (s, 1 H), 3.07 (s, 3 H), 2.46 (s, 3 H), 2.02 (s, 3 H). |
| 118C | 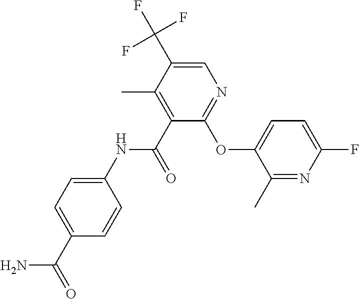 [SMILES: Cc1nc(F)ccc1Oc1ncc(C(F)(F)F)c(C)c1C(=O)Nc1ccc(C(N)=O)cc1] | 449.0 | ¹H NMR (400 MHz, DMSO-d₆, ppm) δ 11.06 (s, 1 H), 8.54 (s, 1 H), 8.00- 7.73 (m, 6 H), 7.30 (s, 1 H), 7.11 (d, J = 8.7 Hz, 1 H), 2.50 (s, 3 H), 2.25 (s, 3 H). |
| 119C | 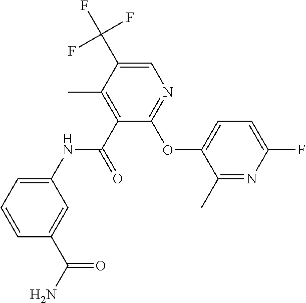 [SMILES: Cc1nc(F)ccc1Oc1ncc(C(F)(F)F)c(C)c1C(=O)Nc1cccc(C(N)=O)c1] | 449.1 | ¹H NMR (400 MHz, DMSO-d₆, ppm) δ 10.97 (s, 1 H), 8.54 (s, 1 H), 8.21 (s, 1 H), 8.00 (s, 1 H), 7.89-7.78 (m, 2 H), 7.63 (d, J = 7.8 Hz, 1 H), 7.44 (dd, J = 18.1, 10.2 Hz, 2 H), 7.11 (dd, J = 8.6, 3.4 Hz, 1 H), 2.50 (s, 3 H), 2.25 (s, 3 H). |
| 120C | 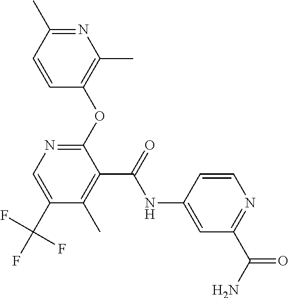 [SMILES: Cc1ccc(Oc2ncc(C(F)(F)F)c(C)c2C(=O)Nc2ccnc(C(N)=O)c2)c(C)n1] | 450.1 | ¹H NMR (400 MHz, DMSO-d₆, ppm) δ 11.45 (s, 1 H), 8.58 (d, J = 5.5 Hz, 2 H), 8.37 (d, J = 2.0 Hz, 1 H), 8.13 (d, J = 2.2 Hz, 1 H), 7.90-7.81 (m, 2 H), 7.69 (d, J = 2.2 Hz, 1 H), 7.12 (dd, J = 8.7, 3.4 Hz, 1 H), 2.50 (s, 3 H), 2.24 (s, 3 H). |
| 121C | 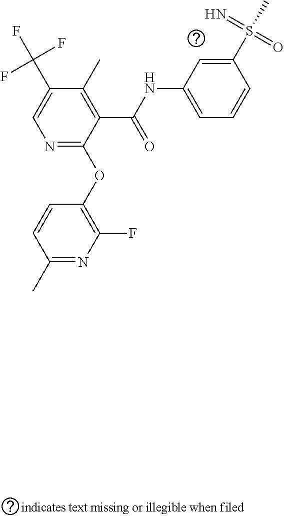 [SMILES: Cc1ccc(Oc2ncc(C(F)(F)F)c(C)c2C(=O)Nc2cccc([S@@](C)(=N)=O)c2)c(F)n1] | 482.8 | ¹H NMR (400 MHz, DMSO-d₆, ppm) δ 11.20 (s, 1 H), 8.56 (s, 1 H), 8.40 (t, J = 1.8 Hz, 1 H), 7.92-7.60 (m, 4 H), 7.30 (d, J = 8.0 Hz, 1 H), 4.24 (s, 1 H), 3.07 (s, 3 H), 2.49 (s, 3H), 2.45 (s, 3 H). |
| 122C | 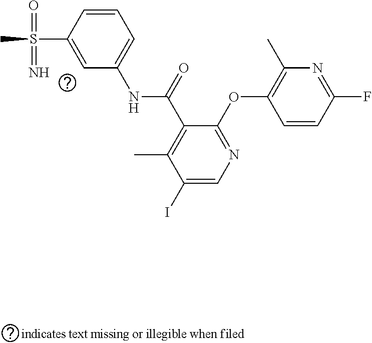 [SMILES: Cc1nc(F)ccc1Oc1ncc(I)c(C)c1C(=O)Nc1cccc([S@](C)(=N)=O)c1] | 541.0 | ¹H NMR (400 MHz, DMSO-d₆, ppm) δ 11.09 (s, 1 H), 8.49 (s, 1 H), 8.41 (t, J = 1.8 Hz, 1 H), 7.87 (d, J = 8.0 Hz, 1 H), 7.78 (dd, J = 8.6, 7.0 Hz, 1 H), 7.69 (d, J = 8.0 Hz, 1 H), 7.60 (t, J = 7.9 Hz, 1 H), 7.08 (dd, J = 8.7, 3.4 Hz, 1 H), 4.23 (s, 1 H), 3.06 (s, 3 H), 2.44 (s, 3 H), 2.23 (s, 3 H). |
| 123C | 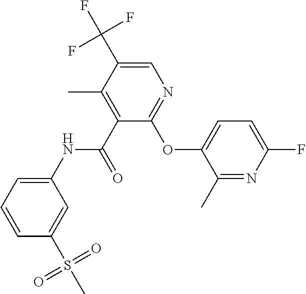 [SMILES: Cc1nc(F)ccc1Oc1ncc(C(F)(F)F)c(C)c1C(=O)Nc1cccc(S(C)(=O)=O)c1] | 484.0 | ¹H NMR (400 MHz, DMSO-d₆, ppm) δ 11.28 (s, 1 H), 8.56 (s, 1 H), 8.43 (d, J = 1.7 Hz, 1 H), 7.94-7.89 (m, 1 H), 7.84 (dd, J = 8.6, 7.0 Hz, 1 H), 7.75-7.65 (m, 2 H), 7.12 (dd, J = 8.7, 3.3 Hz, 1 H), 3.24 (s, 3 H), 2.50 (s, 3 H), 2.24 (s, 3 H). |
| 124C | 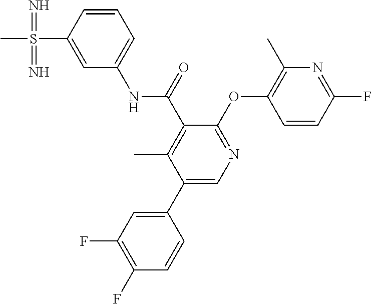 [SMILES: Cc1nc(F)ccc1Oc1ncc(-c2ccc(F)c(F)c2)c(C)c1C(=O)Nc1cccc(S(C)(=N)=N)c1] | 526.1 | ¹H NMR (400 MHz, DMSO-d₆, ppm) δ 11.11 (s, 1 H), 8.54 (s, 1 H), 8.05 (s, 1 H), 7.88-7.86 (m, 1 H), 7.83-7.78 (m, 2 H), 7.60-7.52 (m, 3 H), 7.25- 7.23 (m, 1 H), 7.11-7.07 (m, 1 H), 3.01 (s, 3 H), 2.91 (s, 2 H), 2.30 (s, 3 H), 2.28 (s, 3 H). |
| 125C | 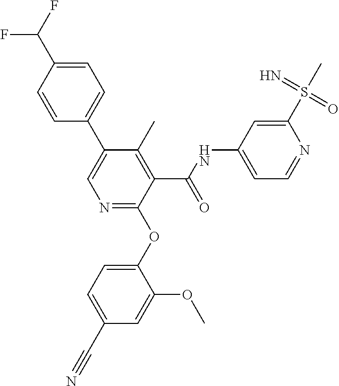 [SMILES: COc1cc(C#N)ccc1Oc1ncc(-c2ccc(C(F)F)cc2)c(C)c1C(=O)Nc1ccnc(S(C)(=N)=O)c1] | 564.1 | ¹H NMR (400 MHz, DMSO-d₆, 353 K, ppm) δ 11.62 (bs, 1 H), 8.58 (d, J = 5.2 Hz, 1 H), 8.02 (s, 1 H), 7.63- 7.58 (m, 4 H), 7.43 (d, J = 6.8 Hz, 1 H), 7.35-7.33 (m, 3 H), 7.17 (s, 1 H), 7.14-6.86 (m, 1 H), 4.18 (s, 1 H), 3.77 (s, 3 H), 3.12 (s, 3 H), 2.07 (s, 3 H) |
| 126C | 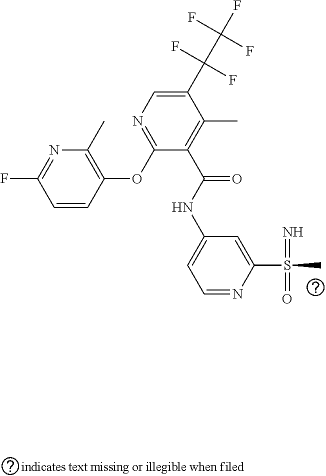 [SMILES: Cc1nc(F)ccc1Oc1ncc(C(F)(F)C(F)(F)F)c(C)c1C(=O)Nc1ccnc([S@](C)(=N)=O)c1] | 533.8 | ¹H NMR (400 MHz, DMSO) δ 11.65 (s, 1H), 8.67 (d, J = 5.4 Hz, 1H), 8.53 (s, 1H), 8.48 (d, J = 2.0 Hz, 1H), 7.86 (dd, J = 8.7, 6.8 Hz, 1H), 7.81 (dd, J = 5.5, 2.1 Hz, 1H), 7.12 (dd, J = 8.7, 3.4 Hz, 1H), 3.82 (s, 1H), 3.19 (s, 3H), 2.50 (s, 3H), 2.24 (s, 3H). |
| 127C | 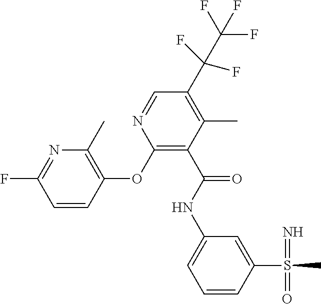 [SMILES: Cc1nc(F)ccc1Oc1ncc(C(F)(F)C(F)(F)F)c(C)c1C(=O)Nc1cccc([S@](C)(=N)=O)c1] | 532.8 | ¹H NMR (400 MHz, CDCl₃) δ 8.41 (s, 1H), 8.31 (s, 1H), 8.24 (d, J = 8.1 Hz, 1H), 8.17 (s, 1H), 7.81 (d, J = 7.9 Hz, 1H), 7.61 (dt, J = 12.2, 8.3 Hz, 2H), 6.82 (dd, J = 8.6, 3.6 Hz, 1H), 3.15 (s, 3H), 2.59 (d, J = 2.5 Hz, 3H), 2.34 (s, 3H). |
| 128C | 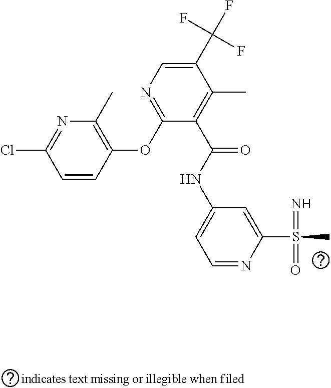 [SMILES: Cc1nc(Cl)ccc1Oc1ncc(C(F)(F)F)c(C)c1C(=O)Nc1ccnc([S@](C)(=N)=O)c1] [symbol] | 499.8 | ¹H NMR (400 MHz, DMSO) δ 11.65 (s, 1H), 8.66 (d, J = 5.4 Hz, 1H), 8.58 (s, 1H), 8.47 (d, J = 2.0 Hz, 1H), 7.81 (dd, J = 5.4, 2.1 Hz, 1H), 7.75 (d, J = 8.5 Hz, 1H), 7.46 (d, J = 8.5 Hz, 1H), 3.82 (s, 1H), 3.18 (s, 3H), 2.50 (s, 3H), 2.27 (s, 3H). |
| 129C | 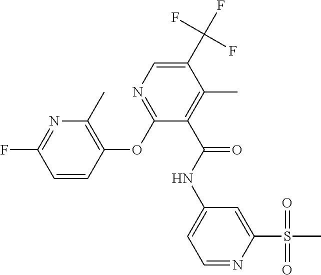 [SMILES: Cc1nc(F)ccc1Oc1ncc(C(F)(F)F)c(C)c1C(=O)Nc1ccnc(S(C)(=O)=O)c1] | 485.0 | ¹H NMR (400 MHz, CDCl₃) δ 9.03 (s, 1H), 8.69 (d, J = 5.5 Hz, 1H), 8.48 (d, J = 5.5 Hz, 1H), 8.41 (s, 1H), 7.94 (d, J = 2.0 Hz, 1H), 7.64-7.50 (m, 1H), 6.82 (dd, J = 8.6, 3.6 Hz, 1H), 3.08 (s, 3H), 2.60-2.55 (m, 3H), 2.31 (s, 3H). |

[symbol]

indicates data missing or illegible when filed

### Example 130C

### Compound Profiling Human NaV1.8/03 Cell Line—SyncroPatch384PE Assay

Compounds were tested on recombinant human Nav1.8/b3 stably transfected CHO cells using the SyncroPatch384PE system (Nanion Technologies), an automated patch clamp device. Cells were cultured at 37° C./5% CO₂ in Ham's F-12 supplemented with 1O⁰% fetal bovine serum, 100 U/mL penicillin G sodium, 100 mg/mL streptomycin sulfate and selection antibiotics (0.01 mg/ml Blasticidin, 0.4 mg/mi Zeocin and 0.25 mg/mi Hygromycin). On the day of the recordings, cells in culture dishes were washed twice with Hank's Balanced Salt Solution (HBSS) and treated with Accutase for approximately 20 minutes. Immediately before use in the SynchroPatch384PE using an 8-hole NPC-384 chip, the cells were washed in HBSS to remove the Accutase and resuspended in extracellular solution. All experiments were performed at ambient temperature. Intracellular solution contained (mM): CsCl, 50; CsF, 90; MgCl₂, 5; EGTA, 1; HEPES, 10; pH adjusted to 7.2 with CsOH. Extracellular solution contained (mM): NaCl, 137; KCl, 4.0; CaCl₂), 3.8; MgCl₂, 1; HEPES, 10; Glucose, 10; pH adjusted to 7.4 with NaOH. 100 nM tetrodotoxin (TTX) was added to the extracellular solution to block endogenous TTX-sensitive sodium currents.

Compounds were tested in quadruplicate in 0.3% DMSO and 0.03% pluronic Acid. Compounds were diluted 1:3.33 in extracellular solution to create an 8-point concentration response curve. Each plate contained a historical positive control and up to ten compounds. 300 μM tetracaine and 0.3% DMSO+0.03% pluronic acid were used as high and low controls respectively.

Whole cell patch clamp recordings were conducted according to Nanion's standard procedure for SyncroPatch384PE®. Cells were held at a holding potential of −120 mV. A depolarization step to 10 mV for 30 ms was applied (P1 measurement), followed by a hyperpolarization step to −100 mV for 100 ms. An inactivation step at −35 mV for 10 sec was applied before stepping to −100 mV for 20 ms, followed by a step to 10 mV for 30 ms (P2 measurement) and then back to −100 mV for 30 ms. Sweep interval was 15 sec. Following establishment of the whole-cell configuration in extracellular solution, cells were washed in extracellular solution containing 0.3% DMSO and 0.03% pluronic acid to stabilize the baseline current. Compounds were then applied by the SynchroPatch384 PE system into each well and the current was recorded for five minutes in extracellular solution, followed by application of tetracaine to achieve full block at the end of experiment. The potency of the compounds was assessed on two read-outs, resting state block (P1 measurement) or inactivated state block (P2 measurement) to obtain IC50 values. Values were normalized to high (tetracaine) and low (DMSO+pluronic acid) controls.

The Table below shows the potency of compounds against human Nav1.8, where “A” represents an IC50 less than or equal to 5 nM, “B” represents an IC50 greater than 5 nM to less than or equal to 50 nM, “C” represents an IC50 greater than 50 nM to less than or equal to 100 nM, “D” represents an IC50 greater than 100 nM to less than or equal to 200 nM, “E” represents an IC50 greater than 200 nM.

| Example | P1 | P2 |
|---|---|---|
| 1C | C | C |
| 2C | B | B |
| 3C | B | B |
| 4C | B | B |
| 5C | A | A |
| 6C | A | A |
| 7C | A | A |
| 8C | A | A |
| 9C | C | C |
| 10C | B | B |
| 11C | B | B |
| 12C | C | C |
| 13C | A | A |
| 14C | B | B |
| 15C | B | B |
| 16C | E | E |
| 17C | E | E |
| 20C | B | B |
| 21C | A | A |
| 22C | C | C |
| 23C | A | A |
| 24C | A | A |
| 25C | A | A |
| 26C | B | B |
| 27C | C | C |
| 28C | A | A |
| 29C | E | E |
| 30C | E | E |
| 31C | D | D |
| 32C | C | C |
| 33C | B | A |
| 34C | E | E |
| 35C | A | A |
| 36C | E | E |
| 37C | E | E |
| 38C | E | D |
| 39C | E | E |
| 40C | A | A |
| 43C | A | A |
| 44C | B | B |
| 45C | A | A |
| 48C | E | E |
| 86C | B | B |
| 87C | B | B |
| 88C | A | A |
| 90C | A | A |
| 91C | A | A |
| 92C | A | A |
| 93C | D | D |
| 94C | B | B |
| 95C | A | A |
| 96C | A | A |
| 97C | C | C |
| 98C | B | B |
| 99C | B | B |
| 100C | B | B |
| 101C | B | B |
| 102C | B | B |
| 103C | C | C |
| 104C | C | C |
| 105C | A | A |
| 106C | A | A |
| 107C | A | A |
| 108C | A | A |
| 109C | A | A |
| 110C | B | B |
| 111C | E | E |
| 112C | E | E |
| 113C | B | B |
| 114C | E | E |
| 115C | E | E |
| 116C | B | B |
| 117C | E | E |
| 118C | A | A |
| 119C | A | A |
| 120C | B | B |
| 121C | C | C |
| 122C | B | B |
| 123C | A | A |
| 124C | B | B |
| 125C | E | E |

### Example 131C

### Compound Profiling Human NaV1.8/β1 Cell Line—Sophion QPatch II Assay

Compounds were tested on recombinant human Nav1.8/β1 stably transfected HEK293 (Eurofins, CYL3025, St. Charles, MO) cells using the QPatch II system (Sophion Bioscience A/S, Ballerup—Denmark), an automated patch clamp device. Cells were cultured at 37° C./5% CO₂ in DMEM/F-12 supplemented with 10% fetal bovine serum, 1× Non-Essential Amino Acids, and selection antibiotics (0.625 μg/mL Puromycin, 400 μg/mL Geneticin, and 100 μg/mL Hygromycin). One or two days prior to recording, cells were moved to a lower temperature 30° C./5% CO₂ incubator to increase surface expression of the channels. On the day of the recordings, cells in culture dishes were washed twice with Ca²⁺—Mg²⁺ free DPBS and treated with Detachin (Genlantis, San Diego) for 3 minutes and then resuspended in Serum Free Medium (EX-CELL® ACF CHO Medium for Cell Culture (Sigma-Aldrich) supplemented with 0.04 mg/mL soybean trypsin Inhibitor and 25 mM HEPES (all components supplied by Sigma-Aldrich, St. Louis, MO) and allowed to recover for a minimum of 20 minutes in the onboard QStirrer. All experiments were performed at ambient temperature. Intracellular solution contained (mM): CsCl, 50; CsF, 90; MgCl₂, 2; EGTA, 5; HEPES, 10; pH adjusted to 7.2 with CsOH. Extracellular solution contained (mM): NaCl, 137; KCl, 4; CaCl₂), 3.8; MgCl₂, 1; HEPES, 10; Glucose, 10; pH adjusted to 7.4 with NaOH. 500 nM tetrodotoxin (TTX) was added to the extracellular solution to block endogenous TTX-sensitive sodium currents.

Compounds were tested in an interleaved manner across two cohorts of wells to develop an aggregate 8-point concentration response curve. Final concentrations include 0.1% DMSO and 0.03% pluronic Acid. In general, the concentration range spans a range from 0.01-300 nM. Compounds were diluted 1:10 with extracellular solution within their respective cohort. DMSO (0.1% by volume) served as a vehicle control to ascertain assay performance.

Whole cell patch clamp recordings were conducted according to Sophion standard procedure for QPatch II®. Cells were held at a holding potential of −120 mV. A depolarizing step to 10 mV for 30 ms was applied to elicit inward Na⁺ currents. Inter-sweep interval was 15 sec. Following establishment of the whole-cell configuration in extracellular solution, cells were washed twice in extracellular solution containing 0.1% DMSO and 0.03% pluronic acid to stabilize the baseline current. Compounds were then applied by the QPatch II system into each well in an ascending manner within cohort and the current was recorded for five minutes for each concentration. Percent of current remaining was determined by taking the average of the last 5 sweeps within each concentration and normalizing to the latest pre-compound saline period. The potency (IC₅₀) values of the compounds were only assessed against resting state block. Wells whose initial sodium current magnitude were below 2 nA or whole cell resistance fell below 100 MO were excluded from analysis.

The Table below shows the potency of compounds against human Nav1.8, where “A” represents an IC50 less than or equal to 5 nM, “B” represents an IC50 greater than 5 nM to less than or equal to 50 nM, “C” represents an IC50 greater than 50 nM to less than or equal to 100 nM, “D” represents an IC50 greater than 100 nM to less than or equal to 200 nM, “E” represents an IC50 greater than 200 nM.

| Example | P1 |
|---|---|
| 41C | B |
| 42C | E |
| 46C | E |
| 47C | E |
| 49C | E |
| 50C | B |
| 51C | B |
| 52C | B |
| 53C | B |
| 54C | C |
| 55C | B |
| 56C | B |
| 57C | B |
| 58C | B |
| 59C | B |
| 60C | A |
| 61C | D |
| 62C | E |
| 63C | E |
| 64C | B |
| 65C | B |
| 66C | C |
| 67C | B |
| 68C | D |
| 69C | D |
| 70C | D |
| 71C | B |
| 72C | B |
| 73C | B |
| 74C | B |
| 75C | B |
| 76C | B |
| 77C | E |
| 78C | B |
| 79C | B |
| 80C | D |
| 81C | E |
| 82C | C |
| 83C | E |
| 84C | E |
| 85C | E |
| 86C | B |
| 87C | B |
| 88C | B |
| 89C | E |

### (D) Fourth Set of Compounds

In one aspect, the invention provides a compound of Formula (I):

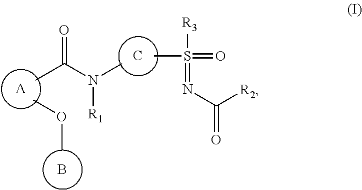

and pharmaceutically acceptable salts, hydrates and solvates thereof, wherein:
A is a substituted or unsubstituted heteroaryl ring comprising at least one heteroatom selected from a group consisting of O, S, or N;
wherein the one or more substitutions on A are selected from H, —OH, halo, C₁-C₈-alkyl, C₁-C₈ fully or partially fluorinated fluoroalkyl, C₂-C₈ branched alkyl, alkenyl, alkynyl, haloalkyl, alkoxy, cycloalkoxy, haloalkoxy, nitro, cyano, —C(R′)(R′)-cycloalkyl, C(R′)(R′)-aryl, —NR′R″, substituted or unsubstituted 3-8 membered cycloalkyl, 3-8 membered cycloalkenyl, or 3-6 membered heterocycloalkyl, wherein the 3-6 membered heterocycloalkyl comprises at least one heteroatom independently selected from O, S, and N;
B is substituted or unsubstituted aryl or heteroaryl, wherein the substitutions are selected from the group consisting of substituted or unsubstituted C₁-C₈ alkyl, deuterated C₁-C₄ alkyl wherein the alkyl chain may be fully or partially deuterated, halo C₁-C₄ alkyl where the alkyl chain is fully or partially halogenated, C₃-C₁₀ cycloalkyl, halogen, cyano, nitro, C₁-C₈ alkoxyl, haloalkoxyl wherein the haloalkyoxyl chain may be fully or partially halogenated, and arylalkoxyl;
C is substituted or unsubstituted aryl or heteroaryl, wherein the one or more substitutions are independently selected from a group consisting of halo, C₁-C₈ alkyl, haloalkyl, alkoxy;
R₁ is selected from a group consisting of: H and C₁-C₄ alkyl;
R₂ is selected from a group consisting of: —(CH₂)ₙRₐ, —(CR_{b}R_{c})ₙRₐ, —(CR_{b}R_{c})ₙ—(OCH₂CH₂)ₙRₐ, —(CR_{b}R_{c})ₙ—(NR′CH₂CH₂)ₙRₐ, —(CR_{b}R_{c})ₙ—(NR′CH₂CH₂O)ₙRₐ, and —(CR_{b}R_{c})ₙ—(NHCH₂CH₂NH)ₙRₐ;
   wherein Rₐ is H, C₁-C₈-alkyl or branched alkyl, C₃-C₈ cycloalkyl, aryl, heteroaryl, 4-7 membered heterocyclyl, alkenyl, alkynyl, haloalkyl, OH, alkoxy, cycloalkoxy, haloalkoxy, —(OCH₂CH₂)ₙR_{b}, —NR′R″, —COONR′R″, —COOR′R″, alkylsulfonyl, arylsulfonyl, or —SO₂NR′R″;
   R_{b} is H, F, C₁-C₆-alkyl, haloalkyl, branched alkyl, aryl, heteroaryl, 3-7 membered carbocyclyl or heterocyclyl with one or more heteroatoms;
   R_{c} is H, C₁-C₆ alkyl, or F;
   R_{c} and R_{b} together optionally form a 3-6 member carbocyclic or heterocyclic ring;
R₃ is selected from a group consisting of C₁-C₃ alkyl, C₃-C₄ cycloalkyl, haloalkyl, halocycloalkyl, aryl, heteroaryl, and heterocyclyl.

In another embodiment, the compound is a compound of Formula (II):

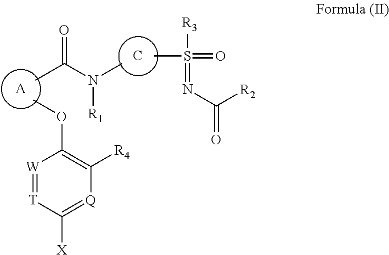

wherein: A, C, R₁, R₂, and R₃ are described above, Q, T and W are independently N or CR₅;
R₅ is H, halogen, —CD₃, alkyl, cycloalkyl, haloalkyl, alkoxy, haloalkoxy, cyano, —CF₃, —OCF₃, or cycloalkoxy;
R₄ is H, —OH, halo, —CD₃, C₁-C₆-alkyl, branched alkyl, haloalkyl where the alkyl chain is fully or partially halogenated, alkoxy, arylalkoxy, cycloalkoxy, haloalkoxy, cyano, —CH₂-cycloalkyl, —CH(CH₃)-cycloalkyl, trifluoromethyl, cyclopropylmethyl, 3-6 membered cycloalkyl or 3-6 membered heterocycloalkyl, any of which may have one or more substituents, wherein the 3-6 membered heterocycloalkyl comprises at least one heteroatom independently selected from O, S, and N;
X is H, halo, —CD₃, alkyl, haloalkyl, alkoxy, cycloalkoxy, haloalkoxy, cyano, —CF₃, or —OCF₃.

In another embodiment, the compound is a compound of Formula (III):

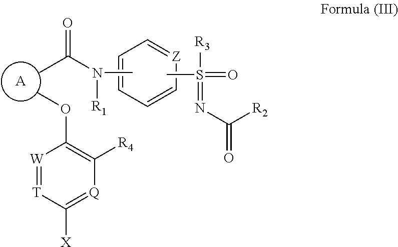

wherein, Z is CH, N, CF, or NO; and A, W, T, Q, X, R₁, R₂, R₃, and R₄ are described above.

In other embodiments, in the compounds of Formula (I), Formula (II), and/or Formula (III), ring A is substituted or unsubstituted 5-6 membered heteroaryl with one or more heteroatom. The 5-6 membered ring heteroaryl includes N as the one or more heteroatom. The heteroatom N may be in the form of an N-oxide, wherein the N-oxide is selected from a group consisting of: pyridyl N-oxide, pyrazinyl N-oxide, pyridazinyl N-oxide, and pyrimidinyl N-oxide.

In other embodiments, A is a 6-membered heteroaryl comprising at least one heteroatom, wherein the heteroatom is N. In certain embodiments, A is a 6-membered heteroaryl comprising 2 N atoms.

In other embodiments, in the compounds of Formula (I), Formula (II), and/or Formula (III), ring A may be a substituted or unsubstituted pyridyl, pyrimidinyl, pyrazinyl, or pyridazinyl.

In other embodiments, in the compounds of Formula (I), Formula (II), and/or Formula (III), ring A is:

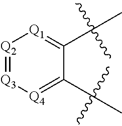

wherein Q₁, Q₂, Q₃ and Q₄ are independently selected from a group consisting of: N, N⁺O⁻, or CR₆; wherein at least two of Q₁, Q₂, Q₃ and Q₄ are CR₆; R₆ is H, —OH, halo, —CD₃, alkyl, haloalkyl, alkoxy, haloalkoxy, cyano, —CF₃, —OCF₃, substituted or unsubstituted 5 or 6 membered ring heterocyclyl or heteroaryl, saturated heterocyclyl, or partially unsaturated heterocyclyl, O-aryl, O-heteroaryl, O-cycloalkyl, or O-cycloheteroalkyl.

In other embodiments, in the compounds of Formula (I), Formula (II), and/or Formula (III), ring A is:

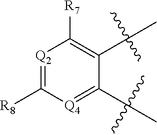

wherein,
Q₂ and Q₄ are independently N or N⁺O⁻;
Q₂ is N or N⁺O⁻ and Q₄ is CR₆; or
Q₂ is CR₆ and Q₄ is N or N⁺O⁻;
R₇ and R₈ are independently selected from the group consisting of H, —OH, halo, —CD₃, substituted or unsubstituted C₁-C₆ alkyl, branched alkyl, alkenyl, alkylnyl, haloalkyl, alkoxy, cycloalkyl, heterocyclyl, NH₂, NHR′, NR′R″, aryl, Heteroaryl, —CF₂CH₃, and —CF₂CF₃;
R₆ is described above.

In other embodiments, in the compounds of Formula (I), Formula (II), and/or Formula (III), ring A is:

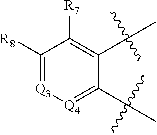

wherein,
Q₃ and Q₄ are N;
Q₃ is N, N⁺O⁻; Q₄ is CR₆; or
Q₃ is CR₆, Q₄ is N or N⁺O⁻; wherein R₇ and R₈ are defined above.

In other embodiments, in the compounds of Formula (I), Formula (II), and/or Formula (III), ring A is:

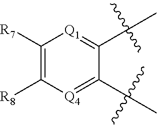

wherein
Q₁ and Q₄ are independently N or N⁺O⁻;
Q₁ is N or N⁺O⁻ and Q₄ is CR₆; or
Q₁ is CR₆ and Q₄ is N or N⁺O⁻; wherein R₆, R₇, and R₈ are described above.

In other embodiments, in the compounds of Formula (I), Formula (II), and/or Formula (III), ring A is:

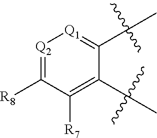

wherein Q₁ and Q₂ is N; wherein R₇ and R₈ are described above.

In other embodiments, in the compounds of Formula (I), Formula (II), and/or Formula (III), ring A is:

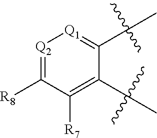

wherein Q₁ is CR₆, and Q₂ is N; R₆, R₇, and R₈ are described above.

In other embodiments, in the compounds of Formula (I), Formula (II), and/or Formula (III), ring A is:

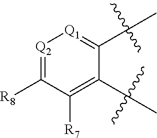

wherein, Q₁ is N; Q₂ is CR₆; R₆, R₇, and R₈ are described above.

In other embodiments, in the compounds of Formula (I), Formula (II), and/or Formula (III), R₁ is H.

In other embodiments, in the compounds of Formula (I), Formula (II), and/or Formula (III), R₃ is methyl.

In other embodiments, in the compounds of Formula (I), Formula (II), and/or Formula (III), R₂ is H, methyl, —CH₂—NH₂, —CH₂—NH—CH₃, —CH₂—OH, —CH(NH₂)(CH₃), —CH₂—OH, —CH(OH)(CH₃), —CH(CF₃)(NH₂), —CH₂—O—CH₃, amino-cycopropyl, pyrrolidine, azetidine, oxetane, tetrahydrofuran, or hydroxypyrrolidone.

In other embodiments, in the compounds of Formula (II) and/or Formula (III), T is N. In other embodiments, T is N, W is CH, and Q is CH.

In other embodiments, in the compounds of Formula (II) and/or Formula (III), R₄ is methyl. In other embodiments, in the compounds of Formula (II) and/or Formula (III), X is F.

In other embodiments, in the compounds of Formula (II) and/or Formula (III), Z is N or CH.

In other embodiments, the compound of the invention is selected from a group consisting of:

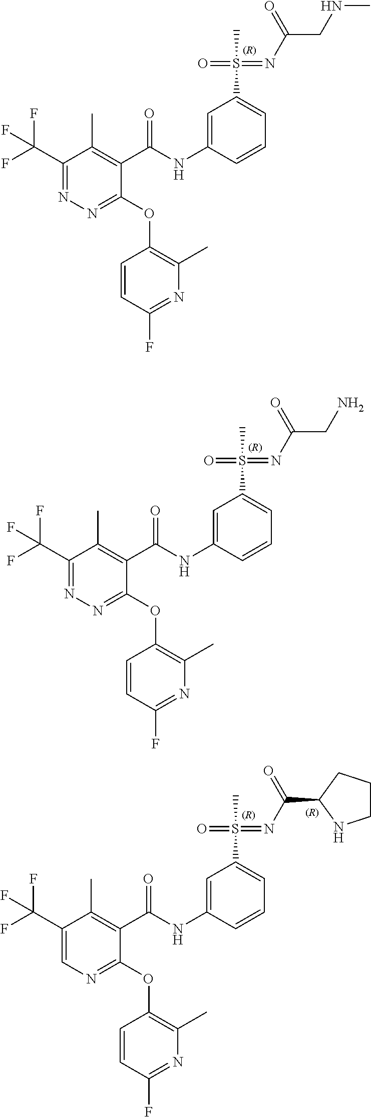

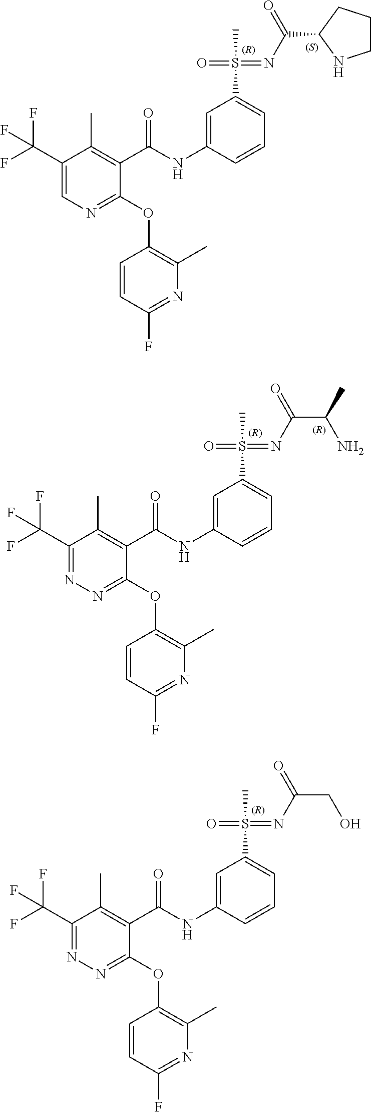

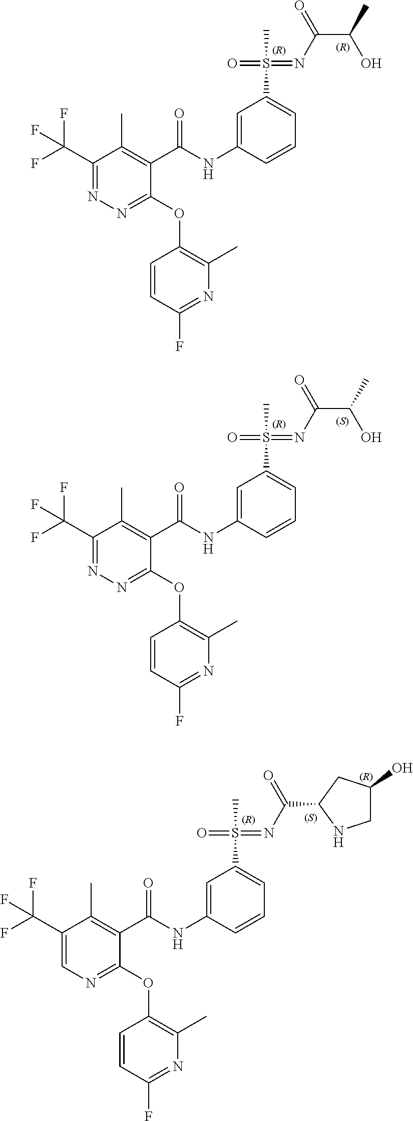

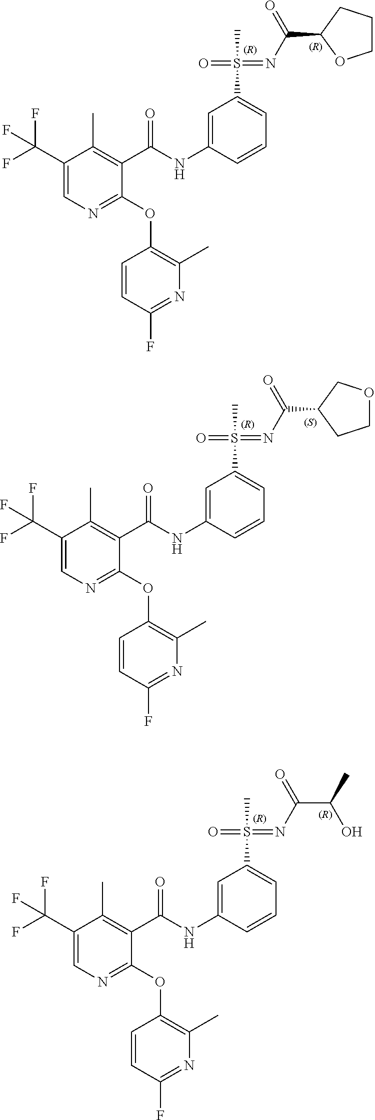

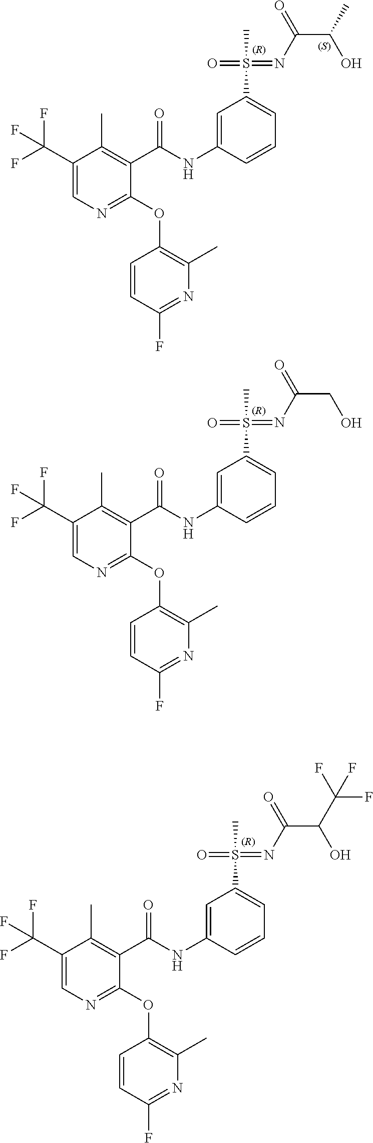

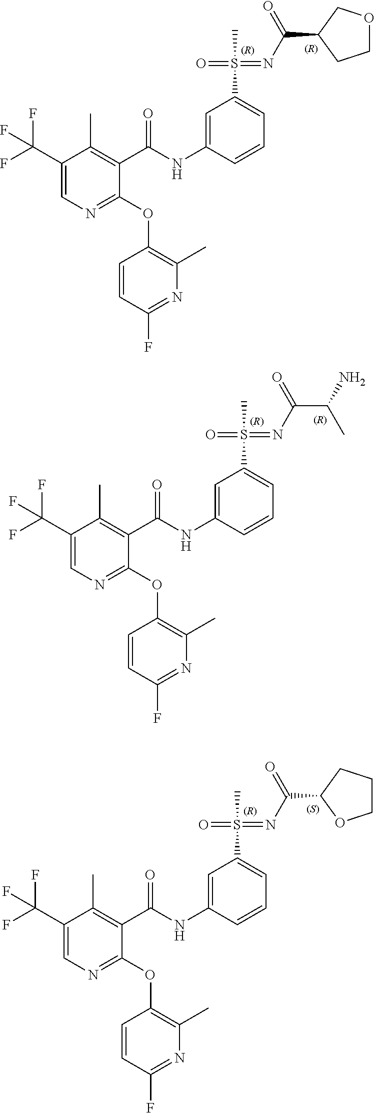

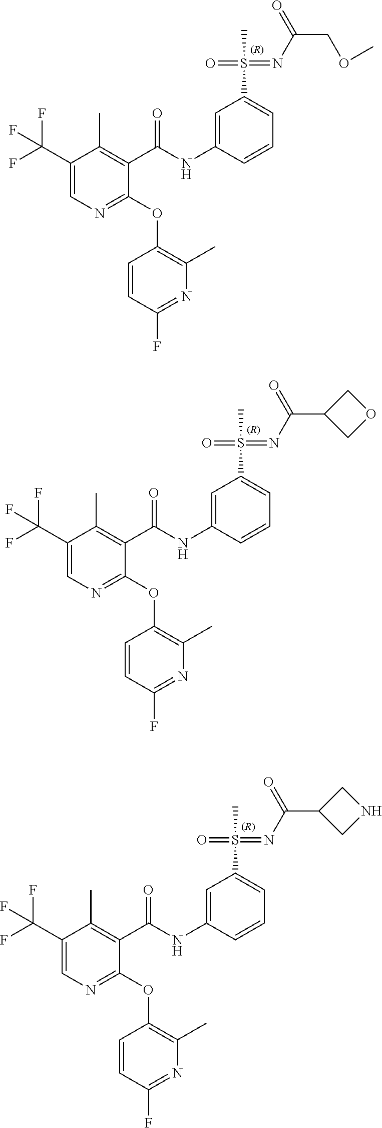

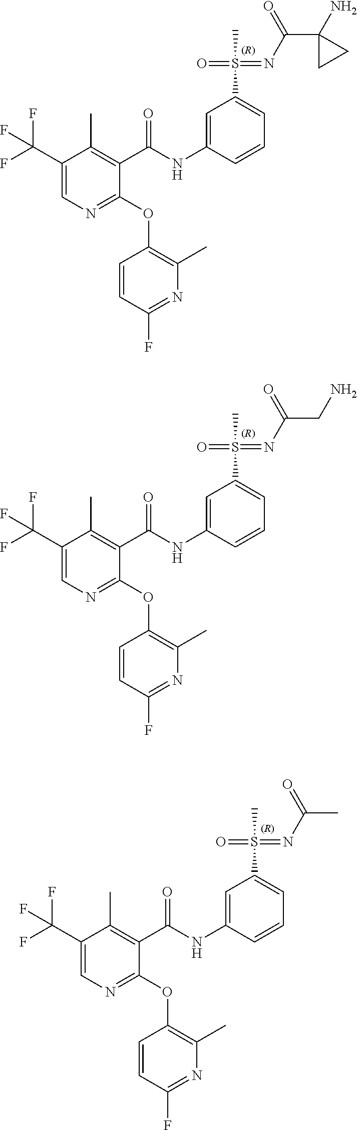

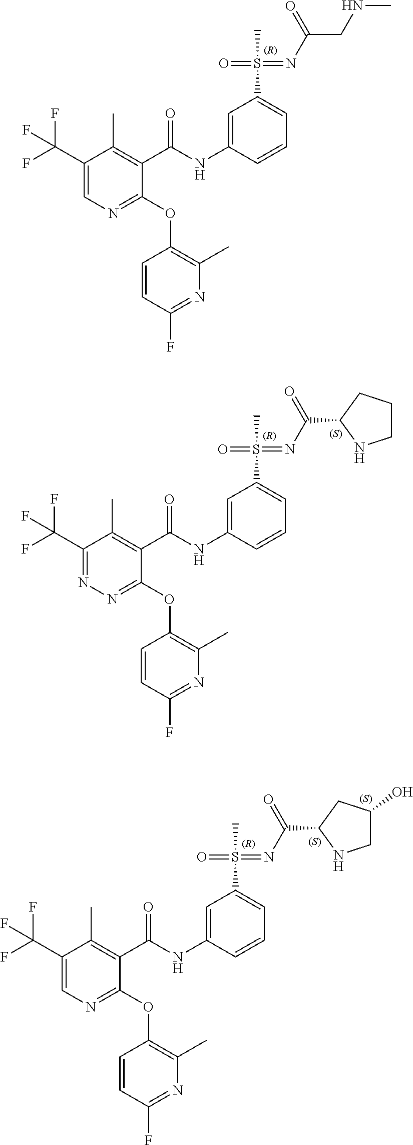

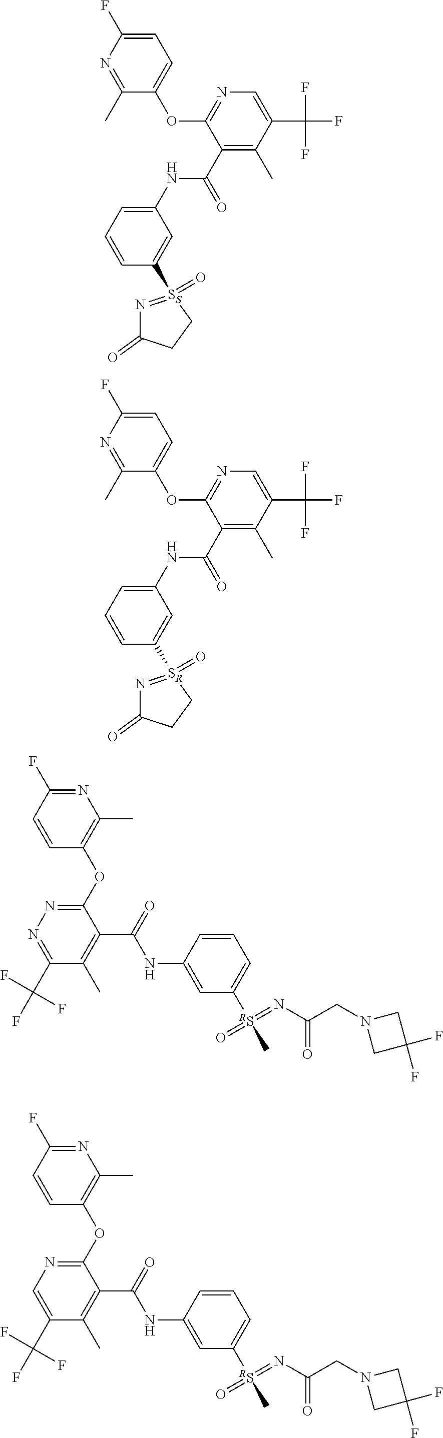

The fourth set of compounds, provided herein, are prepared by methods and procedures described below. The intermediates described in this section may be relied upon for preparation of fourth set of compounds.

Chemicals were purchased from standard commercial vendors and used as received unless otherwise noted. Otherwise, their preparation is facile and known to one of ordinary skill in the art, or it is referenced or described herein. Abbreviations are consistent with those in the ACS Style Guide. “dry” glassware means oven/desiccator dried. Solvents were ACS grade unless otherwise noted.

All reactions were performed in flame-dried or oven-dried glassware under a positive pressure of dry nitrogen or dry argon and were stirred magnetically unless otherwise indicated. Chemicals were purchased from standard commercial vendors and used as received unless otherwise noted. Yields are not optimized. The chemical names were generated using the ChemDraw Professional 19.1, available from PerkinElmer or chemAxon.

Reactions were monitored by thin layer chromatography (TLC) using 0.25 mm silica gel 60 F254 plates purchased from EMD MILLIPORE™. Purification was performed with Biotage Isolera One Flash Chromatography Instrument or purified using one of the preparative HPLC methods mentioned below.

### Prep Method 1

Equipment: Shimadzu LCMS 2020 mass-directed preparative HPLC System; column: Gemini 5 um C₁₈ column, 150*21.2 mm; General gradient: 30% to 90% MeCN/H₂O containing 0.1% HCOOH, gradient may be slight adjusted for specific compound; Flow rate: 20 mL/min; Column temperature: ambient temperature; UV Wavelength: 214 and 254 nm;

### Prep Method 2

Equipment: Shimadzu LC-20AP Preparative HPLC System; column: Gemini 5 um C₁₈ column, 150*21.2 mm; General gradient: 30% to 90% MeCN/H₂O containing 0.1% TFA, gradient may be slight adjusted for specific compound; Flow rate: 20 mL/min; Column temperature: ambient temperature; UV Wavelength: 214 and 254 nm.

### Prep Method 3

Equipment: Shimadzu LC-20AP Preparative HPLC System; column: Gemini 5 um C₁₈ column, 150×21.2 mm; General gradient: 30% to 90% MeCN/H₂O containing 0.05% ammonia, gradient may be slight adjusted for specific compound; Flow rate: 20 mL/min; Column temperature: ambient temperature; UV Wavelength: 214 and 254 nm.

Analytical LCMC were collected using one of following methods—

### Method 1

Equipment: Shimadzu LCMS 2020 Mass Spectrometer; Column: HALO C₁₈ 2.7 μm, 3.0 mm×30 mm; Mobile Phase: MeCN (0.05% HCOOH)—Water (0.05% HCOOH); Gradient: MeCN from 5% to 95% over 1.4 min, hold 0.6 min, total run time is 2.5 min; Flow rate: 1.8 mL/min; Column temperature: 50° C.; Wavelength: 214 and 254 nm PDA.

### Method 2

Equipment: Shimadzu LCMS 2020 Mass Spectrometer; XBridge BEH C₁₈ 2.5 μm, 3.0 mm×30 mm Mobile Phase: MeCN—Water (0.1% NH₄OH); Gradient: MeCN from 5% to 95% over 1.8 min, hold 0.7 min, total run time is 3.0 min; Flow rate: 1.0 mL/min; Column temperature: 50° C.; Wavelength: 214 and 254 nm PDA.

### Method 3

Equipment: Shimadzu LCMS 2020 Mass Spectrometer; Column: HALO C₁₈ 2.7 μm, 3.0 mm×30 mm Mobile Phase: MeCN (0.05% TFA)—Water (0.05% TFA); Gradient: MeCN from 5% to 95% over 1.4 min, hold 0.6 min, total run time is 2.5 min; Flow rate: 1.8 mL/min; Column temperature: 50° C.; Wavelength: 214 and 254 nm PDA.

SFC chiral resolution was performed on Shimadzu Nexera UC Preparative SFC System (SFE-30A, LC-30AD_{SF}, SFC-30A) using following methods:

### Method 1

Column: Daicel chiralpak-AS-H 5 μm 250×20 mm; Mobile Phase: CO₂/MeOH [0.1% NH₃ (7M in MeOH)], CO₂/MeOH ratio varies for different compounds; Oven temperature: 40° C.; Flow rate: 38 mL/min.

### Method 2

Column: Daicel chiralpak-OJ-H 5 μm 250*20 mm; Mobile Phase: CO₂/MeOH (0.1% HCOOH), CO₂/MeOH ratio varies for different compounds; Oven temperature: 40° C.; Flow rate: 38 mL/min.

### Method 3

Column: Daicel chiralpak-OD-H 5 μm 250×20 mm; Mobile Phase: CO₂/MeOH, ratio varies for different compounds; Oven temperature: 40° C.; Flow rate: 38 mL/min.

### Method 4

Column: Daicel chiralpak-AD-H 5 μm 250×20 mm; Mobile Phase: CO₂/i-PrOH, ratio varies for different compounds; Oven temperature: 40° C.; Flow rate: 38 mL/min.

### Method 5

Column: Daicel chiralpak-IC 5 μm 250×20 mm; Mobile Phase: CO₂/EtOH, ratio varies for different compounds; Oven temperature: 40° C.; Flow rate: 38 mL/min.

Unless otherwise stated, ¹H nuclear magnetic resonance spectroscopy (NMR) spectra were recorded on a Bruker AVANCE NEO 400 MHz Digital NMR Spectrometer. Chemical shifts, 6, are quoted in parts per million (ppm) relative to TMS and calibrated using residual un-deuterated solvent as an internal reference. The following abbreviations are used to denote the multiplicities and general assignments: s (singlet), d (doublet), t (triplet), q (quartet), dd (doublet of doublets), ddd (doublet of doublet of doublets), dt (doublet of triplets), dq (doublet of quartets), hep (heptet), m (multiplet), pent (pentet), td (triplet of doublets), qd (quartet of doublets), app. (apparent) and br. (broad). Coupling constants, J, are quoted to the nearest 0.1 Hz.

### General Synthetic Schemes

Several methods for preparing the compounds of this invention are illustrated in the following schemes and examples. The present invention further provides processes for the preparation of compounds of structural Formula I as defined above. In some cases, the order of carrying out the foregoing reaction schemes may be varied to facilitate the reaction or to avoid unwanted reaction products. The following examples are provided for the purpose of illustration only and are not to be construed as limitations on the disclosed invention.

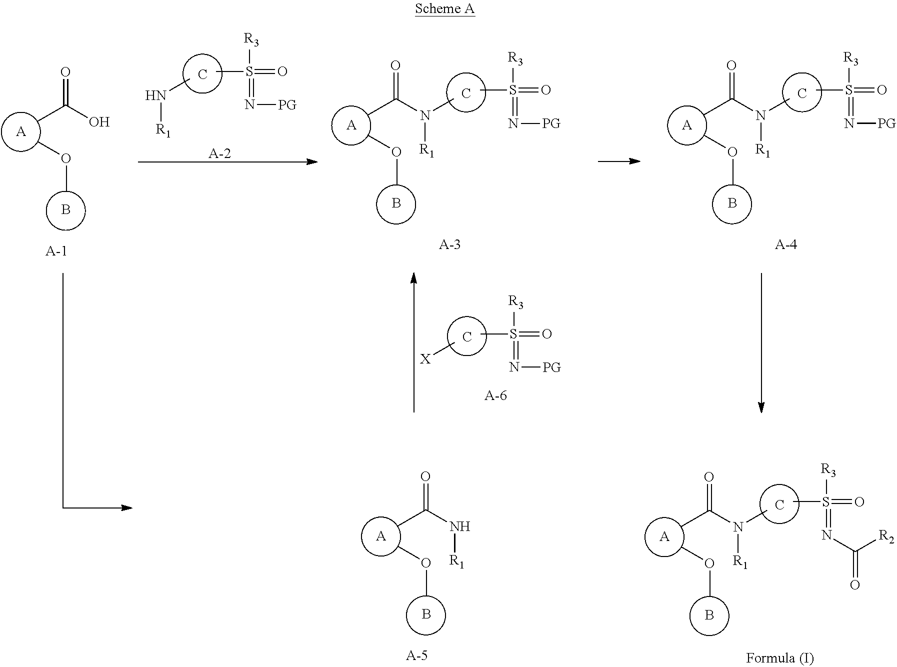

As illustrated in Scheme A, in general, compounds of Formula (I) may be synthesized starting from carboxylic acids A-1 by reacting with a substituted aniline or heteroaryl aniline A-2 using standard amide coupling reagents, not limited to HATU, TBTU, EDC or T3P in organic solvents and base, such as DIEA, to give intermediates of type A-3. Intermediates A-3 may contain a protecting group (PG) such as Boc which may be removed by treatment with an acid such as TFA to provide intermediates having structure A-4. Any suitable PG such as Cbz or Fmoc may be employed and can be removed accordingly. The NH group of A-4 may be acylated using standard amide coupling reagents such as HATU in conjunction with a carboxylic acid and a base such as DIEA or the NH group may be acylated with a reactive carboxylic acid ester such as the N-hydroxysuccinimide ester, to provide compounds of Formula (I) after removal of any protecting groups. There are numerous methods to acylate the NH group which can be effected by those skilled in the art. If desired, Compounds of Formula (I) may be further modified to provide additional Compounds of Formula (I). Alternatively, carboxylic acid A-1 may be treated with ammonia or a primary amine (R₁NH₂) in the presence of an amide coupling reagent such as HATU and a base such as DIEA to give carboxamide intermediate A-5 which can undergo metal-catalyzed coupling with a halogen-substituted aryl or heteroaryl compounds of type A-6 to give compounds of formula A-3. Compounds of Formula (I) can then be obtained from A-3 as described.

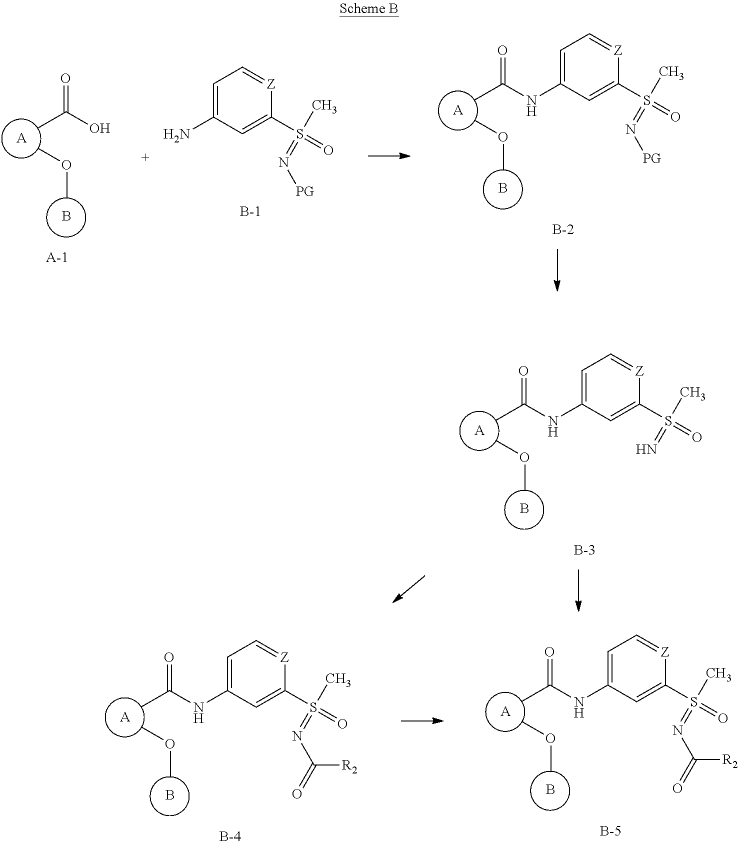

As further illustrated in Scheme B, in general, compounds of the invention can be prepared by reacting intermediates A-1 with an amine B-1, utilizing amide coupling conditions or by activation of appropriately functionalized carboxylic acid A-1 with (COCl)₂ or POCl₃ and with amine B-1 and base such as DIEA or pyridine in DCM, DMF or THF to give compounds of type B-2. The compounds of formula B-3 can be formed by removing a protecting group, such as Boc under acidic conditions. In some instances, B-3 can be separated to the corresponding R and S isomers using chiral HPLC. R- and S-isomers also can be prepared by coupling of the acid to enantiomerically pure amine B-1 followed deprotection. The sulfoximine nitrogen of intermediate B-3 was acylated using standard amide coupling reagents such as, not limited to, HATU, a base such as DIEA, and HO₂C—R₂-PG to give compounds of formula B-4. When B-4 contains a PG, as shown, it was removed to give a compound of formula B-5. For example, if a Boc group as used to protect the sulfoximine, it was removed by treatment with TFA to give compounds of formula B-5. Trialkylsilyl groups may be employed to protect hydroxyl groups and can be removed by treatment with aqueous acids such as acetic acid. B-3 may yield compound B-5 directly if there is no need for a protecting group.

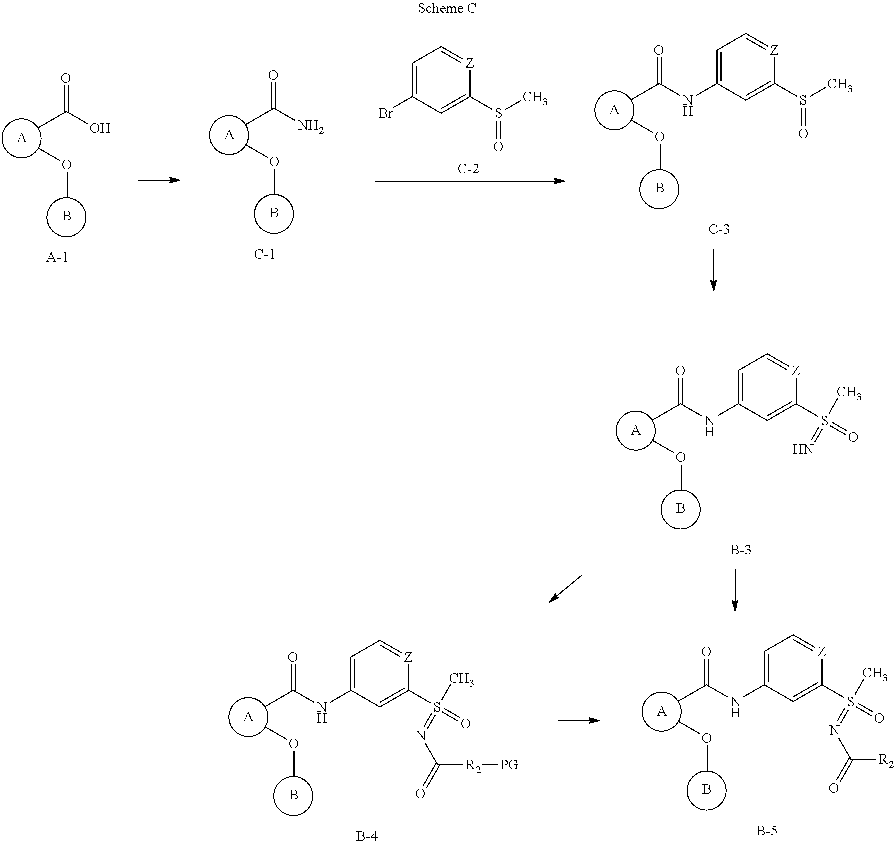

As illustrated in Scheme C, in general, compounds of the invention can be prepared by activation of appropriately functionalized carboxylic acid A-1 in organic solvent with either (COCl)₂ or SOCl₂ followed by addition of NH₄OH to afford C-1. Intermediate C-1 can then be brought together with materials of variously substituted Br compounds C-2, utilizing Xantphos-Pd-G2 mediated coupling conditions to deliver intermediate C-3. The compounds of formula C-3 may be treated with ammonium carbonate or ammonium carbamate and (diacetoxyiodo)benzene (PIDA) in methanol to provide compounds of formula B-3. In some instances, B-3 was separated to the corresponding R and S isomers using chiral HPLC conditions. Compounds of formula B-4 were obtained using standard amide coupling reagents such as, not limited to, HATU, a base such as DIEA, and H₀₂C—R₂-PG. When the protecting group PG was Boc, it was removed by treatment with TFA to give compounds of formula B-5. Trialkylsilyl groups may be employed to protect hydroxyl groups and can be removed by treatment with aqueous acids such as acetic acid. B-3 may yield compound B-5 directly if there is no need for a protecting group.

The above schemes are intended to be illustrative and not limiting in any way. Those skilled in the art are able to prepare the Compounds of Formula (I) using these general schemes as a guideline but other methods are available to accomplish the synthesis of compounds of the invention. Specific methods are provided for each of the Examples to further illustrate the synthesis of Compounds of Formula (I).

### Specific Synthesis

### Intermediate 1

### Methyl2-chloro-4-methyl-5-nitronicotinate

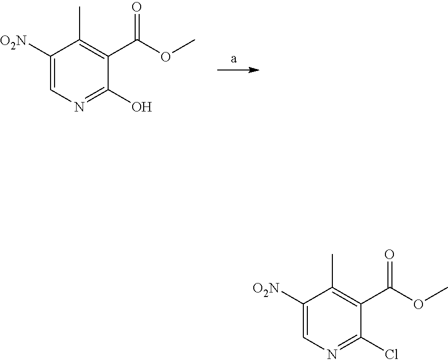

Reagents & conditions: a) phenyl dichlorophosphate, 160° C.

A solution of methyl 2-hydroxy-4-methyl-5-nitronicotinate (4.0 g, 18.9 mmol) in phenyl dichlorophosphate (20 mL) was heated to 160° C. for 3 h. The resulting solution was cooled to room temperature, quenched with water (100 mL) and extracted with EtOAc (50 mL×3). The combined organic phases were washed with brine, dried over sodium sulfate, concentrated under vacuum. The residue was purified by flash column chromatography on silica gel (PE/EtOAc=10/1) to provide methyl 2-chloro-4-methyl-5-nitronicotinate (3 g, 69%) as a light-yellow solid. LCMS (ESI) calcd. for C₈H₈ClN₂O₄ [M+H]⁺ m/z 231.02, found 230.85.

### Intermediate 2

### (S)-2-((tert-butyldimethylsilyl)oxy)propanoic acid

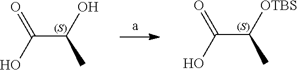

Reagents & conditions: TBSCl, imidazole, DCM

To a solution of (S)-2-hydroxypropanoic acid (1 g, 11.11 mmol) and imidazole (2.27 g, 33.32 mmol) in DCM (15 mL) was added TBSCl (2.00 g, 13.33 mmol) at 0° C. Then the mixture was heated at 25° C. for 16 hours. After the rection was completed, the mixture was diluted with water and extracted with DCM (3×30 mL). The combined organic layers were washed with brine, dried with Na₂SO₄, and concentrated under vacuum to give crude (S)-2-((tert-butyldimethylsilyl)oxy)propanoic acid (1.4 g) which was used directly in next step without further purification.

### Intermediate 3

### (R)-2-((tert-butyldimethylsilyl)oxy)propanoic acid

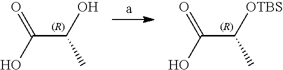

Reagents & conditions: TBSCl, imidazole, DCM

To a solution of (R)-2-hydroxypropanoic acid (1 g, 11.11 mmol) and imidazole (2.27 g, 33.32 mmol) in DCM (15 mL) was added TBSCl (2.00 g, 13.33 mmol) at 0° C. Then the mixture was warmed to RT and stirred for 16 hours. After the rection was completed, the mixture was diluted with water and extracted with DCM (3×30 mL). The combined organic layers were washed with brine, dried with Na₂SO₄, and concentrated under vacuum to give crude (R)-2-((tert-butyldimethylsilyl)oxy)propanoic acid (1.6 g) which was used directly in next step without further purification.

### Intermediate 4

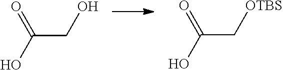 [SMILES: O=C(O)CO ; CC(C)(C)[Si](C)(C)OCC(=O)O]

2-((tert-butyldimethylsilyl)oxy)acetic acid: To a solution of 2-hydroxyacetic acid (1 g, 13.16 mmol) and imidazole (2.68 g, 39.47 mmol) in DCM (15 mL) was added TBSCl (2.38 g, 15.79 mmol) at 0° C. Then the mixture was stirred at 25° C. for 16 hours. After the rection was completed. The mixture was diluted with water and extracted with DCM (3×30 mL). The combined organic layers were washed with brine, dried with Na₂SO₄, and concentrated under vacuum to give crude 2-((tert-butyldimethylsilyl)oxy)acetic acid (1.6 g) which was used directly in next step without further purification.

### Intermediate 5

### (2S,4S)-1-(tert-butoxycarbonyl)-4-((tert-butyldimethylsilyl)oxy)pyrrolidine-2-carboxylic acid

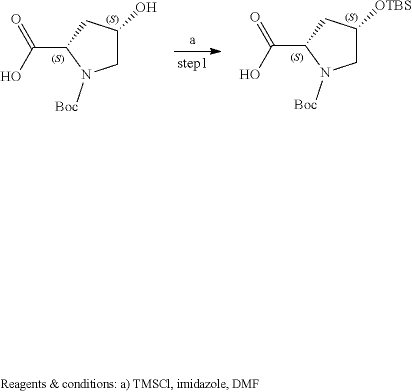

To a mixture of (2S,4S)-1-(tert-butoxycarbonyl)-4-hydroxypyrrolidine-2-carboxylic acid (1 g, 4.33 mmol) in DMF (10 mL) was added TBSCl (650 mg, 4.33 mmol) and imidazole (589 mg, 8.66 mmol). The mixture was stirred at room temperature for 16 hours. After the reaction was completed, the resulting solution was diluted with water (50 mL) and extracted with EtOAc (20 mL×3). The combined organic phases were washed with brine, dried over Na₂SO₄, concentrated under vacuum to give crude (2S,4S)-1-(tert-butoxycarbonyl)-4-((tert-butyldimethylsilyl)oxy) pyrrolidine-2-carboxylic acid (0.9 g) as a yellow oil which was used directly in next step.

### Example 1D

### (R)-2-((6-fluoro-2-methylpyridin-3-yl)oxy)-4-methyl-N-(3-(S-methyl-N-(methylglycyl)sulfonimidoyl)phenyl)-5-(trifluoromethyl)nicotinamide

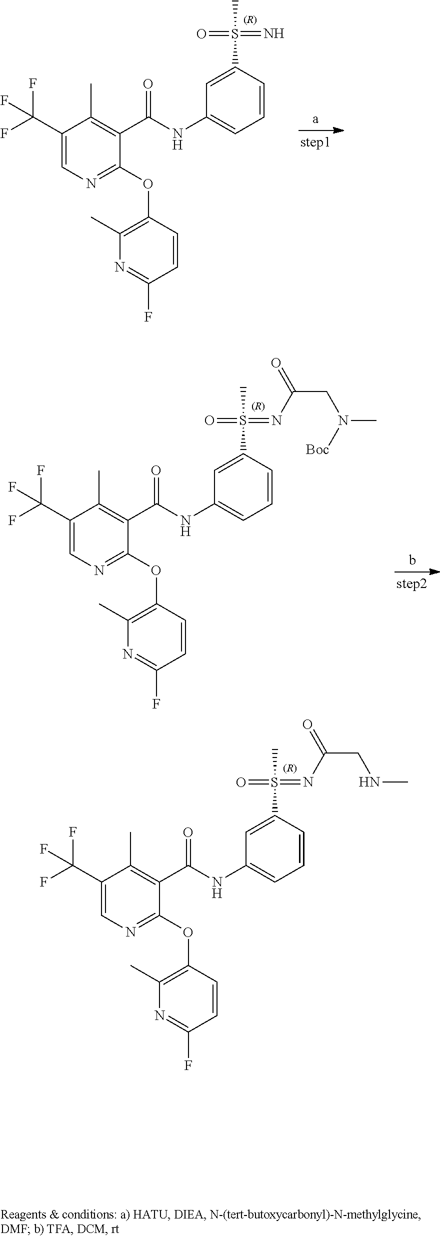

Step 1: tert-butyl (R)-(2-(((3-(2-((6-fluoro-2-methylpyridin-3-yl)oxy)-4-methyl-5-(trifluoromethyl)nicotinamido)phenyl)(methyl)(oxo)-λ⁶-sulfaneylidene)amino)-2-oxoethyl)(methyl)carbamate: A mixture of (R)-2-((6-fluoro-2-methylpyridin-3-yl)oxy)-4-methyl-N-(3-(S-methylsulfonimidoyl)phenyl)-5-(trifluoromethyl)nicotinamide (See: WO2022/192487; 120 mg, 0.21 mmol), N-(tert-butoxycarbonyl)-N-methylglycine (80.1 mg, 0.42 mmol) and HATU (118.2 mg, 0.31 mmol) in DMF (3 mL) was added DIEA (80.4 mg, 0.62 mmol) at room temperature. The reaction mixture was stirred at room temperature for 4 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to give tert-butyl (R)-(2-(((3-(2-((6-fluoro-2-methylpyridin-3-yl)oxy)-4-methyl-5-(trifluoromethyl)nicotinamido)phenyl)(methyl)(oxo)-λ⁶-sulfaneylidene)amino)-2-oxoethyl)(methyl)carbamate (110 mg, 80.1%) as a yellow solid. LCMS (ESI) calcd. for C₂₉H₃₀F₄N₅O₆S [M−H]⁻ m/z 652.18, found 652.05.

Step 2: (R)-2-((6-fluoro-2-methylpyridin-3-yl)oxy)-4-methyl-N-(3-(S-methyl-N-(methylglycyl)sulfonimidoyl)phenyl)-5-(trifluoromethyl)nicotinamide: A solution of tert-butyl (R)-(2-(((3-(2-((6-fluoro-2-methylpyridin-3-yl)oxy)-4-methyl-5-(trifluoromethyl)nicotinamido) phenyl)(methyl)(oxo)-λ⁶-sulfaneylidene)amino)-2-oxoethyl)(methyl)carbamate (110 mg, 0.17 mmol) in DCM (3 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% NH₃) to provide (R)-2-((6-fluoro-2-methylpyridin-3-yl)oxy)-4-methyl-N-(3-(S-methyl-N-(methylglycyl)sulfonimidoyl)phenyl)-5-(trifluoromethyl)nicotinamide (51.3 mg, 55.1%) as a white solid. ¹H NMR (400 MHz, DMSO-d₆) δ 11.35 (s, 1H), 8.56 (s, 1H), 8.47 (s, 1H), 8.00-7.79 (m, 2H), 7.78-7.63 (m, 2H), 7.12 (dd, J=8.6, 3.2 Hz, 1H), 3.46 (s, 3H), 3.39 (s, 3H), 3.19 (s, 2H), 2.31-2.18 (m, 6H). LCMS (ESI) calcd. for C₂₄H₂₄F₄N₅O₄S [M+H]⁺ m/z 554.15, found 554.10.

### Example 2D

### (R)-2-((6-fluoro-2-methylpyridin-3-yl)oxy)-N-(3-(N-glycyl-S-methylsulfonimidoyl)phenyl)-4-methyl-5-(trifluoromethyl)nicotinamide

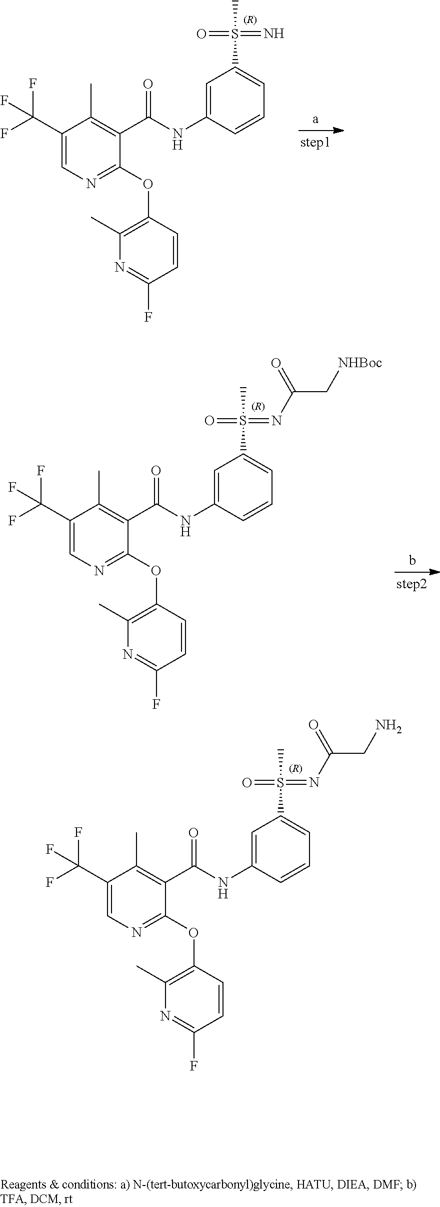

Step 1: tert-butyl (R)-(2-(((3-(2-((6-fluoro-2-methylpyridin-3-yl)oxy)-4-methyl-5-(trifluoromethyl)nicotinamido)phenyl)(methyl)(oxo)-λ⁶-sulfaneylidene)amino)-2-oxoethyl)carbamate: A mixture of (R)-2-((6-fluoro-2-methylpyridin-3-yl)oxy)-4-methyl-N-(3-(S-methylsulfonimidoyl)phenyl)-5-(trifluoromethyl)nicotinamide (100 mg, 0.21 mmol) and (tert-butoxycarbonyl)glycine (44 mg, 0.25 mmol) in DMF (5 mL) was added HATU (122 mg, 0.32 mmol) and DIEA (54 mg, 0.42 mmol). The mixture was stirred at room temperature for 4 hours. The reaction was monitored by LCMS. After the reaction was completed, the resulting solution was diluted with water (50 mL) and extracted with DCM (20 mL×3). The combined organic layers were washed with brine, dried over Na₂SO₄, concentrated under vacuum. The residue was purified by flash column chromatography on silica gel (PE/EtOAc=2/1 to 1/1) to give tert-butyl (R)-(2-(((3-(2-((6-fluoro-2-methylpyridin-3-yl)oxy)-4-methyl-5-(trifluoromethyl)nicotinamido)phenyl)(methyl)(oxo)-λ⁶-sulfaneylidene)amino)-2-oxoethyl) carbamate (100 mg, 74.4%) as a yellow solid. LCMS (ESI) calcd. for C₂₈H₃₀F₄N₅O₆S [M+H]⁺ m/z 640.19, found 640.05.

Step 2: (R)-2-((6-fluoro-2-methylpyridin-3-yl)oxy)-N-(3-(N-glycyl-S-methylsulfonimidoyl)phenyl)-4-methyl-5-(trifluoromethyl)nicotinamide: A solution of tert-butyl (R)-(2-(((3-(2-((6-fluoro-2-methylpyridin-3-yl)oxy)-4-methyl-5-(trifluoromethyl)nicotinamido)phenyl)(methyl)(oxo)-λ⁶-sulfaneylidene)amino)-2-oxoethyl)carbamate (100 mg, 0.15 mmol) in DCM (5 mL) was added TFA (0.5 mL) at room temperature. The reaction mixture was stirred at room temperature for 2 hours. After the reaction was completed, the solution was diluted with water (10 mL) and extracted with DCM (20 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.05% NH₃) to give (R)-2-((6-fluoro-2-methylpyridin-3-yl)oxy)-N-(3-(N-glycyl-S-methylsulfonimidoyl)phenyl)-4-methyl-5-(trifluoromethyl)nicotinamide (36.6 mg, 45.2%) as a white solid. ¹H NMR (400 MHz, CD₃OD-d₄) δ 8.55 (t, J=1.9 Hz, 1H), 8.45 (s, 1H), 8.02-7.95 (m, 1H), 7.83 (d, J=8.7 Hz, 1H), 7.79-7.68 (m, 2H), 6.99 (dd, J=8.6, 3.2 Hz, 1H), 3.46 (d, J=9.1 Hz, 5H), 2.60 (s, 3H), 2.32 (s, 3H). LCMS (ESI) calcd. for C₂₃H₂₂F₄N₅O₄S [M+H]⁺ m/z 540.14, found 540.05.

### Example 3D

### (R)—N-(3-(N-acetyl-S-methylsulfonimidoyl)phenyl)-2-((6-fluoro-2-methylpyridin-3-yl)oxy)-4-methyl-5-(trifluoromethyl)nicotinamide

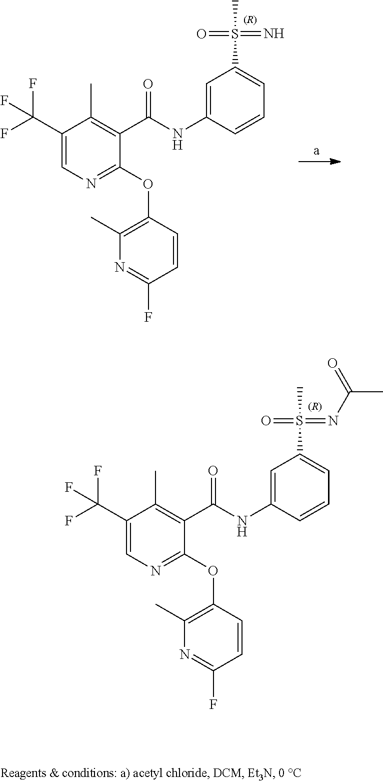

To a mixture of (R)-2-((6-fluoro-2-methylpyridin-3-yl)oxy)-4-methyl-N-(3-(S-methylsulfonimidoyl)phenyl)-5-(trifluoromethyl)nicotinamide (70 mg, 0.15 mmol) and TEA (45.4 mg, 0.45 mmol) in DCM (3 mL) was added a solution of acetyl chloride (23.5 mg, 0.3 mmol) in DCM (1 mL) at 0° C. The reaction mixture was stirred at room temperature for 1 hours. After the reaction was completed, the mixture was adjusted to pH=8-9 with saturated aqueous NaHCO₃. The resulting solution was extracted with DCM (50 mL×3). The combined organic phases were washed with brine, dried over sodium sulfate, concentrated under vacuum. The residue was purified by prep-HPLC (Gemini 5 um C₁₈ column, 150*21.2 mm, eluting with 40% to 95% MeCN/H₂O containing 0.05% NH₄OH) to provide (R)—N-(3-(N-acetyl-S-methylsulfonimidoyl)phenyl)-2-((6-fluoro-2-methylpyridin-3-yl)oxy)-4-methyl-5-(trifluoromethyl)nicotinamide (15.6 mg, 19.8%) as a white solid. ¹H NMR (400 MHz, DMSO-d₆) δ 11.30 (s, 1H), 8.56 (s, 1H), 8.42 (d, J=1.1 Hz, 1H), 7.99-7.80 (m, 2H), 7.73-7.65 (m, 2H), 7.12 (dd, J=8.7, 3.4 Hz, 1H), 3.43 (s, 3H), 2.51 (d, J=1.7 Hz, 3H), 2.25 (s, 3H), 1.98 (s, 3H). LCMS (ESI) calcd. for C₂₃H₂₁F₄N₄O₄S [M+H]⁺ m/z 525.12, found 525.05.

### Example 4D

### (R)—N-(3-(N-(azetidine-3-carbonyl)-S-methylsulfonimidoyl)phenyl)-2-((6-fluoro-2-methylpyridin-3-yl)oxy)-4-methyl-5-(trifluoromethyl)nicotinamide

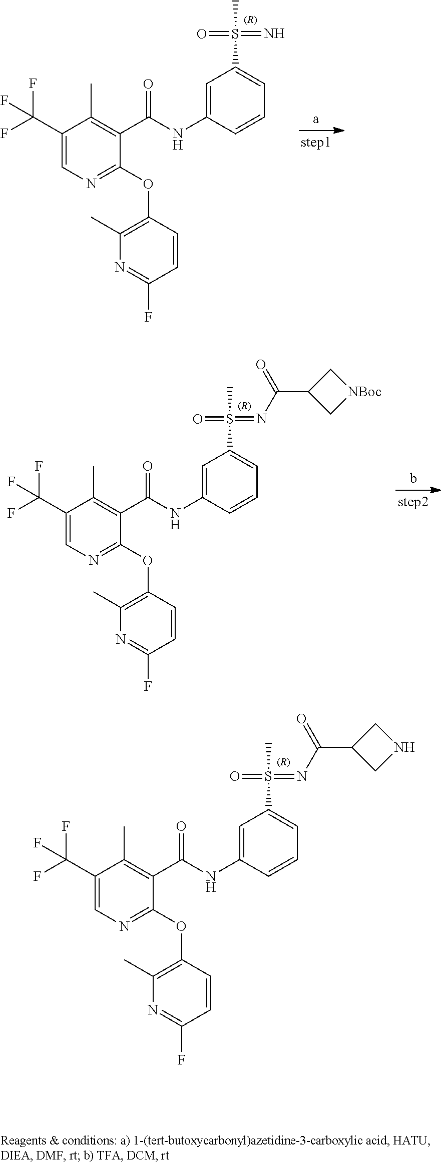

Step 1: tert-butyl (R)-3-(((3-(2-((6-fluoro-2-methylpyridin-3-yl)oxy)-4-methyl-5-(trifluoromethyl)nicotinamido)phenyl)(methyl)(oxo)-λ⁶-sulfaneylidene)carbamoyl)azetidine-1-carboxylate: To a solution of (R)-2-((6-fluoro-2-methylpyridin-3-yl)oxy)-4-methyl-N-(3-(S-methylsulfonimidoyl)phenyl)-5-(trifluoromethyl)nicotinamide (100 mg, 0.21 mmol), 1-(tert-butoxycarbonyl)azetidine-3-carboxylic acid (84 mg, 0.42 mmol) in DMF (3 mL) was added DIEA (81 mg, 0.63 mmol) and HATU (118 mg, 0.31 mmol). Then the mixture was heated at 25° C. for 3 hours. LCMS showed the rection was completed. The mixture was diluted with water and extracted with EtOAc (3×30 mL). The combined organic phases were washed with brine, dried over Na₂SO₄, concentrated under vacuum. The residue was purified by silica gel column chromatography (eluting with MeOH/DCM, 0 to 10%) to give tert-butyl (R)-3-(((3-(2-((6-fluoro-2-methylpyridin-3-yl)oxy)-4-methyl-5-(trifluoromethyl)nicotinamido)phenyl)(methyl)(oxo)-λ⁶-sulfaneylidene)carbamoyl)azetidine-1-carboxylate (110 mg, 78.8%) as a yellow oil. LCMS (ESI) calcd. for C₃₀H₃₂F₄N₅O₆S [M+H]⁺ m/z 666.20, found 666.10.

Step 2: (R)—N-(3-(N-(azetidine-3-carbonyl)-S-methylsulfonimidoyl)phenyl)-2-((6-fluoro-2-methylpyridin-3-yl)oxy)-4-methyl-5-(trifluoromethyl)nicotinamide: A solution of tert-butyl (R)-3-(((3-(2-((6-fluoro-2-methylpyridin-3-yl)oxy)-4-methyl-5-(trifluoromethyl)nicotinamido)phenyl)(methyl)(oxo)-λ⁶-sulfaneylidene)carbamoyl)azetidine-1-carboxylate (110 mg, 0.17 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (30 mL×3). The combined organic phases were washed with brine, dried over sodium sulfate, concentrated under vacuum. The residue was purified by prep-HPLC (Xbridge 5 um C₁₈ column, 150*19 mm, eluting with 25% to 75% ACN/H₂O containing 0.05% NH₃) to provide (R)—N-(3-(N-(azetidine-3-carbonyl)-S-methylsulfonimidoyl)phenyl)-2-((6-fluoro-2-methylpyridin-3-yl)oxy)-4-methyl-5-(trifluoromethyl)nicotinamide (28.16 mg, 29.32%) as a white solid. ¹H NMR (400 MHz, CD₃OD) δ 8.71 (s, 1H), 8.49 (d, J=27.9 Hz, 2H), 7.85 (d, J=6.0 Hz, 2H), 7.79-7.71 (m, 2H), 6.99 (dd, J=8.8, 2.7 Hz, 1H), 4.26 (d, J=8.3 Hz, 4H), 3.83-3.68 (m, 1H), 3.52 (s, 3H), 2.60 (s, 3H), 2.31 (s, 3H). LCMS (ESI) calcd. for C₂₅H₂₄F₄N₅O₄S [M+H]⁺ m/z 566.15, found 565.90.

### Example 5D

### (R)—N-(3-(N-(1-aminocyclopropane-1-carbonyl)-S-methylsulfonimidoyl)phenyl)-2-((6-fluoro-2-methylpyridin-3-yl)oxy)-4-methyl-5-(trifluoromethyl)nicotinamide

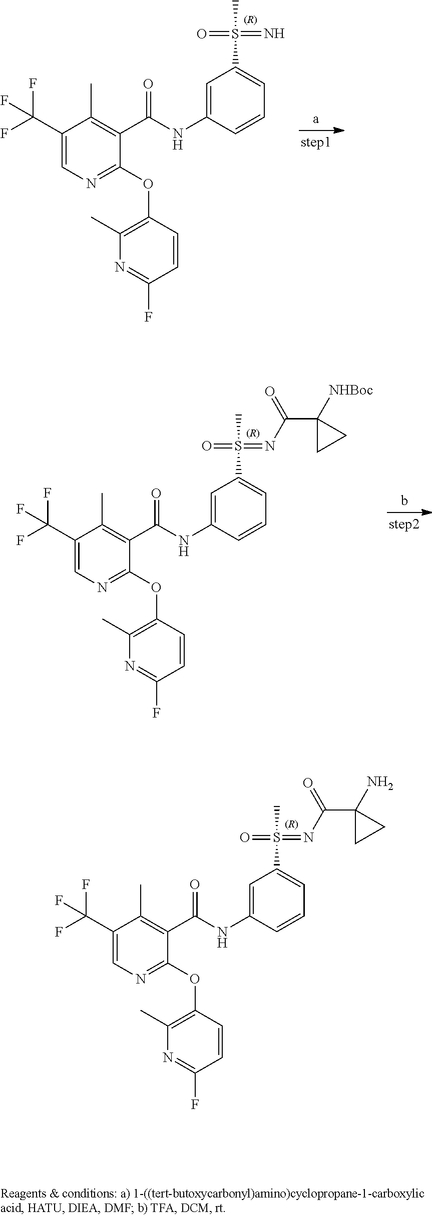

Step 1: tert-butyl (R)-(1-(((3-(2-((6-fluoro-2-methylpyridin-3-yl)oxy)-4-methyl-5-(trifluoromethyl)nicotinamido)phenyl)(methyl)(oxo)-λ⁶-sulfaneylidene)carbamoyl)cyclopropyl)carbamate: A mixture of (R)-2-((6-fluoro-2-methylpyridin-3-yl)oxy)-4-methyl-N-(3-(S-methylsulfonimidoyl)phenyl)-5-(trifluoromethyl)nicotinamide (120 mg, 0.25 mmol), 1-((tert-butoxycarbonyl)amino)cyclopropane-1-carboxylic acid (100 mg, 0.50 mmol) and HATU (141.8 mg, 0.37 mmol) in DMF (5 mL) was added DIEA (96.4 mg, 0.75 mmol) at room temperature. The reaction mixture was heated at 50° C. for 16 hours. After the reaction was completed, the resulting solution was diluted with water (40 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to give tert-butyl (R)-(1-(((3-(2-((6-fluoro-2-methylpyridin-3-yl)oxy)-4-methyl-5-(trifluoromethyl)nicotinamido)phenyl)(methyl) (oxo)-λ⁶-sulfaneylidene)carbamoyl)cyclopropyl)carbamate (100 mg, 60.3%) as a yellow solid. LCMS (ESI) calcd. for C₃₀H₃₂F₄N₅O₆S [M+H]⁺ m/z 666.20, found 666.15.

Step 2: (R)—N-(3-(N-(1-aminocyclopropane-1-carbonyl)-S-methylsulfonimidoyl)phenyl)-2-((6-fluoro-2-methylpyridin-3-yl)oxy)-4-methyl-5-(trifluoromethyl)nicotinamide. A solution of tert-butyl (R)-(1-(((3-(2-((6-fluoro-2-methylpyridin-3-yl)oxy)-4-methyl-5-(trifluoromethyl)nicotinamido)phenyl)(methyl)(oxo)-λ⁶-sulfaneylidene)carbamoyl)cyclopropyl) carbamate (100 mg, 0.15 mmol) in DCM (3 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% NH₃) to provide (R)—N-(3-(N-(1-aminocyclopropane-1-carbonyl)-S-methylsulfonimidoyl)phenyl)-2-((6-fluoro-2-methylpyridin-3-yl)oxy)-4-methyl-5-(trifluoromethyl)nicotinamide (52.8 mg, 62.2%) as a white solid. ¹H NMR (400 MHz, DMSO-d₆) δ 11.30 (s, 1H), 8.56 (s, 1H), 8.44 (s, 1H), 8.00-7.80 (m, 2H), 7.69 (d, J=6.3 Hz, 2H), 7.12 (dd, J=8.6, 3.3 Hz, 1H), 3.44 (s, 3H), 2.51 (s, 3H), 2.25 (s, 3H), 2.20-2.03 (m, 2H), 1.28-1.12 (m, 2H), 0.85-0.72 (m, 2H). LCMS (ESI) calcd. for C₂₅H₂₄F₄N₅O₄S [M+H]⁺ m/z 566.15, found 566.15.

### Example 6D

### (R)-2-((6-fluoro-2-methylpyridin-3-yl)oxy)-4-methyl-N-(3-(S-methyl-N-(oxetane-3-carbonyl)sulfonimidoyl)phenyl)-5-(trifluoromethyl)nicotinamide

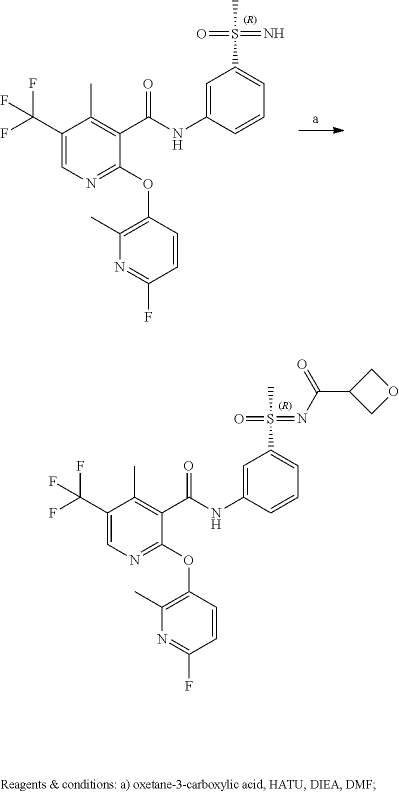

To a mixture of (R)-2-((6-fluoro-2-methylpyridin-3-yl)oxy)-4-methyl-N-(3-(S-methylsulfonimidoyl)phenyl)-5-(trifluoromethyl)nicotinamide (100 mg, 0.20 mmol), oxetane-3-carboxylic acid (32 mg, 0.25 mmol) and HATU (152 mg, 0.40 mmol) in DMF (5 mL) was added DIEA (78 mg, 0.60 mmol). The mixture was stirred at room temperature for 2 hours. After the reaction was completed, the resulting solution was diluted with water (50 mL) and extracted with EtOAc (30 mL×3). The combined organic phases were washed with brine, dried over Na₂SO₄, concentrated under vacuum. The residue was purified by flash column chromatography on silica gel (PE/EtOAc=1/4) to give the crude product. The residue was purified by prep-HPLC (Gemini um C₁₈ column, 150*21.2 mm, eluting with 50% to 95% MeCN/H₂O containing 0.1% formic acid) to provide (R)-2-((6-fluoro-2-methylpyridin-3-yl)oxy)-4-methyl-N-(3-(S-methyl-N-(oxetane-3-carbonyl)sulfonimidoyl)phenyl)-5-(trifluoromethyl)nicotinamide (39.2 mg, 33.3%) as a white solid. ¹H NMR (400 MHz, DMSO-d₆) δ 11.32 (s, 1H), 8.56 (s, 1H), 8.48 (s, 1H), 7.93 (d, J=6.9 Hz, 1H), 7.88-7.81 (m, 1H), 7.74-7.62 (m, 2H), 7.11 (dd, J=8.6, 3.6 Hz, 1H), 4.73-4.45 (m, 4H), 3.94-3.67 (m, 1H), 3.49 (s, 3H), 2.42 (m, 3H), 2.24 (s, 3H). LCMS (ESI) calcd. for C₂₅H₂₃F₄N₄O₅S [M+H]⁺ m/z 567.13, found 567.05.

### Example 7D

### 2-((6-fluoro-2-methylpyridin-3-yl)oxy)-4-methyl-N-(3-((R)—S-methyl-N—((S)-tetrahydrofuran-2-carbonyl)sulfonimidoyl)phenyl)-5-(trifluoromethyl)nicotinamide

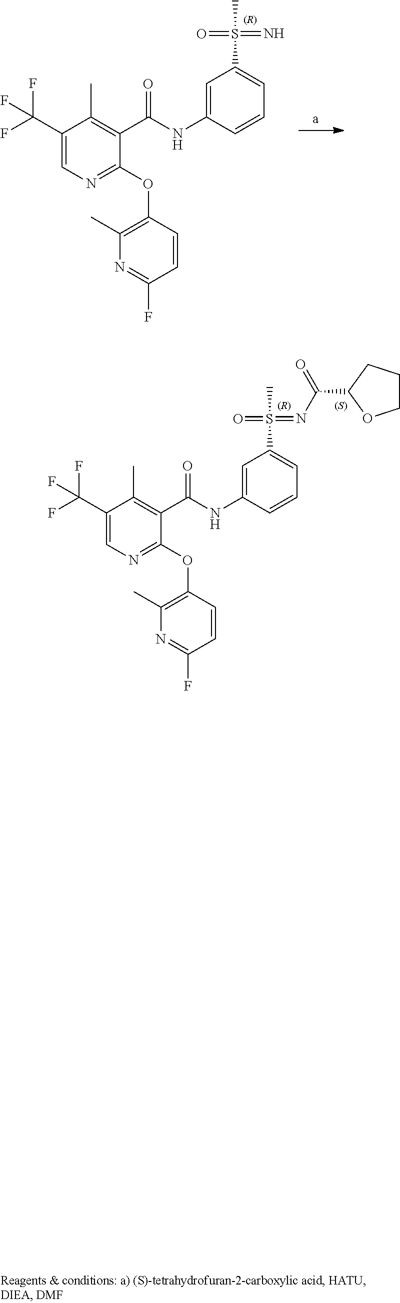

A solution of (R)-2-((6-fluoro-2-methylpyridin-3-yl)oxy)-4-methyl-N-(3-(S-methylsulfonimidoyl)phenyl)-5-(trifluoromethyl)nicotinamide (110 mg, 0.22 mmol), (S)-tetrahydrofuran-2-carboxylic acid (53 mg, 0.46 mmol), HATU (173 mg, 0.46 mmol) and DIEA (88 mg, 0.68 mmol) in DMF (5 mL) was stirred at room temperature for 16 hours. LCMS showed the reaction was completed. The mixture was diluted with water (60 mL) and extracted with EtOAc (30 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% NH₃H₂O) to provide 2-((6-fluoro-2-methylpyridin-3-yl)oxy)-4-methyl-N-(3-((R)—S-methyl-N—((S)-tetrahydrofuran-2-carbonyl)sulfonimidoyl)phenyl)-5-(trifluoromethyl)nicotinamide (53.9 mg, 44.7%) as a white solid. ¹H NMR (400 MHz, DMSO-d₆) δ 11.32 (s, 1H), 8.52 (d, J=27.3 Hz, 2H), 7.95-7.78 (m, 2H), 7.70 (d, J=4.8 Hz, 2H), 7.12 (dd, J=8.6, 3.3 Hz, 1H), 4.29 (dd, J=8.3, 5.3 Hz, 1H), 3.83-3.65 (m, 2H), 3.46 (s, 3H), 2.50 (s, 3H), 2.24 (s, 3H), 2.18-2.06 (m, 1H), 1.99-1.85 (m, 1H), 1.82-1.68 (m, 2H). LCMS (ESI) calcd. for C₂₆H₂₅F₄N₄O₅S [M+H]⁺ m/z 581.15, found 581.10.

### Example 8D

### (R)-2-((6-fluoro-2-methylpyridin-3-yl)oxy)-N-(3-(N-(2-methoxyacetyl)-S-methylsulfonimidoyl)phenyl)-4-methyl-5-(trifluoromethyl)nicotinamide

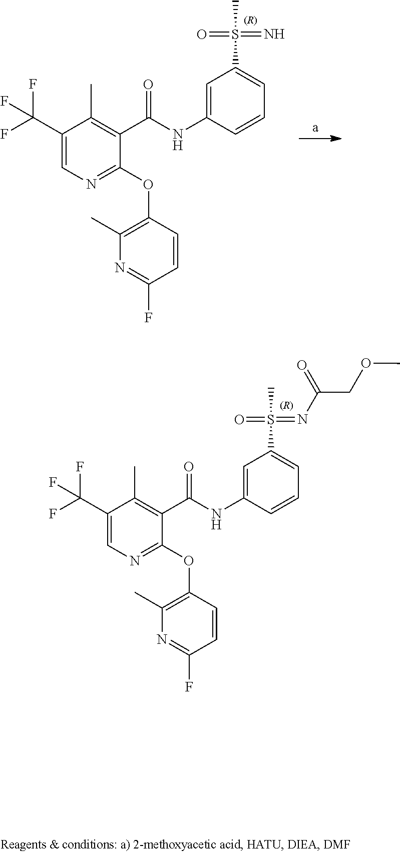

A solution of (R)-2-((6-fluoro-2-methylpyridin-3-yl)oxy)-4-methyl-N-(3-(S-methylsulfonimidoyl)phenyl)-5-(trifluoromethyl)nicotinamide (110 mg, 0.22 mmol), 2-methoxyacetic acid (41 mg, 0.46 mmol), HATU (173 mg, 0.46 mmol) and DIEA (88 mg, 0.68 mmol) in DMF (5 mL) was stirred at room temperature for 16 hours. LCMS showed the reaction was completed. The mixture was diluted with water (60 mL) and extracted with EtOAc (30 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% NH₃·H₂O) to provide (R)-2-((6-fluoro-2-methylpyridin-3-yl)oxy)-N-(3-(N-(2-methoxyacetyl)-S-methylsulfonimidoyl)phenyl)-4-methyl-5-(trifluoromethyl)nicotinamide (54.2 mg, 46.7%) as a white solid. ¹H NMR (400 MHz, DMSO-d₆) δ 11.32 (s, 1H), 8.56 (s, 1H), 8.46 (s, 1H), 7.94 (dt, J=6.6, 2.1 Hz, 1H), 7.84 (dd, J=8.6, 7.0 Hz, 1H), 7.74-7.66 (m, 2H), 7.11 (dd, J=8.7, 3.3 Hz, 1H), 3.94 (s, 2H), 3.48 (s, 3H), 3.26 (s, 3H), 2.50 (s, 3H), 2.24 (s, 3H). LCMS (ESI) calcd. for C₂₄H₂₃F₄N₄O₅S [M+H]⁺ m/z 555.13, found 555.10.

### Example 9D

### N-(3-((R)—N-(D-alanyl)-S-methylsulfonimidoyl)phenyl)-2-((6-fluoro-2-methylpyridin-3-yl)oxy)-4-methyl-5-(trifluoromethyl)nicotinamide formate

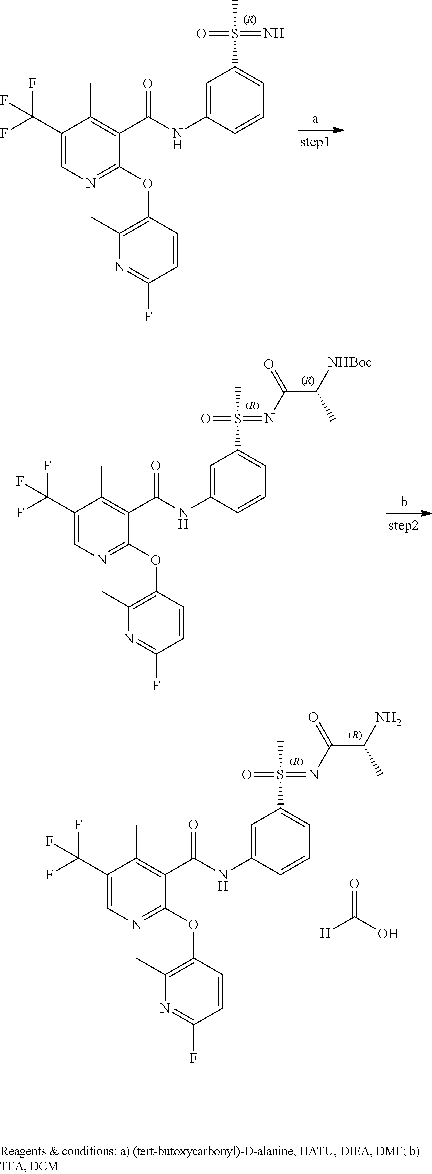

Step 1: tert-butyl ((R)-1-(((R)-(3-(2-((6-fluoro-2-methylpyridin-3-yl)oxy)-4-methyl-5-(trifluoromethyl)nicotinamido)phenyl)(methyl)(oxo)-λ⁶-sulfaneylidene)amino)-1-oxopropan-2-yl)carbamate: To a solution of (R)-2-((6-fluoro-2-methylpyridin-3-yl)oxy)-4-methyl-N-(3-(S-methylsulfonimidoyl)phenyl)-5-(trifluoromethyl)nicotinamide (100 mg, 0.21 mmol), (tert-butoxycarbonyl)-D-alanine (100 mg, 0.53 mmol) and HATU (122 mg, 0.32 mmol) in DMF (3 mL) and was added DIEA (81 mg, 0.63 mmol). Then the mixture was heated at 25° C. for 3 hours. LCMS showed the rection was completed. The mixture was diluted with water and extracted with EtOAc (3×6 mL). The combined organic layers were washed with brine, dried with Na₂SO₄, and concentrated under vacuum. The residue was purified by silica gel column chromatography (eluting with MeOH/DCM, 0 to 10%) to give tert-butyl ((R)-1-(((R)-(3-(2-((6-fluoro-2-methylpyridin-3-yl)oxy)-4-methyl-5-(trifluoromethyl)nicotinamido)phenyl)(methyl)(oxo)-λ⁶-sulfaneylidene)amino)-1-oxopropan-2-yl)carbamate (110 mg, 80.22%) as a yellow oil. LCMS (ESI) calcd. for C₂₉H₃₂F₄N₅O₆S [M+H]⁺ m/z 654.19, found 654.10.

Step 2: N-(3-((R)—N-(D-alanyl)-S-methylsulfonimidoyl)phenyl)-2-((6-fluoro-2-methylpyridin-3-yl)oxy)-4-methyl-5-(trifluoromethyl)nicotinamide formate: A solution of tert-butyl ((R)-1-(((R)-(3-(2-((6-fluoro-2-methylpyridin-3-yl)oxy)-4-methyl-5-(trifluoromethyl) nicotinamido)phenyl)(methyl)(oxo)-λ⁶-sulfaneylidene)amino)-1-oxopropan-2-yl)carbamate (110 mg, 0.17 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (10 mL×3). The combined organic phases were washed with brine, dried over sodium sulfate, concentrated under vacuum. The residue was purified by prep-HPLC (Gemini 5 um C₁₈ column, 150*19 mm, eluting with 20% to 30% ACN/H₂O containing 0.1% formic acid) to provide N-(3-((R)—N-(D-alanyl)-S-methylsulfonimidoyl)phenyl)-2-((6-fluoro-2-methylpyridin-3-yl)oxy)-4-methyl-5-(trifluoromethyl)nicotinamide formate (32.50 mg, 34.57%) as a white solid. ¹H NMR (400 MHz, CD₃OD) δ 8.71 (d, J=1.9 Hz, 1H), 8.54 (s, 1H), 8.46 (s, 1H), 7.93-7.81 (m, 2H), 7.79-7.68 (m, 2H), 6.99 (dd, J=8.6, 3.0 Hz, 1H), 3.94 (dd, J=14.3, 7.3 Hz, 1H), 3.53 (d, J=10.4 Hz, 3H), 2.60 (s, 3H), 2.31 (s, 3H), 1.59 (dd, J=7.2, 4.5 Hz, 3H). LCMS (ESI) calcd. for C₂₄H₂₄F₄N₅O₄S [M+H]⁺ m/z 554.14, found 553.95.

### Example 10D

### 2-((6-fluoro-2-methylpyridin-3-yl)oxy)-4-methyl-N-(3-((R)—S-methyl-N—((R)-tetrahydrofuran-3-carbonyl)sulfonimidoyl)phenyl)-5-(trifluoromethyl)nicotinamide

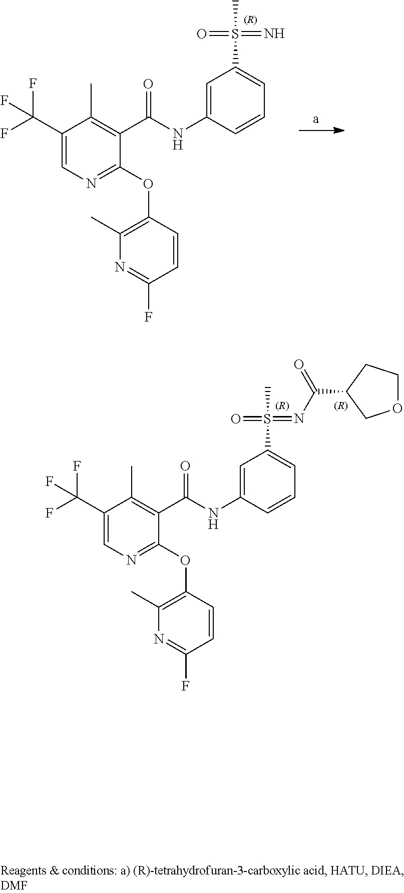

A mixture of (R)-2-((6-fluoro-2-methylpyridin-3-yl)oxy)-4-methyl-N-(3-(S-methylsulfonimidoyl)phenyl)-5-(trifluoromethyl)nicotinamide (100 mg, 0.20 mmol), (R)-tetrahydrofuran-3-carboxylic acid (36 mg, 0.31 mmol), HATU (158 mg, 0.41 mmol) and DIEA (81 mg, 0.62 mmol) in DMF (3 mL) was stirred at room temperature for 2 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by prep-HPLC (Gemini 5 um C₁₈ column, 150*21.2 mm, eluting with 50% to 95% MeCN/H₂O containing 0.05% NH₃) to provide 2-((6-fluoro-2-methylpyridin-3-yl)oxy)-4-methyl-N-(3-((R)—S-methyl-N—((R)-tetrahydrofuran-3-carbonyl)sulfonimidoyl)phenyl)-5-(trifluoromethyl)nicotinamide (51.25 mg, 60.75%) as a white solid. ¹H NMR (400 MHz, DMSO-d₆) δ 11.31 (s, 1H), 8.56 (s, 1H), 8.47 (s, 1H), 7.94-7.87 (m, 1H), 7.84 (dd, J=8.7, 6.8 Hz, 1H), 7.70 (s, 2H), 7.11 (dd, J=8.7, 3.1 Hz, 1H), 3.80 (dq, J=8.5, 7.3, 6.2 Hz, 2H), 3.68-3.58 (m, 2H), 3.46 (s, 3H), 3.05 (tt, J=8.6, 6.3 Hz, 1H), 2.51 (s, 3H), 2.24 (s, 3H), 2.09-1.94 (m, 2H). LCMS (ESI) calcd. for C₂₆H₂₅F₄N₄O₅S [M+H]⁺ m/z 581.15, found 580.90.

### Example 11D

### N-(3-((R)—N-(2-amino-3,3,3-trifluoropropanoyl)-S-methylsulfonimidoyl)phenyl)-2-((6-fluoro-2-methylpyridin-3-yl)oxy)-4-methyl-5-(trifluoromethyl)nicotinamide

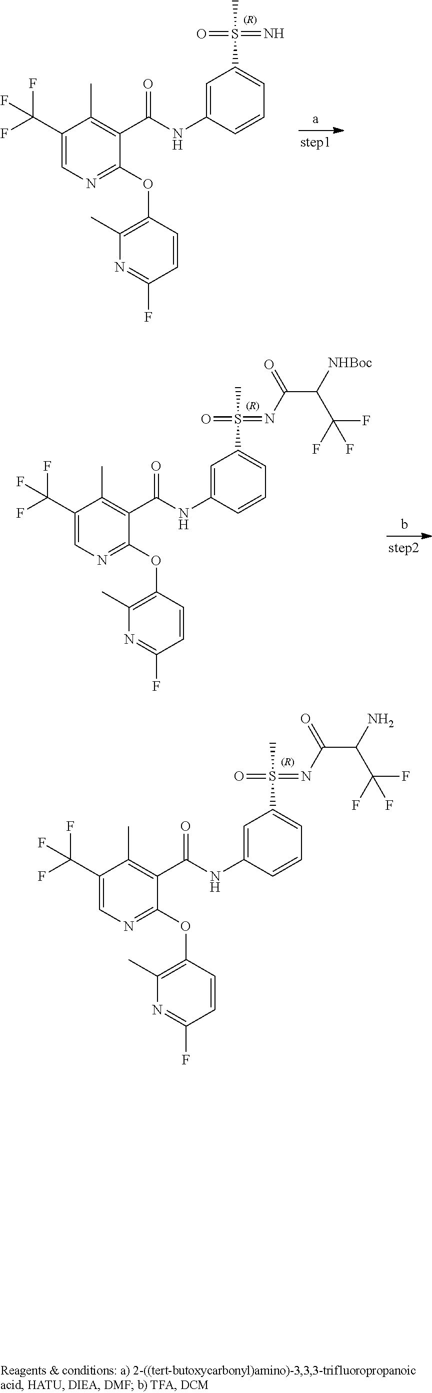

Step 1: tert-butyl (1,1,1-trifluoro-3-(((R)-(3-(2-((6-fluoro-2-methylpyridin-3-yl)oxy)-4-methyl-5-(trifluoromethyl)nicotinamido)phenyl)(methyl)(oxo)-λ⁶-sulfaneylidene)amino)-3-oxopropan-2-yl)carbamate: To a solution of (R)-2-((6-fluoro-2-methylpyridin-3-yl)oxy)-4-methyl-N-(3-(S-methylsulfonimidoyl)phenyl)-5-(trifluoromethyl)nicotinamide (150 mg, 0.31 mmol), 2-((tert-butoxycarbonyl)amino)-3,3,3-trifluoropropanoic acid (113 mg, 0.47 mmol) and HATU (235 mg, 0.62 mmol) in DMF (3 mL) and was added DIEA (120 mg, 0.93 mmol). Then the mixture was stirred at 25° C. for 3 hours. LCMS showed the rection was completed. The mixture was diluted with water (20 mL) and extracted with EtOAc (3×20 mL). The combined organic layers were washed with brine, dried with Na₂SO₄, and concentrated under vacuum. The residue was purified by flash column chromatography on silica gel (PE/EtOAc=1/4) to provide tert-butyl (1,1,1-trifluoro-3-(((R)-(3-(2-((6-fluoro-2-methylpyridin-3-yl)oxy)-4-methyl-5-(trifluoromethyl)nicotinamido)phenyl)(methyl)(oxo)-λ⁶-sulfaneylidene)amino)-3-oxopropan-2-yl)carbamate (110 mg, 49.99%) as a yellow oil. LCMS (ESI) calcd. for C₂₉H₂₉F₇N₅O₆S [M+H]⁺ m/z 708.17, found 708.10.

Step 2: N-(3-((R)—N-(2-amino-3,3,3-trifluoropropanoyl)-S-methylsulfonimidoyl)phenyl)-2-((6-fluoro-2-methylpyridin-3-yl)oxy)-4-methyl-5-(trifluoromethyl)nicotinamide: A solution of tert-butyl (1,1,1-trifluoro-3-(((R)-(3-(2-((6-fluoro-2-methylpyridin-3-yl)oxy)-4-methyl-5-(trifluoromethyl)nicotinamido)phenyl)(methyl)(oxo)-λ⁶-sulfaneylidene)amino)-3-oxopropan-2-yl)carbamate (110 mg, 0.16 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (10 mL×3). The combined organic phases were washed with brine, dried over sodium sulfate, concentrated under vacuum. The residue was purified by prep-HPLC (Gemini 5 um C₁₈ column, 150*21.2 mm, eluting with 30% to 90% MeCN/H₂O containing 0.05% NH₃) to provide N-(3-((R)—N-(2-amino-3,3,3-trifluoropropanoyl)-S-methylsulfonimidoyl)phenyl)-2-((6-fluoro-2-methylpyridin-3-yl)oxy)-4-methyl-5-(trifluoromethyl)nicotinamide (60 mg, 63.83%) as a white solid. ¹H NMR (400 MHz, CD₃OD) δ 8.53 (t, J=2.0 Hz, 1H), 8.42 (s, 1H), 7.96 (dd, J=8.6, 7.2 Hz, 1H), 7.80 (dd, J=6.2, 4.6 Hz, 1H), 7.76-7.65 (m, 2H), 6.96 (dd, J=8.7, 3.3 Hz, 1H), 4.06-3.92 (m, 1H), 3.49 (d, J=7.4 Hz, 3H), 2.58 (s, 3H), 2.29 (s, 3H). LCMS (ESI) calcd. for C₂₄H₂₁F₇N₅O₄S [M+H]⁺ m/z 608.12, found 608.00.

### Example 12D

### (R)-2-((6-fluoro-2-methylpyridin-3-yl)oxy)-N-(3-(N-(2-hydroxyacetyl)-S-methylsulfonimidoyl)phenyl)-4-methyl-5-(trifluoromethyl)nicotinamide

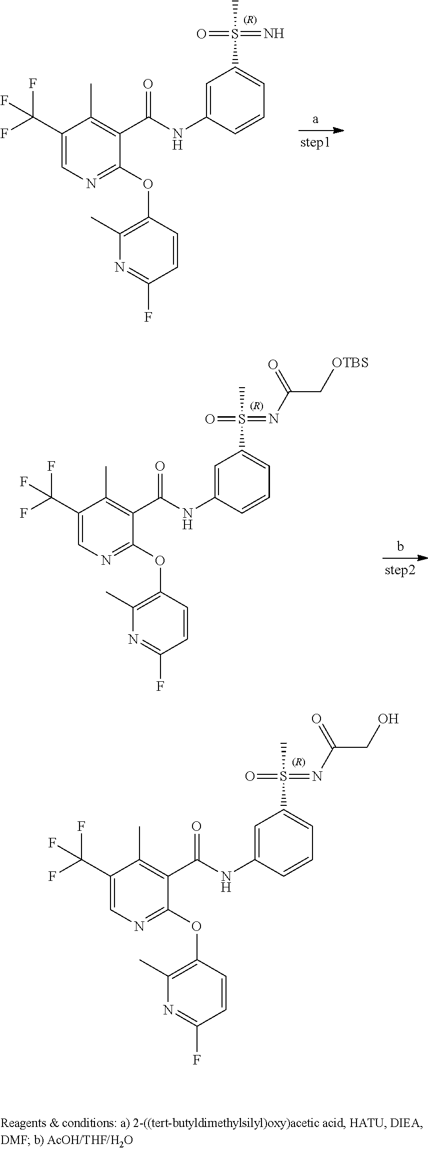

Step 1: (R)—N-(3-(N-(2-((tert-butyldimethylsilyl)oxy)acetyl)-S-methylsulfonimidoyl)phenyl)-2-((6-fluoro-2-methylpyridin-3-yl)oxy)-4-methyl-5-(trifluoromethyl)nicotinamide: To a solution of (R)-2-((6-fluoro-2-methylpyridin-3-yl)oxy)-4-methyl-N-(3-(S-methylsulfonimidoyl)phenyl)-5-(trifluoromethyl)nicotinamide (150 mg, 0.31 mmol), 2-((tert-butyldimethylsilyl)oxy)acetic acid (296 mg, 1.56 mmol) and HATU (236 mg, 0.62 mmol) in DMF (5 mL) and was added DIEA (120 mg, 0.93 mmol). Then the mixture was heated at 25° C. for 2 hours. LCMS showed the rection was completed. The mixture was diluted with water and extracted with EtOAc (3×30 mL). The combined organic layers were washed with brine, dried with Na₂SO₄, and concentrated under vacuum. The residue was purified by flash column chromatography on silica gel (PE/EtOAc=1/1) to give (R)—N-(3-(N-(2-((tert-butyldimethylsilyl)oxy)acetyl)-S-methylsulfonimidoyl)phenyl)-2-((6-fluoro-2-methylpyridin-3-yl)oxy)-4-methyl-5-(trifluoromethyl)nicotinamide (100 mg, 49.14%) as a yellow oil. LCMS (ESI) calcd. for C₃₀H₃₅F₄N₄O₅SSi [M+H]⁺ m/z 655.20, found 655.15.

Step 2: (R)-2-((6-fluoro-2-methylpyridin-3-yl)oxy)-N-(3-(N-(2-hydroxyacetyl)-S-methylsulfonimidoyl)phenyl)-4-methyl-5-(trifluoromethyl)nicotinamide: To a solution of (R)—N-(3-(N-(2-((tert-butyldimethylsilyl)oxy)acetyl)-S-methylsulfonimidoyl)phenyl)-2-((6-fluoro-2-methylpyridin-3-yl)oxy)-4-methyl-5-(trifluoromethyl)nicotinamide (100 mg, 0.15 mmol) in THF (1 mL) was added H₂O (3 mL) and AcOH (3 mL) at room temperature. The reaction mixture was stirred at room temperature for 2 hours. After the reaction was completed, the mixture was diluted with water and extracted with EtOAc (3×30 mL). The combined organic layers were washed with brine, dried with Na₂SO₄, and concentrated under vacuum. The residue was purified by prep-HPLC (Gemini 5 um C₁₈ column, 150*19 mm, eluting with 20% to 30% ACN/120 containing 0.1% formic acid) to provide (R)-2-((6-fluoro-2-methylpyridin-3-yl)oxy)-N-(3-(N-(2-hydroxyacetyl)-S-methylsulfonimidoyl)phenyl)-4-methyl-5-(trifluoromethyl)nicotinamide (60.5 mg, 66.5%) as a white solid. ¹H NMR (400 MHz, DMSO-d₆) δ 11.31 (s, 1H), 8.56 (s, 1H), 8.43 (s, 1H), 7.99-7.81 (m, 2H), 7.75-7.64 (m, 2H), 7.11 (dd, J=8.7, 3.4 Hz, 1H), 4.79 (t, J=6.3 Hz, 1H), 3.95 (d, J=6.3 Hz, 2H), 3.47 (s, 3H), 2.51 (s, 3H), 2.25 (s, 3H). LCMS (ESI) calcd. for C₂₃H₂₁F₄N₄O₅S [M+H]⁺ m/z 541.11, found 541.00.

### Example 13D

### 2-((6-fluoro-2-methylpyridin-3-yl)oxy)-N-(3-((R)—N—((S)-2-hydroxypropanoyl)-S-methylsulfonimidoyl)phenyl)-4-methyl-5-(trifluoromethyl)nicotinamide

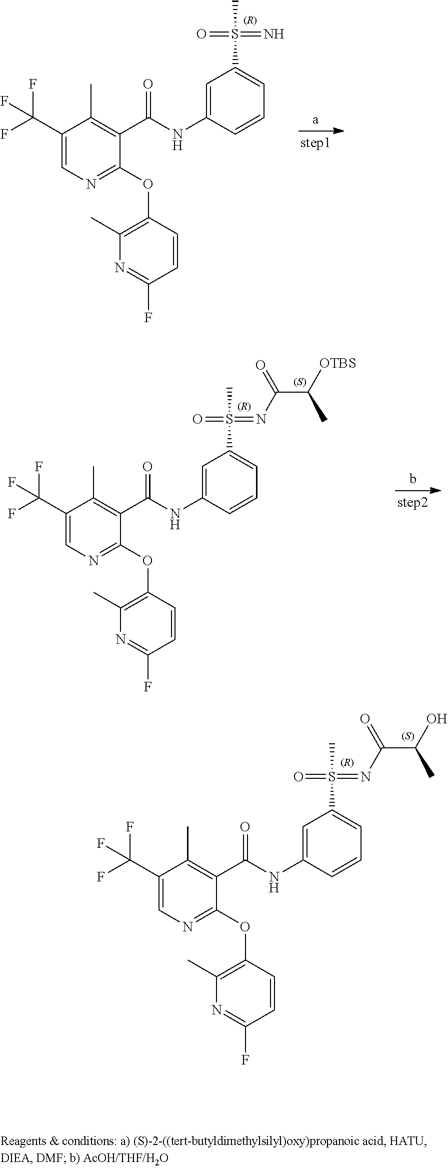

Step 1: N-(3-((R)—N—((S)-2-((tert-butyldimethylsilyl)oxy)propanoyl)-S-methylsulfonimidoyl)phenyl)-2-((6-fluoro-2-methylpyridin-3-yl)oxy)-4-methyl-5-(trifluoromethyl)nicotinamide: To a solution of (R)-2-((6-fluoro-2-methylpyridin-3-yl)oxy)-4-methyl-N-(3-(S-methylsulfonimidoyl)phenyl)-5-(trifluoromethyl)nicotinamide (150 mg, 0.31 mmol), (S)-2-((tert-butyldimethylsilyl)oxy)propanoic acid (317 mg, 1.56 mmol) and HATU (236 mg, 0.62 mmol) in DMF (5 mL) and was added DIEA (120 mg, 0.93 mmol). Then the mixture was stirred at 25° C. for 2 hours. LCMS showed the rection was completed. The mixture was diluted with water and extracted with EtOAc (3×30 mL). The combined organic layers were washed with brine, dried with Na₂SO₄, and concentrated under vacuum. The residue was purified by flash column chromatography on silica gel (PE/EtOAc=1/1) to give N-(3-((R)—N—((S)-2-((tert-butyldimethylsilyl)oxy)propanoyl)-S-methylsulfonimidoyl)phenyl)-2-((6-fluoro-2-methylpyridin-3-yl)oxy)-4-methyl-5-(trifluoromethyl)nicotinamide (110 mg, 52.9%) as a yellow oil. LCMS (ESI) calcd. for C₃₀H₃₇F₄N₄O₅SSi [M+H]⁺ m/z 669.22, found 669.15.

Step 2: 2-((6-fluoro-2-methylpyridin-3-yl)oxy)-N-(3-((R)—N—((S)-2-hydroxypropanoyl)-S-methylsulfonimidoyl)phenyl)-4-methyl-5-(trifluoromethyl)nicotinamide: To a solution of N-(3-((R)—N—((S)-2-((tert-butyldimethylsilyl)oxy)propanoyl)-S-methylsulfonimidoyl)phenyl)-2-((6-fluoro-2-methylpyridin-3-yl)oxy)-4-methyl-5-(trifluoromethyl)nicotinamide (110 mg, 0.16 mmol) in THE (1 mL) was added H₂O (3 mL) and AcOH (3 mL) at room temperature. The reaction mixture was stirred at room temperature for 2 hours. After the reaction was completed, the mixture was diluted with water and extracted with EtOAc (3×30 mL). The combined organic layers were washed with brine, dried with Na₂SO₄, and concentrated under vacuum. The residue was purified by prep-HPLC (Gemini 5 um C₁₈ column, 150*19 mm, eluting with 20% to 30% ACN/H₂O containing 0.1% formic acid) to provide 2-((6-fluoro-2-methylpyridin-3-yl)oxy)-N-(3-((R)—N—((S)-2-hydroxypropanoyl)-S-methylsulfonimidoyl)phenyl)-4-methyl-5-(trifluoromethyl)nicotinamide (60.5 mg, 66.48%) as a white solid. ¹H NMR (400 MHz, DMSO-d₆) δ 11.31 (s, 1H), 8.56 (s, 1H), 8.47 (s, 1H), 7.95-7.81 (m, 2H), 7.74-7.66 (m, 2H), 7.11 (dd, J=8.6, 3.3 Hz, 1H), 4.70 (d, J=5.6 Hz, 1H), 4.11-3.96 (m, 1H), 3.46 (s, 3H), 2.50 (s, 3H), 2.24 (s, 3H), 1.25 (d, J=6.8 Hz, 3H). LCMS (ESI) calcd. for C₂₄H₂₃F₄N₄O₅S [M+H]⁺ m/z 555.13, found 555.05.

### Example 14D

### 2-((6-fluoro-2-methylpyridin-3-yl)oxy)-4-methyl-N-(3-((R)—S-methyl-N—((R)-tetrahydrofuran-2-carbonyl)sulfonimidoyl)phenyl)-5-(trifluoromethyl)nicotinamide

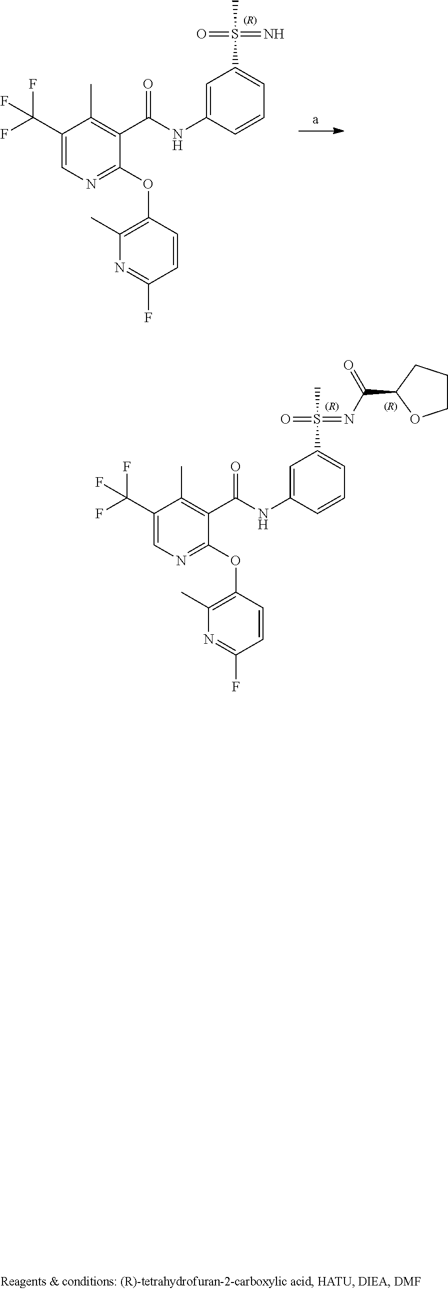

A mixture of (R)-2-((6-fluoro-2-methylpyridin-3-yl)oxy)-4-methyl-N-(3-(S-methylsulfonimidoyl)phenyl)-5-(trifluoromethyl)nicotinamide (120 mg, 0.25 mmol), (R)-tetrahydrofuran-2-carboxylic acid (57.8 mg, 0.5 mmol) and HATU (141.8 mg, 0.37 mmol) in DMF (5 mL) was added DIEA (96.4 mg, 0.75 mmol) at room temperature. The reaction mixture was stirred at room temperature for 4 hours. After the reaction was completed, the solution was diluted with water (30 mL) and extracted with EtOAc (30 mL×3). The combined organic layers were washed with brine, dried over sodium sulfate, concentrated under vacuum. The residue was purified by prep-HPLC (Gemini 5 um C₁₈ column, 150*21.2 mm, eluting with 45% to 95% McCN/H₂O containing 0.05% NH₃) to provide 2-((6-fluoro-2-methylpyridin-3-yl)oxy)-4-methyl-N-(3-((R)—S-methyl-N—((R)-tetrahydrofuran-2-carbonyl)sulfonimidoyl)phenyl)-5-(trifluoromethyl)nicotinamide (71.4 mg, 49.5%) as a white solid. ¹H NMR (400 MHz, DMSO-d₆) δ 11.32 (s, 1H), 8.56 (s, 1H), 8.48 (s, 1H), 7.98-7.81 (m, 2H), 7.74-7.65 (m, 2H), 7.12 (dd, J=8.7, 3.3 Hz, 1H), 4.28 (dd, J=8.3, 5.1 Hz, 1H), 3.86-3.70 (m, 2H), 3.48 (s, 3H), 2.51 (s, 3H), 2.25 (s, 3H), 2.17-1.71 (m, 4H). LCMS (ESI) calcd. for C₂₆H₂₅F₄N₄O₅S [M+H]⁺ m/z 581.15, found 581.10.

### Example 15D

### 2-((6-fluoro-2-methylpyridin-3-yl)oxy)-4-methyl-N-(3-((R)—S-methyl-N—((S)-tetrahydrofuran-3-carbonyl)sulfonimidoyl)phenyl)-5-(trifluoromethyl)nicotinamide

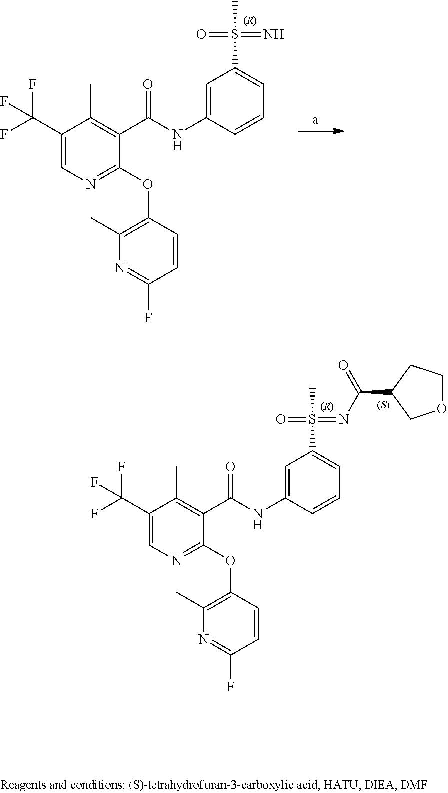

A mixture of (R)-2-((6-fluoro-2-methylpyridin-3-yl)oxy)-4-methyl-N-(3-(S-methylsulfonimidoyl)phenyl)-5-(trifluoromethyl)nicotinamide (120 mg, 0.25 mmol), (S)-tetrahydrofuran-3-carboxylic acid (57.8 mg, 0.5 mmol) and HATU (141.8 mg, 0.37 mmol) in DMF (5 mL) was added DIEA (96.4 mg, 0.75 mmol) at room temperature. The reaction mixture was stirred at room temperature for 4 hours. After the reaction was completed, the resulting solution was diluted with water (30 mL) and extracted with EtOAc (30 mL×3). The combined organic layers were washed with brine, dried over sodium sulfate, concentrated under vacuum. The residue was purified by prep-HPLC (Gemini 5 um C₁₈ column, 150*21.2 mm, eluting with 40% to 95% MeCN/H₂O containing 0.05% NH₃) to provide 2-((6-fluoro-2-methylpyridin-3-yl)oxy)-4-methyl-N-(3-((R)—S-methyl-N—((S)-tetrahydrofuran-3-carbonyl)sulfonimidoyl)phenyl)-5-(trifluoromethyl)nicotinamide (76.3 mg, 52.8%) as a white solid. ¹H NMR (400 MHz, DMSO-d₆) δ 11.31 (s, 1H), 8.56 (s, 1H), 8.48 (s, 1H), 7.95-7.80 (m, 2H), 7.74-7.66 (m, 2H), 7.11 (dd, J=8.7, 3.3 Hz, 1H), 3.87-3.74 (m, 2H), 3.71-3.57 (m, 2H), 3.46 (s, 3H), 3.10-3.00 (m, 1H), 2.51 (s, 3H), 2.25 (s, 3H), 2.10-1.93 (m, 2H). LCMS (ESI) calcd. for C₂₆H₂₅F₄N₄O₅S [M+H]⁺ m/z 581.15, found 581.15.

### Example 16D

### 2-((6-fluoro-2-methylpyridin-3-yl)oxy)-N-(3-((R)—N—((R)-2-hydroxypropanoyl)-S-methylsulfonimidoyl)phenyl)-4-methyl-5-(trifluoromethyl)nicotinamide

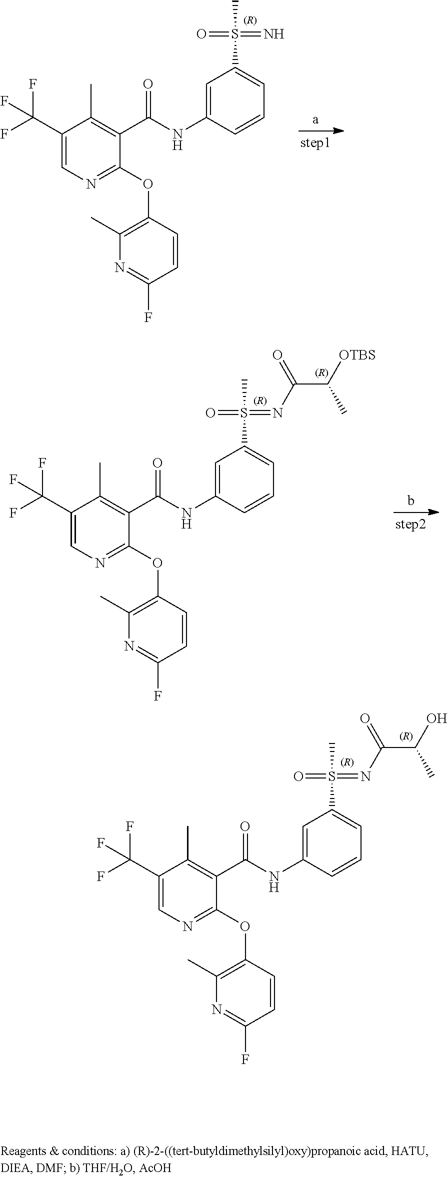

Step 1: N-(3-((R)—N—((R)-2-((tert-butyldimethylsilyl)oxy)propanoyl)-S-methylsulfonimidoyl)phenyl)-2-((6-fluoro-2-methylpyridin-3-yl)oxy)-4-methyl-5-(trifluoromethyl)nicotinamide: To a solution of (R)-2-((6-fluoro-2-methylpyridin-3-yl)oxy)-4-methyl-N-(3-(S-methylsulfonimidoyl)phenyl)-5-(trifluoromethyl)nicotinamide (150 mg, 0.31 mmol), (R)-2-((tert-butyldimethylsilyl)oxy)propanoic acid (317 mg, 1.56 mmol) and HATU (236 mg, 0.62 mmol) in DMF (5 mL) and was added DIEA (120 mg, 0.93 mmol). Then the mixture was stirred at 25° C. for 2 hours. LCMS showed the rection was completed. The mixture was diluted with water and extracted with EtOAc (3×30 mL). The combined organic layers were washed with brine, dried with Na₂SO₄, and concentrated under vacuum. The residue was purified by flash column chromatography on silica gel (PE/EtOAc=1/1) to give N-(3-((R)—N—((R)-2-((tert-butyldimethylsilyl)oxy)propanoyl)-S-methylsulfonimidoyl)phenyl)-2-((6-fluoro-2-methylpyridin-3-yl)oxy)-4-methyl-5-(trifluoromethyl)nicotinamide (100 mg, 48.12%) as a yellow oil. LCMS (ESI) calcd. for C₃₀H₃₇F₄N₄O₅SSi [M+H]⁺ m/z 669.22, found 669.20.

Step 2: 2-((6-fluoro-2-methylpyridin-3-yl)oxy)-N-(3-((R)—N—((R)-2-hydroxypropanoyl)-S-methylsulfonimidoyl)phenyl)-4-methyl-5-(trifluoromethyl)nicotinamide: To a solution of N-(3-((R)—N—((R)-2-((tert-butyldimethylsilyl)oxy)propanoyl)-S-methylsulfonimidoyl)phenyl)-2-((6-fluoro-2-methylpyridin-3-yl)oxy)-4-methyl-5-(trifluoromethyl)nicotinamide (100 mg, 0.15 mmol) in THE (1 mL) was added H₂O (3 mL) and AcOH (3 mL) at room temperature. The reaction mixture was stirred at room temperature for 2 hours. After the reaction was completed, the mixture was diluted with water and extracted with EtOAc (3×30 mL). The combined organic layers were washed with brine, dried with Na₂SO₄, and concentrated under vacuum. The residue was purified by prep-HPLC (Gemini 5 um C₁₈ column, 150*19 mm, eluting with 20% to 30% ACN/H₂O containing 0.1% formic acid) to provide 2-((6-fluoro-2-methylpyridin-3-yl)oxy)-N-(3-((R)—N—((R)-2-hydroxypropanoyl)-S-methylsulfonimidoyl)phenyl)-4-methyl-5-(trifluoromethyl)nicotinamide (55.3 mg, 64.71%) as a white solid. ¹H NMR (400 MHz, DMSO-d₆) δ 11.31 (s, 1H), 8.56 (s, 1H), 8.45 (d, J=1.7 Hz, 1H), 7.98-7.81 (m, 2H), 7.75-7.66 (m, 2H), 7.11 (dd, J=8.7, 3.4 Hz, 1H), 4.72 (d, J=5.3 Hz, 1H), 4.08-3.97 (m, 1H), 3.48 (s, 3H), 2.51 (s, 3H), 2.25 (s, 3H), 1.27 (d, J=6.8 Hz, 3H). LCMS (ESI) calcd. for C₂₄H₂₃F₄N₄O₅S [M+H]⁺ m/z 555.13, found 554.95.

### Example 17D

### tert-butyl (2S,4R)-2-(((R)-(3-(2-((6-fluoro-2-methylpyridin-3-yl)oxy)-4-methyl-5-(trifluoromethyl)nicotinamido)phenyl)(methyl)(oxo)-λ⁶-sulfaneylidene)carbamoyl)-4-hydroxypyrrolidine-1-carboxylate

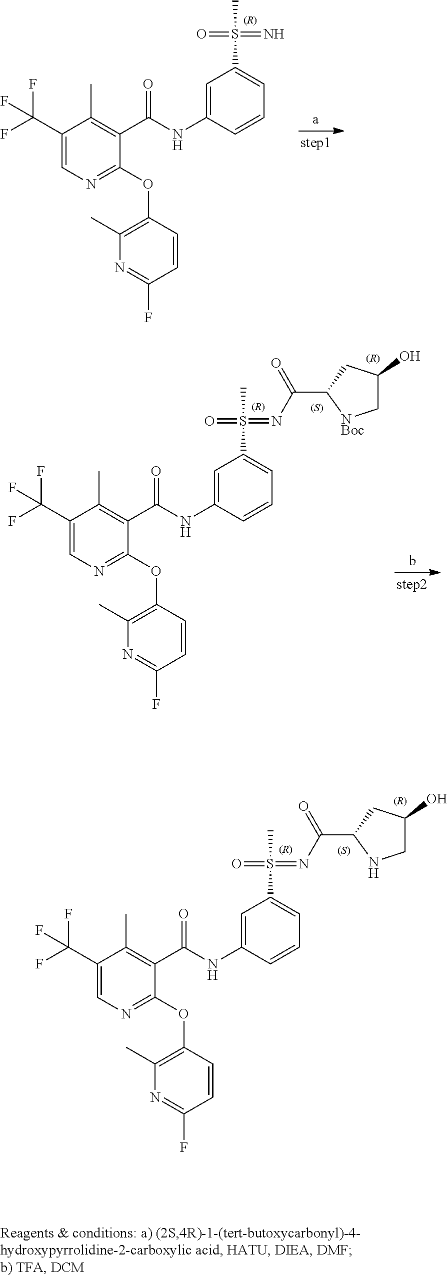

Step 1: A mixture of (R)-2-((6-fluoro-2-methylpyridin-3-yl)oxy)-4-methyl-N-(3-(S-methylsulfonimidoyl)phenyl)-5-(trifluoromethyl)nicotinamide (120 mg, 0.25 mmol), (2S,4R)-1-(tert-butoxycarbonyl)-4-hydroxypyrrolidine-2-carboxylic acid (115.5 mg, 0.50 mmol) and HATU (141.8 mg, 0.37 mmol) in DMF (5 mL) was added DIEA (96.4 mg, 0.75 mmol) at room temperature. The reaction mixture was stirred at 25° C. for 4 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to give tert-butyl (2S,4R)-2-(((R)-(3-(2-((6-fluoro-2-methylpyridin-3-yl)oxy)-4-methyl-5-(trifluoromethyl)nicotinamido)phenyl)(methyl)(oxo)-λ⁶-sulfaneylidene)carbamoyl)-4-hydroxypyrrolidine-1-carboxylate (100 mg, 57.7%) as a yellow solid. LCMS (ESI) calcd. for C₃₁H₃₄F₄N₅O₇S [M+H]⁺ m/z 696.21, found 696.10.

Step 2: A solution of tert-butyl (2S,4R)-2-(((R)-(3-(2-((6-fluoro-2-methylpyridin-3-yl)oxy)-4-methyl-5-(trifluoromethyl)nicotinamido)phenyl)(methyl)(oxo)-λ⁶-sulfaneylidene)carbamoyl)-4-hydroxypyrrolidine-1-carboxylate (100 mg, 0.14 mmol) in DCM (3 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% NH₃) to provide 2-((6-fluoro-2-methylpyridin-3-yl)oxy)-N-(3-((R)—N-((2S,4R)-4-hydroxypyrrolidine-2-carbonyl)-S-methylsulfonimidoyl)phenyl)-4-methyl-5-(trifluoromethyl)nicotinamide (42.7 mg, 47.5%) as a white solid. ¹H NMR (400 MHz, DMSO-d₆) δ 11.32 (s, 1H), 8.56 (s, 1H), 8.47 (s, 1H), 7.97-7.79 (m, 2H), 7.69 (dd, J=7.4, 5.7 Hz, 2H), 7.12 (dd, J=8.7, 3.2 Hz, 1H), 4.65 (d, J=3.3 Hz, 1H), 4.11 (s, 1H), 3.69 (t, J=7.8 Hz, 1H), 3.46 (s, 3H), 3.02 (dd, J=11, 1, 5.2 Hz, 1H), 2.58-2.52 (m, 3H), 2.25 (s, 3H), 2.02-1.59 (m, 2H). LCMS (ESI) calcd. for C₂₆H₂₆F₄N₅O₅S [M+H]⁺ m/z 596.16, found 596.10.

### Example 18D

### 3-((6-fluoro-2-methylpyridin-3-yl)oxy)-N-(3-((R)—N—((S)-2-hydroxypropanoyl)-S-methylsulfonimidoyl)phenyl)-5-methyl-6-(trifluoromethyl)pyridazine-4-carboxamide

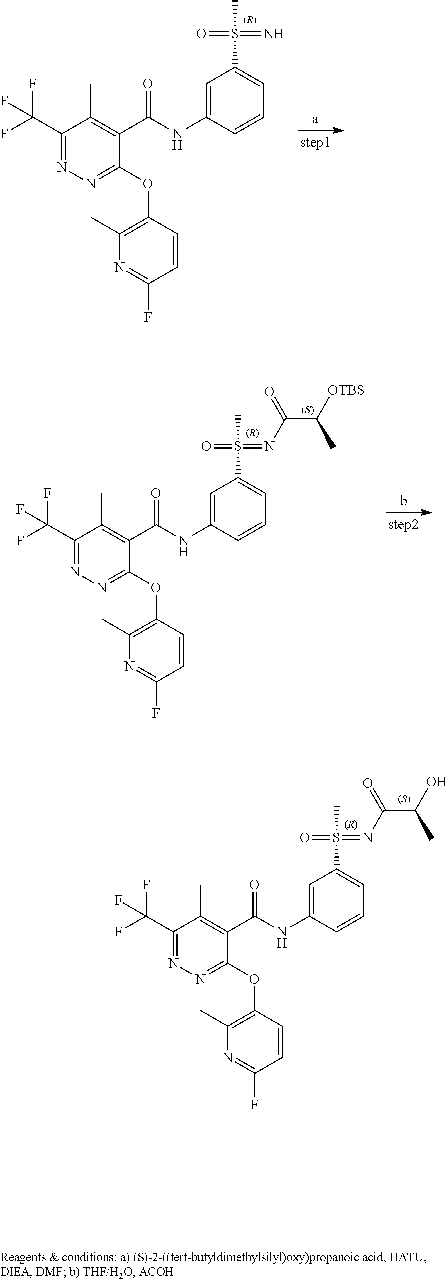

Step 1: N-(3-((R)—N—((S)-2-((tert-butyldimethylsilyl)oxy)propanoyl)-S-methylsulfonimidoyl)phenyl)-3-((6-fluoro-2-methylpyridin-3-yl)oxy)-5-methyl-6-(trifluoromethyl)pyridazine-4-carboxamide: To a mixture of (R)-3-((6-fluoro-2-methylpyridin-3-yl)oxy)-5-methyl-N-(3-(S-methylsulfonimidoyl)phenyl)-6-(trifluoromethyl)pyridazine-4-carboxamide (150 mg, 0.31 mmol), (S)-2-((tert-butyldimethylsilyl)oxy)propanoic acid (190 mg, 0.93 mmol) and HATU (235 mg, 0.62 mmol) in DMF (5 mL) was added DIEA (120 mg, 0.93 mmol) at room temperature. The reaction mixture was stirred at room temperature for 16 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to provide N-(3-((R)—N—((S)-2-((tert-butyldimethylsilyl)oxy)propanoyl)-S-methylsulfonimidoyl)phenyl)-3-((6-fluoro-2-methylpyridin-3-yl)oxy)-5-methyl-6-(trifluoromethyl)pyridazine-4-carboxamide (50 mg, 24.15%) as a yellow solid. LCMS (ESI) calcd. for C₂₉H₃₆F₄N₅O₅SSi [M+H]⁺ m/z 670.22, found 670.10.

Step 2: 3-((6-fluoro-2-methylpyridin-3-yl)oxy)-N-(3-((R)—N—((S)-2-hydroxypropanoyl)-S-methylsulfonimidoyl)phenyl)-5-methyl-6-(trifluoromethyl)pyridazine-4-carboxamide: A solution of N-(3-((R)—N—((S)-2-((tert-butyldimethylsilyl)oxy)propanoyl)-S-methylsulfonimidoyl)phenyl)-3-((6-fluoro-2-methylpyridin-3-yl)oxy)-5-methyl-6-(trifluoromethyl)pyridazine-4-carboxamide (50 mg, 0.07 mmol) in THF/H₂O/AcOH (1/3/3, 7 mL) was stirred at room temperature for 4 hours. After the reaction was completed, the resulting solution was diluted with water (30 mL) and extracted with EtOAc (30 mL×3). Then the organic solution was washed with water (20 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 90% MeCN/H₂O containing 0.1% formic acid) to provide 3-((6-fluoro-2-methylpyridin-3-yl)oxy)-N-(3-((R)—N—((S)-2-hydroxypropanoyl)-S-methylsulfonimidoyl)phenyl)-5-methyl-6-(trifluoromethyl)pyridazine-4-carboxamide (21 mg, 50.6%) as a white solid. ¹H NMR (400 MHz, DMSO-d₆) δ 11.43 (s, 1H), 8.42 (d, J=1.7 Hz, 1H), 7.98-7.87 (m, 2H), 7.77-7.69 (m, 2H), 7.16 (dd, J=8.7, 3.3 Hz, 1H), 4.68 (d, J=5.6 Hz, 1H), 4.10-3.97 (m, 1H), 3.47 (s, 3H), 2.57-2.53 (m, 3H), 2.29 (s, 3H), 1.25 (d, J=6.8 Hz, 3H). LCMS (ESI) calcd. for C₂₃H₂₂F₄N₅O₅S [M+H]⁺ m/z 556.13, found 556.00.

### Example 19

### 3-((6-fluoro-2-methylpyridin-3-yl)oxy)-N-(3-((R)—N—((R)-2-hydroxypropanoyl)-S-methylsulfonimidoyl)phenyl)-5-methyl-6-(trifluoromethyl)pyridazine-4-carboxamide

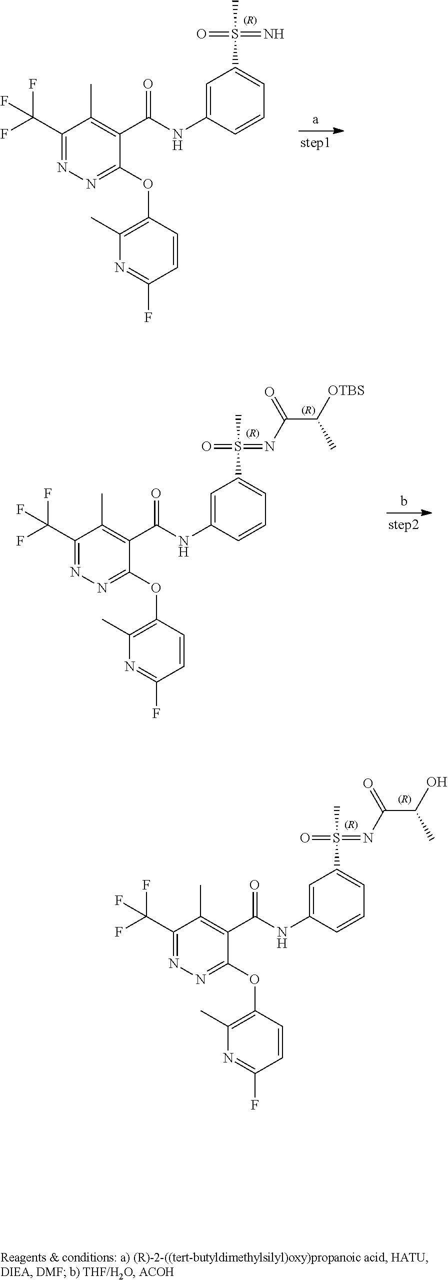

Step 1: N-(3-((R)—N—((R)-2-((tert-butyldimethylsilyl)oxy)propanoyl)-S-methylsulfonimidoyl)phenyl)-3-((6-fluoro-2-methylpyridin-3-yl)oxy)-5-methyl-6-(trifluoromethyl)pyridazine-4-carboxamide. To a mixture of (R)-3-((6-fluoro-2-methylpyridin-3-yl)oxy)-5-methyl-N-(3-(S-methylsulfonimidoyl)phenyl)-6-(trifluoromethyl)pyridazine-4-carboxamide (150 mg, 0.31 mmol), (R)-2-((tert-butyldimethylsilyl)oxy)propanoic acid (190 mg, 0.93 mmol) and HATU (235 mg, 0.62 mmol) in DMF (5 mL) was added DIEA (120 mg, 0.93 mmol) at room temperature. The reaction mixture was stirred at room temperature for 16 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to provide N-(3-((R)—N—((R)-2-((tert-butyldimethylsilyl)oxy)propanoyl)-S-methylsulfonimidoyl)phenyl)-3-((6-fluoro-2-methylpyridin-3-yl)oxy)-5-methyl-6-(trifluoromethyl)pyridazine-4-carboxamide (45 mg, 21.67% yield) as a yellow solid. LCMS (ESI) calcd. for C₂₉H₃₆F₄N₅O₅SSi [M+H]⁺ m/z 670.22, found 670.15.

Step 2: 3-((6-fluoro-2-methylpyridin-3-yl)oxy)-N-(3-((R)—N—((R)-2-hydroxypropanoyl)-S-methylsulfonimidoyl)phenyl)-5-methyl-6-(trifluoromethyl)pyridazine-4-carboxamide: A solution of N-(3-((R)—N—((R)-2-((tert-butyldimethylsilyl)oxy)propanoyl)-S-methylsulfonimidoyl)phenyl)-3-((6-fluoro-2-methylpyridin-3-yl)oxy)-5-methyl-6-(trifluoromethyl)pyridazine-4-carboxamide (45 mg, 0.07 mmol) in THF/H₂O/AcOH (1/3/3, 7 mL) was stirred at room temperature for 4 hours. After the reaction was completed, the resulting solution was diluted with water (30 mL) and extracted with EtOAc (30 mL×3). Then the organic solution was washed with water (20 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 90% MeCN/H₂O containing 0.1% formic acid) to provide 3-((6-fluoro-2-methylpyridin-3-yl)oxy)-N-(3-((R)—N—((R)-2-hydroxypropanoyl)-S-methylsulfonimidoyl)phenyl)-5-methyl-6-(trifluoromethyl)pyridazine-4-carboxamide (17 mg, 45.6%) as a white solid. ¹H NMR (400 MHz, DMSO-d₆) δ 11.42 (s, 1H), 8.41 (s, 1H), 8.00-7.87 (m, 2H), 7.78-7.67 (m, 2H), 7.16 (dd, J=8.6, 3.0 Hz, 1H), 4.70 (d, J=4.9 Hz, 1H), 4.08-3.96 (m, 1H), 3.49 (s, 3H), 2.54 (s, 3H), 2.29 (s, 3H), 1.27 (d, J=6.8 Hz, 3H). LCMS (ESI) calcd. for C₂₃H₂₂F₄N₅O₅S [M+H]⁺ m/z 556.13, found 556.05.

### Example 20D

### (R)-3-((6-fluoro-2-methylpyridin-3-yl)oxy)-N-(3-(N-(2-hydroxyacetyl)-S-methylsulfonimidoyl)phenyl)-5-methyl-6-(trifluoromethyl)pyridazine-4-carboxamide

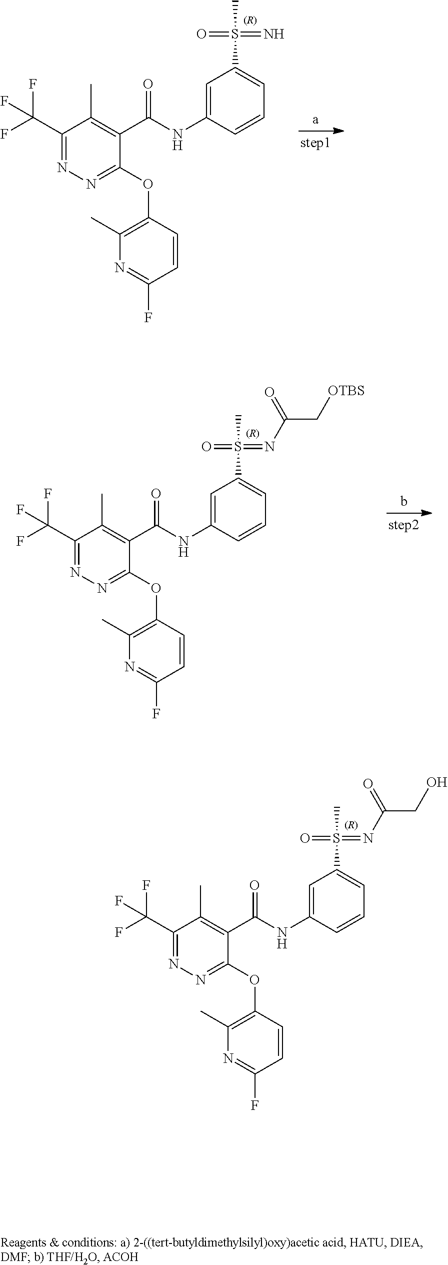

Step 1: (R)—N-(3-(N-(2-((tert-butyldimethylsilyl)oxy)acetyl)-S-methylsulfonimidoyl)phenyl)-3-((6-fluoro-2-methylpyridin-3-yl)oxy)-5-methyl-6-(trifluoromethyl)pyridazine-4-carboxamide: To a mixture of (R)-3-((6-fluoro-2-methylpyridin-3-yl)oxy)-5-methyl-N-(3-(S-methylsulfonimidoyl)phenyl)-6-(trifluoromethyl)pyridazine-4-carboxamide (150 mg, 0.31 mmol), 2-((tert-butyldimethylsilyl)oxy)acetic acid (177 mg, 0.93 mmol) and HATU (235 mg, 0.62 mmol) in DMF (5 mL) was added DIEA (120 mg, 0.93 mmol) at room temperature. The reaction mixture was stirred at room temperature for 16 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to provide (R)—N-(3-(N-(2-((tert-butyldimethylsilyl)oxy)acetyl)-S-methylsulfonimidoyl)phenyl)-3-((6-fluoro-2-methylpyridin-3-yl)oxy)-5-methyl-6-(trifluoromethyl)pyridazine-4-carboxamide (55 mg, 27.09%) as a yellow solid. LCMS (ESI) calcd. for C₂₈H34F₄N5O₅SSi [M+H]⁺ m/z 656.20, found 656.05.

Step 2: (R)-3-((6-fluoro-2-methylpyridin-3-yl)oxy)-N-(3-(N-(2-hydroxyacetyl)-S-methylsulfonimidoyl)phenyl)-5-methyl-6-(trifluoromethyl)pyridazine-4-carboxamide: A solution of (R)—N-(3-(N-(2-((tert-butyldimethylsilyl)oxy)acetyl)-S-methylsulfonimidoyl)phenyl)-3-((6-fluoro-2-methylpyridin-3-yl)oxy)-5-methyl-6-(trifluoromethyl)pyridazine-4-carboxamide (55 mg, 0.08 mmol) in THF/H₂O/AcOH (1/3/3, 7 mL) was stirred at room temperature for 4 hours. After the reaction was completed, the resulting solution was diluted with water (30 mL) and extracted with EtOAc (30 mL×3). Then the organic solution was washed with water (20 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 90% MeCN/H₂O containing 0.1% formic acid) to provide (R)-3-((6-fluoro-2-methylpyridin-3-yl)oxy)-N-(3-(N-(2-hydroxyacetyl)-S-methylsulfonimidoyl)phenyl)-5-methyl-6-(trifluoromethyl)pyridazine-4-carboxamide (16 mg, 35.56%) as a white solid. ¹H NMR (400 MHz, DMSO-d₆) δ 11.46 (s, 1H), 8.38 (d, J=1.8 Hz, 1H), 8.00-7.90 (m, 2H), 7.79-7.67 (m, 2H), 7.17 (dd, J=8.7, 3.2 Hz, 1H), 4.80 (t, J=6.3 Hz, 1H), 3.95 (d, J=6.3 Hz, 2H), 3.48 (s, 3H), 2.55 (d, J=1.3 Hz, 3H), 2.29 (s, 3H). LCMS (ESI) calcd. for C₂₂H₂₀F₄N₅O₅S [M+H]⁺ m/z 542.11, found 542.00.

### Example 21D

### N-(3-((R)—N-(D-alanyl)-S-methylsulfonimidoyl)phenyl)-3-((6-fluoro-2-methylpyridin-3-yl)oxy)-5-methyl-6-(trifluoromethyl)pyridazine-4-carboxamide

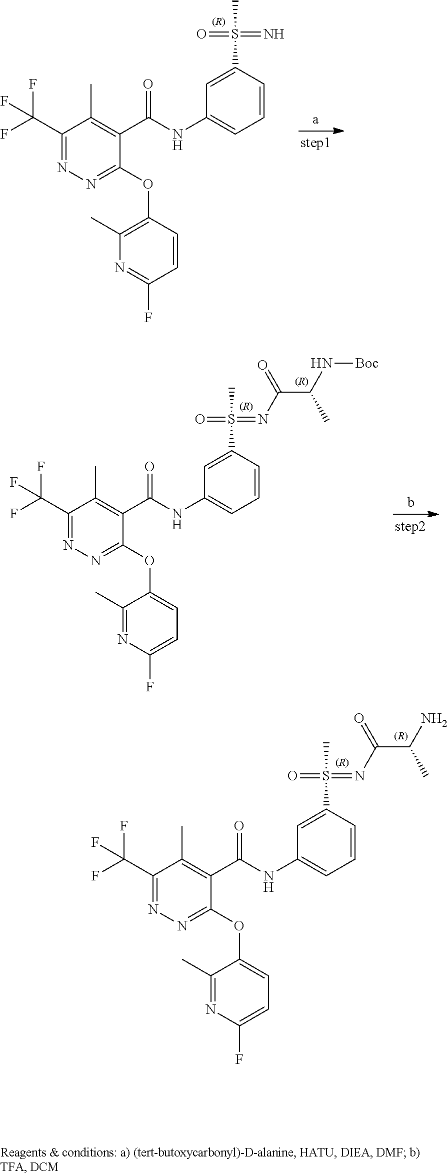

Step 1: tert-butyl ((R)-1-(((R)-(3-(3-((6-fluoro-2-methylpyridin-3-yl)oxy)-5-methyl-6-(trifluoromethyl)pyridazine-4-carboxamido)phenyl)(methyl)(oxo)-λ⁶-sulfaneylidene)amino)-1-oxopropan-2-yl)carbamate. A mixture of (R)-3-((6-fluoro-2-methylpyridin-3-yl)oxy)-5-methyl-N-(3-(S-methylsulfonimidoyl)phenyl)-6-(trifluoromethyl)pyridazine-4-carboxamide (150 mg, 0.31 mmol) and (tert-butoxycarbonyl)-D-alanine (88 mg, 0.46 mmol) in DMF (5 mL) was added HATU (177 mg, 0.47 mmol) and DIEA (120 mg, 0.93 mmol). The mixture was stirred at room temperature for 4 hours. The reaction was monitored by L CMS. After the reaction was completed, the resulting solution was diluted with water (40 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give tert-butyl ((R)-1-(((R)-(3-(3-((6-fluoro-2-methylpyridin-3-yl)oxy)-5-methyl-6-(trifluoromethyl)pyridazine-4-carboxamido)phenyl)(methyl)(oxo)-λ⁶-sulfaneylidene)amino)-1-oxopropan-2-yl)carbamate (120 mg, 59.07%) as a white oil. LCMS (ESI) calcd. for C₂₈H₃₀F₄N₆O₆SNa [M+H]⁺ m/z 677.18, found 677.15.

Step 2: N-(3-((R)—N-(D-alanyl)-S-methylsulfonimidoyl)phenyl)-3-((6-fluoro-2-methylpyridin-3-yl)oxy)-5-methyl-6-(trifluoromethyl)pyridazine-4-carboxamide: A solution of tert-butyl ((R)-1-(((R)-(3-(3-((6-fluoro-2-methylpyridin-3-yl)oxy)-5-methyl-6-(trifluoromethyl)pyridazine-4-carboxamido)phenyl)(methyl)(oxo)-λ⁶-sulfaneylidene)amino)-1-oxopropan-2-yl)carbamate (120 mg, 0.18 mmol) in DCM (10 mL) and TFA (1 mL) at room temperature. The reaction mixture was stirred at room temperature for 2 hours. After the reaction was completed, the solution was diluted with water (30 mL) and extracted with DCM (20 mL×3). The combined organic phases were washed with brine, dried over sodium sulfate, and concentrated under vacuum. The residue was purified by prep-HPLC (Gemini 5 um C₁₈ column, 150*21.2 mm, eluting with 35% to 45% to 85% McCN/120 containing 0.05% NH₃) to provide N-(3-((R)—N-(D-alanyl)-S-methylsulfonimidoyl)phenyl)-3-((6-fluoro-2-methylpyridin-3-yl)oxy)-5-methyl-6-(trifluoromethyl)pyridazine-4-carboxamide (18.2 mg, 16.89%) as a white solid. ¹H NMR (400 MHz, DMSO-d₆) δ 11.44 (bs, 1H), 8.41 (d, J=1.7 Hz, 1H), 8.00-7.88 (m, 2H), 7.77-7.68 (m, 2H), 7.17 (dd, J=8.7, 3.3 Hz, 1H), 3.47 (s, 3H), 3.30 (s, 1H), 2.54 (d, J=1.3 Hz, 3H), 2.29 (s, 3H), 1.85 (bs, 2H), 1.17 (t, J=6.3 Hz, 3H). LCMS (ESI) calcd. for C₂₃H₂₃F₄N₆O₄S [M+H]⁺ m/z 555.15, found 555.15.

### Example 22D

### N-(3-((R)—N-(L-prolyl)-S-methylsulfonimidoyl)phenyl)-2-((6-fluoro-2-methylpyridin-3-yl)oxy)-4-methyl-5-(trifluoromethyl)nicotinamide

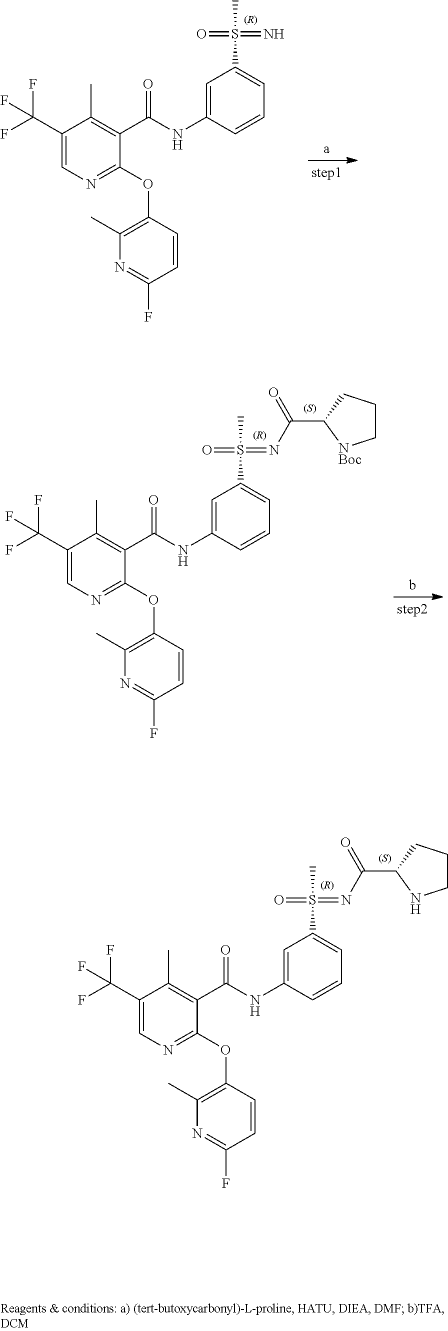

Step 1: tert-butyl (S)-2-(((R)-(3-(2-((6-fluoro-2-methylpyridin-3-yl)oxy)-4-methyl-5-(trifluoromethyl)nicotinamido)phenyl)(methyl)(oxo)-λ⁶-sulfaneylidene)carbamoyl)pyrrolidine-1-carboxylate: A mixture of (R)-2-((6-fluoro-2-methylpyridin-3-yl)oxy)-4-methyl-N-(3-(S-methylsulfonimidoyl)phenyl)-5-(trifluoromethyl)nicotinamide (150 mg, 0.31 mmol), (tert-butoxycarbonyl)-L-proline (134.5 mg, 0.62 mmol) and HATU (177.3 mg, 0.47 mmol) in DMF (5 mL) was added DIEA (120.5 mg, 0.93 mmol) at room temperature. The reaction mixture was heated at 25° C. for 4 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to give tert-butyl (S)-2-(((R)-(3-(2-((6-fluoro-2-methylpyridin-3-yl)oxy)-4-methyl-5-(trifluoromethyl)nicotinamido)phenyl)(methyl)(oxo)-λ⁶-sulfaneylidene)carbamoyl)pyrrolidine-1-carboxylate (120 mg, 56.8%) as a yellow solid. LCMS (ESI) calcd. for C₃₁H₃₄F₄N₅O₆S [M+H]⁺ m/z 680.22, found 680.10.

Step 2: N-(3-((R)—N-(L-prolyl)-S-methylsulfonimidoyl)phenyl)-2-((6-fluoro-2-methylpyridin-3-yl)oxy)-4-methyl-5-(trifluoromethyl)nicotinamide: A solution of tert-butyl (S)-2-(((R)-(3-(2-((6-fluoro-2-methylpyridin-3-yl)oxy)-4-methyl-5-(trifluoromethyl)nicotinamido)phenyl)(methyl)(oxo)-λ⁶-sulfaneylidene)carbamoyl)pyrrolidine-1-carboxylate (120 mg, 0.18 mmol) in DCM (3 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% NH₃) to provide N-(3-((R)—N-(L-prolyl)-S-methylsulfonimidoyl)phenyl)-2-((6-fluoro-2-methylpyridin-3-yl)oxy)-4-methyl-5-(trifluoromethyl)nicotinamide (62.30 mg, 59.9%) as a white solid. ¹H NMR (400 MHz, DMSO-d₆) δ 11.31 (s, 1H), 8.56 (s, 1H), 8.49 (s, 1H), 7.95-7.80 (m, 2H), 7.75-7.66 (m, 2H), 7.16-7.07 (m, 1H), 3.57-3.49 (m, 1H), 3.46 (s, 3H), 2.93-2.81 (m, 1H), 2.71-2.57 (m, 1H), 2.51 (s, 3H), 2.24 (s, 3H), 2.04-1.91 (m, 1H), 1.83-1.70 (m, 1H), 1.64-1.42 (m, 2H). LCMS (ESI) calcd. for C₂₆H₂₆F₄N₅O₄S [M+H]⁺ m/z 580.17, found 580.10.

### Example 23D

### N-(3-((R)—N-(D-prolyl)-S-methylsulfonimidoyl)phenyl)-2-((6-fluoro-2-methylpyridin-3-yl)oxy)-4-methyl-5-(trifluoromethyl)nicotinamide formate

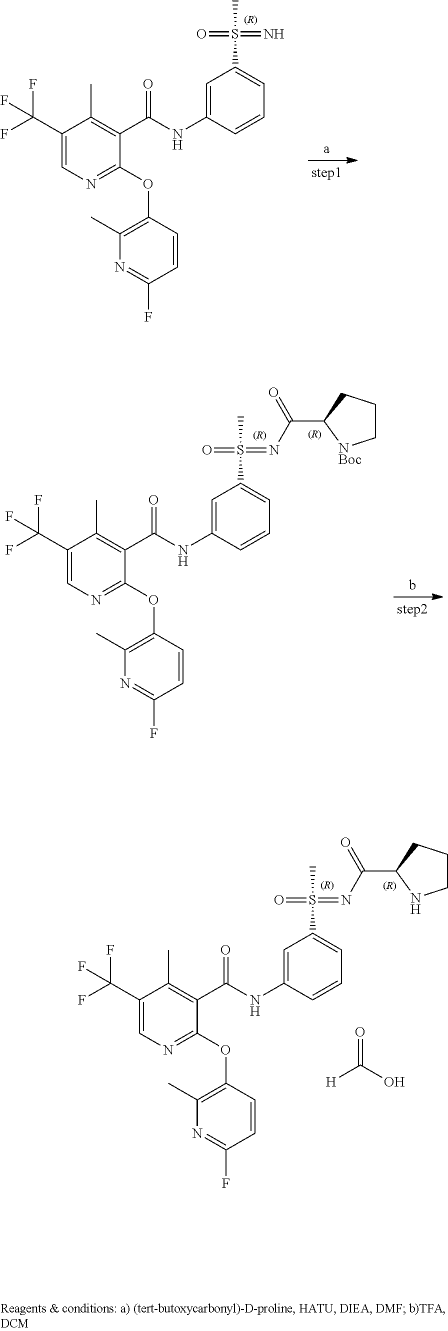

Step 1: tert-butyl (R)-2-(((R)-(3-(2-((6-fluoro-2-methylpyridin-3-yl)oxy)-4-methyl-5-(trifluoromethyl)nicotinamido)phenyl)(methyl)(oxo)-λ⁶-sulfaneylidene)carbamoyl)pyrrolidine-1-carboxylate: A mixture of (R)-2-((6-fluoro-2-methylpyridin-3-yl)oxy)-4-methyl-N-(3-(S-methylsulfonimidoyl)phenyl)-5-(trifluoromethyl)nicotinamide 150 mg, 0.31 mmol), (tert-butoxycarbonyl)-D-proline (134.5 mg, 0.62 mmol) and HATU (177.3 mg, 0.47 mmol) in DMF (5 mL) was added DIEA (120.5 mg, 0.93 mmol) at room temperature. The reaction mixture was stirred at 25° C. for 4 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to tert-butyl (R)-2-(((R)-(3-(2-((6-fluoro-2-methylpyridin-3-yl)oxy)-4-methyl-5-(trifluoromethyl)nicotinamido)phenyl)(methyl)(oxo)-λ⁶-sulfaneylidene)carbamoyl)pyrrolidine-1-carboxylate (120 mg, 56.8%) as a yellow solid. LCMS (ESI) calcd. for C₃₁H₃₄F₄N₅O₆S [M+H]⁺ m/z 680.22, found 680.15.

Step 2: N-(3-((R)—N-(D-prolyl)-S-methylsulfonimidoyl)phenyl)-2-((6-fluoro-2-methylpyridin-3-yl)oxy)-4-methyl-5-(trifluoromethyl)nicotinamide formate: A solution of tert-butyl (R)-2-(((R)-(3-(2-((6-fluoro-2-methylpyridin-3-yl)oxy)-4-methyl-5-(trifluoromethyl) nicotinamido)phenyl)(methyl)(oxo)-λ⁶-sulfaneylidene)carbamoyl)pyrrolidine-1-carboxylate (120 mg, 0.18 mmol) in DCM (3 mL) was added TFA (0.5 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (20 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 95% MeCN/H₂O containing 0.05% formic acid) to provide N-(3-((R)—N-(D-prolyl)-S-methylsulfonimidoyl)phenyl)-2-((6-fluoro-2-methylpyridin-3-yl)oxy)-4-methyl-5-(trifluoromethyl)nicotinamide formate (61.20 mg, 59.8%) as a white solid. ¹H NMR (400 MHz, DMSO-d₆) δ 11.43 (s, 1H), 8.56 (s, 1H), 8.49 (s, 1H), 8.24 (s, 1H), 7.95 (d, J=7.7 Hz, 1H), 7.89-7.81 (m, 1H), 7.77-7.67 (m, 2H), 7.12 (dd, J=8.6, 3.3 Hz, 1H), 3.88-3.80 (m, 1H), 3.53 (s, 3H), 3.08-2.99 (m, 1H), 2.86 (s, 1H), 2.51 (s, 3H), 2.25 (s, 3H), 2.14-1.84 (m, 2H), 1.80-1.62 (m, 2H). LCMS (ESI) calcd. for C₂₆H₂₆F₄N₅O₄S [M+H]⁺ m/z 580.17, found 580.10.

### Example 24D

### (R)-3-((6-fluoro-2-methylpyridin-3-yl)oxy)-N-(3-(N-glycyl-S-methylsulfonimidoyl)phenyl)-5-methyl-6-(trifluoromethyl)pyridazine-4-carboxamide

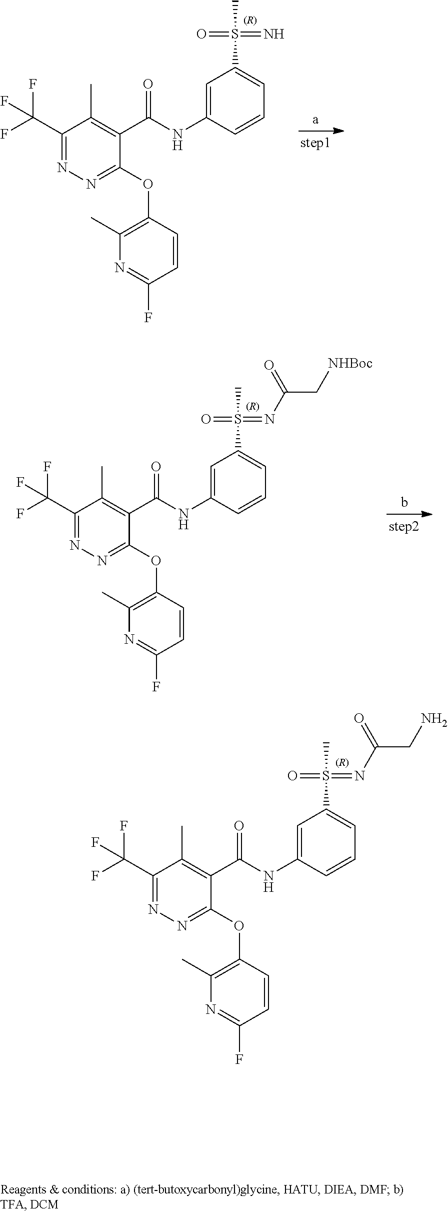

Step 1: tert-butyl (R)-(2-(((3-(3-((6-fluoro-2-methylpyridin-3-yl)oxy)-5-methyl-6-(trifluoromethyl)pyridazine-4-carboxamido)phenyl)(methyl)(oxo)-λ⁶-sulfaneylidene)amino)-2-oxoethyl)carbamate: To a solution of (R)-3-((6-fluoro-2-methylpyridin-3-yl)oxy)-5-methyl-N-(3-(S-methylsulfonimidoyl)phenyl)-6-(trifluoromethyl)pyridazine-4-carboxamide (120 mg, 0.25 mmol), (tert-butoxycarbonyl)glycine (88 mg, 0.50 mmol) and HATU (144 mg, 0.38 mmol) in DMF (3 mL) and was added DIEA (97 mg, 0.75 mmol). Then the mixture was stirred at 25° C. for 3 hours. LCMS showed the rection was completed. The mixture was diluted with water and extracted with EtOAc (3×6 mL). The combined organic layers were washed with brine, dried with Na₂SO₄, and concentrated under vacuum. The residue was purified by silica gel column chromatography (eluting with MeOH/DCM, 0 to 10%) to give tert-butyl (R)-(2-(((3-(3-((6-fluoro-2-methylpyridin-3-yl)oxy)-5-methyl-6-(trifluoromethyl)pyridazine-4-carboxamido)phenyl)(methyl)(oxo)-λ⁶-sulfaneylidene)amino)-2-oxoethyl)carbamate (110 mg, 68.75%) as a yellow oil. LCMS (ESI) calcd. for C₂₇H₂₈F₄N₆O₆SNa [M+Na]⁺ m/z 663.17, found 663.10.

Step 2: (R)-3-((6-fluoro-2-methylpyridin-3-yl)oxy)-N-(3-(N-glycyl-S-methylsulfonimidoyl)phenyl)-5-methyl-6-(trifluoromethyl)pyridazine-4-carboxamide: A solution of tert-butyl (R)-(2-(((3-(3-((6-fluoro-2-methylpyridin-3-yl)oxy)-5-methyl-6-(trifluoromethyl)pyridazine-4-carboxamido)phenyl)(methyl)(oxo)-λ⁶-sulfaneylidene)amino)-2-oxoethyl)carbamate (110 mg, 0.17 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (10 mL×3). The combined organic layers were washed with brine, dried over sodium sulfate, concentrated under vacuum. The residue was purified by prep-HPLC (Gemini 5 um C₁₈ column, 150*19 mm, eluting with 35% to 85% ACN/H₂O containing 0.05% NH₃) to provide (R)-3-((6-fluoro-2-methylpyridin-3-yl)oxy)-N-(3-(N-glycyl-S-methylsulfonimidoyl)phenyl)-5-methyl-6-(trifluoromethyl)pyridazine-4-carboxamide (18.10 mg, 19.72%) as a white solid. LCMS (ESI) calcd. for C₂₂H₂₁F₄N₆O₄S [M+H]⁺ m/z 541.12, found 541.10. ¹H NMR (400 MHz, CD₃OD) δ 8.50 (t, J=1.9 Hz, 1H), 7.96 (dd, J=8.1, 1.1 Hz, 1H), 7.87-7.81 (m, 2H), 7.70 (t, J=8.0 Hz, 1H), 7.01 (dd, J=8.6, 3.2 Hz, 1H), 3.45 (s, 3H), 3.43 (s, 2H), 2.60 (d, J=1.4 Hz, 3H), 2.33 (s, 3H).

### Example 25D

### (R)-3-((6-fluoro-2-methylpyridin-3-yl)oxy)-5-methyl-N-(3-(S-methyl-N-(methylglycyl)sulfonimidoyl)phenyl)-6-(trifluoromethyl)pyridazine-4-carboxamide

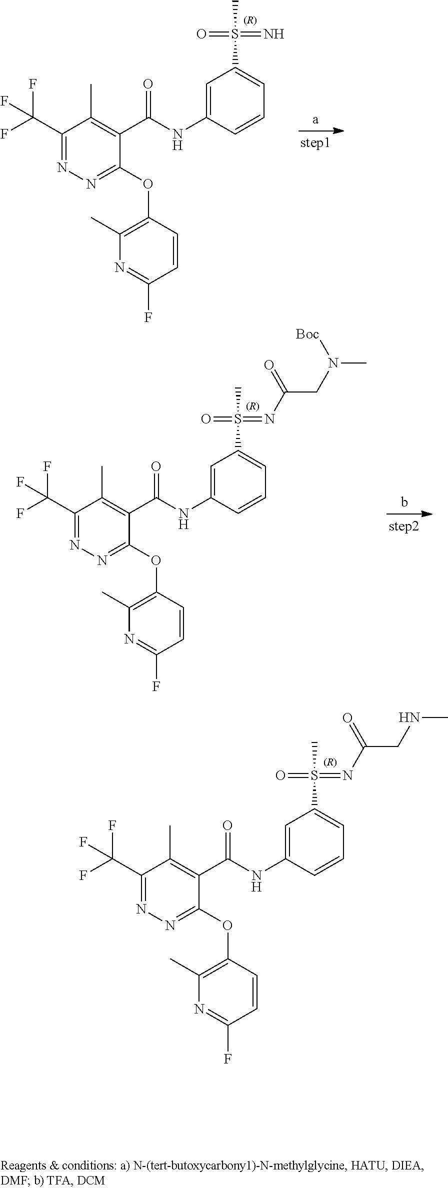

Step 1: tert-butyl (R)-(2-(((3-(3-((6-fluoro-2-methylpyridin-3-yl)oxy)-5-methyl-6-(trifluoromethyl)pyridazine-4-carboxamido)phenyl)(methyl)(oxo)-λ⁶-sulfaneylidene)amino)-2-oxoethyl)(methyl)carbamate: To a solution of (R)-3-((6-fluoro-2-methylpyridin-3-yl)oxy)-5-methyl-N-(3-(S-methylsulfonimidoyl)phenyl)-6-(trifluoromethyl)pyridazine-4-carboxamide (120 mg, 0.25 mmol), N-(tert-butoxycarbonyl)-N-methylglycine (95 mg, 0.50 mmol) and HATU (144 mg, 0.38 mmol) in DMF (3 mL) and was added DIEA (97 mg, 0.75 mmol). Then the mixture was stirred at 25° C. for 3 hours. LCMS showed the rection was completed. The mixture was diluted with water and extracted with EtOAc (3×6 mL). The combined organic phases were washed with brine, dried with Na₂SO₄, and concentrated under vacuum. The residue was purified by silica gel column chromatography (eluting with MeOH/DCM, 0 to 10%) to tert-butyl (R)-(2-(((3-(3-((6-fluoro-2-methylpyridin-3-yl)oxy)-5-methyl-6-(trifluoromethyl)pyridazine-4-carboxamido)phenyl)(methyl)(oxo)-λ⁶-sulfaneylidene)amino)-2-oxoethyl)(methyl)carbamate (112 mg, 68.50%) as a yellow oil. LCMS (ESI) calcd. for C₂₈H₃₀F₄N₆O₆SNa [M+Na]⁺ m/z 677.19, found 677.10.

Step 2: (R)-3-((6-fluoro-2-methylpyridin-3-yl)oxy)-5-methyl-N-(3-(S-methyl-N-(methylglycyl)sulfonimidoyl)phenyl)-6-(trifluoromethyl)pyridazine-4-carboxamide A solution of tert-butyl (R)-(2-(((3-(3-((6-fluoro-2-methylpyridin-3-yl)oxy)-5-methyl-6-(trifluoromethyl)pyridazine-4-carboxamido)phenyl)(methyl)(oxo)-λ⁶-sulfaneylidene)amino)-2-oxoethyl)(methyl)carbamate (110 mg, 0.17 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (10 mL×3). The combined organic layers were washed with brine, dried over sodium sulfate, concentrated under vacuum. The residue was purified by prep-HPLC (Gemini 5 um C₁₈ column, 150*19 mm, eluting with 30% to 80% ACN/H₂O containing 0.05% NH₃) to provide (R)-3-((6-fluoro-2-methylpyridin-3-yl)oxy)-5-methyl-N-(3-(S-methyl-N-(methylglycyl)sulfonimidoyl)phenyl)-6-(trifluoromethyl)pyridazine-4-carboxamide (21.20 mg, 22.51%) as a white solid. ¹H NMR (400 MHz, CD₃OD) δ 8.51 (t, J=1.9 Hz, 1H), 7.95 (ddd, J=8.1, 2.1, 0.9 Hz, 1H), 7.86-7.81 (m, 2H), 7.70 (t, J=8.0 Hz, 1H), 7.01 (dd, J=8.5, 3.1 Hz, 1H), 3.45 (s, 3H), 3.37 (s, 2H), 2.60 (d, J=1.4 Hz, 3H), 2.37 (s, 3H), 2.33 (s, 3H). LCMS (ESI) calcd. for C₂₃H₂₃F₄N₆O₄S [M+H]⁺ m/z 555.14, found 555.10.

### Example 26D

### N-(3-((R)—N-(L-prolyl)-S-methylsulfonimidoyl)phenyl)-3-((6-fluoro-2-methylpyridin-3-yl)oxy)-5-methyl-6-(trifluoromethyl)pyridazine-4-carboxamide

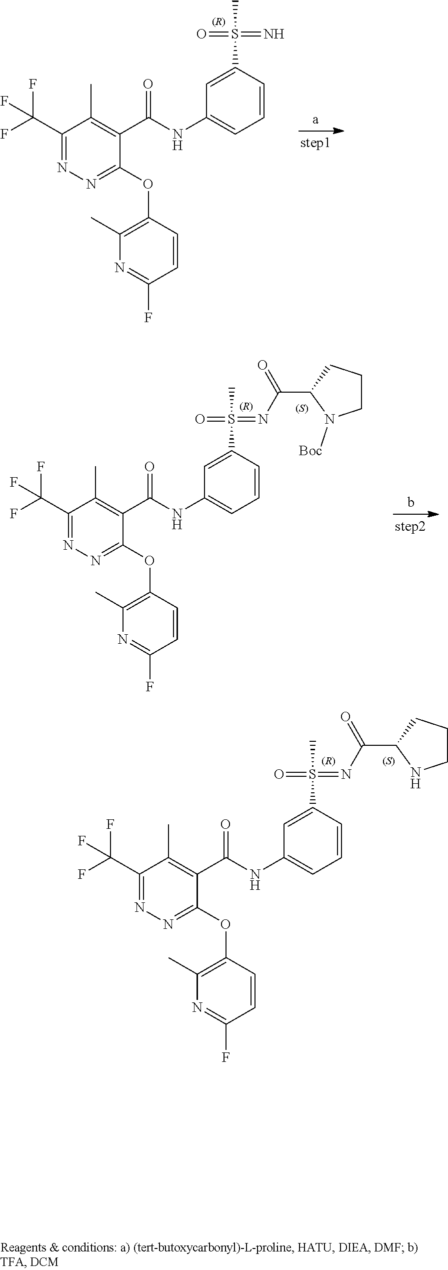

Step 1: tert-butyl (S)-2-(((R)-(3-(3-((6-fluoro-2-methylpyridin-3-yl)oxy)-5-methyl-6-(trifluoromethyl)pyridazine-4-carboxamido)phenyl)(methyl)(oxo)-λ⁶-sulfaneylidene)carbamoyl)pyrrolidine-1l-carboxylate. A mixture of (R)-3-((6-fluoro-2-methylpyridin-3-yl)oxy)-5-methyl-N-(3-(S-methylsulfonimidoyl)phenyl)-6-(trifluoromethyl)pyridazine-4-carboxamide (150 mg, 0.31 mmol) and (tert-butoxycarbonyl)-L-proline (100.66 mg, 0.465 mmol) in DMF (5 mL) was added HATU (176.98 mg, 0.465 mmol) and DIEA (120.31 mg, 0.93 mmol). The mixture was stirred at room temperature for 16 hours. The reaction was monitored by LCMS. After the reaction was completed, the resulting solution was diluted with water (50 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give tert-butyl (S)-2-(((R)-(3-(3-((6-fluoro-2-methylpyridin-3-yl)oxy)-5-methyl-6-(trifluoromethyl)pyridazine-4-carboxamido)phenyl)(methyl)(oxo)-λ⁶-sulfaneylidene)carbamoyl)pyrrolidine-1l-carboxylate (120 mg, 51.05%) as a yellow solid. LCMS (ESI) calcd. for C₃₀H₃₃F₄N₆O₆S [M+H]⁺ m/z 681.21, found 681.10.

Step 2: N-(3-((R)—N-(L-prolyl)-S-methylsulfonimidoyl)phenyl)-3-((6-fluoro-2-methylpyridin-3-yl)oxy)-5-methyl-6-(trifluoromethyl)pyridazine-4-carboxamide A solution of tert-butyl (S)-2-(((R)-(3-(3-((6-fluoro-2-methylpyridin-3-yl)oxy)-5-methyl-6-(trifluoromethyl)pyridazine-4-carboxamido)phenyl)(methyl)(oxo)-λ⁶-sulfaneylidene)carbamoyl)pyrrolidine-1-carboxylate (120 mg, 0.176 mmol) in DCM (10 mL) was added TFA (1 mL) at room temperature. The reaction mixture was stirred at room temperature for 2 hours. The residue was adjusted to pH=8-9 with saturated aqueous NaHC₃. Then the aqueous solution was extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 50% to 95% MeCN/H₂O containing 0.05% NH₃) to obtain N-(3-((R)—N-(L-prolyl)-S-methylsulfonimidoyl)phenyl)-3-((6-fluoro-2-methylpyridin-3-yl)oxy)-5-methyl-6-(trifluoromethyl)pyridazine-4-carboxamide (31.9 mg, 29.94%) as a white solid. ¹H NMR (400 MHz, DMSO-d₆) δ 11.50 (bs, 1H), 8.45 (s, 1H), 7.99-7.82 (m, 2H), 7.76-7.68 (m, 2H), 7.17 (dd, J=8.7, 3.3 Hz, 1H), 3.52 (dd, J=8.5, 5.8 Hz, 1H), 3.47 (s, 3H), 2.93-2.82 (m, 1H), 2.70-2.57 (m, 1H), 2.54 (s, 3H), 2.27 (s, 3H), 2.05-1.95 (m, 1H), 1.81-1.71 (m, 1H), 1.68-1.41 (m, 2H). LCMS (ESI) calcd. C₂₅H₂₅F₄N₆O₄S [M+H]⁺ m/z 581.16, found 580.90.

### Example 27D

### 2-((6-fluoro-2-methylpyridin-3-yl)oxy)-N-(3-((R)—N-((2S,4S)-4-hydroxypyrrolidine-2-carbonyl)-S-methylsulfonimidoyl)phenyl)-4-methyl-5-(trifluoromethyl)nicotinamide

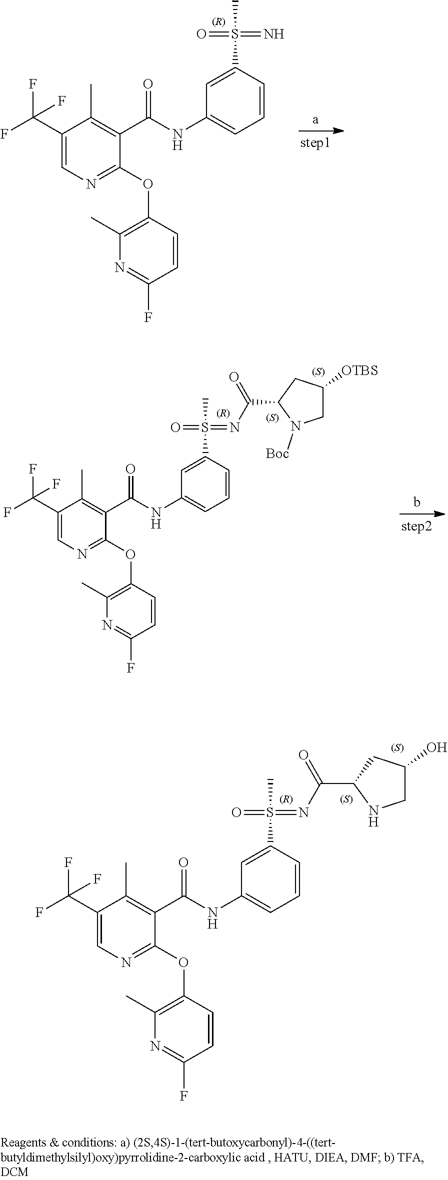

Step 1: tert-butyl (2S,4S)-4-((tert-butyldimethylsilyl)oxy)-2-(((R)-(3-(2-((6-fluoro-2-methylpyridin-3-yl)oxy)-4-methyl-5-(trifluoromethyl)nicotinamido)phenyl)(methyl)(oxo)-λ⁶-sulfaneylidene)carbamoyl)pyrrolidine-1-carboxylate: A mixture of (R)-2-((6-fluoro-2-methylpyridin-3-yl)oxy)-4-methyl-N-(3-(S-methylsulfonimidoyl)phenyl)-5-(trifluoromethyl)nicotinamide (150 mg, 0.31 mmol) and (2S,4S)-1-(tert-butoxycarbonyl)-4-((tert-butyldimethylsilyl)oxy)pyrrolidine-2-carboxylic acid (321 mg, 0.93 mmol) in DMF (5 mL) was added HATU (236 mg, 0.62 mmol) and DIEA (120 mg, 0.93 mmol). The mixture was stirred at room temperature for 16 hours. The reaction was monitored by LCMS. After the reaction was completed, the resulting solution was diluted with water (50 mL) and extracted with DCM (20 mL×3). The combined organic phases were washed with brine, dried over Na₂SO₄, concentrated under vacuum. The residue was purified by flash column chromatography on silica gel (PE/EtOAc=2/1 to 1/1) to give tert-butyl (2S,4S)-4-((tert-butyldimethylsilyl)oxy)-2-(((R)-(3-(2-((6-fluoro-2-methylpyridin-3-yl)oxy)-4-methyl-5-(trifluoromethyl)nicotinamido)phenyl)(methyl)(oxo)-λ⁶-sulfaneylidene)carbamoyl)pyrrolidine-1-carboxylate (90 mg, 35.9%) as a yellow solid. LCMS (ESI) calcd. for C₃₇H₄₈F₄N₅O₇SSi [M+H]⁺ m/z 810.30, found 810.15.

Step 2: 2-((6-fluoro-2-methylpyridin-3-yl)oxy)-N-(3-((R)—N-((2S,4S)-4-hydroxypyrrolidine-2-carbonyl)-S-methylsulfonimidoyl)phenyl)-4-methyl-5-(trifluoromethyl)nicotinamide: A mixture of tert-butyl (2S,4S)-4-((tert-butyldimethylsilyl)oxy)-2-(((R)-(3-(2-((6-fluoro-2-methylpyridin-3-yl)oxy)-4-methyl-5-(trifluoromethyl)nicotinamido)phenyl)(methyl)(oxo)-λ⁶-sulfaneylidene)carbamoyl)pyrrolidine-1-carboxylate (90 mg, 0.11 mmol) in DCM (3 mL) was added TFA (0.3 mL) at room temperature. The reaction mixture was stirred at room temperature for 2 hours. After the reaction was completed, the mixture was concentrated. The residue was adjusted to pH=8-9 with saturated aqueous NaHCO₃. Then the aqueous solution was extracted with DCM (10 mL×3). The combined organic phases were washed with brine, dried over sodium sulfate, and concentrated under vacuum. The residue was purified by prep-HPLC (Gemini 5 um C₁₈ column, 150*21.2 mm, eluting with 40% to 90% MeCN/H₂O containing 0.05% NH₃) to obtain 2-((6-fluoro-2-methylpyridin-3-yl)oxy)-N-(3-((R)—N-((2S,4S)-4-hydroxypyrrolidine-2-carbonyl)-S-methylsulfonimidoyl)phenyl)-4-methyl-5-(trifluoromethyl)nicotinamide (15.40 mg, 23.3%) as a white solid. ¹H NMR (400 MHz, DMSO-d₆) δ 11.05 (s, 1H), 8.49 (s, 1H), 8.39 (s, 1H), 7.97 (d, J=8.2 Hz, 1H), 7.82-7.58 (m, 3H), 7.04 (dd, J=8.6, 3.4 Hz, 1H), 4.21 (d, J=3.8 Hz, 1H), 4.11 (s, 1H), 3.51 (dd, J=9.2, 6.0 Hz, 1H), 3.44 (s, 3H), 2.83-2.66 (m, 2H), 2.51 (d, J=1.1 Hz, 3H), 2.25 (s, 3H), 2.21-2.11 (m, 1H), 1.78-1.65 (m, 1H). LCMS (ESI) calcd. for C₂₆H₂₆F₄N₅O₅S [M+H]⁺ m/z 596.16, found 596.10.

### Example 28D

### (R)—N-(3-(N-(2-(3,3-difluoroazetidin-1-yl)acetyl)-S-methylsulfonimidoyl)phenyl)-3-((6-fluoro-2-methylpyridin-3-yl)oxy)-5-methyl-6-(trifluoromethyl)pyridazine-4-carboxamide

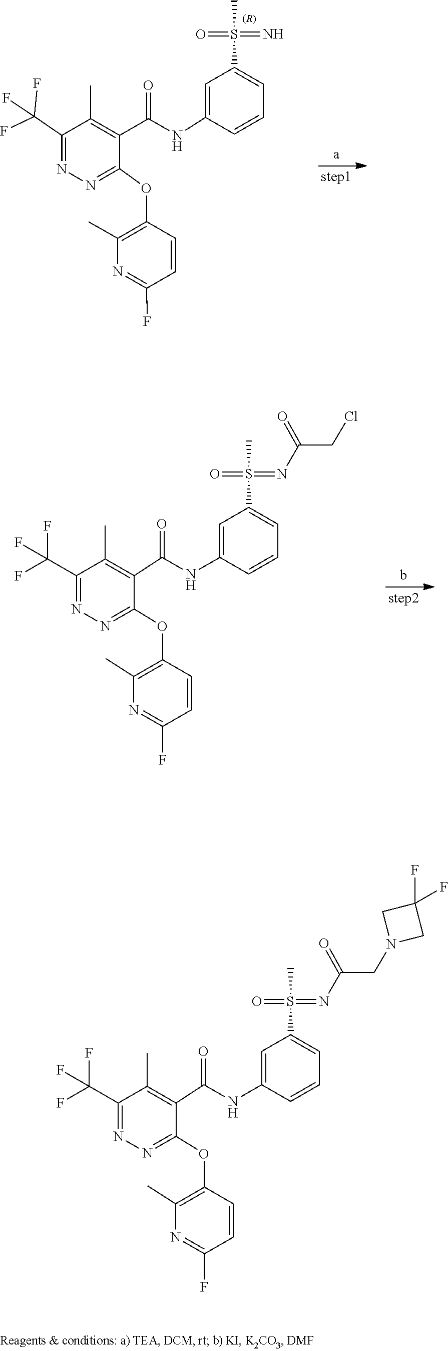

Step 1. (R)—N-(3-(N-(2-chloroacetyl)-S-methylsulfonimidoyl)phenyl)-3-((6-fluoro-2-methylpyridin-3-yl)oxy)-5-methyl-6-(trifluoromethyl)pyridazine-4-carboxamide: A mixture of (R)-3-((6-fluoro-2-methylpyridin-3-yl)oxy)-5-methyl-N-(3-(S-methylsulfonimidoyl)phenyl)-6-(trifluoromethyl)pyridazine-4-carboxamide (300 mg, 0.62 mmol) and TEA (188 mg, 1.86 mmol) in DCM (10 mL) was added 2-chloroacetyl chloride (84 mg, 0.74 mmol) at room temperature. The reaction mixture was stirred at room temperature for 16 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to provide (R)—N-(3-(N-(2-chloroacetyl)-S-methylsulfonimidoyl)phenyl)-3-((6-fluoro-2-methylpyridin-3-yl)oxy)-5-methyl-6-(trifluoromethyl)pyridazine-4-carboxamide 3 (180 mg, 51.8% yield) as a yellow solid. LCMS (ESI) calcd. for C₂₂H₁₉ClF₄N₅O₄S [M+H]⁺ m/z 560.08. found 560.00.

Step 2. (R)—N-(3-(N-(2-(3,3-difluoroazetidin-1-yl)acetyl)-S-methylsulfonimidoyl)phenyl)-3-((6-fluoro-2-methylpyridin-3-yl)oxy)-5-methyl-6-(trifluoromethyl)pyridazine-4-carboxamide: A mixture of (R)—N-(3-(N-(2-chloroacetyl)-S-methylsulfonimidoyl)phenyl)-3-((6-fluoro-2-methylpyridin-3-yl)oxy)-5-methyl-6-(trifluoromethyl)pyridazine-4-carboxamide (180 mg, 0.32 mmol), KI (107 mg, 0.64 mmol) and K₂CO₃ (89 mg, 0.64 mmol) in DMF (5 mL) was added 3,3-difluoroazetidine hydrochloride (60 mg, 0.64 mmol) at room temperature. The reaction mixture was stirred at room temperature for 16 hours. After the reaction was completed, the resulting solution was diluted with water (15 mL) and extracted with EtOAc (15 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 90% MeCN/H₂O containing 0.1% formic acid) to provide (R)—N-(3-(N-(2-(3,3-difluoroazetidin-1-yl)acetyl)-S-methylsulfonimidoyl)phenyl)-3-((6-fluoro-2-methylpyridin-3-yl)oxy)-5-methyl-6-(trifluoromethyl)pyridazine-4-carboxamide (34.7 mg, 17.3% yield) as a white solid. ¹H NMR (400 MHz, DMSO-d₆, ppm) δ 11.46 (s, 1H), 8.42 (s, 1H), 8.07-7.80 (m, 2H), 7.80-7.65 (m, 2H), 7.17 (dd, J=8.7, 3.4 Hz, 1H), 3.70-3.58 (m, 4H), 3.47 (s, 3H), 3.37 (s, 2H), 2.54 (d, J=1.1 Hz, 3H), 2.29 (s, 3H). LCMS (ESI) calcd. for C₂₅H₂₃F₆N₆O₄S [M+H]⁺ m/z 617.14, found 617.00.

### Example 29D

### (R)—N-(3-(N-(2-(3,3-difluoroazetidin-1-yl)acetyl)-S-methylsulfonimidoyl)phenyl)-2-((6-fluoro-2-methylpyridin-3-yl)oxy)-4-methyl-5-(trifluoromethyl)nicotinamide

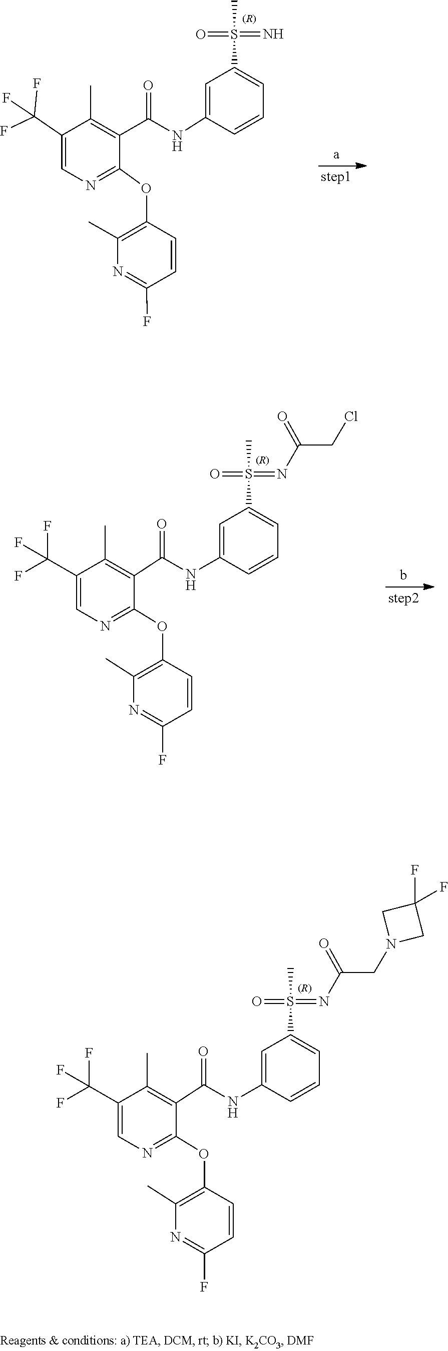

Step 1. (R)—N-(3-(N-(2-chloroacetyl)-S-methylsulfonimidoyl)phenyl)-2-((6-fluoro-2-methylpyridin-3-yl)oxy)-4-methyl-5-(trifluoromethyl)nicotinamide: To a mixture of (R)-2-((6-fluoro-2-methylpyridin-3-yl)oxy)-4-methyl-N-(3-(S-methylsulfonimidoyl)phenyl)-5-(trifluoromethyl)nicotinamide (200 mg, 0.41 mmol), and TEA (84 mg, 0.82 mmol) in DCM (10 mL) was added 2-chloroacetyl chloride (56 mg, 0.50 mmol) at room temperature. The reaction mixture was stirred at room temperature for 16 hours. After the reaction was completed, the resulting solution was diluted with water (30 mL) and extracted with EtOAc (30 mL×3). The combined organic layers were washed with brine, dried over Na₂SO₄, concentrated under vacuum. The residue was purified by flash column chromatography on silica gel (PE/EtAOc=1/1) to provide (R)—N-(3-(N-(2-chloroacetyl)-S-methylsulfonimidoyl)phenyl)-2-((6-fluoro-2-methylpyridin-3-yl)oxy)-4-methyl-5-(trifluoromethyl)nicotinamide (150 mg, 64.7% yield) as a yellow solid. LCMS (ESI) calcd. for C₂₃H₂₀ClF₄N₄O₄S [M+H]⁺ m/z 559.09, found 558.90.

Step 2. (R)—N-(3-(N-(2-(3,3-difluoroazetidin-1-yl)acetyl)-S-methylsulfonimidoyl)phenyl)-2-((6-fluoro-2-methylpyridin-3-yl)oxy)-4-methyl-5-(trifluoromethyl)nicotinamide: To a mixture of (R)—N-(3-(N-(2-chloroacetyl)-S-methylsulfonimidoyl)phenyl)-2-((6-fluoro-2-methylpyridin-3-yl)oxy)-4-methyl-5-(trifluoromethyl)nicotinamide (150 mg, 0.27 mmol), KI (89 mg, 0.54 mmol) and K₂CO₃ (74 mg, 0.54 mmol) in DMF (5 mL) was added 3,3-difluoroazetidine hydrochloride (50 mg, 0.54 mmol) at room temperature. The reaction mixture was stirred at room temperature for 16 hours. After the reaction was completed, the resulting solution was diluted with water (15 mL) and extracted with EtOAc (15 mL×3). The combined organic layers were washed with brine, dried over sodium sulfate, concentrated under vacuum. The residue was purified by prep-HPLC (Gemini 5 um C₁₈ column, 150*21.2 mm, eluting with 50% to 90% McCN/120 containing 0.1% formic acid) to provide (R)—N-(3-(N-(2-(3,3-difluoroazetidin-1-yl)acetyl)-S-methylsulfonimidoyl)phenyl)-2-((6-fluoro-2-methylpyridin-3-yl)oxy)-4-methyl-5-(trifluoromethyl)nicotinamide (55.3 mg, 33.5% yield) as a white solid. ¹H NMR (400 MHz, DMSO-d₆, ppm) δ 11.32 (s, 1H), 8.56 (s, 1H), 8.47 (s, 1H), 7.97-7.79 (m, 2H), 7.75-7.64 (m, 2H), 7.11 (dd, J=8.7, 3.3 Hz, 1H), 3.70-3.58 (m, 4H), 3.47 (s, 3H), 3.37 (s, 2H), 2.51 (s, 3H), 2.24 (s, 3H). LCMS (ESI) calcd. for C₂₆H₂₄F₆N₅4₅S [M+H]⁺ m/z 616.15, found 616.05.

### Example 30D and Example 31D

### (R)-2-((6-fluoro-2-methylpyridin-3-yl)oxy)-4-methyl-N-(3-(1-oxido-3-oxo-4,5-dihydro-3H-1l6-isothiazol-1-yl)phenyl)-5-(trifluoromethyl)nicotinamide & (S)-2-((6-fluoro-2-methylpyridin-3-yl)oxy)-4-methyl-N-(3-(1-oxido-3-oxo-4,5-dihydro-3H-1l6-isothiazol-1-yl)phenyl)-5-(trifluoromethyl)nicotinamide

### Synthetic Route:

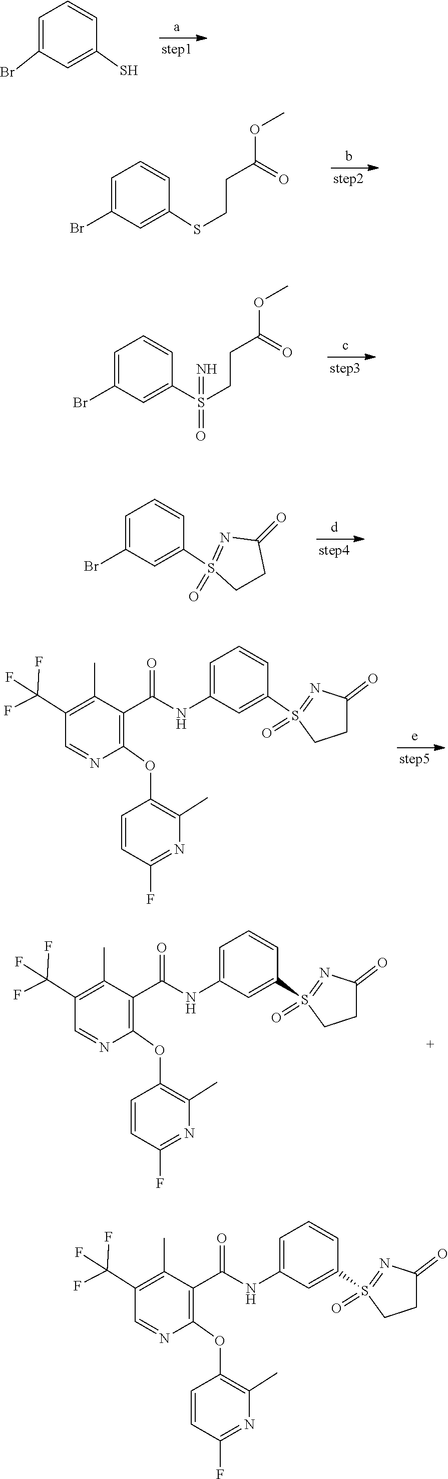

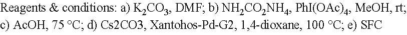

Step 1. methyl 3-((3-bromophenyl)thio)propanoate: A solution of 3-bromobenzenethiol 1 (5 g, 0.03 mol) in DMF (20 mL) was added methyl 3-bromopropanoate (4.9 g, 0.03 mol), and K₂CO₃ (11 g, 0.08 mol) at room temperature. The mixture was stirred at 60° C. for 4 hours. After the reaction was completed, the mixture was cooled to room temperature and the mixture was diluted with water (20 mL) and extracted with DCM (20 mL×3). The combined organic phases were washed with brine, dried over sodium sulfate, concentrated under vacuum. The residue was purified by flash column chromatography on silica gel (PE/EtOAc=1/1) to give methyl 3-((3-bromophenyl)thio)propanoate (4.5 g, 61.64% yield) as a white oil. ¹H NMR (400 MHz, DMSO-d₆, ppm) δ 7.53-7.52 (m, 1H), 7.40-7.37 (m, 1H), 7.33-7.29 (m, 1H), 7.28-7.26 (t, J=8.0 Hz, 1H), 3.60 (s, 3H), 3.20-3.18 (m, 2H), 2.66-2.62 (m, 2H).

Step 2. methyl 3-(3-bromophenylsulfonimidoyl)propanoate: To a solution of methyl 3-((3-bromophenyl)thio)propanoate 2 (4.5 g, 0.016 mol) in MeOH (20 mL) was added PhI(OAc)₂ (1.8 g, 0.024 mol) and ammonium carbamate (10 g, 0.032 mol) at room temperature. The reaction mixture was heated at room temperature for 1 hour. After the reaction was completed, the mixture was cooled to room temperature, diluted with water (10 mL), and extracted with DCM (10 mL×3). The combine organic phases were washed with brine, dried over sodium sulfate, concentrated under vacuum. The combine organic phases were washed with brine, dried over sodium sulfate, concentrated under vacuum. The residue was purified by flash column chromatography on silica gel (PE/EtOAc=1/1) to afford methyl 3-(3-bromophenylsulfonimidoyl)propanoate (4 g, 81.96% yield) as a white solid. LCMS (ESI) calcd. for C₁₀H₁₃BrNO₃S [M+H]⁺ m/z 305.98, found 305.90.

Step 3. 1-(3-bromophenyl)-4,5-dihydro-3H-1l6-isothiazol-3-one 1-oxide: A solution of methyl 3-(3-bromophenylsulfonimidoyl)propanoate 3 (4 g, 0.013 mol) in AcOH (20 mL) was heated at 75° C. for 4 hours. After the reaction was completed, the mixture was cooled to room temperature and the mixture was diluted with water (20 mL) and extracted with DCM (20 mL×3). The combine organic phases were washed with brine, dried over sodium sulfate, concentrated under vacuum. The residue was purified by flash column chromatography on silica gel (PE/EtOAc=1/1) to give 1-(3-bromophenyl)-4,5-dihydro-3H-1l6-isothiazol-3-one 1-oxide (3 g, 85.71% yield) as a white solid. ¹H NMR (400 MHz, DMSO, ppm) δ 8.12 (t, J=1.7 Hz, 1H), 8.09-8.03 (m, 1H), 7.98 (dd, J=8.0, 0.8 Hz, 1H), 7.69 (t, J=8.0 Hz, 1H), 4.18 (ddd, J=14.4, 10.6, 6.9 Hz, 1H), 3.93 (ddd, J=14.2, 9.2, 4.6 Hz, 1H), 3.09-2.93 (m, 2H).

Step 4. 2-((6-fluoro-2-methylpyridin-3-yl)oxy)-4-methyl-N-(3-(1-oxido-3-oxo-4,5-dihydro-3H-1l6-isothiazol-1-yl)phenyl)-5-(trifluoromethyl)nicotinamide: A solution of 1-(3-bromophenyl)-4,5-dihydro-3H-1l6-isothiazol-3-one 1-oxide (600 mg, 2.20 mmol) and 2-((6-fluoro-2-methylpyridin-3-yl)oxy)-4-methyl-5-(trifluoromethyl)nicotinamide (600 mg, 1.81 mmol) in dioxane (10 mL) was added cesium carbonate (1780 mg, 5.43 mmol) and Xantphos-Pd-G2 (162 mg, 0.18 mmol) at room temperature. The reaction mixture was stirred at 100° C. for 16 hours under nitrogen. The reaction was monitored by LCMS. After the reaction was completed, the mixture was diluted with water (20 mL) and extracted with DCM (20 mL×3). The combined organic phases were washed with brine, dried over sodium sulfate, concentrated under vacuum. The residue was purified by flash column chromatography on silica gel (PE/EtOAc=1/1) to give 2-((6-fluoro-2-methylpyridin-3-yl)oxy)-4-methyl-N-(3-(1-oxido-3-oxo-4,5-dihydro-3H-116-isothiazol-1-yl)phenyl)-5-(trifluoromethyl)nicotinamide (250 mg, 26.56% yield) as a yellow solid. LCMS (ESI) calcd. for C₂₃H₁₉F₄N₄O₄S [M+H]⁺ m/z 523.11, found 523.00.

Step 5. 2-((6-fluoro-2-methylpyridin-3-yl)oxy)-4-methyl-N-(3-(1-oxido-3-oxo-4,5-dihydro-3H-1l6-isothiazol-1-yl)phenyl)-5-(trifluoromethyl)nicotinamide was further purified by Chiral-Prep-HPLC (DAICEL IH, 4.6*250 mm, eluting with 80/20 CO₂/MeOH containing 0.1% NH₃) to afford (S)-2-((6-fluoro-2-methylpyridin-3-yl)oxy)-4-methyl-N-(3-(1-oxido-3-oxo-4,5-dihydro-3H-1l6-isothiazol-1-yl)phenyl)-5-(trifluoromethyl)nicotinamide and (R)-2-((6-fluoro-2-methylpyridin-3-yl)oxy)-4-methyl-N-(3-(1-oxido-3-oxo-4,5-dihydro-3H-1l6-isothiazol-1-yl)phenyl)-5-(trifluoromethyl)nicotinamide.

Example 30D: First eluting compound (43.00 mg, 99.70% purity): ¹H NMR (400 MHz, DMSO-d₆, ppm) δ 11.37 (s, 1H), 8.56 (s, 1H), 8.42 (t, J=1.8 Hz, 1H), 8.05 (d, J=8.1 Hz, 1H), 7.86-7.68 (m, 3H), 7.12 (dd, J=8.7, 3.3 Hz, 1H), 4.22-4.09 (m, 1H), 3.95 (dd, J=9.6, 4.7 Hz, 1H), 3.05 (dd, J=9.5, 6.7 Hz, 1H), 2.93 (dd, J=10.8, 4.9 Hz, 1H), 2.51 (s, 3H), 2.24 (s, 3H). LCMS (ESI) calcd. for C₂₃H₁₉F₄N₄O₄S [M+H]⁺ m/z 523.11, found 523.00.

Example 31D: Second eluting compound (45.00 mg, 98.0% purity): ¹H NMR (400 MHz, DMSO-d₆, ppm) δ 11.38 (s, 1H), 8.56 (s, 1H), 8.43 (s, 1H), 8.05 (d, J=8.0 Hz, 1H), 7.87-7.69 (m, 3H), 7.12 (d, J=8.6 Hz, 1H), 4.21-4.09 (m, 1H), 3.95 (ddd, J=13.8, 9.2, 4.5 Hz, 1H), 3.11-3.02 (m, 1H), 2.99-2.88 (m, 1H), 2.51 (s, 3H), 2.25 (s, 3H). LCMS (ESI) calcd. for C₂₃H₁₉F₄N₄O₄S [M+H]⁺ m/z 523.11, found 523.00.

### Example 32D

### Compound Profiling Human NaV1.8/β3 Cell Line—SyncroPatch384PE Assay

Compounds were tested on recombinant human Nav1.8/b3 stably transfected CHO cells using the SyncroPatch384PE system (Nanion Technologies), an automated patch clamp device. Cells were cultured at 37° C./5% CO₂ in Ham's F-12 supplemented with 10% fetal bovine serum, 100 U/mL penicillin G sodium, 100 mg/mL streptomycin sulfate and selection antibiotics (0.01 mg/ml Blasticidin, 0.4 mg/ml Zeocin and 0.25 mg/ml Hygromycin). On the day of the recordings, cells in culture dishes were washed twice with Hank's Balanced Salt Solution (HBSS) and treated with Accutase for approximately 20 minutes. Immediately before use in the SynchroPatch384PE using an 8-hole NPC-384 chip, the cells were washed in HBSS to remove the Accutase and resuspended in extracellular solution. All experiments were performed at ambient temperature. Intracellular solution contained (mM): CsCl, 50; CsF, 90; MgCl₂, 5; EGTA, 1; HEPES, 10; pH adjusted to 7.2 with CsOH. Extracellular solution contained (mM): NaCl, 137; KCl, 4.0; CaCl₂), 3.8; MgCl₂, 1; HEPES, 10; Glucose, 10; pH adjusted to 7.4 with NaOH. 100 nM tetrodotoxin (TTX) was added to the extracellular solution to block endogenous TTX-sensitive sodium currents.

Compounds were tested in quadruplicate in 0.3% DMSO and 0.03% pluronic Acid. Compounds were diluted 1.3.33 in extracellular solution to create an 8-point concentration response curve. Each plate contained a historical positive control and up to ten compounds. 300 μM tetracaine and 0.3% DMSO+0.03% pluronic acid were used as high and low controls respectively.

Whole cell patch clamp recordings were conducted according to Nanion's standard procedure for SyncroPatch384PE®. Cells were held at a holding potential of −120 mV. A depolarization step to 10 mV for 30 ms was applied (P1 measurement), followed by a hyperpolarization step to −100 mV for 100 ms. An inactivation step at −35 mV for 10 sec was applied before stepping to −100 mV for 20 ms, followed by a step to 10 mV for 30 ms (P2 measurement) and then back to −100 mV for 30 ms. Sweep interval was 15 sec. Following establishment of the whole-cell configuration in extracellular solution, cells were washed in extracellular solution containing 0.3% DMSO and 0.03% pluronic acid to stabilize the baseline current. Compounds were then applied by the SynchroPatch384 PE system into each well and the current was recorded for five minutes in extracellular solution, followed by application of tetracaine to achieve full block at the end of experiment. The potency of the compounds was assessed on two read-outs, resting state block (P1 measurement) or inactivated state block (P2 measurement) to obtain IC50 values. Values were normalized to high (tetracaine) and low (DMSO+pluronic acid) controls.

The table below shows the potency of compounds against human Nav1.8, where “A” represents an IC50 less than or equal to 5 nM, “B” represents an IC50 greater than 5 nM to less than or equal to 50 nM, “C” represents an IC50 greater than 50 nM to less than or equal to 100 nM, “D” represents an IC₅₀ greater than 100 nM to less than or equal to 200 nM, “E” represents an IC₅₀ greater than 200 nM.

| Example | P1 | P2 |
|---|---|---|
| 1D | A | A |
| 2D | A | A |
| 3D | A | A |
| 4D | A | A |
| 5D | A | A |
| 6D | A | A |
| 7D | A | A |
| 8D | A | A |
| 9D | A | A |
| 10D | A | A |
| 11D | A | A |
| 12D | A | A |
| 13D | A | A |
| 14D | A | A |
| 15D | A | A |
| 16D | A | A |
| 17D | A | A |
| 18D | A | A |
| 19D | B | B |
| 20D | B | B |
| 21D | A | A |
| 22D | A | A |
| 23D | A | A |
| 24D | B | B |
| 25D | A | A |
| 26D | A | A |
| 27D | A | A |
| 28D | A | A |
| 29D | A | A |

### Example 33D

### Compound Profiling Human NaV1.8/β1 Cell Line—Sophion QPatch II Assay

Compounds were tested on recombinant human Nav1.8/β1 stably transfected HEK293 (Eurofins, CYL3025, St. Charles, MO) cells using the QPatch II system (Sophion Bioscience A/S, Ballerup—Denmark), an automated patch clamp device. Cells were cultured at 37° C./5% CO₂ in DMEM/F-12 supplemented with 10% fetal bovine serum, lx Non-Essential Amino Acids, and selection antibiotics (0.625 μg/mL Puromycin, 400 μg/mL Geneticin, and 100 μg/mL Hygromycin). One or two days prior to recording, cells were moved to a lower temperature 30° C./5% CO₂ incubator to increase surface expression of the channels. On the day of the recordings, cells in culture dishes were washed twice with Ca²⁺—Mg²⁺ free DPBS and treated with Detachin (Genlantis, San Diego) for 3 minutes and then resuspended in Serum Free Medium (EX-CELL® ACF CHO Medium for Cell Culture (Sigma-Aldrich) supplemented with 0.04 mg/mL soybean trypsin Inhibitor and 25 mM HEPES (all components supplied by Sigma-Aldrich, St. Louis, MO) and allowed to recover for a minimum of 20 minutes in the onboard QStirrer. All experiments were performed at ambient temperature. Intracellular solution contained (mM): CsCl, 50; CsF, 90; MgCl₂, 2; EGTA, 5; HEPES, 10; pH adjusted to 7.2 with CsOH. Extracellular solution contained (mM): NaCl, 137; KCl, 4; CaCl₂), 3.8; MgCl₂, 1; HEPES, 10; Glucose, 10; pH adjusted to 7.4 with NaOH. 500 nM tetrodotoxin (TTX) was added to the extracellular solution to block endogenous TTX-sensitive sodium currents.

Compounds were tested in an interleaved manner across two cohorts of wells to develop an aggregate 8-point concentration response curve. Final concentrations include 0.1% DMSO and 0.03% pluronic Acid. In general, the concentration range spans a range from 0.01-300 nM. Compounds were diluted 1:10 with extracellular solution within their respective cohort. DMSO (0.1% by volume) served as a vehicle control to ascertain assay performance. Whole cell patch clamp recordings were conducted according to Sophion standard procedure for QPatch II®. Cells were held at a holding potential of −120 mV. A depolarizing step to 10 mV for 30 ms was applied to elicit inward Na⁺ currents. Inter-sweep interval was 15 sec. Following establishment of the whole-cell configuration in extracellular solution, cells were washed twice in extracellular solution containing 0.1% DMSO and 0.03% pluronic acid to stabilize the baseline current. Compounds were then applied by the QPatch II system into each well in an ascending manner within cohort and the current was recorded for five minutes for each concentration. Percent of current remaining was determined by taking the average of the last 5 sweeps within each concentration and normalizing to the latest pre-compound saline period. The potency (IC₅₀) values of the compounds were only assessed against resting state block. Wells whose initial sodium current magnitude were below 2 nA or whole cell resistance fell below 100 MΩ were excluded from analysis.

The table below shows the potency of compounds against human Nav1.8, where “A” represents an IC₅₀ less than or equal to 5 nM, “B” represents an IC₅₀ greater than 5 nM to less than or equal to 50 nM, “C” represents an IC₅₀ greater than 50 nM to less than or equal to 100 nM, “D” represents an IC₅₀ greater than 100 nM to less than or equal to 200 nM, “E” represents an IC₅₀ greater than 200 nM.

| Example | P1 |
|---|---|
| 30D* | E |
| 31D* | C |

In still other embodiments, the invention provides methods of treating a condition in a subject by providing to a subject having a condition a compound of the invention, such as any of those described above.

### Compositions

The invention provides pharmaceutical compositions containing compounds of the inventions, such as those described above. The pharmaceutical composition may be in a form suitable for oral use, for example, as tablets, troches, lozenges, fast-melts, aqueous or oily suspensions, dispersible powders or granules, emulsions, hard or soft capsules, syrups, or elixirs. Compositions intended for oral use may be prepared according to any method known in the art for the manufacture of pharmaceutical compositions and such compositions may contain one or more agents selected from sweetening agents, flavoring agents, coloring agents, and preserving agents, to provide pharmaceutically elegant and palatable preparations. Tablets contain the compounds in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch, or alginic acid; binding agents, for example starch, gelatin or acacia, and lubricating agents, for example magnesium stearate, stearic acid, or talc. The tablets may be uncoated, or they may be coated by known techniques to delay disintegration in the stomach and absorption lower down in the gastrointestinal tract and thereby provide a sustained action over a longer period. For example, a time delay material such as glyceryl monostearate or glyceryl distearate may be employed. They may also be coated by the techniques described in U.S. Pat. Nos. 4,256,108; 4,166,452; and 4,265,874, the contents of which are incorporated herein by reference, to form osmotic therapeutic tablets for control release. Preparation and administration of compounds is discussed in U.S. Pat. No. 6,214,841 and U.S. Pub. No. 2003/0232877, the contents of which are incorporated herein by reference.

Formulations for oral use may also be presented as hard gelatin capsules in which the compounds are mixed with an inert solid diluent, for example calcium carbonate, calcium phosphate or kaolin, or as soft gelatin capsules in which the compounds are mixed with water or an oil medium, for example peanut oil, liquid paraffin, or olive oil.

An alternative oral formulation, where control of gastrointestinal tract hydrolysis of the compound is sought, can be achieved using a controlled-release formulation, where a compound of the invention is encapsulated in an enteric coating.

Aqueous suspensions may contain the compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents such as a naturally occurring phosphatide, for example lecithin, or condensation products of an alkylene oxide with fatty acids, for example, polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such a polyoxyethylene with partial esters derived from fatty acids and hexitol anhydrides, for example polyoxyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the compounds in a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the compounds in admixture with a dispersing or wetting agent, suspending agent and one or more preservatives. Suitable dispersing or wetting agents and suspending agents are exemplified, for example sweetening, flavoring, and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavoring agents.

Syrups and elixirs may be formulated with sweetening agents, such as glycerol, propylene glycol, sorbitol, or sucrose. Such formulations may also contain a demulcent, a preservative, and agents for flavoring and/or coloring. The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer [symbol] solution, and isotonic sodium chloride solution. In addition, sterile, fixed oils are conventionally employed as a solvent or suspending medium. For this purpose, any bland fixed oil may be employed, including synthetic mono- or di-glycerides. In addition, fatty acids such as oleic acid find use in the preparation of injectables.

In certain embodiments, the formulation is a sustained release formulation. In certain embodiments, the formulation is not a sustained release formulation. In certain embodiments, the formulation is not injectable. In certain embodiments, the formulation does not contain particles having a D50 (volume weighted median diameter) of less than 10 microns. In certain embodiments, the formulation does not contain a polymer surface stabilizer. In certain embodiments, the formulation is not an aqueous suspension.

The composition may be formulated for administration by a particular mechanism. The composition may be formulated for oral, intravenous, enteral, parenteral, dermal, buccal, topical, nasal, or pulmonary administration. The composition may be formulated for administration by injection or on an implantable medical device (e.g., stent or drug-eluting stent or balloon equivalents).

The composition may be formulated a single daily dosage. The composition may be formulated for multiple daily dosages, e.g., two, three, four, five, six or more daily dosages.

### Methods of Treating Conditions

The invention provides methods of treating a condition in a subject using compounds of the invention. The methods are useful for treating any condition associated with aberrant, e.g., increased, activity of voltage gated sodium channel Nav1.8. Conditions associated with increased activity at voltage gated sodium channels and the use of inhibitors to treat such conditions is known in the art and described in, for example, International Patent Publication Nos. WO 2020/014243, WO 2020/014246, WO 2020/092187, the contents of each of which are incorporated herein by reference.

For example and without limitation, the condition may be abdominal cancer pain, acute cough, acute idiopathic transverse myelitis, acute itch, acute pain, acute pain in major trauma/injury, airways hyperreactivity, allergic dermatitis, allergies, ankylosing spondylitis, asthma, atopy, Behcet© disease, bladder pain syndrome, bone cancer pain, brachial plexus injury, burn injury, burning mouth syndrome, calcium pyrophosphate deposition disease, cervicogenic headache, Charcot neuropathic osteoarthropathy, chemotherapy-induced oral mucositis, chemotherapy-induced peripheral neuropathy, cholestasis, chronic cough, chronic itch, chronic low back pain, chronic pain, chronic pancreatitis, chronic post-traumatic headache, chronic widespread pain, cluster headache, complex regional pain syndrome, complex regional pain syndromes, constant unilateral facial pain with additional attacks, contact dermatitis, cough, dental pain, diabetic neuropathy, diabetic peripheral neuropathy, diffuse idiopathic skeletal hyperostosis, disc degeneration pain, distal sensory polyneuropathy (DSP) associated with highly active antiretroviral therapy (HAART), Ehlers-Danlos syndrome, endometriosis, epidermolysis bullosa, epilepsy, erythromelalgia, Fabry© disease, facet joint syndrome, failed back surgery syndrome, familial hemiplegic migraine, fibromyalgia, glossopharyngeal neuralgia, glossopharyngeal neuropathic pain, gout, head and neck cancer pain, inflammatory bowel disease, inflammatory pain, inherited erythromelalgia, irritable bowel syndrome, irritable bowel syndrome, itch, juvenile idiopathic arthritis, mastocytosis, melorheostosis, migraine, multiple sclerosis, musculoskeletal damage, myofascial orofacial pain, neurodegeneration following ischemia, neurofibromatosis type II, neuropathic ocular pain, neuropathic pain, neuropathic pain, nociceptive pain, non-cardiac chest pain, optic neuritis, oral mucosal pain, orofacial pain, osteoarthritis, osteoarthritis, overactive bladder, pachyonychia congenita, pain, pain resulting from cancer, pain resulting from chemotherapy, pain resulting from diabetes, pain syndrome, painful joint arthroplasties, pancreatitis, Parkinson [symbol] disease, paroxysmal extreme pain disorder, pemphigus, perioperative pain, peripheral neuropathy, persistent idiopathic dentoalveolar pain, persistent idiopathic facial pain, phantom limb pain, phantom limb pain, polymyalgia rheumatica, postherpetic neuralgia, post-mastectomy pain syndrome, postoperative pain, post-stroke pain, post-surgical pain, post-thoracotomy pain syndrome, post-traumatic stress disorder, preoperative pain, pruritus, psoriasis, psoriatic arthritis, pudendal neuralgia, pyoderma gangrenosum, radiotherapy-induced peripheral neuropathy, Raynaud [symbol] disease, renal colic, renal colic, renal failure, rheumatoid arthritis, salivary gland pain, sarcoidosis, sciatica, scleroderma, sickle cell disease, small fiber neuropathy, spinal cord injury pain, spondylolisthesis, spontaneous pain, stump pain, subacute cough, temporomandibular joint disorders, tension-type headache, trigeminal neuralgia, vascular leg ulcers, vulvodynia, or whiplash associated disorder.

Methods of treating a condition in a subject may include providing a composition of the invention to a subject. The composition may be provided to a subject by any suitable route or mode of administration. For example and without limitation, the composition may be provided buccally, dermally, enterally, intraarterially, intramuscularly, intraocularly, intravenously, nasally, orally, parenterally, pulmonarily, rectally, subcutaneously, topically, transdermally, by injection, or with or on an implantable medical device.

The composition may be provided according to a dosing regimen. A dosing regimen may include one or more of a dosage, a dosing frequency, and a duration.

Doses may be provided at any suitable interval. For example and without limitation, doses may be provided once per day, twice per day, three times per day, four times per day, five times per day, six times per day, eight times per day, once every 48 hours, once every 36 hours, once every 24 hours, once every 12 hours, once every 8 hours, once every 6 hours, once every 4 hours, once every 3 hours, once every two days, once every three days, once every four days, once every five days, once every week, twice per week, three times per week, four times per week, or five times per week.

The dose may be provided in a single dosage, i.e., the dose may be provided as a single tablet, capsule, pill, etc. Alternatively, the dose may be provided in a divided dosage, i.e., the dose may be provided as multiple tablets, capsules, pills, etc.

The dosing may continue for a defined period. For example and without limitation, doses may be provided for at least 1 week, at least 2 weeks, at least 3 weeks, at least 4 weeks, at least 6 weeks, at least 8 weeks, at least 10 weeks, at least 12 weeks, at least 4 months, at least 5 months, at least 6 months, at least 8 months, at least 10 months, at least 12 months or more.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification, and guidance that can be adapted to the practice of this invention in its various embodiments and equivalents thereof.

1. A compound of formula (I): 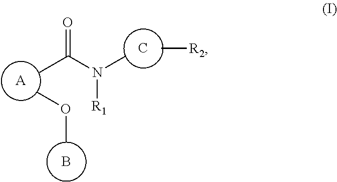
and pharmaceutically acceptable salts, hydrates and solvates thereof, wherein:
A is a substituted or unsubstituted heteroaryl ring comprising at least one heteroatom selected from a group consisting of O, S, or N;
wherein the one or more substitutions on A are selected from H, —OH, halo, C₁-C₈-alkyl, C₁-C₈ fully or partially fluorinated fluoroalkyl, C₂-C₈ branched alkyl, alkenyl, alkynyl, haloalkyl, alkoxy, cycloalkoxy, haloalkoxy, nitro, cyano, —C(R′)(R″)-cycloalkyl, C(R′)(R″)-aryl, —NR′R″, substituted or unsubstituted 3-8 membered cycloalkyl, 3-8 membered cycloalkenyl, or 3-6 membered heterocycloalkyl, wherein the 3-6 membered heterocycloalkyl comprises at least one heteroatom independently selected from O, S, and N;
B is substituted or unsubstituted aryl or heteroaryl, wherein the substitutions are selected from the group consisting of substituted or unsubstituted C₁-C₈ alkyl, deuterated C₁-C₄ alkyl wherein the alkyl chain may be fully or partially deuterated, halo-C₁-C₄ alkyl where the alkyl chain is fully or partially halogenated, C₃-C₁₀ cycloalkyl, halogen, cyano, nitro, C₁-C₈ alkoxyl, haloalkoxyl wherein the haloalkyoxyl chain may be fully or partially halogenated, or arylalkoxyl;
C is substituted or unsubstituted aryl or heteroaryl, wherein the one or more substitutions are independently selected from a group consisting of halo, C₁-C₈ alkyl, haloalkyl, alkoxy;
R₁ is selected from a group consisting of H and C₁-C₄ alkyl;
R₂ is selected from a group consisting of: Formula (II): 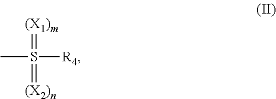
C(═O)NH₂, and —C(═O)NR′R″;
wherein,
m and n are independently 0 or 1; and
X₁ is O and X₂ is either NH or NR′; or
X₁ and X₂ are both O, NH, or NR′; or
X₁ is O and X₂ is NR₃; wherein R₃ is selected from a group consisting of: CD₃, C₁-C₄ alkyl or cycloalkyl, haloalkyl, halocycloalkyl, aryl, heteroaryl, and heterocyclyl, (C₁-C₆)alkyl-OH, (C₁-C₆)alkyl-NHR′, (C₁-C₆)alkyl-NR′R″, (C₁-C₆)alkyl-O—(C₁-C₆)alkyl, (C₁-C₆)alkyl-N—(C₁-C₆)alkyl;
R₄ is selected from a group consisting of: —NH₂, —NR′R″, C₁-C₄ alkyl, C₃-C₈-cycloalkyl, haloalkyl, halocycloalkyl, aryl, heteroaryl, and heterocyclyl.

2. The compound of claim 1, wherein B is a 6 membered substituted or unsubstituted heteroaryl ring, comprising one or more N atoms.

3. The compound of claim 2, wherein the one or more N atoms in the heteroaryl ring are in form of a N-oxide.

4. The compound of claim 2, wherein the B ring including the N-oxide is selected from group consisting of: pyridyl N-oxide, pyrazinyl N-oxide, and pyrimidinyl N-oxide.

5. The compound of claim 1, wherein the compound is a compound of formula (III): 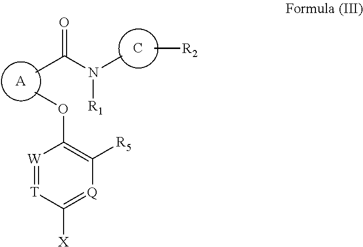
wherein:
Q, T and W are independently N or CR₆;
R₆ is H, halogen, —CD₃, alkyl, cycloalkyl, haloalkyl, alkoxy, haloalkoxy, cyano, —CF₃, —OCF₃, or substituted or unsubstituted cycloalkoxy;
R₅ is H, —OH, halo, —CD₃, C₁-C₆-alkyl, branched alkyl, haloalkyl where the alkyl chain is fully or partially halogenated, alkoxy, arylalkoxy, cycloalkoxy, haloalkoxy, cyano, —CH₂-cycloalkyl, —CH(CH₃)-cycloalkyl, trifluoromethyl, cyclopropylmethyl, substituted or unsubstituted 3-6 membered cycloalkyl, any of which may have one or more substituents;
X is H, halo, —CD₃, alkyl, haloalkyl, alkoxy, cycloalkoxy, haloalkoxy, cyano, —CF₃, —OCF₃.

6. The compound of claim 1, wherein the compound is a compound of Formula (IV): 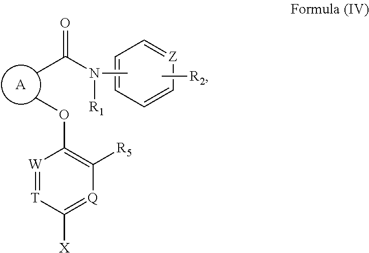
wherein:
Z is CR₁₀, N, or N⁺O⁻; wherein
R₁₀ is H, halo, —CD₃, C₁-C₈ alkyl, haloalkyl, or alkoxy.

7. The compound of claim 1, wherein A is substituted or unsubstituted 6 or more membered heteroaryl ring having at least one heteroatom independently selected from N, O, or S.

8. The compound of claim 1, wherein A is substituted or unsubstituted pyridyl, pyrimidinyl, pyrazinyl, or pyridazinyl.

9. The compound of claim 1, wherein A is substituted or unsubstituted 6 membered heteroaryl having at least one heteroatom, wherein the heteroatom is N.

10. The compound of claim 1, wherein A is 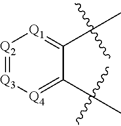
wherein Q₁, Q₂, Q₃ and Q₄ are independently selected from a group consisting of: N, N⁺O⁻, or CR₇; wherein at least two of Q₁, Q₂, Q₃ and Q₄ are CR₇;
R₇ is H, —OH, halo, —CD₃, alkyl, haloalkyl, alkoxy, haloalkoxy, cyano, —CF₃, —OCF₃, substituted or unsubstituted 5 or 6 membered ring heterocyclyl or heteroaryl, saturated heterocyclyl, or partially unsaturated heterocyclyl, O-aryl, O-heteroaryl, O-cycloalkyl, or O-cycloheteroalkyl.

11. The compound of claim 1, wherein A is 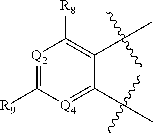
wherein,
Q₂ and Q₄ are independently N or N⁺O⁻;
Q₂ is N or N⁺O⁻; Q₄ is CR₇; or
Q₂ is CR₇, Q₄ is N or N⁺O⁻;
R₈ and R₉ are independently selected from a group consisting of H, —OH, halo, —CD₃, substituted or unsubstituted C₁-C₆ alkyl, branched alkyl, alkenyl, alkylnyl, haloalkyl, alkoxy, cycloalkyl, heterocyclyl, NH₂, NHR′, NR′R″, aryl, heteroaryl, —CF₂CH₃, and —CF₂CF₃;
R₇ is described in claim 10.

12. The compound of claim 1, wherein A is 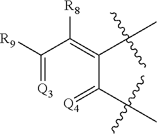
wherein,
Q₃ and Q₄ are independently N or N⁺O⁻;
Q₃ is N or N⁺O⁻ and Q₄ is CR₇; or
Q₃ is CR₇, Q₄ is N or N⁺O⁻; wherein R₇, R₈ and R₉ are defined above.

13. The compound of claim 1, wherein A is 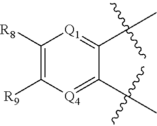
wherein,
Q₁ and Q₄ are independently N or N⁺O⁻;
Q₁ is N or N⁺O⁻, and Q₄ is CR₇;
Q₁ is CR₇ and Q₄ is N or N⁺O⁻; or
Q₁ or Q₄ is CR₇; wherein R₇, R₈, and R₉ are defined above.

14. The compound of claim 1, wherein A is 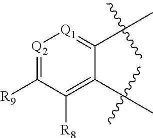
wherein Q₁ and Q₂ are N; wherein R₈ and R₉ are defined above.

15. The compound of claim 1, wherein A is 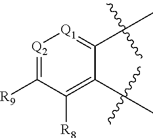
wherein Q₁ is CR₇, Q₂ is N; R₇, R₈, and R₉ are defined above.

16. The compound of claim 1, wherein A is 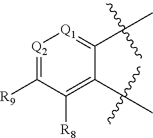
wherein, Q₁ is N; Q₂ is CR₇; R₇, R₈, and R₉ are defined above.

17. The compound of claim 1, wherein R₁ is H.

18. The compound of claim 1, wherein R₂ is: 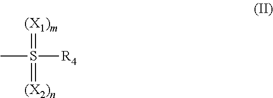
wherein m and n are independently 0 or 1;
X₁ is O and X₂ is NH, and R₄ is alkyl.

19. The compound of claim 1, wherein R₂ is: 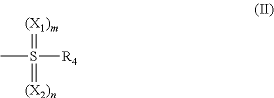
wherein m and n are independently 0 or 1;
X₁ is O and X₂ is O, and R₄ is NH₂ or alkyl, for example, methyl.

20. The compound of claim 1, wherein R₂ is —S(═O)CH₃.

21. The compound of claim 5, wherein, Q is N or CH; R₅ is methyl or —OMe, X is F or CN.

22. The compound of claim 5, wherein Q is CF.

23. The compound of claim 6, wherein R₁₀ is H or F.

24. The compound of claim 6, wherein Z is N or CH.

25. The compound of claim 6, wherein Z is CF.

26. The compound of any one of claims 11-16, wherein R₇, R₈, and R₉ are independently selected from a group consisting of: H, methyl, fluoro, chloro, bromo, CF₃, cyclopropyl, difluorophenyl, and dimethylpyrazole.

27. The compound of claim 1, wherein R₂ does not comprise —C(═O)NH₂ or —C(═O)NR′R″.

28. The compound of claim 1, wherein the compound is selected from a group consisting of:

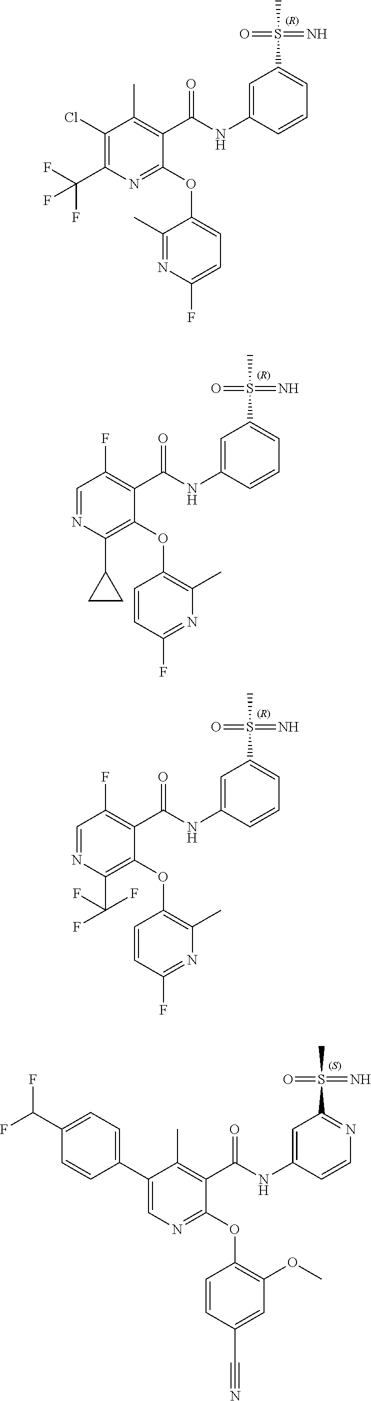

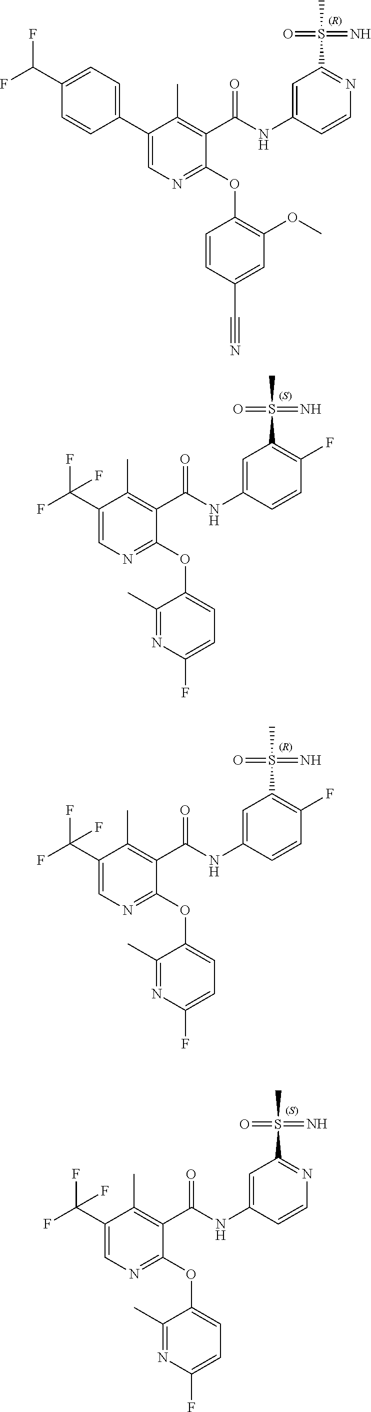

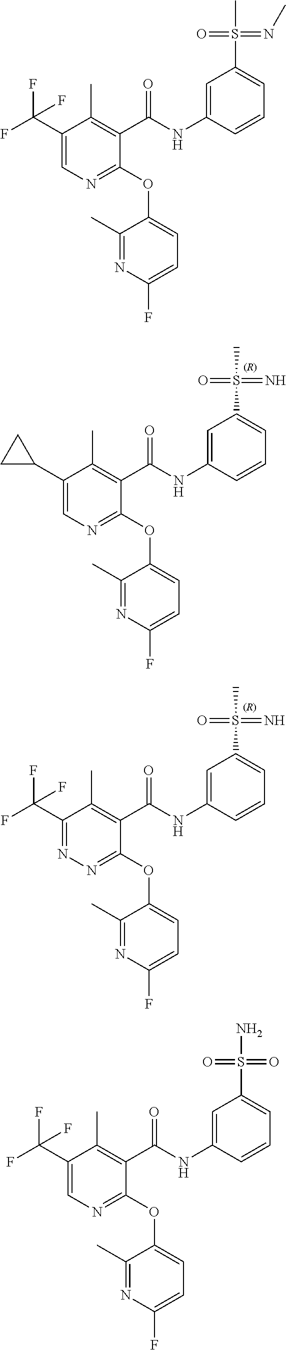

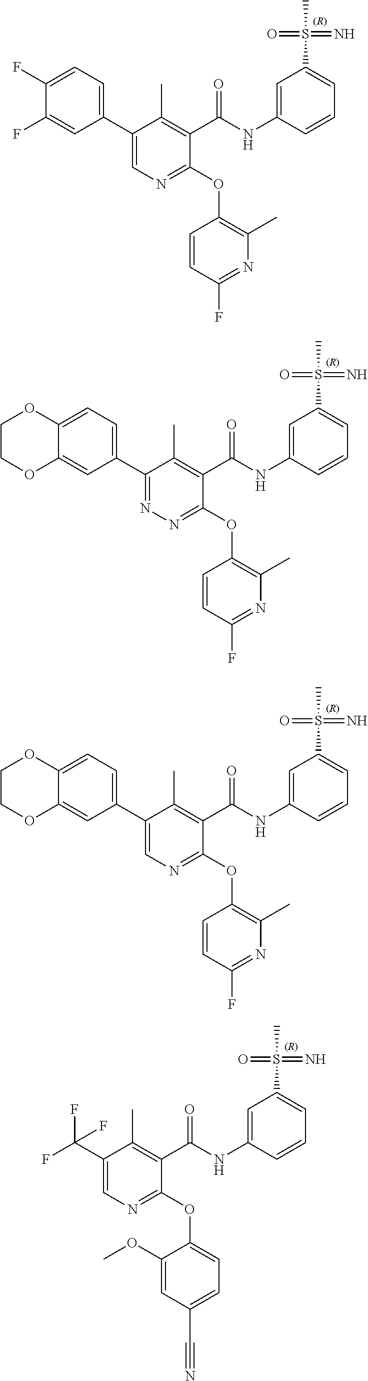

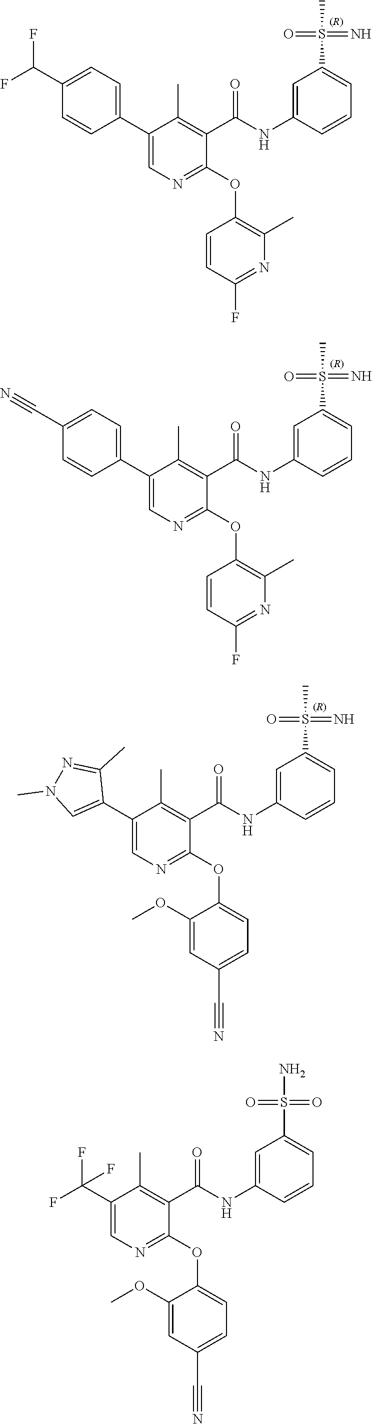

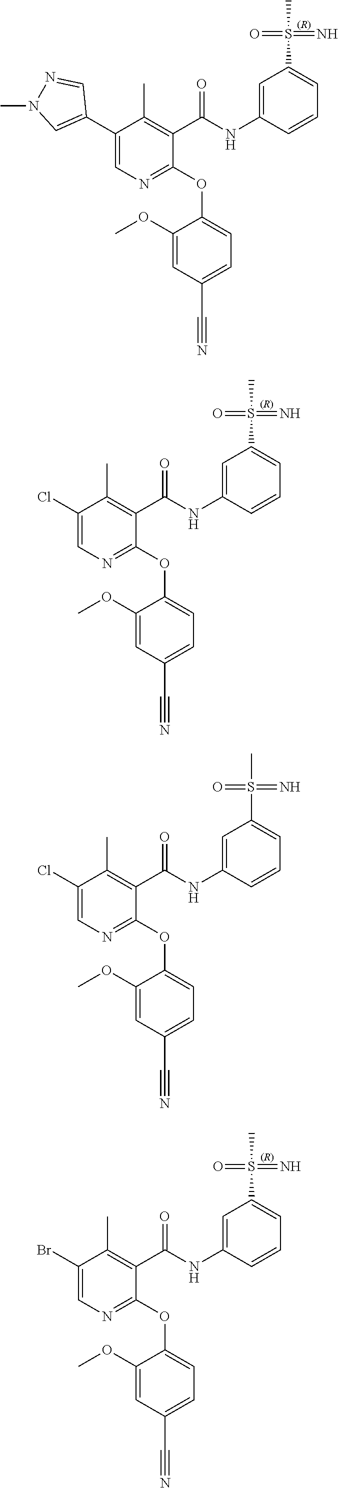

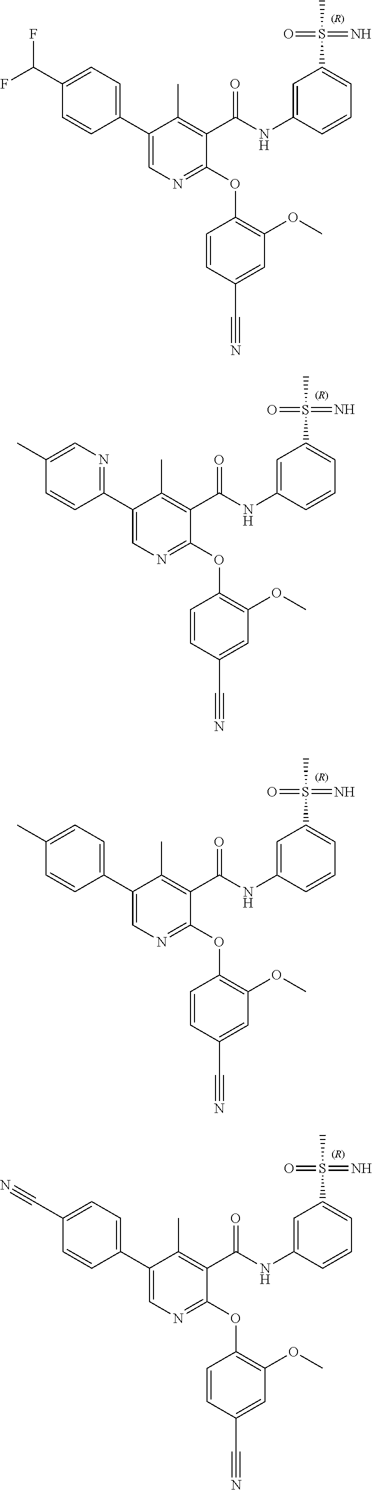

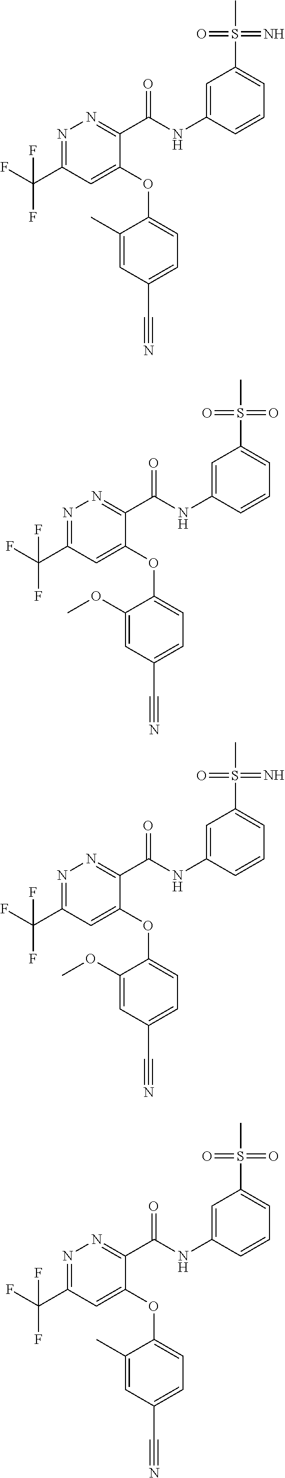 [SMILES: Cc1cc(C#N)ccc1Oc1cc(C(F)(F)F)nnc1C(=O)Nc1cccc(S(C)(=N)=O)c1 ; COc1cc(C#N)ccc1Oc1cc(C(F)(F)F)nnc1C(=O)Nc1cccc(S(C)(=O)=O)c1 ; COc1cc(C#N)ccc1Oc1cc(C(F)(F)F)nnc1C(=O)Nc1cccc(S(C)(=N)=O)c1 ; Cc1cc(C#N)ccc1Oc1cc(C(F)(F)F)nnc1C(=O)Nc1cccc(S(C)(=O)=O)c1]

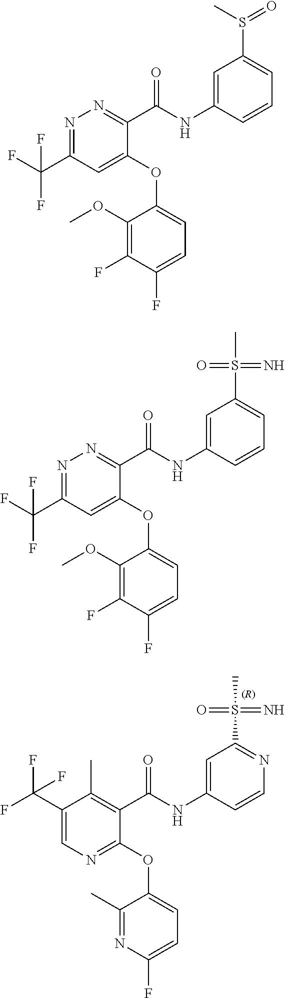

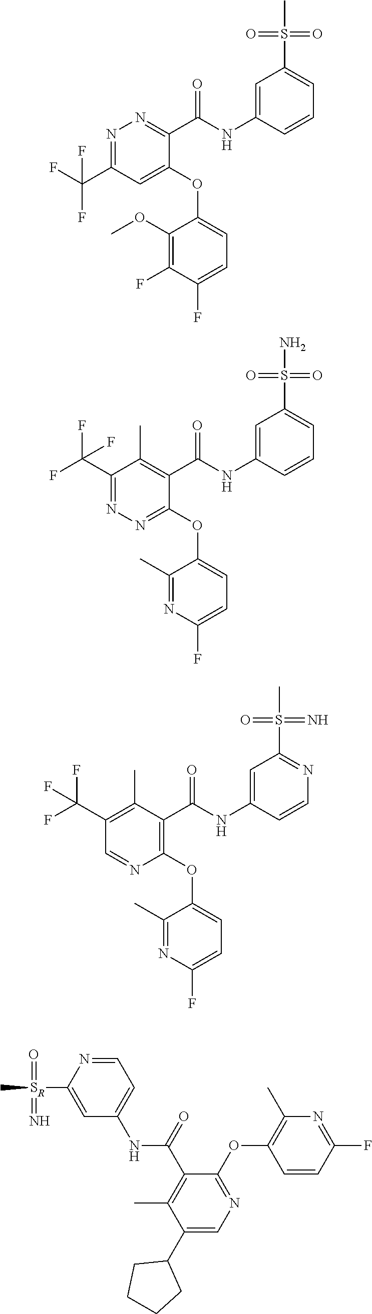

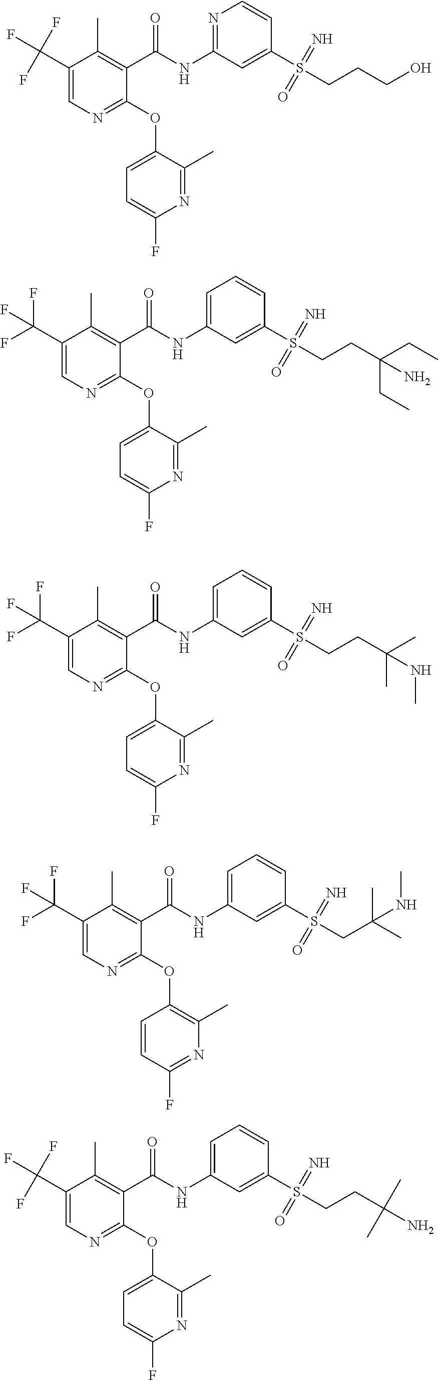

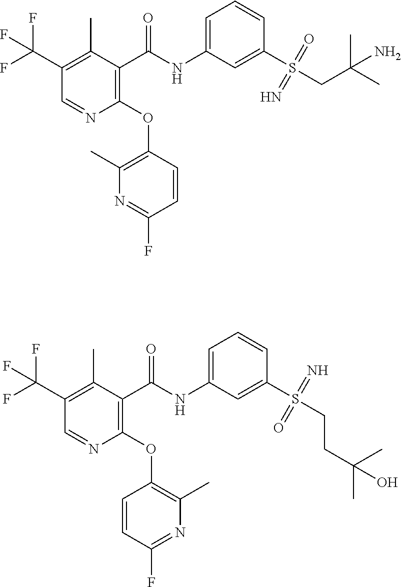 [SMILES: Cc1nc(F)ccc1Oc1ncc(C(F)(F)F)c(C)c1C(=O)Nc1cccc(S(=N)(=O)CC(C)(C)N)c1 ; Cc1nc(F)ccc1Oc1ncc(C(F)(F)F)c(C)c1C(=O)Nc1cccc(S(=N)(=O)CCC(C)(C)O)c1]

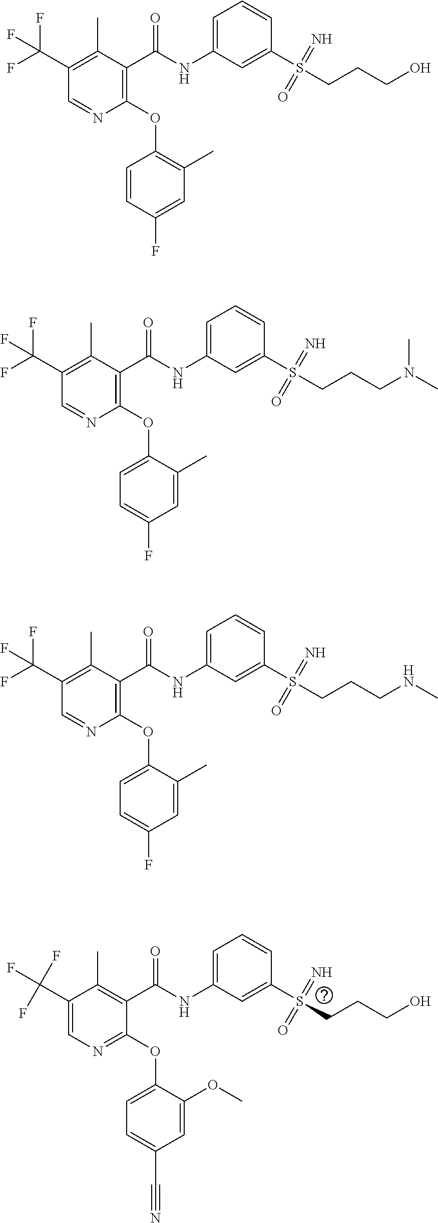 [SMILES: Cc1cc(F)ccc1Oc1ncc(C(F)(F)F)c(C)c1C(=O)Nc1cccc(S(=N)(=O)CCCO)c1 ; Cc1cc(F)ccc1Oc1ncc(C(F)(F)F)c(C)c1C(=O)Nc1cccc(S(=N)(=O)CCCN(C)C)c1 ; CNCCCS(=N)(=O)c1cccc(NC(=O)c2c(Oc3ccc(F)cc3C)ncc(C(F)(F)F)c2C)c1 ; COc1cc(C#N)ccc1Oc1ncc(C(F)(F)F)c(C)c1C(=O)Nc1cccc([S@](=N)(=O)CCCO)c1]

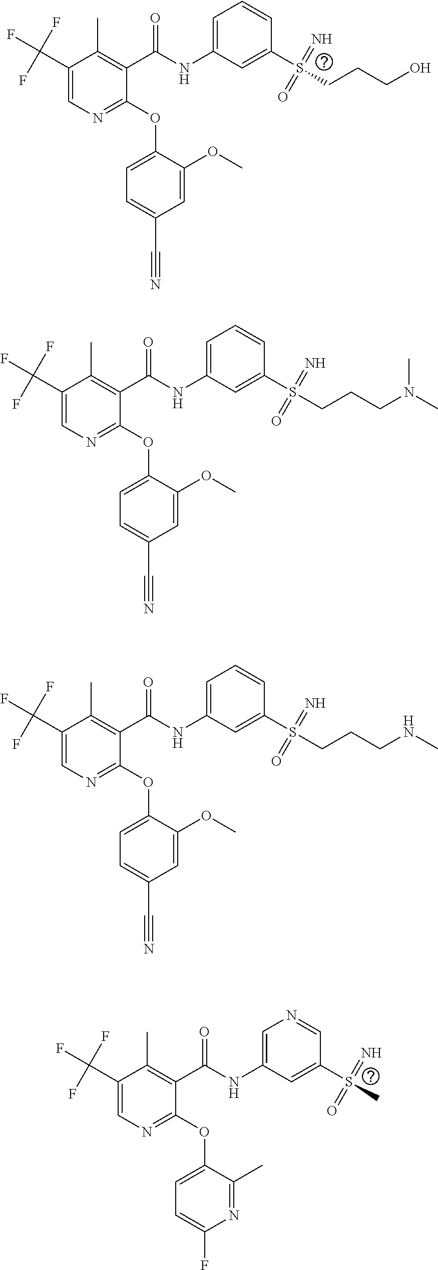 [SMILES: COc1cc(C#N)ccc1Oc1ncc(C(F)(F)F)c(C)c1C(=O)Nc1cccc([S@@](=N)(=O)CCCO)c1 ; COc1cc(C#N)ccc1Oc1ncc(C(F)(F)F)c(C)c1C(=O)Nc1cccc(S(=N)(=O)CCCN(C)C)c1 ; CNCCCS(=N)(=O)c1cccc(NC(=O)c2c(Oc3ccc(C#N)cc3OC)ncc(C(F)(F)F)c2C)c1 ; Cc1nc(F)ccc1Oc1ncc(C(F)(F)F)c(C)c1C(=O)Nc1cncc([S@](C)(=N)=O)c1]

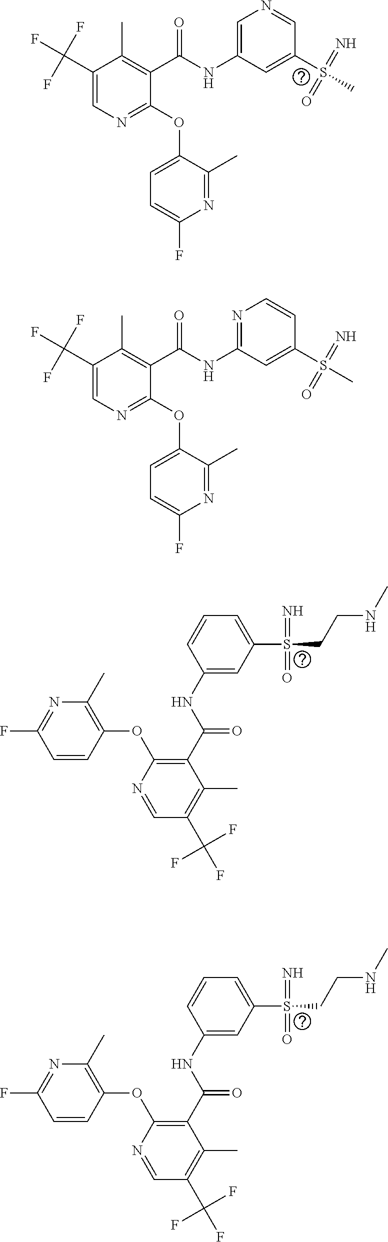 [SMILES: Cc1nc(F)ccc1Oc1ncc(C(F)(F)F)c(C)c1C(=O)Nc1cncc([S@@](C)(=N)=O)c1 ; Cc1nc(F)ccc1Oc1ncc(C(F)(F)F)c(C)c1C(=O)Nc1cc(S(C)(=N)=O)ccn1 ; CNCC[S@@](=N)(=O)c1cccc(NC(=O)c2c(Oc3ccc(F)nc3C)ncc(C(F)(F)F)c2C)c1 ; CNCC[S@](=N)(=O)c1cccc(NC(=O)c2c(Oc3ccc(F)nc3C)ncc(C(F)(F)F)c2C)c1]

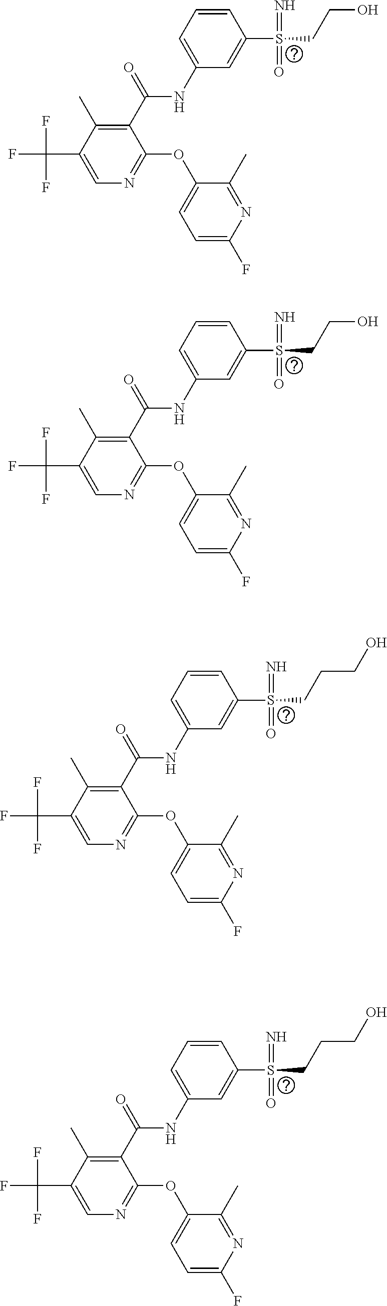 [SMILES: Cc1nc(F)ccc1Oc1ncc(C(F)(F)F)c(C)c1C(=O)Nc1cccc([S@@](=N)(=O)CCO)c1 ; Cc1nc(F)ccc1Oc1ncc(C(F)(F)F)c(C)c1C(=O)Nc1cccc([S@](=N)(=O)CCO)c1 ; Cc1nc(F)ccc1Oc1ncc(C(F)(F)F)c(C)c1C(=O)Nc1cccc([S@@](=N)(=O)CCCO)c1 ; Cc1nc(F)ccc1Oc1ncc(C(F)(F)F)c(C)c1C(=O)Nc1cccc([S@](=N)(=O)CCCO)c1]

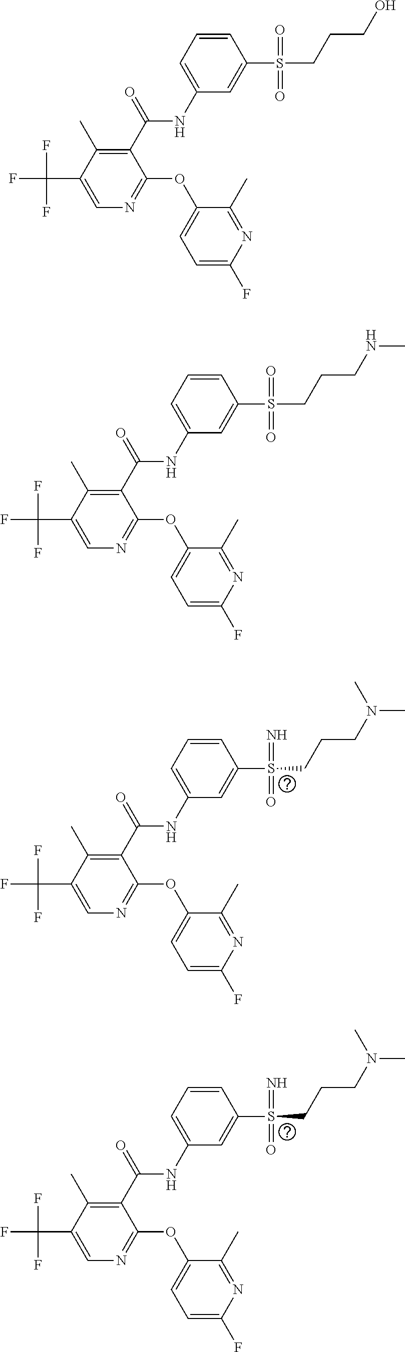 [SMILES: Cc1nc(F)ccc1Oc1ncc(C(F)(F)F)c(C)c1C(=O)Nc1cccc(S(=O)(=O)CCCO)c1 ; CNCCCS(=O)(=O)c1cccc(NC(=O)c2c(Oc3ccc(F)nc3C)ncc(C(F)(F)F)c2C)c1 ; Cc1nc(F)ccc1Oc1ncc(C(F)(F)F)c(C)c1C(=O)Nc1cccc([S@@](=N)(=O)CCCN(C)C)c1 ; Cc1nc(F)ccc1Oc1ncc(C(F)(F)F)c(C)c1C(=O)Nc1cccc([S@](=N)(=O)CCCN(C)C)c1]

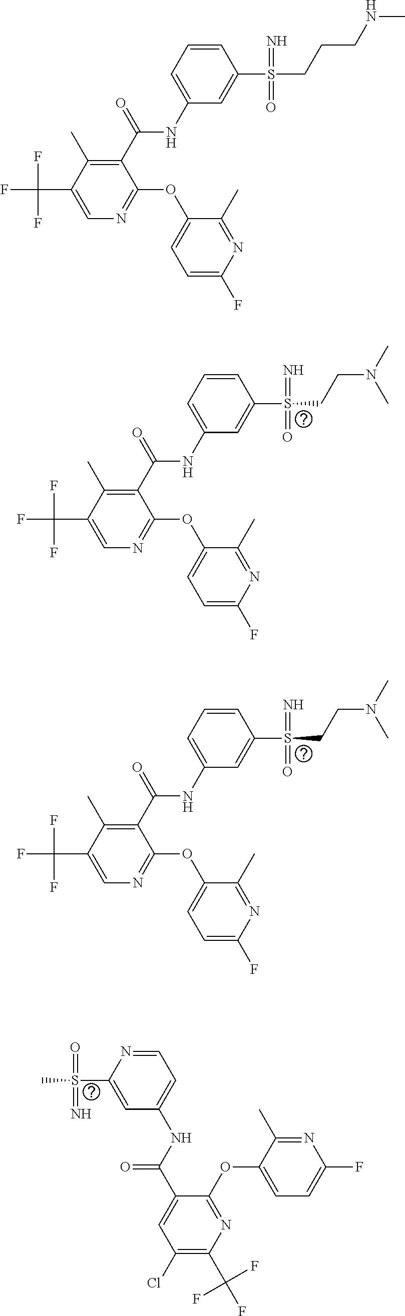

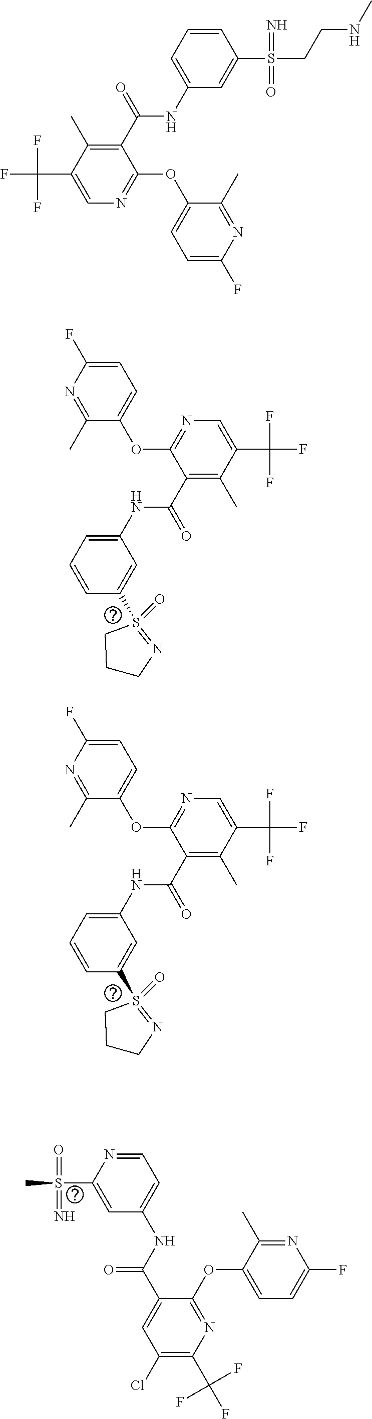

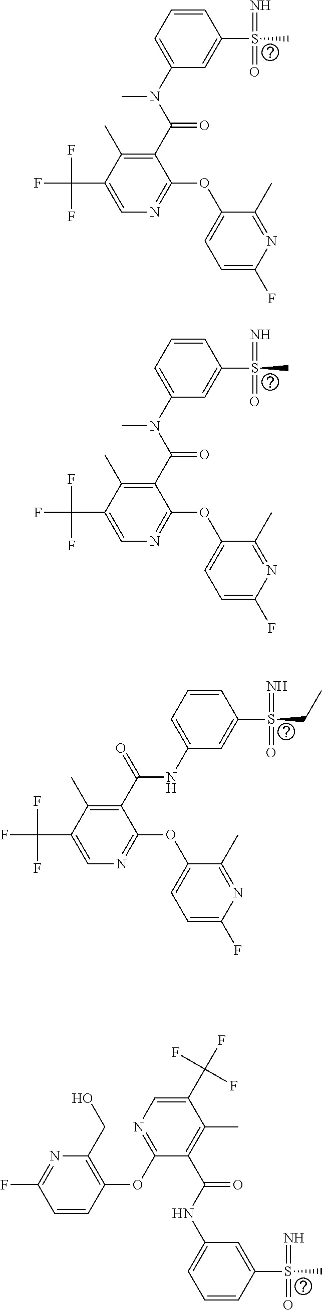

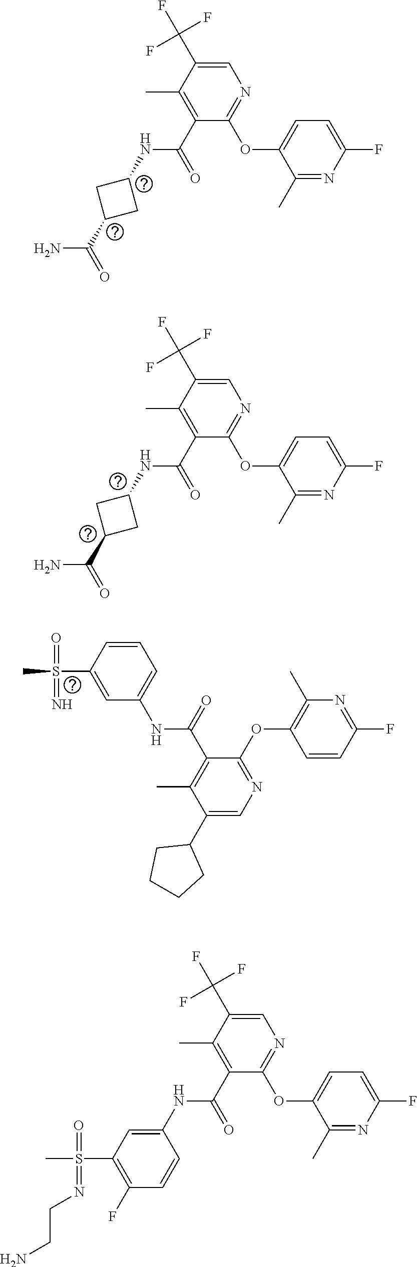 [SMILES: Cc1nc(F)ccc1Oc1ncc(C(F)(F)F)c(C)c1C(=O)N[C@H]1C[C@@H](C(N)=O)C1 ; Cc1nc(F)ccc1Oc1ncc(C(F)(F)F)c(C)c1C(=O)N[C@H]1C[C@H](C(N)=O)C1 ; Cc1nc(F)ccc1Oc1ncc(C2CCCC2)c(C)c1C(=O)Nc1cccc([S@](C)(=N)=O)c1 ; Cc1nc(F)ccc1Oc1ncc(C(F)(F)F)c(C)c1C(=O)Nc1ccc(F)c(S(C)(=O)=NCCN)c1]

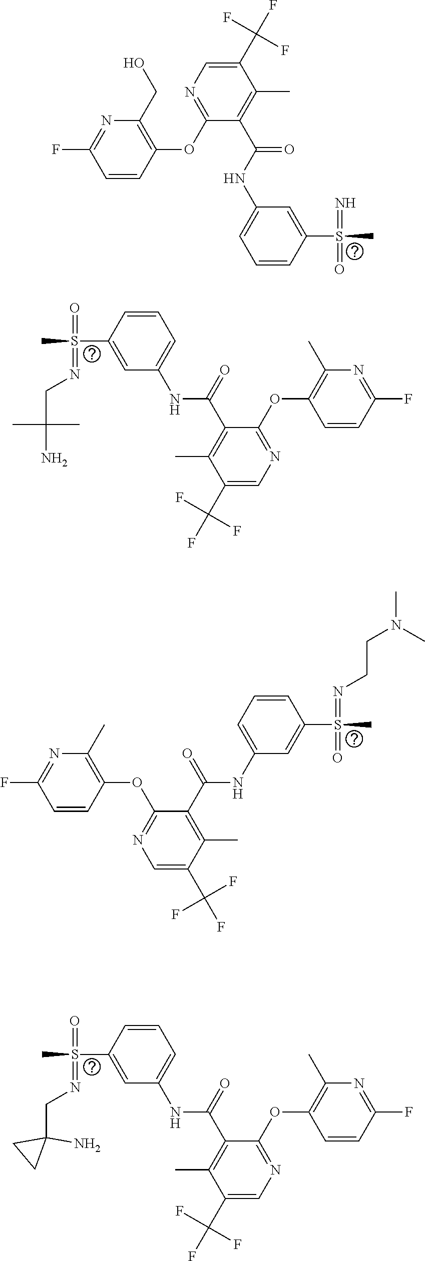

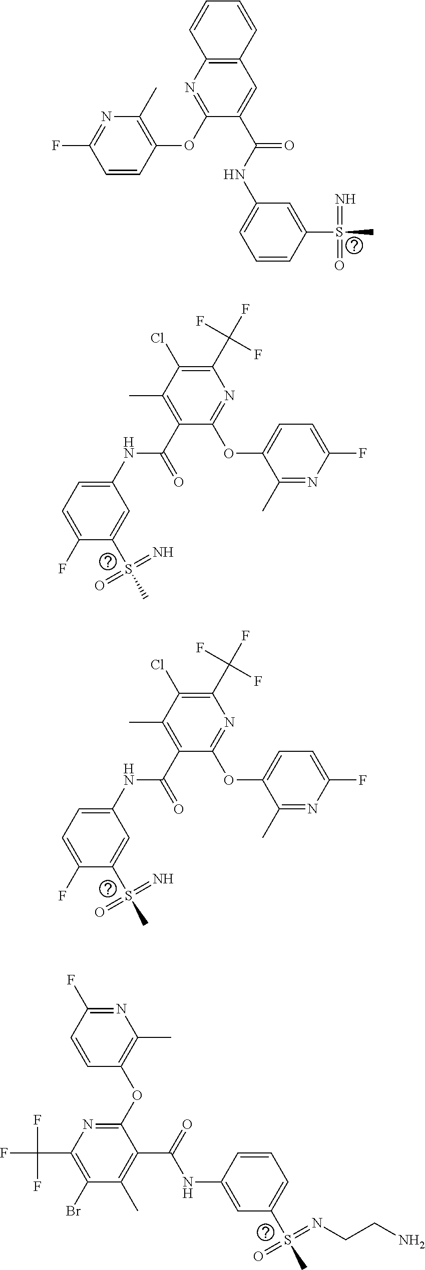 [SMILES: Cc1nc(F)ccc1Oc1nc2ccccc2cc1C(=O)Nc1cccc([S@](C)(=N)=O)c1 ; Cc1nc(F)ccc1Oc1nc(C(F)(F)F)c(Cl)c(C)c1C(=O)Nc1ccc(F)c([S@@](C)(=N)=O)c1 ; Cc1nc(F)ccc1Oc1nc(C(F)(F)F)c(Cl)c(C)c1C(=O)Nc1ccc(F)c([S@](C)(=N)=O)c1 ; Cc1nc(F)ccc1Oc1nc(C(F)(F)F)c(Br)c(C)c1C(=O)Nc1cccc([S@@](C)(=O)=NCCN)c1]

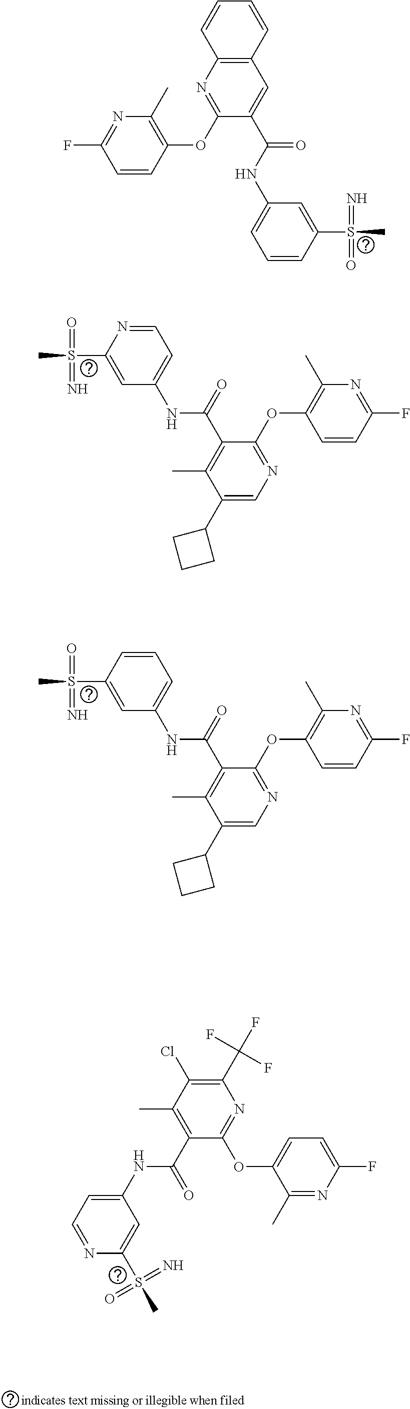

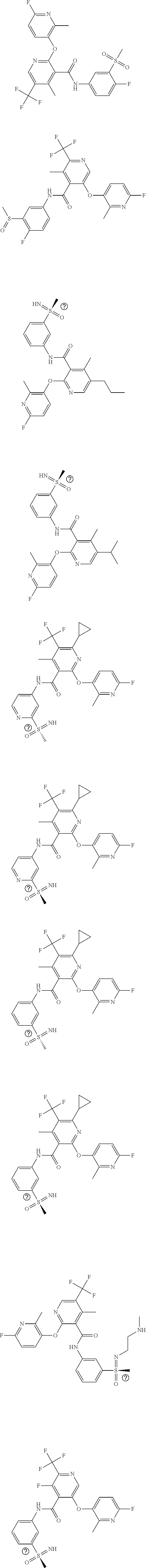

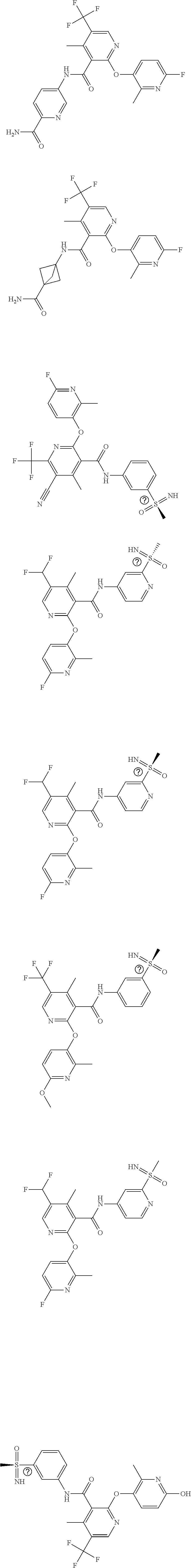

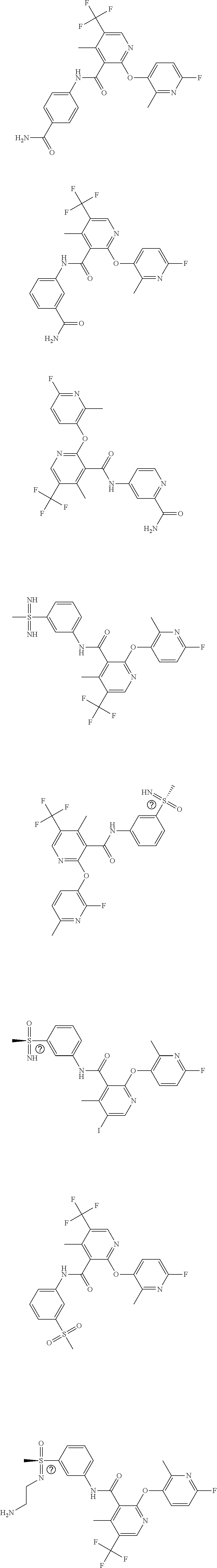

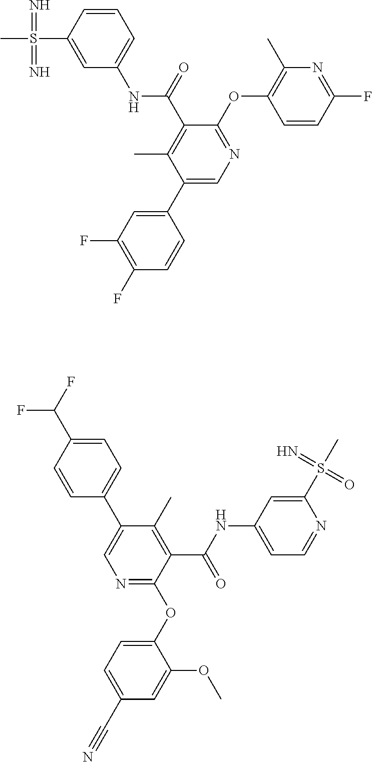 [SMILES: Cc1nc(F)ccc1Oc1ncc(-c2ccc(F)c(F)c2)c(C)c1C(=O)Nc1cccc(S(C)(=N)=N)c1 ; COc1cc(C#N)ccc1Oc1ncc(-c2ccc(C(F)F)cc2)c(C)c1C(=O)Nc1ccnc(S(C)(=N)=O)c1]

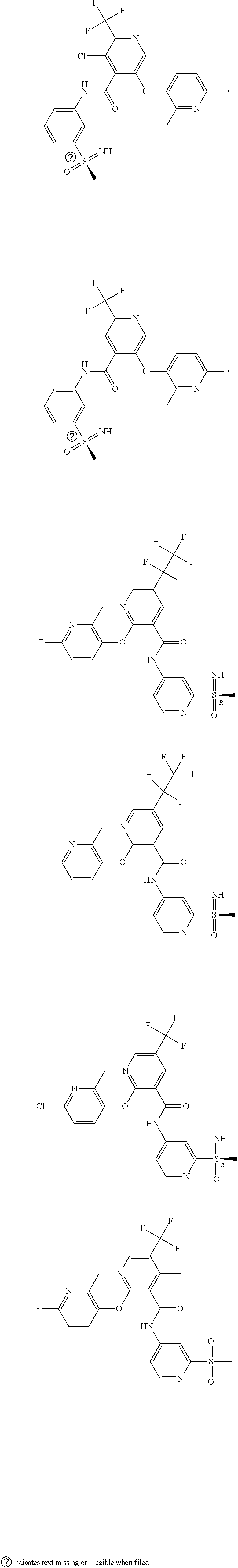

29. The compound of claim 1, wherein R₂ is Formula (II): 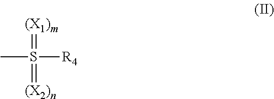
wherein,
m and n are independently 0 or 1; and
X₁ and X₂ are not both O, NH, or NR′.

30. A method of treating a condition in a subject, the method comprising providing to a subject having a condition, a compound selected from the compounds recited in claims 1-29.

31. A compound of Formula (I): 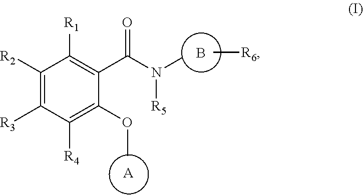
and pharmaceutically acceptable salts, hydrates, and solvates thereof,
wherein, A is aryl or heteroaryl wherein the aryl or heteroaryl is unsubstituted or substituted with one or more groups selected from the group consisting of halo-C₁-C₄ alkyl wherein the haloalkyl chain may be fully or partially halogenated, substituted or unsubstituted C₁-C₈ alkyl, deuterated C₁-C₄ alkyl wherein the alkyl chain may be fully or partially deuterated, C₃-C₁₀ cycloalkyl, halogen, cyano, nitro, C₁-C₈ alkoxyl, haloalkoxyl, wherein the haloalkoxy chain may be fully or partially halogenated, or arylalkoxyl;
B is aryl or heteroaryl wherein the aryl may have 1 to 4 substituents and heteroaryl may have 1 to 3 substituents, these substituents are independently selected from halogen, C₁-C₈ alkyl, haloalkyl, or alkoxy;
R₁, R₂, R₃ and R₄ are independently selected from H, halogen, —OH, C₁-C₆-alkyl, C₁-C₆ fluoroalkyl wherein the fluoroalkyl chain may be fully or partially fluorinated, C₃-C₈ branched alkyl, C₃-C₈ branched fluoroalkyl wherein the branched fluoroalkyl chain may be fully or partially fluorinated, alkenyl, alkynyl, haloalkyl, alkoxy, cycloalkoxy, haloalkoxy, thioalkoxy, nitro, cyano, —C(R′)(R″)-cycloalkyl, C(R′)(R′)-aryl, —NR′R″, substituted or unsubstituted 3-8 membered cycloalkyl, 3-8 membered cycloalkenyl, or 3-6 membered heterocycloalkyl, wherein the 3-6 membered heterocycloalkyl comprises at least one heteroatom independently selected from O, S, and N;
R₈ is H or C₁-C₃ alkyl; and
R₆—C(═O)NH₂, —C(═O)NHR′, —C(═O)NR′R″, or Formula (II): 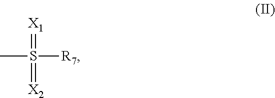
wherein:
X₁ and X₂ are both O, NH, or NR′; or
X₁ is O and X₂ is either NH or NR′;
R₇ is NH₂, NHR′, NR′R″, C₁-C₃ alkyl, C₃-C₈ cycloalkyl, haloalkyl, halocycloalkyl, aryl, heteroaryl, or heterocyclyl.

32. The compound of claim 31, wherein the compound is a compound of Formula (III): 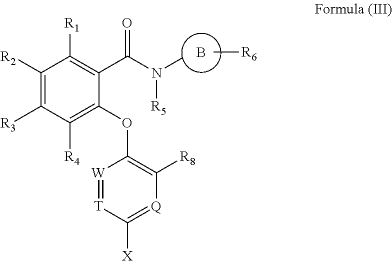
wherein, R₁, R₂, R₃, R₄, R₅, R₆, and B are described in claim 1;
Q, T and W is independently N or CR₉;
R₉ is H, halogen, —CD₃, alkyl, haloalkyl, alkoxy, haloalkoxy, cyano, —CF₃, —OCF₃, or substituted or unsubstituted cycloalkoxy;
X is H, halogen, —CD₃, alkyl, haloalkyl, alkoxy, cycloalkoxy, haloalkoxy, cyano, —CF₃, — or OCF₃;
R₈ is H, hydroxyl, halogen, —CD₃, C₁-C₆-alkyl, branched alkyl, haloalkyl where the alkyl chain is fully or partially halogenated, alkoxy, arylalkoxy, cycloalkoxy, haloalkoxy, cyano, —CH₂-cycloalkyl, —CH(CH₃)-cycloalkyl, trifluoromethyl, cyclopropylmethyl, or substituted or unsubstituted 3-6 membered cycloalkyl.

33. The compound of claim 31, wherein B is: 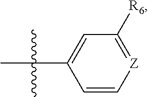
wherein R₆ is described above; Z is CR₁₀, N, or N⁻O⁻; wherein R₁₀ is H, halo, —CD₃, C₁-C₈ alkyl, haloalkyl, or alkoxy.

34. The compound of claim 33, wherein R₆ is 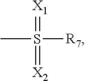
wherein:
X₁ and X₂ are both O, NH, or NR′; or
X₁ is O and X₂ is either NH or NR′;
R₇ is NH₂, NHR′, NR′R″, C₁-C₃ alkyl, C₃-C₈ cycloalkyl, haloalkyl, halocycloalkyl, aryl, heteroaryl, or heterocyclyl.

35. The compound of claim 33, wherein R₆ is 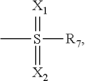
wherein:
X₁ is O, X₂ is NH, and R₇ is C₁-C₃ alkyl.

36. The compound of claim 31, wherein R₆ does not include —C(═O)NH₂, —C(═O)NHR′, or —C(═O)NR′R″.

37. The compound of claim 31, wherein B is a phenyl ring.

38. The compound of claim 31, wherein B is a pyridine ring.

39. The compound of claim 31, wherein R₁ is H, —CH₃, or F.

40. The compound of claim 31, wherein R₂ is chloro, —CF₃, H, 2-pyrazoline, or 1-methyl-1H-pyrazol-4-yl.

41. The compound of claim 31, wherein R₃ is H, —CF₃, or F.

42. The compound of claim 31, wherein R₄ is H.

43. The compound of claim 32, wherein R₈ is H, —CH₃, or —O—CH₃.

44. The compound of claim 32, wherein R₇ is —CH₃.

45. The compound of claim 32, wherein X is F or —CN.

46. The compound of claim 32, wherein Q is N or CH.

47. The compound of claim 32, wherein W is CH and T is CH.

48. The compound of claim 33, wherein Z is CR₁₀, and R₁₀ is H or F.

49. The compound of claim 31, wherein the compound is selected from a group consisting of:

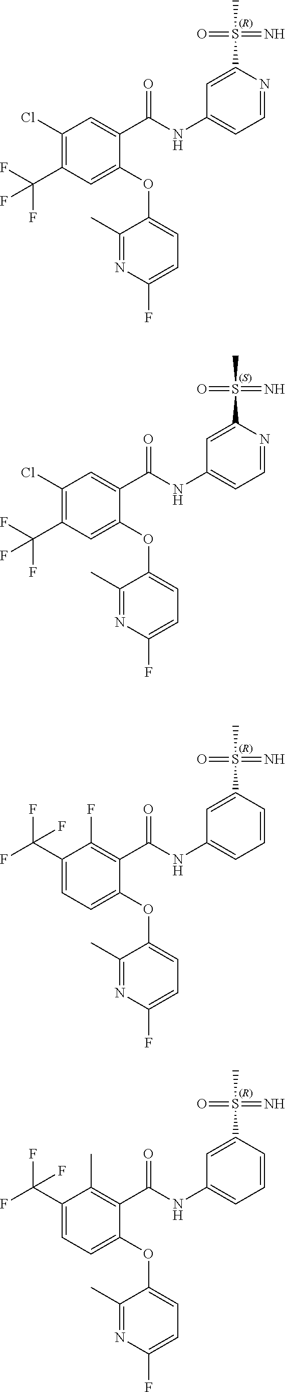

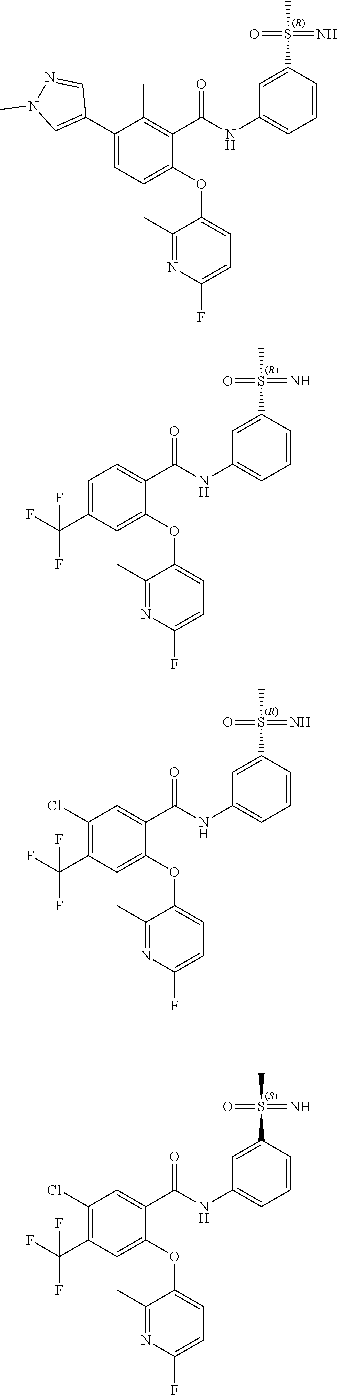

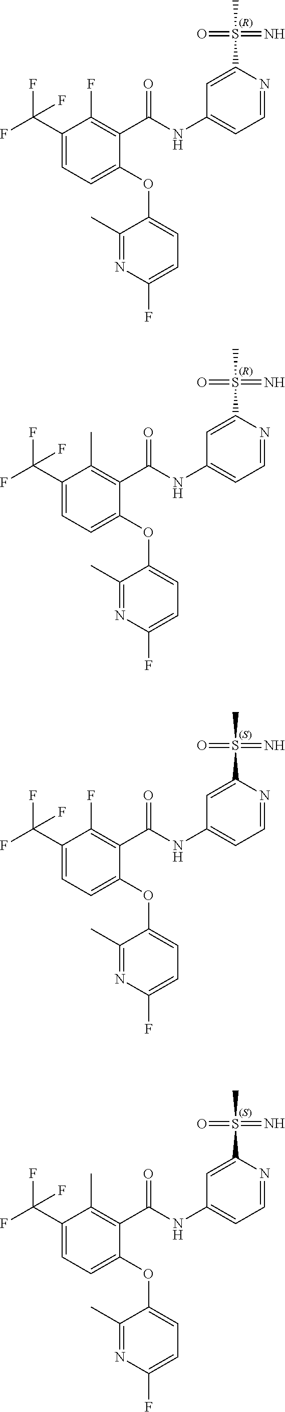

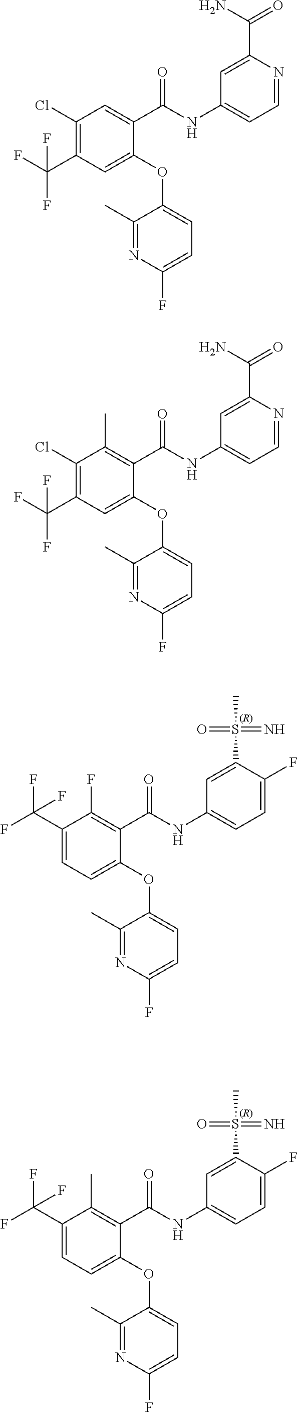

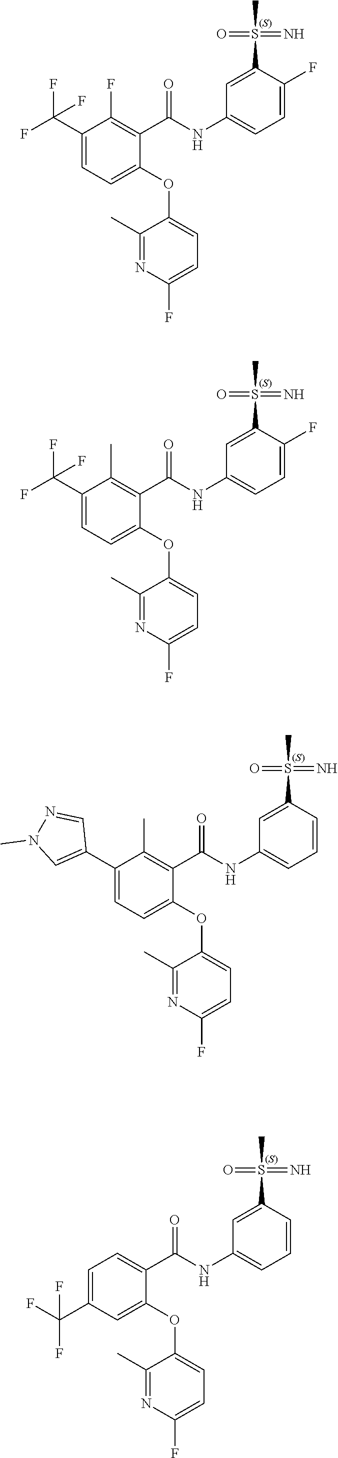

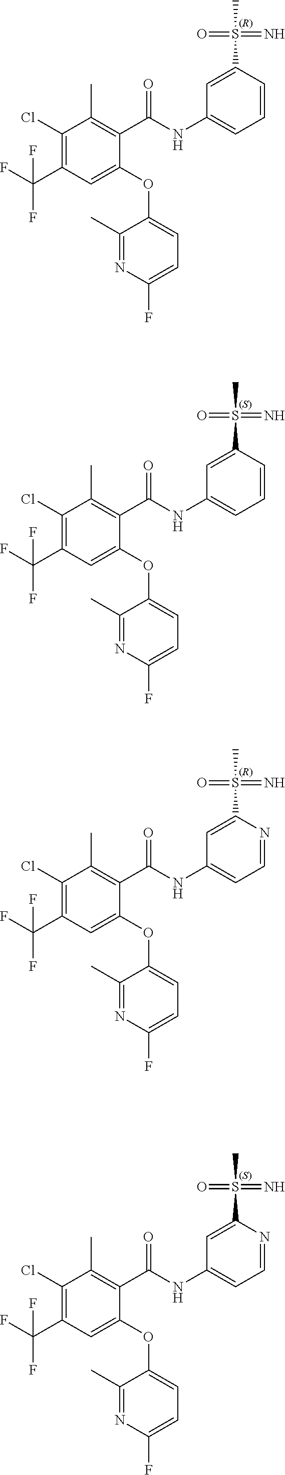

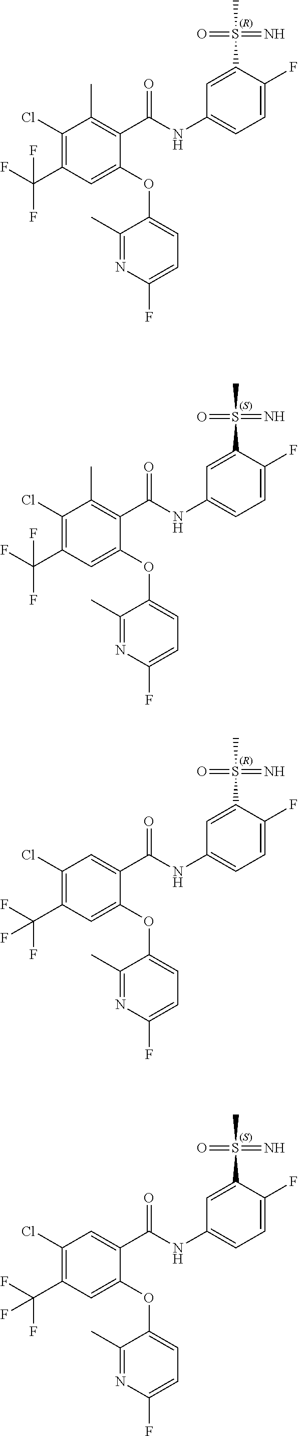

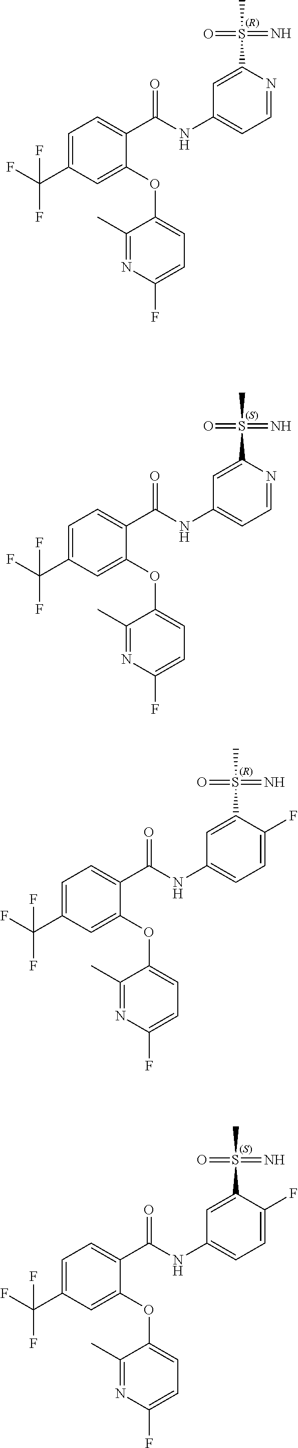

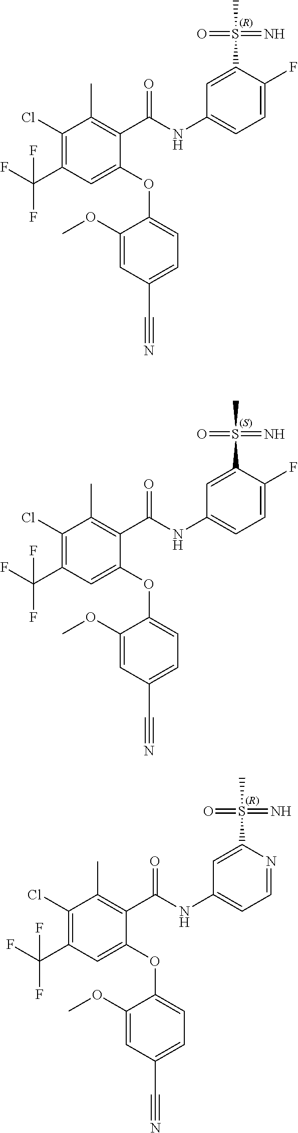

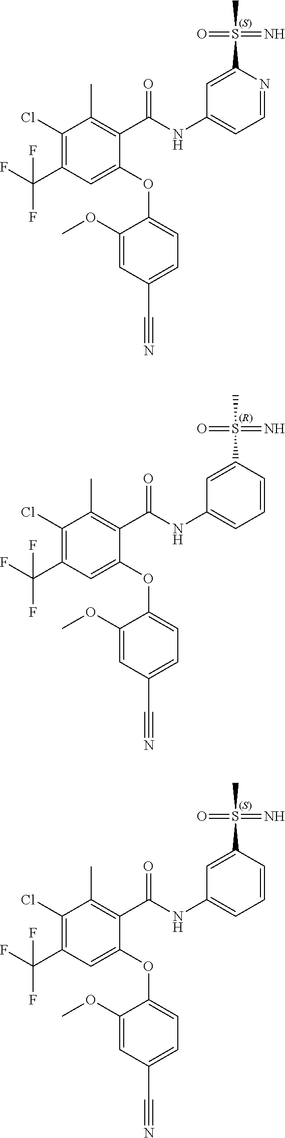

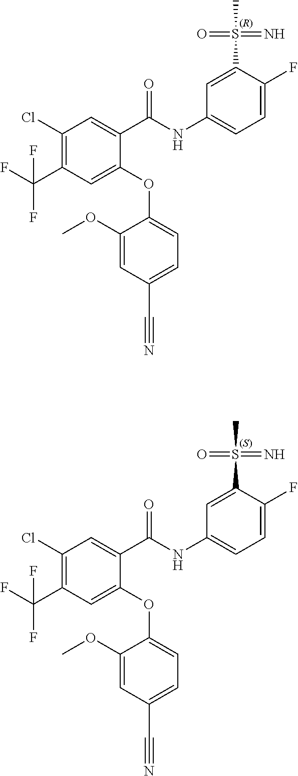

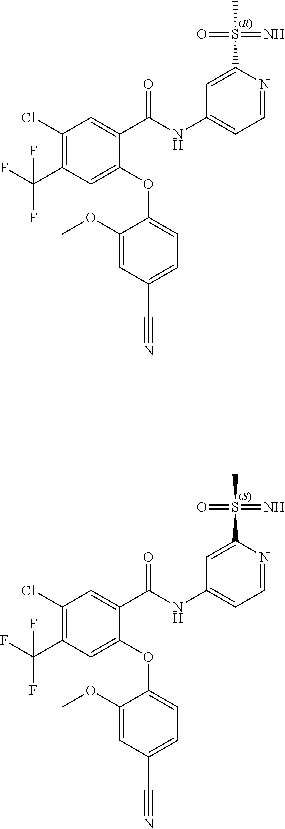

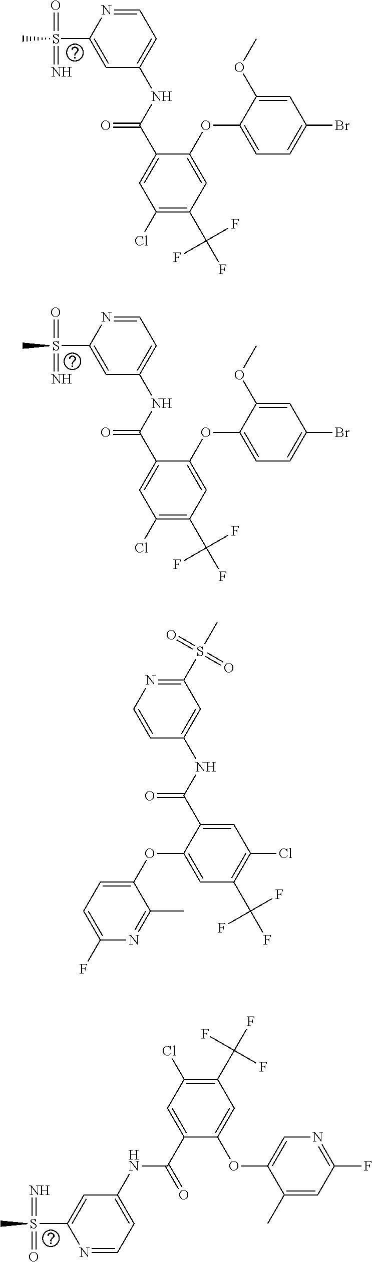

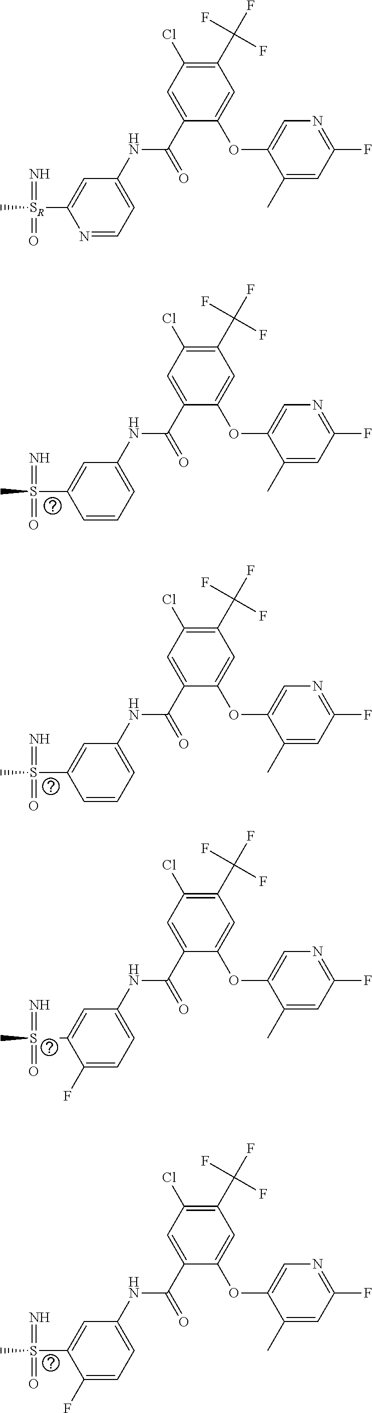

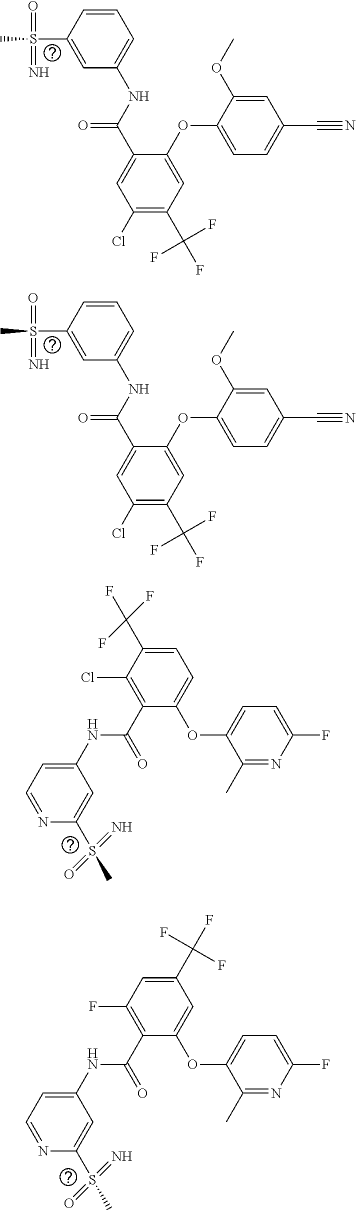

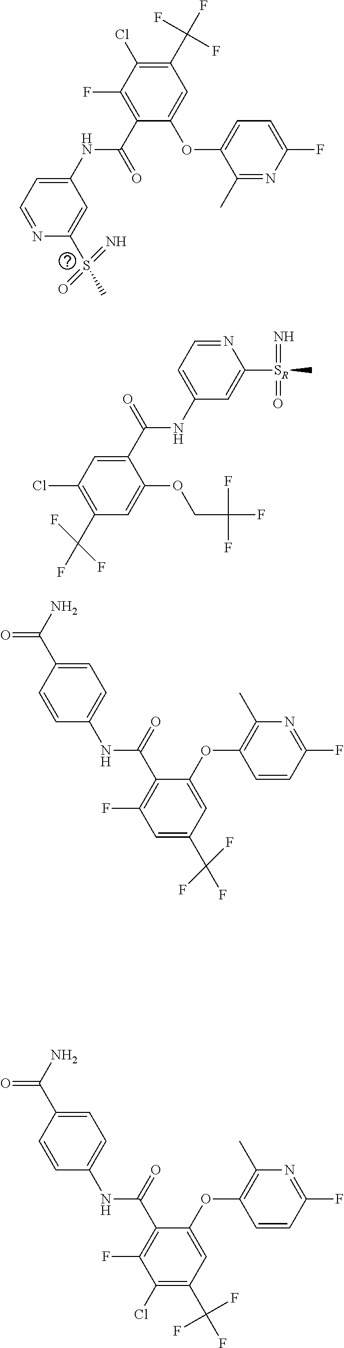

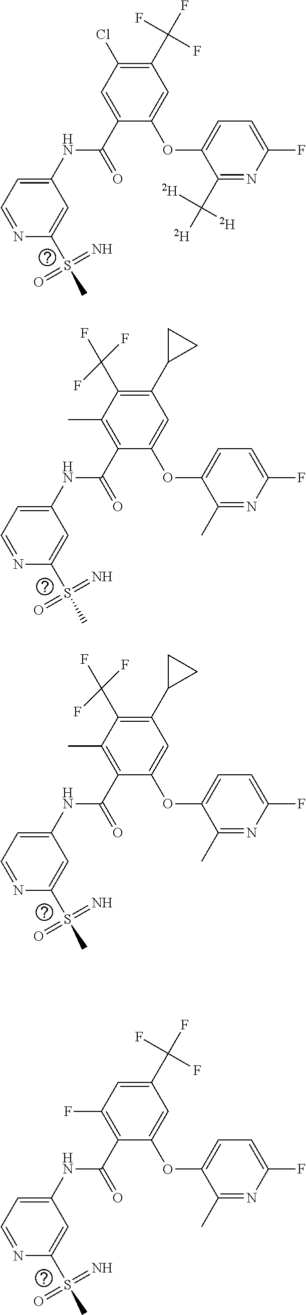

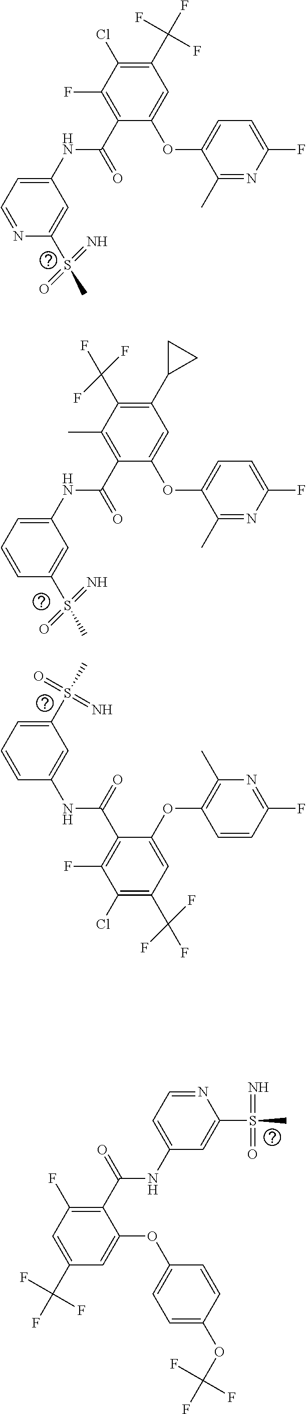

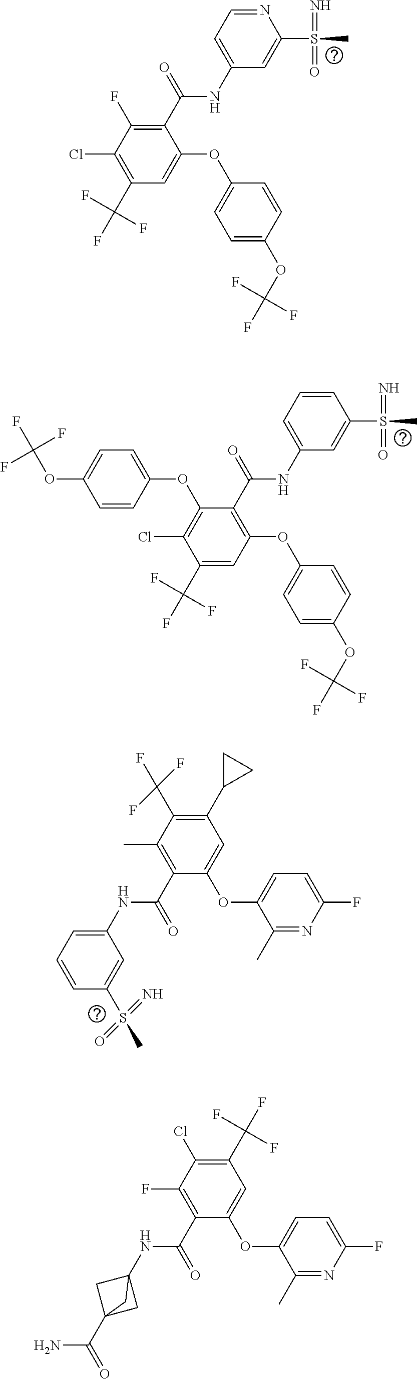 [SMILES: C[S@@](=N)(=O)c1cc(NC(=O)c2c(Oc3ccc(OC(F)(F)F)cc3)cc(C(F)(F)F)c(Cl)c2F)ccn1 ; C[S@@](=N)(=O)c1cccc(NC(=O)c2c(Oc3ccc(OC(F)(F)F)cc3)cc(C(F)(F)F)c(Cl)c2Oc2ccc(OC(F)(F)F)cc2)c1 ; Cc1nc(F)ccc1Oc1cc(C2CC2)c(C(F)(F)F)c(C)c1C(=O)Nc1cccc([S@](C)(=N)=O)c1 ; Cc1nc(F)ccc1Oc1cc(C(F)(F)F)c(Cl)c(F)c1C(=O)NC12CC(C(N)=O)(C1)C2]

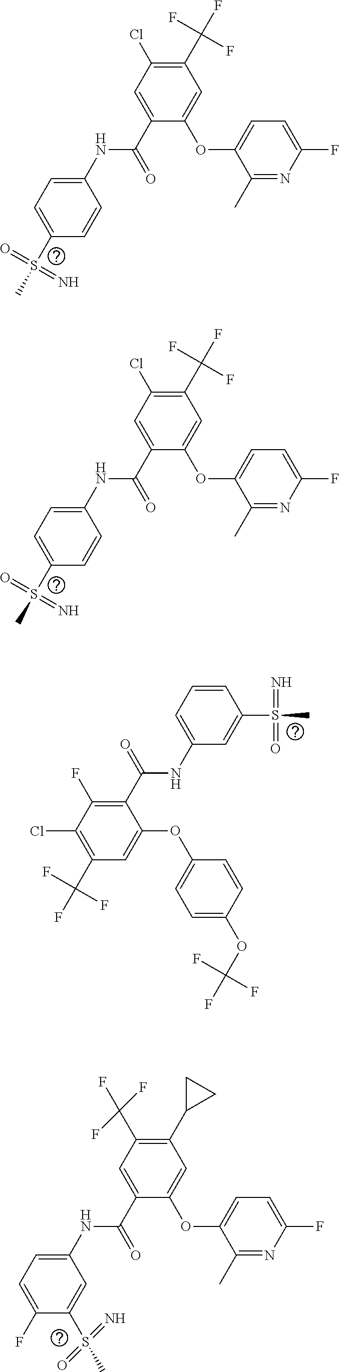 [SMILES: Cc1nc(F)ccc1Oc1cc(C(F)(F)F)c(Cl)cc1C(=O)Nc1ccc([S@@](C)(=N)=O)cc1 ; Cc1nc(F)ccc1Oc1cc(C(F)(F)F)c(Cl)cc1C(=O)Nc1ccc([S@](C)(=N)=O)cc1 ; C[S@@](=N)(=O)c1cccc(NC(=O)c2c(Oc3ccc(OC(F)(F)F)cc3)cc(C(F)(F)F)c(Cl)c2F)c1 ; Cc1nc(F)ccc1Oc1cc(C2CC2)c(C(F)(F)F)cc1C(=O)Nc1ccc(F)c([S@@](C)(=N)=O)c1]

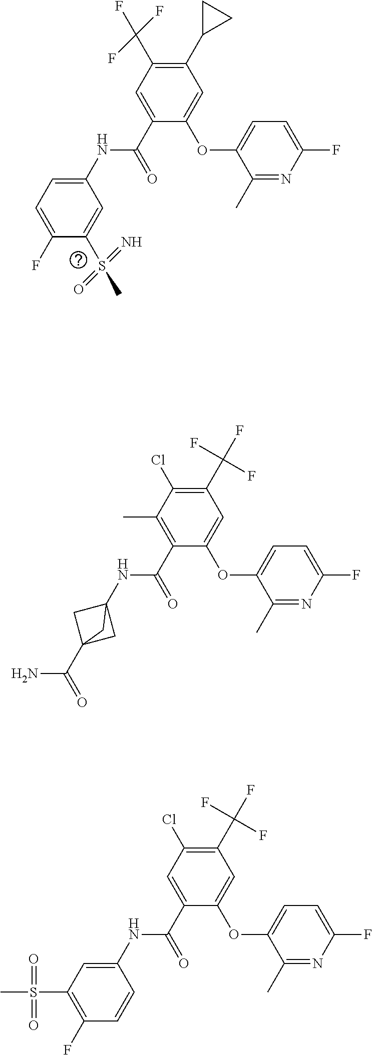 [SMILES: Cc1nc(F)ccc1Oc1cc(C2CC2)c(C(F)(F)F)cc1C(=O)Nc1ccc(F)c([S@](C)(=N)=O)c1 ; Cc1nc(F)ccc1Oc1cc(C(F)(F)F)c(Cl)c(C)c1C(=O)NC12CC(C(N)=O)(C1)C2 ; Cc1nc(F)ccc1Oc1cc(C(F)(F)F)c(Cl)cc1C(=O)Nc1ccc(F)c(S(C)(=O)=O)c1]

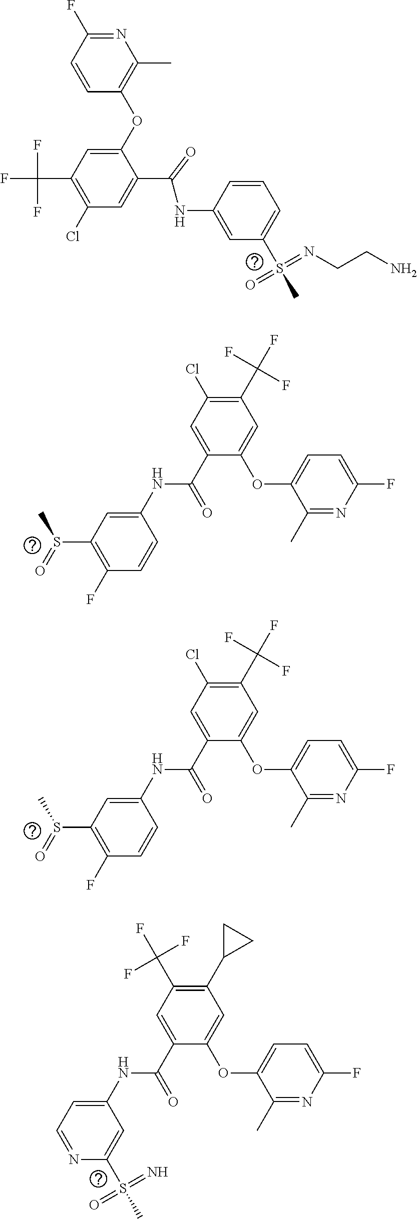 [SMILES: Cc1nc(F)ccc1Oc1cc(C(F)(F)F)c(Cl)cc1C(=O)Nc1cccc([S@@](C)(=O)=NCCN)c1 ; Cc1nc(F)ccc1Oc1cc(C(F)(F)F)c(Cl)cc1C(=O)Nc1ccc(F)c([S@](C)=O)c1 ; Cc1nc(F)ccc1Oc1cc(C(F)(F)F)c(Cl)cc1C(=O)Nc1ccc(F)c([S@@](C)=O)c1 ; Cc1nc(F)ccc1Oc1cc(C2CC2)c(C(F)(F)F)cc1C(=O)Nc1ccnc([S@@](C)(=N)=O)c1]

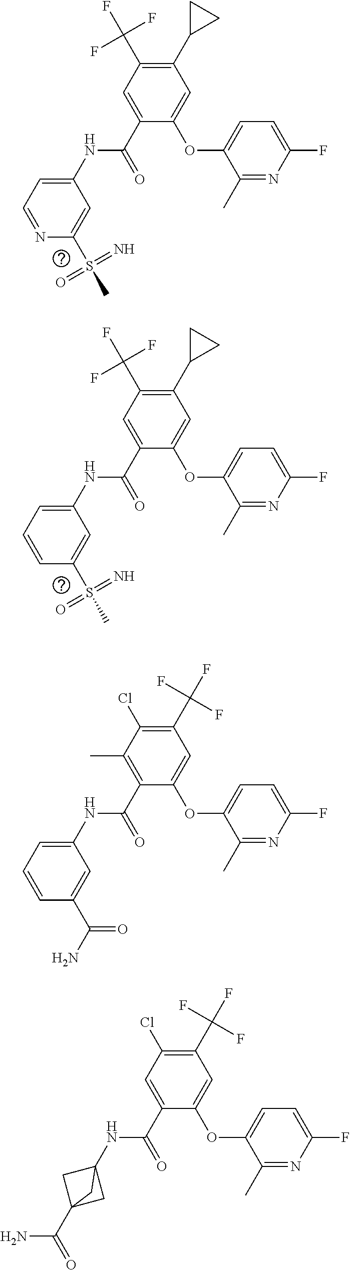

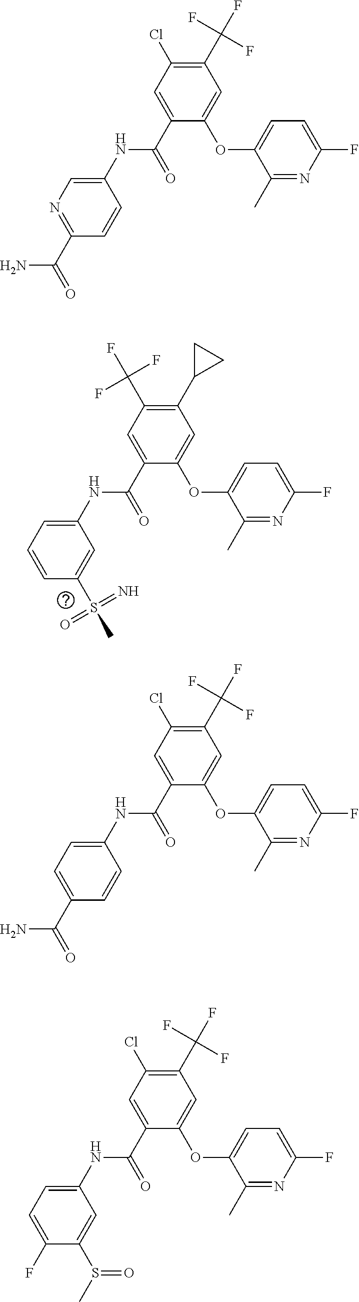 [SMILES: Cc1nc(F)ccc1Oc1cc(C(F)(F)F)c(Cl)cc1C(=O)Nc1ccc(C(N)=O)nc1 ; Cc1nc(F)ccc1Oc1cc(C2CC2)c(C(F)(F)F)cc1C(=O)Nc1cccc([S@](C)(=N)=O)c1 ; Cc1nc(F)ccc1Oc1cc(C(F)(F)F)c(Cl)cc1C(=O)Nc1ccc(C(N)=O)cc1 ; Cc1nc(F)ccc1Oc1cc(C(F)(F)F)c(Cl)cc1C(=O)Nc1ccc(F)c(S(C)=O)c1]

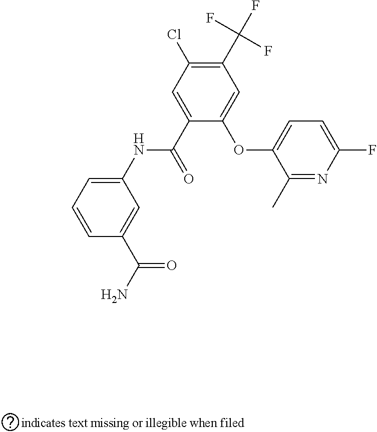 [SMILES: Cc1nc(F)ccc1Oc1cc(C(F)(F)F)c(Cl)cc1C(=O)Nc1cccc(C(N)=O)c1]

50. A method of treating a condition in a subject, the method comprising providing to a subject having a condition, a compound selected from the compounds recited in claims 31-49.

51. A compound of Formula (I): 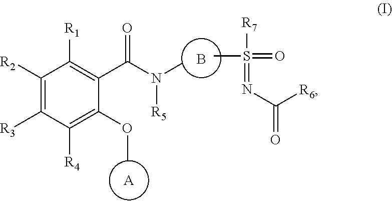
and pharmaceutically acceptable salts, hydrates, and solvates thereof, wherein:
A and B are independently aryl or heteroaryl, wherein the aryl or heteroaryl is unsubstituted or substituted with one or more groups selected from the group consisting of halo-C₁-C₄ alkyl wherein the haloalkyl chain may be fully or partially halogenated, substituted or unsubstituted C₁-C₈ alkyl, deuterated C₁-C₄ alkyl wherein the alkyl chain may be fully or partially deuterated, C₃-C₁₀ cycloalkyl, halogen, cyano, nitro, C₁-C₈ alkoxyl, aloalkoxyl wherein the haloalkyoxy chain may be fully or partially halogenated, and arylalkoxyl;
R₁, R₂, R₃ and R₄ are independently selected from hydrogen, —OH, halogen, C₁-C₆-alkyl, C₁-C₆ fluoroalkyl wherein the fluoroalkyl chain may be fully or partially fluorinated, C₃-C₅ branched alkyl, C₃-C₈ branched fluoroalkyl wherein the branched fluoroalkyl chain may be fully or partially fluorinated, alkenyl, alkynyl, haloalkyl, alkoxy, cycloalkoxy, haloalkoxy, thioalkoxy, nitro, cyano, —C(R′)(R′)-cycloalkyl, C(R′)(R′)-aryl, NR′R″, 3-8, membered cycloalkyl, 3-8-membered cycloalkenyl 3-6 membered heterocycloalkyl, any of which may have one or more substituents, wherein the 3-6 membered heterocycloalkyl comprises at least one heteroatom independently selected from O, S, and N, a saturated or unsaturated 5 or more membered ring or an aryl ring optionally containing 1 or more heteroatoms independently selected from 0, S, and N, wherein each 5 or more membered ring is unsubstituted or substituted with one to five substituents selected from hydrogen, cyano, halo, or methyl; a fused ring formed by at least two of R₁, R₂, R₃ and R₄ wherein the fused ring is selected from a group consisting of: optionally saturated carbocyclyl or heterocyclyl containing 5-6 ring members, wherein the heterocyclyl include one or more heteroatoms;
R₅ is H, or substituted or unsubstituted C₁-C₃ alkyl;
R₆ is —(CH₂)ₙRₐ, —(CR_{b}R_{c})ₙRₐ, —(CR_{b}R_{c})ₙ—(OCH₂CH₂)ₙRₐ, —(CR_{b}R_{c})ₙ—(NR′CH₂CH₂)ₙRₐ, —(CR_{b}R_{c})ₙ—(NR′CH₂CH₂O)ₙRₐ, or —(CR_{b}R_{c})ₙ—(NHCH₂CH₂NH)ₙRₐ;
wherein Rₐ is H, C₁-C₆-alkyl, C₂-C₈ branched alkyl, C₃-C₈ cycloalkyl, aryl, heteroaryl, 4-7 member heterocyclyl, alkenyl, alkynyl, haloalkyl, OH, alkoxy, cycloalkoxy, haloalkoxy, —(OCH₂CH₂)ₙR_{b}, —NR′R″, —COONR′R″, —COOR′, alkylsulfonyl, arylsulfonyl, or —SO₂NR′R″;
R_{b} is independently selected from H, F, C₁-C₆-alkyl, haloalkyl, branched alkyl, aryl, heteroaryl, 3-7 member carbocyclyl, 4-7 member heterocyclyl with one or more heteroatoms;
R_{c} is H, C₁-C₆ alkyl or F;
R_{c} and R_{b} optionally form a 3-6 member carbocyclic ring or 4-6 member heterocyclic ring with one or more hetero atoms;
R₇ is NH₂, —NHR′, C₁-C₃ alkyl, substituted or unsubstituted C₃-C₄ cycloalkyl, haloalkyl, halocycloalkyl, aryl, heteroaryl, or heterocyclyl.

52. The compound of claim 51, wherein the compound is a compound of Formula (II): 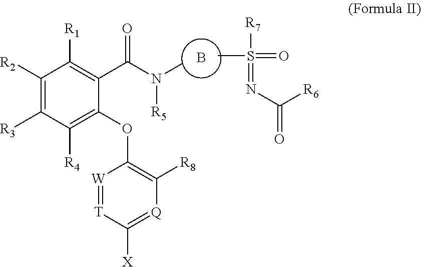
wherein ring B, and groups R₁, R₂, R₃, R₄, R₅, R₆, and R₇, are described in claim 1;
R₈ is H, hydroxyl, halogen, —CD₃, C₁-C₆-alkyl, branched alkyl, haloalkyl where the alkyl chain is fully or partially halogenated, alkoxy, arylalkoxy, cycloalkoxy, haloalkoxy, cyano, —CH₂-cycloalkyl, —CH(CH₃)-cycloalkyl, trifluoromethyl, cyclopropylmethyl, 3-6 membered cycloalkyl, or 3-6 membered heterocycloalkyl, any of which may have one or more substituents, wherein the 3-6 membered heterocycloalkyl comprises at least one heteroatom independently selected from O, S, and N;
Q, T, and W are independently selected from N or CR₉;
R₉ is H, halogen, —CD₃, alkyl, haloalkyl, alkoxy, haloalkoxy, cyano, —CF₃, —OCF₃, or cycloalkoxy, each of which is optionally substituted,
X is H, halogen, —CD₃, alkyl, haloalkyl, alkoxy, cycloalkoxy, haloalkoxy, cyano, —CF₃, or —OCF₃.

53. The compound of claim 51, wherein the compound is a compound of Formula (III): 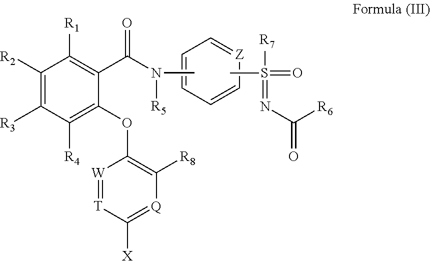
wherein, R₁, R₂, R₃, R₄, R₅, R₆, and R₇ are described in claim 1;
R₈, W, T, Q, and X are described in claim 52;
Z is CH, N, CF, or N⁺—O⁻

54. The compound of claim 52, wherein X is F.

55. The compound of claim 52, wherein R₈ is —CH₃.

56. The compound of claim 52, wherein Q is N.

57. The compound of claim 52, wherein Q is N, W is CH, and T is CH.

58. The compound of claim 51, wherein R₁ is H or F.

59. The compound of claim 51, wherein R₂ is H, chloro, or CF₃.

60. The compound of claim 51, wherein R₃ is H or CF₃.

61. The compound of claim 51, wherein R₄ is H.

62. The compound of claim 51, wherein R₅ is H.

63. The compound of claim 51, wherein R₇ is methyl.

64. The compound of claim 51, wherein R₆ is azetidine, pyrrolidine, —CH₂—OH, —CH—(CH₃)—OH, —CH—CH₂—NH—CH₃, —CH₂—NH₂, CH—(CH₃)—NH₂, —CH₂—NH₂, —C—(CH₃)₂—NH₂, or -cyclobutyl-NH₂.

65. The compound of claim 52, wherein R₉ is H.

66. The compound of claim 51, wherein the compound of Formula (I) is selected from the group consisting of:

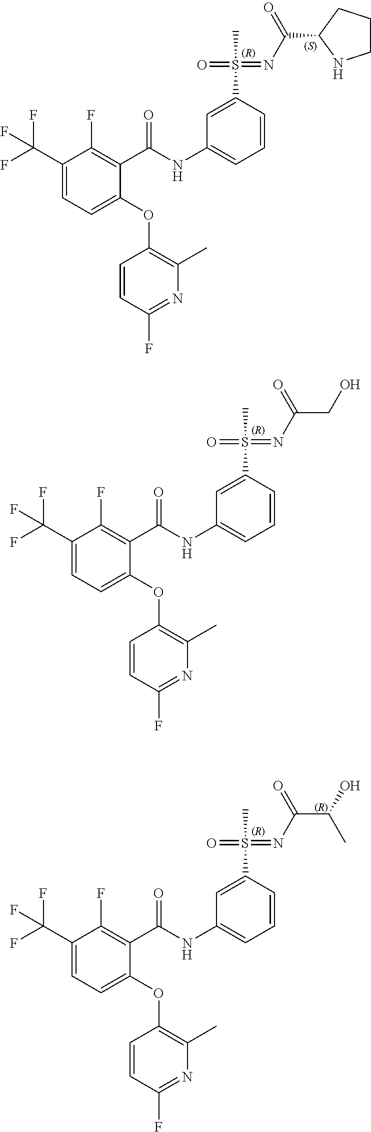

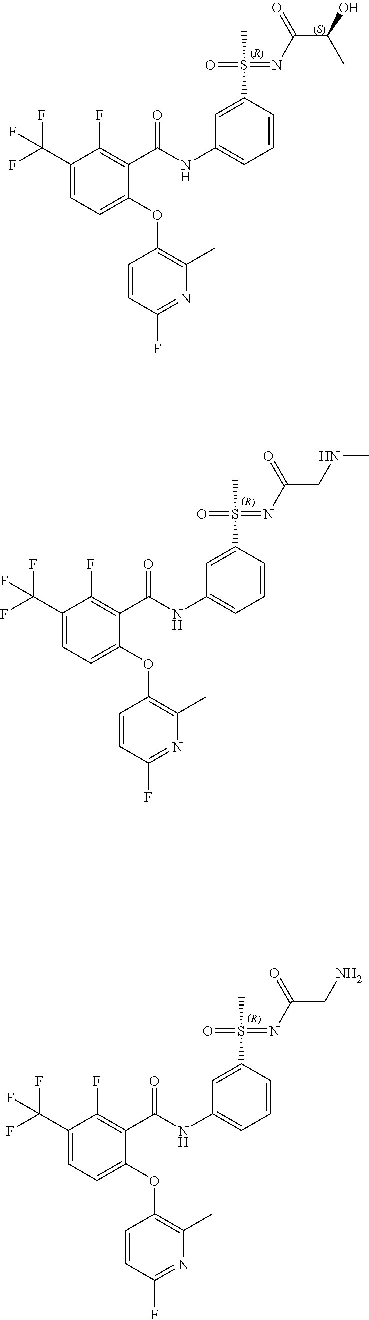

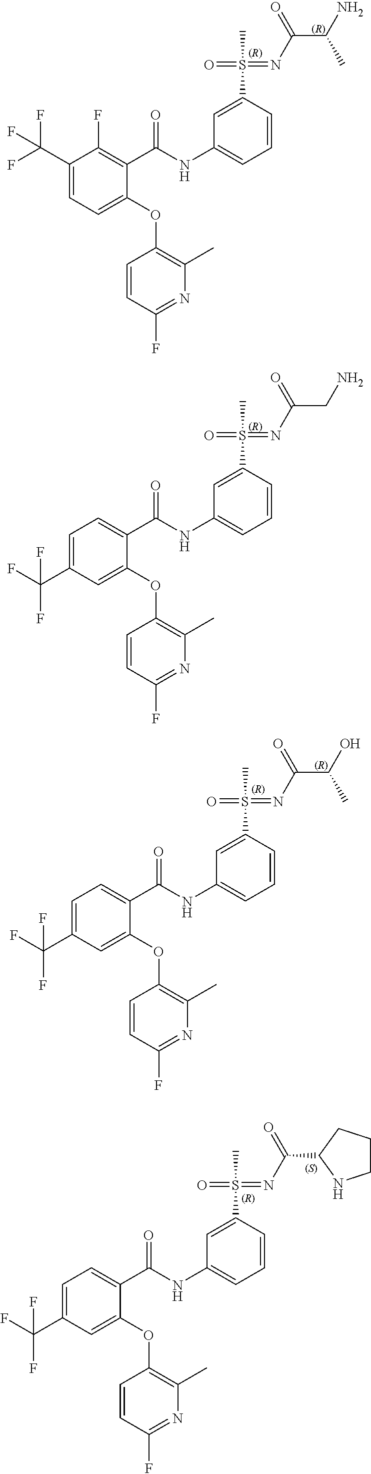

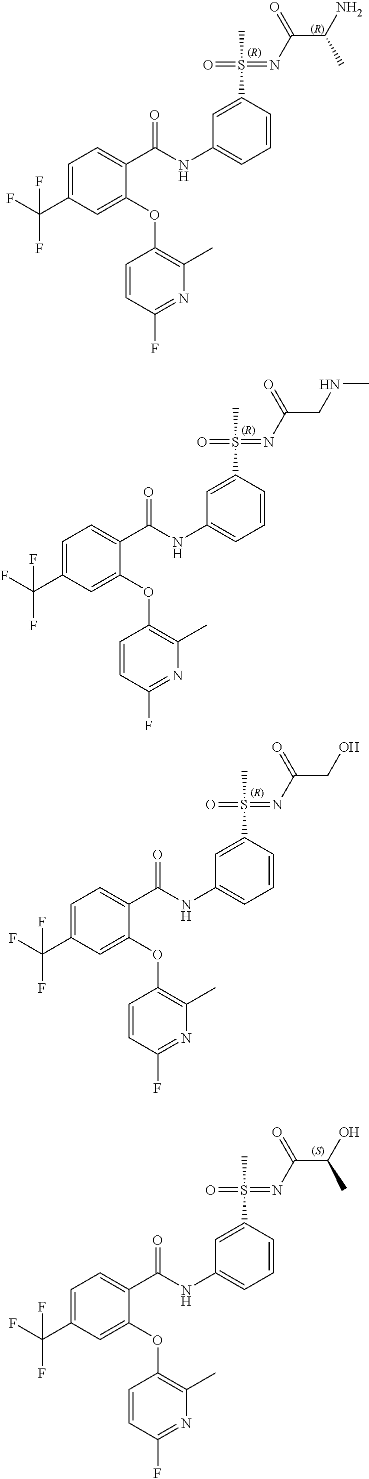

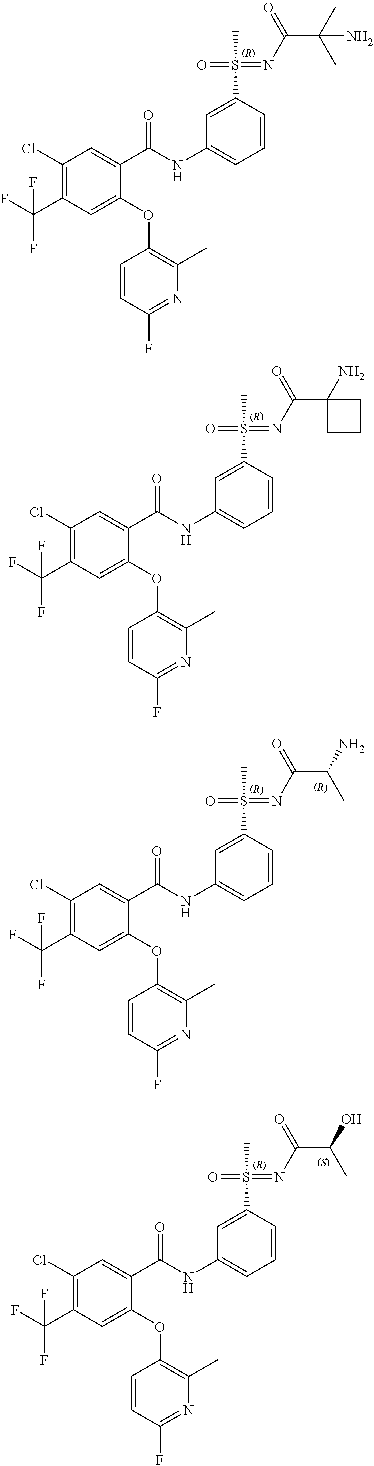

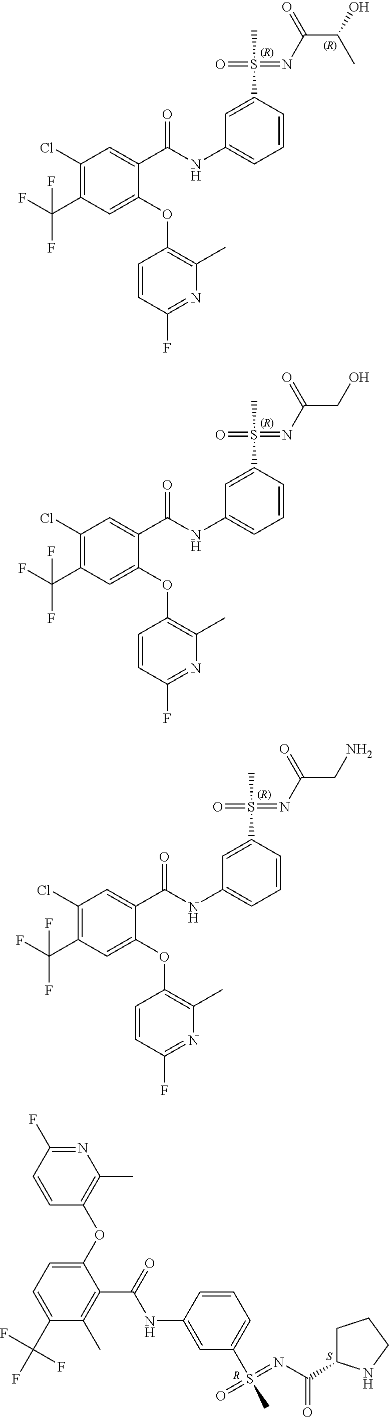

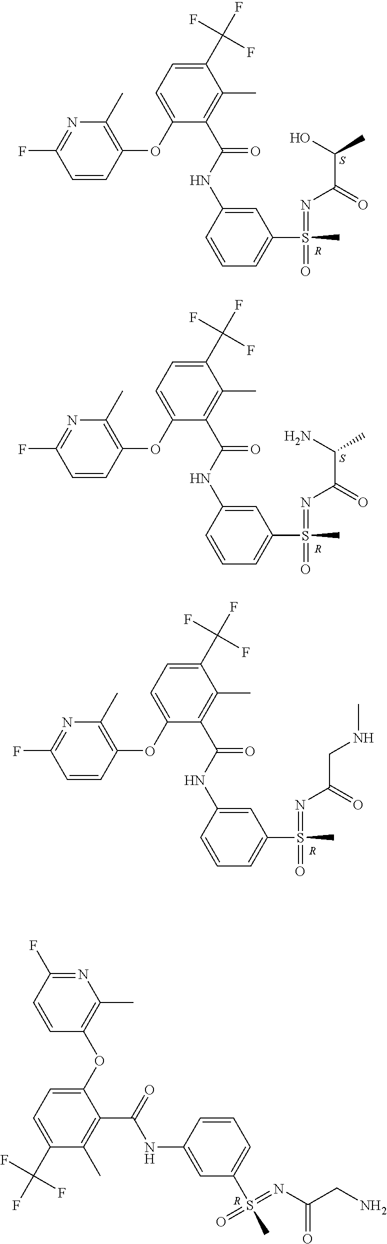

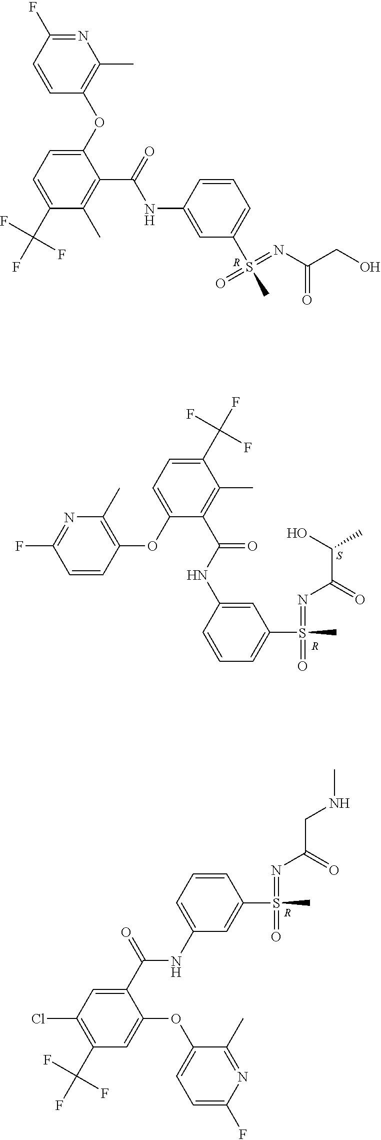

67. A method of treating a condition in a subject, the method comprising providing to a subject having a condition, a compound selected from the compounds recited in claims 51-66.

68. A compound of Formula (I): 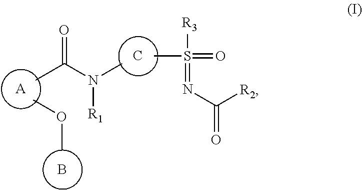
and pharmaceutically acceptable salts, hydrates and solvates thereof, wherein:
A is a substituted or unsubstituted heteroaryl ring comprising at least one heteroatom selected from a group consisting of O, S, or N;
wherein the one or more substitutions on A are selected from H, —OH, halo, C₁-C₈-alkyl, C₁-C₈ fully or partially fluorinated fluoroalkyl, C₂-C₈ branched alkyl, alkenyl, alkynyl, haloalkyl, alkoxy, cycloalkoxy, haloalkoxy, nitro, cyano, —C(R′)(R′)-cycloalkyl, C(R′)(R′)-aryl, —NR′R″, substituted or unsubstituted 3-8 membered cycloalkyl, 3-8 membered cycloalkenyl, or 3-6 membered heterocycloalkyl, wherein the 3-6 membered heterocycloalkyl comprises at least one heteroatom independently selected from O, S, and N;
B is substituted or unsubstituted aryl or heteroaryl, wherein the substitutions are selected from the group consisting of substituted or unsubstituted C₁-C₈ alkyl, deuterated C₁-C₄ alkyl wherein the alkyl chain may be fully or partially deuterated, halo C₁-C₄ alkyl where the alkyl chain is fully or partially halogenated, C₃-C₁₀ cycloalkyl, halogen, cyano, nitro, C₁-C₈ alkoxyl, haloalkoxyl wherein the haloalkyoxyl chain may be fully or partially halogenated, and arylalkoxyl;
C is substituted or unsubstituted aryl or heteroaryl, wherein the one or more substitutions are independently selected from a group consisting of halo, C₁-C₈ alkyl, haloalkyl, alkoxy;
R₁ is selected from a group consisting of: H and C₁-C₄ alkyl;
R₂ is selected from a group consisting of: —(CH₂)ₙRₐ, —(CR_{b}R_{c})ₙRₐ, —(CR_{b}R_{c})ₙ—(OCH₂CH₂)ₙRₐ, —(CR_{b}R_{c})ₙ—(NR′CH₂CH₂)ₙRₐ, —(CR_{b}R_{c})ₙ—(NR′CH₂CH₂)ₙRₐ, and —(CR_{b}R_{c})ₙ—(NHCH₂CH₂NH)ₙRₐ;
wherein Rₐ is H, C₁-C₈-alkyl or branched alkyl, C₃-C₈ cycloalkyl, aryl, heteroaryl, 4-7 membered heterocyclyl, alkenyl, alkynyl, haloalkyl, OH, alkoxy, cycloalkoxy, haloalkoxy, —(OCH₂CH₂)ₙR_{b}, —NR′R″, —COONR′R″, —COOR′R″, alkylsulfonyl, arylsulfonyl, or —SO₂NR′R″;
R_{b} is H, F, C₁-C₆-alkyl, haloalkyl, branched alkyl, aryl, heteroaryl, 3-7 membered carbocyclyl or heterocyclyl with one or more heteroatoms;
R_{c} is H, C₁-C₆ alkyl, or F;
R_{c} and R_{b} together optionally form a 3-6 member carbocyclic or heterocyclic ring;
R₃ is selected from a group consisting of C₁-C₃ alkyl, C₃-C₄ cycloalkyl, haloalkyl, halocycloalkyl, aryl, heteroaryl, and heterocyclyl.

69. The compound of claim 68, wherein the compound is a compound of Formula (II): 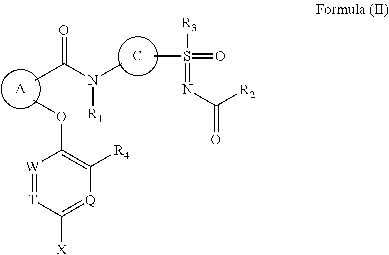
wherein: A, C, R₁, R₂, and R₃ are described above; Q, T and W are independently N or CR₅;
R₅ is H, halogen, —CD₃, alkyl, cycloalkyl, haloalkyl, alkoxy, haloalkoxy, cyano, —CF₃, —OCF₃, or cycloalkoxy;
R₄ is H, —OH, halo, —CD₃, C₁-C₆-alkyl, branched alkyl, haloalkyl where the alkyl chain is fully or partially halogenated, alkoxy, arylalkoxy, cycloalkoxy, haloalkoxy, cyano, —CH₂-cycloalkyl, —CH(CH₃)-cycloalkyl, trifluoromethyl, cyclopropylmethyl, 3-6 membered cycloalkyl or 3-6 membered heterocycloalkyl, any of which may have one or more substituents, wherein the 3-6 membered heterocycloalkyl comprises at least one heteroatom independently selected from O, S, and N;
X is H, halo, —CD₃, alkyl, haloalkyl, alkoxy, cycloalkoxy, haloalkoxy, cyano, —CF₃, or —OCF₃;
A, C, R₁, R₂, and R₃ are described in claim 68.

70. The compound of claim 69, wherein the compound is a compound of Formula (III): 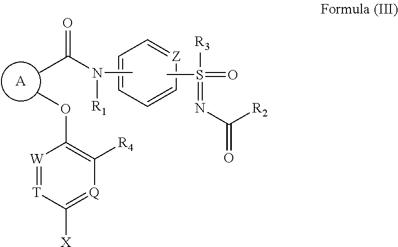
wherein, Z is CH, N, CF, or N0;
A, W, T, Q, X, R₁, R₂, R₃, and R₄, are described in claims 68 and 69.

71. The compound of claim 68, wherein A is substituted or unsubstituted 5-6 membered heteroaryl with one or more heteroatom.

72. The compound of claim 71, wherein the heteroaryl includes a N as the one or more heteroatom.

73. The compound of claim 72, wherein the heteroatom N is in the form of an N-oxide, wherein the N-oxide is selected from a group consisting of: pyridyl N-oxide, pyrazinyl N-oxide and pyrimidinyl N-oxide.

74. The compound of claim 68, wherein A is substituted or unsubstituted pyridyl, pyrimidinyl, pyrazinyl, or pyridazinyl.

75. The compound of claim 68, wherein A is a 6-membered heteroaryl comprising at least one heteroatom selected from N.

76. The compound of claim 68, wherein A is 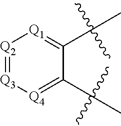
wherein Q₁, Q₂, Q₃ and Q₄ are independently selected from a group consisting of: N, N⁺O⁻, or CR₆;
wherein at least two of Q₁, Q₂, Q₃ and Q₄ are CR₆; R₆ is H, —OH, halo, —CD₃, alkyl, haloalkyl, alkoxy, haloalkoxy, cyano, —CF₃, —OCF₃, substituted or unsubstituted 5 or 6 membered ring heterocyclyl or heteroaryl, saturated heterocyclyl, or partially unsaturated heterocyclyl, O-aryl, O-heteroaryl, O-cycloalkyl, or O-cycloheteroalkyl.

77. The compound of claim 68, wherein A is 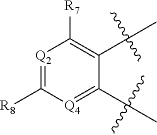
wherein,
Q₂ and Q₄ are independently N or N⁺O⁻;
Q₂ is N or N⁺O⁻ and Q₄ is CR₆; or
Q₂ is CR₆ and Q₄ is N or N⁺O⁻;
R₇ and R₈ are independently selected from the group consisting of H, —OH, halo, —CD₃, substituted or unsubstituted C₁-C₆ alkyl, branched alkyl, alkenyl, alkylnyl, haloalkyl, alkoxy, cycloalkyl, heterocyclyl, NH₂, NHR′, NR′R″, aryl, Heteroaryl, —CF₂CH₃, and —CF₂CF₃;
R₆ is described in claim 76.

78. The compound of claim 69, wherein A is 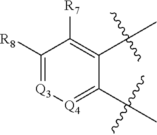
wherein,
Q₃ and Q₄ are N;
Q₃ is N, N⁺O⁻; Q₄ is CR₆; or
Q₃ is CR₆, Q₄ is N or N⁺O⁻; wherein R₇ and R₈ are defined in claim 77.

79. The compound of claim 67, wherein A is 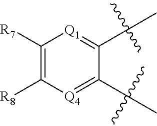
wherein
Q₁ and Q₄ are independently N or N⁺O⁻;
Q₁ is N or N⁺O⁻ and Q₄ is CR₆; or
Q₁ is CR₆ and Q₄ is N or N⁺O⁻; wherein R₆, R₇, and R₈ are defined in claim 77.

80. The compound of claim 68, wherein A is 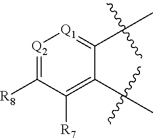
wherein Q₁ and Q₂ is N; wherein R₇ and R₈ are defined in claim 77.

81. The compound of claim 68, wherein A is 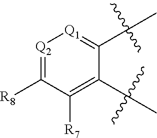
wherein Q₁ is CR₆, and Q₂ is N; R₆ is defined in claim 76, and R₇ and R₈ are defined above in claim 77.

82. The compound of claim 68, wherein A is 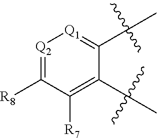
wherein, Q₁ is N; Q₂ is CR₆; R₆ is defined in claim 9, and R₇ and R₈ are defined above in claim 77.

83. The compound of claim 68, wherein R₁ is H.

84. The compound of claim 69, wherein T is N.

85. The compound of claim 69, wherein T is N, W is CH, and Q is CH.

86. The compound of claim 69, wherein R₄ is methyl.

87. The compound of claim 69, wherein X is F.

88. The compound of claim 68, wherein R₃ is methyl.

89. The compound of claim 68, wherein R₂ is H, methyl, —CH₂—NH₂, —CH₂—NH—CH₃, —CH₂—OH, —CH(NH₂)(CH₃), —CH₂—OH, —CH(OH)(CH₃), —CH(CF₃)(NH₂), —CH₂—O—CH₃, amino-cycopropyl, pyrrolidine, azetidine, oxetane, tetrahydrofuran, or hydroxypyrrolidone.

90. The compound of claim 70, wherein Z is N or CH.

91. The compound of claim 68, wherein the compound is selected from a group consisting of:

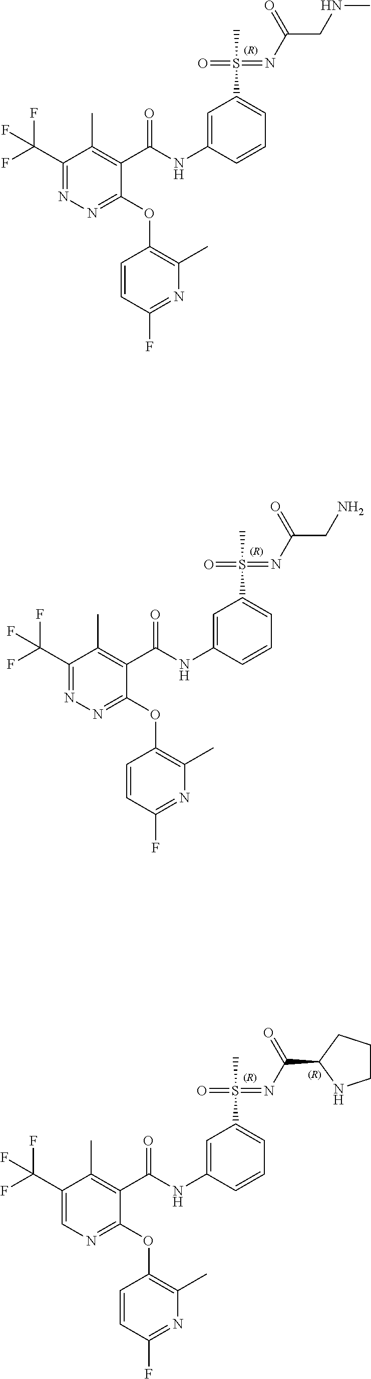

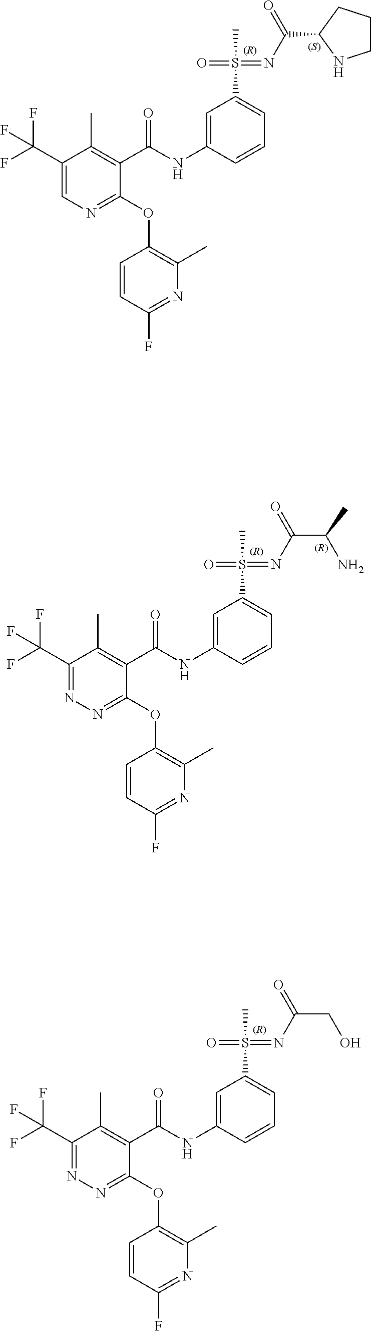

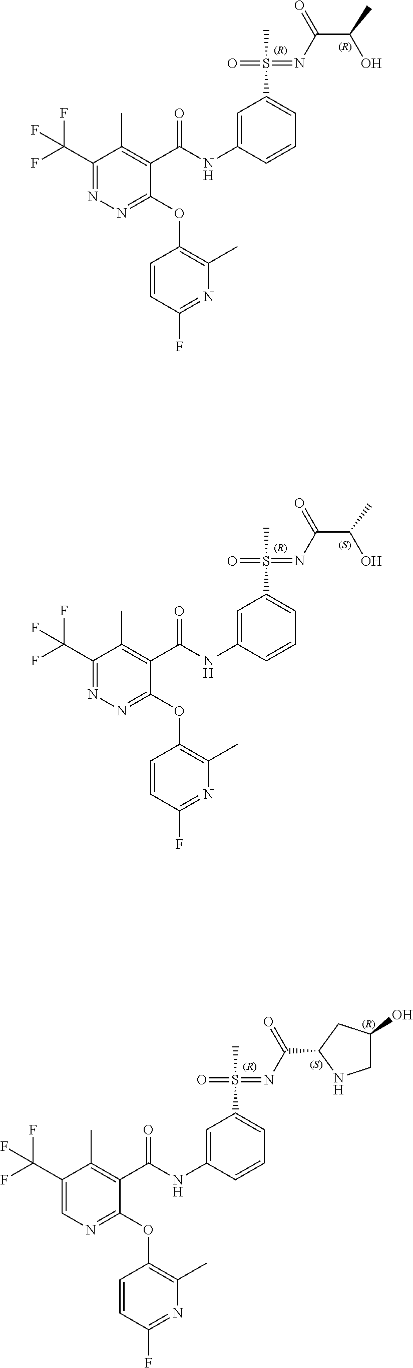

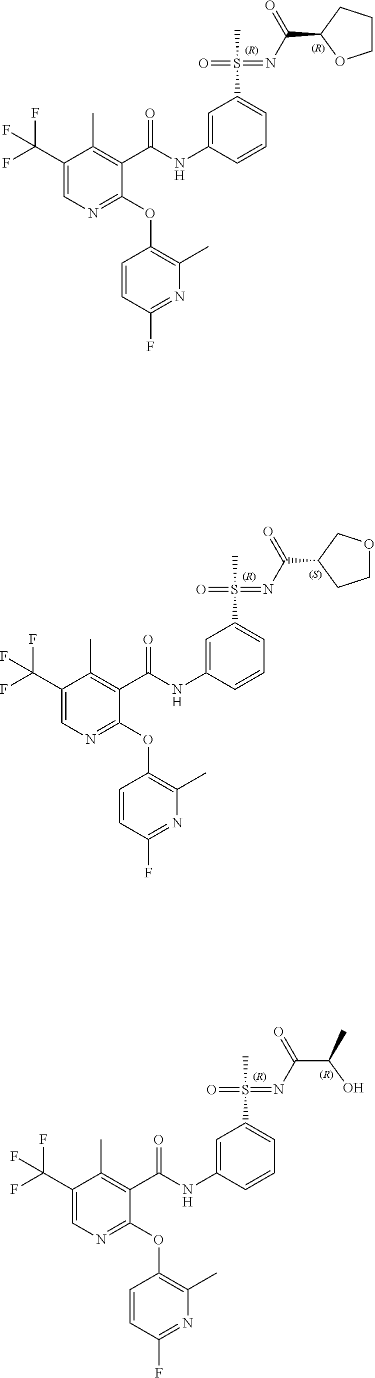

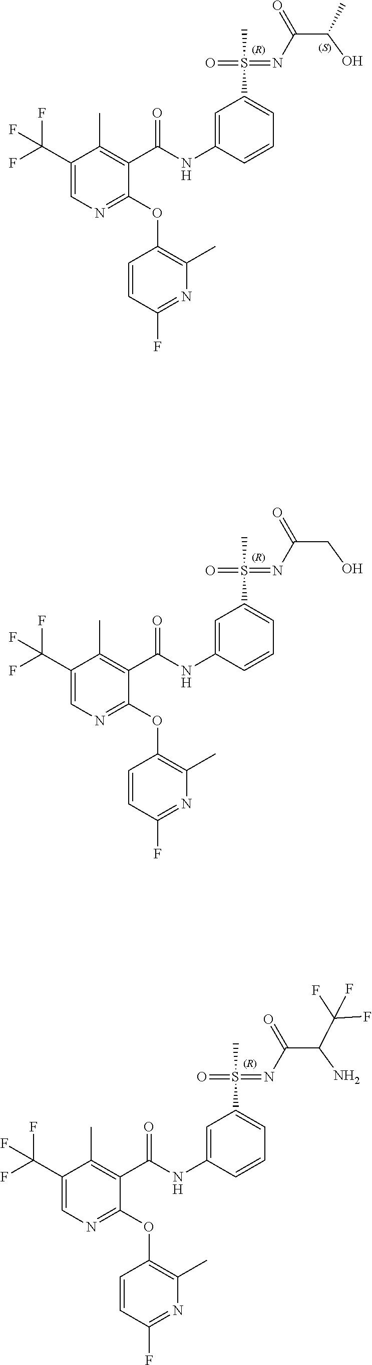

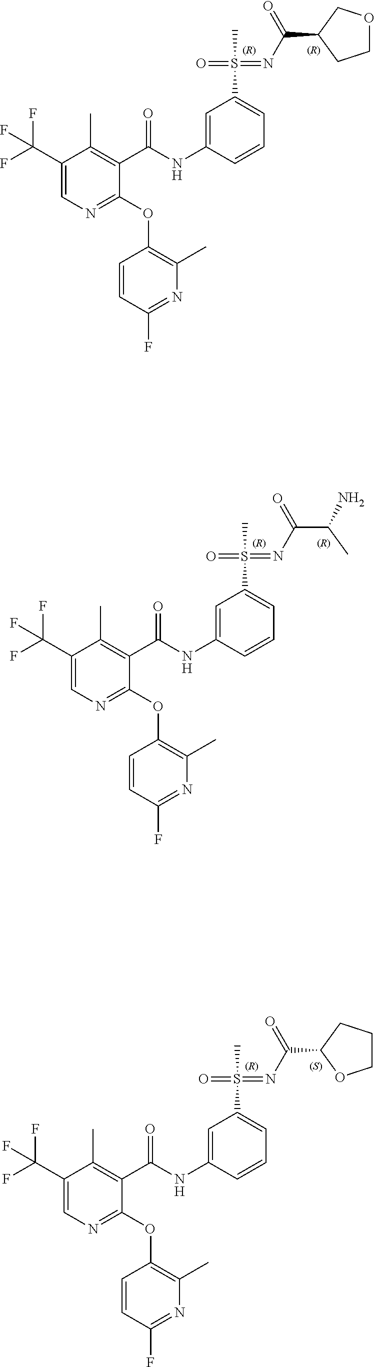

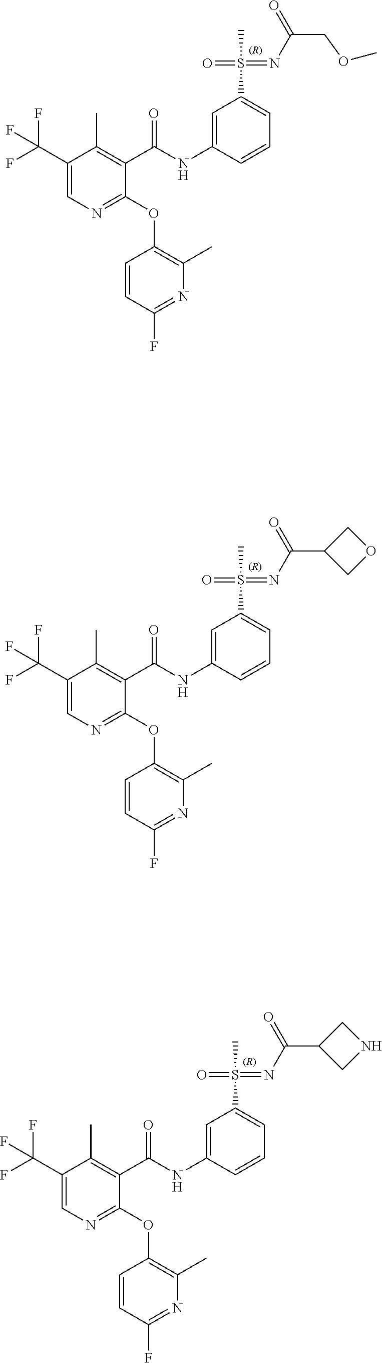

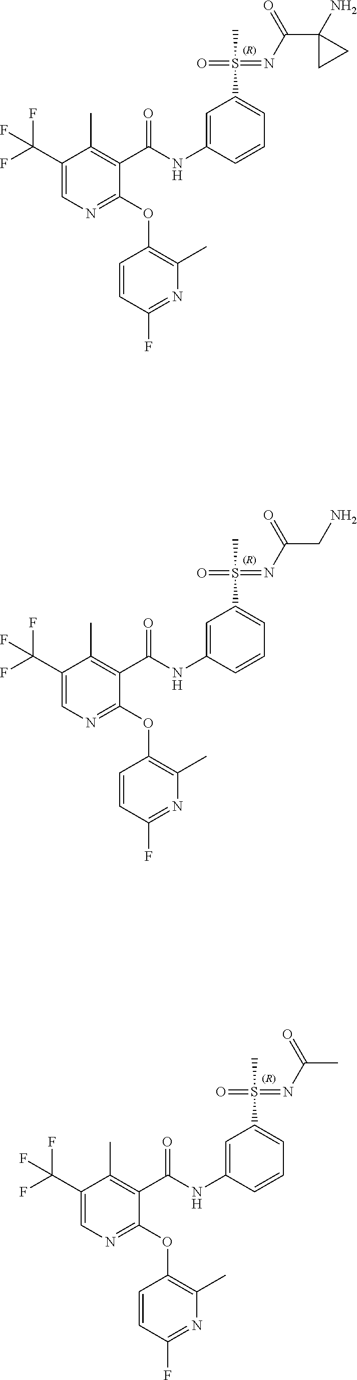

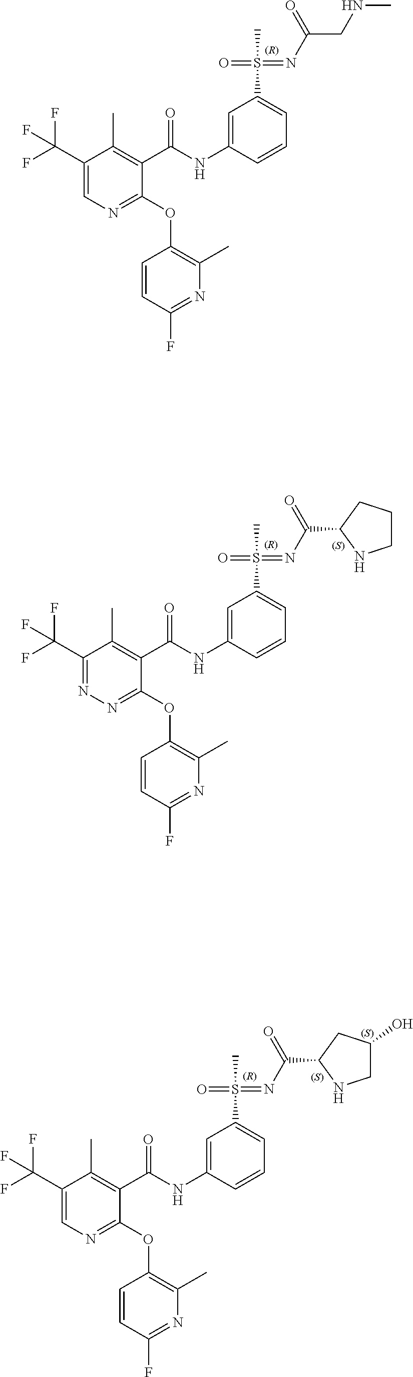

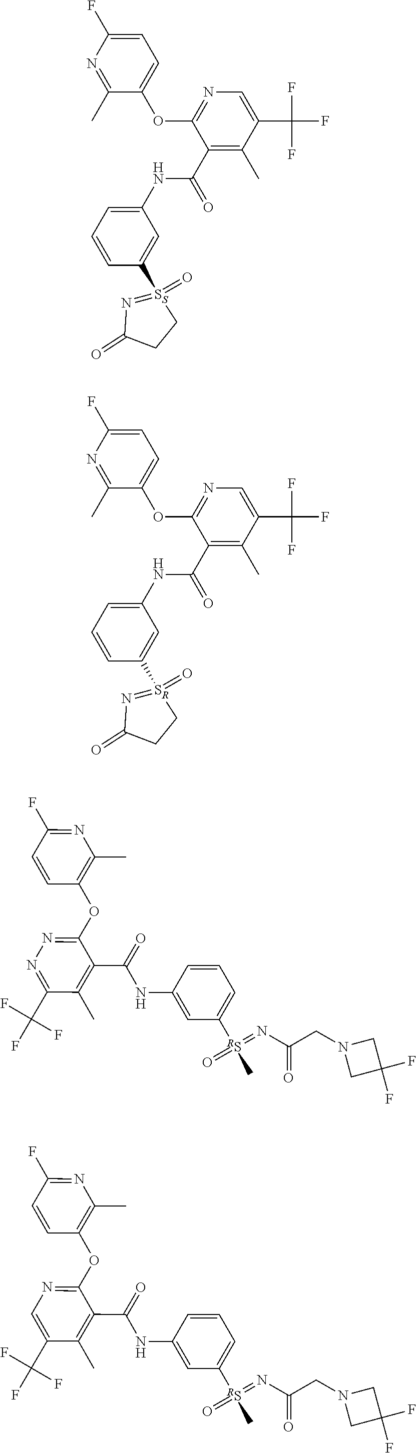

92. A method of treating a condition in a subject, the method comprising providing to a subject having a condition, a compound selected from the compounds recited in claims 68-91.
